SEQUENCE LISTING (213 sequences)

SEQ ID NO: 1 - DNA, 747 bp, synthetic construct
  source 1..747: mol_type = other DNA
ttaaaacagctctggggttgttcccaccccagaggcccacgtggcggccagtactccggtattacggtacccttgtacgcctgttttatactcccttcccctgtaacttagaagcatacaaaccaagttcaatagaagggggtacaaaccagtaccaccacgaacaagcactcctgtttccccggtgacattgcatagactgtacccacggttgaaagcgatcgatccgttacccgctcctgtacttcgagaagcctagtatcatcttggaatcttcgatgcgttgcgctcagcactcaaccccagagtgtagcttaggctgatgagtctggacgtcccccaccggcgacggtggtccaggctgcgttggcggcctacctgtggcccaaagccacaggacgctagttgtgaacaaggtgtgaagagcctattgagctacaagagagtcctccggcccctgaatgcggctaatcctaaccacggagcaggcagttgcaaaccagcaaccggcctgtcgtaacgcgcaagtctgtggcggaaccgactactttgggtgtccgtgtttccttttatttttacaatggctgcttatggtgacaatcatagattgttatcataaagcgacttggattggccatccggtgaaagtaaaacacattgtttacttgtttgttggattcactccaattaacacttttacttacaaactcattacaacaactctattaattagagataagcatcaca

SEQ ID NO: 2 - DNA, 612 bp, synthetic construct
  source 1..612: mol_type = other DNA
tttcccctgttcgtaactaagtgtgtgcccaatctcctcactcctgctggcttcaccgaccggcagtgtccaaaatgctaggtgaatcccctccctttcctctgggcttctgcccagcttcctccccccagcctgacgtgacacaggctgtgcaaagaccccgcgaaagctgccaaaagtggcaattgtgggtcccccctttgtaaaggcgtcgagtctttctccctcaaggctagacccgtcagtgaattctgtcgggcaactagtgacgccactgcacgcctctgacctcggccgcggagtgctgccccccaagtcgtgcccctgaccacaagttgtgctgtctggcaaacattgtctgtgagaatgttccgctgtggctgccaagcctggcaacaggctgccccagtgtgcgtagttctcatccagacttcggtctggcaacttgctgttaagacacggcgtaaggggcgtgtgccaacgccctggaacgagtgtccactctaataccccgaggaatgctacgcaggtacccctggttcgccagggatctgagcgtaggctaattgtctaagggtattttcatttcccattctttctttcttgttcata

SEQ ID NO: 3 - DNA, 439 bp, synthetic construct
  source 1..439: mol_type = other DNA
tccccggcatgagaggaatagactctttcagggttgaagccacgagtgtcgttacccgcactggtactacgcaaagcctagtaacatcttgaaactctttttggttggtcgttccactagttaccccctagtagacctggcagatgaggcaggacgctccccactggcgacagtggtcctgcctgcgtggctgcctgcacacccttcggggtgtgaagccaaaagaaagacaaggtgtgaagagccccgtgtgctaccagtgaatcctccggcccctgaatgcggctaatcttaccccacagctattgcacacaatccagtgtgtatgtagtcgtaatgagcaattgtgggacggaaccgactactttgggtgtccgtgtttccttttattcccatgtttctgcttatggtgacaatactgacgtatagtgttgttacc

SEQ ID NO: 4 - DNA, 627 bp, synthetic construct
  source 1..627: mol_type = other DNA
ttaaaacagcggatgggtatcccaccatccggcccactgggtgtagtactctggtacattgtacctttgtacgcctgttttccccctcttgtacccgcccttcaagctccttgcccaagtaacgttagaagtttgaacattggtacaataggaagcatcacatccagtggtgtactgtacaaacacttctgttgccccggagcgaggtatagatggtccccaccgtcaaaagcctttaaccgttatccgccaatcaactacgtaatggctagtagcaccttggatttaagttggcgttcgatcaggtggtaacccccactagtttggtcgatgaggctaggaattccccacgggtgaccgtgtcctagcctgcgtggcggccaacccagcatccgctgggacgccaatttaatgacatggtgtgaagacctgcatgtgcttgattgtgagtcctccggcccctgaatgcggctaaccctaaccccggagccttgcagcacaatccagtgttgttaaggtcgtaatgagcaattctgggatgggaccgactactttgggtgtccgtgtttcttatttttcttgaatttttcttatggtcacagcatatatacattatatactgtgatc

SEQ ID NO: 5 - DNA, 737 bp, synthetic construct
  source 1..737: mol_type = other DNA
ttaaaatagcctcagggttgttcccaccctgagggcccacgtggtgtagtactctggtattacggtacctttgtacgcctattttatacccccttccccaagtaatttagaagcaagcacaaaccagttcagtagtaagcagtacaatccagtactgtaatgaacaagtacttctgttaccccggaagggtctatcggtaagctgtacccacggctgaagaatgacctaccgttaaccggctacctacttcgagaagcctagtaatgccgttgaagttttattgacgttacgctcagcacactaccccgtgtgtagttttggctgatgagtcacggcactccccacgggcgaccgtggccgtggctgcgttggcggccaaccaaggagtgcaagctccttggacgtcatattacagacatggtgtgaagagcctattgagctaggtggtagtcctccggcccctgaatgcggctaatcctaactccggagcatatcggtgcgaaccagcacttggtgtgttgtaatacgtaagtctggagcggaaccgactactttgggtgtccgtgtttcctgttttaacttttatggctgcttatggtgacaatttaacattgttaccatatagctgttgggttggccatccggattttgttataaaaccatttcctcgtgccttgacctttaacacatttgtgaacttctttaaatcccttttattagtccttaaatactaaga

SEQ ID NO: 6 - DNA, 821 bp, synthetic construct
  source 1..821: mol_type = other DNA
ttcaaacagcctgggggttgtacccacccctggggcccacgtggcgctagtactctggtacgttagtacctttgtacgcctgttttcccctcccttaaacaaattaagattaccactactgaggggagtagtccgactccgctccggtactgccgcaccagtactccggtacacttagtaccctagtacggagtagatggtatccccaccccgcaacttagaagcatgcaaacaaaccgaccaataggcgcacgatatccagtcgtgtttcggtcaagcacttctgtctccccggtccgaaaggatcgttacccgcccgacccactacgagaagcccagtaactggccaagtgattgcgaagttgcgctcagccacaaccccagtggtagctctggaagatggggctcgcgtctcccccgtggtgacacggtcgcttgcccgcgtgtgcttccgggttcggcctacgccgttcacttcaatgtcacgtaaccagccaagagcctattgtgctgggacggttttcctccggggccgtgaatgctgctaatcccaacctccgagcgtgtgcgcacaacccagtgttgctacgtcgtaatgcgtaagttggaggcggaacagactactttcggtaccccgtgtttcctttaaattttattcattattttatggtgacaattgctgagatctgcgaattagcgactctgccgttgaatattgctctgtactatttggttgcattccacaaaacctctgacatccccagtacatacattactttacttgtttacctcaatctaaagcacaagctagataatacaaa

SEQ ID NO: 7 - DNA, 822 bp, synthetic construct
  source 1..822: mol_type = other DNA
tttaaacagcctgggggttgttcccacccctggggcccacgtggcgctagtactctggtacgctagtacctttgtacgcctgtttttcccctcccttaaataaatcaaggttgccactactgaggggagtagtccgactccgctccagcaatgctgcaccagtgcactggtacgctagtaccttttcacggagtagatggtatcccttaccccggaacctagaagattgcacacaaaccgaccaataggcgcaccgcatccagccgtgcagcggtcaagcacttctgtctccccggtctgtaaagatcgttatccgcccgacccactacgaaaagcctagtaactggccaagtgaacgcgaagttgcgctccgccacaaccccagtggtagctctggaagatggggctcgcaccacccccgtggtaacacggttgcctgcccgcgtgtgcttccgggttcggtctcgtgccgttcacttcaacttcacgcaaccagccaagagcctattgtgctgggacggttttcctccggggccgtgaatgctgctaatcccaacctccgagcgtgtgcgcacaatccagtgttgctacgtcgtaacgcgtaagttggaggcggaacagactactttcggtaccccgtgtttcctctcattttatttaatattttatggtgacaattgttgagatttgcgctcttgcaacgttgccattgaatattggcttatactatttggttgccttttacaaaacctctgatatacccagttcttacattgatctgcttgtttttctcaatttgaagtatagactacaaatagcaaa

SEQ ID NO: 8 - DNA, 830 bp, synthetic construct
  source 1..830: mol_type = other DNA
cccccctcccccccttcccttccctttgcaacgcaacaattgtaagtgccctcacctgtcaattgggaccaccactttcagtgaccccatgcgaagtgctgagagaaaggaagctttcttacccttcatttgtgaacccactggtctaagccgcttggaatacgatgagtggaaaagttcattcttaatggagtgaaacatgcttaaatttccagctcgtgctggtctttccagtacggggcggccctgtctggccgtaattcttcagagtgtcacgccacacttgtggatctcacgtgccacatgacagcgctacagctggaactgggtgcttggtgcccatggagtaacagcgaaaagtgttagatcaagccttgcttgggctatgagcctgcggaacaacaactggtaacagttgcctcaggggccgaaagccacggtgttaacagcaccctcatagtttgatccacctcagggtggtgatgtttagcagttagtagttgccaatctgtgttcactgaaatctcggcataccgtgtagtgtacaggggtgaaggatgcccagaaggtacccgtaggtaaccttaagagactatggatctgatctggggccttgtccggagtgctttacacacggctcaaggttaaaaaacgtctagccccacagagcccgagggattcgggttttccctttaaaaacccgactagagcttatggtgacaattattgctgttcagacgaacagtgtaattgttgtctattcacagcagttctatcagagcttttcccacaacggatcttcttggcaagcaaatacagcaggagtcaat

SEQ ID NO: 9 - DNA, 1000 bp, synthetic construct
  source 1..1000: mol_type = other DNA
cactacgttacggttcccgcccgggacaactggtaccccattaggctacaacatggctgaaaagggtattgggtccccccggattgtgtccgttcgtagtgtgtgtaacgtggtttaccatctccactaacattggactaagcatttcatctttcctccccgattgtgtactcacttggctaacgctgggtggtcgcggttgggtccttgatttactttttctcgtctaagcattccgactgtcctccccgattatgtgctcattcagttaactgctgggtggtcatgactaacatcgaggaaccttctgtccacgcttactttgagctccggtcgcttgacgcttgtagggcgatagggttatcttcctgacaacatctttattctacctccataggctctatctatggagacggagtgtggcacccgtcccttctttgggagcttcggtagtgacgccctttgtcactctcgccagccgaggcatgcctggtgccaggtagcaaagaaagcatatgtttaaggacttgactgatttagcgcaagagtttgtagcgatgtccatagtgtctgcggattccccacacggcgacgtgtgccgcggaggccaaaagccacggtgttcacagcacccctatggatgcccacagaccccagtgggcactcttgttgccggactttcaggaaattaggcataggctcttctcaaactcctggcattggactaggtaagaatgccccggaggtaccccagtactccttcgggagtctgggatctgaccgggggccccacaaacatgctttacgtgtttcgtgcggtcaaaaattgtctaactagtcccaaccttgaacaagggattgttctttcctttttattactgagactggcctatggtgacaacagagattgactgtgaatacagttattttctggtgtttatcatttggtttttctccgtgctcttttacctttgtggtatttgttctttagataggcaaa

SEQ ID NO: 10 - DNA, 1000 bp, synthetic construct
  source 1..1000: mol_type = other DNA
cccggccaccccctttcgacgcgggtactgcgatagtgccaccccagtctttcctactcccgactcccgactctaacccaggttccttggaacaggaacaccaatatactcatcccctggatgctgactaatcagaggaacgtcagcattttccggcccaggctaagagaagtagataagttagattccaaattgatttatcatccccttgacgaattcgcgttggaaatgcacctctcacttgccgctcttcacacccattaacttgattcggcctctgtgttgagccccttgttgaagtgcttccctccatcgtgacgtggttggagatctaagtcaaccgactccgacgaaactaccatcatgcctccccgattatgtgatgctttctgccctgctgggtggagcatcctcgggttgagaaaaccttcttcctttttccttggaccccggtcccccggtctaagccgcttggaataagacagggttatcttcacctcttccttcttctacttcatagtgttctatactatgaaagggtatgtgtcgccccttccttctttggagaacacgcgcggcggtctttccgtctctcgaaaagcgcgtgtgcgacatgcagagaaccgtgaagaaagcagtttgcggactagctttagtgcccacaagaaaacagctgtagcgaccacacaaaggcagcggaccccccctcctggcaacaggagcctctgcggccaaaagccacgtggataagatccacctttgtgtgcggcacaaccccagtgccctggtttcttggtgacacttcagtgaaaacgcaaatggcgatctgaagcgcctctgtaggaaagccaagaatgtccaggaggtaccccttccctcgggaagggatctgacctggagacacatcacatgtgctttacacctgtgcttgtgtttaaaaattgtcacagctttcccaaaccaagtggtcttggttttcactctttaaactgatttcact

SEQ ID NO: 11 - DNA, 1000 bp, synthetic construct
  source 1..1000: mol_type = other DNA
cccccggttaccccctttcgacgcgggtactgcgatagtgccaccccagtctttcctactcccgactcccgaccctaacccaggttcctcggaacaggaacacaaatttactcatcccctggatgctgactaatcagaggaacgtcagcattttccggcccaggcttagagaagtagataagttagaatctaaattgatatgacttccccttgacgaattcacgttggaaatgcacccctcacttgccgctcttcacacccactaattgattcggcctactgtgttgagccccttgttgaagtgcttccctccctcgtgacgtggttggagaaatcttgtcacccgactccgacgaaactaccatcatgcctccccgattatgtgatgctttctgccctgctgggtggagtatcctcgggttgagaaatccttcttccttttaccttggaccttggtcccccggtctaagccgcttggaataagacagggttattttcacctcttcttcttctacttcatggtgctctataccatgaaagggtatgtgtcgccccttccttcttggagaactcacgcggcggtcttccgtctctcaaaaagcgcgagtgcgacatgcagagtaacgcgaagaaagcagttcctggcctagctctagtgcccacaagaaaacggctgtagcgaccacacaaaggcagcggaactcccctcctggtaacaggagcctctgcggccaaaagccacgtggattagatccacctttgtgtgcggtgcaaccccagcaccccggtttcttgttgacactctagtgaatccttgaatggcaatctcaagcgcctctgtaggaaagccaagaatgtccaggaggtaccccttcctcgcggaagggatctgacctggagacacatcacacgtgctttacacttgtgcttgtgtttaaaaattgtcacagctttcccaaaccaagtggtcttggttttccttttttatcctactgtcaat

SEQ ID NO: 12 - DNA, 415 bp, synthetic construct
  source 1..415: mol_type = other DNA
tcctcacccatgcttttcctacccccaccacgcccgcatgtttactgctttccttgatgctgcccgtgactacttccatgacctccccaacccaaacctcaaacgccttaagctatcatgggctctctatcaccaatccccttccttcccacccagaacccccccctccctcccctacaacgtgtatgagcaagacgagtttgacaagctcgccgaagccatgctcacccactctcccttccccacatcccattacctcttccaccccctcccgtccaacgcccgcaccatcgtcgagcgtgaggctgactgggatggctgtgatctagaaaagaaatggctcgacctcgtcatagaggacgatgccaagttccttctggagaacggctctctcccgtttggctccacccttgcc

SEQ ID NO: 13 - DNA, 659 bp, synthetic construct
  source 1..659: mol_type = other DNA
ttctcgcctgagtcaacaaagcgagaaacctgcccctccagcgccagacgagcggcataaaacttgaacttctggcatgctccaccacccttttccccattccaacccccattgcgctctcaaggtcgcgctttttcgagactagctcggattcaaaagttcctggcaccctttgccccttcaggcccttaaggtaggaactgaccttgtgctgtgccctcggtgcggaagtgctactgcgtagcgattgtaagatccctttgtggttctgccctggcaaggttatagagtactgtgatccgctgcggatgccatcctggtaacaggacccccagtgtgcgcaacagtatgttcacggtcttccgtgtccaccacattcggaacactgctctcgtgaaacagtgtgtgtccaatccctgcaatcagtatcaactacaccacctaggaatgctaggaaggtaccccggtccgccgggatctgatcctaggctaattgtctacggtggtgctcctttttattttccacttcaattcattggttacaactgctcgatccctgtgtttgctgcccttctctgctctcatcgccattctcaagtgttcacactgtccaagttcctttggttgttcgcttccacttgccactgtcaactcttgtc

SEQ ID NO: 14 - DNA, 711 bp, synthetic construct
  source 1..711: mol_type = other DNA
tcaccctcttttccggtggtccggacccagaccaccgttactccattcagcttcttcggaacctgtttggaggaattaaacgggcacccacccaccttcacccccttttcgtaactaagtgtgtgcccaatctcatgactcctgctgacttcaccgaccagcagtgtccaaaacgctaggtgaatttccttcctccccctctgggcttctgcccagctccctccctccagcctgacgtgccacaggctgtgcaaagaccccgcgaaagctgccaaaagtggcaattgtgggtcccccctttgtaaaggcgtcgagtctttctcccttaaggctagacccgtcagtgaattctgtcgggcaactagtgacgccactgcatgcctccgacctcggccgcggagtgctgccccccaagtcgtgcccctgactacaagttgtgctgtctggcaaacattgtctgtgagaatgttccgctgtggctgccaagcctggtaacaggctgccccagtgtgcgtagttctcatccagacttcggtctggcaacttgctgttaagacacggcgtaaggggcgtgtgccaacgccctggaacgagtgtccactctaataccccgaggaatgctacgcaggtacccctggctccccagggatctgagcgtaggctaattgtctaagggtattttcatttcccactctttctttcttgttcata

SEQ ID NO: 15 - DNA, 650 bp, synthetic construct
  source 1..650: mol_type = other DNA
tctgtcctcaccccatcttcccttctttcctgcaccgttacgcttactcgcatgtgcattgagtggtgcacgtgcttgaacaaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttcatg

SEQ ID NO: 16 - DNA, 625 bp, synthetic construct
  source 1..625: mol_type = other DNA
attctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattaca

SEQ ID NO: 17 - DNA, 549 bp, synthetic construct
  source 1..549: mol_type = other DNA
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctgtcacg

SEQ ID NO: 18 - DNA, 675 bp, synthetic construct
  source 1..675: mol_type = other DNA
tttgctcagcgtaacttctccgggttacgtggagaccaaaaggctacggagactcgggctacggccctggagcacctaggtgctcctaaagacgttagaagttgtacaaactcgcccaatagggccccccaaccaggggggtagcgggcaagcacttctgtttccccggtatgatctcataggctgtacccacggctgaaagagagattatcgttacccgcctcactacttcgagaagcccagtaatggttcatgaagttgatctcgttgacccggtgtttcccccacaccagaaacctgtgatgggggtggtcatcccggtcatggcgacatgacggacctccccgcgccggcacagggcctcttcggaggacgagtgacatggattcaaccgtgaagagcctattgagctagtgttgattcctccgcccccgtgaatgcggctaatcccaactccggagcaggcgggcccaaaccagggtctggcctgtcgtaacgcgaaagtctggagcggaaccgactactttcgggaaggcgtgtttccttttgttccttttatcaagttttatggtgacaactcctggtagacgttttattgcgtttattgagagatttccaacaattgaacagactagaaccacttgttttatcaaaccctcacagaataagataaca

SEQ ID NO: 19 - DNA, 1446 bp, synthetic construct
  source 1..1446: mol_type = other DNA
atggcactgcccgtcaccgcactcctgctcccactggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggaggtggtggcagcggaggtggaggatcaggtggaggcggtagcgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 20 - DNA, 1446 bp, synthetic construct
  source 1..1446: mol_type = other DNA
atggcactcccagtcaccgcacttctgctgcctctcgccctgctgctccatgcagccagacccgacatccagatgacccaaaccaccagctccctgtccgcttccctgggtgaccgggtgactatctcttgccgggcctcccaagacatctccaagtacctgaactggtatcagcaaaagcctgacggcaccgtcaagctcctcatctaccatacctccagactgcactccggggtgcctagcaggttcagcggaagtgggagcggcaccgactacagcctcaccatctccaacctggagcaggaggacatcgccacctacttctgccagcaggggaacacactgccctacaccttcggcggtggcaccaagctggagatcacaggtggcggaggttccggaggaggaggtagtggaggtggaggcagcgaggtgaagctccaggaatccggaccaggtctggtggctcccagccagtccctcagcgtgacctgcaccgtgagcggcgtgtctcttcccgattacggagtgtcctggatcagacagccaccccggaagggtctggagtggctgggagtgatctggggttccgagaccacatactacaactcagccctcaagagccggctcaccatcatcaaggataactccaagtcccaggtcttcctgaagatgaactctctccagaccgacgacaccgccatctactactgcgccaagcactactactacggcgggtcctacgccatggactactggggtcagggaacctccgtcaccgtcagctctatcgaggtgatgtaccctcctccctacctcgacaacgagaagagcaacggcaccatcatccatgtgaaggggaagcatctctgcccctcacccctgttccccggaccatccaagccattctgggtgctggtggttgttggtggggtcctggcttgctactcactcctggtcaccgtcgccttcatcatcttctgggtgcggtcaaagaggtcccggctcttgcactccgattacatgaacatgactccaaggaggcctggtcccacacggaagcactaccaaccatatgccccaccacgcgacttcgctgcttaccggagccgggtcaagttcagtcggagtgcagacgccccagcctaccagcagggccagaaccaactctacaacgagcttaatctgggtcgccgggaggagtatgacgtgctcgataagagaaggggccgggatcctgagatgggcggtaagcccagacggaagaaccctcaggaggggttgtataatgagctccagaaggacaagatggccgaggcatactccgagatcggcatgaaaggtgagcggaggagaggcaaggggcatgacggcctgtaccaggggctcagcacagccaccaaggatacctatgacgcactccacatgcaggcactgcctccacgg

SEQ ID NO: 21 - DNA, 1446 bp, synthetic construct
  source 1..1446: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 22 - DNA, 1446 bp, synthetic construct
  source 1..1446: mol_type = other DNA
atggcccttcccgtcaccgctctcctcctgccactggccttgctgctgcacgctgcacggccagacatccagatgacccagacaaccagctctctgtcagcctctctcggcgatcgcgtcacaatcagctgccgcgcttcccaagacatctccaagtacctgaactggtaccagcaaaagcccgacggcaccgtgaagctgctcatctaccacacctccagactgcatagcggggtgcccagcagattcagtggctcaggctcaggcaccgactacagcctgaccatctccaacctggagcaggaggacattgccacatacttctgccagcagggcaacaccctgccctacaccttcggaggcggcacaaagctggagatcaccggtggaggagggagtggaggaggaggcagtggtggcggaggttccgaggtgaagctccaggaatcaggtccaggactggtcgccccttcccagtccctgtccgtcacctgcaccgtgagtggcgtcagcctcccagactacggtgtgtcttggatccgccaacctcctcgcaaaggcctggaatggctcggcgtcatctggggaagcgagacaacctactataactccgcactgaagtcccgcctcaccatcatcaaggataatagcaagagccaggtcttcctcaagatgaactccctgcagaccgacgataccgccatctactactgtgccaagcactactactacggaggttcttacgccatggattactggggacagggaacctctgtcaccgtcagctccatcgaggtcatgtatccaccaccctacctggacaacgaaaagagcaatggcaccatcatccacgtgaaggggaagcacctctgcccctcacccctgttccctggtccctccaagcctttctgggtcctggtcgtcgtgggaggcgtgttggcctgttactccctgctcgtcaccgtcgccttcatcatcttctgggttaggagtaagcggtcccggcttctgcactctgactacatgaacatgacacccagaagacctgggccaacccggaagcactaccagccctacgctccacccagggactttgcagcctacaggtcccgcgtcaagttctcccggtctgctgacgcacctgcctaccagcagggccaaaaccagctctacaacgagttgaacctcggcagacgggaggagtacgacgtcctcgacaaaaggcggggtcgggatcctgagatgggcggtaagccaaggcggaagaacccacaggaaggcctctataatgagctccagaaggataagatggctgaggcctactccgagatcgggatgaagggcgaaaggagacggggtaaggggcacgacggcctctatcagggtctgagcaccgccaccaaggacacctacgacgccctgcacatgcaggcactgccacctcgg

SEQ ID NO: 23 - DNA, 1446 bp, synthetic construct
  source 1..1446: mol_type = other DNA
atggctctgccagtgaccgcactgctgctgcccttagccttactccttcacgcagccaggcccgacatccagatgacccagaccaccagctccctttccgcaagcctcggcgacagggtcaccatctcctgtcgggccagccaggacatcagcaagtacctgaactggtaccagcagaagcccgacggcaccgtgaagctgctgatctaccacacctcacggctgcactcaggcgtgccctcacggtttagcggatcaggcagcggcaccgactacagcctgactatcagcaacctggagcaggaggacatcgccacctacttctgccagcagggcaacaccctgccctacaccttcggaggcggcaccaagctggagatcaccggtggcggtggttcaggtggcggaggctcaggaggaggcggcagcgaggtgaagctgcaggagtcaggtccaggactggtggcacccagccagagcctgagcgtgacttgcaccgtgtcaggcgtgagcctgccagactacggcgtgagctggatccggcagcctcctcggaagggcttagagtggctgggcgtgatctggggcagcgagaccacctactacaactcagccctgaagagccggctgaccatcatcaaggacaacagcaagagccaggtgttcctgaagatgaacagcctgcagaccgacgacaccgccatctactactgcgccaagcactactactacggcggcagctacgccatggactactggggacagggtaccagcgtgaccgtgagcagcatcgaggtgatgtaccctcctccctacctggacaacgagaagagcaacggcaccatcatccacgtgaagggcaagcacctgtgccctagccctttattccccggcccctcaaaacccttctgggtgctggtcgtcgtcggtggcgtgctggcatgctacagcctgctggtgaccgtggccttcatcatattctgggtccggtcaaagcggagccggttactgcacagcgactacatgaacatgactccacggcgtccaggtcccactcggaagcactaccaaccctacgctcctccccgtgactttgctgcctaccgtagccgggtgaagttctccaggagcgccgatgccccagcctaccagcagggccagaaccagctctacaatgagcttaaccttggcaggcgggaggagtacgacgtgctggacaagaggaggggccgtgatcccgagatgggaggcaagccccgtaggaagaatccccaggagggcctttacaacgagctccagaaggacaagatggccgaggcctacagcgagatcggcatgaagggagagcgtaggcgtggaaagggccacgacggcctgtaccagggcctgagcactgctaccaaggacacctacgacgccctgcacatgcaggctcttccaccccgg

SEQ ID NO: 24 - DNA, 1467 bp, synthetic construct
  source 1..1467: mol_type = other DNA
atgctcctcctggtgaccagcttgctcctgtgcgaactgccacaccccgccttcctcctcatccccgatatccagatgacccagaccacctcctccctgagcgcaagcctcggcgatcgggtgaccatctcatgcagggcctcccaggacatctccaagtatctgaactggtatcagcagaagcctgacggcaccgtcaagctgctcatctaccacacctcacggctgcactcaggcgtcccctcaagattcagcggtagcggatccgggaccgactactcccttaccatcagcaacctggagcaggaggatatcgccacatacttctgccagcagggtaacaccctgccctataccttcggcggtgggaccaagctggagatcaccggttctacatccggatccggcaagcctggtagtggcgagggctccaccaaaggggaggtgaagctgcaggagtccggtccaggtctggtggctccaagtcagtccctgtctgtgacttgcaccgtgtcaggcgtgagcctgcctgactacggggtgagctggatccggcagccacctcggaaggggttggagtggctgggagtcatctggggatccgagaccacctactacaattccgccctcaaaagccgcctcaccatcatcaaggacaactccaagtcccaggtcttcctgaagatgaattccctgcagaccgacgacaccgctatctattactgcgccaagcattactactacggcgggtcctacgccatggactactggggtcaaggcacctccgtcactgtttcctccgcagcagccatcgaggtcatgtatcctcctccctacctcgacaacgagaagtccaacgggaccatcatccacgtgaagggcaagcacctctgcccaagcccactgttcccagggccctccaaaccattctgggtgctcgtggtggtgggtggcgtgctcgcttgctactccctcctggtcaccgtcgccttcatcatcttttgggtccggagtaagcgcagccgcctgctccatagcgactacatgaacatgaccccacggagacctggtcccacccggaaacactaccagccctacgcaccacccagggacttcgctgcctatcggtcccgggttaaattctctaggtccgctgatgccccagcctaccagcagggccagaaccagctgtacaatgagctgaacctgggtagacgggaggagtatgacgtcctggataagcgcagagggagagaccccgagatgggtggaaagcccaggcggaagaatccccaggagggtctctataacgagctccagaaggacaagatggccgaggcctacagcgagatcgggatgaaaggggaaagaaggcggggaaagggccatgacggactgtaccagggtctgtccaccgctaccaaggacacctacgatgcactgcacatgcaggcactgcctcctcgg

SEQ ID NO: 25 - DNA, 1467 bp, synthetic construct
  source 1..1467: mol_type = other DNA
atgctgcttctcgttacatctctgttgctctgcgagctgcctcatccagccttcctcctgattcccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggcagcacctcaggctctgggaagcctggcagcggtgaaggcagcaccaagggtgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccgctgccgctatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 26 - DNA, 1464 bp, synthetic construct
  source 1..1464: mol_type = other DNA
atggcacttccagttacagcacttctgcttccattggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggcagcacctcaggctctgggaagcctggcagcggtgaaggcagcaccaagggtgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccgctgccgctatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 27 - DNA, 1464 bp, synthetic construct
  source 1..1464: mol_type = other DNA
atggcactgcccgtcaccgcactcctgctcccactggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggcagcacctcaggctctgggaagcctggcagcggtgaaggcagcaccaagggtgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccgctgccgctatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 28 - DNA, 1446 bp, synthetic construct
  source 1..1446: mol_type = other DNA
atggcacttccagttacagcacttctgcttccattggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggaggtggtggcagcggaggtggaggatcaggtggaggcggtagcgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 29 - protein, 482 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 489 aa, synthetic construct; identical to SEQ ID NO: 30

SEQ ID NO: 32 - protein, 488 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 488 aa, synthetic construct; identical to SEQ ID NO: 32

SEQ ID NO: 34 - protein, 482 aa, synthetic construct; identical to SEQ ID NO: 29

SEQ ID NO: 35-49 - skipped (15 entries)

SEQ ID NO: 50 - DNA, 2193 bp, synthetic construct
  source 1..2193: mol_type = other DNA
ttaaaacagctctggggttgttcccaccccagaggcccacgtggcggccagtactccggtattacggtacccttgtacgcctgttttatactcccttcccctgtaacttagaagcatacaaaccaagttcaatagaagggggtacaaaccagtaccaccacgaacaagcactcctgtttccccggtgacattgcatagactgtacccacggttgaaagcgatcgatccgttacccgctcctgtacttcgagaagcctagtatcatcttggaatcttcgatgcgttgcgctcagcactcaaccccagagtgtagcttaggctgatgagtctggacgtcccccaccggcgacggtggtccaggctgcgttggcggcctacctgtggcccaaagccacaggacgctagttgtgaacaaggtgtgaagagcctattgagctacaagagagtcctccggcccctgaatgcggctaatcctaaccacggagcaggcagttgcaaaccagcaaccggcctgtcgtaacgcgcaagtctgtggcggaaccgactactttgggtgtccgtgtttccttttatttttacaatggctgcttatggtgacaatcatagattgttatcataaagcgacttggattggccatccggtgaaagtaaaacacattgtttacttgtttgttggattcactccaattaacacttttacttacaaactcattacaacaactctattaattagagataagcatcacaatggcactgcccgtcaccgcactcctgctcccactggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggaggtggtggcagcggaggtggaggatcaggtggaggcggtagcgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 51 - DNA, 2058 bp, synthetic construct
  source 1..2058: mol_type = other DNA
tttcccctgttcgtaactaagtgtgtgcccaatctcctcactcctgctggcttcaccgaccggcagtgtccaaaatgctaggtgaatcccctccctttcctctgggcttctgcccagcttcctccccccagcctgacgtgacacaggctgtgcaaagaccccgcgaaagctgccaaaagtggcaattgtgggtcccccctttgtaaaggcgtcgagtctttctccctcaaggctagacccgtcagtgaattctgtcgggcaactagtgacgccactgcacgcctctgacctcggccgcggagtgctgccccccaagtcgtgcccctgaccacaagttgtgctgtctggcaaacattgtctgtgagaatgttccgctgtggctgccaagcctggcaacaggctgccccagtgtgcgtagttctcatccagacttcggtctggcaacttgctgttaagacacggcgtaaggggcgtgtgccaacgccctggaacgagtgtccactctaataccccgaggaatgctacgcaggtacccctggttcgccagggatctgagcgtaggctaattgtctaagggtattttcatttcccattctttctttcttgttcataatggcactgcccgtcaccgcactcctgctcccactggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggaggtggtggcagcggaggtggaggatcaggtggaggcggtagcgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 52 - DNA, 2073 bp, synthetic construct
  source 1..2073: mol_type = other DNA
ttaaaacagcggatgggtatcccaccatccggcccactgggtgtagtactctggtacattgtacctttgtacgcctgttttccccctcttgtacccgcccttcaagctccttgcccaagtaacgttagaagtttgaacattggtacaataggaagcatcacatccagtggtgtactgtacaaacacttctgttgccccggagcgaggtatagatggtccccaccgtcaaaagcctttaaccgttatccgccaatcaactacgtaatggctagtagcaccttggatttaagttggcgttcgatcaggtggtaacccccactagtttggtcgatgaggctaggaattccccacgggtgaccgtgtcctagcctgcgtggcggccaacccagcatccgctgggacgccaatttaatgacatggtgtgaagacctgcatgtgcttgattgtgagtcctccggcccctgaatgcggctaaccctaaccccggagccttgcagcacaatccagtgttgttaaggtcgtaatgagcaattctgggatgggaccgactactttgggtgtccgtgtttcttatttttcttgaatttttcttatggtcacagcatatatacattatatactgtgatcatggcactgcccgtcaccgcactcctgctcccactggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggaggtggtggcagcggaggtggaggatcaggtggaggcggtagcgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 53 - DNA, 2267 bp, synthetic construct
  source 1..2267: mol_type = other DNA
ttcaaacagcctgggggttgtacccacccctggggcccacgtggcgctagtactctggtacgttagtacctttgtacgcctgttttcccctcccttaaacaaattaagattaccactactgaggggagtagtccgactccgctccggtactgccgcaccagtactccggtacacttagtaccctagtacggagtagatggtatccccaccccgcaacttagaagcatgcaaacaaaccgaccaataggcgcacgatatccagtcgtgtttcggtcaagcacttctgtctccccggtccgaaaggatcgttacccgcccgacccactacgagaagcccagtaactggccaagtgattgcgaagttgcgctcagccacaaccccagtggtagctctggaagatggggctcgcgtctcccccgtggtgacacggtcgcttgcccgcgtgtgcttccgggttcggcctacgccgttcacttcaatgtcacgtaaccagccaagagcctattgtgctgggacggttttcctccggggccgtgaatgctgctaatcccaacctccgagcgtgtgcgcacaacccagtgttgctacgtcgtaatgcgtaagttggaggcggaacagactactttcggtaccccgtgtttcctttaaattttattcattattttatggtgacaattgctgagatctgcgaattagcgactctgccgttgaatattgctctgtactatttggttgcattccacaaaacctctgacatccccagtacatacattactttacttgtttacctcaatctaaagcacaagctagataatacaaaatggcactgcccgtcaccgcactcctgctcccactggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggaggtggtggcagcggaggtggaggatcaggtggaggcggtagcgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 54 - DNA, 2276 bp, synthetic construct
  source 1..2276: mol_type = other DNA
cccccctcccccccttcccttccctttgcaacgcaacaattgtaagtgccctcacctgtcaattgggaccaccactttcagtgaccccatgcgaagtgctgagagaaaggaagctttcttacccttcatttgtgaacccactggtctaagccgcttggaatacgatgagtggaaaagttcattcttaatggagtgaaacatgcttaaatttccagctcgtgctggtctttccagtacggggcggccctgtctggccgtaattcttcagagtgtcacgccacacttgtggatctcacgtgccacatgacagcgctacagctggaactgggtgcttggtgcccatggagtaacagcgaaaagtgttagatcaagccttgcttgggctatgagcctgcggaacaacaactggtaacagttgcctcaggggccgaaagccacggtgttaacagcaccctcatagtttgatccacctcagggtggtgatgtttagcagttagtagttgccaatctgtgttcactgaaatctcggcataccgtgtagtgtacaggggtgaaggatgcccagaaggtacccgtaggtaaccttaagagactatggatctgatctggggccttgtccggagtgctttacacacggctcaaggttaaaaaacgtctagccccacagagcccgagggattcgggttttccctttaaaaacccgactagagcttatggtgacaattattgctgttcagacgaacagtgtaattgttgtctattcacagcagttctatcagagcttttcccacaacggatcttcttggcaagcaaatacagcaggagtcaatatggcactcccagtcaccgcacttctgctgcctctcgccctgctgctccatgcagccagacccgacatccagatgacccaaaccaccagctccctgtccgcttccctgggtgaccgggtgactatctcttgccgggcctcccaagacatctccaagtacctgaactggtatcagcaaaagcctgacggcaccgtcaagctcctcatctaccatacctccagactgcactccggggtgcctagcaggttcagcggaagtgggagcggcaccgactacagcctcaccatctccaacctggagcaggaggacatcgccacctacttctgccagcaggggaacacactgccctacaccttcggcggtggcaccaagctggagatcacaggtggcggaggttccggaggaggaggtagtggaggtggaggcagcgaggtgaagctccaggaatccggaccaggtctggtggctcccagccagtccctcagcgtgacctgcaccgtgagcggcgtgtctcttcccgattacggagtgtcctggatcagacagccaccccggaagggtctggagtggctgggagtgatctggggttccgagaccacatactacaactcagccctcaagagccggctcaccatcatcaaggataactccaagtcccaggtcttcctgaagatgaactctctccagaccgacgacaccgccatctactactgcgccaagcactactactacggcgggtcctacgccatggactactggggtcagggaacctccgtcaccgtcagctctatcgaggtgatgtaccctcctccctacctcgacaacgagaagagcaacggcaccatcatccatgtgaaggggaagcatctctgcccctcacccctgttccccggaccatccaagccattctgggtgctggtggttgttggtggggtcctggcttgctactcactcctggtcaccgtcgccttcatcatcttctgggtgcggtcaaagaggtcccggctcttgcactccgattacatgaacatgactccaaggaggcctggtcccacacggaagcactaccaaccatatgccccaccacgcgacttcgctgcttaccggagccgggtcaagttcagtcggagtgcagacgccccagcctaccagcagggccagaaccaactctacaacgagcttaatctgggtcgccgggaggagtatgacgtgctcgataagagaaggggccgggatcctgagatgggcggtaagcccagacggaagaaccctcaggaggggttgtataatgagctccagaaggacaagatggccgaggcatactccgagatcggcatgaaaggtgagcggaggagaggcaaggggcatgacggcctgtaccaggggctcagcacagccaccaaggatacctatgacgcactccacatgcaggcactgcctccacgg

SEQ ID NO: 55 - DNA, 2276 bp, synthetic construct
  source 1..2276: mol_type = other DNA
cccccctcccccccttcccttccctttgcaacgcaacaattgtaagtgccctcacctgtcaattgggaccaccactttcagtgaccccatgcgaagtgctgagagaaaggaagctttcttacccttcatttgtgaacccactggtctaagccgcttggaatacgatgagtggaaaagttcattcttaatggagtgaaacatgcttaaatttccagctcgtgctggtctttccagtacggggcggccctgtctggccgtaattcttcagagtgtcacgccacacttgtggatctcacgtgccacatgacagcgctacagctggaactgggtgcttggtgcccatggagtaacagcgaaaagtgttagatcaagccttgcttgggctatgagcctgcggaacaacaactggtaacagttgcctcaggggccgaaagccacggtgttaacagcaccctcatagtttgatccacctcagggtggtgatgtttagcagttagtagttgccaatctgtgttcactgaaatctcggcataccgtgtagtgtacaggggtgaaggatgcccagaaggtacccgtaggtaaccttaagagactatggatctgatctggggccttgtccggagtgctttacacacggctcaaggttaaaaaacgtctagccccacagagcccgagggattcgggttttccctttaaaaacccgactagagcttatggtgacaattattgctgttcagacgaacagtgtaattgttgtctattcacagcagttctatcagagcttttcccacaacggatcttcttggcaagcaaatacagcaggagtcaatatggcactgcccgtcaccgcactcctgctcccactggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggaggtggtggcagcggaggtggaggatcaggtggaggcggtagcgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 56 - DNA, 2446 bp, synthetic construct
  source 1..2446: mol_type = other DNA
cactacgttacggttcccgcccgggacaactggtaccccattaggctacaacatggctgaaaagggtattgggtccccccggattgtgtccgttcgtagtgtgtgtaacgtggtttaccatctccactaacattggactaagcatttcatctttcctccccgattgtgtactcacttggctaacgctgggtggtcgcggttgggtccttgatttactttttctcgtctaagcattccgactgtcctccccgattatgtgctcattcagttaactgctgggtggtcatgactaacatcgaggaaccttctgtccacgcttactttgagctccggtcgcttgacgcttgtagggcgatagggttatcttcctgacaacatctttattctacctccataggctctatctatggagacggagtgtggcacccgtcccttctttgggagcttcggtagtgacgccctttgtcactctcgccagccgaggcatgcctggtgccaggtagcaaagaaagcatatgtttaaggacttgactgatttagcgcaagagtttgtagcgatgtccatagtgtctgcggattccccacacggcgacgtgtgccgcggaggccaaaagccacggtgttcacagcacccctatggatgcccacagaccccagtgggcactcttgttgccggactttcaggaaattaggcataggctcttctcaaactcctggcattggactaggtaagaatgccccggaggtaccccagtactccttcgggagtctgggatctgaccgggggccccacaaacatgctttacgtgtttcgtgcggtcaaaaattgtctaactagtcccaaccttgaacaagggattgttctttcctttttattactgagactggcctatggtgacaacagagattgactgtgaatacagttattttctggtgtttatcatttggtttttctccgtgctcttttacctttgtggtatttgttctttagataggcaaaatggcactgcccgtcaccgcactcctgctcccactggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggaggtggtggcagcggaggtggaggatcaggtggaggcggtagcgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 57 - DNA, 2157 bp, synthetic construct
  source 1..2157: mol_type = other DNA
tcaccctcttttccggtggtccggacccagaccaccgttactccattcagcttcttcggaacctgtttggaggaattaaacgggcacccacccaccttcacccccttttcgtaactaagtgtgtgcccaatctcatgactcctgctgacttcaccgaccagcagtgtccaaaacgctaggtgaatttccttcctccccctctgggcttctgcccagctccctccctccagcctgacgtgccacaggctgtgcaaagaccccgcgaaagctgccaaaagtggcaattgtgggtcccccctttgtaaaggcgtcgagtctttctcccttaaggctagacccgtcagtgaattctgtcgggcaactagtgacgccactgcatgcctccgacctcggccgcggagtgctgccccccaagtcgtgcccctgactacaagttgtgctgtctggcaaacattgtctgtgagaatgttccgctgtggctgccaagcctggtaacaggctgccccagtgtgcgtagttctcatccagacttcggtctggcaacttgctgttaagacacggcgtaaggggcgtgtgccaacgccctggaacgagtgtccactctaataccccgaggaatgctacgcaggtacccctggctccccagggatctgagcgtaggctaattgtctaagggtattttcatttcccactctttctttcttgttcataatggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 58 - DNA, 2157 bp, synthetic construct
  source 1..2157: mol_type = other DNA
tcaccctcttttccggtggtccggacccagaccaccgttactccattcagcttcttcggaacctgtttggaggaattaaacgggcacccacccaccttcacccccttttcgtaactaagtgtgtgcccaatctcatgactcctgctgacttcaccgaccagcagtgtccaaaacgctaggtgaatttccttcctccccctctgggcttctgcccagctccctccctccagcctgacgtgccacaggctgtgcaaagaccccgcgaaagctgccaaaagtggcaattgtgggtcccccctttgtaaaggcgtcgagtctttctcccttaaggctagacccgtcagtgaattctgtcgggcaactagtgacgccactgcatgcctccgacctcggccgcggagtgctgccccccaagtcgtgcccctgactacaagttgtgctgtctggcaaacattgtctgtgagaatgttccgctgtggctgccaagcctggtaacaggctgccccagtgtgcgtagttctcatccagacttcggtctggcaacttgctgttaagacacggcgtaaggggcgtgtgccaacgccctggaacgagtgtccactctaataccccgaggaatgctacgcaggtacccctggctccccagggatctgagcgtaggctaattgtctaagggtattttcatttcccactctttctttcttgttcataatggcccttcccgtcaccgctctcctcctgccactggccttgctgctgcacgctgcacggccagacatccagatgacccagacaaccagctctctgtcagcctctctcggcgatcgcgtcacaatcagctgccgcgcttcccaagacatctccaagtacctgaactggtaccagcaaaagcccgacggcaccgtgaagctgctcatctaccacacctccagactgcatagcggggtgcccagcagattcagtggctcaggctcaggcaccgactacagcctgaccatctccaacctggagcaggaggacattgccacatacttctgccagcagggcaacaccctgccctacaccttcggaggcggcacaaagctggagatcaccggtggaggagggagtggaggaggaggcagtggtggcggaggttccgaggtgaagctccaggaatcaggtccaggactggtcgccccttcccagtccctgtccgtcacctgcaccgtgagtggcgtcagcctcccagactacggtgtgtcttggatccgccaacctcctcgcaaaggcctggaatggctcggcgtcatctggggaagcgagacaacctactataactccgcactgaagtcccgcctcaccatcatcaaggataatagcaagagccaggtcttcctcaagatgaactccctgcagaccgacgataccgccatctactactgtgccaagcactactactacggaggttcttacgccatggattactggggacagggaacctctgtcaccgtcagctccatcgaggtcatgtatccaccaccctacctggacaacgaaaagagcaatggcaccatcatccacgtgaaggggaagcacctctgcccctcacccctgttccctggtccctccaagcctttctgggtcctggtcgtcgtgggaggcgtgttggcctgttactccctgctcgtcaccgtcgccttcatcatcttctgggttaggagtaagcggtcccggcttctgcactctgactacatgaacatgacacccagaagacctgggccaacccggaagcactaccagccctacgctccacccagggactttgcagcctacaggtcccgcgtcaagttctcccggtctgctgacgcacctgcctaccagcagggccaaaaccagctctacaacgagttgaacctcggcagacgggaggagtacgacgtcctcgacaaaaggcggggtcgggatcctgagatgggcggtaagccaaggcggaagaacccacaggaaggcctctataatgagctccagaaggataagatggctgaggcctactccgagatcgggatgaagggcgaaaggagacggggtaaggggcacgacggcctctatcagggtctgagcaccgccaccaaggacacctacgacgccctgcacatgcaggcactgccacctcgg

SEQ ID NO: 59 - DNA, 2096 bp, synthetic construct
  source 1..2096: mol_type = other DNA
tctgtcctcaccccatcttcccttctttcctgcaccgttacgcttactcgcatgtgcattgagtggtgcacgtgcttgaacaaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttcatgatggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 60 - DNA, 2096 bp, synthetic construct
  source 1..2096: mol_type = other DNA
tctgtcctcaccccatcttcccttctttcctgcaccgttacgcttactcgcatgtgcattgagtggtgcacgtgcttgaacaaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttcatgatggcactgcccgtcaccgcactcctgctcccactggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggaggtggtggcagcggaggtggaggatcaggtggaggcggtagcgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 61 - DNA, 2096 bp, synthetic construct
  source 1..2096: mol_type = other DNA
tctgtcctcaccccatcttcccttctttcctgcaccgttacgcttactcgcatgtgcattgagtggtgcacgtgcttgaacaaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttcatgatggctctgccagtgaccgcactgctgctgcccttagccttactccttcacgcagccaggcccgacatccagatgacccagaccaccagctccctttccgcaagcctcggcgacagggtcaccatctcctgtcgggccagccaggacatcagcaagtacctgaactggtaccagcagaagcccgacggcaccgtgaagctgctgatctaccacacctcacggctgcactcaggcgtgccctcacggtttagcggatcaggcagcggcaccgactacagcctgactatcagcaacctggagcaggaggacatcgccacctacttctgccagcagggcaacaccctgccctacaccttcggaggcggcaccaagctggagatcaccggtggcggtggttcaggtggcggaggctcaggaggaggcggcagcgaggtgaagctgcaggagtcaggtccaggactggtggcacccagccagagcctgagcgtgacttgcaccgtgtcaggcgtgagcctgccagactacggcgtgagctggatccggcagcctcctcggaagggcttagagtggctgggcgtgatctggggcagcgagaccacctactacaactcagccctgaagagccggctgaccatcatcaaggacaacagcaagagccaggtgttcctgaagatgaacagcctgcagaccgacgacaccgccatctactactgcgccaagcactactactacggcggcagctacgccatggactactggggacagggtaccagcgtgaccgtgagcagcatcgaggtgatgtaccctcctccctacctggacaacgagaagagcaacggcaccatcatccacgtgaagggcaagcacctgtgccctagccctttattccccggcccctcaaaacccttctgggtgctggtcgtcgtcggtggcgtgctggcatgctacagcctgctggtgaccgtggccttcatcatattctgggtccggtcaaagcggagccggttactgcacagcgactacatgaacatgactccacggcgtccaggtcccactcggaagcactaccaaccctacgctcctccccgtgactttgctgcctaccgtagccgggtgaagttctccaggagcgccgatgccccagcctaccagcagggccagaaccagctctacaatgagcttaaccttggcaggcgggaggagtacgacgtgctggacaagaggaggggccgtgatcccgagatgggaggcaagccccgtaggaagaatccccaggagggcctttacaacgagctccagaaggacaagatggccgaggcctacagcgagatcggcatgaagggagagcgtaggcgtggaaagggccacgacggcctgtaccagggcctgagcactgctaccaaggacacctacgacgccctgcacatgcaggctcttccaccccgg

SEQ ID NO: 62-99 - skipped (38 entries)

SEQ ID NO: 100 - DNA, 1425 bp, synthetic construct
  source 1..1425: mol_type = other DNA
atgggcgtgcctacccagctgctcggtctcctgctgctctggatcaccgacgctatctgcgacatccaaatgacccagagtcccgcttccctcagcacctccctgggtgagaccgtcaccatccagtgccaggcatccgaggacatctacagtggtctcgcctggtaccagcagaagcctggtaagtcccctcagctgctgatctacggtgcttccgatctgcaggacggagtccctagccgcttctcaggctctggctccggtacccagtactccctgaagatcacatccatgcagaccgaagacgagggagtgtacttctgccagcaggggctgacctaccctcggacattcggcggtggaaccaagctcgagctgaagggaggtggaggcagtggtggcggaggatctggtggtggtggctccgaggtccaactgcagcagtccggcgctgagctggtgaggcccggaaccagcgtcaaactcagctgcaaggtgagcggggacaccatcaccttctactacatgcacttcgtcaagcagaggcctgggcagggtcttgaatggatcggccggatcgatccagaggacgagtctacaaagtactccgagaagttcaagaacaaagcaaccctgaccgccgacacaagctccaacaccgcctacctgaagctgtccagcctcacctctgaggacaccgccacctacttctgcatctacggcgggtactacttcgactattggggccaaggggtgatggtcaccgtgtcctctatcgagttcatgtatcctcctccctacctggacaacgagcggagcaacggcaccatcatccacatcaaagagaagcacctctgccacacccaatcctctcccaaactcttctgggccctcgttgtggtcgcaggcgtgctcttctgctacgggctgctggtgactgtggccttgtgcgtgatctggaccaacagtagacggaatcggggaggtcagagcgactacatgaacatgacacctcgcagaccaggcctgacacggaagccctaccaaccatacgctcctgcccgggattttgcagcatatcggccacgggccaagtttagcaggtccgcagagaccgcagccaacctgcaagaccctaaccagctgtacaacgagctgaaccttggtcgccgggaggagtacgacgtcctggagaagaagagagcacgggatcccgagatgggcggaaagcaacaacgccggcggaatcctcaggagggtgtctacaacgccctccagaaggacaagatggctgaggcctactccgagatcggcactaagggcgagcgcagacggggaaagggtcacgacgggctgtaccagggtctcagcaccgcaaccaaggatacctacgacgccctgcacatgcaaaccctcgcaccccgg

SEQ ID NO: 101 - DNA, 1452 bp, synthetic construct
  source 1..1452: mol_type = other DNA
atggctctgcctgtgacagctctgctgctgcctctggctctgcttctgcatgccgccagacctgacatccagatgactcagagccccagcagcctgtctgcctctgtgggagacagagtgacaattacctgccgggccagccaggatgtgaatactgctgtcgcctggtatcaacaaaagcctggcaaggcccctaagctcctgatctacagcgccagctttctgtacagcggcgtgcccagcagattctccggaagcagaagcggcacagatttcacactgaccataagcagcctgcagccagaggatttcgccacctactattgccagcagcactacaccacacctccaacctttggccagggcaccaaggtcgagattaagagaacaggcagcacatctggctctggcaaacctggatctggcgagggctctgaagtccagctggtggaatctggcggaggactggttcaacctggcggctctctgagactgtcttgtgccgcctccggcttcaacatcaaggacacctacatccactgggtccgacaagccccaggcaaaggacttgagtgggtcgccaggatctaccccaccaacggctacaccagatacgccgactctgtgaagggcagattcaccatctctgccgacaccagcaagaataccgcctacctgcagatgaactccctgagagccgaagataccgctgtgtattactgttccagatggggaggcgacggcttctacgccatggatgtttggggccaaggcaccctcgtgaccgtttcttctatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 102 - DNA, 1464 bp, synthetic construct
  source 1..1464: mol_type = other DNA
atggctctgcctgtgacagctctgctgctgcctctggctctgcttctgcatgccgccagacctgacatccagatgactcagagccccagcagcctgtctgcctctgtgggagacagagtgacaattacctgccgggccagccaggatgtgaatactgctgtcgcctggtatcaacaaaagcctggcaaggcccctaagctcctgatctacagcgccagctttctgtacagcggcgtgcccagcagattctccggaagcagaagcggcacagatttcacactgaccataagcagcctgcagccagaggatttcgccacctactattgccagcagcactacaccacacctccaacctttggccagggcaccaaggtcgagattaagagaacaggcagcacatctggctctggcaaacctggatctggcgagggctctgaagtccagctggtggaatctggcggaggactggttcaacctggcggctctctgagactgtcttgtgccgcctccggcttcaacatcaaggacacctacatccactgggtccgacaagccccaggcaaaggacttgagtgggtcgccaggatctaccccaccaacggctacaccagatacgccgactctgtgaagggcagattcaccatctctgccgacaccagcaagaataccgcctacctgcagatgaactccctgagagccgaagataccgctgtgtattactgttccagatggggaggcgacggcttctacgccatggatgtttggggccaaggcaccctcgtgaccgtttcttctaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggctcctctggccggaacatgtggcgttctgctgctgagcctggtcatcaccctgtactgtaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 103 - DNA, 1479 bp, synthetic construct
  source 1..1479: mol_type = other DNA
atggcactcccggtaaccgccttattgcttccccttgccctcttgctccacgcagcacgccccgatatagtcttgactcaatccccacccagtttggcaatgtcattaggcaaacgagcaacaatttcatgtagggcatccgaaagtgtaacgattttggggagtcatttaattcattggtaccaacaaaagcctggacaacccccgacgctcttgatccaattagcatctaacgtccaaaccggagtccccgcacgattctcaggatccggttcccggactgattttacattaactattgatccggtagaggaagatgacgtcgctgtctattattgtcttcaaagtaggacgattccacggacattcggtggcggaactaaattggagattaaaggttccacctctggtagtgggaaacccgggtccggtgaagggtccactaaaggccaaattcaactcgttcaatccggaccagaactgaagaagccaggagaaactgtcaaaataagctgtaaagcttccggttatacatttacagattattccataaattgggtgaaaagggcgccaggaaaagggttaaagtggatgggttggattaatacagagactcgggaacctgcatatgcttatgattttaggggaaggtttgccttttctctggagacttccgcttcaactgcttatctccaaattaataatcttaaatatgaggacacagcaacatacttctgtgctttggactatagttatgctatggattactggggacaaggaaccagtgtcactgtaagttccgctgctgcgacgaccactcctgcaccgcgaccacccactcctgcccctactattgctagtcaaccacttagcttgcgacctgaggcatgtcggcccgcggcaggtggcgcagtccacaccaggggtttagactttgcttgtgatatttatatttgggcaccactcgccgggacttgcggtgttcttctcttgtcccttgttataactctttattgtaagcgcggaaggaagaaattgttatatattttcaaacaaccttttatgcgacccgtacaaacaactcaggaagaggacgggtgttcttgtcggtttccagaagaggaagagggtgggtgtgaactccgggtcaaatttagtaggtcagcagatgcgccggcgtaccaacaaggccaaaaccaactgtataatgaactcaatctcggtaggcgtgaggaatatgatgtccttgataaaaggcgcgggagagatccagaaatgggcggaaaaccacggcgaaagaatccgcaggaagggttatataacgaacttcaaaaggataaaatggctgaagcttattccgaaattggcatgaaaggagagcgacgtaggggcaaagggcatgatggcctttaccaagggctctcaaccgctacaaaagatacttacgacgctttacatatgcaagcacttccacccagg

SEQ ID NO: 104 - DNA, 1476 bp, synthetic construct
  source 1..1476: mol_type = other DNA
atggccctccccgtcacagctctcctgctcccactggcccttcttttgcacgctgctcgccccgatatcgtgctcacccagtcacctccaagccttgccatgagcctcgggaaacgggctaccatctcctgccgggcttcagagtccgtcaccatcctcgggtcacacctcatccactggtaccaacagaaaccagggcagcctcctaccctcttgatccagttggcctccaacgtgcaaactggggttcccgccaggttcagtggctccggatcccggacagatttcacacttaccatcgatcctgtggaggaggacgatgtggccgtctattactgcctgcagtctcgcaccatccctcggaccttcggtggaggcaccaagctcgagatcaagggtagcacctccggctctggaaagccaggctctggtgagggttctaccaagggccaaatccagctggtccagtctgggcccgagctgaagaaacccggggagaccgtgaagatctcctgcaaggcctccggttataccttcaccgactactccatcaactgggtcaagcgcgctcctggaaagggcctcaagtggatgggctggatcaacaccgaaacccgcgagcctgcctatgcttacgacttcaggggccggttcgctttctcactggagacctccgcttccacagcctacctccagatcaacaacctcaagtacgaagacaccgccacctatttctgcgctctcgactattcctacgctatggactactggggtcagggcacctctgtgaccgtctctagcgcagccgccaccacaacaccagccccacggccacctactcccgcacccaccatcgcatcccaaccactcagtctgaggcccgaggcctgtagacctgctgctggaggcgcagtgcatacccgcggtctcgacttcgcctgcgacatctatatctgggccccattggcaggtacctgtggcgtgctgctgctgtcactcgtcatcaccctgtactgccggagtaagcgctctaggctgttgcacagcgactacatgaacatgaccccaagaagaccagggcctacccggaagcactaccagccatacgcacctccccgggactttgccgcctatcggtctcgggtgaagttctcacgctccgctgatgccccagcataccagcaggggcagaaccagctgtacaatgagctcaacctcggtcgccgcgaagagtacgacgtgctcgacaagagaaggggcagggaccctgagatgggaggcaagccccgcagaaagaatccccaggaaggtctgtacaacgagctgcaaaaggataagatggctgaggcctacagcgagatcggcatgaagggcgaaaggagacggggaaagggccacgacgggctctaccagggactctccaccgccaccaaggacacctacgacgccctccacatgcaggctctgccacccagg

SEQ ID NO: 105 - DNA, 1458 bp, synthetic construct
  source 1..1458: mol_type = other DNA
atggcacttcccgtcaccgctctcctgctgcccctcgcactgctgctccatgcagcccgcccagacatcgtcctgacccagtcccctccctccctcgcaatgtccctcgggaaacgggccaccatcagctgccgggcctctgagtcagtgacaatcctcggaagccatctgatccattggtaccagcagaaacccggtcagcctccaaccctcctcatccagctggcctccaacgtgcagacaggagtccccgctcggttctcaggcagcggttccaggaccgacttcaccctgaccatcgaccccgtggaagaggacgatgtggctgtgtactactgcctccagtcccggaccatcccacggaccttcggaggtgggacaaagctggagatcaaaggcagcaccagcggttctggcaagccagggtcaggtgaggggagcacaaagggtcagatccagctggtgcagagcggtcccgagctgaagaagcccggggagaccgttaagatctcctgcaaggctagcgggtacaccttcaccgactatagtatcaactgggtcaagcgcgctcctggcaaggggctcaagtggatggggtggatcaacaccgaaaccagggagcccgcatacgcttatgactttcggggccggttcgccttttccctggagaccagcgcctctaccgcctacctccagatcaacaacctgaagtacgaggacaccgccacctacttctgcgcactcgactactcctacgctatggactactggggtcagggtacctccgtcaccgtctccagcatcgaggtcatgtaccctcctccctacctggacaacgagaagtccaacggcaccatcatccatgtgaagggcaagcatctctgccccagcccactgttccccggaccctctaagcccttctgggtcctggtcgtcgtcggcggtgttctggcttgctacagcttgctggtcaccgtcgccttcatcatcttctgggtgcgctccaagaggagccggctgctgcatagcgactacatgaacatgacccctagaaggcctggtccaacccgcaagcactaccagccttacgcccctccacgggacttcgcagcctaccggtcacgggtgaagttctctcggagcgcagatgccccagcataccagcagggccagaaccagctgtacaacgaacttaaccttggtcggcgggaggaatacgatgtgctggacaagcgcaggggtcgggatcctgaaatgggcgggaaaccacgccggaagaacccacaggaggggctctataacgagctccaaaaggataagatggctgaggcttacagcgagattggaatgaagggagaaagaagacggggcaagggtcacgacgggttgtaccagggtctgagcaccgccaccaaggacacctacgacgccctccacatgcaagcccttccaccccgc

SEQ ID NO: 106 - DNA, 1494 bp, synthetic construct
  source 1..1494: mol_type = other DNA
atggctctccccgtgaccgctctgctgctccctctggccctccttctgcacgcagccagaccacaggtcaagctggaggagtctggtggcggtctggtgcaggcagggaggagcctgaggctgagctgtgcagcttccgagcacacattctcaagccacgtcatggggtggttcagacaggctcccggtaaagagagggagtccgtcgccgtgatcggatggcgggacatctccacctcctacgccgactctgtgaagggccggttcacaatctcacgcgataatgccaagaagacactgtatctgcagatgaattccttgaagcccgaagacaccgccgtctattactgtgctgctagacggatcgacgctgccgacttcgacagctggggacagggtacccaagtgaccgtttcctccggaggcggaggttctggaggaggtgggtcaggtggaggtggctccgaggtgcagctggtcgagtctggcggtggcttggtccaggctggaggcagtctcagactctcctgcgctgcttcagggcggaccttcaccatgggctggttcaggcaggccccaggtaaggagagggagttcgtggccgccatctccctctcccctaccctggcatactacgctgagtccgtgaagggacggtttaccatctcccgggataacgcaaagaacactgtggtcctccaaatgaactccctcaaacccgaggacaccgctctctactattgtgccgcagatcggaagagcgtcatgtccatccggcccgattactggggccaaggcacacaggtgactgtgtccagcacctccaccaccaccccagcaccaaggcctccaacccctgcaccaaccatcgcctcccagccactgtctttgcggccagaagcatgccgcccagcagcaggtggagccgtgcatacaagaggcctggacttcgcctgcgatatctacatctgggctcctctggccggaacatgcggagtcctgctcttgtccctggtgatcaccctgtactgcaagcggggtcggaagaagctcctctacatcttcaagcagcccttcatgagacccgtccagaccacccaggaggaggacgggtgctcatgcaggttccccgaagaggaggagggtggctgtgagctgcgggtgaagttcagcaggtcagcagacgcccctgcctatcagcagggccaaaaccagttgtacaacgagctgaatctggggagacgggaggagtacgatgtccttgacaagagaaggggccgggatccagagatgggcgggaagccaagacggaagaatcctcaggagggtctgtataacgagctgcagaaggacaagatggccgaggcctactccgagatcggcatgaaaggggagcgccgcagaggaaaaggtcacgatggtctgtaccaggggttgagcaccgctaccaaggatacttacgacgctctgcacatgcaagctctgccaccccgg

SEQ ID NO: 107 - DNA, 1491 bp, synthetic construct
  source 1..1491: mol_type = other DNA
atggcactccccgttaccgcccttctgctgcctctcgccttgctgctgcacgcagccagaccacaggtcaagctggaggagtctggtggcgggctcgttcaagcaggtcggagtctccgcctgtcttgcgcagcatcagagcataccttctcctcacacgtgatggggtggttcaggcaagctcccggtaaggagagggagtccgtggccgttatcggctggcgcgatatcagcacctcctacgcagacagcgttaagggccggttcactatctccagggacaacgctaagaagacactctacctccagatgaacagtctgaagcccgaggacaccgcagtgtactattgcgctgctcggcggatcgatgctgccgacttcgacagctggggtcaagggacccaggtcaccgtttccagcggaggtggcggaagtggtggcggaggatcaggtggtggaggctccgaggtccagctggtggaatcaggaggcggcttggtgcaggctggtgggtctttgcggttgtcctgcgcagcttccggcaggaccttcaccatgggatggttcagacaagccccaggtaaggagcgggagtttgtggccgcaatctcactgtctcccaccctcgcttactacgccgagagtgtgaaggggcgcttcacaatcagtcgcgacaacgcaaagaacaccgtcgtcctgcaaatgaactccctgaagcctgaggataccgcactctattactgcgccgccgatcggaagagcgtcatgtccatccggcccgactattggggccaaggcacccaagtgaccgtcagctccacctccacaaccactcccgccccaagaccacctaccccagccccaacaatcgcatcccagcctctgtcccttcggcccgaagcttgtcgccctgcagcaggtggagcagtgcacacccggggactggacttcgcctgcgacatctacatctgggcacccctggctggaacctgcggcgtgttgctgctgagcctggtgatcaccctctactgccgctctaagagaagccggctgctgcatagcgactacatgaacatgacccctaggagaccaggacccacccggaagcactaccagccttacgctcctccacgggatttcgctgcttaccgcagccgggtgaagttttccaggtcagctgacgcccctgcctaccagcagggccagaaccaattgtacaacgaactgaatctgggacggcgcgaggaatacgacgtcctggacaagaggcggggtagagatcccgagatgggcgggaaacctcggcggaagaaccctcaggaggggctctacaacgagctgcagaaggataagatggccgaagcctactccgagatcgggatgaagggtgaacggaggaggggcaagggacacgacggcctgtatcagggcctcagcaccgctaccaaggacacctacgacgccctgcacatgcaggctctcccaccacgg

SEQ ID NO: 108 - DNA, 1476 bp, synthetic construct
  source 1..1476: mol_type = other DNA
atggctcttcccgtcaccgctttgctgctgcccctggcactcctcctccatgctgctcggcctcaggtgaagctggaggagagtggtggcggtctggtgcaagctggcagatctctgcgcctgtcttgcgcagccagcgaacacaccttctcctcccacgtgatggggtggtttcggcaggcacccgggaaagagcgcgagtccgtcgcagtcatcgggtggcgggacatctctaccagctacgcagattccgtcaagggccggttcaccatttcccgggataacgctaagaagaccctctacctgcaaatgaactctctgaagcccgaagacaccgccgtctactattgcgcagcaaggcgcatcgacgctgccgacttcgactcttggggccaaggaacccaggtcaccgtgtcttccggaggaggaggctccggtggtggaggttctggaggtggcggctcagaggtgcagctcgtggagagcggtggtggactcgttcaggcaggcggcagtttgcggctgtcctgtgcagcctccggtcgcactttcactatgggatggttccgccaggctcctggtaaagaaagggagttcgtggccgccatcagtctgagccccaccctcgcatactacgccgagagcgtgaagggtaggttcactatcagccgggacaacgccaagaacaccgtggtgctccagatgaattccctgaagcctgaggataccgccctctactactgcgctgccgaccgcaagagcgtgatgagcatccggcctgactattggggtcaggggacacaggtgaccgtcagcagcatcgaggtgatgtatccaccaccctacctcgacaacgagaagtccaacggcaccatcatccacgtcaaggggaagcacctctgcccttcccctctgttccctggcccctcaaagcccttctgggtcctggtggtggttggtggggtgctggcttgctactccctgctcgtgaccgtggctttcatcatcttctgggttcggagcaaacggtccagactgctgcactccgactacatgaacatgaccccaagaagacctgggcccacacggaagcattaccaaccctatgcaccacctcgggatttcgccgcctacagatcccgggtcaagttctccaggtccgccgatgcaccagcctatcagcaggggcaaaaccagctgtataatgagctgaaccttggacggcgcgaggagtacgacgtgctcgacaaaagacgcggtcgcgacccagagatgggcggcaagcctagacgcaagaatccccaggaggggctctataacgagttgcagaaggataagatggccgaggcctacagcgagatcgggatgaaaggcgaaagacggcgcggaaagggtcacgacggactctaccagggcctgagcacagccaccaaagacacctacgacgctctgcatatgcaagcactgcctccccgg

SEQ ID NO: 109 - DNA, 1095 bp, synthetic construct
  source 1..1095: mol_type = other DNA
atggctctccccgtgaccgctctcctccttcccttggccttgttgctgcacgccgcaaggcctcaggtgaagctggaggaatccggaggcggactcgtccaagcaggtcggtccctcaggctgtcttgcgctgccagcgagcacaccttctctagccacgtgatgggttggttcagacaggccccaggaaaggagcgggaatccgtggcagtgatcggctggcgggacatcagcacctcctacgccgactccgtcaagggccggttcaccatcagccgcgacaacgccaagaagaccctgtacctgcagatgaacagcctcaagcctgaggacaccgccgtgtactactgcgccgccagaaggatcgacgccgcagacttcgactcctggggtcagggaacccaggtgaccgtgtcctccacctctaccaccacaccagcacccagacctcctactcccgctcccaccatcgcttcccagcccctgtccctcagacccgaagcctgcagaccagcagctggcggtgcagtgcacaccaggggtcttgacttcgcctgtgacatctacatctgggctccactggctgggacttgcggcgttctgctgctgagcctggtgatcaccctgtactgcaagcggggccggaagaagctgctctacatcttcaagcagcctttcatgcggcccgttcagaccacccaggaggaagacgggtgcagttgccgcttccctgaggaggaggagggaggatgcgagctgcgggtcaagttctctcggtccgctgatgccccagcctaccagcagggccagaaccagctctataacgagctgaacctcggtaggcgggaggagtacgacgtcctggacaaaaggaggggacgggatcctgagatgggaggaaagccacggcggaagaaccctcaagaggggctgtacaacgaactccagaaggacaagatggctgaggcttattctgagatcggcatgaagggagagcgcagacgcggcaagggacacgatggcctgtatcagggactgagcaccgccaccaaggatacctacgacgccctccatatgcaggctctcccaccacgg

SEQ ID NO: 110 - DNA, 1092 bp, synthetic construct
  source 1..1092: mol_type = other DNA
atggcactgcccgtcacagctctgctgctgcccttggccctcttgttgcacgcagcacggccagaggtgcaacttgtcgagagcggtggtggccttgtgcaggccggaggcagtttgcgcctcagttgtgcagcttctggccggaccttcaccatgggctggttcagacaggcccctggtaaggaacgcgagttcgtggccgccatcagcctgtccccaaccctggcctactacgcagagagcgtgaagggtcggttcaccatctcccgcgacaacgcaaagaacaccgtggtgctccagatgaactccctgaagccagaagacaccgccctgtactactgcgcagccgaccggaagagcgtgatgtccatccgccctgactactggggccaaggcacacaggtcacagtgtccagcacctccactaccactccagctccacgccctccaacacccgcaccaaccatcgccagccagcctctgagtctgagacccgaagcatgccggccagctgctggaggtgccgtgcacaccagagggctggacttcgcctgcgacatctacatctgggctcctctggccggaacttgcggggtgctgctcctctcactggtcatcaccctgtactgcaagaggggcaggaagaagctcctgtacatcttcaagcagcccttcatgcggccagtccagacaacccaggaggaagacggatgcagctgtcgcttccccgaggaggaggaaggcggctgcgaattgcgggtcaagttcagcagatccgctgacgctcctgcctaccaacagggacagaaccagctctacaacgagctgaacctgggaaggcgggaggagtacgacgtcctggacaagagaagaggacgcgaccccgagatgggaggtaagcccagacgcaagaaccctcaagagggactgtataacgagctgcagaaggacaagatggccgaggcctacagcgagatcggcatgaagggtgaaagaagacggggaaaggggcacgacggtctgtatcaggggctctccaccgcaaccaaggatacctatgacgctctgcacatgcaggcactccctccacgc

SEQ ID NO: 111 - DNA, 1077 bp, synthetic construct
  source 1..1077: mol_type = other DNA
atggcactgcccgtgaccgctctgctgctgcctctcgccctgctgctgcatgctgccaggccccaagtcaagctcgaggagtctggtggtgggctcgtccaagctggcaggtccctgagactgagttgtgccgcctccgagcacactttcagctctcacgtgatgggctggttcaggcaggcacccggtaaggagcgggagtctgtggctgttatcgggtggcgggatatctctacctcctacgccgactccgtcaagggccggttcaccatctccagggacaacgcaaagaagaccctctacctccagatgaactcactcaagcccgaggacaccgctgtgtactactgcgcagccagacgcatcgatgccgcagacttcgactcctggggccaaggtacccaagtgacagtgtccagcatcgaggtgatgtacccacctccctacctcgacaacgagaagagcaacggcaccatcatccacgtgaaggggaagcacctgtgtccctctccccttttcccaggaccctccaagccattctgggtcctggtcgttgtcggaggcgtgctcgcttgctattccctgctcgtcaccgtggccttcatcatcttctgggtgcggtccaagagatcccggctgctgcactctgattacatgaacatgacacccaggaggccagggcctaccaggaagcactaccagccctacgctcctccacgcgacttcgcagcataccggtcccgcgtcaagttctcccggtccgcagatgctccagcctatcagcagggccagaaccagctgtacaacgaactcaatttggggaggcgcgaggagtatgatgtgctcgataagagaaggggccgggatcctgagatgggaggcaagcccagacggaagaaccctcaggagggcctgtacaatgagctgcagaaggacaagatggccgaggcctactccgagatcgggatgaagggtgaaagaaggaggggtaaggggcacgacgggctctaccaaggcctgagcaccgctaccaaggacacctacgatgcactgcatatgcaagccctgccaccacgg

SEQ ID NO: 112 - DNA, 1074 bp, synthetic construct
  source 1..1074: mol_type = other DNA
atggccctccctgtcacagccctcttgctgcccctcgcactccttctgcacgcagctcggcctgaggtccagctggtcgagagtggaggcggactggtccaggcaggtgggagtcttcggctttcctgcgcagcttccggacggaccttcaccatgggttggttccggcaggcacctgggaaggagagggagttcgttgccgctatcagcctctcaccaaccctggcctactatgcagagagcgtcaagggccgcttcaccatcagccgcgacaacgccaagaacaccgtcgtgctgcagatgaactccctcaagcccgaggataccgccctgtactactgcgctgccgatcggaagtccgtcatgtccattcggcccgactactggggacagggcacacaggtgaccgtcagcagcatcgaggtcatgtaccctccaccctacctggacaacgagaagagcaacgggaccatcatccacgtgaaggggaagcacctctgtccaagtcccctcttcccaggaccctccaagccattctgggtcctcgtggtggttggaggagtgctcgcctgctactctctgctggtgaccgtcgccttcatcatcttctgggtgcggtccaagcggtctcgcctcctccactccgactacatgaacatgacaccacgcagacctgggcccactaggaagcactatcagccctatgcaccaccccgggatttcgcagcctaccggtcacgggtgaagttcagcagatccgcagacgcaccagcctaccagcaggggcagaaccagctgtataacgagctgaacctcggtcgcagggaggagtacgatgtcctggataagagaaggggcagggatcccgagatgggtggcaagcccagacggaagaatcctcaggaggggctctacaacgagctgcagaaggacaagatggccgaggcttactcagagatcggcatgaaaggggagaggaggcgcggaaaaggccacgacggcctctaccagggactgtccaccgcaaccaaggatacctacgacgccctgcacatgcaagccctcccacctcgg

SEQ ID NO: 113 - DNA, 2232 bp, synthetic construct
  source 1..2232: mol_type = other DNA
atggctctccccgtgaccgctctcctccttcccttggccttgttgctgcacgccgcaaggcctcaggtgaagctggaggaatccggaggcggactcgtccaagcaggtcggtccctcaggctgtcttgcgctgccagcgagcacaccttctctagccacgtgatgggttggttcagacaggccccaggaaaggagcgggaatccgtggcagtgatcggctggcgggacatcagcacctcctacgccgactccgtcaagggccggttcaccatcagccgcgacaacgccaagaagaccctgtacctgcagatgaacagcctcaagcctgaggacaccgccgtgtactactgcgccgccagaaggatcgacgccgcagacttcgactcctggggtcagggaacccaggtgaccgtgtcctccacctctaccaccacaccagcacccagacctcctactcccgctcccaccatcgcttcccagcccctgtccctcagacccgaagcctgcagaccagcagctggcggtgcagtgcacaccaggggtcttgacttcgcctgtgacatctacatctgggctccactggctgggacttgcggcgttctgctgctgagcctggtgatcaccctgtactgcaagcggggccggaagaagctgctctacatcttcaagcagcctttcatgcggcccgttcagaccacccaggaggaagacgggtgcagttgccgcttccctgaggaggaggagggaggatgcgagctgcgggtcaagttctctcggtccgctgatgccccagcctaccagcagggccagaaccagctctataacgagctgaacctcggtaggcgggaggagtacgacgtcctggacaaaaggaggggacgggatcctgagatgggaggaaagccacggcggaagaaccctcaagaggggctgtacaacgaactccagaaggacaagatggctgaggcttattctgagatcggcatgaagggagagcgcagacgcggcaagggacacgatggcctgtatcagggactgagcaccgccaccaaggatacctacgacgccctccatatgcaggctctcccaccacggggatctggcgaaggcagaggatctctgctgacatgcggcgacgtggaagagaaccctggacctatggccctccctgtcacagccctcttgctgcccctcgcactccttctgcacgcagctcggcctgaggtccagctggtcgagagtggaggcggactggtccaggcaggtgggagtcttcggctttcctgcgcagcttccggacggaccttcaccatgggttggttccggcaggcacctgggaaggagagggagttcgttgccgctatcagcctctcaccaaccctggcctactatgcagagagcgtcaagggccgcttcaccatcagccgcgacaacgccaagaacaccgtcgtgctgcagatgaactccctcaagcccgaggataccgccctgtactactgcgctgccgatcggaagtccgtcatgtccattcggcccgactactggggacagggcacacaggtgaccgtcagcagcatcgaggtcatgtaccctccaccctacctggacaacgagaagagcaacgggaccatcatccacgtgaaggggaagcacctctgtccaagtcccctcttcccaggaccctccaagccattctgggtcctcgtggtggttggaggagtgctcgcctgctactctctgctggtgaccgtcgccttcatcatcttctgggtgcggtccaagcggtctcgcctcctccactccgactacatgaacatgacaccacgcagacctgggcccactaggaagcactatcagccctatgcaccaccccgggatttcgcagcctaccggtcacgggtgaagttcagcagatccgcagacgcaccagcctaccagcaggggcagaaccagctgtataacgagctgaacctcggtcgcagggaggagtacgatgtcctggataagagaaggggcagggatcccgagatgggtggcaagcccagacggaagaatcctcaggaggggctctacaacgagctgcagaaggacaagatggccgaggcttactcagagatcggcatgaaaggggagaggaggcgcggaaaaggccacgacggcctctaccagggactgtccaccgcaaccaaggatacctacgacgccctgcacatgcaagccctcccacctcgg

SEQ ID NO: 114 - DNA, 2232 bp, synthetic construct
  source 1..2232: mol_type = other DNA
atggcactgcccgtcacagctctgctgctgcccttggccctcttgttgcacgcagcacggccagaggtgcaacttgtcgagagcggtggtggccttgtgcaggccggaggcagtttgcgcctcagttgtgcagcttctggccggaccttcaccatgggctggttcagacaggcccctggtaaggaacgcgagttcgtggccgccatcagcctgtccccaaccctggcctactacgcagagagcgtgaagggtcggttcaccatctcccgcgacaacgcaaagaacaccgtggtgctccagatgaactccctgaagccagaagacaccgccctgtactactgcgcagccgaccggaagagcgtgatgtccatccgccctgactactggggccaaggcacacaggtcacagtgtccagcacctccactaccactccagctccacgccctccaacacccgcaccaaccatcgccagccagcctctgagtctgagacccgaagcatgccggccagctgctggaggtgccgtgcacaccagagggctggacttcgcctgcgacatctacatctgggctcctctggccggaacttgcggggtgctgctcctctcactggtcatcaccctgtactgcaagaggggcaggaagaagctcctgtacatcttcaagcagcccttcatgcggccagtccagacaacccaggaggaagacggatgcagctgtcgcttccccgaggaggaggaaggcggctgcgaattgcgggtcaagttcagcagatccgctgacgctcctgcctaccaacagggacagaaccagctctacaacgagctgaacctgggaaggcgggaggagtacgacgtcctggacaagagaagaggacgcgaccccgagatgggaggtaagcccagacgcaagaaccctcaagagggactgtataacgagctgcagaaggacaagatggccgaggcctacagcgagatcggcatgaagggtgaaagaagacggggaaaggggcacgacggtctgtatcaggggctctccaccgcaaccaaggatacctatgacgctctgcacatgcaggcactccctccacgcggatctggcgaaggcagaggatctctgctgacatgcggcgacgtggaagagaaccctggacctatggcactgcccgtgaccgctctgctgctgcctctcgccctgctgctgcatgctgccaggccccaagtcaagctcgaggagtctggtggtgggctcgtccaagctggcaggtccctgagactgagttgtgccgcctccgagcacactttcagctctcacgtgatgggctggttcaggcaggcacccggtaaggagcgggagtctgtggctgttatcgggtggcgggatatctctacctcctacgccgactccgtcaagggccggttcaccatctccagggacaacgcaaagaagaccctctacctccagatgaactcactcaagcccgaggacaccgctgtgtactactgcgcagccagacgcatcgatgccgcagacttcgactcctggggccaaggtacccaagtgacagtgtccagcatcgaggtgatgtacccacctccctacctcgacaacgagaagagcaacggcaccatcatccacgtgaaggggaagcacctgtgtccctctccccttttcccaggaccctccaagccattctgggtcctggtcgttgtcggaggcgtgctcgcttgctattccctgctcgtcaccgtggccttcatcatcttctgggtgcggtccaagagatcccggctgctgcactctgattacatgaacatgacacccaggaggccagggcctaccaggaagcactaccagccctacgctcctccacgcgacttcgcagcataccggtcccgcgtcaagttctcccggtccgcagatgctccagcctatcagcagggccagaaccagctgtacaacgaactcaatttggggaggcgcgaggagtatgatgtgctcgataagagaaggggccgggatcctgagatgggaggcaagcccagacggaagaaccctcaggagggcctgtacaatgagctgcagaaggacaagatggccgaggcctactccgagatcgggatgaagggtgaaagaaggaggggtaaggggcacgacgggctctaccaaggcctgagcaccgctaccaaggacacctacgatgcactgcatatgcaagccctgccaccacgg

SEQ ID NO: 115 - DNA, 1479 bp, synthetic construct; identical to SEQ ID NO: 103
  source 1..1479: mol_type = other DNA

SEQ ID NO: 116 - protein, 475 aa, synthetic construct
MGVPTQLLGLLLLWITDAICDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 117 - protein, 484 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 488 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 493 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 492 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 486 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 498 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 497 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 492 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 365 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 364 aa, synthetic construct
MALPVTALLLPLALLLHAARPEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 359 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 358 aa, synthetic construct
MALPVTALLLPLALLLHAARPEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 744 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 744 aa, synthetic construct
MALPVTALLLPLALLLHAARPEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 493 aa, synthetic construct; identical to SEQ ID NO: 119

SEQ ID NO: 132 - DNA, 1452 bp, synthetic construct; identical to SEQ ID NO: 101
  source 1..1452: mol_type = other DNA

SEQ ID NO: 133 - DNA, 1464 bp, synthetic construct; identical to SEQ ID NO: 102
  source 1..1464: mol_type = other DNA

SEQ ID NO: 134 - protein, 484 aa, synthetic construct; identical to SEQ ID NO: 117

SEQ ID NO: 135 - protein, 488 aa, synthetic construct; identical to SEQ ID NO: 118

SEQ ID NO: 136 - DNA, 2001 bp, synthetic construct
  source 1..2001: mol_type = other DNA
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctgtcacgatggctctgcctgtgacagctctgctgctgcctctggctctgcttctgcatgccgccagacctgacatccagatgactcagagccccagcagcctgtctgcctctgtgggagacagagtgacaattacctgccgggccagccaggatgtgaatactgctgtcgcctggtatcaacaaaagcctggcaaggcccctaagctcctgatctacagcgccagctttctgtacagcggcgtgcccagcagattctccggaagcagaagcggcacagatttcacactgaccataagcagcctgcagccagaggatttcgccacctactattgccagcagcactacaccacacctccaacctttggccagggcaccaaggtcgagattaagagaacaggcagcacatctggctctggcaaacctggatctggcgagggctctgaagtccagctggtggaatctggcggaggactggttcaacctggcggctctctgagactgtcttgtgccgcctccggcttcaacatcaaggacacctacatccactgggtccgacaagccccaggcaaaggacttgagtgggtcgccaggatctaccccaccaacggctacaccagatacgccgactctgtgaagggcagattcaccatctctgccgacaccagcaagaataccgcctacctgcagatgaactccctgagagccgaagataccgctgtgtattactgttccagatggggaggcgacggcttctacgccatggatgtttggggccaaggcaccctcgtgaccgtttcttctatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 137 - DNA, 2013 bp, synthetic construct
  source 1..2013: mol_type = other DNA
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctgtcacgatggctctgcctgtgacagctctgctgctgcctctggctctgcttctgcatgccgccagacctgacatccagatgactcagagccccagcagcctgtctgcctctgtgggagacagagtgacaattacctgccgggccagccaggatgtgaatactgctgtcgcctggtatcaacaaaagcctggcaaggcccctaagctcctgatctacagcgccagctttctgtacagcggcgtgcccagcagattctccggaagcagaagcggcacagatttcacactgaccataagcagcctgcagccagaggatttcgccacctactattgccagcagcactacaccacacctccaacctttggccagggcaccaaggtcgagattaagagaacaggcagcacatctggctctggcaaacctggatctggcgagggctctgaagtccagctggtggaatctggcggaggactggttcaacctggcggctctctgagactgtcttgtgccgcctccggcttcaacatcaaggacacctacatccactgggtccgacaagccccaggcaaaggacttgagtgggtcgccaggatctaccccaccaacggctacaccagatacgccgactctgtgaagggcagattcaccatctctgccgacaccagcaagaataccgcctacctgcagatgaactccctgagagccgaagataccgctgtgtattactgttccagatggggaggcgacggcttctacgccatggatgtttggggccaaggcaccctcgtgaccgtttcttctaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggctcctctggccggaacatgtggcgttctgctgctgagcctggtcatcaccctgtactgtaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 138 - DNA, 2028 bp, synthetic construct
  source 1..2028: mol_type = other DNA
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctgtcacgatggcactcccggtaaccgccttattgcttccccttgccctcttgctccacgcagcacgccccgatatagtcttgactcaatccccacccagtttggcaatgtcattaggcaaacgagcaacaatttcatgtagggcatccgaaagtgtaacgattttggggagtcatttaattcattggtaccaacaaaagcctggacaacccccgacgctcttgatccaattagcatctaacgtccaaaccggagtccccgcacgattctcaggatccggttcccggactgattttacattaactattgatccggtagaggaagatgacgtcgctgtctattattgtcttcaaagtaggacgattccacggacattcggtggcggaactaaattggagattaaaggttccacctctggtagtgggaaacccgggtccggtgaagggtccactaaaggccaaattcaactcgttcaatccggaccagaactgaagaagccaggagaaactgtcaaaataagctgtaaagcttccggttatacatttacagattattccataaattgggtgaaaagggcgccaggaaaagggttaaagtggatgggttggattaatacagagactcgggaacctgcatatgcttatgattttaggggaaggtttgccttttctctggagacttccgcttcaactgcttatctccaaattaataatcttaaatatgaggacacagcaacatacttctgtgctttggactatagttatgctatggattactggggacaaggaaccagtgtcactgtaagttccgctgctgcgacgaccactcctgcaccgcgaccacccactcctgcccctactattgctagtcaaccacttagcttgcgacctgaggcatgtcggcccgcggcaggtggcgcagtccacaccaggggtttagactttgcttgtgatatttatatttgggcaccactcgccgggacttgcggtgttcttctcttgtcccttgttataactctttattgtaagcgcggaaggaagaaattgttatatattttcaaacaaccttttatgcgacccgtacaaacaactcaggaagaggacgggtgttcttgtcggtttccagaagaggaagagggtgggtgtgaactccgggtcaaatttagtaggtcagcagatgcgccggcgtaccaacaaggccaaaaccaactgtataatgaactcaatctcggtaggcgtgaggaatatgatgtccttgataaaaggcgcgggagagatccagaaatgggcggaaaaccacggcgaaagaatccgcaggaagggttatataacgaacttcaaaaggataaaatggctgaagcttattccgaaattggcatgaaaggagagcgacgtaggggcaaagggcatgatggcctttaccaagggctctcaaccgctacaaaagatacttacgacgctttacatatgcaagcacttccacccagg

SEQ ID NO: 139-199 - skipped (61 entries)

SEQ ID NO: 200 - DNA, 23 bp, synthetic construct
  source 1..23: mol_type = other DNA
caaacaccattgtcacactccaa

SEQ ID NO: 201 - protein, 493 aa, synthetic construct
MALPVTALLLPLALLLHAARPDIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 202 - protein, 207 aa, synthetic construct
KNQVEQSPQSLIILEGKNCTLQCNYTVSPFSNLRWYKQDTGRGPVSLTIMTFSENTKSNGRYTATLDADTKQSSLHITASQLSDSASYICVVNHSGGSYIPTFGRGTSLIVHPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 203 - protein, 246 aa, synthetic construct
DVKVTQSSRYLVKRTGEKVFLECVQDMDHENMFWYRQDPGLGLRLIYFSYDVKMKEKGDIPEGYSVSREKKERFSLILESASTNQTSMYLCASSFLMTSGDPYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 204 - protein, 115 aa, synthetic construct
MQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPY

SEQ ID NO: 205 - protein, 112 aa, synthetic construct
MGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVL

SEQ ID NO: 206 - protein, 166 aa, synthetic construct
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 207 - protein, 452 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 208 - protein, 1462 aa, synthetic construct
LSVKAQTAHIVLEDGTKMKGYSFGHPSSVAGEVVFNTGLGGYPEAITDPAYKGQILTMANPIIGNGGAPDTTALDELGLSKYLESNGIKVSGLLVLDYSKDYNHWLATKSLGQWLQEEKVPAIYGVDTRMLTKIIRDKGTMLGKIEFEGQPVDFVDPNKQNLIAEVSTKDVKVYGKGNPTKVVAVDCGIKNNVIRLLVKRGAEVHLVPWNHDFTKMEYDGILIAGGPGNPALAEPLIQNVRKILESDRKEPLFGISTGNLITGLAAGAKTYKMSMANRGQNQPVLNITNKQAFITAQNHGYALDNTLPAGWKPLFVNVNDQTNEGIMHESKPFFAVQFHPEVTPGPIDTEYLFDSFFSLIKKGKATTITSVLPKPALVASRVEVSKVLILGSGGLSIGQAGEFDYSGSQAVKAMKEENVKTVLMNPNIASVQTNEVGLKQADTVYFLPITPQFVTEVIKAEQPDGLILGMGGQTALNCGVELFKRGVLKEYGVKVLGTSVESIMATEDRQLFSDKLNEINEKIAPSFAVESIEDALKAADTIGYPVMIRSAYALGGLGSGICPNRETLMDLSTKAFAMTNQILVEKSVTGWKEIEYEVVRDADDNCVTVCNMENVDAMGVHTGDSVVVAPAQTLSNAEFQMLRRTSINVVRHLGIVGECNIQFALHPTSMEYCIIEVNARLSRSSALASKATGYPLAFIAAKIALGIPLPEIKNVVSGKTSACFEPSLDYMVTKIPRWDLDRFHGTSSRIGSSMKSVGEVMAIGRTFEESFQKALRMCHPSIEGFTPRLPMNKEWPSNLDLRKELSEPSSTRIYAIAKAIDDNMSLDEIEKLTYIDKWFLYKMRDILNMEKTLKGLNSESMTEETLKRAKEIGFSDKQISKCLGLTEAQTRELRLKKNIHPWVKQIDTLAAEYPSVTNYLYVTYNGQEHDVNFDDHGMMVLGCGPYHIGSSVEFDWCAVSSIRTLRQLGKKTVVVNCNPETVSTDFDECDKLYFEELSLERILDIYHQEACGGCIISVGGQIPNNLAVPLYKNGVKIMGTSPLQIDRAEDRSIFSAVLDELKVAQAPWKAVNTLNEALEFAKSVDYPCLLRPSYVLSGSAMNVVFSEDEMKKFLEEATRVSQEHPVVLTKFVEGAREVEMDAVGKDGRVISHAISEHVEDAGVHSGDATLMLPTQTISQGAIEKVKDATRKIAKAFAISGPFNVQFLVKGNDVLVIECNLRASRSFPFVSKTLGVDFIDVATKVMIGENVDEKHLPTLDHPIIPADYVAIKAPMFSWPRLRDADPILRCEMASTGEVACFGEGIHTAFLKAMLSTGFKIPQKGILIGIQQSFRPRFLGVAEQLHNEGFKLFATEATSDWLNANNVPATPVAWPSQEGQNPSLSSIRKLIRDGSIDLVINLPNNNTKFVHDNYVIRRTAVDSGIPLLTNFQVTKLFAEAVQKSRKVDSKSLFHYRQYSAGKAA

SEQ ID NO: 209 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 210 - protein, 1353 aa, synthetic construct
AAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 211 - protein, 431 aa, synthetic construct
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 212 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 213 - protein, 133 aa, synthetic construct [sequence omitted]

(19) United States
(12) Patent Application Publication
(10) Pub. No.: US 2025/0262324 A1
(43) Pub. Date: Aug. 21, 2025

(54) Circular RNA Compositions

(71) Applicant: Orna Therapeutics, Inc., Watertown, MA (US)

(72) Inventors: Amy M. Becker, Lexington, MA (US); Robert Alexander Wesselhoeft, IV, Somerville, MA (US); Akinola O. Emmanuel, Cambridge, MA (US); Jui Dutta-Simmons, Sudbury, MA (US); Allen T. Horhota, Westford, MA (US); Kevin J. Kauffman, Newton, MA (US); Bun Chau, Needham, MA (US); Thomas Lee, Watertown, MA (US)

(73) Assignee: Orna Therapeutics, Inc., Watertown, MA (US)

(21) Appl. No.: 19/200,761

(22) Filed: May 7, 2025

Related U.S. Application Data
(63) Continuation of application No. PCT/US23/78875, filed on Nov. 7, 2023.
(60) Provisional application No. 63/509,361, filed on Jun. 21, 2023.
(60) Provisional application No. 63/501,820, filed on May 12, 2023.
(60) Provisional application No. 63/423,760, filed on Nov. 8, 2022.

(51) Int. Cl. A61K 48/00; A61K 9/1272; A61K 9/51; A61K 40/11; A61K 40/15; A61K 40/17; A61K 40/31; A61K 40/42; A61P 35/00; C07K 16/28; C07K 16/32; C12N 15/85; C12N 15/88

(52) U.S. Cl. CPC A61K 48/005; A61K 9/1272; A61K 9/5123; A61K 40/11; A61K 40/15; A61K 40/17; A61K 40/31; A61K 40/4205; A61K 40/4211; A61P 35/00; C07K 16/2803; C07K 16/32; C12N 15/85; C12N 15/88; A61K 2239/13; A61K 2239/17; A61K 2239/21; C07K 2317/565; C07K 2317/622; C12N 2800/22; C12N 2830/50; C12N 2840/203

(57) ABSTRACT

Provided herein are circular RNA constructs comprising an IRES, and at least one expression sequence encoding binding molecule, compositions thereof, and methods of treatment, including for cancer and autoimmune disease. In particular, circular RNA comprising an IRES and a CD19 binder, a HER2 binder, or a BCMA binder are provided, optionally formulated with a delivery vehicle. Precursor polynucleotides comprising an IRES, and at least one expression sequence encoding a CAR construct are also described herein.

### CROSS-REFERENCE TO RELATED APPLICATIONS

This application is a continuation of International Application No. PCT/US2023/078875, filed Nov. 7, 2023, which claims the benefit of priority of U.S. Provisional Application No. 63/423,760, filed Nov. 8, 2022, U.S. Provisional Application No. 63/501,820, filed May 12, 2023, and U.S. Provisional Application No. 63/509,361, filed Jun. 21, 2023, each of which is incorporated by reference herein in its entirety for any purpose.

### SEQUENCE LISTING

This application is filed with a Sequence Listing in electronic format. The Sequence Listing is provided as a file entitled “01318-0002-00PCT_SL.xml” created on Oct. 31, 2023, which is 213,220 bytes in size. The information in the electronic format of the sequence listing is incorporated herein by reference in its entirety.

### INTRODUCTION AND BACKGROUND

Circular RNA (circRNA or oRNA™) is a known stable form of RNA that provides an advantage compared to linear RNA in structure and function, especially in the case of molecules that are prone to folding in an inactive conformation (Wang and Ruffner, 1998). Circular RNA polynucleotides lack the free ends necessary for exonuclease-mediated degradation, causing them to be resistant to several mechanisms of RNA degradation and granting extended half-lives when compared to an equivalent linear RNA. Circularization may allow for the stabilization of RNA polynucleotides that generally suffer from short half-lives and may improve the overall efficacy of exogenous mRNA in a variety of applications. Circular RNA can also be particularly interesting and useful for in vivo applications, especially in the research area of RNA-based control of gene expression and therapeutics, including protein replacement therapy and vaccination.

Adoptive T-cell immunotherapy is a rapidly growing field, in particular in cancer treatments. In general, chimeric antigen receptor (CAR) T cell or “CAR-T” engagement of CD19-expressing cancer cells results in T-cell activation, proliferation and secretion of inflammatory cytokines and chemokines resulting in tumor cell lysis. However, while CAR-T therapies have become an important tool in cancer treatments, they have toxic side effects and involve complex procedures. Treatment with CAR-T can lead to a large and rapid release of cytokines into the blood and can cause cytokine release syndrome (CRS) or CAR-T cell-related encephalopathy syndrome (CRES), also referred to as neurotoxicity associated with CAR-T. CRS is the most common and well-described toxicity associated with CAR-T therapy, occurring in over 90% of patients at any grade and is characterized by high fever, hypotension, hypoxia and/or multiple organ toxicity and can lead to death. Neurotoxicity is characterized by damage to nervous tissue that can cause tremors, encephalopathy, dizziness or seizures. Additionally, prior to infusion, the patients generally undergo lymphodepletion. Lymphodepletion is known to increase CAR-T cell expansion and enhanced efficacy of infused CAR-T cells by, for example, altering the tumor phenotype and microenvironment. However, lymphodepletion agents often cause side effects to the patients. For example, lymphodepletion can cause neutropenia, anemia, thrombocytopenia, and immunosuppression, leading to a greater risk of infection, along with other toxicities. In addition to the toxicities associated with targeted CAR-T therapies, there are procedures, specialized equipment, and costs involved in producing the modified lymphocytes. CAR-T therapies require an assortment of protocols to isolate, genetically modify, and selectively expand the redirected cells before infusing them back into the patient.

In a compassionate-use anti-CD19 CAR T cell therapy for refractory systemic lupus erythematosus, autologous T cells from five SLE patients “were transduced with a lentiviral anti-CD19 CAR vector, expanded and reinfused . . . into the patients after lymphodepletion with fludarabine and cyclophosphamide. CAR T cells expanded in vivo led to deep depletion of B cells, improvement of clinical symptoms and normalization of laboratory parameters including seroconversion of anti-double-stranded DNA antibodies. Remission of SLE according to DORIS criteria was achieved in all five patients after 3 months and the median (range) Systemic Lupus Erythematosus Disease Activity Index score after 3 months was 0 (2).” See Mackensen et al., Anti-CD19 CAR T cell therapy for refractory systemic lupus erythematosus, Nature Medicine (2022); see also Nunez et al., Cytokine and reactivity profiles in SLE patients following anti-CD19 CART therapy, Molecular Therapy (2023).

Because circRNAs are more stable and can be expressed in tissue-specific manner, and because using circRNAs can avoid the lymphodepletion step of traditional therapies, circRNAs provide an attractive alternative to traditional CAR therapies and other therapies. Accordingly, provided herein are circular RNA constructs that comprise an internal ribosome entry site (IRES) and at least one expression sequence encoding a binding molecule. In certain embodiments, the binding molecule encodes a CAR that targets a cancer antigen, for use in treating cancer. The circular RNA can be formulated with a transfer vehicle to facilitate and/or enhance the delivery and release of circRNA to one or more target cells. Accordingly, lipid nanoparticles (LNPs) or other transfer vehicles containing ionizable lipids may be used to deliver the circular RNA described herein, for example, to a patient in need of treatment.

### SUMMARY

The present disclosure provides circular RNAs that encode cancer-binding polypeptides paired with lipid transfer vehicles for use in treating cancer. In particular, the present disclosure provides circular RNA comprising an IRES and a nucleic acid encoding a binding molecule, wherein the IRES and the nucleic acid encoding the binding molecule are paired for optimal expression of the polypeptide binding molecule. It has surprisingly been found that certain IRESes and nucleic acid combinations work better than others for optimal expression. It has further been discovered that certain transfer vehicles may work better with certain IRES/binding molecule combinations. Thus, particularly preferred combinations of IRES/nucleic acid encoding binding molecule/transfer vehicles are provided herein. In some embodiments, the circular RNAs provided herein may be used in treating or preventing cancer. In some embodiments, the circular RNAs provided herein may be used in treating or preventing an autoimmune disease, e.g., a B cell mediated autoimmune disease, e.g., lupus.

In some embodiments engineered chimeric antigen receptors (CARs) are encoded by the circular RNA and may be inserted into and expressed by immune cells, including T cells, NK cells, macrophages, etc., via engineered circular RNAs (circRNAs or oRNAs) after delivery via a lipid transfer vehicle. In some embodiments, the CAR may recognize a specific antigen (e.g., CD19, HER2, or BCMA) and, when bound to that antigen, activate the immune cell to attack and destroy the cell. The circular RNAs, compositions, and methods herein are thus useful for reducing known side effects associated with CAR-T therapies by programming circulating immune cells, e.g., T cells, with tumor-recognizing capabilities and by using lipid transfer vehicles (e.g., LNPs) to deliver the circular RNA constructs that can efficiently introduce the CAR genes to the immune cells. Methods directed to the manufacture of such circularized RNA constructs, along with methods of treating a subject in need using the circular RNA are also provided. Linear precursor RNA polynucleotides are provided for producing circular RNA constructs, that comprises a core functional element comprising a translation initiation element (TIE). The TIE can comprise an untranslated region (UTR), aptamer complex or a combination thereof. The UTR can be in whole or in part from a viral or eukaryotic mRNA. The UTR can comprise a viral or eukaryotic internal ribosome entry site (IRES). Pharmaceutical compositions are also provided for the linear precursor and circular RNA constructs comprising an IRES, an expression sequence, and optionally a transfer vehicle. In certain embodiments, the circular RNA constructs comprise an expression sequence encoding a CAR construct targeting a cancer antigen. The pharmaceutical compositions of the present disclosure are particularly suitable for efficient protein expression in immune cells in vivo. The transfer vehicles can comprise, e.g., ionizable lipids, PEG-modified lipids, helper lipids, and/or structural lipids, that are capable of encapsulating the circular RNAs.

Accordingly, the following embodiments are provided:

Embodiment 1. A circular RNA construct comprising:
(A) an IRES comprising a sequence that is at least 80% identical to a sequence selected from any one of SEQ ID NOs: 1-18, and
(B) at least one expression sequence encoding a binding molecule.

Embodiment 2. A circular RNA construct comprising:
(A) an IRES comprising a sequence that is at least 80% identical to a sequence selected from any one of SEQ ID NOs: 1-18, and
(B) at least one expression sequence encoding a chimeric antigen receptor (CAR) targeting a cancer antigen.

Embodiment 3. A circular RNA construct comprising:
(A) an IRES comprising a sequence that is at least 80% identical to a sequence selected from any one of SEQ ID NOs: 1-18, and
(B) at least one expression sequence encoding a CAR targeting a cancer antigen, wherein the CAR construct comprises a CD19 binder.

Embodiment 4. The circular RNA construct of embodiment 3, wherein the expression sequence comprises a sequence that is at least 80% identical to a sequence selected from any one of SEQ ID NOs: 19-34.

Embodiment 5. A circular RNA construct comprising:
(A) an IRES comprising a sequence that is at least 80% identical to a sequence selected from any one of SEQ ID NOs: 1-18, and
(B) at least one expression sequence encoding a CAR targeting a cancer antigen, wherein the CAR construct comprises a BCMA binder.

Embodiment 6. The circular RNA construct of embodiment 5, wherein the expression sequence comprises a sequence that is at least 80% identical to a sequence selected from any one of SEQ ID NOs: 103-115.

Embodiment 7. A circular RNA construct comprising:
(A) an IRES selected from an Enterovirus, Kobuvirus, Parechovirus, Hunnivirus, Passerivirus, Mischivirus, and Cardiovirus, and
(B) at least one expression sequence encoding a binding molecule.

Embodiment 8. A circular RNA construct comprising:
(A) an IRES selected from an Enterovirus, Kobuvirus, Parechovirus, Hunnivirus, Passerivirus, Mischivirus, and Cardiovirus, and
(B) at least one expression sequence encoding a chimeric antigen receptor (CAR) targeting a cancer antigen.

Embodiment 9. A circular RNA construct comprising:
(A) an IRES selected from an Enterovirus, Kobuvirus, Parechovirus, Hunnivirus, Passerivirus, Mischivirus, and Cardiovirus, and
(B) at least one expression sequence encoding a CAR targeting a cancer antigen, wherein the CAR construct comprises a CD19 binder.

Embodiment 10. The circular RNA construct of embodiment 9, wherein the expression sequence comprises a sequence that is at least 80% identical to a sequence selected from any one of SEQ ID NOs: 19-34.

Embodiment 11. A circular RNA construct comprising:
(A) an IRES selected from an Enterovirus, Kobuvirus, Parechovirus, Hunnivirus, Passerivirus, Mischivirus, and Cardiovirus, and
(B) at least one expression sequence encoding a CAR targeting a cancer antigen, wherein the CAR construct comprises a BCMA binder.

Embodiment 12. The circular RNA construct of embodiment 11, wherein the expression sequence comprises a sequence that is at least 80% identical to a sequence selected from any one of SEQ ID NOs: 103-115.

Embodiment 13. A pharmaceutical composition comprising:
(A) a circular RNA construct comprising:
   i. an IRES comprising a sequence that is at least 80% identical to a sequence selected from any one of SEQ ID NOs: 1-18, and
   ii. at least one expression sequence encoding a binding molecule, and
(B) a transfer vehicle.

Embodiment 14. A pharmaceutical composition comprising:
(A) a circular RNA construct comprising:
   i. an IRES comprising a sequence that is at least 80% identical to a sequence selected from any one of SEQ ID NOs: 1-18, and
   ii. at least one expression sequence encoding a CAR targeting a cancer antigen, and
(B) a transfer vehicle.

Embodiment 15. A pharmaceutical composition comprising:
(A) a circular RNA construct comprising:
   i. an IRES selected from an Enterovirus, Kobuvirus, Parechovirus, Hunnivirus, Passerivirus, Mischivirus, and Cardiovirus, and
   ii. at least one expression sequence encoding a binding molecule, and
(B) a transfer vehicle.

Embodiment 16. A pharmaceutical composition comprising:
(A) a circular RNA construct comprising:
   i. an IRES selected from an Enterovirus, Kobuvirus, Parechovirus, Hunnivirus, Passerivirus, Mischivirus, and Cardiovirus, and
   ii. at least one expression sequence encoding a CAR targeting a cancer antigen, and
(B) a transfer vehicle.

Embodiment 17. A pharmaceutical composition comprising:
(A) a circular RNA construct comprising:
   i. an IRES comprising a sequence that is at least 80% identical to a sequence selected from any one of SEQ ID NOs: 1-18, and
   ii, at least one expression sequence encoding a binding molecule, and
(B) a transfer vehicle comprising an ionizable lipid.

Embodiment 18. A pharmaceutical composition comprising:
(A) a circular RNA construct comprising:
   i. an IRES comprising a sequence that is at least 80% identical to a sequence selected from any one of SEQ ID NOs: 1-18, and
   ii. at least one expression sequence encoding a CAR targeting a cancer antigen, and
(B) a transfer vehicle comprising an ionizable lipid.

Embodiment 19. A pharmaceutical composition comprising:
(A) a circular RNA construct comprising:
   i. an IRES comprising a sequence that is at least 80% identical to a sequence selected from any one of SEQ ID NOs: 1-18, and
   ii. at least one expression sequence encoding a CAR targeting a cancer antigen, wherein the CAR construct comprises a CD19 binder, and
(B) a transfer vehicle comprising an ionizable lipid.

Embodiment 20. The pharmaceutical composition of embodiment 19, wherein the expression sequence comprises a sequence that is at least 80% identical to a sequence selected from any one of SEQ ID NOs: 19-34.

Embodiment 21. A pharmaceutical composition comprising:
(A) a circular RNA construct comprising:
   i. an IRES comprising a sequence that is at least 80% identical to a sequence selected from any one of SEQ ID NOs: 1-18, and
   ii. at least one expression sequence encoding a CAR targeting a cancer antigen, wherein the CAR construct comprises a BMCA binder, and
(B) a transfer vehicle comprising an ionizable lipid.

Embodiment 22. The pharmaceutical composition of embodiment 21, wherein the expression sequence comprises a sequence that is at least 80% identical to a sequence selected from any one of SEQ ID NOs: 103-115.

Embodiment 23. A pharmaceutical composition comprising:
(A) a circular RNA construct comprising:
   i. an IRES selected from an Enterovirus, Kobuvirus, Parechovirus, Hunnivirus, Passerivirus, Mischivirus, and Cardiovirus, and
   ii. at least one expression sequence encoding a binding molecule, and
(B) a transfer vehicle comprising an ionizable lipid.

Embodiment 24. A pharmaceutical composition comprising:
(A) a circular RNA construct comprising:
   i. an IRES selected from an Enterovirus, Kobuvirus, Parechovirus, Hunnivirus, Passerivirus, Mischivirus, and Cardiovirus, and
   ii. at least one expression sequence encoding a CAR targeting a cancer antigen, and
(B) a transfer vehicle comprising an ionizable lipid.

Embodiment 25. A pharmaceutical composition comprising:
(A) a circular RNA construct comprising:
   i. an IRES selected from an Enterovirus, Kobuvirus, Parechovirus, Hunnivirus, Passerivirus, Mischivirus, and Cardiovirus, and
   ii. at least one expression sequence encoding a CAR targeting a cancer antigen, wherein the CAR construct comprises a CD19 binder, and
(B) a transfer vehicle comprising an ionizable lipid.

Embodiment 26. The pharmaceutical composition of embodiment 25, wherein the expression sequence comprises a sequence that is at least 80% identical to a sequence selected from any one of SEQ ID NOs: 19-34.

Embodiment 27. A pharmaceutical composition comprising:
(A) a circular RNA construct comprising:
   i. an IRES selected from an Enterovirus, Kobuvirus, Parechovirus, Hunnivirus, Passerivirus, Mischivirus, and Cardiovirus, and
   ii. at least one expression sequence encoding a CAR targeting a cancer antigen, wherein the CAR construct comprises a BMCA binder, and
(B) a transfer vehicle comprising an ionizable lipid.

Embodiment 28. The pharmaceutical composition of embodiment 27, wherein the expression sequence comprises a sequence that is at least 80% identical to a sequence selected from any one of SEQ ID NOs: 103-115.

Embodiment 29. A pharmaceutical composition comprising:
(A) a circular RNA construct comprising:
   i. an IRES comprising a sequence that is at least 80% identical to a sequence selected from any one of SEQ ID NOs: 1-18, and
   ii. at least one expression sequence encoding a binding molecule, and
(B) a transfer vehicle comprising:
   (i) an ionizable lipid of Formula (I)

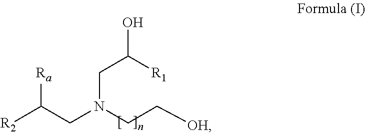

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; or
   (ii) an ionizable lipid of Formula (II)

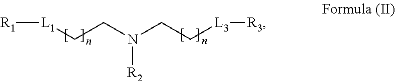

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

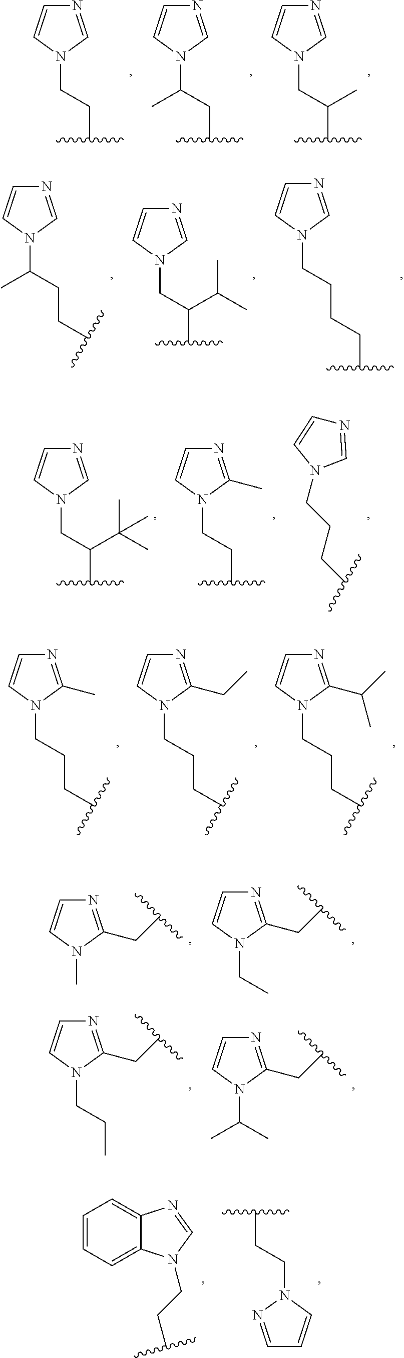

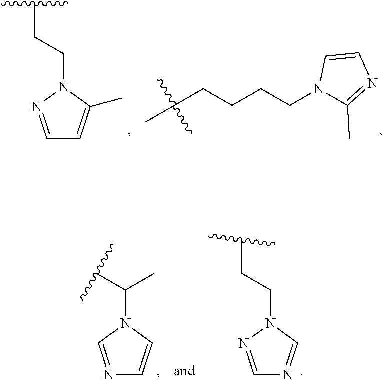

Embodiment 30. A pharmaceutical composition comprising:
(A) a circular RNA construct comprising:
   i. an IRES comprising a sequence that is at least 80% identical to a sequence selected from any one of SEQ ID NOs: 1-18, and
   ii. at least one expression sequence encoding a CAR targeting a cancer antigen, and
(B) a transfer vehicle comprising:
   (i) an ionizable lipid of Formula (I)

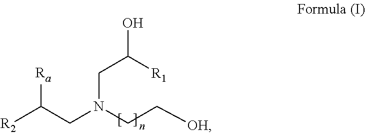

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
   (ii) an ionizable lipid of Formula (II)

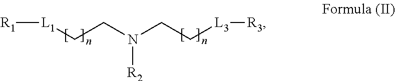

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₀-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

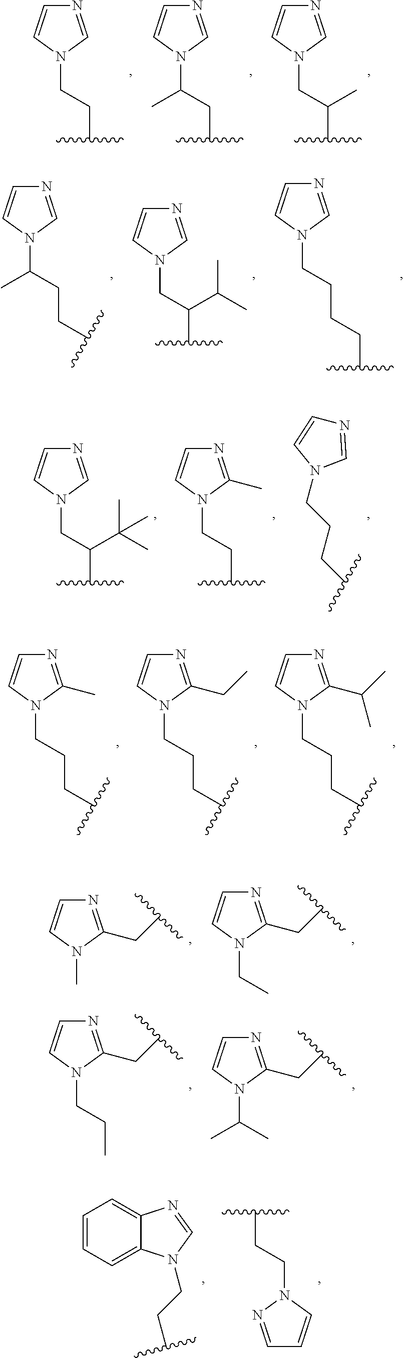

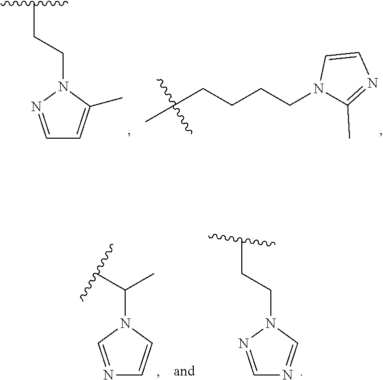

Embodiment 31. A pharmaceutical composition comprising:
(A) a circular RNA construct comprising:
   i. an IRES comprising a sequence that is at least 80% identical to a sequence selected from any one of SEQ ID NOs: 1-18, and
   ii. at least one expression sequence encoding a CAR construct targeting a cancer antigen, wherein the CAR construct comprises a CD19 binder, and
(B) a transfer vehicle comprising:
(i) an ionizable lipid of Formula (I)

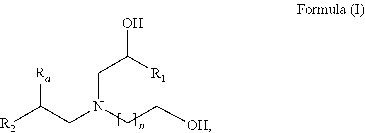

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
   (ii) an ionizable lipid of Formula (II)

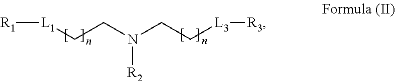

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

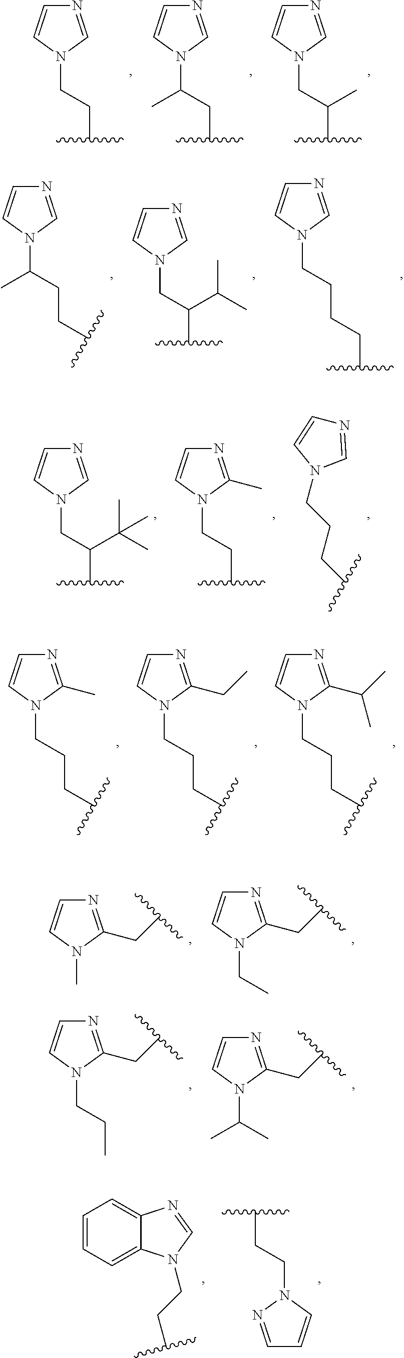

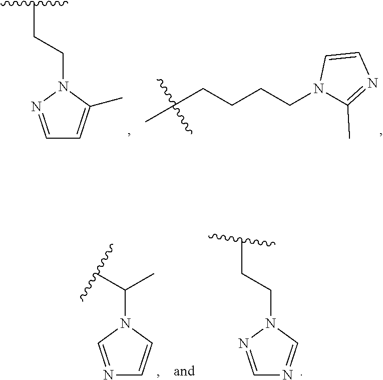

Embodiment 32. A pharmaceutical composition comprising:
(A) a circular RNA construct comprising:
   i. an IRES comprising a sequence that is at least 80% identical to a sequence selected from any one of SEQ ID NOs: 1-18, and
   ii. at least one expression sequence encoding a CAR construct targeting a cancer antigen, wherein the CAR construct comprises a CD19 binder, and wherein the expression sequence comprises a sequence that is at least 80% identical to a sequence selected from any one of SEQ ID NOs: 19-34, and
(B) a transfer vehicle comprising:
(i) an ionizable lipid of Formula (I)

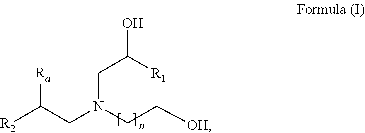

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
(ii) an ionizable lipid of Formula (II)

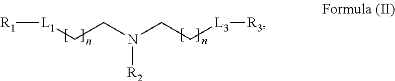

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

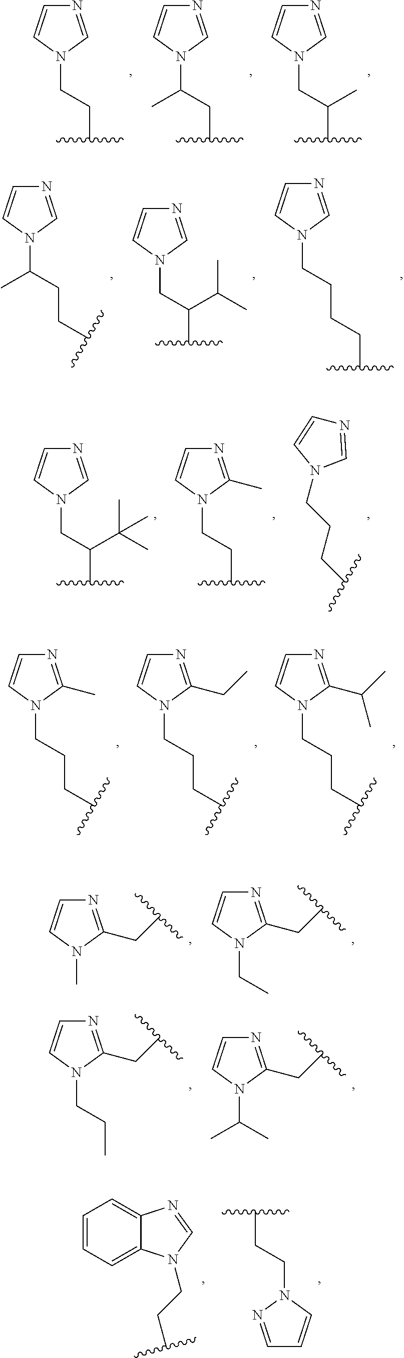

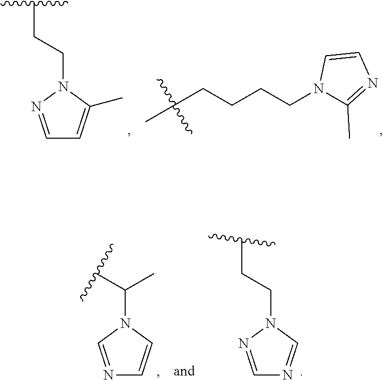

Embodiment 33. A pharmaceutical composition comprising:
(A) a circular RNA construct comprising:
   i. an IRES comprising a sequence that is at least 80% identical to a sequence selected from any one of SEQ ID NOs: 1-18, and
   ii. at least one expression sequence encoding a CAR construct targeting a cancer antigen, wherein the CAR construct comprises a BCMA binder, and
(B) a transfer vehicle comprising:
(i) an ionizable lipid of Formula (I)

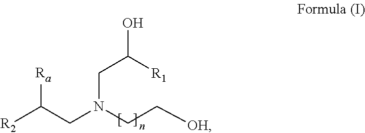

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
(ii) an ionizable lipid of Formula (II)

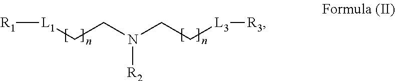

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfoncalkyl; and
R₂ is selected from a group consisting of:

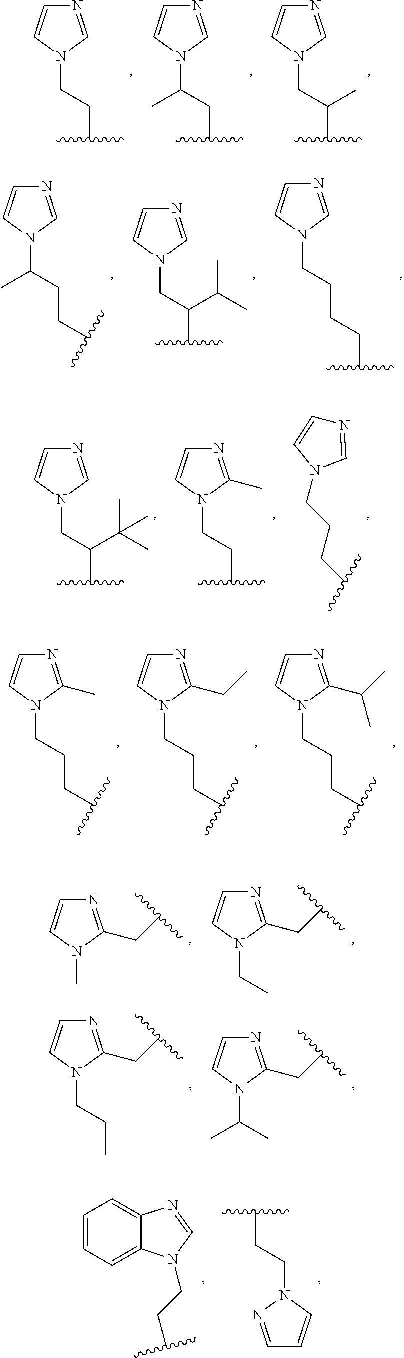

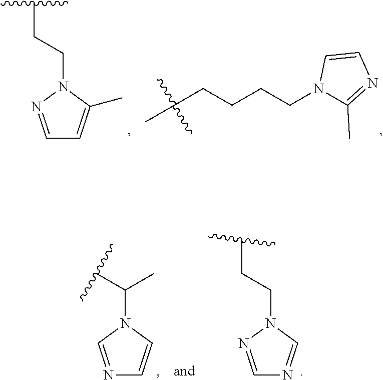

Embodiment 34. A pharmaceutical composition comprising:
(A) a circular RNA construct comprising:
   i. an IRES comprising a sequence that is at least 80% identical to a sequence selected from any one of SEQ ID NOs: 1-18, and
   ii. at least one expression sequence encoding a CAR construct targeting a cancer antigen, wherein the CAR construct comprises an anti-BCMA binder, wherein the expression sequence comprises a sequence that is at least 80% identical to a sequence selected from any one of SEQ ID NOs: 103-115, and
(B) a transfer vehicle comprising:
(i) an ionizable lipid of Formula (I)

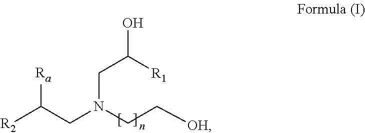

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
(ii) an ionizable lipid of Formula (II)

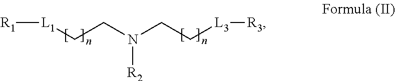

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

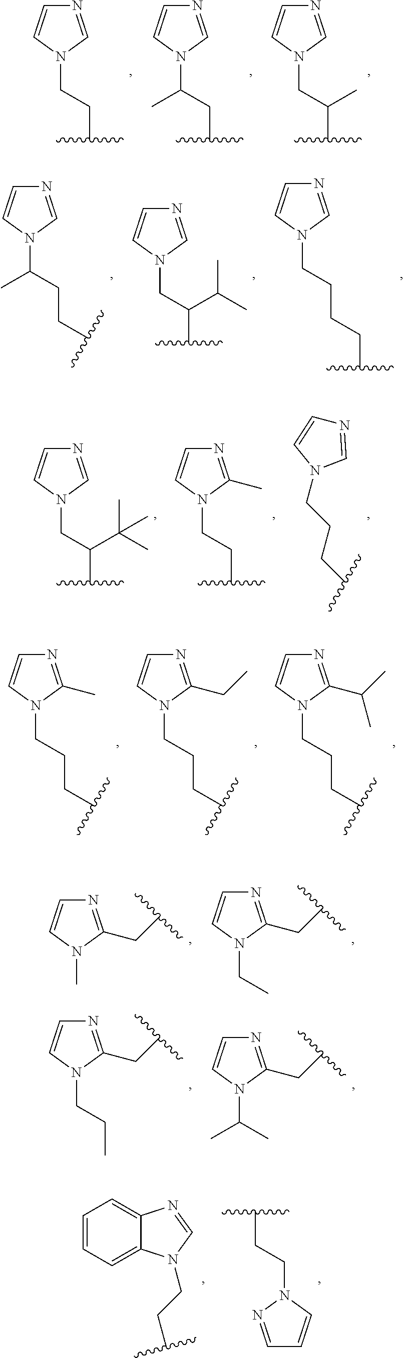

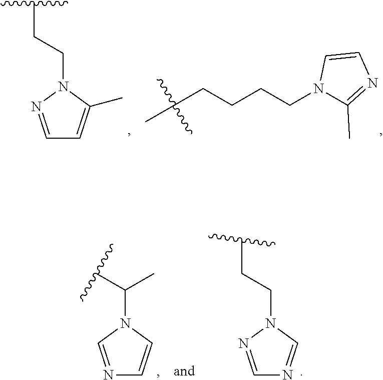

Embodiment 35. A pharmaceutical composition comprising:
(A) a circular RNA construct comprising:
   i. an IRES selected from an Enterovirus, Kobuvirus, Parechovirus, Hunnivirus, Passerivirus, Mischivirus, and Cardiovirus, and
   ii. at least one expression sequence encoding a binding molecule, and
(B) a transfer vehicle comprising:
(i) an ionizable lipid of Formula (I)

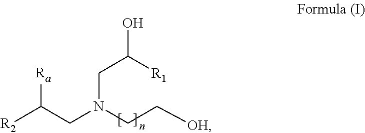

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
(ii) an ionizable lipid of Formula (II)

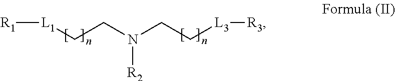

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfoncalkyl; and
R₂ is selected from a group consisting of:

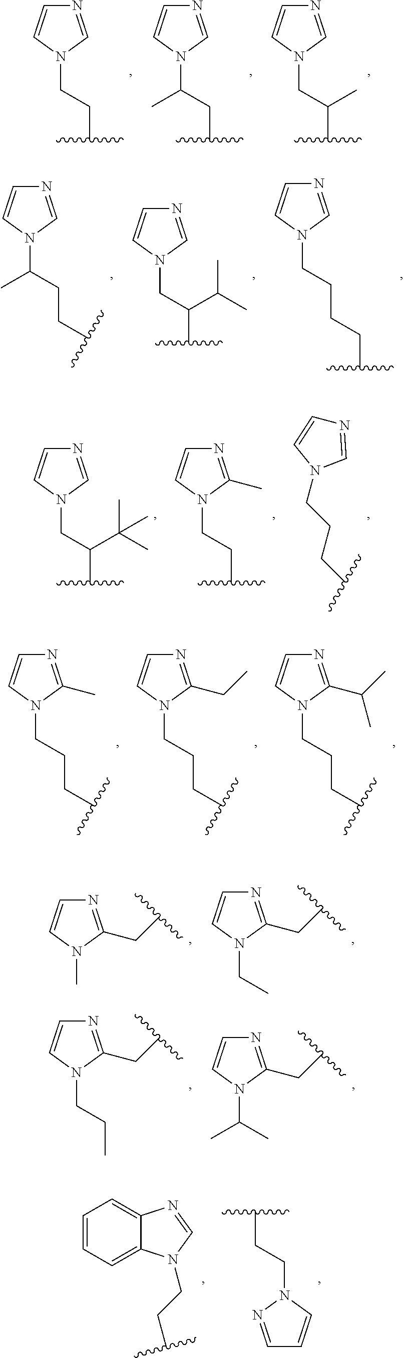

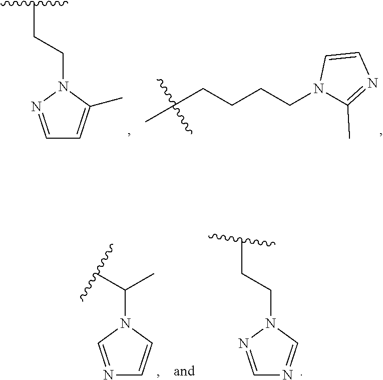

Embodiment 36. A pharmaceutical composition comprising:
(A) a circular RNA construct comprising:
   i. an IRES selected from an Enterovirus, Kobuvirus, Parechovirus, Hunnivirus, Passerivirus, Mischivirus, and Cardiovirus, and
   ii. at least one expression sequence encoding a CAR construct targeting a cancer antigen, and
(B) a transfer vehicle comprising:
(i) an ionizable lipid of Formula (I)

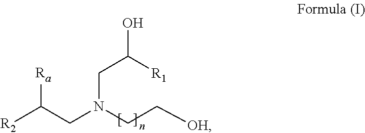

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
(ii) an ionizable lipid of Formula (II)

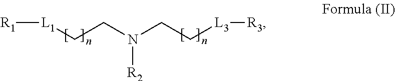

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

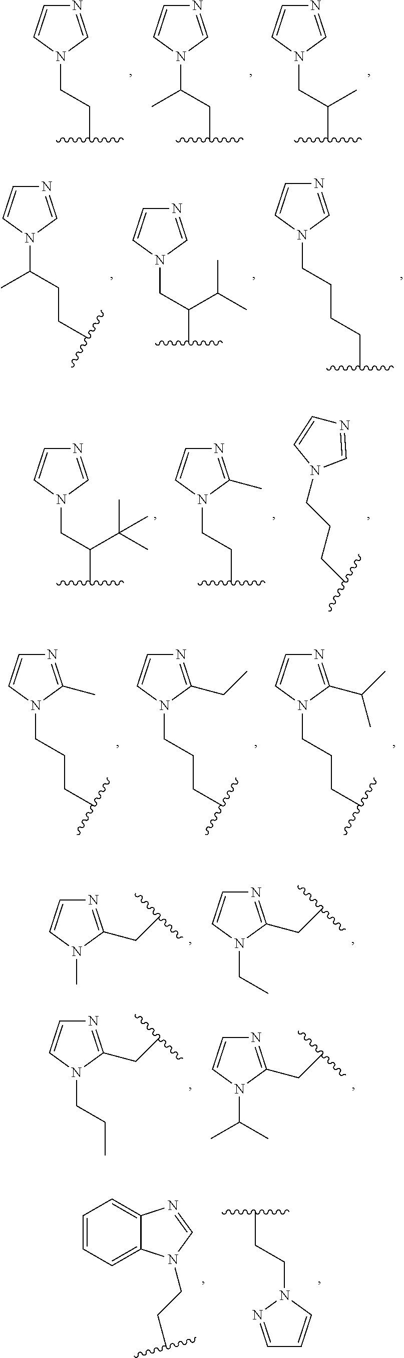

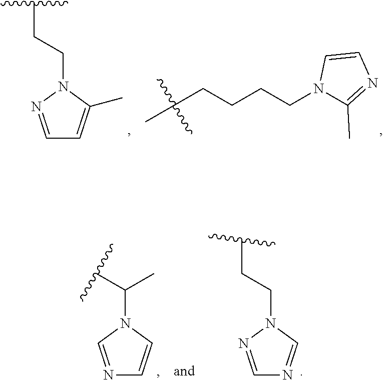

Embodiment 37. A pharmaceutical composition comprising:
(A) a circular RNA construct comprising:
   i. an IRES selected from an Enterovirus, Kobuvirus, Parechovirus, Hunnivirus, Passerivirus, Mischivirus, and Cardiovirus, and
   ii. at least one expression sequence encoding a CAR construct targeting a cancer antigen, wherein the CAR construct comprises a CD19 binder, and
(B) a transfer vehicle comprising:
(i) an ionizable lipid of Formula (I)

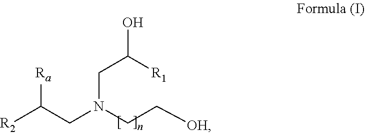

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
(ii) an ionizable lipid of Formula (II)

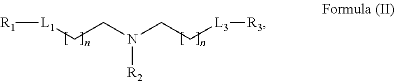

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

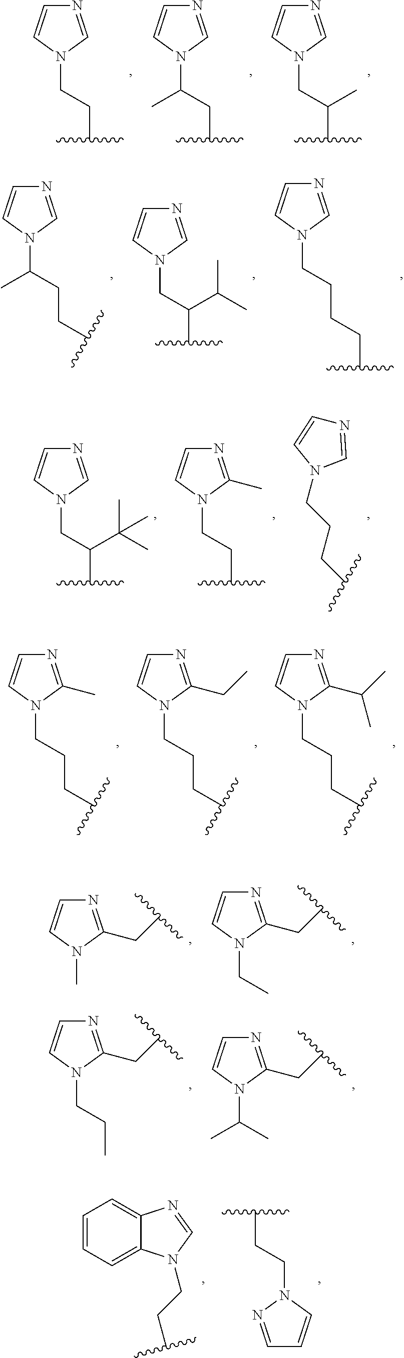

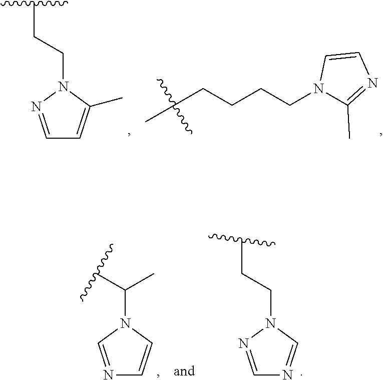

Embodiment 38. A pharmaceutical composition comprising:
(A) a circular RNA construct comprising:
   i. an IRES selected from an Enterovirus, Kobuvirus, Parechovirus, Hunnivirus, Passerivirus, Mischivirus, and Cardiovirus, and
   ii. at least one expression sequence encoding a CAR construct targeting a cancer antigen, wherein the CAR construct comprises an anti-CD19 binder, and wherein the expression sequence comprises a sequence that is at least 80% identical to a sequence selected from any one of SEQ ID NOs: 19-34, and
(B) a transfer vehicle comprising:
(i) an ionizable lipid of Formula (I)

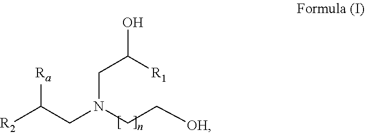

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
(ii) an ionizable lipid of Formula (II)

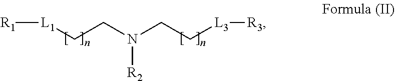

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

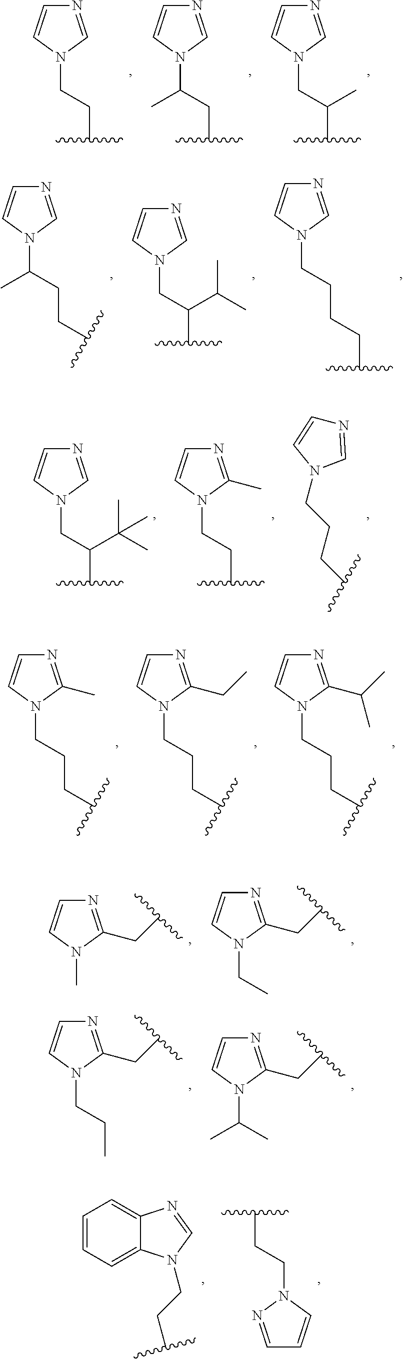

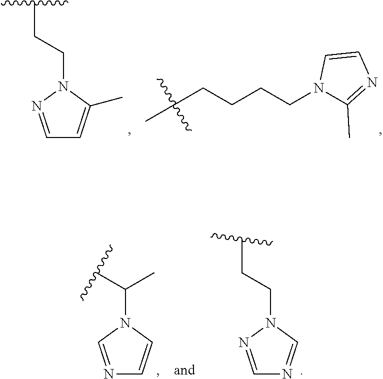

Embodiment 39. A pharmaceutical composition comprising:
(A) a circular RNA construct comprising:
   i. an IRES selected from an Enterovirus, Kobuvirus, Parechovirus, Hunnivirus, Passerivirus, Mischivirus, and Cardiovirus, and
   ii. at least one expression sequence encoding a CAR construct targeting a cancer antigen, wherein the CAR construct comprises a BCMA binder, and
(B) a transfer vehicle comprising:
(i) an ionizable lipid of Formula (I)

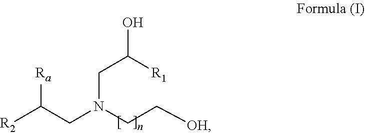

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
(ii) an ionizable lipid of Formula (II)

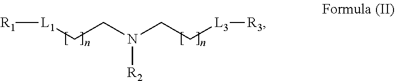

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

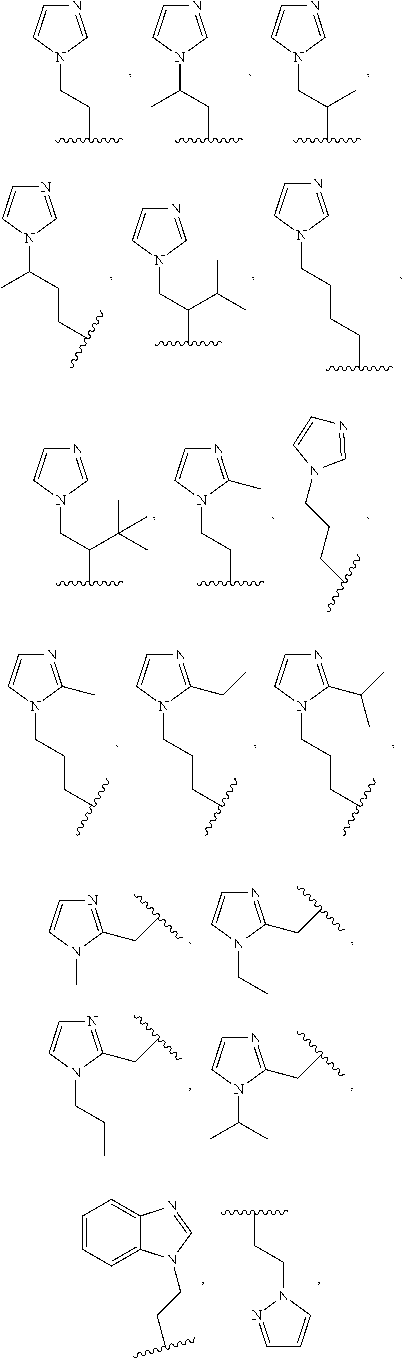

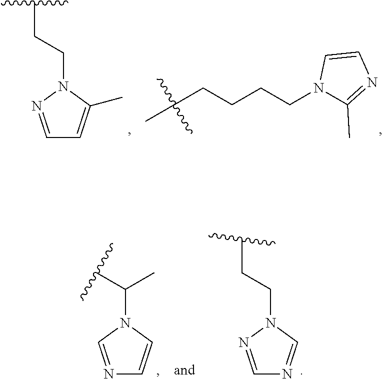

Embodiment 40. A pharmaceutical composition comprising:
(A) a circular RNA construct comprising:
   i. an IRES selected from an Enterovirus, Kobuvirus, Parechovirus, Hunnivirus, Passerivirus, Mischivirus, and Cardiovirus, and
   ii. at least one expression sequence encoding a CAR construct targeting a cancer antigen, wherein the CAR construct comprises an anti-BCMA binder, wherein the expression sequence comprises a sequence that is at least 80% identical to a sequence selected from any one of SEQ ID NOs: 103-115, and
(B) a transfer vehicle comprising:
(i) an ionizable lipid of Formula (I)

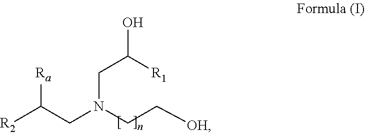

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
(ii) an ionizable lipid of Formula (II)

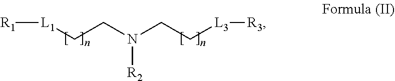

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidcalkyl, alkylsulfonyl, and alkylsulfonealkyl; and
R₂ is selected from a group consisting of:

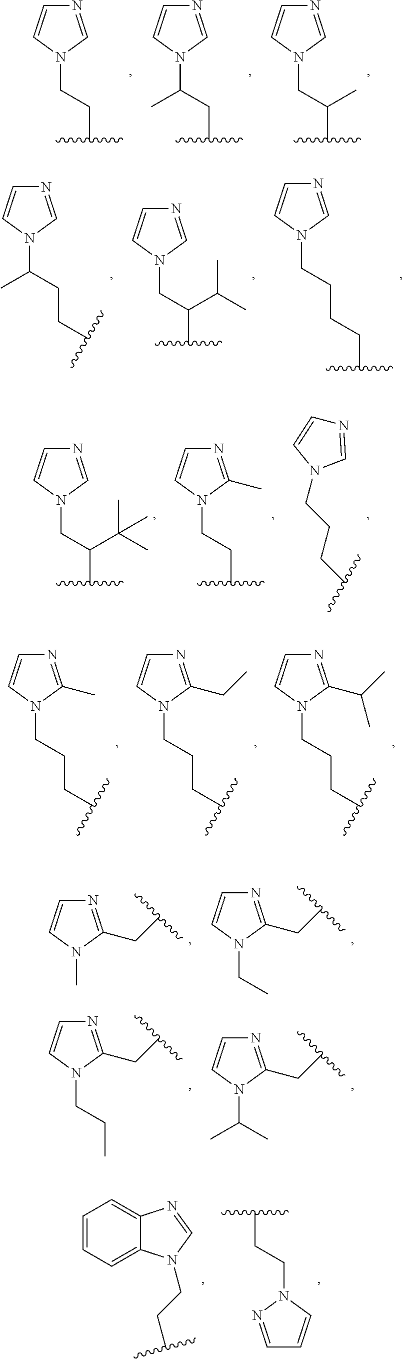

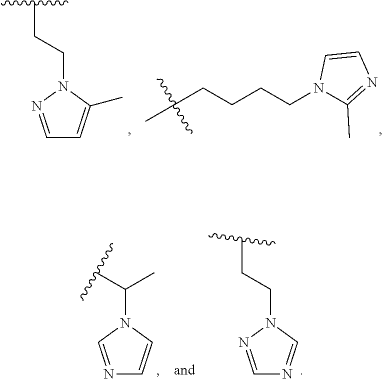

Embodiment 41. The pharmaceutical composition of any one of embodiments 1-4, 7-10, 13-20, 23-26, 29-32, and 35-38, wherein the CAR construct comprises a CD19 binder, and wherein the circular RNA comprises a sequence that is at least 80% identical to a sequence selected from any one of SEQ ID NOs: 50-61.

Embodiment 42. The pharmaceutical composition of any one of embodiments 1-4, 7-10, 13-20, 23-26, 29-32, and 35-38, wherein the CAR construct comprises a CD19 binder, and wherein the circular RNA comprises a sequence selected from any one of SEQ ID NOs: 50-61.

Embodiment 43. The pharmaceutical composition of embodiment 42, wherein the circular RNA construct comprises a sequence selected from any one of SEQ ID NOs: 50, 51, 52, 54, 55, 56, 58, and 59.

Embodiment 44. A pharmaceutical composition comprising a circular RNA construct and a transfer vehicle, wherein the circular RNA construct comprises an IRES and at least one expression sequence encoding a CAR construct targeting a cancer antigen, wherein the CAR construct comprises a CD19 binder, wherein the circular RNA comprises a sequence that is at least 80% identical to a sequence selected from any one of SEQ ID NOs: 50-61.

Embodiment 45. A pharmaceutical composition comprising a circular RNA construct and a transfer vehicle, wherein the circular RNA construct comprises an IRES and at least one expression sequence encoding a CAR construct targeting a cancer antigen, wherein the CAR construct comprises a CD19 binder, wherein the circular RNA construct comprises a sequence selected from any one of SEQ ID NOs: 50-61.

Embodiment 46. The pharmaceutical composition of embodiment 45, wherein the circular RNA construct comprises a sequence selected from any one of SEQ ID NOs: 50, 51, 52, 54, 55, 56, 58, and 59.

Embodiment 47. A pharmaceutical composition comprising a circular RNA construct and a transfer vehicle, wherein the circular RNA construct comprises an IRES and at least one expression sequence encoding a CAR construct targeting a cancer antigen, wherein the CAR construct comprises a CD19 binder, wherein the circular RNA comprises a sequence that is at least 80% identical to a sequence selected from any one of SEQ ID NOs: 50-61, and wherein the transfer vehicle comprises:
(i) an ionizable lipid of Formula (I)

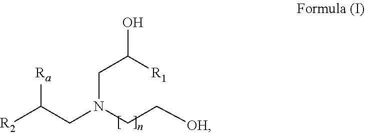

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
(ii) an ionizable lipid of Formula (II)

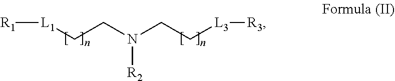

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

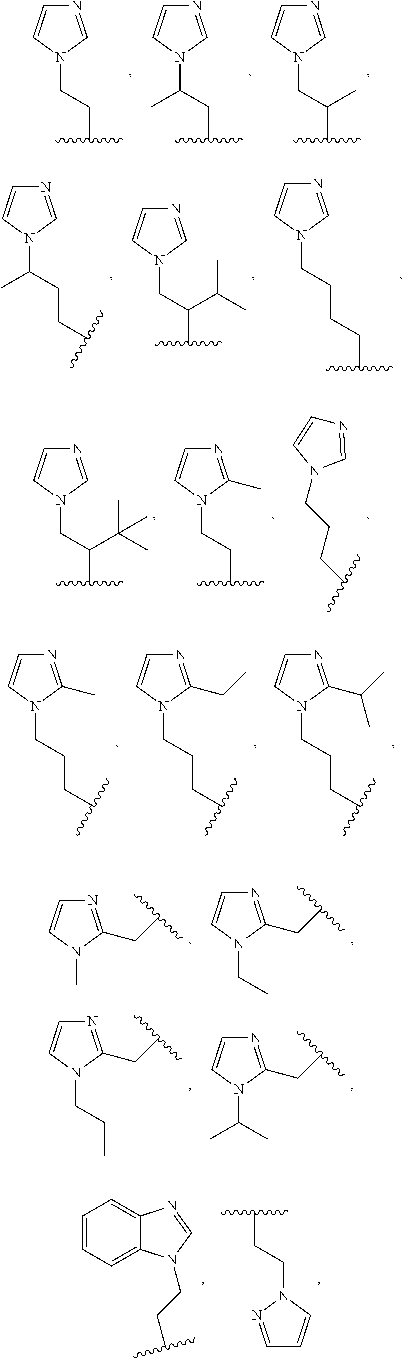

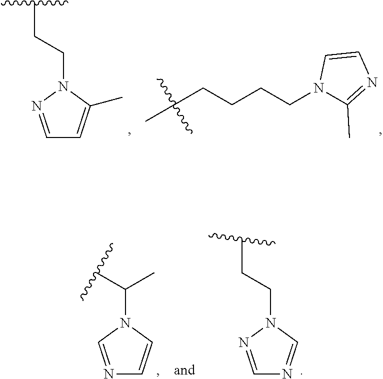

Embodiment 48. A pharmaceutical composition comprising a circular RNA construct and a transfer vehicle, wherein the circular RNA construct comprises an IRES and at least one expression sequence encoding a CAR construct targeting a cancer antigen, wherein the CAR construct comprises a CD19 binder, wherein the circular RNA comprises a sequence selected from any one of SEQ ID Nos: 50-61, and wherein the transfer vehicle comprises:
(i) an ionizable lipid of Formula (I)

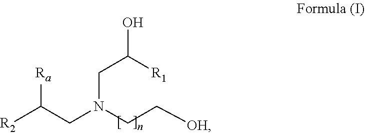

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
(ii) an ionizable lipid of Formula (II)

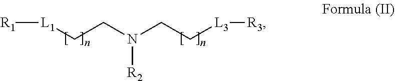

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

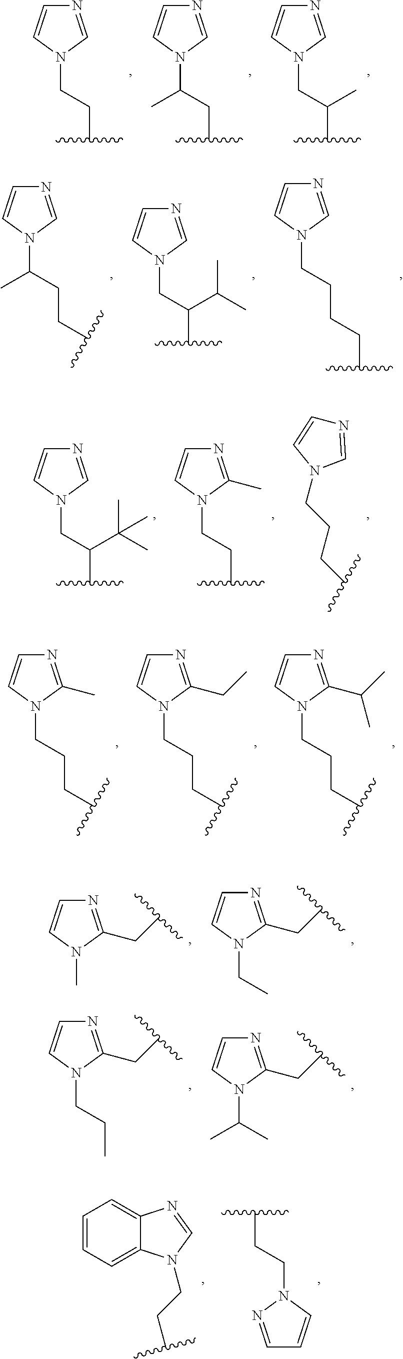

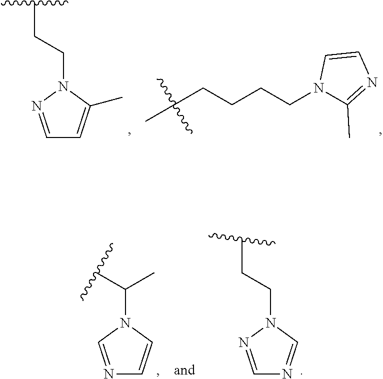

Embodiment 49. The pharmaceutical composition of embodiment 48, wherein the circular RNA construct comprises a sequence selected from any one of SEQ ID NOs: 50, 51, 52, 54, 55, 56, 58, and 59.

Embodiment 50. The circular RNA construct or pharmaceutical composition of any one of embodiments 1-49, wherein the circular RNA construct comprises SEQ ID NO: 50.

Embodiment 51. The circular RNA construct or pharmaceutical composition of any one of embodiments 1 to 49, wherein the circular RNA construct comprises SEQ ID NO: 51.

Embodiment 52. The circular RNA construct or pharmaceutical composition of any one of embodiments 1 to 49, wherein the circular RNA construct comprises SEQ ID NO: 52.

Embodiment 53. The circular RNA construct or pharmaceutical composition of any one of embodiments 1 to 49, wherein the circular RNA construct comprises SEQ ID NO: 54.

Embodiment 54. The circular RNA construct or pharmaceutical composition of any one of embodiments 1 to 49, wherein the circular RNA construct comprises SEQ ID NO: 55.

Embodiment 55. The circular RNA construct or pharmaceutical composition of any one of embodiments 1 to 49, wherein the circular RNA construct comprises SEQ ID NO: 56.

Embodiment 56. The circular RNA construct or pharmaceutical composition of any one of embodiments 1 to 49, wherein the circular RNA construct comprises SEQ ID NO: 58.

Embodiment 57. The circular RNA construct or pharmaceutical composition of any one of embodiments 1 to 49, wherein the circular RNA construct comprises SEQ ID NO: 59.

Embodiment 58. The pharmaceutical composition of any one of embodiments 5-6, 21-22, 27,28, 33-34, or 39-40, wherein the IRES comprises the sequence of SEQ ID NO: 8, wherein the CAR construct comprises a BCMA binder, and wherein the BCMA binder comprises a sequence selected from any one of SEQ ID NOs: 104-115.

Embodiment 59. The pharmaceutical composition of any one of embodiments 13-58, wherein the transfer vehicle comprises an ionizable lipid of Formula (I).

Embodiment 60. The pharmaceutical composition of embodiment 59, wherein the transfer vehicle comprises a helper lipid, a structural lipid, and a PEG-lipid.

Embodiment 61. The pharmaceutical composition of any one of embodiments 59-60, wherein the transfer vehicle has a lipid molar ratio formulation as described in Table 4b.

Embodiment 62. The pharmaceutical composition of any one of embodiments 13-58, wherein the transfer vehicle comprises an ionizable lipid of Formula (II).

Embodiment 63. The pharmaceutical composition of embodiment 62, wherein the ionizable lipid is selected from an ionizable lipid selected from:

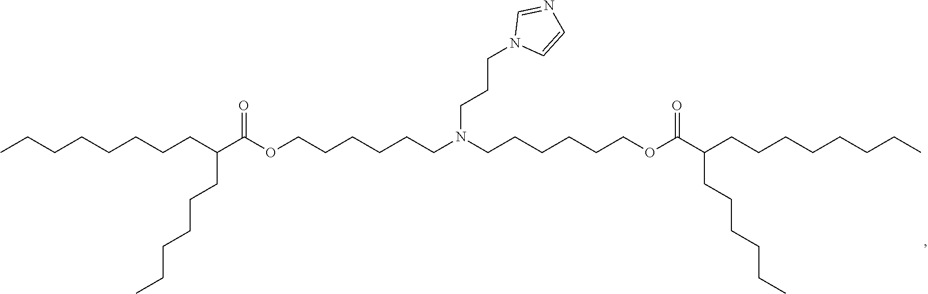 [SMILES: CCCCCCCCC(CCCCCC)C(=O)OCCCCCCN(CCCCCCOC(=O)C(CCCCCC)CCCCCCCC)CCCn1ccnc1]

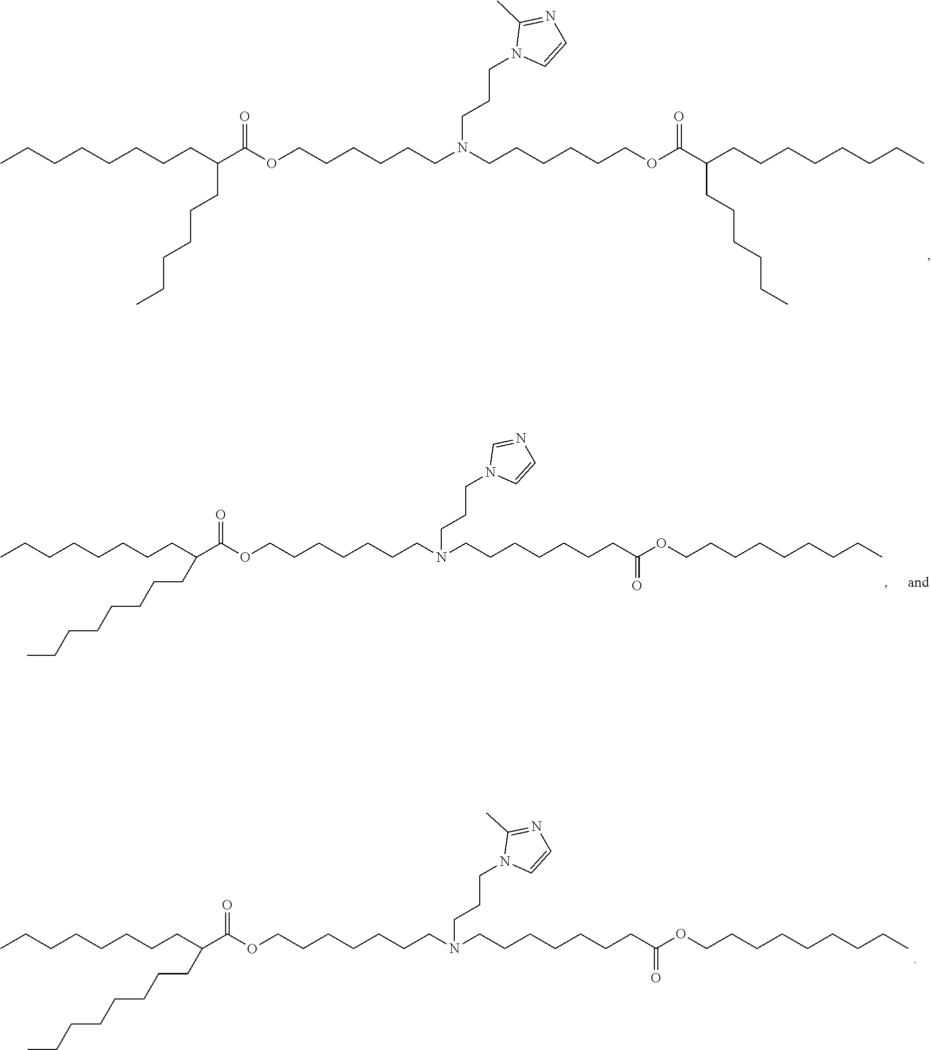

Embodiment 64. The pharmaceutical composition of embodiment 63, wherein the ionizable lipid is:

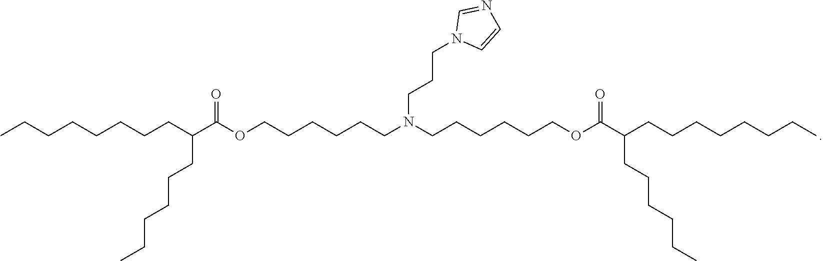 [SMILES: CCCCCCCCC(CCCCCC)C(=O)OCCCCCCN(CCCCCCOC(=O)C(CCCCCC)CCCCCCCC)CCCn1ccnc1]

Embodiment 65. The pharmaceutical composition of any one of embodiments 13-64, wherein the transfer vehicle further comprises at least one lipid selected from a helper lipid, a structural lipid, and a PEG-modified lipid.

Embodiment 66. The pharmaceutical composition of embodiment 65, wherein the transfer vehicle comprises PEG-DSPC.

Embodiment 67. The pharmaceutical composition of any one of embodiments 13-66, wherein the transfer vehicle is a lipid nanoparticle.

Embodiment 68. The pharmaceutical composition of any one of embodiments 13-67, wherein the transfer vehicle further comprises a targeting moiety.

Embodiment 69. The pharmaceutical composition of embodiment 68, wherein the targeting moiety is a small molecule, scFv, nanobody, peptide, cyclic peptide, di or tri cyclic peptide, minibody, polynucleotide aptamer, engineered scaffold protein, heavy chain variable region, light chain variable region, or a fragment thereof.

Embodiment 70. The pharmaceutical composition of any of embodiments 13-69, further comprising a pharmaceutical salt, buffer, diluent, or combination thereof.

Embodiment 71. The circular RNA construct or pharmaceutical composition of any one of the preceding embodiments, wherein the circular RNA further comprises a polyA region.

Embodiment 72. The circular RNA construct or pharmaceutical composition of any one of the preceding embodiments, wherein the circular RNA further comprises at least one miRNA binding site.

Embodiment 73. The circular RNA construct or pharmaceutical composition of embodiment 72, wherein the circular RNA comprises at least one miR-122 binding site.

Embodiment 74. The circular RNA construct or pharmaceutical composition of any one of the preceding embodiments, wherein the at least one expression sequence encoding a CAR is codon optimized.

Embodiment 75. The circular RNA construct or pharmaceutical composition of any one of the preceding embodiments, wherein the RNA construct further comprises a 5′ enhanced intron element, a 5′ enhanced exon element, a 3′ enhanced exon element, and a 3′ enhanced intron fragment.

Embodiment 76. A method of preparing the circular RNA construct or pharmaceutical composition of any one of the preceding embodiments.

Embodiment 77. A method of treating cancer in a subject by administering an effective amount of a composition comprising the circular RNA construct or the pharmaceutical composition of any one of embodiments 1-75, thereby treating the cancer. Additionally, a method of treating an autoimmune disease in a subject by administering an effective amount of a composition comprising the circular RNA construct or the pharmaceutical composition of any one of embodiments 1-75, thereby treating the autoimmune disease.

Embodiment 78. Use of a composition comprising the circular RNA construct or the pharmaceutical composition of any one of embodiments 1-75 for the treatment of cancer. Additionally, use of a composition comprising the circular RNA construct or the pharmaceutical composition of any one of embodiments 1-75 for the treatment of an autoimmune disease.

Embodiment 79. A linear precursor RNA polynucleotide comprising:
(A) an IRES comprising a sequence that is at least 80% identical to a sequence selected from any one of SEQ ID NOs: 1-18, and
(B) at least one expression sequence encoding a binding molecule.

Embodiment 80. A linear precursor RNA polynucleotide comprising:
(A) an IRES comprising a sequence that is at least 80% identical to a sequence selected from any one of SEQ ID NOs: 1-18, and
(B) at least one expression sequence encoding a CAR construct targeting a cancer antigen.

Embodiment 81. A linear precursor RNA polynucleotide comprising:
(A) an IRES comprising a sequence that is at least 80% identical to a sequence selected from any one of SEQ ID NOs: 1-18, and
(B) at least one expression sequence encoding a CAR construct targeting a cancer antigen, wherein the CAR construct comprises a CD19 binder.

Embodiment 82. The linear precursor RNA polynucleotide of embodiment 81, wherein the expression sequence comprises a sequence that is at least 80% identical to a sequence selected from any one of SEQ ID NOs: 19-34.

Embodiment 83. A linear precursor RNA polynucleotide comprising:
(A) an IRES comprising a sequence that is at least 80% identical to a sequence selected from any one of SEQ ID NOs: 1-18, and
(B) at least one expression sequence encoding a CAR construct targeting a cancer antigen, wherein the CAR construct comprises a BCMA binder.

Embodiment 84. The linear precursor RNA polynucleotide of embodiment 83, wherein the expression sequence comprises a sequence that is at least 80% identical to a sequence selected from any one of SEQ ID NOs: 103-115.

Embodiment 85. A linear precursor RNA polynucleotide comprising:
(A) an IRES selected from an Enterovirus, Kobuvirus, Parechovirus, Hunnivirus, Passerivirus, Mischivirus, and Cardiovirus, and
(B) at least one expression sequence encoding a binding molecule.

Embodiment 86. A linear precursor RNA polynucleotide comprising:
(A) an IRES selected from an Enterovirus, Kobuvirus, Parechovirus, Hunnivirus, Passerivirus, Mischivirus, and Cardiovirus, and
(B) at least one expression sequence encoding a CAR construct targeting a cancer antigen.

Embodiment 87. A linear precursor RNA polynucleotide comprising:
(A) an IRES selected from an Enterovirus, Kobuvirus, Parechovirus, Hunnivirus, Passerivirus, Mischivirus, and Cardiovirus, and
(B) at least one expression sequence encoding a CAR construct targeting a cancer antigen, wherein the CAR construct comprises a CD19 binder.

Embodiment 88. The linear precursor RNA polynucleotide of embodiment 87, wherein the expression sequence comprises a sequence that is at least 80% identical to a sequence selected from any one of SEQ ID NOs: 19-34.

Embodiment 89. A linear precursor RNA polynucleotide comprising:
(A) an IRES selected from an Enterovirus, Kobuvirus, Parechovirus, Hunnivirus, Passerivirus, Mischivirus, and Cardiovirus, and
(B) at least one expression sequence encoding a CAR construct targeting a cancer antigen, wherein the CAR construct comprises a BCMA binder.

Embodiment 90. The linear precursor RNA polynucleotide of embodiment 89, wherein the expression sequence comprises a sequence that is at least 80% identical to a sequence selected from any one of SEQ ID NOs: 103-115.

Embodiment 91. The linear precursor RNA polynucleotide of any one of embodiments 79 to 90, wherein the expression sequence is codon optimized.

Embodiment 92. The linear precursor RNA polynucleotide of any one of embodiments 79 to 91, further comprising a 5′ enhanced intron element, a 5′ enhanced exon element, a 3′ enhanced exon element, and a 3′ enhanced intron fragment.

Embodiment 93. The linear precursor RNA polynucleotide of embodiment 92 comprising the following order:
(A) the 5′ enhanced intron element,
(B) the 5′ enhanced exon element,
(C) a core functional element comprising the IRES and at least one expression sequence encoding a CAR construct targeting a cancer antigen, and optionally a stop codon or stop cassette,
(D) the 3′ enhanced exon element, and
(E) the 3′ enhanced intron element.

Embodiment 94. The linear precursor RNA polynucleotide of any one of embodiments 79 to 93, further comprising at least one miRNA binding site.

Embodiment 95. The linear precursor RNA polynucleotide of embodiment 94, wherein the precursor RNA comprises at least one miR-122 binding site.

Embodiment 96. A DNA vector encoding the RNA polynucleotide of any one of embodiments 79-95.

Embodiment 97. A method of preparing a circular RNA construct, the method comprising incubating the linear RNA polynucleotide of any one of embodiments 79-95 under suitable conditions for circularization.

Embodiment 98. A pharmaceutical composition comprising a circular RNA construct and a transfer vehicle, wherein the circular RNA construct comprises (i) an IRES comprising a sequence selected from any one of SEQ ID NOs: 1, 2, 4, and 8, and (ii) at least one expression sequence encoding a CAR construct targeting a cancer antigen, wherein the CAR construct comprises a CD19 binder comprising a sequence selected from any one of SEQ ID NOs: 19 and 20, and wherein the transfer vehicle is a lipid nanoparticle.

Embodiment 99. A pharmaceutical composition comprising a circular RNA construct and a transfer vehicle, wherein the circular RNA construct comprises (i) an IRES comprising a sequence selected from any one of SEQ ID NOs: 8, 16, 17, and 18, and (ii) at least one expression sequence encoding a CAR construct targeting a cancer antigen, wherein the CAR construct comprises a BCMA binder comprising SEQ ID NO: 115, and wherein the transfer vehicle is a lipid nanoparticle.

Embodiment 100. A pharmaceutical composition comprising a circular RNA construct and a transfer vehicle, wherein the circular RNA construct comprises (i) an IRES comprising a sequence selected from any one of SEQ ID NOs: 8, 16, 17, and 18, and (ii) at least one expression sequence encoding a CAR construct targeting a cancer antigen, wherein the CAR construct comprises a HER2 binder comprising a nucleotide sequences selected from any one of SEQ ID NO: 132 or 133, and wherein the transfer vehicle is a lipid nanoparticle.

Embodiment 101. The pharmaceutical composition of any one of embodiments 98 to 100, wherein the lipid nanoparticle comprises: (i) an ionizable lipid of Formula (I)

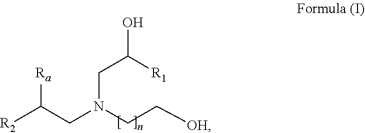

wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl;
or
   (ii) an ionizable lipid of Formula (II)

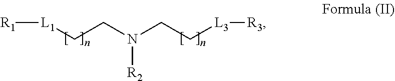

wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

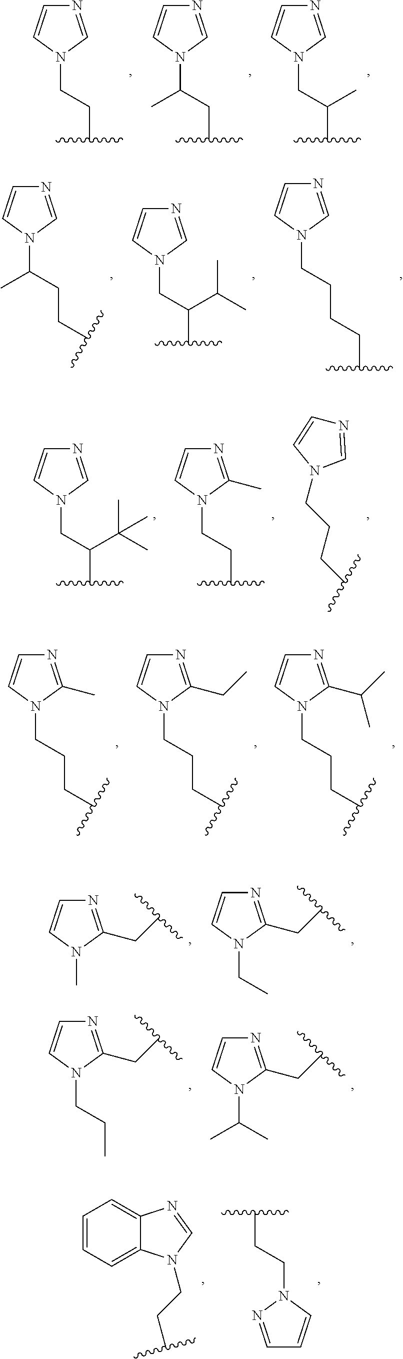

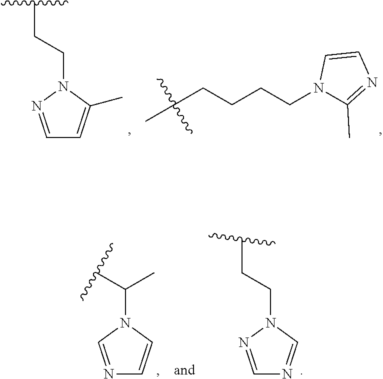

Embodiment 102. The pharmaceutical composition of embodiment 101, wherein the lipid nanoparticle transfer vehicle comprises an ionizable lipid, wherein the ionizable lipid is

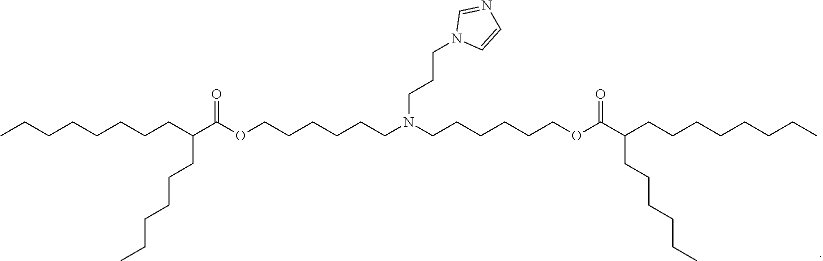 [SMILES: CCCCCCCCC(CCCCCC)C(=O)OCCCCCCN(CCCCCCOC(=O)C(CCCCCC)CCCCCCCC)CCCn1ccnc1]

Embodiment 103. The pharmaceutical composition of any one of embodiments 98-102, wherein lipid nanoparticle transfer vehicle further comprises at least one lipid selected from a helper lipid, a structural lipid, and a PEG-modified lipid.

Embodiment 104. A method of treating cancer comprising administering the pharmaceutical composition of any one of embodiments 98-103 to a human subject in need thereof.

Embodiment 105. A method of treating an autoimmune disease comprising administering the pharmaceutical composition of any one of embodiments 98-103 to a human subject in need thereof.

Embodiment 106. Use of a composition comprising the circular RNA construct for the treatment of cancer comprising administering the pharmaceutical composition of any one of embodiments 98-103 to a human subject in need thereof.

Embodiment 107. Use of a composition comprising the circular RNA construct for the treatment of an autoimmune disease comprising administering the pharmaceutical composition of any one of embodiments 98-103 to a human subject in need thereof.

Embodiment 108. The method of embodiment 77, or embodiment 104 or 105, or the use of embodiment 106 or 107, wherein the administering occurs every day, every other day, twice a week, every week, every ten days, every two weeks, every three weeks, every four weeks, once a month, every six weeks, every eight weeks, every three months, every four months, every six months, every eight months, every nine months, or annually.

### DESCRIPTION OF FIGURES

FIG. 1A shows a schematic of the sequence insertion site for exemplary IRES/codon plasmids. The IRES and the codon (expression sequence) were synthesized together and inserted into a circular RNA comprising a plasmid “backbone” containing bacterial sequences and 5′ combined accessory elements and 3′ combined accessory elements. The accessory elements can include, but are not limited to, the promoter, introns, exons, internal and external spacers, internal duplex regions, and polyA stretches. FIG. 1B depicts a general sequence construct of a linear RNA polynucleotide precursor (10). The sequence as provided is illustrated in a 5′ to 3′ order of a 5′ enhanced intron element (20), a 5′ enhanced exon element (30), a core functional element (40), a 3′ enhanced exon element (50) and a 3′ enhanced intron element (60). FIG. 1C shows an exemplary linear RNA polynucleotide precursor (10) comprising in the following 5′ to 3′ order, a leading untranslated sequence (21), a 5′ affinity tag (22), a 5′ external spacer (26), a 3′ intron fragment (28), a 3′ exon fragment (32), a 5′ internal duplex region (34), a 5′ internal spacer (36), a TIE (42), a coding element (46), a stop region (48), a 3′ internal spacer (52), a 3′ internal duplex region (54), a 5′ exon fragment (56), a 5′ intron fragment (62), a 3′ external spacer (64), a 3′ affinity tag (68), and a terminal untranslated sequence (69). FIG. 1D illustrates exemplary locations for an accessory element (70) (e.g., a miRNA binding site) included in a linear RNA polynucleotide located within the core functional element (40), for example where 42 is the TIE (translation initiation element), 46 is the coding region, 47 is the noncoding region, and 48 is the stop region (stop codon or stop cassette).

FIG. 2A and FIG. 2B depict a schematic of the preliminary process by which the combinations of IRES and codons were selected for the circRNA constructs.

FIG. 3A and FIG. 3B depict the effects of three different codon optimization algorithms. CD19 CAR+expression (gMFI) was evaluated via flow cytometry for each construct in two different donors (donor 4003 and donor 609C) and plotted in rank-order for all sequences and coded by codon optimization algorithm. White bars on the right indicate expression for the non-codon optimized CD19 CAR sequence (positive control).

FIGS. 4A, 4B, and 4C depict the effects of three different codon optimization algorithms. MFI (Total T cells), percent of CD3+ cells (CAR-T cell frequency), and Total cell count (CAR-T cell number) were evaluated over time post electroporation using the three algorithms as compared to a positive control and mock negative control.

FIGS. 5A, 5B, 5C, 5D, 5E, 5F, 5G, 5H, 5I, and 5J show T cell MFI (expression) by IRES for two donors (donor 4003 and donor 609C) over time (5 days) for circular RNA constructs comprising combinations of IRESes and expression sequences. Each point on the X axis is an IRES from Table 1A and each dot is a different expression sequence from Table 2A (codon optimized; anti-CD19 28-ζ).

FIGS. 6A, 6B, 6C, 6D, 6E, 6F, 6G, 6H, 6I, and 6J show % CAR positive cells by IRES over time, i.e., the percent of cells or average signal of the cells expressing over time (5 days) for two donors (donor 4003 and donor 609C) for circular RNA constructs comprising combinations of IRESes and expression sequences. Each point on the X axis is an IRES from Table 1A and each dot is a different expression sequence from Table 2A (codon optimized; anti-CD19 28-ζ).

FIG. 7A and FIG. 7B show the effect of varying the IRES on % Nalm6 Lysis data at 24 hours and 48 hours. Different constructs were created comprising the base CD19 codon (3276) were created in combination with different IRESes, including the IRES nos. 1-1, 1-4, 1-5, 1-6, 1-7, 1-8, 1-9, 1-10, 1-11, 1-12, 1-13, 1-14, and 1-15 in Table 1A (comprising SEQ ID NOs: 1, 4-15), as compared to a mock negative control, the IRES for a base CD19 CAR control, and Nalm6 alone in two different donors (609C and 4003).

FIG. 8A and FIG. 8B show cytotoxicity data in two different donors (609C and 4003) for the 69 CD19 CAR ORNA constructs identified by IRES/CO construct numbers in Table 5 as compared to a mock negative control, a base CD19 CAR control, and Nalm6 alone, ranked at 24 and 48 hours.

FIGS. 9A, 9B, 9C, and 9D reflect similar % Nalm6 killing cytotoxicity data for the CD19 CAR ORNA constructs as in FIGS. 8A and 8B for days 1 and 2 for the two donors, but is presented in a different visual format. Each point on the X axis is an IRES comprising the sequences of SEQ ID NOs: 1-15 (IRES nos. 1-1 to 1-15, described in Table 1A). Each dot is a different codon comprising the sequences of SEQ ID NOs 19-23 (codon nos. 2A-19 to 2A-23, described in Table 2A, codon optimized; anti-CD19 28-¿). The control is the IRES for a base CD19 CAR control (3276).

FIGS. 10A, 10B, 10C, and 10D reflect IFNγ expression and FIGS. 10E, 10F, 10G, and 10H reflect IL-2 expression at 24- and 48-hours post-electroporation for 69 CD19 CAR circular RNA constructs in two different donors (609C and 4003).

FIG. 11 shows Annexin V+Nalm6 (% of Nalm6) for a CD19 CAR construct of IRES/CO Clone #37 (SEQ ID NO: 52) as compared to a base CD19 CAR construct (3276) containing a non- optimized CAR sequence, a HER2 circular RNA construct (comprising HER2_9), a mock negative control, and Nalm6 alone.

FIG. 12A shows % of T cells over 96 hours for the 12 CD19 CAR constructs of Table 6 (identified by IRES/CO clone number) for Donor 9003. FIG. 12B shows CAR+MFI over 48 hours post electroporation for Donor 9003 for the 12 constructs. FIG. 12C shows Annexin V+Nalm6 over 72 hours as compared to a base CD19 CAR control (3276), a mock negative control, Nalm6 alone, and HER2.

FIG. 13A and FIG. 13B show IRES expression by luminescence for 12 different IRESes in 293 cells and Jurkat cell types.

FIG. 14 shows in vivo anti-tumor efficacy of CD19 oCAR constructs comprising IRES/CO clone numbers 7, 37, and 87 (SEQ ID NOs: 50, 52, 55, respectively) as compared to a base CD19 CAR control (3276), PBS, and a HER2 control, at dosages of 1.0 mg/kg, 0.3 mg/kg, and 0.1 mg/kg. Multiple Mann-Whitney test with Holm-Sidak correction, *p≤0.05, ** p≤0.01, *** p≤0.001.

FIG. 15 shows in vivo anti-tumor efficacy of CD19 oCAR constructs comprising IRES/CO clone numbers 97, 17, 164, and 87 (SEQ ID NOs: 56, 51, 58, 55, respectively) as compared to a base CD19 CAR control (3276), PBS, and a HER2 control, at dosages of 1.0 mg/kg, 0.3 mg/kg, and 0.1 mg/kg.

FIG. 16 shows total flux (photons/second) after 4 doses of LNP/oCAR for different lipid compositions comprising circular RNA constructs of HER2 and CD19 as compared to a control. The HER2 and CD19 lipid compositions comprise ionizable lipids 126, 128, 16, 45, and 86 of Table 3. Ionizable lipids 126 and 128 of Table 3 are lipids of Formula II and ionizable lipids 16, 45, and 86 of Table 3 are lipids of Formula I. A “/3” designation indicates the lipid composition comprises a PEG-modified lipid. Lipids (3-128)/3, (3-16)/3, (3-45)/3, and (3-86)/3 contain a PEG-modified lipid. Lipid (3-128)/3L contains ionizable lipid 128 of Table 3 and a PEG-modified lipid.

FIG. 17 shows % cytotoxicity (IncuCyte Cytotoxicity Assay) over time for the circular RNA constructs comprising HER2_9 and HER2_10 as compared to a base CD19 CAR control (3276) and a mock negative control.

FIGS. 18A, 18B, and 18C show % target lysis for a HER2.BBC oCAR construct and a HER2 285 oCAR construct as compared to a CD19 oCAR construct, a base CD19 CAR control, and mock negative control, evaluated using FACS based cytotoxicity assay after 24 hours co-culture using engineered HER2/K562 cell line (FIG. 18A), CD19/K562 (FIG. 18B), and Nalm6 (CD19+/HER2−) (FIG. 18C) cells.

FIGS. 19A, 19B, and 19C show target specific cytotoxicity for the oCAR construct comprising the sequence of BCMA_16 as compared to base CD19 CAR (3276) and a mock negative control in MM.1S cells, U266B1 cells, and Nalm 6 cells.

FIG. 20 shows tumor control as measured by total flux (photons/sec) post-Nalm6 engraftment using weekly dosing at 0.1 mg/kg (mpk) and 0.3 mg/kg (mpk).

FIGS. 21A, 21B, and 21C show tumor control as measured by total flux (photons/sec) post-Nalm6 engraftment using every-other-week (biweekly or q2w) dosing at 0.1 mg/kg and 0.3 mg/kg. Lipid 86 of Table 3 (“3-86”) was used for these oRNA CAR constructs.

FIG. 22 shows tumor control in vivo using every-other-week dosing at 0.1 mg/kg and 0.3 mg/kg.

FIG. 23A and FIG. 23B show quantitative tumor measurement over time post-Nalm6 engraftment. In FIG. 23A, the gray circles show response with control and the black squares show response with treatment with a CD19 oRNA CAR construct described herein. In FIG. 23B, the whole-body images show untreated and treated mice over time.

FIG. 24A depicts expression of BCMA CARs detected with soluble BCMA.PE post introduction of exemplary circular RNAs (circRNAs) via electroporation encoding BCMA-41BBζ CARs at 10 ng, 30 ng, or 100 ng dosages per 0.1×10⁶ T cell in comparison a “mock” control T cell not electroporated with any circRNAs. Expression was analyzed for the T cell at 24 hours, 48 hours, and 72 hours post introduction of the circular RNAs. FIG. 24B depicts expression of BCMA CARS quantified using geometric mean fluorescent intensity (gMFI) activity over the span of 24 hours post introduction of circular RNAs encoding BCMA-41BB (CARs at 10 ng, 30 ng, or 100 ng dosages per 0.1×10⁶ T cell. “A”, “B” and “C” correspond to “DNA Template A”, “DNA Template B”, and “DNA Template C” in Table al respectively, i.e., circular RNA construct “A” comprises the IRES sequence of DNA Template A and the BCMA sequence of DNA Template A; circular RNA construct B″ comprises the IRES sequence of DNA Template B and the BCMA sequence of DNA Template B; circular RNA construct “C” comprises the IRES sequence of DNA Template C and the BCMA sequence of DNA Template C; etc.

FIGS. 25A-25G depicts anti-BCMA chimeric antigen receptor (CAR) expression for exemplary circular RNAs encoding BCMA-41BBζ CAR post electroporation of the circular RNA into T cells. “A”, “B”, “C”, “D”, and “E” correspond to “DNA Template A”, “DNA Template B”, “DNA Template C”, “DNA Template D”, and “DNA Template E” in Table al respectively. “Mock” in the figure represents data for a control T cell that was not electroporated with circular RNA. FIG. 25A depicts percent CAR expression detected by soluble BCMA PE detection reagent over the span of 24-72 hours post electroporation of circRNAs formed from DNA Template A, DNA Template B, and DNA Template C and dosed at either 10 ng, 30 ng, or 100 ng per 0.1×10⁶ T cell into T cells. FIG. 25B depicts geometric mean fluorescence intensity (gMFI) of the T cells detected by soluble BCMA PE detection reagent over the span of 24-72 hours post electroporation of circRNAs formed from DNA Template A, DNA Template B, and DNA Template C and dosed at either 10 ng, 30 ng, or 100 ng per 0.1×10⁶ T cell into T cells. FIG. 25C provides fluorescence activated cell sorting (FACS) imaging post introduction of circular RNA depicted in FIGS. 25A and 25B to T cells at a dosage of 30 ng after 24 hours. FIG. 25D depicts percent CAR expression detected by soluble BCMA PE detection reagent over the span of 24-96 hours post electroporation of circRNAs formed from DNA Template A, DNA Template B, DNA Template C, DNA Template D, and DNA Template E and dosed at either 10 ng, 30 ng, or 100 ng per 0.1×10⁶ T cell into T cells. FIG. 25E depicts percent CAR expression detected by anti-Whitlow PE detection reagent over the span of 24-96 hours post electroporation of circRNAs formed from DNA Template A, DNA Template B, DNA Template C, DNA Template D, and DNA Template E and dosed at either 10 ng, 30 ng, or 100 ng per 0.1×10⁶ T cell into T cells. FIG. 25F depicts percent CAR expression detected by anti-G4S detection reagent over the span of 24-96 hours post electroporation of circRNAs formed from DNA Template A, DNA Template B, DNA Template C, DNA Template D, and DNA Template E and dosed at either 10 ng, 30 ng, or 100 ng per 0.1×10⁶ T cell into T cells. FIG. 25G depicts average MFI (%) of the T cells detected by soluble BCMA PE detection reagent over the span of 24-96 hours post electroporation of circRNAs formed from DNA Template A, DNA Template B, DNA Template C, DNA Template D, and DNA Template E and dosed at either 10 ng, 30 ng, or 100 ng per 0.1×10⁶ T cell into T cells.

FIG. 26 depicts an exemplary gating method used to analyze flow cytometry results for T cells electroporated with circular RNAs encoding BCMA CARs at a dose of 10 ng×10. BCMA CAR expression was detected with either soluble BCMA PE, anti-Whitlow PE or anti-G4S linker.

FIG. 27 shows target protein expression on multiple myeloma positive cells (e.g., MMIS, NCI-H929, and RPMI-8226) and negative target cell line (e.g., Nalm6 target cell line).

FIG. 28 depicts percent of live T cells collected at 24 hours post electroporation of circular RNAs comprising BCMA-41BB (CAR or CD19-CD285 CAR compared to “Mock” solutions comprising no circular RNA and only electroporation buffer solution. “F”, “C”, “G”, “H”, “A”, “I” and “J” correspond to “DNA Template F”, “DNA Template C”, “DNA Template G”, “DNA Template H”, “DNA Template A”, “DNA Template I”, and “DNA Template J” that were used to form the circular RNAs.

FIGS. 29A-29D provides gMFI collected from various circular RNA construct encoding a BCMA-41BBζ or BCMA-CD282 CAR or CD19-CD28° C. CAR electroporated onto T cells at a dosage of 50 ng per 0.1×10⁶ T cells compared to “Mock” control T cells lacking any circular RNA (containing only electroporation buffer). Each of the circular RNAs solutions were given either soluble BCMA (sBCMA-PE), anti-Whitlow-PE, or anti-G4S linker PE (G4S-AF647) detection reagent. FIG. 29A shows the histograms of the gMFI collected from the cells. FIGS. 29B-29D provides the gMFI for each of cells wherein sBCMA-PE (FIG. 29B), anti-Whitlow-PE (FIG. 29C) and G4S-AF647 (FIG. 29D) detection reagents were used to collect the gMFI. “F”, “C”, “G”, “H”, “A”, “T” and “J” correspond to “DNA Template F”, “DNA Template C”, “DNA Template G”, “DNA Template H”, “DNA Template A”, “DNA Template I”, and “DNA Template J” that were used to form the circular RNAs.

FIG. 30 depicts an exemplary gating process of the oCAR-T cells 24 hours post electroporation. On the top row of boxes (from left to right) provides the FACS imaging of lymphocytes, CD3 negative cells, live T cells, and BCMA positive cells. The bottom two boxes are histograms of BCMA CAR detected by either soluble BCMA or anti-Whitlow detection reagent (left bottom) or anti-GS4-PE Fluorescence (right bottom).

FIGS. 31A-31C depict percent expression of the detection reagent used (i.e., soluble BCMA PE (indicated by “sBCMA” in FIG. 31A), anti-Whitlow-PE (indicated by “Whitlow” in FIG. 31B) and anti-G4S linker PE (indicated by “G4S” in 31C)). Percent expression was calculated from the presence of the relevant detection reagent at 24 hours post electroporation of circular RNAs encoding BCMA-41BB (, BCMA-CD28ζ, or HER2 CAR gated on live T cells. “F”, “C”, “G”, “H”, “A”, “T” and “J” correspond to “DNA Template F”, “DNA Template C”, “DNA Template G”, “DNA Template H”, “DNA Template A”, “DNA Template I”, and “DNA Template J” that were used to form the circular RNAs. “Mock” in the figure represents data for a control T cell that was not electroporated with circular RNA.

FIGS. 32A-32E shows BCMA expression via gMFI (FIGS. 32A, 32B and 32D) or percent soluble BCMA PE detection (indicated as “% sBCMA-PE”) (FIG. 32C or 32E) post electroporation of circular RNAs encoding BCMA-41BBζ, BCMA-CD-CD285, or CD19-CD286 gated onto CD3+ cells. “Mock” indicates T cell solutions not electroporated with the circular RNA constructs. FIG. 32A provides a histogram with 24- and 48-hour collection of soluble BCMA-PE or anti-Whitlow.PE detection for the circular RNA constructs. FIGS. 32B and 32C provide gMFI and % sBCMA-PE expression over the span of 24-72 for each of the constructs after CD3+ cells comprising the circular RNAs have been co-cultured with multiple myeloma (MMIS) cells. FIGS. 32D and 32E provide gMFI and % sBCMA-PE expression at 72 post electroporation for each of the constructs after CD3+ cells comprising the circular RNAs have been co-cultured with multiple myeloma (MMIS) cells, NCI-H929 (indicated in the figures as “H929”), Nalm6 or K562.CD19 cells. “C”, “G”, “H”, “A”, “I” and “J” correspond to “DNA Template C”, “DNA Template G”, “DNA Template H”, “DNA Template A”, “DNA Template I”, and “DNA Template J” that were used to form the circular RNAs. “Mock” in the figure represents data for a control T cell that was not electroporated with circular RNA.

FIGS. 33A-33C depict cytotoxicity of circular RNA constructs encoding a BCMA-41BBC chimeric antigen receptor (CAR), wherein the circular RNA comprises a BCMA sequence and IRES sequence from Table al, B or y CD19-CD28ζ CAR or HER2-CD28° C. CARs on various cell types over the span of 0 to 72 or 96 hours post co-culture. FIG. 33A provides cytotoxicity of each of the circular RNAs encoding CAR constructs at a dosage of either 10 or 30 ng per 0.1×10⁶ T cells on MMIS cells. Mock T cells (i.e., T cells not electroporated circular RNA referred to as “Mock” in the figure) and MMIS cells not co-cultured with T cells indicated as “MMIS” in FIG. 33A were used as controls. FIG. 33B provides cytotoxicity of each of the circular RNAs encoding CAR constructs at a dosage of either 10 or 30 ng per 0.1×10⁶ T cells on Nalm6 cells. Mock T cells (i.e., T cells not electroporated circular RNA indicated as “Mock” in the figure) and Nalm6 cells not co-cultured with T cells as referred to as in FIG. 33B as “Nalm6” were used as controls. FIG. 33C provides cytotoxicity of each of the circular RNAs encoding CAR constructs at a dosage of 20 ng per 0.1×10⁶ T cells on CD19 T stable cell line. Mock T cells (i.e., tumor T cells not electroporated circular RNA indicated as “Mock” in the figure) and CD19 T stable cells not co-cultured with T cells referred to as in FIG. 33C as “tumor” were used as controls. % Cytotoxicity was calculated by the (green area+red area/green area) produced by the live-cell analysis portfolio system imaging. “A”, “B”, “C”, “F”, and “K” correspond to “DNA Template A”, “DNA Template B”, “DNA Template C”, “DNA Template F”, and “DNA Template K” that were used to form the circular RNAs.

FIGS. 34A-34C depict cytotoxicity analysis of various engineered circular RNAs across multiple cell types. FIG. 34A provides the FACS imaging of the cells (e.g., lymphocytes, CD3 negative cells, live cells, and BCMA positive cells) at 24 hours post co-culture of oCAR-T cells formed from introduction of circular RNA comprising a 3′ Anabeana exon, a Caprine Kobuvirus internal ribosome entry site (IRES), a BCMA-41BBζ CAR, and a 5′ Anabaena exon. FIG. 34B shows the percent cytotoxicity acquired from circular RNAs encoding BCMA-41BBζ, CD19-CD28° C. or HER2-CD28° C. CARs on MMIS (FIG. 24B) or Nalm6 (FIG. 24C). “MMIS+Mock” and “MMIS” as depicted in FIG. 34B refers to MMIS cell that was co-cultured with a T cell that was not transfected with a circular RNA. “Nalm6+Mock” and “Nalm6” as depicted in FIG. 34C refers to a Nalm6 cell that was co-cultured with a T cell that was not transfected with a circular RNA. “A”, “B”, “C”, “F”, and “K” correspond to “DNA Template A”, “DNA Template B”, “DNA Template C”, “DNA Template F”, and “DNA Template K” that were used to form the circular RNAs.

FIG. 35A depicts FACS imaging of “Mock+MMIS” (i.e., MMIS tumor cells cocultured with T cells not electroporated with circular RNAs, “Mock+Nalm6” (i.e., Nalm6 tumor cells cocultured with T cells not electroporated with circular RNAs), “Mock+H929” (i.e., NCI-H929 tumor cells cocultured with T cells not electroporated with circular RNAs), and “Mock+K562.CD19” (i.e., K562.CD19 tumor cells cocultured with T cells not electroporated with circular RNAs). FIG. 35B depicts FACS imaging for CD19+CD3+ cells.

FIG. 36A-36D depicts % target cell viability (top) and % target cell killing of T cells (bottom) that have been electroporated with circular RNAs derived from DNA Templates in Tables al, β and/or γ1 and later co-cultured with a target cell (e.g., MMIS (FIG. 36A), NCI-H929 (depicted as “H929” in FIG. 36B), Nalm6 (FIG. 36C), or K562.CD19 (FIG. 36D)) for 24 (left) or 48 (right) hours post co-culture. “Mock+MMIS” (i.e., MMIS tumor cells cocultured with T cells not electroporated with circular RNAs, “Mock+Nalm6” (i.e., Nalm6 tumor cells cocultured with T cells not electroporated with circular RNAs), “Mock+H929” (i.e., NCI-H929 tumor cells cocultured with T cells not electroporated with circular RNAs), and “Mock+K562.CD19” (i.e., K562.CD19 tumor cells cocultured with T cells not electroporated with circular RNAs). “A”, “G”, “C”, “F”, “H”, “I”, and “J” correspond to “DNA Template A”, “DNA Template G”, “DNA Template C”, “DNA Template F”, “DNA Template H”, “DNA Template I”, and “DNA Template J” that were used to form the circular RNAs.

FIGS. 37A and 37B depict INγ cytokine secretion produced from circular RNAs encoding BCMA-41BBζ, CD19-CD28ζ and HER2-CD286 CARs at 10, 30, or 100 ng dose per 0.1×10⁶ T cells on MMIS (FIG. 37A) or Nalm6 (FIG. 37B) cells post co-culture of the MMIS or Nalm6 with T cells containing the circular RNAs. Cytotoxicity levels were calculated from a cytokine and chemokine kit (e.g., MSD). “MMIS+Mock” refers to MMIS tumor cells that were co-cultured with T cells that had not be electroporated with circular RNAs. “MMIS” refers to tumor cells that were not co-cultured with T cells. “Nalm6+Mock” refers to Nalm6 tumor cells that were co-cultured with T cells that had not been electroporated with circular RNAs. “Nalm6” refers to tumor cells that were not co-cultured with T cells. “A”, “B”, “C”, “F”, and “K” correspond to “DNA Template A”, “DNA Template B”, “DNA Template C”, “DNA Template F”, and “DNA Template K” that were used to form the circular RNAs.

FIGS. 38A-38P depict cytokine levels (pg/mL) at 24 and 48 hours (left and right respectively in each figure) in cocultured T cells comprising circular RNA encoding BMCA-41BBζ, BCMA-CD28ζ, or CD19-CD28ζ CAR and target cells. The target cells include MMIS (FIGS. 38A, 38E, 38I and 38M), NCI-H929 (indicated as “H929”) (FIGS. 38B, 38F, 38J, 38N), Nalm6 (FIGS. 38C, 38G, 38K, 380), and K562.CD19 (FIGS. 38D, 38H, 38L, 38P). FIGS. 38A-38D provide INFγ cytokine levels, FIGS. 38E-38H provides TNFα cytokine levels, FIGS. 381-38L provides IL-2 cytokine levels, and FIGS. 38M-38P GM-CSF levels. “A”, “G”, “C”, “F”, “H”, “T”, and “J” correspond to “DNA Template A”, “DNA Template G”, “DNA Template C”, “DNA Template F”, “DNA Template H”, “DNA Template I”, and “DNA Template J” that were used to form the circular RNAs.

FIG. 39 depicts percent apoptosis of target cell (e.g., Nalm6) collected from live-cell analysis portfolio system (e.g., an IncuCyte) (e.g., % apoptotic target cells=(green area+red area)/green area) over the span of 72 hours post introduction of circular RNAs encoding HER2 CAR. Green areas indicate target cells. Red areas indicate Annexin V reagent present in the apoptotic cells. For control, Nalm6 comprising no circular RNAs was used.

FIGS. 40A-40C depicts % Annexin V/phase post introduction of circular RNAs encoding HER2.28ζ, HER2. BBC or CD19.28ζ CAR to activated PBMC T cells and co-cultured in BT474 (FIG. 40A), SKBR3 (FIG. 40B), and JIMT1 (FIG. 40C) HER2 positive cell. For comparison purposes, activated PBMC T cells lacking any circular RNAs were used (indicated as “Mock”). “% Annexin V/phase” as referenced in FIGS. 40A-40C pertains to percent of apoptotic cells per phase. “K”, “L”, and “M” correspond to “DNA Template K”, “DNA Template L”, and “DNA Template M” that were used to form the circular RNAs.

FIG. 41A depicts frozen and fresh LNP delivered CAR expression of three different circular RNA constructs encoding HER2 CAR. “Mock” cells were T cells given empty LNPs (without circular RNAs). FIG. 41B provides % cytotoxicity collected from live-cell analysis portfolio system (e.g., an IncuCyte) analysis of T cells comprising circular RNA constructs encoding HER2-28ζ, HER2-BBζ or CD19-28ζ CAR cocultured with BT-474 target cells at a 1:1 ET ratio, wherein the circular RNAs were delivered with either a fresh or frozen LNP. The fresh and frozen LNPs comprised an ionizable lipid from Table 3. FIG. 41C provides the cytokine release (top graph of FIG. 41C: INFγ and bottom graph of FIG. 41C: TNFα) produced by the T cells co-cultured in the BT-474 for each of the circular RNA constructs. “K”, “L”, and “F” correspond to “DNA Template K”, “DNA Template L”, and “DNA Template F” that were used to form the circular RNAs. “Fresh” indicates that the LNP was not previously frozen. “Frozen” indicates that the LNP was previously frozen.

FIGS. 42A-42L depict anti-HER2 expression of circular RNAs encoding HER2.286, HER2.BBC or CD19.28ζ CAR injected intravenously and delivered using lipid nanoparticles into JIMT-1 (FIGS. 42A-42L) and BT-474 (FIGS. 42G-42L) mouse models. FIGS. 42C-42F are some of the spider plots of the data collected in FIGS. 42A-42B. FIGS. 421-42L are spider plots of the data collected in FIGS. 42G and 42H. “K”, “L”, and “F” correspond to “DNA Template K”, “DNA Template L”, and “DNA Template F” that were used to form the circular RNAs. “Fresh” indicates that the LNP was not previously frozen. “Frozen” indicates that the LNP was previously frozen.

FIGS. 43A-D show CAR expression after electroporation in circular RNA constructs comprising an IRES sequence, an anti-HER2 CAR, and a 28z domain (HER2_9, HER2_1, HER2_3, HER2 4, described herein in Table 9).

FIGS. 44A-D show CAR expression after electroporation in circular RNA constructs comprising an IRES sequence, an anti-HER2 CAR, and a BBz domain (HER2_10, HER2_5, HER2_7, HER2_8, described herein in Table 9).

FIGS. 45A and 45B show performance of circular RNA constructs comprising an IRES sequence, an anti-HER2 CAR, and a 28z or BBz domain (HER2_10, HER2_5, HER2_7, HER2_8), as compared to control (3273 (base) and mock), in BT474 target cells.

FIG. 46 shows an exemplary method for assessing the ability of a circular RNA comprising anti-CD19 CAR (in situ CAR or isCAR™) to deplete human B cells in a CD34+engrafted humanize mouse model.

FIG. 47 shows an exemplary flow cytometry panel used in an autoimmunity study.

FIGS. 48A-C show B cell depletion mediated by a circular RNA comprising anti-CD19 CAR.

FIGS. 49A-C show splenic B cells were depleted in mice treated with a circular RNA encoding a reporter (m Wasabi) encapsulated in a lipid nanoparticle as described herein.

FIG. 50 shows an exemplary method for assessing RAJI control in NK cells using circular RNA comprising anti-CD19 CAR in NOG-IL15 mice.

FIG. 51 shows NOG-IL15 mice engrafted with CD19+Raji-luc cell line at Day 0. On Day 3, primary human NK cells were purified from peripheral blood and were engrafted into recipient animals. On day 8, mice were left untreated, or treated with i.v. with vehicle, LNP-1 mg/kg mOX40L CAR or 1 mg/kg LNP-CD19 CAR. Mice were treated every two days for 10 doses. Tumor burden was imaged using IVIS imaging. Data show that mice treated with LNP-CD19 CAR show tumor control until day 24, study endpoint.

FIG. 52 shows an exemplary method for assessing circular RNA in macrophages.

FIG. 53 shows an exemplary FACS gating strategy for establishing circular RNA delivery to monocytes as applied elsewhere herein.

FIGS. 54A-D show mOX40L expression in myeloid cells in bone marrow.

FIGS. 55A-G show mOX40L expression in CD33+CD14+ and CD14-cells in bone marrow.

FIGS. 56A-G show mOX40L expression in CD33+CD64+ and CD64-cells in bone marrow.

FIGS. 57A-D show mOX40L expression in myeloid cells in spleen.

FIGS. 58A-G show mOX40L expression in CD33+CD14+ and CD14-cells in spleen.

FIGS. 59A-G show mOX40L expression in CD33+CD64+ and CD64-cells in spleen.

FIGS. 60A and B show tumor control in vivo by circular RNA encoding BCMA CAR (BCMA oCAR, BCMA_7 and BCMA_3) when dosed EOD in two donors as compared to HER2 and no-treatment controls.

FIGS. 61A and B show tumor control in vivo by circular RNA encoding BCMA CAR (BCMA oCAR, BCMA_7) when dosed once weekly (QW) in multiple donors as compared to HER2 and no-treatment controls.

FIG. 62 shows tumor control in vivo by circular RNA encoding BCMA CAR (BCMA oCAR, BCMA_7 and BCMA_3) in exemplary IVIS images when dosed every other day (EOD) and once weekly (QW) as compared to HER2 and no-treatment controls.

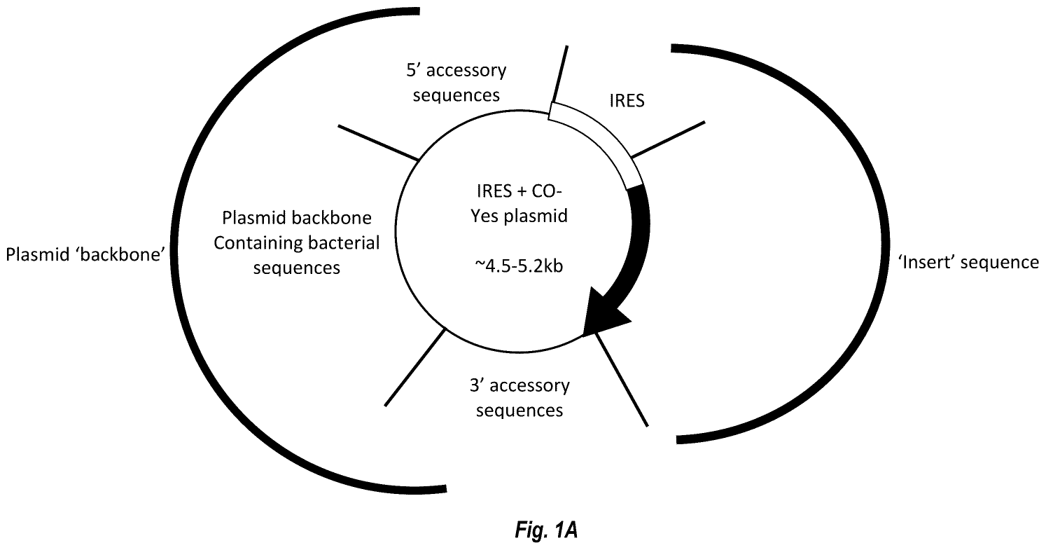

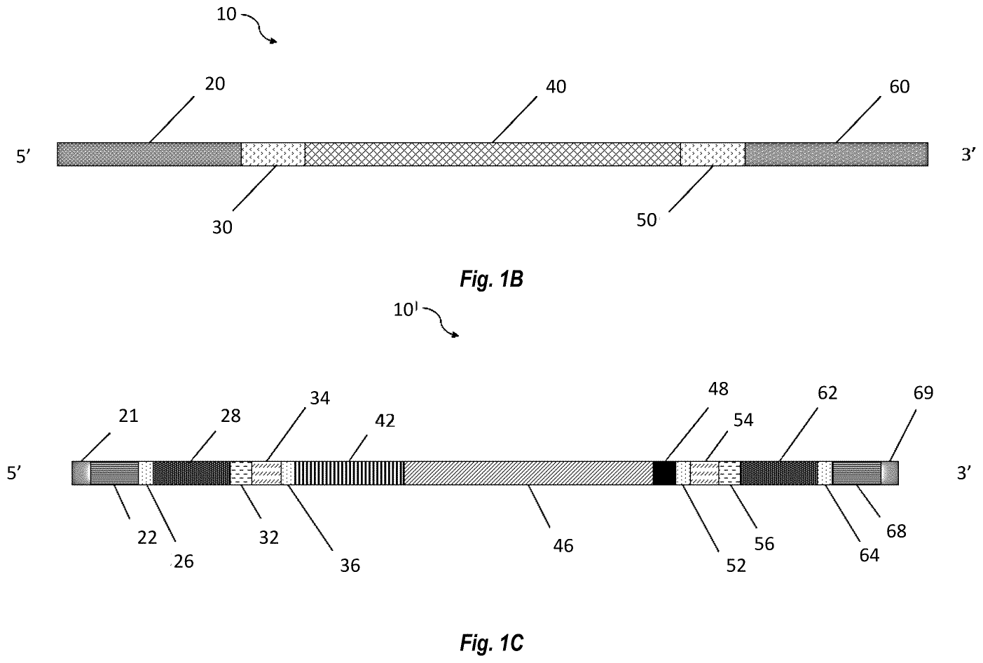

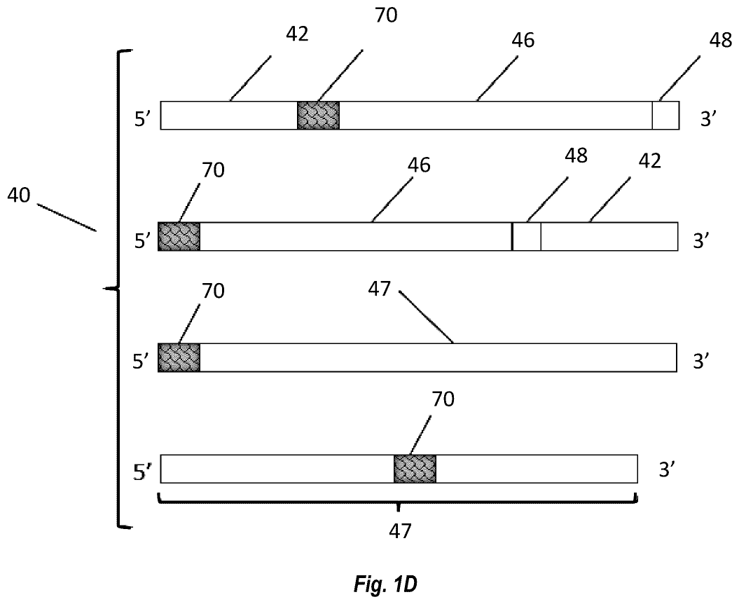

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a guide” includes a plurality of guides and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

As used herein, the term “circRNA,” “circular polyribonucleotide,” “circular RNA,” “circularized RNA,” or “ORNA” are used interchangeably and refer to a single-stranded RNA polynucleotide wherein the 3′ and 5′ ends that are normally present in a linear RNA polynucleotide have been joined together.

As used herein, the term “DNA template” refers to a DNA sequence capable of transcribing a linear RNA polynucleotide. For example, but not intending to be limiting, a DNA template may include a DNA vector, PCR product or plasmid.

As used herein, the term “3′ group I intron fragment” refers to a sequence with 75% or higher similarity to the 3′-proximal end of a natural group I intron including the splice site dinucleotide and optionally a stretch of natural exon sequence. In some embodiments, a circular RNA comprises a post splicing 3′ group I intron fragment. In some embodiments, the post splicing 3′ group I intron fragment in the circular RNA is a post splicing stretch of exon sequence. In some embodiments, the circular RNA further comprises a desired expression sequence, and the post splicing stretch of exon sequence is (e.g., designed) to be a portion of the desired expression sequence, contiguous with the desired expression sequence, and/or in frame with the desired expression sequence.

As used herein, the term “5′ group I intron fragment” refers to a sequence with 75% or higher similarity to the 5′-proximal end of a natural group I intron including the splice site dinucleotide and optionally a stretch of natural exon sequence. In some embodiments, a circular RNA comprises a post splicing 5′ group I intron fragment. In some embodiments, the post splicing 5′ group I intron fragment in the circular RNA is a post splicing stretch of exon sequence. In some embodiments, the circular RNA further comprises a desired expression sequence, and the post splicing stretch of exon sequence is (e.g., designed) to be a portion of the desired expression sequence, contiguous with the desired expression sequence, and/or in frame with the desired expression sequence.

As used herein, the term “permutation site” refers to the site in a group I intron where a cut is made prior to permutation of the intron. This cut generates 3′ and 5′ group I intron fragments that are permuted to be on either side of a stretch of precursor RNA to be circularized.

As used herein, the term “splice site” refers to a dinucleotide that is partially or fully included in a group I intron and between which a phosphodiester bond is cleaved during RNA circularization. (As used herein, “splice site” refers to the dinucleotide or dinucleotides between which cleavage of the phosphodiester bond occurs during a splicing reaction. A “5′ splice site” refers to the natural 5′ dinucleotide of the intron e.g., group I intron, while a “3′ splice site” refers to the natural 3′ dinucleotide of the intron).

As used herein, the term “expression sequence” refers to a nucleic acid sequence that encodes a product, e.g., a peptide or polypeptide, regulatory nucleic acid, or non-coding nucleic acid. An exemplary expression sequence that codes for a peptide or polypeptide can comprise a plurality of nucleotide triads, each of which can code for an amino acid and is termed as a “codon.”

As used herein, “coding element” or “coding region” is region located within the expression sequence and encodings for one or more proteins or polypeptides (e.g., therapeutic protein).

As used herein, a “noncoding element,” “noncoding region,” or “non-coding nucleic acid” is a region located within the expression sequence. This sequence by itself does not encode for a protein or polypeptide, but may have other regulatory functions, including but not limited to allowing the overall polynucleotide to act as a biomarker or adjuvant to a specific cell.

As used herein, the term “therapeutic protein” refers to any protein that, when administered to a subject directly or indirectly in the form of a translated nucleic acid, has a therapeutic, diagnostic, and/or prophylactic effect and/or elicits a desired biological and/or pharmacological effect.

As used herein, the term “immunogenic” refers to a potential to induce an immune response to a substance. An immune response may be induced when an immune system of an organism or a certain type of immune cells is exposed to an immunogenic substance. The term “non-immunogenic” refers to a lack of or absence of an immune response above a detectable threshold to a substance. No immune response is detected when an immune system of an organism or a certain type of immune cells is exposed to a non-immunogenic substance. In some embodiments, a non-immunogenic circular polyribonucleotide as provided herein, does not induce an immune response above a pre-determined threshold when measured by an immunogenicity assay. In some embodiments, no innate immune response is detected when an immune system of an organism or a certain type of immune cells is exposed to a non-immunogenic circular polyribonucleotide as provided herein. In some embodiments, no adaptive immune response is detected when an immune system of an organism or a certain type of immune cell is exposed to a non-immunogenic circular polyribonucleotide as provided herein.

As used herein, the term “translation efficiency” refers to a rate or amount of protein or peptide production from a ribonucleotide transcript. In some embodiments, translation efficiency can be expressed as amount of protein or peptide produced per given amount of transcript that codes for the protein or peptide.

The term “nucleotide” refers to a ribonucleotide, a deoxyribonucleotide, a modified form thereof, or an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Nucleotide analogs include nucleotides having modifications in the chemical structure of the base, sugar and/or phosphate, including, but not limited to, 5′-position pyrimidine modifications, 8′-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH2, NHR, NR2, or CN, wherein R is an alkyl moiety as defined herein. Nucleotide analogs are also meant to include nucleotides with bases such as inosine, queuosine, xanthine; sugars such as 2′-methyl ribose; non-natural phosphodiester linkages such as methylphosphonate, phosphorothioate and peptide linkages. Nucleotide analogs include 5-methoxyuridine, 1-methylpseudouridine, and 6-methyladenosine.

“Polynucleotide,” “nucleic acid,” and “nucleic acid molecule,” are used interchangeably herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. The terms can be used to describe a polymer of any length, e.g., greater than about 2 bases, greater than about 10 bases, greater than about 100 bases, greater than about 500 bases, greater than 1000 bases, or up to about 10,000 or more bases, composed of nucleotides, e.g., deoxyribonucleotides or ribonucleotides, and may be produced enzymatically or synthetically (e.g., as described in U.S. Pat. No. 5,948,902 and the references cited therein), which can hybridize with naturally occurring nucleic acids in a sequence specific manner analogous to that of two naturally occurring nucleic acids, e.g., can participate in Watson-Crick base pairing interactions. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 06-methylguanine, 4-thio-pyrimidines, 4-amino-pyrimidines, 4-dimethylhydrazine-pyrimidines, and 04-alkyl-pyrimidines; U.S. Pat. No. 5,378,825 and PCT No. WO 93/13121). For general discussion see The Biochemistry of the Nucleic Acids 5-36, Adams et al., ed., 11th ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). All nucleotide sequences disclosed herein can represent an RNA sequence or a corresponding DNA sequence. It is understood that deoxythymidine (dT or T) in a DNA is transcribed into a uridine (U) in an RNA. As such, “T” and “U” are used interchangeably herein in nucleotide sequences.

An “oligonucleotide” is a polynucleotide comprising fewer than 1000 nucleotides, such as a polynucleotide comprising fewer than 500 nucleotides or fewer than 100 nucleotides.

As used herein, the terms “monotron,” “monotron sequence,” or “monotron element” are used interchangeably to refer a segment of a precursor RNA polynucleotide that is located at either the 5′ or 3′ end of the polynucleotide, i.e., either 5′ or 3′ from the intervening region. A monotron element refers to a sequence with 70% or higher similarity to a natural group I or group II intron including the splice site dinucleotide. In some embodiments, the monotron is capable of contributing to ribozymatic activity that allows it to enzymatically self-cleave. In some embodiments, the monotron is capable of forming a phosphodiester bond with a terminal sequence, i.e., a sequence containing a splice site dinucleotide and optionally a natural exon sequence or fragment thereof. In some embodiments, the terminal sequence is upstream of the monotron in a linear precursor. In some embodiments, the monotron sequence is upstream of the terminal sequence in a linear precursor. When the terminal sequence is upstream to the monotron in a linear precursor, the monotron can perform two transesterification reactions, e.g., sequentially, self-cleavage and formation of a phosphodiester bond with the terminal sequence. In embodiments in which the terminal sequence is upstream to the monotron in the linear precursor, (a) the monotron is capable of interacting with a nucleophile that is capable of cleaving at the splice site dinucleotide at or near the 5′ end of the monotron, and (b) the cleavage product of (a), i.e., the 5′ splice site nucleotide, e.g., having a 3′ hydroxyl group, engages in a transesterification reaction (cleaves) at the splice site nucleotide of the terminal sequence, yielding a circular RNA or ORNA. In these embodiments, the monotron interacts with the nucleophile (e.g., a guanosine, e.g., a free guanosine that is introduced to the precursor) by forming a binding pocket with the nucleophile, and the linear precursor is capable of adopting a conformation in which the nucleophile is in proximity to and is capable of cleaving at the splice site dinucleotide at or near the 5′ end of the monotron. When the monotron is upstream of the terminal sequence in a linear precursor, the monotron can also perform two transesterification reactions. In embodiments in which the monotron is upstream of the terminal sequence in the linear precursor, (a) the monotron is capable of interacting with a nucleophile that is capable of cleaving at the splice site nucleotide of the terminal element, and (b) the cleavage product of (a), i.e., the 5′ splice site nucleotide, e.g., having a 3′ hydroxyl group, engages in a transesterification reaction (cleaves) at the splice site dinucleotide at or near the 3′ end of the monotron, yielding a circular RNA or oRNA. In these embodiments, the monotron interacts with the nucleophile (e.g., a guanosine, e.g., a free guanosine that is introduced to the precursor) by forming a binding pocket with the nucleophile, and the linear precursor is capable of adopting a conformation in which the nucleophile is in proximity to and is capable of cleaving the splice site nucleotide of the terminal element.

In some embodiments, the monotron comprises a 5′ proximal end of a natural group I or group II intron including the splice site dinucleotide and optionally a natural exon sequence or fragment thereof. In some embodiments, the 5′ end of the monotron refers to nucleotides within the 5′ half of the monotron. In some embodiments, the 3′ end of the monotron refers to nucleotides within the 3′ half of the monotron. In some embodiments, at or near the 5′ end of the monotron refers to within the 5′ half of the monotron. In some embodiments, at or near the 5′ end of the monotron refers to within the first ten 5′ positions in the monotron. In some embodiments, at the 5′ end of the monotron refers to the first 5′ position(s) in the monotron. In some embodiments, at or near the 3′ end of the monotron refers to within the 3′ half of the monotron. In some embodiments, at or near the 3′ end of the monotron refers to within the last ten 3′ positions in the monotron. In some embodiments, at the 3′ end of the monotron refers to last 3′ position(s) in the monotron.

As used herein, the term “terminal sequence” or “terminal element” are used interchangeably to refer to an RNA sequence capable of complexing with a monotron sequence or monotron element. The terminal sequence comprises a splice site nucleotide from the natural group I or group II intron present in the monotron. In some embodiments, the terminal sequence further comprises a natural exon or a fragment thereof and/or a synthetic sequence.

The term “nucleophile” refers to a nucleophilic nucleotide or nucleoside capable of initiating a nucleophilic attack at a splice site and/or transesterification reaction (cleavage) at a splice site.

As used herein, “polyA” means a polynucleotide or a portion of a polynucleotide consisting of nucleotides comprising adenine. As used herein, “polyT” means a polynucleotide or a portion of a polynucleotide consisting of nucleotides comprising thymine. As used herein, “polyAC” means a polynucleotide or a portion of a polynucleotide consisting of nucleotides comprising adenine or cytosine.

“Isolated” or “purified” generally refers to isolation of a substance (for example, in some embodiments, a compound, a polynucleotide, a protein, a polypeptide, a polynucleotide composition, or a polypeptide composition) such that the substance comprises a significant percent (e.g., greater than 1%, greater than 2%, greater than 5%, greater than 10%, greater than 20%, greater than 50%, or more, usually up to about 90%-100%) of the sample in which it resides. In certain embodiments, a substantially purified component comprises at least 50, 60, 70, 75, 80, 85, 90, 95, 96, 97, 98, or 99% of the sample. In additional embodiments, a substantially purified component comprises about, 80%-85%, or 90%-95%, 95-99%, 96-99%, 97-99%, or 95-100% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is more than as it is found naturally.

As used herein, “unstructured” with regard to RNA refers to an RNA sequence that is not predicted by RNA structure predictive tools to form a structure (e.g., a hairpin loop) with itself or other sequences in the same RNA molecule. In some embodiments, unstructured RNA can be functionally characterized using nuclease protection assays.

As used herein, “structured” with regard to RNA refers to an RNA sequence that is predicted by the RNAFold software or similar predictive tools to form a structure (e.g., a hairpin loop) with itself or other sequences in the same RNA molecule.

As used herein, two “duplex sequences,” “duplex region,” “duplex regions,” “homology arms,” or “homology regions” may be any two regions that are thermodynamically favored to cross-pair in a sequence specific interaction. In some embodiments, two duplex sequences, duplex regions, homology arms, or homology regions, share a sufficient level of sequence identity to one another's reverse complement to act as substrates for a hybridization reaction. As used herein, polynucleotide sequences have “homology” when they are either identical or share sequence identity to a reverse complement or “complementary” sequence. The percent sequence identity between a homology region and a counterpart homology region's reverse complement can be any percent of sequence identity that allows for hybridization to occur. In some embodiments, an internal duplex region of an inventive polynucleotide is capable of forming a duplex with another internal duplex region and does not form a duplex with an external duplex region.

As used herein, an “affinity sequence” or “affinity tag” is a region of polynucleotide sequences polynucleotide sequence ranging from 1 nucleotide to hundreds or thousands of nucleotides containing a repeated set of nucleotides for the purposes of aiding purification of a polynucleotide sequence. For example, an affinity sequence may comprise, but is not limited to, a polyA or polyAC sequence. In some embodiments, affinity tags are used in purification methods, referred to herein as “affinity-purification,” in which selective binding of a binding agent to molecules comprising an affinity tag facilitates separation from molecules that do not comprise an affinity tag. In some embodiments, an affinity-purification method is a “negative selection” purification method, in which unwanted species, such as linear RNA, are selectively bound and removed and wanted species, such as circular RNA, are eluted and separated from unwanted species.

As used herein, a “spacer” refers to a region of a polynucleotide sequence ranging from 1 nucleotide to hundreds or thousands of nucleotides separating two other elements along a polynucleotide sequence. The sequences can be defined or can be random. A spacer is typically non-coding. In some embodiments, spacers include duplex regions.

Linear nucleic acid molecules are said to have a “5′-terminus” (5′ end) and a “3′-terminus” (3′ end) because nucleic acid phosphodiester linkages occur at the 5′ carbon and 3′ carbon of the sugar moieties of the substituent mononucleotides. The end nucleotide of a polynucleotide at which a new linkage would be to a 5′ carbon is its 5′ terminal nucleotide. The end nucleotide of a polynucleotide at which a new linkage would be to a 3′ carbon is its 3′ terminal nucleotide. A terminal nucleotide, as used herein, is the nucleotide at the end position of the 3′- or 5′-terminus.

As used herein, a “leading untranslated sequence” is a region of polynucleotide sequences ranging from 1 nucleotide to hundreds of nucleotides located at the upmost 5′ end of a polynucleotide sequence. The sequences can be defined or can be random. A leading untranslated sequence is non-coding.

As used herein, a “terminal untranslated sequence” is a region of polynucleotide sequences ranging from 1 nucleotide to hundreds of nucleotides located at the downmost 3′ end of a polynucleotide sequence. The sequences can be defined or can be random. A terminal untranslated sequence is non-coding.

“Transcription” means the formation or synthesis of an RNA molecule by an RNA polymerase using a DNA molecule as a template. The disclosure is not limited with respect to the RNA polymerase that is used for transcription. For example, in some embodiments, a T7-type RNA polymerase can be used.

“Translation” means the formation of a polypeptide molecule by a ribosome based upon an RNA template.

As used herein, an “internal ribosome entry site” or “IRES” refers to an RNA sequence or structural element ranging in size from 10 nt to 1000 nt or more, capable of initiating translation of a polypeptide in the absence of a typical RNA cap structure. An exemplary IRES can be about 500 nt to about 700 nt in length.

As used herein, the terms “about” or “approximately” means an acceptable error for a particular value as determined by one of ordinary skill in the art, which depends in part on how the value is measured or determined. Unless specifically stated or obvious from context, as used herein, the term “about,” is understood as within a range of normal tolerance in the art, for example within 2 standard deviations of the mean. Unless otherwise clear from the context, all numerical values provided herein are modified by the term “about.”

As used herein, the term “encode” refers broadly to any process whereby the information in a polymeric macromolecule is used to direct the production of a second molecule that is different from the first. The second molecule may have a chemical structure that is different from the chemical nature of the first molecule.

As used herein, “aptamer” refers in general to either an oligonucleotide of a single defined sequence or a mixture of said nucleotides, wherein the mixture retains the properties of binding specifically to the target molecule (e.g., eukaryotic initiation factor, 40S ribosome, polyC binding protein, polyA binding protein, polypyrimidine tract-binding protein, argonaute protein family, Heterogeneous nuclear ribonucleoprotein K and La and related RNA-binding protein). Thus, as used herein “aptamer” denotes both singular and plural sequences of nucleotides, as defined hereinabove. The term “aptamer” is meant to refer to a single- or double-stranded nucleic acid which is capable of binding to a protein or other molecule. In general, aptamers preferably comprise about 10 to about 100 nucleotides, preferably about 15 to about 40 nucleotides, more preferably about 20 to about 40 nucleotides, in that oligonucleotides of a length that falls within these ranges are readily prepared by conventional techniques. Optionally, aptamers can further comprise a minimum of approximately 6 nucleotides, preferably 10, and more preferably 14 or 15 nucleotides, that are necessary to effect specific binding.

As used herein, a “miRNA site” or “miRNA binding site” refers to a stretch of nucleotides within a polynucleotide that is capable of forming a duplex with at least 8 nucleotides of a natural miRNA sequence.

As used herein, “bicistronic RNA” refers to a polynucleotide that includes two expression sequences coding for two distinct proteins. These expression sequences can be separated by a nucleotide sequence encoding a cleavable peptide such as a protease cleavage site. They can also be separated by a ribosomal skipping element.

As used herein, the term “ribosomal skipping element” refers to a nucleotide sequence encoding a short peptide sequence capable of causing generation of two peptide chains from translation of one RNA molecule. While not wishing to be bound by theory, it is hypothesized that ribosomal skipping elements function by (1) terminating translation of the first peptide chain and re-initiating translation of the second peptide chain; or (2) cleavage of a peptide bond in the peptide sequence encoded by the ribosomal skipping element by an intrinsic protease activity of the encoded peptide, or by another protease in the environment (e.g., cytosol).

As used herein, the terms “transfect” or “transfection” refer to the intracellular introduction of one or more encapsulated materials (e.g., nucleic acids and/or polynucleotides) into a cell, or preferably into a target cell. The term “transfection efficiency” refers to the relative amount of such encapsulated material (e.g., polynucleotides) up-taken by, introduced into and/or expressed by the target cell which is subject to transfection. In some embodiments, transfection efficiency may be estimated by the amount of a reporter polynucleotide product produced by the target cells following transfection. In some embodiments, a transfer vehicle has high transfection efficiency. In some embodiments, a transfer vehicle has at least about 10%, 20%, 30%, 40%, 50%, 60%, 70%, 80%, or 90% transfection efficiency.

As used herein, “transfer vehicle” includes any of the standard pharmaceutical carriers, diluents, excipients, and the like, which are generally intended for use in connection with the administration of biologically active agents, including nucleic acids.

As used herein, the phrase “nanoparticle” refers to a delivery or transfer vehicle, for example, having a diameter of less than about 1000 nm. A nanoparticle can be a “lipid nanoparticle,” and in certain instances herein, the terms are used interchangeably herein.

As used herein, the phrase “LNP” or “lipid nanoparticle” refers to a delivery or transfer vehicle comprising one or more cationic or ionizable lipids, stabilizing lipids, structural lipids, and helper lipids.

As used herein, the phrase “cationic lipid” or “ionizable lipid” refers to any of a number of lipid species that carry a net positive charge at a selected pH, such as physiological pH 4 and a neutral charge at other pHs such as physiological pH 7.

In some embodiments, a lipid, e.g., an ionizable lipid, disclosed herein comprises one or more cleavable groups. The terms “cleave” and “cleavable” are used herein to mean that one or more chemical bonds (e.g., one or more of covalent bonds, hydrogen-bonds, van der Waals' forces and/or ionic interactions) between atoms in or adjacent to the subject functional group are broken (e.g., hydrolyzed) or are capable of being broken upon exposure to selected conditions (e.g., upon exposure to enzymatic conditions). In certain embodiments, the cleavable group is a disulfide functional group, and in particular embodiments is a disulfide group that is capable of being cleaved upon exposure to selected biological conditions (e.g., intracellular conditions). In certain embodiments, the cleavable group is an ester functional group that is capable of being cleaved upon exposure to selected biological conditions. For example, the disulfide groups may be cleaved enzymatically or by a hydrolysis, oxidation or reduction reaction. Upon cleavage of such disulfide functional group, the one or more functional moieties or groups (e.g., one or more of a head-group and/or a tail-group) that are bound thereto may be liberated. Exemplary cleavable groups may include, but are not limited to, disulfide groups, ester groups, ether groups, and any derivatives thereof (e.g., alkyl and aryl esters). In certain embodiments, the cleavable group is not an ester group or an ether group. In some embodiments, a cleavable group is bound (e.g., bound by one or more of hydrogen-bonds, van der Waals' forces, ionic interactions and covalent bonds) to one or more functional moieties or groups (e.g., at least one head-group and at least one tail-group). In certain embodiments, at least one of the functional moieties or groups is hydrophilic (e.g., a hydrophilic head-group comprising one or more of imidazole, guanidinium, amino, imine, enamine, optionally-substituted alkyl amino and pyridyl).

As used herein, the term “liposome” generally refers to a vesicle composed of lipids (e.g., amphiphilic lipids) arranged in one or more spherical bilayer or bilayers. Such liposomes may be unilamellar or multilamellar vesicles which have a membrane formed from a lipophilic material and an aqueous interior that contains the encapsulated circRNA to be delivered to one or more target cells, tissues and organs. In certain embodiments, the compositions described herein comprise one or more lipid nanoparticles. Examples of suitable lipids (e.g., ionizable lipids) that may be used to form the liposomes and lipid nanoparticles contemplated include one or more of the compounds disclosed herein (e.g., HGT4001, HGT4002, HGT4003, HGT4004 and/or HGT4005). Such liposomes and lipid nanoparticles may also comprise additional ionizable lipids such as C12-200, DLin-KC2-DMA, and/or HGT5001, helper lipids, structural lipids, PEG-modified lipids, MC3, DLinDMA, DLinkC2DMA, cKK-E12, ICE, HGT5000, DODAC, DDAB, DMRIE, DOSPA, DOGS, DODAP, DODMA, DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, HGT4003, and combinations thereof.

As used herein, the phrase “biodegradable lipid” or “degradable lipid” refers to any of a number of lipid species that are broken down in a host environment on the order of minutes, hours, or days ideally making them less toxic and unlikely to accumulate in a host over time. Common modifications to lipids include ester bonds, and disulfide bonds among others to increase the biodegradability of a lipid.

As used herein, the term “structural lipid” refers to sterols and also to lipids containing sterol moieties.

As defined herein, “sterols” are a subgroup of steroids consisting of steroid alcohols.

As used herein, the term “PEG” means any polyethylene glycol or other polyalkylene ether polymer. As generally defined herein, a “PEG-OH lipid” (also referred to herein as “hydroxy-PEGylated lipid”) is a PEGylated lipid having one or more hydroxyl (—OH) groups on the lipid. As used herein, the phrase “biodegradable PEG lipid” or “degradable PEG lipid” refers to any of a number of lipid species where the PEG molecules are cleaved from the lipid in a host environment on the order of minutes, hours, or days ideally making them less immunogenic. Common modifications to PEG lipids include ester bonds, and disulfide bonds among others to increase the biodegradability of a lipid.

As used herein, the term “hydrophilic” is used to indicate in qualitative terms that a functional group is water-preferring, and typically such groups are water-soluble. For example, disclosed herein are compounds that comprise a cleavable disulfide (S—S) functional group bound to one or more hydrophilic groups (e.g., a hydrophilic head-group), wherein such hydrophilic groups comprise or are selected from the group consisting of imidazole, guanidinium, amino, imine, enamine, an optionally-substituted alkyl amino(e.g., an alkyl amino such as dimethylamino) and pyridyl.

In certain embodiments, at least one of the functional groups of moieties that comprise the compounds disclosed herein is hydrophobic in nature (e.g., a hydrophobic tail-group comprising a naturally occurring lipid such as cholesterol). As used herein, the term “hydrophobic” is used to indicate in qualitative terms that a functional group is water-avoiding, and typically such groups are not water soluble. For example, disclosed herein are compounds that comprise a cleavable functional group (e.g., a disulfide (S—S) group) bound to one or more hydrophobic groups, wherein such hydrophobic groups comprise one or more naturally occurring lipids such as cholesterol, and/or an optionally substituted, variably saturated or unsaturated C6-C20 alkyl and/or an optionally substituted, variably saturated or unsaturated C6-C20 acyl.

Compounds described herein may also comprise one or more isotopic substitutions. For example, H may be in any isotopic form, including 1H, 2H (D or deuterium), and 3H (T or tritium); C may be in any isotopic form, including 12C, 13C, and 14C; O may be in any isotopic form, including 160 and 180; F may be in any isotopic form, including 18F and 19F; and the like.

As used herein, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

When a range of values is listed, it is intended to encompass each value and sub-range within the range. For example, “C1-6 alkyl” is intended to encompass, C1, C2, C3, C4, C5, C6, C1-6, C1-5, C1-4, C1-3, C1-2, C2-6, C2-5, C2-4, C2-3, C3-6, C3-5, C3-4, C4-6, C4-5, and C5-6 alkyl.

As used herein, the term “alkyl” refers to both straight and branched chain C1-C40 hydrocarbons (e.g., C6-C20 hydrocarbons), and include both saturated and unsaturated hydrocarbons. In certain embodiments, the alkyl may comprise one or more cyclic alkyls and/or one or more heteroatoms such as oxygen, nitrogen, or sulfur and may optionally be substituted with substituents (e.g., one or more of alkyl, halo, alkoxyl, hydroxy, amino, aryl, ether, ester or amide). In certain embodiments, a contemplated alkyl includes (9Z,12Z)-octadeca-9,12-dien. The use of designations such as, for example, “C6-C20” is intended to refer to an alkyl (e.g., straight or branched chain and inclusive of alkenes and alkyls) having the recited range carbon atoms. In some embodiments, an alkyl group has 1 to 10 carbon atoms (“C1-10 alkyl”). In some embodiments, an alkyl group has 1 to 9 carbon atoms (“C1-9 alkyl”). In some embodiments, an alkyl group has 1 to 8 carbon atoms (“C1-8 alkyl”). In some embodiments, an alkyl group has 1 to 7 carbon atoms (“C1-7 alkyl”). In some embodiments, an alkyl group has 1 to 6 carbon atoms (“C1-6 alkyl”). In some embodiments, an alkyl group has 1 to 5 carbon atoms (“C1-5 alkyl”). In some embodiments, an alkyl group has 1 to 4 carbon atoms (“C1-4 alkyl”). In some embodiments, an alkyl group has 1 to 3 carbon atoms (“C1-3 alkyl”). In some embodiments, an alkyl group has 1 to 2 carbon atoms (“C1-2 alkyl”). In some embodiments, an alkyl group has 1 carbon atom (“C1 alkyl”). Examples of C1-6 alkyl groups include methyl, ethyl, propyl, isopropyl, butyl, isobutyl, pentyl, hexyl, and the like.

As used herein, “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C2-20 alkenyl”). In certain embodiments, alkenyl does not contain any triple bonds. In some embodiments, an alkenyl group has 2 to 10 carbon atoms (“C2-10 alkenyl”). In some embodiments, an alkenyl group has 2 to 9 carbon atoms (“C2-9 alkenyl”). In some embodiments, an alkenyl group has 2 to 8 carbon atoms (“C2-8 alkenyl”). In some embodiments, an alkenyl group has 2 to 7 carbon atoms (“C2-7 alkenyl”). In some embodiments, an alkenyl group has 2 to 6 carbon atoms (“C2-6 alkenyl”). In some embodiments, an alkenyl group has 2 to 5 carbon atoms (“C2-5 alkenyl”). In some embodiments, an alkenyl group has 2 to 4 carbon atoms (“C2-4 alkenyl”). In some embodiments, an alkenyl group has 2 to 3 carbon atoms (“C2-3 alkenyl”). In some embodiments, an alkenyl group has 2 carbon atoms (“C2 alkenyl”). The one or more carbon-carbon double bonds can be internal (such as in 2-butenyl) or terminal (such as in 1-butenyl). Examples of C2-4 alkenyl groups include ethenyl (C2), 1-propenyl (C3), 2-propenyl (C3), 1-butenyl (C4), 2-butenyl (C4), butadienyl (C4), and the like. Examples of C2-6 alkenyl groups include the aforementioned C2-4 alkenyl groups as well as pentenyl (C5), pentadienyl (C5), hexenyl (C6), and the like. Additional examples of alkenyl include heptenyl (C7), octenyl (C8), octatrienyl (C8), and the like.

As used herein, the term “aryl” refers to aromatic groups (e.g., monocyclic, bicyclic and tricyclic structures) containing six to ten carbons in the ring portion. The aryl groups may be optionally substituted through available carbon atoms and in certain embodiments may include one or more heteroatoms such as oxygen, nitrogen or sulfur. In some embodiments, an aryl group has six ring carbon atoms (“C6 aryl”; e.g., phenyl). In some embodiments, an aryl group has ten ring carbon atoms (“C10 aryl”; e.g., naphthyl such as 1-naphthyl and 2-naphthyl).

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” may be used interchangeably.

As used herein, “cyano” refers to —CN.

The terms “halo” and “halogen” as used herein refer to an atom selected from fluorine (fluoro, F), chlorine (chloro, Cl), bromine (bromo, Br), and iodine (iodo, I). In certain embodiments, the halo group is either fluoro or chloro.

The term “alkoxy,” as used herein, refers to an alkyl group which is attached to another moiety via an oxygen atom (—O(alkyl)). Non-limiting examples include e.g., methoxy, ethoxy, propoxy, and butoxy.

As used herein, “oxo” refers to —C═O.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, ethanesulfonate, dodecylsulfate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N+(C1-4alkyl)4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “composition” or “formulation” refers to a preparation which is in such form as to permit the biological activity of an active ingredient contained therein to be effective, and which contains no additional components which are unacceptably toxic to a subject to which the composition would be administered.

As used herein, “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, may serve as an antigen. An antigen may be endogenously expressed, i.e., expressed by genomic DNA, or may be recombinantly expressed. An antigen may be specific to a certain tissue, such as a cancer cell, or it may be broadly expressed. In addition, fragments of larger molecules may act as antigens. In some embodiments, antigens are tumor antigens.

As used herein, “treatment” (and variations thereof such as “treat” or “treating”) refers to any administration or application of a therapeutic for disease or disorder in a subject, and includes inhibiting the disease or development of the disease (which may occur before or after the disease is formally diagnosed, e.g., in cases where a subject has a genotype that has the potential or is likely to result in development of the disease), arresting its development, relieving one or more symptoms of the disease, curing the disease, or preventing reoccurrence of one or more symptoms of the disease. As used herein, “treatment” can include administrating a therapeutic or therapeutic regimen including optional adjuvant or pre-conditioning regimen to achieve a therapeutic or prophylactic benefit. As used herein, “treatment” also encompasses “ameliorating,” which refers to any beneficial effect on a phenotype or symptom, such as reducing its severity, slowing or delaying its development, arresting its development, or partially or completely reversing or eliminating it.

As used herein, “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. Examples of cancers that may be treated by the methods disclosed herein include, but are not limited to, cancers of the immune system including lymphoma, leukemia, myeloma, and other leukocyte malignancies. In some embodiments, the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, neoplasm of the central nervous system (CNS), primary CNS lymphoma, tumor angiogenesis, spinal axis tumor, brain stem glioma, pituitary adenoma, epidermoid cancer, squamous cell cancer, T cell lymphoma, environmentally induced cancers including those induced by asbestos, other B cell malignancies, and combinations of said cancers. In some embodiments, the methods disclosed herein may be used to reduce the tumor size of a tumor derived from, for example, sarcomas and carcinomas, fibrosarcoma, myxosarcoma, liposarcoma, chondrosarcoma, osteogenic sarcoma, Kaposi's sarcoma, sarcoma of soft tissue, and other sarcomas, synovioma, mesothelioma, Ewing's tumor, leiomyosarcoma, rhabdomyosarcoma, colon carcinoma, pancreatic cancer, breast cancer, ovarian cancer, prostate cancer, hepatocellular carcinoma, lung cancer, colorectal cancer, squamous cell carcinoma, basal cell carcinoma, adenocarcinoma (for example adenocarcinoma of the pancreas, colon, ovary, lung, breast, stomach, prostate, cervix, or esophagus), sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, carcinoma of the fallopian tubes, carcinoma of the endometrium, carcinoma of the cervix, carcinoma of the vagina, carcinoma of the vulva, carcinoma of the renal pelvis, CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma). The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

As used herein, an “autoimmune disease” refers to a disease or disorder directed against and/or arising from a subject's own tissues and/or organs. Clinical and laboratory markers of autoimmune disease are known in the art. Exemplary markers include, but are not limited to, high levels of autoantibodies, antigen-antibody complex deposits (e.g., in the subject's tissue(s)), lymphoid cell aggregates in affected tissues, hypergammaglobulinemia. Exemplary autoimmune diseases include, but are not limited to, lupus, e.g., systemic lupus erythematosus (SLE), cutaneous lupus erythematosus (CLE), lupus nephritis (LN), antisynthetase syndrome, multifocal motor neuropathy, myasthenia gravis, neuromyelitis optica, pemphigus vulgaris, and systemic sclerosis. In some embodiments, the autoimmune disease is one that is B-cell mediated. Autoimmunity may be associated with autoantibody production, immune complex formation, dendritic cell activation, T cell activation, cytokine synthesis, and/or chemokine release. For example, SLE “is a life-threatening autoimmune disease characterized by adaptive immune system activation, formation of double-stranded DNA autoantibodies and organ inflammation.” Mackensen et al., Anti-CD19 CAR T cell therapy for refractory systemic lupus erythematosus, Nature Medicine (2022). SLE may be assessed using the Systemic Lupus Erythematosus Disease Activity Index and/or DORIS criteria. Id.

An “anti-tumor effect” as used herein, refers to a biological effect that may present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect may also refer to the prevention of the occurrence of a tumor, e.g., a vaccine.

As used herein, the term “administering” refers to the physical introduction of an agent to a subject, using any of the various methods and delivery systems known to those skilled in the art. Exemplary routes of administration for the agents disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the agents disclosed herein may be administered via a non-parenteral route, e.g., orally. Other non-parenteral routes include a topical, epidermal, or mucosal route of administration, for example, intranasally, vaginally, rectally, sublingually or topically. The phrase “systemic injection” as used herein non-exclusively relates to intravenous, intraperitoneally, subcutaneous, via nasal submucosa, lingual, via bronchoscopy, intravenous, intra-arterial, intra-muscular, intro-ocular, intra-striatal, subcutaneous, intradermal, by dermal patch, by skin patch, by patch, into the cerebrospinal fluid, into the portal vein, into the brain, into the lymphatic system, intra-pleural, retro-orbital, intra-dermal, into the spleen, intra-lymphatic, among others.

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

A “cytokine,” as used herein, refers to a non-antibody protein that is released by one cell in response to contact with a specific antigen, wherein the cytokine interacts with a second cell to mediate a response in the second cell. “Cytokine” as used herein is meant to refer to proteins released by one cell population that act on another cell as intercellular mediators. A cytokine may be endogenously expressed by a cell or administered to a subject. Cytokines may be released by immune cells, including macrophages, B cells, T cells, neutrophils, dendritic cells, eosinophils and mast cells to propagate an immune response. Cytokines may induce various responses in the recipient cell. Cytokines may include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-la, IL-1b, IL-6, IL-13, IL-17a, IL-23, IL-27, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), TGF-beta, IL-35, and perforin. Examples of acute phase-proteins include, but are not limited to, C-reactive protein (CRP) and serum amyloid A (SAA).

The term “lymphocyte” as used herein includes natural killer (NK) cells, T cells, or B cells. NK cells are a type of cytotoxic (cell toxic) lymphocyte that represent a major component of the innate immune system. NK cells reject tumors and cells infected by viruses. It works through the process of apoptosis or programmed cell death. They were termed “natural killers” because they do not require activation in order to kill cells. T cells play a major role in cell-mediated-immunity (no antibody involvement). T cell receptors (TCR) differentiate T cells from other lymphocyte types. The thymus, a specialized organ of the immune system, is the primary site for T cell maturation. There are numerous types of T cells, including: helper T cells (e.g., CD4+ cells), cytotoxic T cells (also known as TC, cytotoxic T lymphocytes, CTL, T-killer cells, cytolytic T cells, CD8+ T cells or killer T cells), memory T cells ((i) stem memory cells (TSCM), like naive cells, are CD45RO−, CCR7+, CD45RA+, CD62L+ (L-selectin), CD27+, CD28+ and IL-7Rₐ+, but also express large amounts of CD95, IL-2R, CXCR3, and LFA-1, and show numerous functional attributes distinctive of memory cells); (ii) central memory cells (TCM) express L-selectin and CCR7, they secrete IL-2, but not IFNγ or IL-4, and (iii) effector memory cells (TEM), however, do not express L-selectin or CCR7 but produce effector cytokines like IFNγ and IL-4), regulatory T cells (Tregs, suppressor T cells, or CD4+CD25+ or CD4+FoxP3+ regulatory T cells), natural killer T cells (NKT) and gamma delta T cells. B-cells, on the other hand, play a principal role in humoral immunity (with antibody involvement). B-cells make antibodies, are capable of acting as antigen-presenting cells (APCs), and turn into memory B-cells and plasma cells, both short-lived and long-lived, after activation by antigen interaction. In mammals, immature B-cells are formed in the bone marrow.

An “immune response” refers to the action of a cell of the immune system (for example, T lymphocytes, B lymphocytes, natural killer (NK) cells, macrophages, cosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

A “costimulatory signal,” as used herein, refers to a signal, which in combination with a primary signal, such as TCR/CD3 ligation, leads to a T cell response, such as, but not limited to, proliferation and/or upregulation or down regulation of key molecules.

A “costimulatory ligand,” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand may include, but is not limited to, 3/TR6, 4—IBB ligand, agonist or antibody that binds Toll-like receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) LI. A co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD 18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD1-1a, CD1-1b, CD1-1c, CD1-1d, CDS, CEACAM1, CRT AM, DAP-10, DNAMI (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, IT GAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CD1 la/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

As used herein, “subject” may be a mammal, such as a primate, ungulate (e.g., cow, pig, horse), cat, dog, domestic pet or domesticated mammal. In some cases, the mammal may be a rabbit, pig, horse, sheep, cow, cat or dog, or a human. In some embodiments, the subject is a human. In some embodiments, the subject is an adult human. In some embodiments, the subject is a juvenile human.

### II. Circular RNA and Compositions Thereof

Provided herein are circular RNA constructs and related pharmaceutical compositions comprising transfer vehicles, wherein the circular RNA constructs are capable of in vivo delivery to immune cells for therapy or production of proteins. According to the present disclosure, the circular RNA provided herein can be injected into an animal (e.g., a human), such that a polypeptide encoded by the circular RNA molecule is expressed inside the animal, for example by immune cells and T cells.

In certain embodiments, the circular RNA constructs comprise an IRES. In certain embodiments, the circular RNA constructs comprise at least one expression sequence encoding a binding molecule, wherein the binding molecule binds to or associates with a tumor cell antigen. In certain embodiments, the circular RNA constructs comprise an IRES and at least one expression sequence encoding a binding molecule.

In some embodiments, provided herein are circular RNA polynucleotides comprising a post splicing 3′ group I intron fragment (e.g., a stretch of exon sequence), optionally a first spacer, an Internal Ribosome Entry Site (IRES), an expression sequence, optionally a second spacer, and a post splicing 5′ group I intron fragment (e.g., a stretch of exon sequence). In some embodiments, these regions are in that order.

In certain embodiments, a circular RNA constructed is formulated into a pharmaceutical composition. In certain embodiments, the pharmaceutical composition comprises a transfer vehicle. In certain embodiments, a circular RNA construct comprising an IRES and at least one expression sequence encoding a binding molecule is formulated into a pharmaceutical composition comprising a transfer vehicle.

In certain embodiments, pharmaceutical compositions comprising a circular RNA construct comprising an IRES and at least one expression sequence encoding a binding molecule, and a transfer vehicle are disclosed. In certain embodiments, the transfer vehicle facilitates and/or enhances the delivery and release of circular RNA to one or more target cells.

In certain embodiments, the circular RNA constructs and related pharmaceutical compositions comprise an IRES and at least one expression sequence encoding a therapeutic protein, wherein the IRES is capable of facilitating expression of the protein when delivered in vivo.

In certain embodiments, the circular RNA constructs comprise an IRES and at least one expression sequence encoding a cytokine, immune checkpoint inhibitor, agonist, chimeric antigen receptor (CAR), inhibitory receptor agonist, one or more T-Cell Receptors, and/or B-cell Receptors.

In some embodiments, a polynucleotide encodes a protein that is made up of subunits that are encoded by more than one gene. For example, the protein may be a heterodimer, wherein each chain or subunit of the protein is encoded by a separate gene. It is possible that more than one circular RNA molecule is delivered in the transfer vehicle and each circular RNA encodes a separate subunit of the protein. Alternatively, a single circular RNA may be engineered to encode more than one subunit. In certain embodiments, separate circular RNA molecules encoding the individual subunits may be administered in separate transfer vehicles.

In certain embodiments, the circular RNA comprises an IRES and at least one expression sequence encoding a CAR construct. In some embodiments, the CAR targets a cancer antigen. In some embodiments, the CAR may be programmed to both recognize a specific antigen and, when bound to that antigen, activate the immune cell to attack and destroy the cell. In certain embodiments, the payload encoded by the circular RNA polynucleotide may be optimized through use of a specific internal ribosome entry sites (IRES) within the translation initiation element (TIE). The TIE can comprise an untranslated region (UTR), aptamer complex, or a combination thereof. The UTR can be in whole or in part from a viral or eukaryotic mRNA. In some embodiments, IRES specificity within a circular RNA can significantly enhance expression of specific proteins encoded within the coding element.

The circular RNA is produced by transcription of a DNA template that results in formation of a precursor linear RNA polynucleotide capable of circularizing. Linear precursor RNA polynucleotides are provided for producing circular RNA constructs and related pharmaceutical compositions. The DNA template shares the same sequence as the precursor linear RNA polynucleotide prior to splicing of the precursor linear RNA polynucleotide. The DNA template shares the same sequence as the precursor linear RNA polynucleotide prior to splicing of the precursor linear RNA polynucleotide (e.g., a 3′ enhanced intron element, a 3′ enhanced exon element, a core functional element, and a 5′ enhanced exon element, a 5′ enhanced intron element). In some embodiments, said linear precursor RNA polynucleotide undergoes splicing leading to the removal of the 3′ enhanced intron element and 5′ enhanced intron element during the process of circularization. In some embodiments, the resulting circular RNA polynucleotide lacks a 3′ enhanced intron fragment and a 5′ enhanced intron fragment, but maintains a 3′ enhanced exon fragment, a core functional element, and a S′ enhanced exon element. Circularization strategies are known in the art and described elsewhere herein. In certain embodiments, the resulting circular RNA can include a PIE (permuted intron-exon) region, a translation region (IRES and coding/noncoding elements), and a PIE region. The resulting permuted intron-exon (PIE) regions allow for 5′ and 3′ ends of the RNA to covalently link and form the circular RNA.

In some embodiments, the precursor RNA polynucleotide comprises, in the following order, (a) a terminal element; (b) an intervening region, and (c) a monotron element. In some embodiments, the terminal sequence is upstream of the monotron sequence in the precursor RNA polynucleotide. In such embodiments: (i) the terminal element comprises a splice site nucleotide, (ii) the monotron element comprises a splice site dinucleotide at or near the 5′ end of the monotron, and (iii) the monotron element is capable of interacting with a nucleophile that is capable of cleaving at the splice site dinucleotide at or near the 5′ end of the monotron, where the cleavage product of (iii) comprises a 5′ splice site nucleotide that is capable of cleaving at the splice site nucleotide of the terminal element. In some embodiments, the nucleophile is a free nucleophile that is introduced to the precursor RNA polynucleotide, e.g., not in cis and/or covalently linked to the precursor RNA polynucleotide. In some embodiments, the nucleophile is a guanosine that is capable of cleaving at the splice site dinucleotide at or near the 5′ end of the monotron. In some embodiments, the guanosine is a free guanosine that is introduced to the precursor RNA polynucleotide, e.g., not in cis and/or covalently linked to the precursor RNA polynucleotide. In some embodiments, the cleavage product of (iii) comprises a 5′ splice site nucleotide having a 3′ hydroxyl group that is capable of cleaving at the splice site nucleotide of the terminal element.

In some embodiments, the precursor RNA polynucleotide comprises, in the following order, (a) a monotron element; (b) an intervening region, and (c) terminal element. In some embodiments, the monotron sequence is upstream of the terminal sequence in the precursor RNA polynucleotide. In such embodiments: (i) the monotron element comprises a splice site dinucleotide at or near the 3′ end of the monotron, (ii) the terminal element comprises a splice site nucleotide, and (iii) the monotron element is capable of interacting with a nucleophile that is capable of cleaving at the splice site nucleotide of the terminal element, where the cleavage product of (iii) comprises a 5′ splice site nucleotide that is capable of cleaving at the splice site dinucleotide at or near the 3′ end of the monotron. In some embodiments, the nucleophile is a free nucleophile that is introduced to the precursor RNA polynucleotide, e.g., not in cis and/or covalently linked to the precursor RNA polynucleotide. In some embodiments, the nucleophile is a guanosine that is capable of cleaving at the splice site nucleotide of the terminal element. In some embodiments, the guanosine is a free guanosine that is introduced to the precursor RNA polynucleotide, e.g., not in cis and/or covalently linked to the precursor RNA polynucleotide. In some embodiments, the cleavage product of (iii) comprises a 5′ splice site nucleotide having a 3′ hydroxyl group that is capable of cleaving at the splice site nucleotide of the terminal element.

In some embodiments, the precursor linear RNA polynucleotide circularizes when incubated in the presence of one or more guanosine nucleotides or nucleoside (e.g., GTP) and a divalent cation (e.g., Mg²⁺). In some embodiments, the 3′ enhanced exon element, 5′ enhanced exon element, and/or core functional element in whole or in part promotes the circularization of the precursor linear RNA polynucleotide to form the circular RNA polynucleotide provided herein.

In certain embodiments circular RNA provided herein is produced inside a cell. In some embodiments, precursor RNA is transcribed using a DNA template (e.g., in some embodiments, using a vector provided herein) in the cytoplasm by a bacteriophage RNA polymerase, or in the nucleus by host RNA polymerase II and then circularized.

In certain embodiments, the circular RNA provided herein is injected into an animal (e.g., a human), such that a polypeptide encoded by the circular RNA molecule is expressed inside the animal.

In some embodiments, the DNA (e.g., vector), linear RNA (e.g., precursor RNA), and/or circular RNA polynucleotide provided herein is between 300 and 10000, 400 and 9000, 500 and 8000, 600 and 7000, 700 and 6000, 800 and 5000, 900 and 5000, 1000 and 5000, 1100 and 5000, 1200 and 5000, 1300 and 5000, 1400 and 5000, and/or 1500 and 5000 nucleotides in length. In some embodiments, the polynucleotide is at least 300 nt, 400 nt, 500 nt, 600 nt, 700 nt, 800 nt, 900 nt, 1000 nt, 1100 nt, 1200 nt, 1300 nt, 1400 nt, 1500 nt, 2000 nt, 2500 nt, 3000 nt, 3500 nt, 4000 nt, 4500 nt, or 5000 nt in length. In some embodiments, the polynucleotide is no more than 3000 nt, 3500 nt, 4000 nt, 4500 nt, 5000 nt, 6000 nt, 7000 nt, 8000 nt, 9000 nt, or 10000 nt in length. In some embodiments, the length of a DNA, linear RNA, and/or circular RNA polynucleotide provided herein is about 300 nt, 400 nt, 500 nt, 600 nt, 700 nt, 800 nt, 900 nt, 1000 nt, 1100 nt, 1200 nt, 1300 nt, 1400 nt, 1500 nt, 2000 nt, 2500 nt, 3000 nt, 3500 nt, 4000 nt, 4500 nt, 5000 nt, 6000 nt, 7000 nt, 8000 nt, 9000 nt, or 10000 nt.

In some embodiments, the circular RNA provided herein has higher functional stability than mRNA comprising the same expression sequence. In some embodiments, the circular RNA provided herein has higher functional stability than mRNA comprising the same expression sequence, modified nucleotides (e.g., 5moU modifications), an optimized UTR, a cap, and/or a polyA tail.

In some embodiments, the circular RNA polynucleotide provided herein has a functional half-life of at least 5 hours, 10 hours, 15 hours, 20 hours. 30 hours, 40 hours, 50 hours, 60 hours, 70 hours or 80 hours. In some embodiments, the circular RNA polynucleotide provided herein has a functional half-life of 5-80, 10-70, 15-60, and/or 20-50 hours. In some embodiments, the circular RNA polynucleotide provided herein has a functional half-life greater than (e.g., at least 1.5-fold greater than, at least 2-fold greater than) that of an equivalent linear RNA polynucleotide encoding the same protein. In some embodiments, functional half-life can be assessed through the detection of functional protein synthesis.

In some embodiments, the circular RNA polynucleotide provided herein has a half-life of at least 5 hours, 10 hours, 15 hours, 20 hours. 30 hours, 40 hours, 50 hours, 60 hours, 70 hours, or 80 hours. In some embodiments, the circular RNA polynucleotide provided herein has a half-life of 5-80, 10-70, 15-60, and/or 20-50 hours. In some embodiments, the circular RNA polynucleotide provided herein has a half-life greater than (e.g., at least 1.5-fold greater than, at least 2-fold greater than) that of an equivalent linear RNA polynucleotide encoding the same protein. In some embodiments, the circular RNA polynucleotide, or pharmaceutical composition thereof, has a functional half-life in a human cell greater than or equal to that of a pre-determined threshold value. In some embodiments the functional half-life is determined by a functional protein assay. For example in some embodiments, the functional half-life is determined by an in vitro luciferase assay, wherein the activity of Gaussia luciferase (GLuc) is measured in the media of human cells (e.g. HepG2) expressing the circular RNA polynucleotide every 1, 2, 6, 12, or 24 hours over 1, 2, 3, 4, 5, 6, 7, or 14 days. In other embodiments, the functional half-life is determined by an in vivo assay, wherein levels of a protein encoded by the expression sequence of the circular RNA polynucleotide are measured in patient serum or tissue samples every 1, 2, 6, 12, or 24 hours over 1, 2, 3, 4, 5, 6, 7, or 14 days. In some embodiments, the pre-determined threshold value is the functional half-life of a reference linear RNA polynucleotide comprising the same expression sequence as the circular RNA polynucleotide.

In some embodiments, the circular RNA provided herein may have a higher magnitude of expression than equivalent linear mRNA, e.g., a higher magnitude of expression 24 hours after administration of RNA to cells. In some embodiments, the circular RNA provided herein has a higher magnitude of expression than mRNA comprising the same expression sequence, 5moU modifications, an optimized UTR, a cap, and/or a polyA tail.

In some embodiments, the circular RNA provided herein may be less immunogenic than an equivalent mRNA when exposed to an immune system of an organism or a certain type of immune cell. In some embodiments, the circular RNA provided herein is associated with modulated production of cytokines when exposed to an immune system of an organism or a certain type of immune cell. For example, in some embodiments, the circular RNA provided herein is associated with reduced production of IFN-β1, RIG-I, IL-2, IL-6, IFNγ, and/or TNFα when exposed to an immune system of an organism or a certain type of immune cell as compared to mRNA comprising the same expression sequence. In some embodiments, the circular RNA provided herein is associated with less IFN-β1, RIG-I, IL-2, IL-6, IFNγ, and/or TNFα transcript induction when exposed to an immune system of an organism or a certain type of immune cell as compared to mRNA comprising the same expression sequence. In some embodiments, the circular RNA provided herein is less immunogenic than mRNA comprising the same expression sequence. In some embodiments, the circular RNA provided herein is less immunogenic than mRNA comprising the same expression sequence, modified nucleotides (e.g., 5moU modifications), an optimized UTR, a cap, and/or a polyA tail.

In some embodiments, the circular RNA provided herein can be encapsulated by a transfer vehicle (e.g., LNPs), which can deliver the circular RNA constructs. Encapsulating the circular RNA in the transfer vehicle, for example can efficiently introduce the CAR genes to the T cells. The transfer vehicles can comprise, e.g., ionizable lipids, PEG-modified lipids, helper lipids, and/or structural lipids, that are capable of encapsulating the circular RNAs. Pharmaceutical compositions are provided for circular RNA constructs comprising an IRES, an expression sequence, and a transfer vehicle.

In certain embodiments, the circular RNA constructs provided herein can be transfected into a cell as is or can be transfected in DNA vector form and transcribed in the cell. Transcription of circular RNA from a transfected DNA vector can be via added polymerases or polymerases encoded by nucleic acids transfected into the cell, or preferably via endogenous polymerases. Accordingly, also provided herein is a eukaryotic cell comprising a circular RNA polynucleotide provided herein. In some embodiments, the eukaryotic cell is a human cell. In some embodiments, the eukaryotic cell is an immune cell. In some embodiments, the eukaryotic cell is a T cell, dendritic cell, macrophage, B cell, neutrophil, or basophil. Also provided herein is a prokaryotic cell comprising a circular RNA polynucleotide provided herein.

In some embodiments, provided herein is a T cell, e.g., human T cell, comprising the circular RNA constructs provided herein. In some embodiments, provided herein is a helper T cell, e.g., human helper T cell, comprising the circular RNA constructs provided herein. In some embodiments, provided herein is a cytotoxic T cell, e.g., human cytotoxic T cell, comprising the circular RNA constructs provided herein. In some embodiments, provided herein is a NK cell, e.g., human NK cell, comprising the circular RNA constructs provided herein. In some embodiments, provided herein is a macrophage, e.g., human macrophage, comprising the circular RNA constructs provided herein. In some embodiments, provided herein is a monocyte, e.g., human monocyte, comprising the circular RNA constructs provided herein. In some embodiments, provided herein is a myeloid cell, human monocyte, comprising the circular RNA constructs provided herein. In some embodiments, these cells are present in the bone marrow. In some embodiments, these cells are present in the spleen. In some embodiments, these cells are present in the blood, e.g., peripheral blood.

In some embodiments, provided herein is a CD3+ cell, e.g., human CD3+ cell, comprising the circular RNA constructs provided herein. In some embodiments, provided herein is a CD4+ cell, e.g., human CD4+ cell, comprising the circular RNA constructs provided herein. In some embodiments, provided herein is a CD8+ cell, e.g., human CD8+ cell, comprising the circular RNA constructs provided herein. In some embodiments, provided herein is a CD14+ cell, e.g., human CD14+ cell, comprising the circular RNA constructs provided herein. In some embodiments, provided herein is a CD16+ cell, e.g., human CD16+ cell, comprising the circular RNA constructs provided herein. In some embodiments, provided herein is a CD56+ cell, e.g., human CD56+ cell, comprising the circular RNA constructs provided herein. In some embodiments, provided herein is a CD11B+ cell, e.g., human CD11B+ cell, comprising the circular RNA constructs provided herein. In some embodiments, provided herein is a CD33+ cell, e.g., human CD33+ cell, comprising the circular RNA constructs provided herein. In some embodiments, provided herein is a CD33+CD14+ cell, e.g., human CD33+CD14+ cell, comprising the circular RNA constructs provided herein. In some embodiments, provided herein is a CD33+CD14+ cell, e.g., human CD33+CD64+ cell, comprising the circular RNA constructs provided herein. In some embodiments, these cells are present in the bone marrow. In some embodiments, these cells are present in the spleen. In some embodiments, these cells are present in the blood, e.g., peripheral blood.

The circular RNA can be unmodified, partially modified or completely modified. In one embodiment, the circular RNA contains at least one nucleoside modification. In one embodiment, up to 100% of the nucleosides of the circular RNA are modified. In one embodiment, at least one nucleoside modification is a uridine modification or an adenosine modification. In one embodiment, at least one nucleoside modification is selected from N6-methyladenosine (m6A), pseudouridine (ψ), N1-methylpseudouridine (mlψ), and 5-methoxyuridine (5moU). In one embodiment, the precursor RNA is modified with methylpseudouridine (mlψ).

In certain embodiments, a provided polynucleotide (e.g., a DNA template, a precursor RNA polynucleotide, or a circular RNA polynucleotide) comprises modified nucleotides and/or modified nucleosides. In some embodiments, the modified nucleoside is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine). In other embodiments, the modified nucleoside is m¹A (1-methyladenosine); m2A (2-methyladenosine); Am (2′-O-methyladenosine); ms² m⁶A (2-methylthio-N⁶-methyladenosine); i⁶A (N⁶-isopentenyladenosine); ms²i6A (2-methylthio-N⁶ isopentenyladenosine); i⁶A (N⁶-(cis-hydroxyisopentenyl) adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N6-methyl-Ne-threonylcarbamoyladenosine); hn⁶A (N⁶-hydroxynorvalylcarbamoyladenosine); ms²hn⁶ A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m₂ ²G (N²,N²-dimethylguanosine); m²Gm (N2,2′-O-dimethylguanosine); m² ₂Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G′ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl) uridine); ho⁵U (5-hydroxyuridine); mo⁵U (5-methoxyuridine); cmo⁵U (uridine 5-oxyacetic acid); memo⁵U (uridine 5-oxyacetic acid methyl ester); chm⁵U (5-(carboxyhydroxymethyl) uridine)); mchm⁵U (5-(carboxyhydroxymethyl) uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵S²U (5-aminomethyl-2-thiouridine); mnm⁵U (5-methylaminomethyluridine); mnm⁵s²U (5-methylaminomethyl-2-thiouridine); mnm⁵sc²U (5-methylaminomethyl-2-selenouridine); ncm⁵U (5-carbamoylmethyluridine); ncm⁵Um (5-carbamoylmethyl-2′-O-methyluridine); cmnm³U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶ ₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶ ₂Am (N°,N6,0-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm ⁵U (5-taurinomethyluridine); τm⁵ s²U (5-taurinomethyl-2-thiouridine)); imG-14 (4-demethylwyosine); imG2 (isowyosine); or ac⁶A (N⁶-acetyladenosine).

In some embodiments, the modified nucleoside may include a compound selected from the group of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In another embodiment, the modifications are independently selected from the group consisting of 5-methylcytosine, pseudouridine and 1-methylpseudouridine.

In some embodiments, the modified ribonucleosides include 5-methylcytidine, 5-methoxyuridine, 1-methyl-pseudouridine, N6-methyladenosine, and/or pseudouridine. In some embodiments, such modified nucleosides provide additional stability and resistance to immune activation.

Various circular RNA, circular RNA constructs, compositions comprising circular RNA, precursor RNA, and related methods are described, for example in WO2019236673, WO2020237227, WO2021113777, WO2021226597, WO2021189059, WO2021236855, WO2022261490, WO2023056033, and WO2023081526, which are each incorporated by reference in their entireties.

### A. Enhanced Intron Elements and Enhanced Exon Elements

The circular RNA provided herein can comprise an enhanced intron element or fragment and enhanced exon element or fragment. In certain embodiments, as provided herein, the enhanced intron elements and enhanced exon elements may comprise spacers, duplex regions, affinity sequences, intron fragments, exon fragments and various untranslated elements. These sequences within the enhanced intron elements or enhanced exon elements are arranged to optimize circularization or protein expression.

In certain embodiments, the DNA template, precursor linear RNA polynucleotide and circular RNA provided herein comprise a first (5′) and/or a second (3′) spacer. In some embodiments, the DNA template or precursor linear RNA polynucleotide comprises one or more spacers in the enhanced intron elements. In some embodiments, the DNA template, precursor linear RNA polynucleotide comprises one or more spacers in the enhanced exon elements. In certain embodiments, the DNA template or linear RNA polynucleotide comprises a spacer in the 3′ enhanced intron fragment and a spacer in the 5′ enhanced intron fragment. In certain embodiments, DNA template, precursor linear RNA polynucleotide, or circular RNA comprises a spacer in the 3′ enhanced exon fragment and another spacer in the 5′ enhanced exon fragment to aid with circularization or protein expression due to symmetry created in the overall sequence.

In some embodiments, including a spacer between the 3′ group I intron fragment and the core functional element may conserve secondary structures in those regions by preventing them from interacting, thus increasing splicing efficiency. In some embodiments, the first (between 3′ group I intron fragment and core functional element) and second (between the two expression sequences and core functional element) spacers comprise additional base pairing regions that are predicted to base pair with each other and not to the first and second duplex regions. In other embodiments, the first (between 3′ group I intron fragment and core functional element) and second (between the one of the core functional element and 5′ group I intron fragment) spacers comprise additional base pairing regions that are predicted to base pair with each other and not to the first and second duplex regions. In some embodiments, such spacer base pairing brings the group I intron fragments in close proximity to each other, further increasing splicing efficiency. Additionally, in some embodiments, the combination of base pairing between the first and second duplex regions, and separately, base pairing between the first and second spacers, promotes the formation of a splicing bubble containing the group I intron fragments flanked by adjacent regions of base pairing. Typical spacers are contiguous sequences with one or more of the following qualities: 1) predicted to avoid interfering with proximal structures, for example, the IRES, expression sequence, aptamer, or intron; 2) is at least 7 nt long and no longer than 100 nt; 3) is located after and adjacent to the 3′ intron fragment and/or before and adjacent to the 5′ intron fragment; and 4) contains one or more of the following: a) an unstructured region at least 5 nt long, b) a region of base pairing at least 5 nt long to a distal sequence, including another spacer, and c) a structured region at least 7 nt long limited in scope to the sequence of the spacer. Spacers may have several regions, including an unstructured region, a base pairing region, a hairpin/structured region, and combinations thereof. In an embodiment, the spacer has a structured region with high GC content. In an embodiment, a region within a spacer base pairs with another region within the same spacer. In an embodiment, a region within a spacer base pairs with a region within another spacer. In an embodiment, a spacer comprises one or more hairpin structures. In an embodiment, a spacer comprises one or more hairpin structures with a stem of 4 to 12 nucleotides and a loop of 2 to 10 nucleotides. In an embodiment, there is an additional spacer between the 3′ group I intron fragment and the core functional element. In an embodiment, this additional spacer prevents the structured regions of the IRES or aptamer of a TIE from interfering with the folding of the 3′ group I intron fragment or reduces the extent to which this occurs. In some embodiments, the 5′ spacer sequence is at least 7, 8, 9, 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′ spacer sequence is between 5 and 50, 10 and 50, 20 and 50, 20 and 40, and/or 25 and 35 nucleotides in length. In certain embodiments, the 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 5′ spacer sequence is a polyA sequence. In another embodiment, the 5′ spacer sequence is a polyAC sequence. In one embodiment, a spacer comprises about 10%, 20%, 30%, 40%, 50%, 60%, 70%, 80%, 90%, or 100% polyAC content. In one embodiment, a spacer comprises about 10%, 20%, 30%, 40%, 50%, 60%, 70%, 80%, 90%, or 100% polypyrimidine (C/T or C/U) content.

In some embodiments, the DNA template and precursor linear RNA polynucleotides and circular RNA polynucleotide provided herein comprise a first (5′) duplex region and a second (3′) duplex region. In certain embodiments, the DNA template and precursor linear RNA polynucleotide comprises a 5′ external duplex region located within the 3′ enhanced intron fragment and a 3′ external duplex region located within the 5′ enhanced intron fragment. In some embodiments, the DNA template, precursor linear RNA polynucleotide and circular RNA polynucleotide comprise a 5′ internal duplex region located within the 3′ enhanced exon fragment and a 3′ internal duplex region located within the 5′ enhanced exon fragment. In some embodiments, the DNA polynucleotide and precursor linear RNA polynucleotide comprises a 5′ external duplex region, 5′ internal duplex region, a 3′ internal duplex region, and a 3′ external duplex region.

In certain embodiments, the first and second duplex regions may form perfect or imperfect duplexes. Thus, in certain embodiments at least 75%, 80%, 85%, 90%, 91%, 92%, 93%, 94%, 95%, 96%, 97%, 98%, 99% or 100% of the first and second duplex regions may be base paired with one another. In some embodiments, the duplex regions are predicted to have less than 50% (e.g., less than 45%, less than 40%, less than 35%, less than 30%, less than 25%) base pairing with unintended sequences in the RNA (e.g., non-duplex region sequences). In some embodiments, including such duplex regions on the ends of the precursor RNA strand, and adjacent or very close to the group I intron fragment, bring the group I intron fragments in close proximity to each other, increasing splicing efficiency. In some embodiments, the duplex regions are 3 to 100 nucleotides in length (e.g., 3-75 nucleotides in length, 3-50 nucleotides in length, 20-50 nucleotides in length, 35-50 nucleotides in length, 5-25 nucleotides in length, 9-19 nucleotides in length). In some embodiments, the duplex regions are about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In some embodiments, the duplex regions have a length of about 9 to about 50 nucleotides. In one embodiment, the duplex regions have a length of about 9 to about 19 nucleotides. In some embodiments, the duplex regions have a length of about 20 to about 40 nucleotides. In certain embodiments, the duplex regions have a length of about 30 nucleotides.

In other embodiments, the DNA template, precursor linear RNA polynucleotide, or circular RNA polynucleotide does not comprise of any duplex regions to optimize translation or circularization.

In certain embodiments, as provided herein, the DNA template or precursor linear RNA polynucleotide may comprise an affinity tag. In some embodiments, the affinity tag is located in the 3′ enhanced intron element. In some embodiments, the affinity tag is located in the 5′ enhanced intron element. In some embodiments, both (3′ and 5′) enhanced intron elements each comprise an affinity tag. In one embodiment, an affinity tag of the 3′ enhanced intron element is the length as an affinity tag in the 5′ enhanced intron element. In some embodiments, an affinity tag of the 3′ enhanced intron element is the same sequence as an affinity tag in the 5′ enhanced intron element. In some embodiments, the affinity sequence is placed to optimize oligo-dT purification.

In some embodiments, the one or more affinity tags present in a precursor linear RNA polynucleotide are removed upon circularization. In some embodiments, affinity tags are added to remaining linear RNA after circularization of RNA is performed. In some such embodiments, the affinity tags are added enzymatically to linear RNA. The presence of one or more affinity tags in linear RNA and their absence from circular RNA can facilitate purification of circular RNA. In some embodiments, such purification is performed using a negative selection or affinity-purification method. In some embodiments, such purification is performed using a binding agent that preferentially or specifically binds to the affinity tag.

In some embodiments, an affinity tag comprises a polyA region. In some embodiments the polyA region is at least 15, 30, or 60 nucleotides long. In some embodiments, the affinity tag comprising a polyA region is present in two places in a precursor linear RNA. In some embodiments, one or both polyA regions is 15-50 nucleotides long. In some embodiments, one or both polyA regions is 20-25 nucleotides long. The polyA sequence(s) is removed upon circularization. Thus, an oligonucleotide hybridizing with the polyA sequence, such as a deoxythymidine oligonucleotide (oligo (dT)) conjugated to a solid surface (e.g., a resin), can be used to separate circular RNA from its precursor RNA.

In some embodiments, an affinity tag comprises a sequence that is absent from the circular RNA product. In some such embodiments, the sequence that is absent from the circular RNA product is a dedicated binding site (DBS). In some embodiments, the DBS is an unstructured sequence, i.e., a sequence that does not form a defined structural element, such as a hairpin loop, contiguous dsRNA region, or triple helix. In some embodiments, the DBS sequence forms a random coil. In some embodiments, the DBS comprises at least 25% GC content, at least 50% GC content, at least 75% GC content, or at least 100% GC content. In some embodiments, the DBS comprises at least 25% AC content, at least 50% AC content, at least 75% AC content, or 100% AC content. In some embodiments, the DBS is at least 15, 30, or 60 nucleotides long. In some embodiments, the affinity tag comprising a DBS is present in two places in a precursor linear RNA. In some embodiments, the DBS sequences are each independently 15-50 nucleotides long. In some embodiments, the DBS sequences are each independently 20-25 nucleotides long.

In some embodiments, the DBS sequence(s) is removed upon circularization. Thus, binding agents comprising oligonucleotides comprising a sequence that is complementary to the DBS can be used to facilitate purification of circular RNA. For example, the binding agent may comprise an oligonucleotide complementary to a DBS conjugated to a solid surface (e.g., a resin).

In some embodiments, an affinity sequence or other type of affinity handle, such as biotin, is added to linear RNA by ligation. In some embodiments, an oligonucleotide comprising an affinity sequence is ligated to the linear RNA. In some embodiments, an oligonucleotide conjugated to an affinity handle is ligated to the linear RNA. In some embodiments, a solution comprising the linear RNA ligated to the affinity sequence or handle and the circular RNA that does not comprise an affinity sequence or handle are contacted with a binding agent comprising a solid support conjugated to an oligonucleotide complementary to the affinity sequence or to a binding partner of the affinity handle, such that the linear RNA binds to the binding agent, and the circular RNA is eluted or separated from the solid support.

Any purification method for circular RNA described herein may comprise one or more buffer exchange steps. In some embodiments, buffer exchange is performed after in vitro transcription (IVT) and before additional purification steps. In some such embodiments, the IVT reaction solution is buffer exchanged into a buffer comprising Tris. In some embodiments, the IVT reaction solution is buffer exchanged into a buffer comprising greater than 1 mM or greater than 10 mM one or more monovalent salts, such as NaCl or KCl, and optionally comprising EDTA. In some embodiments, buffer exchange is performed after purification of circular RNA is complete. In some embodiments, buffer exchange is performed after IVT and after purification of circular RNA. In some embodiments, the buffer exchange that is performed after purification of circular RNA comprises exchange of the circular RNA into water or storage buffer. In some embodiments, the storage buffer comprises 1 mM sodium citrate, pH 6.5.

In certain embodiments, the 3′ enhanced intron element comprises a leading untranslated sequence. In some embodiments, the leading untranslated sequence is a the 5′ end of the 3′ enhanced intron fragment. In some embodiments, the leading untranslated sequence comprises of the last nucleotide of a transcription start site (TSS). In some embodiments, the TSS is chosen from a viral, bacterial, or eukaryotic DNA template. In one embodiment, the leading untranslated sequence comprise the last nucleotide of a TSS and 0 to 100 additional nucleotides. In some embodiments, the TSS is a terminal spacer. In one embodiment, the leading untranslated sequence contains a guanosine at the 5′ end upon translation of an RNA T7 polymerase.

In certain embodiments, the 5′ enhanced intron element comprises a trailing untranslated sequence. In some embodiments, the 5′ trailing untranslated sequence is located at the 3′ end of the 5′ enhanced intron element. In some embodiments, the trailing untranslated sequence is a partial restriction digest sequence. In one embodiment, the trailing untranslated sequence is in whole or in part a restriction digest site used to linearize the DNA template. In some embodiments, the restriction digest site is in whole or in part from a natural viral, bacterial or eukaryotic DNA template. In some embodiments, the trailing untranslated sequence is a terminal restriction site fragment.

### 1. Enhanced Intron Fragments

In certain embodiments, as provided herein, the 3′ enhanced intron element and 5′ enhanced intron element each comprise an intron fragment. In certain embodiments, a 3′ intron fragment is a contiguous sequence at least 75% homologous (e.g., at least 80%, 85%, 90%, 91%, 92%, 93%, 94%, 95%, 96%, 97%, 98%, 99% or 100% homologous) to a 3′ proximal fragment of a natural group I intron including the 3′ splice site dinucleotide. Typically, a 5′ intron fragment is a contiguous sequence at least 75% homologous (e.g., at least 80%, 85%, 90%, 91%, 92%, 93%, 94%, 95%, 96%, 97%, 98%, 99% or 100% homologous) to a 5′ proximal fragment of a natural group I intron including the 5′ splice site dinucleotide. In some embodiments, the 3′ intron fragment includes the first nucleotide of a 3′ group I splice site dinucleotide. In some embodiments, the 5′ intron fragment includes the first nucleotide of a 5′ group I splice site dinucleotide. In other embodiments, the 3′ intron fragment includes the first and second nucleotides of a 3′ group I intron fragment splice site dinucleotide; and the 5′ intron fragment includes the first and second nucleotides of a 3′ group I intron fragment dinucleotide.

### 2. Enhanced Exon Fragments

In certain embodiments, as provided herein, the DNA template, linear precursor RNA polynucleotide, and circular RNA polynucleotide each comprise an enhanced exon fragment. In some embodiments, following a 5′ to 3′ order, the 3′ enhanced exon element is located upstream to core functional element. In some embodiments, following a 5′ to 3′ order, the 5′ enhanced intron element is located downstream to the core functional element.

According to the present disclosure, the 3′ enhanced exon element and 5′ enhanced exon element each comprise an exon fragment. In some embodiments, the 3′ enhanced exon element comprises a 3′ exon fragment. In some embodiments, the 5′ enhanced exon element comprises a 5′ exon fragment. In certain embodiments, as provided herein, the 3′ exon fragment and 5′ exon fragment each comprises a group I intron fragment and 1 to 100 nucleotides of an exon sequence. In certain embodiments, a 3′ intron fragment is a contiguous sequence at least 75% homologous (e.g., at least 80%, 85%, 90%, 91%, 92%, 93%, 94%, 95%, 96%, 97%, 98%, 99% or 100% homologous) to a 3′ proximal fragment of a natural group I intron including the 3′ splice site dinucleotide. Typically, a 5′ group I intron fragment is a contiguous sequence at least 75% homologous (e.g., at least 80%, 85%, 90%, 91%, 92%, 93%, 94%, 95%, 96%, 97%, 98%, 99% or 100% homologous) to a 5′ proximal fragment of a natural group I intron including the 5′ splice site dinucleotide. In some embodiments, the 3′ exon fragment comprises a second nucleotide of a 3′ group I intron splice site dinucleotide and 1 to 100 nucleotides of an exon sequence. In some embodiments, the 5′ exon fragment comprises the first nucleotide of a 5′ group I intron splice site dinucleotide and 1 to 100 nucleotides of an exon sequence. In some embodiments, the exon sequence comprises in part or in whole from a naturally occurring exon sequence from a virus, bacterium or eukaryotic DNA vector. In other embodiments, the exon sequence further comprises a synthetic, genetically modified (e.g., containing modified nucleotide), or other engineered exon sequence.

In one embodiment, where the 3′ intron fragment comprises both nucleotides of a 3′ group I splice site dinucleotide and the 5′ intron fragment comprises both nucleotides of a 5′ group I splice site dinucleotide, the exon fragments located within the 5′ enhanced exon element and 3′ enhanced exon element does not comprise of a group I splice site dinucleotide.

### 3. Exemplary Permutation of the Enhanced Intron Elements & Enhanced Exon Elements

For means of example and not intended to be limiting, in some embodiment, a 3′ enhanced intron element comprises in the following 5′ to 3′ order: a leading untranslated sequence, a 5′ affinity tag, an optional 5′ external duplex region, a 5′ external spacer, and a 3′ intron fragment. In the same embodiments, the 3′ enhanced exon element comprises in the following 5′ to 3′ order: a 3′ exon fragment, an optional 5′ internal duplex region, an optional 5′ internal duplex region, and a 5′ internal spacer. In the same embodiments, the 5′ enhanced exon element comprises in the following 5′ to 3′ order: a 3′ internal spacer, an optional 3′ internal duplex region, and a 5′ exon fragment. In still the same embodiments, the 3′ enhanced intron element comprises in the following 5′ to 3′ order: a 5′ intron fragment, a 3′ external spacer, an optional 3′ external duplex region, a 3′ affinity tag, and a trailing untranslated sequence.

### B. Core Functional Element-IRES

In some embodiments, the DNA template, linear precursor RNA polynucleotide, and circular RNA polynucleotide comprise a core functional element. In some embodiments, the core functional element comprises a coding and/or noncoding element. In some embodiments, the core functional element further comprises a translation initiation element (TIE) upstream to the coding or noncoding element, and/or a termination element.

In some embodiments, the core functional element comprises a termination element. In some embodiments, the termination sequence comprises a stop codon. In one embodiment, the termination sequence comprises a stop cassette. In some embodiments, the stop cassette comprises at least 2 stop codons. In some embodiments, the stop cassette comprises at least 2 frames of stop codons. In the same embodiment, the frames of the stop codons in a stop cassette each comprise 1, 2 or more stop codons. In some embodiments, the stop cassette comprises a LoxP or a RoxStopRox, or fit-flanked stop cassette. In the same embodiment, the stop cassette comprises a lox-stop-lox stop cassette.

In some embodiments, the polynucleotides herein comprise a coding or noncoding element or a combination of both. In some embodiments, the coding element comprises an expression sequence. In some embodiments, the coding element encodes at least one therapeutic protein. In some embodiments, the circular RNA encodes two or more polypeptides.

In some embodiments, the core functional element comprises at least one translation initiation element (TIE). TIEs are designed to allow translation efficiency of an encoded protein. In some embodiments, core functional elements comprising one or more coding elements will further comprise one or more TIEs. In some embodiments, a translation initiation element (TIE) comprises a synthetic TIE. In some embodiments, a synthetic TIE comprises aptamer complexes, synthetic IRES or other engineered TIES capable of initiating translation of a linear RNA or circular RNA polynucleotide.

In some embodiments, a TIE comprises an untranslated region (UTR) or a fragment thereof, an aptamer complex or a fragment thereof, or a combination thereof. In certain embodiments, the TIE contains modified nucleotides. In certain embodiments, the TIE provided herein comprise an internal ribosome entry site (IRES). In certain embodiments, the IRES comprises one or more modified nucleotides compared to the wile-type viral IRES or eukaryotic IRES. See, e.g., WO2022/261490, which is incorporated herein by reference in its entirety.

Since the discovery of viral IRESes, there have been difficulties in their classification due to their dissimilarity. It has been observed that there is no common mechanism for functioning of all IRESes. Additionally, no particular structure element has been found that is shared by all IRESes; their sequences lack significant homology. See Nikonov, Biochemistry (Moscow), 2017, Vol. 82, No. 13, pp. 1615-1631. According to one author, four IRES classes have been defined. Type I and II IRESes are found in picornaviruses and can be around 400-500nt long. Type III IRESes concern the Flaviviridae (including HCV) and HCV-like picornaviruses and are characterized by the presence of a pseudoknot upstream from the AUG codon and by the requirement of the first 30nt of the coding sequence. Type IV IRESes are intergenic region (IGR) IRESes, originally identified in cricket paralysis virus (CrPV), which can function in the absence of any start codon and where translation starts at a GCU triplet. See Godet, Int. J. Mol. Sci. 2019, 20, 924; doi: 10.3390/ijms20040924.

Inclusion of an IRES permits the translation of one or more open reading frames from a circular RNA (e.g., open reading frames that form the expression sequences). The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20:102-110; Kobayashi et al., BioTechniques (1996) 21:399-402; and Mosser et al., BioTechniques 1997 22 150-161. In some embodiments, the IRES is capable of facilitating expression of a protein encoded by the precursor RNA in a cell. In some embodiments, the IRES is capable of facilitating expression of the protein, such that the expression level of the protein is comparable to or higher than when a control IRES is used.

A multitude of IRES sequences are available and include sequences derived from a wide variety of viruses, such as from leader sequences of picornaviruses such as the encephalomyocarditis virus (EMCV) UTR (Jang et al., J. Virol. (1989) 63:1651-1660), the polio leader sequence, the hepatitis A virus leader, the hepatitis C virus IRES, human rhinovirus type 2 IRES (Dobrikova et al., Proc. Natl. Acad. Sci. (2003) 100 (25): 15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279 (5): 3389-3397), and the like. Different IRES sequences have varying ability to drive protein expression, and the ability of any particular identified or predicted IRES sequence to drive protein expression from linear mRNA or circular RNA constructs is unknown and unpredictable. In certain embodiments, potential IRES sequences can be bioinformatically identified based on sequence positions in viral sequences. However, the activity of such sequences has been previously uncharacterized. As demonstrated herein, such IRES sequences may have differing protein expression capability depending on cell type, for example in T cells, liver cells, or muscle cells. In some embodiments, the novel IRES sequences described herein may have at least 2, 3, 4, 5, 6, 7, 8, 9, 10, 20, 50, or 100 fold increased expression in a particular cell type compared to previously described EMCV IRES sequences.

In some embodiments, the IRES is an Aalivirus, Ailurivirus, Ampivirus, Anativirus, Aphthovirus, Aquamavirus, Avihepatovirus, Avisivirus, Boosepivirus, Bopivirus, Caccilivirus, Cardiovirus, Cosavirus, Crahelivirus, Crohivirus, Danipivirus, Dicipivirus, Diresapivirus, Enterovirus, Erbovirus, Felipivirus, Fipivirus, Gallivirus, Gruhelivirus, Grusopivirus, Harkavirus, Hemipivirus, Hepatovirus, Hunnivirus, Kobuvirus, Kunsagivirus, Limnipivirus, Livupivirus, Ludopivirus, Malagasivirus, Marsupivirus, Megrivirus, Mischivirus, Mosavirus, Mupivirus, Myrropivirus, Orivirus, Oscivirus, Parabovirus, Parechovirus, Pasivirus, Passerivirus, Pemapivirus, Poecivirus, Potamipivirus, Pygoscepivirus, Rabovirus, Rafivirus, Rajidapivirus, Rohelivirus, Rosavirus, Sakobuvirus, Salivirus, Sapelovirus, Senecavirus, Shanbavirus, Sicinivirus, Symapivirus, Teschovirus, Torchivirus, Tottorivirus, Tremovirus, Tropivirus, Hepacivirus, Pegivirus, Pestivirus, Flavivirus IRES. In some embodiments herein, the IRES is selected from an Enterovirus, Kobuvirus, Parechovirus, Hunnivirus, Passerivirus, Mischivirus, and Cardiovirus.

In some embodiments, the IRES is an IRES sequence of Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Himetobi P virus, Hepatitis C virus, Hepatitis A virus, Hepatitis GB virus, Foot and mouth disease virus, Human enterovirus 71, Equine rhinitis virus, Ectropis obliqua picoma-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPAI, Human AMLI/RUNX1, Drosophila antennapedia, Human AQP4, Human ATIR, Human BAG-1, Human BCL2, Human BiP, Human c-IAPI, Human c-myc, Human eIF4G, Mouse NDST4L, Human LEF1, Mouse HIFI alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAPI, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SHI, Salivirus FHB, Salivirus NG-JI, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Salivirus A BN5, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24 or an aptamer to eIF4G.

In some embodiments, the IRES comprises in whole or in part a eukaryotic or cellular IRES. In certain embodiments, the IRES is from a human gene, where the human gene is ABCF1, ABCG1, ACAD10, ACOT7, ACSS3, ACTG2, ADCYAP1, ADK, AGTR1, AHCYL2, AHI1, AKAP8L, AKRIA1, ALDH3A1, ALDOA, ALG13, AMMECR1L, ANGPTL4, ANK3, AOC3, AP4B1, AP4E1, APAF1, APBB1, APC, APH1A, APOBEC3D, APOM, APP, AQP4, ARHGAP36, ARL13B, ARMC8, ARMCX6, ARPC1A, ARPC2, ARRDC3, ASAPI, ASB3, ASB5, ASCL1, ASMTL, ATF2, ATF3, ATG4A, ATP5B, ATP6V0A1, ATXN3, AURKA, AURKA, AURKA, AURKA, B3GALNT1, B3GNTL1, B4GALT3, BAAT, BAG1, BAIAP2, BAIAP2L2, BAZ2A, BBX, BCAR1, BCL2, BCSIL, BET1, BID, BIRC2, BPGM, BPIFA2, BRINP2, BSG, BTN3A2, C12orf43, C14orf93, C17orf62, Clorf226, C21orf62, C2orf15, C4BPB, C4orf22, C9orf84, CACNAIA, CALCOCO2, CAPN11, CASP12, CASP8AP2, CAVI, CBX5, CCDCl20, CCDCl7, CCDCl86, CCDC51, CCNI, CCND1, CCNTI, CD2BP2, CD9, CDC25C, CDC42, CDC7, CDCA7L, CDIPI, CDKI, CDKIIA, CDKNIB, CEACAM7, CEP295NL, CFLAR, CHCHD7, CHIA, CHICI, CHMP2A, CHRNA2, CLCN3, CLEC12A, CLEC7A, CLECL1, CLRN1, CMSS1, CNIH1, CNR1, CNTN5, COG4, COMMD1, COMMD5, CPEB1, CPS1, CRACR2B, CRBN, CREM, CRYBG1, CSDE1, CSF2RA, CSNK2A1, CSTF3, CTCFL, CTH, CTNNA3, CINNB1, CTNNB1, CTNND1, CTSL, CUTA, CXCR5, CYB5R3, CYP24A1, CYP3A5, DAGI, DAP3, DAP5, DAXX, DCAF4, DCAF7, DCLRE1A, DCP1A, DCTN1, DCTN2, DDX19B, DDX46, DEFB123, DGKA, DGKD, DHRS4, DHX15, DIO3, DLG1, DLL4, DMD UTR, DMD ex5, DMKN, DNAH6, DNAL4, DUSP13, DUSP19, DYNCII2, DYNLRB2, DYRKIA, ECI2, ECT2, EIFIAD, EIF2B4, EIF4G1, EIF4G2, EIF4G3, ELANE, ELOVL6, ELP5, EMCN, ENO1, EPB41, ERMN, ERVV-1, ESRRG, ETFB, ETFBKMT, ETV1, ETV4, EXD1, EXT1, EZH2, FAM111B, FAM157A, FAM213A, FBX025, FBX09, FBXW7, FCMR, FGF1, FGF1, FGFIA, FGF2, FGF2, FGF-9, FHL5, FMRI, FN1, FOXP1, FTHI, FUBP1, G3BP1, GABBRI, GALC, GART, GAS7, gastrin, GATA1, GATA4, GFM2, GHR, GJB2, GLI1, GLRA2, GMNN, GPAT3, GPATCH3, GPR137, GPR34, GPR55, GPR89A, GPRASP1, GRAP2, GSDMB, GSTO2, GTF2B, GTF2H4, GUCY1B2, HAX1, HCST, HIGD1A, HIGD1B, HIPK1, HISTIH1C, HISTIH3H, HK1, HLA-DRB4, HMBS, HMGA1, HNRNPC, HOPX, HOXA2, HOXA3, HPCAL1, HR, HSP90AB1, HSPA1A, HSPA4L, HSPA5, HYPK, IFFO1, IFT74, IFT81, IGF1, IGF1R, IGF1R, IGF2, IL11, IL17RE, ILIRL1, IL1RN, IL32, IL6, ILF2, ILVBL, INSR, INTS13, IP6K1, ITGA4, ITGAE, KCNE4, KERA, KIAA0355, KIAA0895L, KIAA1324, KIAA1522, KIAA1683, KIF2C, KIZ, KLHL31, KLK7, KRRI, KRT14, KRT17, KRT33A, KRT6A, KRTAP10-2, KRTAP13-3, KRTAP13-4, KRTAP5-11, KRTCAP2, LACRT, LAMBI, LAMB3, LANCLI, LBX2, LCAT, LDHA, LDHAL6A, LEF1, LINC-PINT, LMO3, LRRC4C, LRRC7, LRTOMT, LSM5, LTB4R, LYRM1, LYRM2, MAGEA1I, MAGEA8, MAGEBI, MAGEB16, MAGEB3, MAPT, MARS, MCIR, MCCCI, METTL12, METTL7A, MGC16025, MGC16025, MIA2, MIA2, MITF, MKLN1, MNT, MORF4L2, MPD6, MRFAP1, MRPL21, MRPS12, MSI2, MSLN, MSN, MT2A, MTFRIL, MTMR2, MTRR, MTUSI, MYB, MYC, MYCL, MYCN, MYL10, MYL3, MYLK, MYOIA, MYT2, MZBI, NAPILI, NAVI, NBAS, NCF2, NDRGI, NDST2, NDUFA7, NDUFB11, NDUFC1, NDUFS1, NEDD4L, NFAT5, NFE2L2, NFE2L2, NFIA, NHEJ1, NHP2, NITI, NKRF, NME1-NME2, NPAT, NR3C1, NRBF2, NRF1, NTRK2, NUDCD1, NXF2, NXT2, ODCI, ODF2, OPTN, OR10R2, OR1IL1, OR2M2, OR2M3, OR2M5, OR2T10, OR4C15, OR4F17, OR4F5, OR5H1, OR5K1, OR6C3, OR6C75, OR6N1, OR7G2, p53, P2RY4, PAN2, PAQR6, PARP4, PARP9, PC, PCBP4, PCDHGC3, PCLAF, PDGFB, PDZRN4, PELO, PEMT, PEX2, PFKM, PGBD4, PGLYRP3, PHLDA2, PHTFI, PI4 KB, PIGC, P1M1, PKD2L1, PKM, PLCB4, PLD3, PLEKHAI, PLEKHBI, PLS3, PML, PNMA5, PNN, POCIA, POCIB, POLD2, POLD4, POU5F1, PPIG, PQBPI, PRAME, PRPF4, PRRII, PRRTI, PRSS8, PSMA2, PSMA3, PSMA4, PSMD11, PSMD4, PSMD6, PSME3, PSMG3, PTBP3, PTCHI, PTHLH, PTPRD, PUS7L, PVRIG, QPRT, RAB27A, RAB7B, RABGGTB, RAETIE, RALGDS, RALYL, RARB, RCVRN, REG3G, RFC5, RGL4, RGS19, RGS3, RHD, RINL, RIPOR2, RITA1, RMDN2, RNASEI, RNASE4, RNF4, RPA2, RPL17, RPL21, RPL26L1, RPL28, RPL29, RPL41, RPL9, RPS11, RPS13, RPS14, RRBP1, RSUI, RTP2, RUNX1, RUNXITI, RUNXITI, RUNX2, RUSCI, RXRG, S100A13, S100A4, SATI, SCHIP1, SCMHI, SEC14L1, SEMA4A, SERPINA1, SERPINB4, SERTAD3, SFTPD, SH3D19, SHC1, SHMTI, SHPRH, SIMI, SIRT5, SLC11A2, SLC12A4, SLC16A1, SLC25A3, SLC26A9, SLC5A11, SLC6A12, SLC6A19, SLC7A1, SLFNII, SLIRP, SMAD5, SMARCADI, SMNI, SNCA, SNRNP200, SNRPB2, SNX12, SODI, SOX13, SOX5, SP8, SPARCLI, SPATA12, SPATA31C2, SPN, SPOP, SQSTMI, SRBDI, SRC, SREBFI, SRPK2, SSB, SSB, SSBP1, ST3GAL6, STABI, STAMBP, STAUI, STAUI, STAUI, STAUI, STAUI, STK16, STK24, STK38, STMNI, STX7, SULT2B1, SYK, SYNPR, TAFIC, TAGLN, TANK, TAS2R40, TBCID15, TBXASI, TCF4, TDGF1, TDP2, TDRD3, TDRD5, TESK2, THAP6, THBD, THTPA, TIAM2, TKFC, TKTLI, TLR10, TM9SF2, TMC6, TMC02, TMED10, TMEM116, TMEM126A, TMEM159, TMEM208, TMEM230, TMEM67, TMPRSS13, TMUB2, TNFSF4, TNIP3, TP53, TP53, TP73, TRAFI, TRAKI, TRIM31, TRIM6, TRMT1, TRMT2B, TRPM7, TRPM8, TSPEAR, TTC39B, TTLL11, TUBB6, TXLNB, TXNIP, TXNLI, TXNRDI, TYROBP, U2AFI, UBAI, UBE2D3, UBE2I, UBE2L3, UBE2V1, UBE2V2, UMPS, UNG, UPP2, USMG5, USP18, UTP14A, UTRN, UTS2, VDR, VEGFA, VEGFA, VEPHI, VIPAS39, VPS29, VSIGIOL, WDHDI, WDR12, WDR4, WDR45, WDYHVI, WRAP53, XIAP, XPNPEP3, YAPI, YWHAZ, YYIAPI, ZBTB32, ZNF146, ZNF250, ZNF385A, ZNF408, ZNF410, ZNF423, ZNF43, ZNF502, ZNF512, ZNF513, ZNF580, ZNF609, ZNF707, or ZNRD1.

In some embodiments, the cell is a myotube. In some embodiments, the IRES is derived from Bopivirus, Oscivirus, Hunnivirus, Passerivirus, Mischivirus, Kobuvirus, Enterovirus, Cardiovirus, Salivirus, Rabovirus, Parechovirus, Gallivirus, or Sicinivirus. In some embodiments, the IRES is derived from Hunnivirus, Passerivirus, Kobuvirus, Bopivirus, or Enterovirus. In some embodiments, the IRES is derived from Enterovirus I, Enterovirus F, Enterovirus E, Enterovirus J, Enterovirus C, Enterovirus A, Enterovirus B, Aichivirus B, Parechovirus A, Cardiovirus F, Cardiovirus B, or Cardiovirus E.

In some embodiments, the cell is a hepatocyte. In some embodiments, the IRES is derived from Enterovirus, Bopivirus, Mischivirus, Gallivirus, Oscivirus, Cardiovirus, Kobuvirus, Rabovirus, Salivirus, Parechovirus, Hunnivirus, Tottorivirus, Passerivirus, Cosavirus, or Sicinivirus. In some embodiments, the IRES is derived from Enterovirus, Mischivirus, Kobuvirus, Bopivirus, or Gallivirus. In some embodiments, the IRES is derived from Enterovirus B, Enterovirus A, Enterovirus D, Enterovirus J, Enterovirus C, Rhinovirus B, Enterovirus H, Enterovirus I, Enterovirus E, Enterovirus F, Aichivirus B, Aichivirus A, Parechovirus A, Cardiovirus F, Cardiovirus E, or Cardiovirus B.

In some embodiments, the cell is a T cell. In some embodiments, the IRES is derived from Passerivirus, Bopivirus, Hunnivirus, Mischivirus, Enterovirus, Kobuvirus, Rabovirus, Tottorivirus, Salivirus, Cardiovirus, Parechovirus, Megrivirus, Allexivirus, Oscivirus, or Shanbavirus. In some embodiments, the IRES is derived from Passerivirus, Hunnivirus, Mischivirus, Enterovirus, or Kobuvirus. In some embodiments, the IRES is derived from Enterovirus I, Enterovirus D, Enterovirus C, Enterovirus A, Enterovirus J, Enterovirus H, Aichivirus B, Parechovirus A, or Cardiovirus B.

For driving protein expression, the circular RNA comprises an IRES operably linked to a protein coding sequence. Exemplary IRES sequences are provided in Table 1A. In some embodiments, the circular RNA constructs and related pharmaceutical compositions disclosed herein comprise an IRES sequence that is at least 80%, 85%, 90%, 91%, 92%, 93%, 94%, 95%, 96%, 97%, 98%, or 99% identical to an IRES sequence in Table 1A or an IRES from a construct of SEQ ID NOs: 50-61 or any of Constructs A-P of Table IB. In some embodiments, the circular RNA constructs and related pharmaceutical compositions disclosed herein comprise an IRES sequence in Table 1A or an IRES from a construct of SEQ ID NOs: 50-61 or any of Constructs A-P of Table 1B. Modifications of IRES and accessory sequences are disclosed herein to increase or reduce IRES activities, for example, by truncating the 5′ and/or 3′ ends of the IRES, adding a spacer 5′ to the IRES, modifying the 6 nucleotides 5′ to the translation initiation site (Kozak sequence), modification of alternative translation initiation sites, and creating chimeric/hybrid IRES sequences. In some embodiments, the IRES sequence in the circular RNA constructs and related pharmaceutical compositions disclosed herein comprises one or more of these modifications relative to a native IRES.

In particular embodiments, the circular RNA constructs disclosed herein comprise an IRES and at least one expression sequence encoding a binding molecule. In particular embodiments, the IRES sequences are the exemplary IRES sequences provided in Table 1A, below, or an IRES from a construct of SEQ ID NOs: 50-61 or any of Constructs A-P of Table 1B. In some embodiments, the circular RNA constructs and related pharmaceutical compositions disclosed herein comprise an IRES sequence that is at least 80%, 85%, 90%, 91%, 92%, 93%, 94%, 95%, 96%, 97%, 98%, or 99% identical to an IRES sequence in Table IA or an IRES from a construct of SEQ ID NOs: 50-61 or any of Constructs A-P of Table 1B. In some embodiments, the circular RNA constructs and related pharmaceutical compositions disclosed herein comprise an IRES sequence in Table 1A or an IRES from a construct of SEQ ID NOs: 50-61 or any of Constructs A-P of Table 1B and at least one expression sequence encoding a binding molecule.

TABLE 1A
IRES Sequences

| IRES | SEQ | |
|---|---|---|
| NO: | ID NO: | Sequence |
| 1-1 | 1 | TTAAAACAGCTCTGGGGTTGTTCCCACCCCAGAGGCCCACGTGGCGGCCAGTACTCC |
| | | GGTATTACGGTACCCTTGTACGCCTGTTTTATACTCCCTTCCCCTGTAACTTAGAAGC |
| | | ATACAAACCAAGTTCAATAGAAGGGGGTACAAACCAGTACCACCACGAACAAGCAC |
| | | TCCTGTTTCCCCGGTGACATTGCATAGACTGTACCCACGGTTGAAAGCGATCGATCC |
| | | GTTACCCGCTCCTGTACTTCGAGAAGCCTAGTATCATCTTGGAATCTTCGATGCGTTG |
| | | CGCTCAGCACTCAACCCCAGAGTGTAGCTTAGGCTGATGAGTCTGGACGTCCCCCAC |
| | | CGGCGACGGTGGTCCAGGCTGCGTTGGCGGCCTACCTGTGGCCCAAAGCCACAGGAC |
| | | GCTAGTTGTGAACAAGGTGTGAAGAGCCTATTGAGCTACAAGAGAGTCCTCCGGCCC |
| | | CTGAATGCGGCTAATCCTAACCACGGAGCAGGCAGTTGCAAACCAGCAACCGGCCT |
| | | GTCGTAACGCGCAAGTCTGTGGCGGAACCGACTACTTTGGGTGTCCGTGTTTCCTTTT |
| | | ATTTTTACAATGGCTGCTTATGGTGACAATCATAGATTGTTATCATAAAGCGACTTGG |
| | | ATTGGCCATCCGGTGAAAGTAAAACACATTGTTTACTTGTTTGTTGGATTCACTCCAA |
| | | TTAACACTTTTACTTACAAACTCATTACAACAACTCTATTAATTAGAGATAAGCATCA |
| | | CA |
| 1-2 | 2 | TTTCCCCTGTTCGTAACTAAGTGTGTGCCCAATCTCCTCACTCCTGCTGGCTTCACCG |
| | | ACCGGCAGTGTCCAAAATGCTAGGTGAATCCCCTCCCTTTCCTCTGGGCTTCTGCCCA |
| | | GCTTCCTCCCCCCAGCCTGACGTGACACAGGCTGTGCAAAGACCCCGCGAAAGCTGC |
| | | CAAAAGTGGCAATTGTGGGTCCCCCCTTTGTAAAGGCGTCGAGTCTTTCTCCCTCAAG |
| | | GCTAGACCCGTCAGTGAATTCTGTCGGGCAACTAGTGACGCCACTGCACGCCTCTGA |
| | | CCTCGGCCGCGGAGTGCTGCCCCCCAAGTCGTGCCCCTGACCACAAGTTGTGCTGTC |
| | | TGGCAAACATTGTCTGTGAGAATGTTCCGCTGTGGCTGCCAAGCCTGGCAACAGGCT |
| | | GCCCCAGTGTGCGTAGTTCTCATCCAGACTTCGGTCTGGCAACTTGCTGTTAAGACAC |
| | | GGCGTAAGGGGCGTGTGCCAACGCCCTGGAACGAGTGTCCACTCTAATACCCCGAGG |
| | | AATGCTACGCAGGTACCCCTGGTTCGCCAGGGATCTGAGCGTAGGCTAATTGTCTAA |
| | | GGGTATTTTCATTTCCCATTCTTTCTTTCTTGTTCATA |
| 1-3 | 3 | TCCCCGGCATGAGAGGAATAGACTCTTTCAGGGTTGAAGCCACGAGTGTCGTTACCC |
| | | GCACTGGTACTACGCAAAGCCTAGTAACATCTTGAAACTCTTTTTGGTTGGTCGTTCC |
| | | ACTAGTTACCCCCTAGTAGACCTGGCAGATGAGGCAGGACGCTCCCCACTGGCGACA |
| | | GTGGTCCTGCCTGCGTGGCTGCCTGCACACCCTTCGGGGTGTGAAGCCAAAAGAAAG |
| | | ACAAGGTGTGAAGAGCCCCGTGTGCTACCAGTGAATCCTCCGGCCCCTGAATGCGGC |
| | | TAATCTTACCCCACAGCTATTGCACACAATCCAGTGTGTATGTAGTCGTAATGAGCA |
| | | ATTGTGGGACGGAACCGACTACTTTGGGTGTCCGTGTTTCCTTTTATTCCCATGTTTCT |
| | | GCTTATGGTGACAATACTGACGTATAGTGTTGTTACC |
| 1-4 | 4 | TTAAAACAGCGGATGGGTATCCCACCATCCGGCCCACTGGGTGTAGTACTCTGGTAC |
| | | ATTGTACCTTTGTACGCCTGTTTTCCCCCTCTTGTACCCGCCCTTCAAGCTCCTTGCCC |
| | | AAGTAACGTTAGAAGTTTGAACATTGGTACAATAGGAAGCATCACATCCAGTGGTGT |
| | | ACTGTACAAACACTTCTGTTGCCCCGGAGCGAGGTATAGATGGTCCCCACCGTCAAA |
| | | AGCCTTTAACCGTTATCCGCCAATCAACTACGTAATGGCTAGTAGCACCTTGGATTTA |
| | | AGTTGGCGTTCGATCAGGTGGTAACCCCCACTAGTTTGGTCGATGAGGCTAGGAATT |
| | | CCCCACGGGTGACCGTGTCCTAGCCTGCGTGGCGGCCAACCCAGCATCCGCTGGGAC |
| | | GCCAATTTAATGACATGGTGTGAAGACCTGCATGTGCTTGATTGTGAGTCCTCCGGC |
| | | CCCTGAATGCGGCTAACCCTAACCCCGGAGCCTTGCAGCACAATCCAGTGTTGTTAA |
| | | GGTCGTAATGAGCAATTCTGGGATGGGACCGACTACTTTGGGTGTCCGTGTTTCTTAT |
| | | TTTTCTTGAATTTTTCTTATGGTCACAGCATATATACATTATATACTGTGATC |
| 1-5 | 5 | TTAAAATAGCCTCAGGGTTGTTCCCACCCTGAGGGCCCACGTGGTGTAGTACTCTGG |
| | | TATTACGGTACCTTTGTACGCCTATTTTATACCCCCTTCCCCAAGTAATTTAGAAGCA |
| | | AGCACAAACCAGTTCAGTAGTAAGCAGTACAATCCAGTACTGTAATGAACAAGTACT |
| | | TCTGTTACCCCGGAAGGGTCTATCGGTAAGCTGTACCCACGGCTGAAGAATGACCTA |
| | | CCGTTAACCGGCTACCTACTTCGAGAAGCCTAGTAATGCCGTTGAAGTTTTATTGACG |
| | | TTACGCTCAGCACACTACCCCGTGTGTAGTTTTGGCTGATGAGTCACGGCACTCCCCA |
| | | CGGGCGACCGTGGCCGTGGCTGCGTTGGCGGCCAACCAAGGAGTGCAAGCTCCTTGG |
| | | ACGTCATATTACAGACATGGTGTGAAGAGCCTATTGAGCTAGGTGGTAGTCCTCCGG |
| | | CCCCTGAATGCGGCTAATCCTAACTCCGGAGCATATCGGTGCGAACCAGCACTTGGT |
| | | GTGTTGTAATACGTAAGTCTGGAGCGGAACCGACTACTTTGGGTGTCCGTGTTTCCTG |
| | | TTTTAACTTTTATGGCTGCTTATGGTGACAATTTAACATTGTTACCATATAGCTGTTG |
| | | GGTTGGCCATCCGGATTTTGTTATAAAACCATTTCCTCGTGCCTTGACCTTTAACACA |
| | | TTTGTGAACTTCTTTAAATCCCTTTTATTAGTCCTTAAATACTAAGA |
| 1-6 | 6 | TTCAAACAGCCTGGGGGTTGTACCCACCCCTGGGGCCCACGTGGCGCTAGTACTCTG |
| | | GTACGTTAGTACCTTTGTACGCCTGTTTTCCCCTCCCTTAAACAAATTAAGATTACCA |
| | | CTACTGAGGGGAGTAGTCCGACTCCGCTCCGGTACTGCCGCACCAGTACTCCGGTAC |
| | | ACTTAGTACCCTAGTACGGAGTAGATGGTATCCCCACCCCGCAACTTAGAAGCATGC |
| | | AAACAAACCGACCAATAGGCGCACGATATCCAGTCGTGTTTCGGTCAAGCACTTCTG |
| | | TCTCCCCGGTCCGAAAGGATCGTTACCCGCCCGACCCACTACGAGAAGCCCAGTAAC |
| | | TGGCCAAGTGATTGCGAAGTTGCGCTCAGCCACAACCCCAGTGGTAGCTCTGGAAGA |
| | | TGGGGCTCGCGTCTCCCCCGTGGTGACACGGTCGCTTGCCCGCGTGTGCTTCCGGGTT |
| | | CGGCCTACGCCGTTCACTTCAATGTCACGTAACCAGCCAAGAGCCTATTGTGCTGGG |
| | | ACGGTTTTCCTCCGGGGCCGTGAATGCTGCTAATCCCAACCTCCGAGCGTGTGCGCA |
| | | CAACCCAGTGTTGCTACGTCGTAATGCGTAAGTTGGAGGCGGAACAGACTACTTTCG |
| | | GTACCCCGTGTTTCCTTTAAATTTTATTCATTATTTTATGGTGACAATTGCTGAGATCT |
| | | GCGAATTAGCGACTCTGCCGTTGAATATTGCTCTGTACTATTTGGTTGCATTCCACAA |
| | | AACCTCTGACATCCCCAGTACATACATTACTTTACTTGTTTACCTCAATCTAAAGCAC |
| | | AAGCTAGATAATACAAA |
| 1-7 | 7 | TTTAAACAGCCTGGGGGTTGTTCCCACCCCTGGGGCCCACGTGGCGCTAGTACTCTG |
| | | GTACGCTAGTACCTTTGTACGCCTGTTTTTCCCCTCCCTTAAATAAATCAAGGTTGCC |
| | | ACTACTGAGGGGAGTAGTCCGACTCCGCTCCAGCAATGCTGCACCAGTGCACTGGTA |
| | | CGCTAGTACCTTTTCACGGAGTAGATGGTATCCCTTACCCCGGAACCTAGAAGATTG |
| | | CACACAAACCGACCAATAGGCGCACCGCATCCAGCCGTGCAGCGGTCAAGCACTTCT |
| | | GTCTCCCCGGTCTGTAAAGATCGTTATCCGCCCGACCCACTACGAAAAGCCTAGTAA |
| | | CTGGCCAAGTGAACGCGAAGTTGCGCTCCGCCACAACCCCAGTGGTAGCTCTGGAAG |
| | | ATGGGGCTCGCACCACCCCCGTGGTAACACGGTTGCCTGCCCGCGTGTGCTTCCGGG |
| | | TTCGGTCTCGTGCCGTTCACTTCAACTTCACGCAACCAGCCAAGAGCCTATTGTGCTG |
| | | GGACGGTTTTCCTCCGGGGCCGTGAATGCTGCTAATCCCAACCTCCGAGCGTGTGCG |
| | | CACAATCCAGTGTTGCTACGTCGTAACGCGTAAGTTGGAGGCGGAACAGACTACTTT |
| | | CGGTACCCCGTGTTTCCTCTCATTTTATTTAATATTTTATGGTGACAATTGTTGAGATT |
| | | TGCGCTCTTGCAACGTTGCCATTGAATATTGGCTTATACTATTTGGTTGCCTTTTACA |
| | | AAACCTCTGATATACCCAGTTCTTACATTGATCTGCTTGTTTTTCTCAATTTGAAGTAT |
| | | AGACTACAAATAGCAAA |
| 1-8 | 8 | CCCCCCTCCCCCCCTTCCCTTCCCTTTGCAACGCAACAATTGTAAGTGCCCTCACCTG |
| | | TCAATTGGGACCACCACTTTCAGTGACCCCATGCGAAGTGCTGAGAGAAAGGAAGCT |
| | | TTCTTACCCTTCATTTGTGAACCCACTGGTCTAAGCCGCTTGGAATACGATGAGTGGA |
| | | AAAGTTCATTCTTAATGGAGTGAAACATGCTTAAATTTCCAGCTCGTGCTGGTCTTTC |
| | | CAGTACGGGGCGGCCCTGTCTGGCCGTAATTCTTCAGAGTGTCACGCCACACTTGTG |
| | | GATCTCACGTGCCACATGACAGCGCTACAGCTGGAACTGGGTGCTTGGTGCCCATGG |
| | | AGTAACAGCGAAAAGTGTTAGATCAAGCCTTGCTTGGGCTATGAGCCTGCGGAACAA |
| | | CAACTGGTAACAGTTGCCTCAGGGGCCGAAAGCCACGGTGTTAACAGCACCCTCATA |
| | | GTTTGATCCACCTCAGGGTGGTGATGTTTAGCAGTTAGTAGTTGCCAATCTGTGTTCA |
| | | CTGAAATCTCGGCATACCGTGTAGTGTACAGGGGTGAAGGATGCCCAGAAGGTACCC |
| | | GTAGGTAACCTTAAGAGACTATGGATCTGATCTGGGGCCTTGTCCGGAGTGCTTTAC |
| | | ACACGGCTCAAGGTTAAAAAACGTCTAGCCCCACAGAGCCCGAGGGATTCGGGTTTT |
| | | CCCTTTAAAAACCCGACTAGAGCTTATGGTGACAATTATTGCTGTTCAGACGAACAG |
| | | TGTAATTGTTGTCTATTCACAGCAGTTCTATCAGAGCTTTTCCCACAACGGATCTTCT |
| | | TGGCAAGCAAATACAGCAGGAGTCAAT |
| 1-9 | 9 | CACTACGTTACGGTTCCCGCCCGGGACAACTGGTACCCCATTAGGCTACAACATGGC |
| | | TGAAAAGGGTATTGGGTCCCCCCGGATTGTGTCCGTTCGTAGTGTGTGTAACGTGGTT |
| | | TACCATCTCCACTAACATTGGACTAAGCATTTCATCTTTCCTCCCCGATTGTGTACTC |
| | | ACTTGGCTAACGCTGGGTGGTCGCGGTTGGGTCCTTGATTTACTTTTTCTCGTCTAAG |
| | | CATTCCGACTGTCCTCCCCGATTATGTGCTCATTCAGTTAACTGCTGGGTGGTCATGA |
| | | CTAACATCGAGGAACCTTCTGTCCACGCTTACTTTGAGCTCCGGTCGCTTGACGCTTG |
| | | TAGGGCGATAGGGTTATCTTCCTGACAACATCTTTATTCTACCTCCATAGGCTCTATC |
| | | TATGGAGACGGAGTGTGGCACCCGTCCCTTCTTTGGGAGCTTCGGTAGTGACGCCCT |
| | | TTGTCACTCTCGCCAGCCGAGGCATGCCTGGTGCCAGGTAGCAAAGAAAGCATATGT |
| | | TTAAGGACTTGACTGATTTAGCGCAAGAGTTTGTAGCGATGTCCATAGTGTCTGCGG |
| | | ATTCCCCACACGGCGACGTGTGCCGCGGAGGCCAAAAGCCACGGTGTTCACAGCACC |
| | | CCTATGGATGCCCACAGACCCCAGTGGGCACTCTTGTTGCCGGACTTTCAGGAAATT |
| | | AGGCATAGGCTCTTCTCAAACTCCTGGCATTGGACTAGGTAAGAATGCCCCGGAGGT |
| | | ACCCCAGTACTCCTTCGGGAGTCTGGGATCTGACCGGGGGCCCCACAAACATGCTTT |
| | | ACGTGTTTCGTGCGGTCAAAAATTGTCTAACTAGTCCCAACCTTGAACAAGGGATTG |
| | | TTCTTTCCTTTTTATTACTGAGACTGGCCTATGGTGACAACAGAGATTGACTGTGAAT |
| | | ACAGTTATTTTCTGGTGTTTATCATTTGGTTTTTCTCCGTGCTCTTTTACCTTTGTGGTA |
| | | TTTGTTCTTTAGATAGGCAAA |
| 1-10 | 10 | CCCGGCCACCCCCTTTCGACGCGGGTACTGCGATAGTGCCACCCCAGTCTTTCCTACT |
| | | CCCGACTCCCGACTCTAACCCAGGTTCCTTGGAACAGGAACACCAATATACTCATCC |
| | | CCTGGATGCTGACTAATCAGAGGAACGTCAGCATTTTCCGGCCCAGGCTAAGAGAAG |
| | | TAGATAAGTTAGATTCCAAATTGATTTATCATCCCCTTGACGAATTCGCGTTGGAAAT |
| | | GCACCTCTCACTTGCCGCTCTTCACACCCATTAACTTGATTCGGCCTCTGTGTTGAGC |
| | | CCCTTGTTGAAGTGCTTCCCTCCATCGTGACGTGGTTGGAGATCTAAGTCAACCGACT |
| | | CCGACGAAACTACCATCATGCCTCCCCGATTATGTGATGCTTTCTGCCCTGCTGGGTG |
| | | GAGCATCCTCGGGTTGAGAAAACCTTCTTCCTTTTTCCTTGGACCCCGGTCCCCCGGT |
| | | CTAAGCCGCTTGGAATAAGACAGGGTTATCTTCACCTCTTCCTTCTTCTACTTCATAG |
| | | TGTTCTATACTATGAAAGGGTATGTGTCGCCCCTTCCTTCTTTGGAGAACACGCGCGG |
| | | CGGTCTTTCCGTCTCTCGAAAAGCGCGTGTGCGACATGCAGAGAACCGTGAAGAAAG |
| | | CAGTTTGCGGACTAGCTTTAGTGCCCACAAGAAAACAGCTGTAGCGACCACACAAAG |
| | | GCAGCGGACCCCCCCTCCTGGCAACAGGAGCCTCTGCGGCCAAAAGCCACGTGGAT |
| | | AAGATCCACCTTTGTGTGCGGCACAACCCCAGTGCCCTGGTTTCTTGGTGACACTTCA |
| | | GTGAAAACGCAAATGGCGATCTGAAGCGCCTCTGTAGGAAAGCCAAGAATGTCCAG |
| | | GAGGTACCCCTTCCCTCGGGAAGGGATCTGACCTGGAGACACATCACATGTGCTTTA |
| | | CACCTGTGCTTGTGTTTAAAAATTGTCACAGCTTTCCCAAACCAAGTGGTCTTGGTTT |
| | | TCACTCTTTAAACTGATTTCACT |
| 1-11 | 11 | CCCCCGGTTACCCCCTTTCGACGCGGGTACTGCGATAGTGCCACCCCAGTCTTTCCTA |
| | | CTCCCGACTCCCGACCCTAACCCAGGTTCCTCGGAACAGGAACACAAATTTACTCAT |
| | | CCCCTGGATGCTGACTAATCAGAGGAACGTCAGCATTTTCCGGCCCAGGCTTAGAGA |
| | | AGTAGATAAGTTAGAATCTAAATTGATATGACTTCCCCTTGACGAATTCACGTTGGA |
| | | AATGCACCCCTCACTTGCCGCTCTTCACACCCACTAATTGATTCGGCCTACTGTGTTG |
| | | AGCCCCTTGTTGAAGTGCTTCCCTCCCTCGTGACGTGGTTGGAGAAATCTTGTCACCC |
| | | GACTCCGACGAAACTACCATCATGCCTCCCCGATTATGTGATGCTTTCTGCCCTGCTG |
| | | GGTGGAGTATCCTCGGGTTGAGAAATCCTTCTTCCTTTTACCTTGGACCTTGGTCCCC |
| | | CGGTCTAAGCCGCTTGGAATAAGACAGGGTTATTTTCACCTCTTCTTCTTCTACTTCA |
| | | TGGTGCTCTATACCATGAAAGGGTATGTGTCGCCCCTTCCTTCTTGGAGAACTCACGC |
| | | GGCGGTCTTCCGTCTCTCAAAAAGCGCGAGTGCGACATGCAGAGTAACGCGAAGAA |
| | | AGCAGTTCCTGGCCTAGCTCTAGTGCCCACAAGAAAACGGCTGTAGCGACCACACAA |
| | | AGGCAGCGGAACTCCCCTCCTGGTAACAGGAGCCTCTGCGGCCAAAAGCCACGTGG |
| | | ATTAGATCCACCTTTGTGTGCGGTGCAACCCCAGCACCCCGGTTTCTTGTTGACACTC |
| | | TAGTGAATCCTTGAATGGCAATCTCAAGCGCCTCTGTAGGAAAGCCAAGAATGTCCA |
| | | GGAGGTACCCCTTCCTCGCGGAAGGGATCTGACCTGGAGACACATCACACGTGCTTT |
| | | ACACTTGTGCTTGTGTTTAAAAATTGTCACAGCTTTCCCAAACCAAGTGGTCTTGGTT |
| | | TTCCTTTTTTATCCTACTGTCAAT |
| 1-12 | 12 | TCCTCACCCATGCTTTTCCTACCCCCACCACGCCCGCATGTTTACTGCTTTCCTTGATG |
| | | CTGCCCGTGACTACTTCCATGACCTCCCCAACCCAAACCTCAAACGCCTTAAGCTATC |
| | | ATGGGCTCTCTATCACCAATCCCCTTCCTTCCCACCCAGAACCCCCCCCTCCCTCCCC |
| | | TACAACGTGTATGAGCAAGACGAGTTTGACAAGCTCGCCGAAGCCATGCTCACCCAC |
| | | TCTCCCTTCCCCACATCCCATTACCTCTTCCACCCCCTCCCGTCCAACGCCCGCACCA |
| | | TCGTCGAGCGTGAGGCTGACTGGGATGGCTGTGATCTAGAAAAGAAATGGCTCGACC |
| | | TCGTCATAGAGGACGATGCCAAGTTCCTTCTGGAGAACGGCTCTCTCCCGTTTGGCTC |
| | | CACCCTTGCC |
| 1-13 | 13 | TTCTCGCCTGAGTCAACAAAGCGAGAAACCTGCCCCTCCAGCGCCAGACGAGCGGCA |
| | | TAAAACTTGAACTTCTGGCATGCTCCACCACCCTTTTCCCCATTCCAACCCCCATTGC |
| | | GCTCTCAAGGTCGCGCTTTTTCGAGACTAGCTCGGATTCAAAAGTTCCTGGCACCCTT |
| | | TGCCCCTTCAGGCCCTTAAGGTAGGAACTGACCTTGTGCTGTGCCCTCGGTGCGGAA |
| | | GTGCTACTGCGTAGCGATTGTAAGATCCCTTTGTGGTTCTGCCCTGGCAAGGTTATAG |
| | | AGTACTGTGATCCGCTGCGGATGCCATCCTGGTAACAGGACCCCCAGTGTGCGCAAC |
| | | AGTATGTTCACGGTCTTCCGTGTCCACCACATTCGGAACACTGCTCTCGTGAAACAGT |
| | | GTGTGTCCAATCCCTGCAATCAGTATCAACTACACCACCTAGGAATGCTAGGAAGGT |
| | | ACCCCGGTCCGCCGGGATCTGATCCTAGGCTAATTGTCTACGGTGGTGCTCCTTTTTA |
| | | TTTTCCACTTCAATTCATTGGTTACAACTGCTCGATCCCTGTGTTTGCTGCCCTTCTCT |
| | | GCTCTCATCGCCATTCTCAAGTGTTCACACTGTCCAAGTTCCTTTGGTTGTTCGCTTCC |
| | | ACTTGCCACTGTCAACTCTTGTC |
| 1-14 | 14 | TCACCCTCTTTTCCGGTGGTCCGGACCCAGACCACCGTTACTCCATTCAGCTTCTTCG |
| | | GAACCTGTTTGGAGGAATTAAACGGGCACCCACCCACCTTCACCCCCTTTTCGTAACT |
| | | AAGTGTGTGCCCAATCTCATGACTCCTGCTGACTTCACCGACCAGCAGTGTCCAAAA |
| | | CGCTAGGTGAATTTCCTTCCTCCCCCTCTGGGCTTCTGCCCAGCTCCCTCCCTCCAGC |
| | | CTGACGTGCCACAGGCTGTGCAAAGACCCCGCGAAAGCTGCCAAAAGTGGCAATTG |
| | | TGGGTCCCCCCTTTGTAAAGGCGTCGAGTCTTTCTCCCTTAAGGCTAGACCCGTCAGT |
| | | GAATTCTGTCGGGCAACTAGTGACGCCACTGCATGCCTCCGACCTCGGCCGCGGAGT |
| | | GCTGCCCCCCAAGTCGTGCCCCTGACTACAAGTTGTGCTGTCTGGCAAACATTGTCTG |
| | | TGAGAATGTTCCGCTGTGGCTGCCAAGCCTGGTAACAGGCTGCCCCAGTGTGCGTAG |
| | | TTCTCATCCAGACTTCGGTCTGGCAACTTGCTGTTAAGACACGGCGTAAGGGGCGTG |
| | | TGCCAACGCCCTGGAACGAGTGTCCACTCTAATACCCCGAGGAATGCTACGCAGGTA |
| | | CCCCTGGCTCCCCAGGGATCTGAGCGTAGGCTAATTGTCTAAGGGTATTTTCATTTCC |
| | | CACTCTTTCTTTCTTGTTCATA |
| 1-15 | 15 | TCTGTCCTCACCCCATCTTCCCTTCTTTCCTGCACCGTTACGCTTACTCGCATGTGCAT |
| | | TGAGTGGTGCACGTGCTTGAACAAACAGCTACACTCACATGGGGGCGGGTTTTCCCG |
| | | CCCTGCGGCCTCTCGCGAGGCCCACCCCTCCCCTTCCTCCCATAACTACAGTGCTTTG |
| | | GTAGGTAAGCATCCTGATCCCCCGCGGAAGCTGCTCACGTGGCAACTGTGGGGACCC |
| | | AGACAGGTTATCAAAGGCACCCGGTCTTTCCGCCTTCAGGAGTATCCCTGCTAGTGA |
| | | ATTCTAGTAGGGCTCTGCTTGGTGCCAACCTCCCCCAAATGCGCGCTGCGGGAGTGC |
| | | TCTTCCCCAACTCACCCTAGTATCCTCTCATGTGTGTGCTTGGTCAGCATATCTGAGA |
| | | CGATGTTCCGCTGTCCCAGACCAGTCCAGTAATGGACGGGCCAGTGTGCGTAGTCGT |
| | | CTTCCGGCTTGTCCGGCGCATGTTTGGTGAACCGGTGGGGTAAGGTTGGTGTGCCCA |
| | | ACGCCCGTACTTTGGTGATACCTCAAGACCACCCAGGAATGCCAGGGAGGTACCCCG |
| | | CTTCACAGCGGGATCTGACCCTGGGCTAATTGTCTACGGTGGTTCTTCTTGCTTCCAC |
| | | TTCTTTCTACTGTTCATG |
| 1-16 | 16 | ATTCTCGGGCTACGGCCCTGGAGCCACTCCGGCTCCTAAAGATTTAGAAGTTTGAGC |
| | | ACACCCGCCCACTAGGGCCCCCCATCCAGGGGGGCAACGGGCAAGCACTTCTGTTTC |
| | | CCCGGTATGATCTGATAGGCTGTAACCACGGCTGAAACAGAGATTATCGTTATCCGC |
| | | TTCACTACTTCGAGAAGCCTAGTAATGATGGGTGAAATTGAATCCGTTGATCCGGTG |
| | | TCTCCCCCACACCAGAAACTCATGATGAGGGTTGCCATCCCGGCTACGGCGACGTAG |
| | | CGGGCATCCCTGCGCTGGCATGAGGCCTCTTAGGAGGACGGATGATATGGATCTTGT |
| | | CGTGAAGAGCCTATTGAGCTAGTGTCGACTCCTCCGCCCCCGTGAATGCGGCTAATC |
| | | CTAACCCCGGAGCAGGTGGGTCCAATCCAGGGCCTGGCCTGTCGTAATGCGTAAGTC |
| | | TGGGACGGAACCGACTACTTTCGGGAAGGCGTGTTTCCATTTGTTCATTATTTGTGTG |
| | | TTTATGGTGACAACTCTGGGTAAACGTTCTATTGCGTTTATTGAGAGATTCCCAACAA |
| | | TTGAACAAACGAGAACTACCTGTTTTATTAAATTTACACAGAGAAGAATTACA |
| 1-17 | 17 | IGTGGCCACGCCCGGGCCACCGATACTTCCCTTCACTCCTTCGGGACTGTTGGGGAGG |
| | | AACACAACAGGGCTCCCCTGTTTTCCCATTCCTTCCCCCTTTTCCCAACCCCAACCGC |
| | | CGTATCTGGTGGCGGCAAGACACACGGGTCTTTCCCTCTAAAGCACAATTGTGTGTG |
| | | TGTCCCAGGTCCTCCTGCGTACGGTGCGGGAGTGCTCCCACCCAACTGTTGTAAGCCT |
| | | GTCCAACGCGTCGTCCTGGCAAGACTATGACGTCGCATGTTCCGCTGCGGATGCCGA |
| | | CCGGGTAACCGGTTCCCCAGTGTGTGTAGTGCGATCTTCCAGGTCCTCCTGGTTGGCG |
| | | TTGTCCAGAAACTGCTTCAGGTAAGTGGGGTGTGCCCAATCCCTACAAAGGTTGATT |
| | | CTTTCACCACCTTAGGAATGCTCCGGAGGTACCCCAGCAACAGCTGGGATCTGACCG |
| | | GAGGCTAATTGTCTACGGGTGGTGTTTCCTTTTTCTTTTCACACAACTCTACTGCTGA |
| | | CAACTCACTGACTATCCACTTGCTCTGTCACG |
| 1-18 | 18 | TTTGCTCAGCGTAACTTCTCCGGGTTACGTGGAGACCAAAAGGCTACGGAGACTCGG |
| | | GCTACGGCCCTGGAGCACCTAGGTGCTCCTAAAGACGTTAGAAGTTGTACAAACTCG |
| | | CCCAATAGGGCCCCCCAACCAGGGGGGTAGCGGGCAAGCACTTCTGTTTCCCCGGTA |
| | | TGATCTCATAGGCTGTACCCACGGCTGAAAGAGAGATTATCGTTACCCGCCTCACTA |
| | | CTTCGAGAAGCCCAGTAATGGTTCATGAAGTTGATCTCGTTGACCCGGTGTTTCCCCC |
| | | ACACCAGAAACCTGTGATGGGGGTGGTCATCCCGGTCATGGCGACATGACGGACCTC |
| | | CCCGCGCCGGCACAGGGCCTCTTCGGAGGACGAGTGACATGGATTCAACCGTGAAG |
| | | AGCCTATTGAGCTAGTGTTGATTCCTCCGCCCCCGTGAATGCGGCTAATCCCAACTCC |
| | | GGAGCAGGCGGGCCCAAACCAGGGTCTGGCCTGTCGTAACGCGAAAGTCTGGAGCG |
| | | GAACCGACTACTTTCGGGAAGGCGTGTTTCCTTTTGTTCCTTTTATCAAGTTTTATGGT |
| | | GACAACTCCTGGTAGACGTTTTATTGCGTTTATTGAGAGATTTCCAACAATTGAACAG |
| | | ACTAGAACCACTTGTTTTATCAAACCCTCACAGAATAAGATAACA |

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to an IRES sequence in Table IA, an IRES sequence from a construct of SEQ ID NOs: 50-61, or shown below for any of Constructs A-P of Table IB, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR shown below for any of Constructs A-P of Table 1B. In some embodiments, said circular RNA further comprises a CD28z or 4-1BB costimulatory domain as described herein.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct A, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct A. In some embodiments, the circular RNA comprises an IRES sequence having at least 95%, 96%, 97%, 98%, 99%, or 100% sequence identity to the IRES sequence of Construct A, and a CAR sequence encoding a polypeptide having at least 95%, 96%, 97%, 98%, 99%, or 100% sequence identity to a CAR of Construct A. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct B, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct B. In some embodiments, the circular RNA comprises an IRES sequence having at least 95%, 96%, 97%, 98%, 99%, or 100% sequence identity to the IRES sequence of Construct B, and a CAR sequence encoding a polypeptide having at least 95%, 96%, 97%, 98%, 99%, or 100% sequence identity to a CAR of Construct B. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct C, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct C. In some embodiments, the circular RNA comprises an IRES sequence having at least 95%, 96%, 97%, 98%, 99%, or 100% sequence identity to the IRES sequence of Construct C, and a CAR sequence encoding a polypeptide having at least 95%, 96%, 97%, 98%, 99%, or 100% sequence identity to a CAR of Construct C. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct D, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct D. In some embodiments, the circular RNA comprises an IRES sequence having at least 95%, 96%, 97%, 98%, 99%, or 100% sequence identity to the IRES sequence of Construct D, and a CAR sequence encoding a polypeptide having at least 95%, 96%, 97%, 98%, 99%, or 100% sequence identity to a CAR of Construct D. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct E, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct E. In some embodiments, the circular RNA comprises an IRES sequence having at least 95%, 96%, 97%, 98%, 99%, or 100% sequence identity to the IRES sequence of Construct E, and a CAR sequence encoding a polypeptide having at least 95%, 96%, 97%, 98%, 99%, or 100% sequence identity to a CAR of Construct E. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct F, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct F. In some embodiments, the circular RNA comprises an IRES sequence having at least 95%, 96%, 97%, 98%, 99%, or 100% sequence identity to the IRES sequence of Construct F, and a CAR sequence encoding a polypeptide having at least 95%, 96%, 97%, 98%, 99%, or 100% sequence identity to a CAR of Construct F. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct G, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct G. In some embodiments, the circular RNA comprises an IRES sequence having at least 95%, 96%, 97%, 98%, 99%, or 100% sequence identity to the IRES sequence of Construct G, and a CAR sequence encoding a polypeptide having at least 95%, 96%, 97%, 98%, 99%, or 100% sequence identity to a CAR of Construct G. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct H, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct H. In some embodiments, the circular RNA comprises an IRES sequence having at least 95%, 96%, 97%, 98%, 99%, or 100% sequence identity to the IRES sequence of Construct H, and a CAR sequence encoding a polypeptide having at least 95%, 96%, 97%, 98%, 99%, or 100% sequence identity to a CAR of Construct H. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct I, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct I. In some embodiments, the circular RNA comprises an IRES sequence having at least 95%, 96%, 97%, 98%, 99%, or 100% sequence identity to the IRES sequence of Construct I, and a CAR sequence encoding a polypeptide having at least 95%, 96%, 97%, 98%, 99%, or 100% sequence identity to a CAR of Construct I. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct J, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct J. In some embodiments, the circular RNA comprises an IRES sequence having at least 95%, 96%, 97%, 98%, 99%, or 100% sequence identity to the IRES sequence of Construct J, and a CAR sequence encoding a polypeptide having at least 95%, 96%, 97%, 98%, 99%, or 100% sequence identity to a CAR of Construct J. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct K, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct K. In some embodiments, the circular RNA comprises an IRES sequence having at least 95%, 96%, 97%, 98%, 99%, or 100% sequence identity to the IRES sequence of Construct K, and a CAR sequence encoding a polypeptide having at least 95%, 96%, 97%, 98%, 99%, or 100% sequence identity to a CAR of Construct K. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct L, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct L. In some embodiments, the circular RNA comprises an IRES sequence having at least 95%, 96%, 97%, 98%, 99%, or 100% sequence identity to the IRES sequence of Construct L, and a CAR sequence encoding a polypeptide having at least 95%, 96%, 97%, 98%, 99%, or 100% sequence identity to a CAR of Construct L. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct M, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct M. In some embodiments, the circular RNA comprises an IRES sequence having at least 95%, 96%, 97%, 98%, 99%, or 100% sequence identity to the IRES sequence of Construct M, and a CAR sequence encoding a polypeptide having at least 95%, 96%, 97%, 98%, 99%, or 100% sequence identity to a CAR of Construct M. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct N, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct N. In some embodiments, the circular RNA comprises an IRES sequence having at least 95%, 96%, 97%, 98%, 99%, or 100% sequence identity to the IRES sequence of Construct N, and a CAR sequence encoding a polypeptide having at least 95%, 96%, 97%, 98%, 99%, or 100% sequence identity to a CAR of Construct N. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct O, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct O. In some embodiments, the circular RNA comprises an IRES sequence having at least 95%, 96%, 97%, 98%, 99%, or 100% sequence identity to the IRES sequence of Construct O, and a CAR sequence encoding a polypeptide having at least 95%, 96%, 97%, 98%, 99%, or 100% sequence identity to a CAR of Construct O. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of Construct P, and a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of Construct P. In some embodiments, the circular RNA comprises an IRES sequence having at least 95%, 96%, 97%, 98%, 99%, or 100% sequence identity to the IRES sequence of Construct P, and a CAR sequence encoding a polypeptide having at least 95%, 96%, 97%, 98%, 99%, or 100% sequence identity to a CAR of Construct P. In some embodiments, said circular RNA exhibits increased expression and/or activity compared to a suitable control having an alternate IRES. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of SEQ ID NO: 8 and a sequence encoding a CAR polypeptide. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of SEQ ID NO: 8 and a sequence encoding a HER2 CAR polypeptide. In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of SEQ ID NO: 8 and a sequence encoding a CD19 CAR polypeptide. In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of SEQ ID NO: 8 and a sequence encoding a BCMA polypeptide. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of SEQ ID NO: 18 and a sequence encoding a CAR polypeptide. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of SEQ ID NO: 18 and a sequence encoding a HER2 CAR polypeptide. In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of SEQ ID NO: 18 and a sequence encoding a CD19 CAR polypeptide. In some embodiments, the circular RNA comprises an IRES sequence having at least 85%, 86%, 87%, 88%, 89%, 90%, 91%, 92%, 93%, 94%, 95%, 96%, 97%, 98%, 99%, or 100% sequence identity to the IRES sequence of SEQ ID NO: 18 and a sequence encoding a BCMA polypeptide. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased.

In some embodiments, the circular RNA comprises a CAR sequence encoding a polypeptide having at least 85%, 86%, 87%, 88%, 89%, 90%, 91%, 92%, 93%, 94%, 95%, 96%, 97%, 98%, 99%, or 100% sequence identity to a CAR of any one of Constructs A-P of Table IB or binding fragments thereof. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

TABLE 1B
Exemplary Constructs (DNA Templates)

| | | | CAR |
|---|---|---|---|
| ID | IRES Sequence | CAR Sequence | Sequence |
| Construct | CCCCCCTCCCCCCCTTCC | ATGGCTCTCCCCGTGACCGCTCTGCTGC | MALPVTALLL |
| A | CTTCCCTTTGCAACGCAA | TCCCTCTGGCCCTCCTTCTGCACGCAGC | PLALLLHAAR |
| | CAATTGTAAGTGCCCTCA | CAGACCACAGGTCAAGCTGGAGGAGTC | PQVKLEESGG |
| | CCTGTCAATTGGGACCAC | TGGTGGCGGTCTGGTGCAGGCAGGGAG | GLVQAGRSLR |
| | CACTTTCAGTGACCCCAT | GAGCCTGAGGCTGAGCTGTGCAGCTTCC | LSCAASEHTFS |
| | GCGAAGTGCTGAGAGAA | GAGCACACATTCTCAAGCCACGTCATGG | SHVMGWFRQ |
| | AGGAAGCTTTCTTACCCT | GGTGGTTCAGACAGGCTCCCGGTAAAG | APGKERESVA |
| | TCATTTGTGAACCCACTG | AGAGGGAGTCCGTCGCCGTGATCGGATG | VIGWRDISTSY |
| | GTCTAAGCCGCTTGGAAT | GCGGGACATCTCCACCTCCTACGCCGAC | ADSVKGRFTIS |
| | ACGATGAGTGGAAAAGTT | TCTGTGAAGGGCCGGTTCACAATCTCAC | RDNAKKTLYL |
| | CATTCTTAATGGAGTGAA | GCGATAATGCCAAGAAGACACTGTATCT | QMNSLKPEDT |
| | ACATGCTTAAATTTCCAG | GCAGATGAATTCCTTGAAGCCCGAAGAC | AVYYCAARRI |
| | CTCGTGCTGGTCTTTCCA | ACCGCCGTCTATTACTGTGCTGCTAGAC | DAADFDSWG |
| | GTACGGGGCGGCCCTGTC | GGATCGACGCTGCCGACTTCGACAGCTG | QGTQVTVSSG |
| | TGGCCGTAATTCTTCAGA | GGGACAGGGTACCCAAGTGACCGTTTCC | GGGSGGGGSG |
| | GTGTCACGCCACACTTGT | TCCGGAGGCGGAGGTTCTGGAGGAGGT | GGGSEVQLVE |
| | GGATCTCACGTGCCACAT | GGGTCAGGTGGAGGTGGCTCCGAGGTG | SGGGLVQAGG |
| | GACAGCGCTACAGCTGG | CAGCTGGTCGAGTCTGGCGGTGGCTTGG | SLRLSCAASG |
| | AACTGGGTGCTTGGTGCC | TCCAGGCTGGAGGCAGTCTCAGACTCTC | RTFTMGWFRQ |
| | CATGGAGTAACAGCGAAA | CTGCGCTGCTTCAGGGCGGACCTTCACC | APGKEREFVA |
| | AGTGTTAGATCAAGCCTT | ATGGGCTGGTTCAGGCAGGCCCCAGGTA | AISLSPTLAYY |
| | GCTTGGGCTATGAGCCTG | AGGAGAGGGAGTTCGTGGCCGCCATCTC | AESVKGRFTIS |
| | CGGAACAACAACTGGTA | CCTCTCCCCTACCCTGGCATACTACGCTG | RDNAKNTVVL |
| | ACAGTTGCCTCAGGGGCC | AGTCCGTGAAGGGACGGTTTACCATCTC | QMNSLKPEDT |
| | GAAAGCCACGGTGTTAAC | CCGGGATAACGCAAAGAACACTGTGGT | ALYYCAADRK |
| | AGCACCCTCATAGTTTGA | CCTCCAAATGAACTCCCTCAAACCCGAG | SVMSIRPDYW |
| | TCCACCTCAGGGTGGTGA | GACACCGCTCTCTACTATTGTGCCGCAG | GQGTQVTVSS |
| | TGTTTAGCAGTTAGTAGT | ATCGGAAGAGCGTCATGTCCATCCGGCC | TSTTTPAPRPP |
| | TGCCAATCTGTGTTCACT | CGATTACTGGGGCCAAGGCACACAGGT | TPAPTIASQPL |
| | GAAATCTCGGCATACCGT | GACTGTGTCCAGCACCTCCACCACCACC | SLRPEACRPAA |
| | GTAGTGTACAGGGGTGAA | CCAGCACCAAGGCCTCCAACCCCTGCA | GGAVHTRGLD |
| | GGATGCCCAGAAGGTACC | CCAACCATCGCCTCCCAGCCACTGTCTT | FACDIYIWAPL |
| | CGTAGGTAACCTTAAGAG | TGCGGCCAGAAGCATGCCGCCCAGCAG | AGTCGVLLLS |
| | ACTATGGATCTGATCTGG | CAGGTGGAGCCGTGCATACAAGAGGCC | LVITLYCKRGR |
| | GGCCTTGTCCGGAGTGCT | TGGACTTCGCCTGCGATATCTACATCTGG | KKLLYIFKQPF |
| | TTACACACGGCTCAAGGT | GCTCCTCTGGCCGGAACATGCGGAGTCC | MRPVQTTQEE |
| | TAAAAAACGTCTAGCCCC | TGCTCTTGTCCCTGGTGATCACCCTGTAC | DGCSCRFPEEE |
| | ACAGAGCCCGAGGGATTC | TGCAAGCGGGGTCGGAAGAAGCTCCTC | EGGCELRVKF |
| | GGGTTTTCCCTTTAAAAA | TACATCTTCAAGCAGCCCTTCATGAGAC | SRSADAPAYQ |
| | CCCGACTAGAGCTTATGG | CCGTCCAGACCACCCAGGAGGAGGACG | QGQNQLYNEL |
| | TGACAATTATTGCTGTTCA | GGTGCTCATGCAGGTTCCCCGAAGAGGA | NLGRREEYDV |
| | GACGAACAGTGTAATTGT | GGAGGGTGGCTGTGAGCTGCGGGTGAA | LDKRRGRDPE |
| | TGTCTATTCACAGCAGTT | GTTCAGCAGGTCAGCAGACGCCCCTGCC | MGGKPRRKNP |
| | CTATCAGAGCTTTTCCCA | TATCAGCAGGGCCAAAACCAGTTGTACA | QEGLYNELQK |
| | CAACGGATCTTCTTGGCA | ACGAGCTGAATCTGGGGAGACGGGAGG | DKMAEAYSEI |
| | AGCAAATACAGCAGGAGT | AGTACGATGTCCTTGACAAGAGAAGGG | GMKGERRRG |
| | CAAT (SEQ ID NO: 8) | GCCGGGATCCAGAGATGGGCGGGAAGC | KGHDGLYQGL |
| | | CAAGACGGAAGAATCCTCAGGAGGGTC | STATKDTYDA |
| | | TGTATAACGAGCTGCAGAAGGACAAGAT | LHMQALPPR |
| | | GGCCGAGGCCTACTCCGAGATCGGCATG | (SEQ ID NO: |
| | | AAAGGGGAGCGCCGCAGAGGAAAAGGT | 122) |
| | | CACGATGGTCTGTACCAGGGGTTGAGCA | |
| | | CCGCTACCAAGGATACTTACGACGCTCT | |
| | | GCACATGCAAGCTCTGCCACCCCGG | |
| | | (SEQ ID NO: 106) | |
| Construct | GTGGCCACGCCCGGGCC | ATGGCACTCCCGGTAACCGCCTTATTGCT | MALPVTALLL |
| B | ACCGATACTTCCCTTCAC | TCCCCTTGCCCTCTTGCTCCACGCAGCA | PLALLLHAAR |
| | TCCTTCGGGACTGTTGGG | CGCCCCGATATAGTCTTGACTCAATCCCC | PDIVLTQSPPS |
| | GAGGAACACAACAGGGC | ACCCAGTTTGGCAATGTCATTAGGCAAA | LAMSLGKRAT |
| | TCCCCTGTTTTCCCATTCC | CGAGCAACAATTTCATGTAGGGCATCCG | ISCRASESVTIL |
| | TTCCCCCTTTTCCCAACC | AAAGTGTAACGATTTTGGGGAGTCATTT | GSHLIHWYQQ |
| | CCAACCGCCGTATCTGGT | AATTCATTGGTACCAACAAAAGCCTGGA | KPGQPPTLLIQ |
| | GGCGGCAAGACACACGG | CAACCCCCGACGCTCTTGATCCAATTAG | LASNVQTGVP |
| | GTCTTTCCCTCTAAAGCA | CATCTAACGTCCAAACCGGAGTCCCCGC | ARFSGSGSRTD |
| | CAATTGTGTGTGTGTCCC | ACGATTCTCAGGATCCGGTTCCCGGACT | FTLTIDPVEED |
| | AGGTCCTCCTGCGTACGG | GATTTTACATTAACTATTGATCCGGTAGA | DVAVYYCLQS |
| | TGCGGGAGTGCTCCCACC | GGAAGATGACGTCGCTGTCTATTATTGTC | RTIPRTFGGGT |
| | CAACTGTTGTAAGCCTGT | TTCAAAGTAGGACGATTCCACGGACATT | KLEIKGSTSGS |
| | CCAACGCGTCGTCCTGGC | CGGTGGCGGAACTAAATTGGAGATTAAA | GKPGSGEGST |
| | AAGACTATGACGTCGCAT | GGTTCCACCTCTGGTAGTGGGAAACCCG | KGQIQLVQSGP |
| | GTTCCGCTGCGGATGCCG | GGTCCGGTGAAGGGTCCACTAAAGGCC | ELKKPGETVKI |
| | ACCGGGTAACCGGTTCCC | AAATTCAACTCGTTCAATCCGGACCAGA | SCKASGYTFT |
| | CAGTGTGTGTAGTGCGAT | ACTGAAGAAGCCAGGAGAAACTGTCAA | DYSINWVKRA |
| | CTTCCAGGTCCTCCTGGT | AATAAGCTGTAAAGCTTCCGGTTATACAT | PGKGLKWMG |
| | TGGCGTTGTCCAGAAACT | TTACAGATTATTCCATAAATTGGGTGAAA | WINTETREPAY |
| | GCTTCAGGTAAGTGGGGT | AGGGCGCCAGGAAAAGGGTTAAAGTGG | AYDFRGRFAFS |
| | GTGCCCAATCCCTACAAA | ATGGGTTGGATTAATACAGAGACTCGGG | LETSASTAYLQ |
| | GGTTGATTCTTTCACCAC | AACCTGCATATGCTTATGATTTTAGGGGA | INNLKYEDTAT |
| | CTTAGGAATGCTCCGGAG | AGGTTTGCCTTTTCTCTGGAGACTTCCG | YFCALDYSYA |
| | GTACCCCAGCAACAGCTG | CTTCAACTGCTTATCTCCAAATTAATAAT | MDYWGQGTS |
| | GGATCTGACCGGAGGCTA | CTTAAATATGAGGACACAGCAACATACT | VTVSSAAATT |
| | ATTGTCTACGGGTGGTGT | TCTGTGCTTTGGACTATAGTTATGCTATG | TPAPRPPTPAP |
| | TTCCTTTTTCTTTTCACAC | GATTACTGGGGACAAGGAACCAGTGTC | TIASQPLSLRP |
| | AACTCTACTGCTGACAAC | ACTGTAAGTTCCGCTGCTGCGACGACCA | EACRPAAGGA |
| | TCACTGACTATCCACTTG | CTCCTGCACCGCGACCACCCACTCCTGC | VHTRGLDFAC |
| | CTCTGTCACG (SEQ ID NO: | CCCTACTATTGCTAGTCAACCACTTAGCT | DIYIWAPLAGT |
| | 17) | TGCGACCTGAGGCATGTCGGCCCGCGGC | CGVLLLSLVIT |
| | | AGGTGGCGCAGTCCACACCAGGGGTTT | LYCKRGRKKL |
| | | AGACTTTGCTTGTGATATTTATATTTGGG | LYIFKQPFMRP |
| | | CACCACTCGCCGGGACTTGCGGTGTTCT | VQTTQEEDGC |
| | | TCTCTTGTCCCTTGTTATAACTCTTTATTG | SCRFPEEEEGG |
| | | TAAGCGCGGAAGGAAGAAATTGTTATAT | CELRVKFSRSA |
| | | ATTTTCAAACAACCTTTTATGCGACCCGT | DAPAYQQGQN |
| | | ACAAACAACTCAGGAAGAGGACGGGTG | QLYNELNLGR |
| | | TTCTTGTCGGTTTCCAGAAGAGGAAGAG | REEYDVLDKR |
| | | GGTGGGTGTGAACTCCGGGTCAAATTTA | RGRDPEMGGK |
| | | GTAGGTCAGCAGATGCGCCGGCGTACCA | PRRKNPQEGL |
| | | ACAAGGCCAAAACCAACTGTATAATGAA | YNELQKDKM |
| | | CTCAATCTCGGTAGGCGTGAGGAATATG | AEAYSEIGMK |
| | | ATGTCCTTGATAAAAGGCGCGGGAGAGA | GERRRGKGHD |
| | | TCCAGAAATGGGCGGAAAACCACGGCG | GLYQGLSTAT |
| | | AAAGAATCCGCAGGAAGGGTTATATAAC | KDTYDALHM |
| | | GAACTTCAAAAGGATAAAATGGCTGAA | QALPPR (SEQ |
| | | GCTTATTCCGAAATTGGCATGAAAGGAG | ID NO: 119) |
| | | AGCGACGTAGGGGCAAAGGGCATGATG | |
| | | GCCTTTACCAAGGGCTCTCAACCGCTAC | |
| | | AAAAGATACTTACGACGCTTTACATATGC | |
| | | AAGCACTTCCACCCAGG (SEQ ID NO: | |
| | | 103) | |
| Construct | CCCCCCTCCCCCCCTTCC | ATGGCACTCCCGGTAACCGCCTTATTGCT | MALPVTALLL |
| C | CTTCCCTTTGCAACGCAA | TCCCCTTGCCCTCTTGCTCCACGCAGCA | PLALLLHAAR |
| | CAATTGTAAGTGCCCTCA | CGCCCCGATATAGTCTTGACTCAATCCCC | PDIVLTQSPPS |
| | CCTGTCAATTGGGACCAC | ACCCAGTTTGGCAATGTCATTAGGCAAA | LAMSLGKRAT |
| | CACTTTCAGTGACCCCAT | CGAGCAACAATTTCATGTAGGGCATCCG | ISCRASESVTIL |
| | GCGAAGTGCTGAGAGAA | AAAGTGTAACGATTTTGGGGAGTCATTT | GSHLIHWYQQ |
| | AGGAAGCTTTCTTACCCT | AATTCATTGGTACCAACAAAAGCCTGGA | KPGQPPTLLIQ |
| | TCATTTGTGAACCCACTG | CAACCCCCGACGCTCTTGATCCAATTAG | LASNVQTGVP |
| | GTCTAAGCCGCTTGGAAT | CATCTAACGTCCAAACCGGAGTCCCCGC | ARFSGSGSRTD |
| | ACGATGAGTGGAAAAGTT | ACGATTCTCAGGATCCGGTTCCCGGACT | FTLTIDPVEED |
| | CATTCTTAATGGAGTGAA | GATTTTACATTAACTATTGATCCGGTAGA | DVAVYYCLQS |
| | ACATGCTTAAATTTCCAG | GGAAGATGACGTCGCTGTCTATTATTGTC | RTIPRTFGGGT |
| | CTCGTGCTGGTCTTTCCA | TTCAAAGTAGGACGATTCCACGGACATT | KLEIKGSTSGS |
| | GTACGGGGCGGCCCTGTC | CGGTGGCGGAACTAAATTGGAGATTAAA | GKPGSGEGST |
| | TGGCCGTAATTCTTCAGA | GGTTCCACCTCTGGTAGTGGGAAACCCG | KGQIQLVQSGP |
| | GTGTCACGCCACACTTGT | GGTCCGGTGAAGGGTCCACTAAAGGCC | ELKKPGETVKI |
| | GGATCTCACGTGCCACAT | AAATTCAACTCGTTCAATCCGGACCAGA | SCKASGYTFT |
| | GACAGCGCTACAGCTGG | ACTGAAGAAGCCAGGAGAAACTGTCAA | DYSINWVKRA |
| | AACTGGGTGCTTGGTGCC | AATAAGCTGTAAAGCTTCCGGTTATACAT | PGKGLKWMG |
| | CATGGAGTAACAGCGAAA | TTACAGATTATTCCATAAATTGGGTGAAA | WINTETREPAY |
| | AGTGTTAGATCAAGCCTT | AGGGCGCCAGGAAAAGGGTTAAAGTGG | AYDFRGRFAFS |
| | GCTTGGGCTATGAGCCTG | ATGGGTTGGATTAATACAGAGACTCGGG | LETSASTAYLQ |
| | CGGAACAACAACTGGTA | AACCTGCATATGCTTATGATTTTAGGGGA | INNLKYEDTAT |
| | ACAGTTGCCTCAGGGGCC | AGGTTTGCCTTTTCTCTGGAGACTTCCG | YFCALDYSYA |
| | GAAAGCCACGGTGTTAAC | CTTCAACTGCTTATCTCCAAATTAATAAT | MDYWGQGTS |
| | AGCACCCTCATAGTTTGA | CTTAAATATGAGGACACAGCAACATACT | VTVSSAAATT |
| | TCCACCTCAGGGTGGTGA | TCTGTGCTTTGGACTATAGTTATGCTATG | TPAPRPPTPAP |
| | TGTTTAGCAGTTAGTAGT | GATTACTGGGGACAAGGAACCAGTGTC | TIASQPLSLRP |
| | TGCCAATCTGTGTTCACT | ACTGTAAGTTCCGCTGCTGCGACGACCA | EACRPAAGGA |
| | GAAATCTCGGCATACCGT | CTCCTGCACCGCGACCACCCACTCCTGC | VHTRGLDFAC |
| | GTAGTGTACAGGGGTGAA | CCCTACTATTGCTAGTCAACCACTTAGCT | DIYIWAPLAGT |
| | GGATGCCCAGAAGGTACC | TGCGACCTGAGGCATGTCGGCCCGCGGC | CGVLLLSLVIT |
| | CGTAGGTAACCTTAAGAG | AGGTGGCGCAGTCCACACCAGGGGTTT | LYCKRGRKKL |
| | ACTATGGATCTGATCTGG | AGACTTTGCTTGTGATATTTATATTTGGG | LYIFKQPFMRP |
| | GGCCTTGTCCGGAGTGCT | CACCACTCGCCGGGACTTGCGGTGTTCT | VQTTQEEDGC |
| | TTACACACGGCTCAAGGT | TCTCTTGTCCCTTGTTATAACTCTTTATTG | SCRFPEEEEGG |
| | TAAAAAACGTCTAGCCCC | TAAGCGCGGAAGGAAGAAATTGTTATAT | CELRVKFSRSA |
| | ACAGAGCCCGAGGGATTC | ATTTTCAAACAACCTTTTATGCGACCCGT | DAPAYQQGQN |
| | GGGTTTTCCCTTTAAAAA | ACAAACAACTCAGGAAGAGGACGGGTG | QLYNELNLGR |
| | CCCGACTAGAGCTTATGG | TTCTTGTCGGTTTCCAGAAGAGGAAGAG | REEYDVLDKR |
| | TGACAATTATTGCTGTTCA | GGTGGGTGTGAACTCCGGGTCAAATTTA | RGRDPEMGGK |
| | GACGAACAGTGTAATTGT | GTAGGTCAGCAGATGCGCCGGCGTACCA | PRRKNPQEGL |
| | TGTCTATTCACAGCAGTT | ACAAGGCCAAAACCAACTGTATAATGAA | YNELQKDKM |
| | CTATCAGAGCTTTTCCCA | CTCAATCTCGGTAGGCGTGAGGAATATG | AEAYSEIGMK |
| | CAACGGATCTTCTTGGCA | ATGTCCTTGATAAAAGGCGCGGGAGAGA | GERRRGKGHD |
| | AGCAAATACAGCAGGAGT | TCCAGAAATGGGCGGAAAACCACGGCG | GLYQGLSTAT |
| | CAAT (SEQ ID NO: 8) | AAAGAATCCGCAGGAAGGGTTATATAAC | KDTYDALHM |
| | | GAACTTCAAAAGGATAAAATGGCTGAA | QALPPR (SEQ |
| | | GCTTATTCCGAAATTGGCATGAAAGGAG | ID NO: 119) |
| | | AGCGACGTAGGGGCAAAGGGCATGATG | |
| | | GCCTTTACCAAGGGCTCTCAACCGCTAC | |
| | | AAAAGATACTTACGACGCTTTACATATGC | |
| | | AAGCACTTCCACCCAGG (SEQ ID NO: | |
| | | 103) | |
| Construct | TTTGCTCAGCGTAACTTC | ATGGCACTCCCGGTAACCGCCTTATTGCT | MALPVTALLL |
| D | TCCGGGTTACGTGGAGAC | TCCCCTTGCCCTCTTGCTCCACGCAGCA | PLALLLHAAR |
| | CAAAAGGCTACGGAGAC | CGCCCCGATATAGTCTTGACTCAATCCCC | PDIVLTQSPPS |
| | TCGGGCTACGGCCCTGGA | ACCCAGTTTGGCAATGTCATTAGGCAAA | LAMSLGKRAT |
| | GCACCTAGGTGCTCCTAA | CGAGCAACAATTTCATGTAGGGCATCCG | ISCRASESVTIL |
| | AGACGTTAGAAGTTGTAC | AAAGTGTAACGATTTTGGGGAGTCATTT | GSHLIHWYQQ |
| | AAACTCGCCCAATAGGGC | AATTCATTGGTACCAACAAAAGCCTGGA | KPGQPPTLLIQ |
| | CCCCCAACCAGGGGGGT | CAACCCCCGACGCTCTTGATCCAATTAG | LASNVQTGVP |
| | AGCGGGCAAGCACTTCTG | CATCTAACGTCCAAACCGGAGTCCCCGC | ARFSGSGSRTD |
| | TTTCCCCGGTATGATCTCA | ACGATTCTCAGGATCCGGTTCCCGGACT | FTLTIDPVEED |
| | TAGGCTGTACCCACGGCT | GATTTTACATTAACTATTGATCCGGTAGA | DVAVYYCLQS |
| | GAAAGAGAGATTATCGTT | GGAAGATGACGTCGCTGTCTATTATTGTC | RTIPRTFGGGT |
| | ACCCGCCTCACTACTTCG | TTCAAAGTAGGACGATTCCACGGACATT | KLEIKGSTSGS |
| | AGAAGCCCAGTAATGGTT | CGGTGGCGGAACTAAATTGGAGATTAAA | GKPGSGEGST |
| | CATGAAGTTGATCTCGTT | GGTTCCACCTCTGGTAGTGGGAAACCCG | KGQIQLVQSGP |
| | GACCCGGTGTTTCCCCCA | GGTCCGGTGAAGGGTCCACTAAAGGCC | ELKKPGETVKI |
| | CACCAGAAACCTGTGATG | AAATTCAACTCGTTCAATCCGGACCAGA | SCKASGYTFT |
| | GGGGTGGTCATCCCGGTC | ACTGAAGAAGCCAGGAGAAACTGTCAA | DYSINWVKRA |
| | ATGGCGACATGACGGACC | AATAAGCTGTAAAGCTTCCGGTTATACAT | PGKGLKWMG |
| | TCCCCGCGCCGGCACAGG | TTACAGATTATTCCATAAATTGGGTGAAA | WINTETREPAY |
| | GCCTCTTCGGAGGACGAG | AGGGCGCCAGGAAAAGGGTTAAAGTGG | AYDFRGRFAFS |
| | TGACATGGATTCAACCGT | ATGGGTTGGATTAATACAGAGACTCGGG | LETSASTAYLQ |
| | GAAGAGCCTATTGAGCTA | AACCTGCATATGCTTATGATTTTAGGGGA | INNLKYEDTAT |
| | GTGTTGATTCCTCCGCCC | AGGTTTGCCTTTTCTCTGGAGACTTCCG | YFCALDYSYA |
| | CCGTGAATGCGGCTAATC | CTTCAACTGCTTATCTCCAAATTAATAAT | MDYWGQGTS |
| | CCAACTCCGGAGCAGGC | CTTAAATATGAGGACACAGCAACATACT | VTVSSAAATT |
| | GGGCCCAAACCAGGGTC | TCTGTGCTTTGGACTATAGTTATGCTATG | TPAPRPPTPAP |
| | TGGCCTGTCGTAACGCGA | GATTACTGGGGACAAGGAACCAGTGTC | TIASQPLSLRP |
| | AAGTCTGGAGCGGAACC | ACTGTAAGTTCCGCTGCTGCGACGACCA | EACRPAAGGA |
| | GACTACTTTCGGGAAGGC | CTCCTGCACCGCGACCACCCACTCCTGC | VHTRGLDFAC |
| | GTGTTTCCTTTTGTTCCTT | CCCTACTATTGCTAGTCAACCACTTAGCT | DIYIWAPLAGT |
| | TTATCAAGTTTTATGGTGA | TGCGACCTGAGGCATGTCGGCCCGCGGC | CGVLLLSLVIT |
| | CAACTCCTGGTAGACGTT | AGGTGGCGCAGTCCACACCAGGGGTTT | LYCKRGRKKL |
| | TTATTGCGTTTATTGAGAG | AGACTTTGCTTGTGATATTTATATTTGGG | LYIFKQPFMRP |
| | ATTTCCAACAATTGAACA | CACCACTCGCCGGGACTTGCGGTGTTCT | VQTTQEEDGC |
| | GACTAGAACCACTTGTTT | TCTCTTGTCCCTTGTTATAACTCTTTATTG | SCRFPEEEEGG |
| | TATCAAACCCTCACAGAA | TAAGCGCGGAAGGAAGAAATTGTTATAT | CELRVKFSRSA |
| | TAAGATAACA (SEQ ID NO: | ATTTTCAAACAACCTTTTATGCGACCCGT | DAPAYQQGQN |
| | 18) | ACAAACAACTCAGGAAGAGGACGGGTG | QLYNELNLGR |
| | | TTCTTGTCGGTTTCCAGAAGAGGAAGAG | REEYDVLDKR |
| | | GGTGGGTGTGAACTCCGGGTCAAATTTA | RGRDPEMGGK |
| | | GTAGGTCAGCAGATGCGCCGGCGTACCA | PRRKNPQEGL |
| | | ACAAGGCCAAAACCAACTGTATAATGAA | YNELQKDKM |
| | | CTCAATCTCGGTAGGCGTGAGGAATATG | AEAYSEIGMK |
| | | ATGTCCTTGATAAAAGGCGCGGGAGAGA | GERRRGKGHD |
| | | TCCAGAAATGGGCGGAAAACCACGGCG | GLYQGLSTAT |
| | | AAAGAATCCGCAGGAAGGGTTATATAAC | KDTYDALHM |
| | | GAACTTCAAAAGGATAAAATGGCTGAA | QALPPR (SEQ |
| | | GCTTATTCCGAAATTGGCATGAAAGGAG | ID NO: 119) |
| | | AGCGACGTAGGGGCAAAGGGCATGATG | |
| | | GCCTTTACCAAGGGCTCTCAACCGCTAC | |
| | | AAAAGATACTTACGACGCTTTACATATGC | |
| | | AAGCACTTCCACCCAGG (SEQ ID NO: | |
| | | 103) | |
| Construct | ATTCTCGGGCTACGGCCC | ATGGCACTCCCGGTAACCGCCTTATTGCT | MALPVTALLL |
| E | TGGAGCCACTCCGGCTCC | TCCCCTTGCCCTCTTGCTCCACGCAGCA | PLALLLHAAR |
| | TAAAGATTTAGAAGTTTG | CGCCCCGATATAGTCTTGACTCAATCCCC | PDIVLTQSPPS |
| | AGCACACCCGCCCACTAG | ACCCAGTTTGGCAATGTCATTAGGCAAA | LAMSLGKRAT |
| | GGCCCCCCATCCAGGGGG | CGAGCAACAATTTCATGTAGGGCATCCG | ISCRASESVTIL |
| | GCAACGGGCAAGCACTT | AAAGTGTAACGATTTTGGGGAGTCATTT | GSHLIHWYQQ |
| | CTGTTTCCCCGGTATGATC | AATTCATTGGTACCAACAAAAGCCTGGA | KPGQPPTLLIQ |
| | TGATAGGCTGTAACCACG | CAACCCCCGACGCTCTTGATCCAATTAG | LASNVQTGVP |
| | GCTGAAACAGAGATTATC | CATCTAACGTCCAAACCGGAGTCCCCGC | ARFSGSGSRTD |
| | GTTATCCGCTTCACTACTT | ACGATTCTCAGGATCCGGTTCCCGGACT | FTLTIDPVEED |
| | CGAGAAGCCTAGTAATGA | GATTTTACATTAACTATTGATCCGGTAGA | DVAVYYCLQS |
| | TGGGTGAAATTGAATCCG | GGAAGATGACGTCGCTGTCTATTATTGTC | RTIPRTFGGGT |
| | TTGATCCGGTGTCTCCCC | TTCAAAGTAGGACGATTCCACGGACATT | KLEIKGSTSGS |
| | CACACCAGAAACTCATGA | CGGTGGCGGAACTAAATTGGAGATTAAA | GKPGSGEGST |
| | TGAGGGTTGCCATCCCGG | GGTTCCACCTCTGGTAGTGGGAAACCCG | KGQIQLVQSGP |
| | CTACGGCGACGTAGCGGG | GGTCCGGTGAAGGGTCCACTAAAGGCC | ELKKPGETVKI |
| | CATCCCTGCGCTGGCATG | AAATTCAACTCGTTCAATCCGGACCAGA | SCKASGYTFT |
| | AGGCCTCTTAGGAGGACG | ACTGAAGAAGCCAGGAGAAACTGTCAA | DYSINWVKRA |
| | GATGATATGGATCTTGTCG | AATAAGCTGTAAAGCTTCCGGTTATACAT | PGKGLKWMG |
| | TGAAGAGCCTATTGAGCT | TTACAGATTATTCCATAAATTGGGTGAAA | WINTETREPAY |
| | AGTGTCGACTCCTCCGCC | AGGGCGCCAGGAAAAGGGTTAAAGTGG | AYDFRGRFAFS |
| | CCCGTGAATGCGGCTAAT | ATGGGTTGGATTAATACAGAGACTCGGG | LETSASTAYLQ |
| | CCTAACCCCGGAGCAGGT | AACCTGCATATGCTTATGATTTTAGGGGA | INNLKYEDTAT |
| | GGGTCCAATCCAGGGCCT | AGGTTTGCCTTTTCTCTGGAGACTTCCG | YFCALDYSYA |
| | GGCCTGTCGTAATGCGTA | CTTCAACTGCTTATCTCCAAATTAATAAT | MDYWGQGTS |
| | AGTCTGGGACGGAACCG | CTTAAATATGAGGACACAGCAACATACT | VTVSSAAATT |
| | ACTACTTTCGGGAAGGCG | TCTGTGCTTTGGACTATAGTTATGCTATG | TPAPRPPTPAP |
| | TGTTTCCATTTGTTCATTA | GATTACTGGGGACAAGGAACCAGTGTC | TIASQPLSLRP |
| | TTTGTGTGTTTATGGTGAC | ACTGTAAGTTCCGCTGCTGCGACGACCA | EACRPAAGGA |
| | AACTCTGGGTAAACGTTC | CTCCTGCACCGCGACCACCCACTCCTGC | VHTRGLDFAC |
| | TATTGCGTTTATTGAGAGA | CCCTACTATTGCTAGTCAACCACTTAGCT | DIYIWAPLAGT |
| | TTCCCAACAATTGAACAA | TGCGACCTGAGGCATGTCGGCCCGCGGC | CGVLLLSLVIT |
| | ACGAGAACTACCTGTTTT | AGGTGGCGCAGTCCACACCAGGGGTTT | LYCKRGRKKL |
| | ATTAAATTTACACAGAGA | AGACTTTGCTTGTGATATTTATATTTGGG | LYIFKQPFMRP |
| | AGAATTACA (SEQ ID NO: | CACCACTCGCCGGGACTTGCGGTGTTCT | VQTTQEEDGC |
| | 16) | TCTCTTGTCCCTTGTTATAACTCTTTATTG | SCRFPEEEEGG |
| | | TAAGCGCGGAAGGAAGAAATTGTTATAT | CELRVKFSRSA |
| | | ATTTTCAAACAACCTTTTATGCGACCCGT | DAPAYQQGQN |
| | | ACAAACAACTCAGGAAGAGGACGGGTG | QLYNELNLGR |
| | | TTCTTGTCGGTTTCCAGAAGAGGAAGAG | REEYDVLDKR |
| | | GGTGGGTGTGAACTCCGGGTCAAATTTA | RGRDPEMGGK |
| | | GTAGGTCAGCAGATGCGCCGGCGTACCA | PRRKNPQEGL |
| | | ACAAGGCCAAAACCAACTGTATAATGAA | YNELQKDKM |
| | | CTCAATCTCGGTAGGCGTGAGGAATATG | AEAYSEIGMK |
| | | ATGTCCTTGATAAAAGGCGCGGGAGAGA | GERRRGKGHD |
| | | TCCAGAAATGGGCGGAAAACCACGGCG | GLYQGLSTAT |
| | | AAAGAATCCGCAGGAAGGGTTATATAAC | KDTYDALHM |
| | | GAACTTCAAAAGGATAAAATGGCTGAA | QALPPR (SEQ |
| | | GCTTATTCCGAAATTGGCATGAAAGGAG | ID NO: 119) |
| | | AGCGACGTAGGGGCAAAGGGCATGATG | |
| | | GCCTTTACCAAGGGCTCTCAACCGCTAC | |
| | | AAAAGATACTTACGACGCTTTACATATGC | |
| | | AAGCACTTCCACCCAGG (SEQ ID NO: | |
| | | 103) | |
| Construct | GTGGCCACGCCCGGGCC | ATGGCTCTGCCTGTGACAGCTCTGCTGC | MALPVTALLL |
| F | ACCGATACTTCCCTTCAC | TGCCTCTGGCTCTGCTTCTGCATGCCGCC | PLALLLHAAR |
| | TCCTTCGGGACTGTTGGG | AGACCTGACATCCAGATGACCCAGACAA | PDIQMTQTTSS |
| | GAGGAACACAACAGGGC | CCAGCAGCCTGTCTGCCAGCCTGGGCGA | LSASLGDRVTI |
| | TCCCCTGTTTTCCCATTCC | TAGAGTGACCATCAGCTGTAGAGCCAGC | SCRASQDISKY |
| | TTCCCCCTTTTCCCAACC | CAGGACATCAGCAAGTACCTGAACTGGT | LNWYQQKPD |
| | CCAACCGCCGTATCTGGT | ATCAGCAGAAACCCGACGGCACCGTGA | GTVKLLIYHTS |
| | GGCGGCAAGACACACGG | AGCTGCTGATCTACCACACCAGCAGACT | RLHSGVPSRFS |
| | GTCTTTCCCTCTAAAGCA | GCACAGCGGCGTGCCAAGCAGATTTTCT | GSGSGTDYSL |
| | CAATTGTGTGTGTGTCCC | GGCAGCGGCTCTGGCACCGACTACAGC | TISNLEQEDIA |
| | AGGTCCTCCTGCGTACGG | CTGACAATCAGCAACCTGGAACAAGAG | TYFCQQGNTL |
| | TGCGGGAGTGCTCCCACC | GATATCGCTACCTACTTCTGCCAGCAAG | PYTFGGGTKL |
| | CAACTGTTGTAAGCCTGT | GCAACACCCTGCCTTACACCTTTGGCGG | EITGGGGSGG |
| | CCAACGCGTCGTCCTGGC | AGGCACCAAGCTGGAAATCACAGGCGG | GGSGGGGSEV |
| | AAGACTATGACGTCGCAT | CGGAGGAAGCGGAGGCGGAGGATCTGG | KLQESGPGLV |
| | GTTCCGCTGCGGATGCCG | TGGTGGTGGATCTGAAGTGAAACTGCAA | APSQSLSVTCT |
| | ACCGGGTAACCGGTTCCC | GAGTCTGGCCCTGGCCTGGTGGCCCCAT | VSGVSLPDYG |
| | CAGTGTGTGTAGTGCGAT | CTCAATCTCTGAGCGTGACCTGTACCGT | VSWIRQPPRK |
| | CTTCCAGGTCCTCCTGGT | CAGCGGAGTGTCCCTGCCTGATTATGGC | GLEWLGVIWG |
| | TGGCGTTGTCCAGAAACT | GTGTCCTGGATCCGGCAGCCTCCTAGAA | SETTYYNSAL |
| | GCTTCAGGTAAGTGGGGT | AAGGCCTGGAATGGCTGGGCGTGATCTG | KSRLTIIKDNS |
| | GTGCCCAATCCCTACAAA | GGGCAGCGAGACAACCTACTACAACAG | KSQVFLKMNS |
| | GGTTGATTCTTTCACCAC | CGCCCTGAAGTCCCGGCTGACCATCATC | LQTDDTAIYY |
| | CTTAGGAATGCTCCGGAG | AAGGACAACTCCAAGAGCCAGGTGTTC | CAKHYYYGGS |
| | GTACCCCAGCAACAGCTG | CTGAAGATGAACAGCCTGCAGACCGAC | YAMDYWGQG |
| | GGATCTGACCGGAGGCTA | GACACCGCCATCTACTATTGCGCCAAGC | TSVTVSSIEVM |
| | ATTGTCTACGGGTGGTGT | ACTACTACTACGGCGGCAGCTACGCCAT | YPPPYLDNEK |
| | TTCCTTTTTCTTTTCACAC | GGATTATTGGGGCCAGGGCACCAGCGTG | SNGTIIHVKGK |
| | AACTCTACTGCTGACAAC | ACCGTGTCTAGCATCGAAGTGATGTACC | HLCPSPLFPGP |
| | TCACTGACTATCCACTTG | CTCCACCTTACCTGGACAACGAGAAGTC | SKPFWVLVVV |
| | CTCTGTCACG (SEQ ID NO: | CAACGGCACCATCATCCACGTGAAGGGC | GGVLACYSLL |
| | 17) | AAGCACCTGTGTCCTTCTCCACTGTTCC | VTVAFIIFWVR |
| | | CCGGACCTAGCAAGCCTTTCTGGGTGCT | SKRSRLLHSD |
| | | CGTTGTTGTTGGCGGCGTGCTGGCCTGT | YMNMTPRRPG |
| | | TACTCTCTGCTGGTTACCGTGGCCTTCAT | PTRKHYQPYA |
| | | CATCTTTTGGGTCCGAAGCAAGCGGAGC | PPRDFAAYRSR |
| | | CGGCTGCTGCACTCCGACTACATGAACA | VKFSRSADAP |
| | | TGACCCCTAGACGGCCCGGACCAACCA | AYQQGQNQLY |
| | | GAAAGCACTACCAGCCTTACGCTCCTCC | NELNLGRREE |
| | | TAGAGACTTCGCCGCCTACCGGTCCAGA | YDVLDKRRGR |
| | | GTGAAGTTCAGCAGATCCGCCGATGCTC | DPEMGGKPRR |
| | | CCGCCTATCAGCAGGGCCAAAACCAGCT | KNPQEGLYNE |
| | | GTACAACGAGCTGAACCTGGGGAGAAG | LQKDKMAEA |
| | | AGAAGAGTACGACGTGCTGGACAAGCG | YSEIGMKGER |
| | | GAGAGGCAGAGATCCTGAAATGGGCGG | RRGKGHDGLY |
| | | CAAGCCCAGACGGAAGAATCCTCAAGA | QGLSTATKDT |
| | | GGGCCTGTATAATGAGCTGCAGAAAGAC | YDALHMQALP |
| | | AAGATGGCCGAGGCCTACAGCGAGATC | PR (SEQ ID NO: |
| | | GGAATGAAGGGCGAGCGCAGAAGAGGC | 29) |
| | | AAGGGACACGATGGACTGTACCAGGGC | |
| | | CTGAGCACCGCCACCAAGGATACCTATG | |
| | | ATGCCCTGCACATGCAGGCCCTGCCTCC | |
| | | AAGA (SEQ ID NO: 21) | |
| Construct | CCCCCCTCCCCCCCTTCC | ATGGCCCTCCCCGTCACAGCTCTCCTGC | MALPVTALLL |
| G | CTTCCCTTTGCAACGCAA | TCCCACTGGCCCTTCTTTTGCACGCTGCT | PLALLLHAAR |
| | CAATTGTAAGTGCCCTCA | CGCCCCGATATCGTGCTCACCCAGTCAC | PDIVLTQSPPS |
| | CCTGTCAATTGGGACCAC | CTCCAAGCCTTGCCATGAGCCTCGGGAA | LAMSLGKRAT |
| | CACTTTCAGTGACCCCAT | ACGGGCTACCATCTCCTGCCGGGCTTCA | ISCRASESVTIL |
| | GCGAAGTGCTGAGAGAA | GAGTCCGTCACCATCCTCGGGTCACACC | GSHLIHWYQQ |
| | AGGAAGCTTTCTTACCCT | TCATCCACTGGTACCAACAGAAACCAGG | KPGQPPTLLIQ |
| | TCATTTGTGAACCCACTG | GCAGCCTCCTACCCTCTTGATCCAGTTG | LASNVQTGVP |
| | GTCTAAGCCGCTTGGAAT | GCCTCCAACGTGCAAACTGGGGTTCCCG | ARFSGSGSRTD |
| | ACGATGAGTGGAAAAGTT | CCAGGTTCAGTGGCTCCGGATCCCGGAC | FTLTIDPVEED |
| | CATTCTTAATGGAGTGAA | AGATTTCACACTTACCATCGATCCTGTGG | DVAVYYCLQS |
| | ACATGCTTAAATTTCCAG | AGGAGGACGATGTGGCCGTCTATTACTG | RTIPRTFGGGT |
| | CTCGTGCTGGTCTTTCCA | CCTGCAGTCTCGCACCATCCCTCGGACC | KLEIKGSTSGS |
| | GTACGGGGCGGCCCTGTC | TTCGGTGGAGGCACCAAGCTCGAGATCA | GKPGSGEGST |
| | TGGCCGTAATTCTTCAGA | AGGGTAGCACCTCCGGCTCTGGAAAGC | KGQIQLVQSGP |
| | GTGTCACGCCACACTTGT | CAGGCTCTGGTGAGGGTTCTACCAAGGG | ELKKPGETVKI |
| | GGATCTCACGTGCCACAT | CCAAATCCAGCTGGTCCAGTCTGGGCCC | SCKASGYTFT |
| | GACAGCGCTACAGCTGG | GAGCTGAAGAAACCCGGGGAGACCGTG | DYSINWVKRA |
| | AACTGGGTGCTTGGTGCC | AAGATCTCCTGCAAGGCCTCCGGTTATA | PGKGLKWMG |
| | CATGGAGTAACAGCGAAA | CCTTCACCGACTACTCCATCAACTGGGT | WINTETREPAY |
| | AGTGTTAGATCAAGCCTT | CAAGCGCGCTCCTGGAAAGGGCCTCAA | AYDFRGRFAFS |
| | GCTTGGGCTATGAGCCTG | GTGGATGGGCTGGATCAACACCGAAACC | LETSASTAYLQ |
| | CGGAACAACAACTGGTA | CGCGAGCCTGCCTATGCTTACGACTTCA | INNLKYEDTAT |
| | ACAGTTGCCTCAGGGGCC | GGGGCCGGTTCGCTTTCTCACTGGAGAC | YFCALDYSYA |
| | GAAAGCCACGGTGTTAAC | CTCCGCTTCCACAGCCTACCTCCAGATC | MDYWGQGTS |
| | AGCACCCTCATAGTTTGA | AACAACCTCAAGTACGAAGACACCGCC | VTVSSAAATT |
| | TCCACCTCAGGGTGGTGA | ACCTATTTCTGCGCTCTCGACTATTCCTA | TPAPRPPTPAP |
| | TGTTTAGCAGTTAGTAGT | CGCTATGGACTACTGGGGTCAGGGCACC | TIASQPLSLRP |
| | TGCCAATCTGTGTTCACT | TCTGTGACCGTCTCTAGCGCAGCCGCCA | EACRPAAGGA |
| | GAAATCTCGGCATACCGT | CCACAACACCAGCCCCACGGCCACCTAC | VHTRGLDFAC |
| | GTAGTGTACAGGGGTGAA | TCCCGCACCCACCATCGCATCCCAACCA | DIYIWAPLAGT |
| | GGATGCCCAGAAGGTACC | CTCAGTCTGAGGCCCGAGGCCTGTAGAC | CGVLLLSLVIT |
| | CGTAGGTAACCTTAAGAG | CTGCTGCTGGAGGCGCAGTGCATACCCG | LYCRSKRSRLL |
| | ACTATGGATCTGATCTGG | CGGTCTCGACTTCGCCTGCGACATCTATA | HSDYMNMTP |
| | GGCCTTGTCCGGAGTGCT | TCTGGGCCCCATTGGCAGGTACCTGTGG | RRPGPTRKHY |
| | TTACACACGGCTCAAGGT | CGTGCTGCTGCTGTCACTCGTCATCACC | QPYAPPRDFA |
| | TAAAAAACGTCTAGCCCC | CTGTACTGCCGGAGTAAGCGCTCTAGGC | AYRSRVKFSRS |
| | ACAGAGCCCGAGGGATTC | TGTTGCACAGCGACTACATGAACATGAC | ADAPAYQQGQ |
| | GGGTTTTCCCTTTAAAAA | CCCAAGAAGACCAGGGCCTACCCGGAA | NQLYNELNLG |
| | CCCGACTAGAGCTTATGG | GCACTACCAGCCATACGCACCTCCCCGG | RREEYDVLDK |
| | TGACAATTATTGCTGTTCA | GACTTTGCCGCCTATCGGTCTCGGGTGA | RRGRDPEMGG |
| | GACGAACAGTGTAATTGT | AGTTCTCACGCTCCGCTGATGCCCCAGC | KPRRKNPQEG |
| | TGTCTATTCACAGCAGTT | ATACCAGCAGGGGCAGAACCAGCTGTA | LYNELQKDKM |
| | CTATCAGAGCTTTTCCCA | CAATGAGCTCAACCTCGGTCGCCGCGAA | AEAYSEIGMK |
| | CAACGGATCTTCTTGGCA | GAGTACGACGTGCTCGACAAGAGAAGG | GERRRGKGHD |
| | AGCAAATACAGCAGGAGT | GGCAGGGACCCTGAGATGGGAGGCAAG | GLYQGLSTAT |
| | CAAT (SEQ ID NO: 8) | CCCCGCAGAAAGAATCCCCAGGAAGGT | KDTYDALHM |
| | | CTGTACAACGAGCTGCAAAAGGATAAG | QALPPR (SEQ |
| | | ATGGCTGAGGCCTACAGCGAGATCGGCA | ID NO: 120) |
| | | TGAAGGGCGAAAGGAGACGGGGAAAG | |
| | | GGCCACGACGGGCTCTACCAGGGACTCT | |
| | | CCACCGCCACCAAGGACACCTACGACG | |
| | | CCCTCCACATGCAGGCTCTGCCACCCAG | |
| | | G (SEQ ID NO: 104) | |
| Construct | CCCCCCTCCCCCCCTTCC | ATGGCACTTCCCGTCACCGCTCTCCTGC | MALPVTALLL |
| H | CTTCCCTTTGCAACGCAA | TGCCCCTCGCACTGCTGCTCCATGCAGC | PLALLLHAAR |
| | CAATTGTAAGTGCCCTCA | CCGCCCAGACATCGTCCTGACCCAGTCC | PDIVLTQSPPS |
| | CCTGTCAATTGGGACCAC | CCTCCCTCCCTCGCAATGTCCCTCGGGA | LAMSLGKRAT |
| | CACTTTCAGTGACCCCAT | AACGGGCCACCATCAGCTGCCGGGCCTC | ISCRASESVTIL |
| | GCGAAGTGCTGAGAGAA | TGAGTCAGTGACAATCCTCGGAAGCCAT | GSHLIHWYQQ |
| | AGGAAGCTTTCTTACCCT | CTGATCCATTGGTACCAGCAGAAACCCG | KPGQPPTLLIQ |
| | TCATTTGTGAACCCACTG | GTCAGCCTCCAACCCTCCTCATCCAGCT | LASNVQTGVP |
| | GTCTAAGCCGCTTGGAAT | GGCCTCCAACGTGCAGACAGGAGTCCC | ARFSGSGSRTD |
| | ACGATGAGTGGAAAAGTT | CGCTCGGTTCTCAGGCAGCGGTTCCAGG | FTLTIDPVEED |
| | CATTCTTAATGGAGTGAA | ACCGACTTCACCCTGACCATCGACCCCG | DVAVYYCLQS |
| | ACATGCTTAAATTTCCAG | TGGAAGAGGACGATGTGGCTGTGTACTA | RTIPRTFGGGT |
| | CTCGTGCTGGTCTTTCCA | CTGCCTCCAGTCCCGGACCATCCCACGG | KLEIKGSTSGS |
| | GTACGGGGCGGCCCTGTC | ACCTTCGGAGGTGGGACAAAGCTGGAG | GKPGSGEGST |
| | TGGCCGTAATTCTTCAGA | ATCAAAGGCAGCACCAGCGGTTCTGGC | KGQIQLVQSGP |
| | GTGTCACGCCACACTTGT | AAGCCAGGGTCAGGTGAGGGGAGCACA | ELKKPGETVKI |
| | GGATCTCACGTGCCACAT | AAGGGTCAGATCCAGCTGGTGCAGAGC | SCKASGYTFT |
| | GACAGCGCTACAGCTGG | GGTCCCGAGCTGAAGAAGCCCGGGGAG | DYSINWVKRA |
| | AACTGGGTGCTTGGTGCC | ACCGTTAAGATCTCCTGCAAGGCTAGCG | PGKGLKWMG |
| | CATGGAGTAACAGCGAAA | GGTACACCTTCACCGACTATAGTATCAAC | WINTETREPAY |
| | AGTGTTAGATCAAGCCTT | TGGGTCAAGCGCGCTCCTGGCAAGGGG | AYDFRGRFAFS |
| | GCTTGGGCTATGAGCCTG | CTCAAGTGGATGGGGTGGATCAACACCG | LETSASTAYLQ |
| | CGGAACAACAACTGGTA | AAACCAGGGAGCCCGCATACGCTTATGA | INNLKYEDTAT |
| | ACAGTTGCCTCAGGGGCC | CTTTCGGGGCCGGTTCGCCTTTTCCCTG | YFCALDYSYA |
| | GAAAGCCACGGTGTTAAC | GAGACCAGCGCCTCTACCGCCTACCTCC | MDYWGQGTS |
| | AGCACCCTCATAGTTTGA | AGATCAACAACCTGAAGTACGAGGACA | VTVSSIEVMYP |
| | TCCACCTCAGGGTGGTGA | CCGCCACCTACTTCTGCGCACTCGACTA | PPYLDNEKSN |
| | TGTTTAGCAGTTAGTAGT | CTCCTACGCTATGGACTACTGGGGTCAG | GTIIHVKGKHL |
| | TGCCAATCTGTGTTCACT | GGTACCTCCGTCACCGTCTCCAGCATCG | CPSPLFPGPSK |
| | GAAATCTCGGCATACCGT | AGGTCATGTACCCTCCTCCCTACCTGGA | PFWVLVVVGG |
| | GTAGTGTACAGGGGTGAA | CAACGAGAAGTCCAACGGCACCATCATC | VLACYSLLVT |
| | GGATGCCCAGAAGGTACC | CATGTGAAGGGCAAGCATCTCTGCCCCA | VAFIIFWVRSK |
| | CGTAGGTAACCTTAAGAG | GCCCACTGTTCCCCGGACCCTCTAAGCC | RSRLLHSDYM |
| | ACTATGGATCTGATCTGG | CTTCTGGGTCCTGGTCGTCGTCGGCGGT | NMTPRRPGPT |
| | GGCCTTGTCCGGAGTGCT | GTTCTGGCTTGCTACAGCTTGCTGGTCA | RKHYQPYAPP |
| | TTACACACGGCTCAAGGT | CCGTCGCCTTCATCATCTTCTGGGTGCGC | RDFAAYRSRV |
| | TAAAAAACGTCTAGCCCC | TCCAAGAGGAGCCGGCTGCTGCATAGCG | KFSRSADAPA |
| | ACAGAGCCCGAGGGATTC | ACTACATGAACATGACCCCTAGAAGGCC | YQQGQNQLYN |
| | GGGTTTTCCCTTTAAAAA | TGGTCCAACCCGCAAGCACTACCAGCCT | ELNLGRREEY |
| | CCCGACTAGAGCTTATGG | TACGCCCCTCCACGGGACTTCGCAGCCT | DVLDKRRGRD |
| | TGACAATTATTGCTGTTCA | ACCGGTCACGGGTGAAGTTCTCTCGGAG | PEMGGKPRRK |
| | GACGAACAGTGTAATTGT | CGCAGATGCCCCAGCATACCAGCAGGGC | NPQEGLYNEL |
| | TGTCTATTCACAGCAGTT | CAGAACCAGCTGTACAACGAACTTAACC | QKDKMAEAYS |
| | CTATCAGAGCTTTTCCCA | TTGGTCGGCGGGAGGAATACGATGTGCT | EIGMKGERRR |
| | CAACGGATCTTCTTGGCA | GGACAAGCGCAGGGGTCGGGATCCTGA | GKGHDGLYQG |
| | AGCAAATACAGCAGGAGT | AATGGGGGGAAACCACGCCGGAAGAA | LSTATKDTYD |
| | CAAT (SEQ ID NO: 8) | CCCACAGGAGGGGCTCTATAACGAGCTC | ALHMQALPPR |
| | | CAAAAGGATAAGATGGCTGAGGCTTACA | (SEQ ID NO: |
| | | GCGAGATTGGAATGAAGGGAGAAAGAA | 121) |
| | | GACGGGGCAAGGGTCACGACGGGTTGT | |
| | | ACCAGGGTCTGAGCACCGCCACCAAGG | |
| | | ACACCTACGACGCCCTCCACATGCAAGC | |
| | | CCTTCCACCCCGC (SEQ ID NO: 105) | |
| Construct | CCCCCCTCCCCCCCTTCC | ATGGCACTCCCCGTTACCGCCCTTCTGCT | MALPVTALLL |
| I | CTTCCCTTTGCAACGCAA | GCCTCTCGCCTTGCTGCTGCACGCAGCC | PLALLLHAAR |
| | CAATTGTAAGTGCCCTCA | AGACCACAGGTCAAGCTGGAGGAGTCT | PQVKLEESGG |
| | CCTGTCAATTGGGACCAC | GGTGGCGGGCTCGTTCAAGCAGGTCGG | GLVQAGRSLR |
| | CACTTTCAGTGACCCCAT | AGTCTCCGCCTGTCTTGCGCAGCATCAG | LSCAASEHTFS |
| | GCGAAGTGCTGAGAGAA | AGCATACCTTCTCCTCACACGTGATGGG | SHVMGWFRQ |
| | AGGAAGCTTTCTTACCCT | GTGGTTCAGGCAAGCTCCCGGTAAGGA | APGKERESVA |
| | TCATTTGTGAACCCACTG | GAGGGAGTCCGTGGCCGTTATCGGCTGG | VIGWRDISTSY |
| | GTCTAAGCCGCTTGGAAT | CGCGATATCAGCACCTCCTACGCAGACA | ADSVKGRFTIS |
| | ACGATGAGTGGAAAAGTT | GCGTTAAGGGCCGGTTCACTATCTCCAG | RDNAKKTLYL |
| | CATTCTTAATGGAGTGAA | GGACAACGCTAAGAAGACACTCTACCTC | QMNSLKPEDT |
| | ACATGCTTAAATTTCCAG | CAGATGAACAGTCTGAAGCCCGAGGAC | AVYYCAARRI |
| | CTCGTGCTGGTCTTTCCA | ACCGCAGTGTACTATTGCGCTGCTCGGC | DAADFDSWG |
| | GTACGGGGCGGCCCTGTC | GGATCGATGCTGCCGACTTCGACAGCTG | QGTQVTVSSG |
| | TGGCCGTAATTCTTCAGA | GGGTCAAGGGACCCAGGTCACCGTTTCC | GGGSGGGGSG |
| | GTGTCACGCCACACTTGT | AGCGGAGGTGGCGGAAGTGGTGGCGGA | GGGSEVQLVE |
| | GGATCTCACGTGCCACAT | GGATCAGGTGGTGGAGGCTCCGAGGTC | SGGGLVQAGG |
| | GACAGCGCTACAGCTGG | CAGCTGGTGGAATCAGGAGGCGGCTTG | SLRLSCAASG |
| | AACTGGGTGCTTGGTGCC | GTGCAGGCTGGTGGGTCTTTGCGGTTGT | RTFTMGWFRQ |
| | CATGGAGTAACAGCGAAA | CCTGCGCAGCTTCCGGCAGGACCTTCAC | APGKEREFVA |
| | AGTGTTAGATCAAGCCTT | CATGGGATGGTTCAGACAAGCCCCAGGT | AISLSPTLAYY |
| | GCTTGGGCTATGAGCCTG | AAGGAGCGGGAGTTTGTGGCCGCAATCT | AESVKGRFTIS |
| | CGGAACAACAACTGGTA | CACTGTCTCCCACCCTCGCTTACTACGC | RDNAKNTVVL |
| | ACAGTTGCCTCAGGGGCC | CGAGAGTGTGAAGGGGCGCTTCACAAT | QMNSLKPEDT |
| | GAAAGCCACGGTGTTAAC | CAGTCGCGACAACGCAAAGAACACCGT | ALYYCAADRK |
| | AGCACCCTCATAGTTTGA | CGTCCTGCAAATGAACTCCCTGAAGCCT | SVMSIRPDYW |
| | TCCACCTCAGGGTGGTGA | GAGGATACCGCACTCTATTACTGCGCCG | GQGTQVTVSS |
| | TGTTTAGCAGTTAGTAGT | CCGATCGGAAGAGCGTCATGTCCATCCG | TSTTTPAPRPP |
| | TGCCAATCTGTGTTCACT | GCCCGACTATTGGGGCCAAGGCACCCAA | TPAPTIASQPL |
| | GAAATCTCGGCATACCGT | GTGACCGTCAGCTCCACCTCCACAACCA | SLRPEACRPAA |
| | GTAGTGTACAGGGGTGAA | CTCCCGCCCCAAGACCACCTACCCCAGC | GGAVHTRGLD |
| | GGATGCCCAGAAGGTACC | CCCAACAATCGCATCCCAGCCTCTGTCC | FACDIYIWAPL |
| | CGTAGGTAACCTTAAGAG | CTTCGGCCCGAAGCTTGTCGCCCTGCAG | AGTCGVLLLS |
| | ACTATGGATCTGATCTGG | CAGGTGGAGCAGTGCACACCCGGGGAC | LVITLYCRSKR |
| | GGCCTTGTCCGGAGTGCT | TGGACTTCGCCTGCGACATCTACATCTG | SRLLHSDYMN |
| | TTACACACGGCTCAAGGT | GGCACCCCTGGCTGGAACCTGCGGCGT | MTPRRPGPTR |
| | TAAAAAACGTCTAGCCCC | GTTGCTGCTGAGCCTGGTGATCACCCTC | KHYQPYAPPR |
| | ACAGAGCCCGAGGGATTC | TACTGCCGCTCTAAGAGAAGCCGGCTGC | DFAAYRSRVK |
| | GGGTTTTCCCTTTAAAAA | TGCATAGCGACTACATGAACATGACCCC | FSRSADAPAY |
| | CCCGACTAGAGCTTATGG | TAGGAGACCAGGACCCACCCGGAAGCA | QQGQNQLYNE |
| | TGACAATTATTGCTGTTCA | CTACCAGCCTTACGCTCCTCCACGGGAT | LNLGRREEYD |
| | GACGAACAGTGTAATTGT | TTCGCTGCTTACCGCAGCCGGGTGAAGT | VLDKRRGRDP |
| | TGTCTATTCACAGCAGTT | TTTCCAGGTCAGCTGACGCCCCTGCCTA | EMGGKPRRKN |
| | CTATCAGAGCTTTTCCCA | CCAGCAGGGCCAGAACCAATTGTACAA | PQEGLYNELQ |
| | CAACGGATCTTCTTGGCA | CGAACTGAATCTGGGACGGCGCGAGGA | KDKMAEAYSE |
| | AGCAAATACAGCAGGAGT | ATACGACGTCCTGGACAAGAGGCGGGG | IGMKGERRRG |
| | CAAT (SEQ ID NO: 8) | TAGAGATCCCGAGATGGGCGGGAAACCT | KGHDGLYQGL |
| | | CGGCGGAAGAACCCTCAGGAGGGGCTC | STATKDTYDA |
| | | TACAACGAGCTGCAGAAGGATAAGATG | LHMQALPPR |
| | | GCCGAAGCCTACTCCGAGATCGGGATGA | (SEQ ID NO: |
| | | AGGGTGAACGGAGGAGGGGCAAGGGA | 123) |
| | | CACGACGGCCTGTATCAGGGCCTCAGCA | |
| | | CCGCTACCAAGGACACCTACGACGCCCT | |
| | | GCACATGCAGGCTCTCCCACCACGG | |
| | | (SEQ ID NO: 107) | |
| Construct | CCCCCCTCCCCCCCTTCC | ATGGCTCTTCCCGTCACCGCTTTGCTGCT | MALPVTALLL |
| J | CTTCCCTTTGCAACGCAA | GCCCCTGGCACTCCTCCTCCATGCTGCT | PLALLLHAAR |
| | CAATTGTAAGTGCCCTCA | CGGCCTCAGGTGAAGCTGGAGGAGAGT | PQVKLEESGG |
| | CCTGTCAATTGGGACCAC | GGTGGCGGTCTGGTGCAAGCTGGCAGAT | GLVQAGRSLR |
| | CACTTTCAGTGACCCCAT | CTCTGCGCCTGTCTTGCGCAGCCAGCGA | LSCAASEHTFS |
| | GCGAAGTGCTGAGAGAA | ACACACCTTCTCCTCCCACGTGATGGGG | SHVMGWFRQ |
| | AGGAAGCTTTCTTACCCT | TGGTTTCGGCAGGCACCCGGGAAAGAG | APGKERESVA |
| | TCATTTGTGAACCCACTG | CGCGAGTCCGTCGCAGTCATCGGGTGGC | VIGWRDISTSY |
| | GTCTAAGCCGCTTGGAAT | GGGACATCTCTACCAGCTACGCAGATTC | ADSVKGRFTIS |
| | ACGATGAGTGGAAAAGTT | CGTCAAGGGCCGGTTCACCATTTCCCGG | RDNAKKTLYL |
| | CATTCTTAATGGAGTGAA | GATAACGCTAAGAAGACCCTCTACCTGC | QMNSLKPEDT |
| | ACATGCTTAAATTTCCAG | AAATGAACTCTCTGAAGCCCGAAGACA | AVYYCAARRI |
| | CTCGTGCTGGTCTTTCCA | CCGCCGTCTACTATTGCGCAGCAAGGCG | DAADFDSWG |
| | GTACGGGGCGGCCCTGTC | CATCGACGCTGCCGACTTCGACTCTTGG | QGTQVTVSSG |
| | TGGCCGTAATTCTTCAGA | GGCCAAGGAACCCAGGTCACCGTGTCTT | GGGSGGGGSG |
| | GTGTCACGCCACACTTGT | CCGGAGGAGGAGGCTCCGGTGGTGGAG | GGGSEVQLVE |
| | GGATCTCACGTGCCACAT | GTTCTGGAGGTGGCGGCTCAGAGGTGC | SGGGLVQAGG |
| | GACAGCGCTACAGCTGG | AGCTCGTGGAGAGCGGTGGTGGACTCG | SLRLSCAASG |
| | AACTGGGTGCTTGGTGCC | TTCAGGCAGGCGGCAGTTTGCGGCTGTC | RTFTMGWFRQ |
| | CATGGAGTAACAGCGAAA | CTGTGCAGCCTCCGGTCGCACTTTCACT | APGKEREFVA |
| | AGTGTTAGATCAAGCCTT | ATGGGATGGTTCCGCCAGGCTCCTGGTA | AISLSPTLAYY |
| | GCTTGGGCTATGAGCCTG | AAGAAAGGGAGTTCGTGGCCGCCATCA | AESVKGRFTIS |
| | CGGAACAACAACTGGTA | GTCTGAGCCCCACCCTCGCATACTACGC | RDNAKNTVVL |
| | ACAGTTGCCTCAGGGGCC | CGAGAGCGTGAAGGGTAGGTTCACTATC | QMNSLKPEDT |
| | GAAAGCCACGGTGTTAAC | AGCCGGGACAACGCCAAGAACACCGTG | ALYYCAADRK |
| | AGCACCCTCATAGTTTGA | GTGCTCCAGATGAATTCCCTGAAGCCTG | SVMSIRPDYW |
| | TCCACCTCAGGGTGGTGA | AGGATACCGCCCTCTACTACTGCGCTGC | GQGTQVTVSSI |
| | TGTTTAGCAGTTAGTAGT | CGACCGCAAGAGCGTGATGAGCATCCG | EVMYPPPYLD |
| | TGCCAATCTGTGTTCACT | GCCTGACTATTGGGGTCAGGGGACACAG | NEKSNGTIIHV |
| | GAAATCTCGGCATACCGT | GTGACCGTCAGCAGCATCGAGGTGATGT | KGKHLCPSPLF |
| | GTAGTGTACAGGGGTGAA | ATCCACCACCCTACCTCGACAACGAGAA | PGPSKPFWVL |
| | GGATGCCCAGAAGGTACC | GTCCAACGGCACCATCATCCACGTCAAG | VVVGGVLAC |
| | CGTAGGTAACCTTAAGAG | GGGAAGCACCTCTGCCCTTCCCCTCTGT | YSLLVTVAFIIF |
| | ACTATGGATCTGATCTGG | TCCCTGGCCCCTCAAAGCCCTTCTGGGT | WVRSKRSRLL |
| | GGCCTTGTCCGGAGTGCT | CCTGGTGGTGGTTGGTGGGGTGCTGGCT | HSDYMNMTP |
| | TTACACACGGCTCAAGGT | TGCTACTCCCTGCTCGTGACCGTGGCTT | RRPGPTRKHY |
| | TAAAAAACGTCTAGCCCC | TCATCATCTTCTGGGTTCGGAGCAAACG | QPYAPPRDFA |
| | ACAGAGCCCGAGGGATTC | GTCCAGACTGCTGCACTCCGACTACATG | AYRSRVKFSRS |
| | GGGTTTTCCCTTTAAAAA | AACATGACCCCAAGAAGACCTGGGCCC | ADAPAYQQGQ |
| | CCCGACTAGAGCTTATGG | ACACGGAAGCATTACCAACCCTATGCAC | NQLYNELNLG |
| | TGACAATTATTGCTGTTCA | CACCTCGGGATTTCGCCGCCTACAGATC | RREEYDVLDK |
| | GACGAACAGTGTAATTGT | CCGGGTCAAGTTCTCCAGGTCCGCCGAT | RRGRDPEMGG |
| | TGTCTATTCACAGCAGTT | GCACCAGCCTATCAGCAGGGGCAAAAC | KPRRKNPQEG |
| | CTATCAGAGCTTTTCCCA | CAGCTGTATAATGAGCTGAACCTTGGAC | LYNELQKDKM |
| | CAACGGATCTTCTTGGCA | GGCGCGAGGAGTACGACGTGCTCGACA | AEAYSEIGMK |
| | AGCAAATACAGCAGGAGT | AAAGACGCGGTCGCGACCCAGAGATGG | GERRRGKGHD |
| | CAAT (SEQ ID NO: 8) | GCGGCAAGCCTAGACGCAAGAATCCCC | GLYQGLSTAT |
| | | AGGAGGGGCTCTATAACGAGTTGCAGA | KDTYDALHM |
| | | AGGATAAGATGGCCGAGGCCTACAGCG | QALPPR (SEQ |
| | | AGATCGGGATGAAAGGCGAAAGACGGC | ID NO: 124) |
| | | GCGGAAAGGGTCACGACGGACTCTACC | |
| | | AGGGCCTGAGCACAGCCACCAAAGACA | |
| | | CCTACGACGCTCTGCATATGCAAGCACT | |
| | | GCCTCCCCGG (SEQ ID NO: 108) | |
| Construct | GTGGCCACGCCCGGGCC | ATGGCTCTGCCTGTGACAGCTCTGCTGC | MALPVTALLL |
| K | ACCGATACTTCCCTTCAC | TGCCTCTGGCTCTGCTTCTGCATGCCGCC | PLALLLHAAR |
| | TCCTTCGGGACTGTTGGG | AGACCTGACATCCAGATGACTCAGAGCC | PDIQMTQSPSS |
| | GAGGAACACAACAGGGC | CCAGCAGCCTGTCTGCCTCTGTGGGAGA | LSASVGDRVTI |
| | TCCCCTGTTTTCCCATTCC | CAGAGTGACAATTACCTGCCGGGCCAGC | TCRASQDVNT |
| | TTCCCCCTTTTCCCAACC | CAGGATGTGAATACTGCTGTCGCCTGGT | AVAWYQQKPG |
| | CCAACCGCCGTATCTGGT | ATCAACAAAAGCCTGGCAAGGCCCCTA | KAPKLLIYSAS |
| | GGCGGCAAGACACACGG | AGCTCCTGATCTACAGCGCCAGCTTTCT | FLYSGVPSRFS |
| | GTCTTTCCCTCTAAAGCA | GTACAGCGGCGTGCCCAGCAGATTCTCC | GSRSGTDFTLT |
| | CAATTGTGTGTGTGTCCC | GGAAGCAGAAGCGGCACAGATTTCACA | ISSLQPEDFAT |
| | AGGTCCTCCTGCGTACGG | CTGACCATAAGCAGCCTGCAGCCAGAG | YYCQQHYTTP |
| | TGCGGGAGTGCTCCCACC | GATTTCGCCACCTACTATTGCCAGCAGC | PTFGQGTKVEI |
| | CAACTGTTGTAAGCCTGT | ACTACACCACACCTCCAACCTTTGGCCA | KRTGSTSGSG |
| | CCAACGCGTCGTCCTGGC | GGGCACCAAGGTCGAGATTAAGAGAAC | KPGSGEGSEV |
| | AAGACTATGACGTCGCAT | AGGCAGCACATCTGGCTCTGGCAAACCT | QLVESGGGLV |
| | GTTCCGCTGCGGATGCCG | GGATCTGGCGAGGGCTCTGAAGTCCAGC | QPGGSLRLSC |
| | ACCGGGTAACCGGTTCCC | TGGTGGAATCTGGCGGAGGACTGGTTCA | AASGFNIKDT |
| | CAGTGTGTGTAGTGCGAT | ACCTGGCGGCTCTCTGAGACTGTCTTGT | YIHWVRQAPG |
| | CTTCCAGGTCCTCCTGGT | GCCGCCTCCGGCTTCAACATCAAGGACA | KGLEWVARIY |
| | TGGCGTTGTCCAGAAACT | CCTACATCCACTGGGTCCGACAAGCCCC | PTNGYTRYAD |
| | GCTTCAGGTAAGTGGGGT | AGGCAAAGGACTTGAGTGGGTCGCCAG | SVKGRFTISAD |
| | GTGCCCAATCCCTACAAA | GATCTACCCCACCAACGGCTACACCAGA | TSKNTAYLQM |
| | GGTTGATTCTTTCACCAC | TACGCCGACTCTGTGAAGGGCAGATTCA | NSLRAEDTAV |
| | CTTAGGAATGCTCCGGAG | CCATCTCTGCCGACACCAGCAAGAATAC | YYCSRWGGD |
| | GTACCCCAGCAACAGCTG | CGCCTACCTGCAGATGAACTCCCTGAGA | GFYAMDVWG |
| | GGATCTGACCGGAGGCTA | GCCGAAGATACCGCTGTGTATTACTGTTC | QGTLVTVSSIE |
| | ATTGTCTACGGGTGGTGT | CAGATGGGGAGGCGACGGCTTCTACGCC | VMYPPPYLDN |
| | TTCCTTTTTCTTTTCACAC | ATGGATGTTTGGGGCCAAGGCACCCTCG | EKSNGTIIHVK |
| | AACTCTACTGCTGACAAC | TGACCGTTTCTTCTATCGAAGTGATGTAC | GKHLCPSPLFP |
| | TCACTGACTATCCACTTG | CCTCCACCTTACCTGGACAACGAGAAGT | GPSKPFWVLV |
| | CTCTGTCACG (SEQ ID NO: | CCAACGGCACCATCATCCACGTGAAGGG | VVGGVLACYS |
| | 17) | CAAGCACCTGTGTCCTTCTCCACTGTTC | LLVTVAFIIFW |
| | | CCCGGACCTAGCAAGCCTTTCTGGGTGC | VRSKRSRLLH |
| | | TCGTTGTTGTTGGCGGCGTGCTGGCCTG | SDYMNMTPRR |
| | | TTACTCTCTGCTGGTTACCGTGGCCTTCA | PGPTRKHYQP |
| | | TCATCTTTTGGGTCCGAAGCAAGCGGAG | YAPPRDFAAY |
| | | CCGGCTGCTGCACTCCGACTACATGAAC | RSRVKFSRSA |
| | | ATGACCCCTAGACGGCCCGGACCAACCA | DAPAYQQGQN |
| | | GAAAGCACTACCAGCCTTACGCTCCTCC | QLYNELNLGR |
| | | TAGAGACTTCGCCGCCTACCGGTCCAGA | REEYDVLDKR |
| | | GTGAAGTTCAGCAGATCCGCCGATGCTC | RGRDPEMGGK |
| | | CCGCCTATCAGCAGGGCCAAAACCAGCT | PRRKNPQEGL |
| | | GTACAACGAGCTGAACCTGGGGAGAAG | YNELQKDKM |
| | | AGAAGAGTACGACGTGCTGGACAAGCG | AEAYSEIGMK |
| | | GAGAGGCAGAGATCCTGAAATGGGCGG | GERRRGKGHD |
| | | CAAGCCCAGACGGAAGAATCCTCAAGA | GLYQGLSTAT |
| | | GGGCCTGTATAATGAGCTGCAGAAAGAC | KDTYDALHM |
| | | AAGATGGCCGAGGCCTACAGCGAGATC | QALPPR (SEQ |
| | | GGAATGAAGGGCGAGCGCAGAAGAGGC | ID NO: 117) |
| | | AAGGGACACGATGGACTGTACCAGGGC | |
| | | CTGAGCACCGCCACCAAGGATACCTATG | |
| | | ATGCCCTGCACATGCAGGCCCTGCCTCC | |
| | | AAGA (SEQ ID NO: 101) | |
| Construct | GTGGCCACGCCCGGGCC | ATGGCTCTGCCTGTGACAGCTCTGCTGC | MALPVTALLL |
| L | ACCGATACTTCCCTTCAC | TGCCTCTGGCTCTGCTTCTGCATGCCGCC | PLALLLHAAR |
| | TCCTTCGGGACTGTTGGG | AGACCTGACATCCAGATGACTCAGAGCC | PDIQMTQSPSS |
| | GAGGAACACAACAGGGC | CCAGCAGCCTGTCTGCCTCTGTGGGAGA | LSASVGDRVTI |
| | TCCCCTGTTTTCCCATTCC | CAGAGTGACAATTACCTGCCGGGCCAGC | TCRASQDVNT |
| | TTCCCCCTTTTCCCAACC | CAGGATGTGAATACTGCTGTCGCCTGGT | AVAWYQQKPG |
| | CCAACCGCCGTATCTGGT | ATCAACAAAAGCCTGGCAAGGCCCCTA | KAPKLLIYSAS |
| | GGCGGCAAGACACACGG | AGCTCCTGATCTACAGCGCCAGCTTTCT | FLYSGVPSRFS |
| | GTCTTTCCCTCTAAAGCA | GTACAGCGGCGTGCCCAGCAGATTCTCC | GSRSGTDFTLT |
| | CAATTGTGTGTGTGTCCC | GGAAGCAGAAGCGGCACAGATTTCACA | ISSLQPEDFAT |
| | AGGTCCTCCTGCGTACGG | CTGACCATAAGCAGCCTGCAGCCAGAG | YYCQQHYTTP |
| | TGCGGGAGTGCTCCCACC | GATTTCGCCACCTACTATTGCCAGCAGC | PTFGQGTKVEI |
| | CAACTGTTGTAAGCCTGT | ACTACACCACACCTCCAACCTTTGGCCA | KRTGSTSGSG |
| | CCAACGCGTCGTCCTGGC | GGGCACCAAGGTCGAGATTAAGAGAAC | KPGSGEGSEV |
| | AAGACTATGACGTCGCAT | AGGCAGCACATCTGGCTCTGGCAAACCT | QLVESGGGLV |
| | GTTCCGCTGCGGATGCCG | GGATCTGGCGAGGGCTCTGAAGTCCAGC | QPGGSLRLSC |
| | ACCGGGTAACCGGTTCCC | TGGTGGAATCTGGCGGAGGACTGGTTCA | AASGFNIKDT |
| | CAGTGTGTGTAGTGCGAT | ACCTGGCGGCTCTCTGAGACTGTCTTGT | YIHWVRQAPG |
| | CTTCCAGGTCCTCCTGGT | GCCGCCTCCGGCTTCAACATCAAGGACA | KGLEWVARIY |
| | TGGCGTTGTCCAGAAACT | CCTACATCCACTGGGTCCGACAAGCCCC | PTNGYTRYAD |
| | GCTTCAGGTAAGTGGGGT | AGGCAAAGGACTTGAGTGGGTCGCCAG | SVKGRFTISAD |
| | GTGCCCAATCCCTACAAA | GATCTACCCCACCAACGGCTACACCAGA | TSKNTAYLQM |
| | GGTTGATTCTTTCACCAC | TACGCCGACTCTGTGAAGGGCAGATTCA | NSLRAEDTAV |
| | CTTAGGAATGCTCCGGAG | CCATCTCTGCCGACACCAGCAAGAATAC | YYCSRWGGD |
| | GTACCCCAGCAACAGCTG | CGCCTACCTGCAGATGAACTCCCTGAGA | GFYAMDVWG |
| | GGATCTGACCGGAGGCTA | GCCGAAGATACCGCTGTGTATTACTGTTC | QGTLVTVSSTT |
| | ATTGTCTACGGGTGGTGT | CAGATGGGGAGGCGACGGCTTCTACGCC | TPAPRPPTPAP |
| | TTCCTTTTTCTTTTCACAC | ATGGATGTTTGGGGCCAAGGCACCCTCG | TIASQPLSLRP |
| | AACTCTACTGCTGACAAC | TGACCGTTTCTTCTACCACCACACCAGC | EACRPAAGGA |
| | TCACTGACTATCCACTTG | TCCTCGGCCTCCAACTCCTGCTCCTACA | VHTRGLDFAC |
| | CTCTGTCACG (SEQ ID NO: | ATTGCCAGCCAGCCTCTGTCTCTGAGGC | DIYIWAPLAGT |
| | 17) | CCGAAGCTTGTAGACCTGCTGCTGGCGG | CGVLLLSLVIT |
| | | AGCCGTGCATACAAGAGGACTGGATTTC | LYCKRGRKKL |
| | | GCCTGCGACATCTACATCTGGGCTCCTCT | LYIFKQPFMRP |
| | | GGCCGGAACATGTGGCGTTCTGCTGCTG | VQTTQEEDGC |
| | | AGCCTGGTCATCACCCTGTACTGTAAGC | SCRFPEEEEGG |
| | | GGGGCAGAAAGAAGCTGCTGTACATCTT | CELRVKFSRSA |
| | | CAAGCAGCCCTTCATGCGGCCCGTGCAG | DAPAYQQGQN |
| | | ACCACACAAGAGGAAGATGGCTGCTCC | QLYNELNLGR |
| | | TGCAGATTCCCCGAGGAAGAAGAAGGC | REEYDVLDKR |
| | | GGCTGCGAGCTGAGAGTGAAGTTCAGC | RGRDPEMGGK |
| | | AGATCCGCCGATGCTCCCGCCTATCAGC | PRRKNPQEGL |
| | | AGGGCCAAAACCAGCTGTACAACGAGC | YNELQKDKM |
| | | TGAACCTGGGGAGAAGAGAAGAGTACG | AEAYSEIGMK |
| | | ACGTGCTGGACAAGCGGAGAGGCAGAG | GERRRGKGHD |
| | | ATCCTGAAATGGGCGGCAAGCCCAGAC | GLYQGLSTAT |
| | | GGAAGAATCCTCAAGAGGGCCTGTATAA | KDTYDALHM |
| | | TGAGCTGCAGAAAGACAAGATGGCCGA | QALPPR (SEQ |
| | | GGCCTACAGCGAGATCGGAATGAAGGG | ID NO: 118) |
| | | CGAGCGCAGAAGAGGCAAGGGACACGA | |
| | | TGGACTGTACCAGGGCCTGAGCACCGCC | |
| | | ACCAAGGATACCTATGATGCCCTGCACA | |
| | | TGCAGGCCCTGCCTCCAAGA (SEQ ID | |
| | | NO: 102) | |
| Construct | CCCCCCTCCCCCCCTTCC | ATGGCACTGCCCGTCACCGCACTCCTGC | MALPVTALLL |
| M | CTTCCCTTTGCAACGCAA | TCCCACTGGCACTGCTGCTCCATGCAGC | PLALLLHAAR |
| | CAATTGTAAGTGCCCTCA | TCGCCCCGATATCCAGATGACCCAGACC | PDIQMTQTTSS |
| | CCTGTCAATTGGGACCAC | ACCTCTAGCCTCAGCGCCTCTCTGGGTG | LSASLGDRVTI |
| | CACTTTCAGTGACCCCAT | ACCGCGTCACCATCTCTTGCCGGGCCAG | SCRASQDISKY |
| | GCGAAGTGCTGAGAGAA | CCAAGACATCTCTAAGTACCTGAACTGG | LNWYQQKPD |
| | AGGAAGCTTTCTTACCCT | TACCAGCAGAAACCTGACGGAACCGTG | GTVKLLIYHTS |
| | TCATTTGTGAACCCACTG | AAGCTGCTGATCTACCACACCAGTCGGC | RLHSGVPSRFS |
| | GTCTAAGCCGCTTGGAAT | TGCATTCCGGGGTGCCTTCCAGGTTCAG | GSGSGTDYSL |
| | ACGATGAGTGGAAAAGTT | CGGTTCCGGCTCTGGGACCGATTATAGT | TISNLEQEDIA |
| | CATTCTTAATGGAGTGAA | CTCACCATCTCCAACCTCGAGCAGGAGG | TYFCQQGNTL |
| | ACATGCTTAAATTTCCAG | ACATCGCAACCTACTTCTGCCAGCAGGG | PYTFGGGTKL |
| | CTCGTGCTGGTCTTTCCA | GAACACCCTGCCCTACACCTTCGGTGGC | EITGGGGSGG |
| | GTACGGGGCGGCCCTGTC | GGGACCAAGCTGGAGATCACTGGAGGT | GGSGGGGSEV |
| | TGGCCGTAATTCTTCAGA | GGTGGCAGCGGAGGTGGAGGATCAGGT | KLQESGPGLV |
| | GTGTCACGCCACACTTGT | GGAGGCGGTAGCGAGGTGAAGCTGCAG | APSQSLSVTCT |
| | GGATCTCACGTGCCACAT | GAGTCCGGACCTGGTCTGGTGGCCCCAA | VSGVSLPDYG |
| | GACAGCGCTACAGCTGG | GCCAGTCCCTCAGCGTCACCTGCACAGT | VSWIRQPPRK |
| | AACTGGGTGCTTGGTGCC | GTCCGGGGTGTCCCTGCCTGACTACGGT | GLEWLGVIWG |
| | CATGGAGTAACAGCGAAA | GTCTCCTGGATCAGGCAACCACCCCGGA | SETTYYNSAL |
| | AGTGTTAGATCAAGCCTT | AGGGTCTCGAGTGGCTGGGCGTCATCTG | KSRLTIIKDNS |
| | GCTTGGGCTATGAGCCTG | GGGCTCCGAGACCACCTACTACAACAGC | KSQVFLKMNS |
| | CGGAACAACAACTGGTA | GCTCTGAAGTCCCGGCTGACCATCATCA | LQTDDTAIYY |
| | ACAGTTGCCTCAGGGGCC | AAGACAACTCCAAGAGCCAGGTGTTCTT | CAKHYYYGGS |
| | GAAAGCCACGGTGTTAAC | GAAGATGAACTCCCTGCAAACCGATGAC | YAMDYWGQG |
| | AGCACCCTCATAGTTTGA | ACCGCCATCTACTACTGCGCCAAGCACT | TSVTVSSIEVM |
| | TCCACCTCAGGGTGGTGA | ACTACTATGGCGGTAGCTACGCCATGGAT | YPPPYLDNEK |
| | TGTTTAGCAGTTAGTAGT | TATTGGGGTCAGGGCACCAGTGTCACCG | SNGTIIHVKGK |
| | TGCCAATCTGTGTTCACT | TCTCCTCCATCGAGGTGATGTACCCTCCA | HLCPSPLFPGP |
| | GAAATCTCGGCATACCGT | CCCTATCTGGACAACGAGAAGTCCAACG | SKPFWVLVVV |
| | GTAGTGTACAGGGGTGAA | GCACCATCATCCACGTGAAGGGCAAGCA | GGVLACYSLL |
| | GGATGCCCAGAAGGTACC | CCTGTGCCCTAGCCCTCTGTTCCCAGGA | VTVAFIIFWVR |
| | CGTAGGTAACCTTAAGAG | CCCTCCAAGCCCTTCTGGGTGCTGGTCG | SKRSRLLHSD |
| | ACTATGGATCTGATCTGG | TGGTGGGAGGAGTCCTGGCCTGCTATTC | YMNMTPRRPG |
| | GGCCTTGTCCGGAGTGCT | CCTCCTCGTCACCGTGGCATTTATCATCT | PTRKHYQPYA |
| | TTACACACGGCTCAAGGT | TCTGGGTCCGGAGCAAGCGGTCACGCCT | PPRDFAAYRSR |
| | TAAAAAACGTCTAGCCCC | GCTCCACTCCGACTACATGAACATGACT | VKFSRSADAP |
| | ACAGAGCCCGAGGGATTC | CCTCGCAGACCTGGACCCACCCGGAAG | AYQQGQNQLY |
| | GGGTTTTCCCTTTAAAAA | CACTACCAGCCTTATGCCCCACCCCGCG | NELNLGRREE |
| | CCCGACTAGAGCTTATGG | ACTTTGCCGCTTACCGCTCTCGGGTCAA | YDVLDKRRGR |
| | TGACAATTATTGCTGTTCA | GTTCTCTCGGTCAGCAGACGCCCCTGCA | DPEMGGKPRR |
| | GACGAACAGTGTAATTGT | TACCAGCAGGGCCAGAACCAGCTGTATA | KNPQEGLYNE |
| | TGTCTATTCACAGCAGTT | ACGAGCTGAACCTCGGCAGACGGGAGG | LQKDKMAEA |
| | CTATCAGAGCTTTTCCCA | AGTACGATGTGCTGGACAAGAGGAGAG | YSEIGMKGER |
| | CAACGGATCTTCTTGGCA | GCAGAGACCCCGAGATGGGTGGTAAGC | RRGKGHDGLY |
| | AGCAAATACAGCAGGAGT | CACGGCGCAAGAACCCACAGGAGGGCT | QGLSTATKDT |
| | CAAT (SEQ ID NO: 8) | TGTACAACGAACTGCAGAAGGACAAGA | YDALHMQALP |
| | | TGGCCGAGGCCTACAGCGAGATCGGCAT | PR (SEQ ID NO: |
| | | GAAGGGAGAGAGGCGCAGGGGCAAGG | 29) |
| | | GTCACGACGGCCTGTACCAAGGGCTGTC | |
| | | CACCGCAACCAAGGACACCTACGATGCC | |
| | | CTGCACATGCAGGCCCTCCCACCAAGG | |
| | | (SEQ ID NO: 19) | |
| Construct | ATTCTCGGGCTACGGCCC | ATGGCTCTGCCTGTGACAGCTCTGCTGC | MALPVTALLL |
| N | TGGAGCCACTCCGGCTCC | TGCCTCTGGCTCTGCTTCTGCATGCCGCC | PLALLLHAAR |
| | TAAAGATTTAGAAGTTTG | AGACCTGACATCCAGATGACTCAGAGCC | PDIQMTQSPSS |
| | AGCACACCCGCCCACTAG | CCAGCAGCCTGTCTGCCTCTGTGGGAGA | LSASVGDRVTI |
| | GGCCCCCCATCCAGGGGG | CAGAGTGACAATTACCTGCCGGGCCAGC | TCRASQDVNT |
| | GCAACGGGCAAGCACTT | CAGGATGTGAATACTGCTGTCGCCTGGT | AVAWYQQKPG |
| | CTGTTTCCCCGGTATGATC | ATCAACAAAAGCCTGGCAAGGCCCCTA | KAPKLLIYSAS |
| | TGATAGGCTGTAACCACG | AGCTCCTGATCTACAGCGCCAGCTTTCT | FLYSGVPSRFS |
| | GCTGAAACAGAGATTATC | GTACAGCGGCGTGCCCAGCAGATTCTCC | GSRSGTDFTLT |
| | GTTATCCGCTTCACTACTT | GGAAGCAGAAGCGGCACAGATTTCACA | ISSLQPEDFAT |
| | CGAGAAGCCTAGTAATGA | CTGACCATAAGCAGCCTGCAGCCAGAG | YYCQQHYTTP |
| | TGGGTGAAATTGAATCCG | GATTTCGCCACCTACTATTGCCAGCAGC | PTFGQGTKVEI |
| | TTGATCCGGTGTCTCCCC | ACTACACCACACCTCCAACCTTTGGCCA | KRTGSTSGSG |
| | CACACCAGAAACTCATGA | GGGCACCAAGGTCGAGATTAAGAGAAC | KPGSGEGSEV |
| | TGAGGGTTGCCATCCCGG | AGGCAGCACATCTGGCTCTGGCAAACCT | QLVESGGGLV |
| | CTACGGCGACGTAGCGGG | GGATCTGGCGAGGGCTCTGAAGTCCAGC | QPGGSLRLSC |
| | CATCCCTGCGCTGGCATG | TGGTGGAATCTGGCGGAGGACTGGTTCA | AASGFNIKDT |
| | AGGCCTCTTAGGAGGACG | ACCTGGCGGCTCTCTGAGACTGTCTTGT | YIHWVRQAPG |
| | GATGATATGGATCTTGTCG | GCCGCCTCCGGCTTCAACATCAAGGACA | KGLEWVARIY |
| | TGAAGAGCCTATTGAGCT | CCTACATCCACTGGGTCCGACAAGCCCC | PTNGYTRYAD |
| | AGTGTCGACTCCTCCGCC | AGGCAAAGGACTTGAGTGGGTCGCCAG | SVKGRFTISAD |
| | CCCGTGAATGCGGCTAAT | GATCTACCCCACCAACGGCTACACCAGA | TSKNTAYLQM |
| | CCTAACCCCGGAGCAGGT | TACGCCGACTCTGTGAAGGGCAGATTCA | NSLRAEDTAV |
| | GGGTCCAATCCAGGGCCT | CCATCTCTGCCGACACCAGCAAGAATAC | YYCSRWGGD |
| | GGCCTGTCGTAATGCGTA | CGCCTACCTGCAGATGAACTCCCTGAGA | GFYAMDVWG |
| | AGTCTGGGACGGAACCG | GCCGAAGATACCGCTGTGTATTACTGTTC | QGTLVTVSSIE |
| | ACTACTTTCGGGAAGGCG | CAGATGGGGAGGCGACGGCTTCTACGCC | VMYPPPYLDN |
| | TGTTTCCATTTGTTCATTA | ATGGATGTTTGGGGCCAAGGCACCCTCG | EKSNGTIIHVK |
| | TTTGTGTGTTTATGGTGAC | TGACCGTTTCTTCTATCGAAGTGATGTAC | GKHLCPSPLFP |
| | AACTCTGGGTAAACGTTC | CCTCCACCTTACCTGGACAACGAGAAGT | GPSKPFWVLV |
| | TATTGCGTTTATTGAGAGA | CCAACGGCACCATCATCCACGTGAAGGG | VVGGVLACYS |
| | TTCCCAACAATTGAACAA | CAAGCACCTGTGTCCTTCTCCACTGTTC | LLVTVAFIIFW |
| | ACGAGAACTACCTGTTTT | CCCGGACCTAGCAAGCCTTTCTGGGTGC | VRSKRSRLLH |
| | ATTAAATTTACACAGAGA | TCGTTGTTGTTGGCGGCGTGCTGGCCTG | SDYMNMTPRR |
| | AGAATTACA (SEQ ID NO: | TTACTCTCTGCTGGTTACCGTGGCCTTCA | PGPTRKHYQP |
| | 16) | TCATCTTTTGGGTCCGAAGCAAGCGGAG | YAPPRDFAAY |
| | | CCGGCTGCTGCACTCCGACTACATGAAC | RSRVKFSRSA |
| | | ATGACCCCTAGACGGCCCGGACCAACCA | DAPAYQQGQN |
| | | GAAAGCACTACCAGCCTTACGCTCCTCC | QLYNELNLGR |
| | | TAGAGACTTCGCCGCCTACCGGTCCAGA | REEYDVLDKR |
| | | GTGAAGTTCAGCAGATCCGCCGATGCTC | RGRDPEMGGK |
| | | CCGCCTATCAGCAGGGCCAAAACCAGCT | PRRKNPQEGL |
| | | GTACAACGAGCTGAACCTGGGGAGAAG | YNELQKDKM |
| | | AGAAGAGTACGACGTGCTGGACAAGCG | AEAYSEIGMK |
| | | GAGAGGCAGAGATCCTGAAATGGGCGG | GERRRGKGHD |
| | | CAAGCCCAGACGGAAGAATCCTCAAGA | GLYQGLSTAT |
| | | GGGCCTGTATAATGAGCTGCAGAAAGAC | KDTYDALHM |
| | | AAGATGGCCGAGGCCTACAGCGAGATC | QALPPR (SEQ |
| | | GGAATGAAGGGCGAGCGCAGAAGAGGC | ID NO: 117) |
| | | AAGGGACACGATGGACTGTACCAGGGC | |
| | | CTGAGCACCGCCACCAAGGATACCTATG | |
| | | ATGCCCTGCACATGCAGGCCCTGCCTCC | |
| | | AAGA (SEQ ID NO: 101) | |
| Construct | CCCCCCTCCCCCCCTTCC | ATGGCTCTGCCTGTGACAGCTCTGCTGC | MALPVTALLL |
| O | CTTCCCTTTGCAACGCAA | TGCCTCTGGCTCTGCTTCTGCATGCCGCC | PLALLLHAAR |
| | CAATTGTAAGTGCCCTCA | AGACCTGACATCCAGATGACTCAGAGCC | PDIQMTQSPSS |
| | CCTGTCAATTGGGACCAC | CCAGCAGCCTGTCTGCCTCTGTGGGAGA | LSASVGDRVTI |
| | CACTTTCAGTGACCCCAT | CAGAGTGACAATTACCTGCCGGGCCAGC | TCRASQDVNT |
| | GCGAAGTGCTGAGAGAA | CAGGATGTGAATACTGCTGTCGCCTGGT | AVAWYQQKPG |
| | AGGAAGCTTTCTTACCCT | ATCAACAAAAGCCTGGCAAGGCCCCTA | KAPKLLIYSAS |
| | TCATTTGTGAACCCACTG | AGCTCCTGATCTACAGCGCCAGCTTTCT | FLYSGVPSRFS |
| | GTCTAAGCCGCTTGGAAT | GTACAGCGGCGTGCCCAGCAGATTCTCC | GSRSGTDFTLT |
| | ACGATGAGTGGAAAAGTT | GGAAGCAGAAGCGGCACAGATTTCACA | ISSLQPEDFAT |
| | CATTCTTAATGGAGTGAA | CTGACCATAAGCAGCCTGCAGCCAGAG | YYCQQHYTTP |
| | ACATGCTTAAATTTCCAG | GATTTCGCCACCTACTATTGCCAGCAGC | PTFGQGTKVEI |
| | CTCGTGCTGGTCTTTCCA | ACTACACCACACCTCCAACCTTTGGCCA | KRTGSTSGSG |
| | GTACGGGGCGGCCCTGTC | GGGCACCAAGGTCGAGATTAAGAGAAC | KPGSGEGSEV |
| | TGGCCGTAATTCTTCAGA | AGGCAGCACATCTGGCTCTGGCAAACCT | QLVESGGGLV |
| | GTGTCACGCCACACTTGT | GGATCTGGCGAGGGCTCTGAAGTCCAGC | QPGGSLRLSC |
| | GGATCTCACGTGCCACAT | TGGTGGAATCTGGCGGAGGACTGGTTCA | AASGFNIKDT |
| | GACAGCGCTACAGCTGG | ACCTGGCGGCTCTCTGAGACTGTCTTGT | YIHWVRQAPG |
| | AACTGGGTGCTTGGTGCC | GCCGCCTCCGGCTTCAACATCAAGGACA | KGLEWVARIY |
| | CATGGAGTAACAGCGAAA | CCTACATCCACTGGGTCCGACAAGCCCC | PTNGYTRYAD |
| | AGTGTTAGATCAAGCCTT | AGGCAAAGGACTTGAGTGGGTCGCCAG | SVKGRFTISAD |
| | GCTTGGGCTATGAGCCTG | GATCTACCCCACCAACGGCTACACCAGA | TSKNTAYLQM |
| | CGGAACAACAACTGGTA | TACGCCGACTCTGTGAAGGGCAGATTCA | NSLRAEDTAV |
| | ACAGTTGCCTCAGGGGCC | CCATCTCTGCCGACACCAGCAAGAATAC | YYCSRWGGD |
| | GAAAGCCACGGTGTTAAC | CGCCTACCTGCAGATGAACTCCCTGAGA | GFYAMDVWG |
| | AGCACCCTCATAGTTTGA | GCCGAAGATACCGCTGTGTATTACTGTTC | QGTLVTVSSIE |
| | TCCACCTCAGGGTGGTGA | CAGATGGGGAGGCGACGGCTTCTACGCC | VMYPPPYLDN |
| | TGTTTAGCAGTTAGTAGT | ATGGATGTTTGGGGCCAAGGCACCCTCG | EKSNGTIIHVK |
| | TGCCAATCTGTGTTCACT | TGACCGTTTCTTCTATCGAAGTGATGTAC | GKHLCPSPLFP |
| | GAAATCTCGGCATACCGT | CCTCCACCTTACCTGGACAACGAGAAGT | GPSKPFWVLV |
| | GTAGTGTACAGGGGTGAA | CCAACGGCACCATCATCCACGTGAAGGG | VVGGVLACYS |
| | GGATGCCCAGAAGGTACC | CAAGCACCTGTGTCCTTCTCCACTGTTC | LLVTVAFIIFW |
| | CGTAGGTAACCTTAAGAG | CCCGGACCTAGCAAGCCTTTCTGGGTGC | VRSKRSRLLH |
| | ACTATGGATCTGATCTGG | TCGTTGTTGTTGGCGGCGTGCTGGCCTG | SDYMNMTPRR |
| | GGCCTTGTCCGGAGTGCT | TTACTCTCTGCTGGTTACCGTGGCCTTCA | PGPTRKHYQP |
| | TTACACACGGCTCAAGGT | TCATCTTTTGGGTCCGAAGCAAGCGGAG | YAPPRDFAAY |
| | TAAAAAACGTCTAGCCCC | CCGGCTGCTGCACTCCGACTACATGAAC | RSRVKFSRSA |
| | ACAGAGCCCGAGGGATTC | ATGACCCCTAGACGGCCCGGACCAACCA | DAPAYQQGQN |
| | GGGTTTTCCCTTTAAAAA | GAAAGCACTACCAGCCTTACGCTCCTCC | QLYNELNLGR |
| | CCCGACTAGAGCTTATGG | TAGAGACTTCGCCGCCTACCGGTCCAGA | REEYDVLDKR |
| | TGACAATTATTGCTGTTCA | GTGAAGTTCAGCAGATCCGCCGATGCTC | RGRDPEMGGK |
| | GACGAACAGTGTAATTGT | CCGCCTATCAGCAGGGCCAAAACCAGCT | PRRKNPQEGL |
| | TGTCTATTCACAGCAGTT | GTACAACGAGCTGAACCTGGGGAGAAG | YNELQKDKM |
| | CTATCAGAGCTTTTCCCA | AGAAGAGTACGACGTGCTGGACAAGCG | AEAYSEIGMK |
| | CAACGGATCTTCTTGGCA | GAGAGGCAGAGATCCTGAAATGGGCGG | GERRRGKGHD |
| | AGCAAATACAGCAGGAGT | CAAGCCCAGACGGAAGAATCCTCAAGA | GLYQGLSTAT |
| | CAAT (SEQ ID NO: 8) | GGGCCTGTATAATGAGCTGCAGAAAGAC | KDTYDALHM |
| | | AAGATGGCCGAGGCCTACAGCGAGATC | QALPPR (SEQ |
| | | GGAATGAAGGGCGAGCGCAGAAGAGGC | ID NO: 117) |
| | | AAGGGACACGATGGACTGTACCAGGGC | |
| | | CTGAGCACCGCCACCAAGGATACCTATG | |
| | | ATGCCCTGCACATGCAGGCCCTGCCTCC | |
| | | AAGA (SEQ ID NO: 101) | |
| Construct | TTTGCTCAGCGTAACTTC | ATGGCTCTGCCTGTGACAGCTCTGCTGC | MALPVTALLL |
| P | TCCGGGTTACGTGGAGAC | TGCCTCTGGCTCTGCTTCTGCATGCCGCC | PLALLLHAAR |
| | CAAAAGGCTACGGAGAC | AGACCTGACATCCAGATGACTCAGAGCC | PDIQMTQSPSS |
| | TCGGGCTACGGCCCTGGA | CCAGCAGCCTGTCTGCCTCTGTGGGAGA | LSASVGDRVTI |
| | GCACCTAGGTGCTCCTAA | CAGAGTGACAATTACCTGCCGGGCCAGC | TCRASQDVNT |
| | AGACGTTAGAAGTTGTAC | CAGGATGTGAATACTGCTGTCGCCTGGT | AVAWYQQKPG |
| | AAACTCGCCCAATAGGGC | ATCAACAAAAGCCTGGCAAGGCCCCTA | KAPKLLIYSAS |
| | CCCCCAACCAGGGGGGT | AGCTCCTGATCTACAGCGCCAGCTTTCT | FLYSGVPSRFS |
| | AGCGGGCAAGCACTTCTG | GTACAGCGGCGTGCCCAGCAGATTCTCC | GSRSGTDFTLT |
| | TTTCCCCGGTATGATCTCA | GGAAGCAGAAGCGGCACAGATTTCACA | ISSLQPEDFAT |
| | TAGGCTGTACCCACGGCT | CTGACCATAAGCAGCCTGCAGCCAGAG | YYCQQHYTTP |
| | GAAAGAGAGATTATCGTT | GATTTCGCCACCTACTATTGCCAGCAGC | PTFGQGTKVEI |
| | ACCCGCCTCACTACTTCG | ACTACACCACACCTCCAACCTTTGGCCA | KRTGSTSGSG |
| | AGAAGCCCAGTAATGGTT | GGGCACCAAGGTCGAGATTAAGAGAAC | KPGSGEGSEV |
| | CATGAAGTTGATCTCGTT | AGGCAGCACATCTGGCTCTGGCAAACCT | QLVESGGGLV |
| | GACCCGGTGTTTCCCCCA | GGATCTGGCGAGGGCTCTGAAGTCCAGC | QPGGSLRLSC |
| | CACCAGAAACCTGTGATG | TGGTGGAATCTGGCGGAGGACTGGTTCA | AASGFNIKDT |
| | GGGGTGGTCATCCCGGTC | ACCTGGCGGCTCTCTGAGACTGTCTTGT | YIHWVRQAPG |
| | ATGGCGACATGACGGACC | GCCGCCTCCGGCTTCAACATCAAGGACA | KGLEWVARIY |
| | TCCCCGCGCCGGCACAGG | CCTACATCCACTGGGTCCGACAAGCCCC | PTNGYTRYAD |
| | GCCTCTTCGGAGGACGAG | AGGCAAAGGACTTGAGTGGGTCGCCAG | SVKGRFTISAD |
| | TGACATGGATTCAACCGT | GATCTACCCCACCAACGGCTACACCAGA | TSKNTAYLQM |
| | GAAGAGCCTATTGAGCTA | TACGCCGACTCTGTGAAGGGCAGATTCA | NSLRAEDTAV |
| | GTGTTGATTCCTCCGCCC | CCATCTCTGCCGACACCAGCAAGAATAC | YYCSRWGGD |
| | CCGTGAATGCGGCTAATC | CGCCTACCTGCAGATGAACTCCCTGAGA | GFYAMDVWG |
| | CCAACTCCGGAGCAGGC | GCCGAAGATACCGCTGTGTATTACTGTTC | QGTLVTVSSIE |
| | GGGCCCAAACCAGGGTC | CAGATGGGGAGGCGACGGCTTCTACGCC | VMYPPPYLDN |
| | TGGCCTGTCGTAACGCGA | ATGGATGTTTGGGGCCAAGGCACCCTCG | EKSNGTIIHVK |
| | AAGTCTGGAGCGGAACC | TGACCGTTTCTTCTATCGAAGTGATGTAC | GKHLCPSPLFP |
| | GACTACTTTCGGGAAGGC | CCTCCACCTTACCTGGACAACGAGAAGT | GPSKPFWVLV |
| | GTGTTTCCTTTTGTTCCTT | CCAACGGCACCATCATCCACGTGAAGGG | VVGGVLACYS |
| | TTATCAAGTTTTATGGTGA | CAAGCACCTGTGTCCTTCTCCACTGTTC | LLVTVAFIIFW |
| | CAACTCCTGGTAGACGTT | CCCGGACCTAGCAAGCCTTTCTGGGTGC | VRSKRSRLLH |
| | TTATTGCGTTTATTGAGAG | TCGTTGTTGTTGGCGGCGTGCTGGCCTG | SDYMNMTPRR |
| | ATTTCCAACAATTGAACA | TTACTCTCTGCTGGTTACCGTGGCCTTCA | PGPTRKHYQP |
| | GACTAGAACCACTTGTTT | TCATCTTTTGGGTCCGAAGCAAGCGGAG | YAPPRDFAAY |
| | TATCAAACCCTCACAGAA | CCGGCTGCTGCACTCCGACTACATGAAC | RSRVKFSRSA |
| | TAAGATAACA (SEQ ID NO: | ATGACCCCTAGACGGCCCGGACCAACCA | DAPAYQQGQN |
| | 18) | GAAAGCACTACCAGCCTTACGCTCCTCC | QLYNELNLGR |
| | | TAGAGACTTCGCCGCCTACCGGTCCAGA | REEYDVLDKR |
| | | GTGAAGTTCAGCAGATCCGCCGATGCTC | RGRDPEMGGK |
| | | CCGCCTATCAGCAGGGCCAAAACCAGCT | PRRKNPQEGL |
| | | GTACAACGAGCTGAACCTGGGGAGAAG | YNELQKDKM |
| | | AGAAGAGTACGACGTGCTGGACAAGCG | AEAYSEIGMK |
| | | GAGAGGCAGAGATCCTGAAATGGGCGG | GERRRGKGHD |
| | | CAAGCCCAGACGGAAGAATCCTCAAGA | GLYQGLSTAT |
| | | GGGCCTGTATAATGAGCTGCAGAAAGAC | KDTYDALHM |
| | | AAGATGGCCGAGGCCTACAGCGAGATC | QALPPR (SEQ |
| | | GGAATGAAGGGCGAGCGCAGAAGAGGC | ID NO: 117) |
| | | AAGGGACACGATGGACTGTACCAGGGC | |
| | | CTGAGCACCGCCACCAAGGATACCTATG | |
| | | ATGCCCTGCACATGCAGGCCCTGCCTCC | |
| | | AAGA (SEQ ID NO: 101) | |

### C. Accessory Elements

As described in this disclosure, the circular RNA constructs and related pharmaceutical compositions, linear RNA polynucleotide, and/or DNA templates disclosed herein may further comprise certain accessory elements (also collective referred to herein as “combined accessory element”). In certain embodiments, these accessory elements may be included within the sequences of the circular RNA, linear RNA polynucleotide and/or DNA template for enhancing circularization, translation or both. Accessory elements are sequences, in certain embodiments, that are located with specificity between or within the enhanced intron elements, enhanced exon elements, or core functional element of the respective polynucleotide. As a nonlimiting example, a combined accessory element (e.g., 5′ and 3′) can include an IRES transacting factor region, a miRNA binding site, a restriction site, an RNA editing region, a structural or sequence element, a granule site, a zip code element, an RNA trafficking element, and/or another specialized sequence that enhances promotes circularization and/or translation of the protein encoded within the circular RNA polynucleotide.

In some embodiments, the combined accessory element comprises an IRES transacting factor (ITAF) region. In some embodiments, the IRES transacting factor region modulates the initiation of translation through binding to PCBP1-PCBP4 (polyC binding protein), PABP1 (polyA binding protein), PTB (polyprimidine tract binding), Argonaute protein family, HNRNPK (Heterogeneous nuclear ribonucleoprotein K protein), or La protein. In some embodiments, the IRES transacting factor region comprises a polyA, polyC, polyAC, or polyprimidine track. In some embodiments, the ITAF region is located within the core functional element. In some embodiments, the ITAF region is located within the TIE.

In certain embodiments, the combined accessory element comprises at least one miRNA binding site. In some embodiments the miRNA binding site is located within the 5′ enhanced intron element, 5′ enhanced exon element, core functional element, 3′ enhanced exon element, and/or 3′ enhanced intron element. In some embodiments, the miRNA binding site is located within the spacer within the enhanced intron element or enhanced exon element. In certain embodiments, the miRNA binding site comprises the entire spacer regions. In some embodiments, the 5′ enhanced intron element and 3′ enhanced intron elements each comprise identical miRNA binding sites. In another embodiment, the miRNA binding site of the 5′ enhanced intron element comprises a different, in length or nucleotides, miRNA binding site than the 3′ enhanced intron element. In one embodiment, the 5′ enhanced exon element and 3′ enhanced exon element comprise identical miRNA binding sites. In other embodiments, the 5′ enhanced exon element and 3′ enhanced exon element comprises different, in length or nucleotides, miRNA binding sites. In some embodiments, the miRNA binding sites are located adjacent to each other within the circular RNA construct, linear RNA polynucleotide precursor, and/or DNA template. In certain embodiments, the first nucleotide of one of the miRNA binding sites follows the first nucleotide last nucleotide of the second miRNA binding site.

In some embodiments, the miRNA binding site is located within a translation initiation element (TIE) of a core functional element. In one embodiment, the miRNA binding site is located before, trailing or within an internal ribosome entry site (IRES). In another embodiment, the miRNA binding site is located before, trailing, or within an aptamer complex.

Incorporation of miRNA sequences within a circular RNA molecule can permit tissue-specific expression of a coding sequence within a core functional element. For example, in a circular RNA intended to express a protein in immune cells, miRNA binding sequences resulting in expression suppression in tissues such as the liver or kidney may be desired. Such miRNA binding sequences may be selected based on the cell or tissue expression of miRNAs. The unique sequences defined by the miRNA nomenclature are widely known and accessible to those working in the microRNA field. For example, they can be found in the miRDB public database. As a non-limiting example, one or more miR-122 target sites can be inserted in the circular RNA.

In some embodiments, the miR-122 site can comprise the following sequence:

(SEQ ID NO: 200)
CAAACACCATTGTCACACTCCAA.

### D. Expression Sequences and Payloads

In some embodiments, the circular RNA constructs comprise at least one expression sequence encoding a binding molecule. In certain embodiments, the circular RNA constructs comprise an IRES and at least one expression sequence encoding a therapeutic protein, wherein the IRES is capable of facilitating expression of the protein when delivered in vivo.

In some embodiments, the circular RNA may encode for various therapeutic proteins, cytokines, immune checkpoint inhibitors, agonists, chimeric antigen receptors, inhibitory receptor agonists, one or more T-Cell Receptors, and/or B-cell Receptors that are available in the art. The chimeric proteins may also include, for example, recombinant fusion proteins, chimeric mutant protein, or other fusion proteins. In some embodiments, the circular RNA comprises more than 1 expression sequence, e.g., 2, 3, 4, or 5 expression sequences. In some embodiments, the circular RNA is a bicistronic RNA. In some embodiments, the bicistronic RNA is codon optimized. Exemplary bicistronic circular RNA are described in WO2021/189059A2, which is incorporated by reference herein in its entirety.

In some embodiments, the expression sequence encodes a therapeutic protein. In some embodiments, the therapeutic protein is selected from the proteins listed in the following table.

| | | Exem- | |
|---|---|---|---|
| | | plary | |
| | | target | |
| Pay- | | cell/ | |
| load | Exemplary Sequences | organ | Exemplary delivery vehicle |
| CD19 CAR | See, e.g., Tables 2A-2C | T cells | 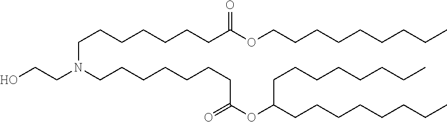 [SMILES: CCCCCCCCCOC(=O)CCCCCCCN(CCO)CCCCCCCC(=O)OC(CCCCCCCC)CCCCCCCC] (50 mol %) DSPC (10 mol %) Beta-sitosterol (28.5% mol %) Cholesterol (10 mol %) PEG DMG (1.5 mol %) |
| BCMA CAR* | See, e.g., Table 2C MALPVTALLLPLALLLHAARPDIVLTQ SPASLAVSLGERATINCRASESVSVIGA HLIHWYQQKPGQPPKLLIYLASNLETG VPARFSGSGSGTDFTLTISSLQAEDAAI YYCLQSRIFPRTFGQGTKLEIKGSTSGS GKPGSGEGSTKGQVQLVQSGSELKKP GASVKVSCKASGYTFTDYSINWVRQA PGQGLEWMGWINTETREPAYAYDFRG RFVFSLDTSVSTAYLQISSLKAEDTAV YYCARDYSYAMDYWGQGTLVTVSSA AATTTPAPRPPTPAPTIASQPLSLRPEA CRPAAGGAVHTRGLDFACDIYIWAPL AGTCGVLLLSLVITLYCKRGRKKLLYI FKQPFMRPVQTTQEEDGCSCRFPEEEE GGCELRVKFSRSADAPAYQQGQNQLY NELNLGRREEYDVLDKRRGRDPEMGG KPRRKNPQEGLYNELQKDKMAEAYSE IGMKGERRRGKGHDGLYQGLSTATKD TYDALHMQALPPR (SEQ ID NO: 201) *The BCMA CAR may be chosen from any of the anti-BCMA CARs disclosed in US Patent Application US 2021/0128618A1 | T cells | 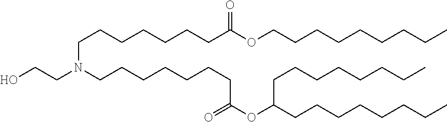 [SMILES: CCCCCCCCCOC(=O)CCCCCCCN(CCO)CCCCCCCC(=O)OC(CCCCCCCC)CCCCCCCC] (50 mol %) DSPC (10 mol %) Beta-sitosterol (28.5% mol %) Cholesterol (10 mol %) PEG DMG (1.5 mol %) |
| MAGE- A4 TCR | TCR alpha chain: KNQVEQSPQSLIILEGKNCTLQCNYTV SPFSNLRWYKQDTGRGPVSLTIMTFSE NTKSNGRYTATLDADTKQSSLHITASQ LSDSASYICVVNHSGGSYIPTFGRGTSL IVHPYIQKPDPAVYQLRDSKSSDKSVC LFTDFDSQTNVSQSKDSDVYITDKTVL DMRSMDFKSNSAVAWSNKSDFACAN AFNNSIIPEDTFFPSPESS (SEQ ID NO: 202) TCR beta chain: DVKVTQSSRYLVKRTGEKVFLECVQD MDHENMFWYRQDPGLGLRLIYFSYDV KMKEKGDIPEGYSVSREKKERFSLILES ASTNQTSMYLCASSFLMTSGDPYEQYF GPGTRLTVTEDLKNVFPPEVAVFEPSE AEISHTOKATLVCLATGFYPDHVELSW WVNGKEVHSGVSTDPQPLKEQPALND SRYCLSSRLRVSATFWQNPRNHFRCQ VQFYGLSENDEWTQDRAKPVTQIVSA EAWGRAD (SEQ ID NO: 203) | T cells | 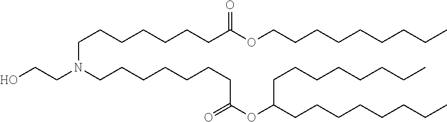 [SMILES: CCCCCCCCCOC(=O)CCCCCCCN(CCO)CCCCCCCC(=O)OC(CCCCCCCC)CCCCCCCC] (50 mol %) DSPC (10 mol %) Beta-sitosterol (28.5% mol %) Cholesterol (10 mol %) PEG DMG (1.5 mol %) |
| NY-ESO TCR | TCRalpha extracellular sequence MQEVTQIPAALSVPEGENLVLNCSFTD SAIYNLQWFRQDPGKGLTSLLLIQSSQ REQTSGRLNASLDKSSGRSTLYIAASQ PGDSATYLCAVRPTSGGSYIPTFGRGTS LIVHPY (SEQ ID NO: 204) TCRbeta extracellular sequence MGVTQTPKFQVLKTGQSMTLQCAQD MNHEYMSWYRQDPGMGLRLIHYSVG AGITDQGEVPNGYNVSRSTTEDFPLRL LSAAPSQTSVYFCASSYVGNTGELFFG EGSRLTVL (SEQ ID NO: 205) | T cells | 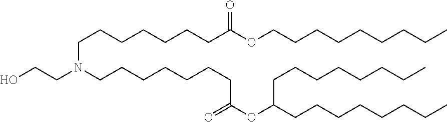 [SMILES: CCCCCCCCCOC(=O)CCCCCCCN(CCO)CCCCCCCC(=O)OC(CCCCCCCC)CCCCCCCC] (50 mol %) DSPC (10 mol %) Beta-sitosterol (28.5% mol %) Cholesterol (10 mol %) PEG DMG (1.5 mol %) |
| EPO | APPRLICDSRVLERYLLEAKEAENITTG | Kidney | |
| | CAEHCSLNENITVPDTKVNFYAWKRM | or | |
| | EVGQQAVEVWOGLALLSEAVLRGQA | bone | |
| | LLVNSSQPWEPLQLHVDKAVSGLRSLT | marrow | |
| | TLLRALGAQKEAISPPDAASAAPLRTIT | | |
| | ADTFRKLFRVYSNFLRGKLKLYTGEA | | |
| | CRTGDR (SEQ ID NO: 206) | | |
| PAH | MSTAVLENPGLGRKLSDFGQETSYIED NCNONGAISLIFSLKEEVGALAKVLRL FEENDVNLTHIESRPSRLKKDEYEFFTH LDKRSLPALTNIIKILRHDIGATVHELS RDKKKDTVPWFPRTIQELDRFANQILS YGAELDADHPGFKDPVYRARRKQFAD IAYNYRHGQPIPRVEYMEEEKKTWGT VFKTLKSLYKTHACYEYNHIFPLLEKY CGFHEDNIPQLEDVSQFLQTCTGFRLR PVAGLLSSRDFLGGLAFRVFHCTQYIR HGSKPMYTPEPDICHELLGHVPLFSDR SFAQFSQEIGLASLGAPDEYIEKLATIY WFTVEFGLCKQGDSIKAYGAGLLSSFG ELQYCLSEKPKLLPLELEKTAIQNYTV TEFQPLYYVAESFNDAKEKVRNFAATI PRPFSVRYDPYTQRIEVLDNTQQLKILA DSINSEIGILCSALQKIK (SEQ ID NO: 207) | Hepatic cells | 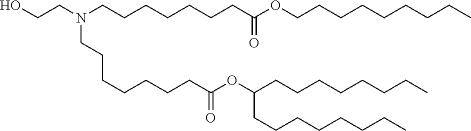 [SMILES: CCCCCCCCCOC(=O)CCCCCCCN(CCO)CCCCCCCC(=O)OC(CCCCCCCC)CCCCCCCC] (50 mol %) DSPC (10 mol %) Cholesterol (38.5% mol %) PEG-DMG (1.5%) OR MC3 (50 mol %) DSPC (10 mol %) Cholesterol (38.5% mol %) PEG-DMG (1.5%) |
| CPS1 | LSVKAQTAHIVLEDGTKMKGYSFGHP SSVAGEVVFNTGLGGYPEAITDPAYKG QILTMANPIIGNGGAPDTTALDELGLS KYLESNGIKVSGLLVLDYSKDYNHWL ATKSLGQWLQEEKVPAIYGVDTRMLT KIIRDKGTMLGKIEFEGQPVDFVDPNK QNLIAEVSTKDVKVYGKGNPTKVVAV DCGIKNNVIRLLVKRGAEVHLVPWNH DFTKMEYDGILIAGGPGNPALAEPLIQ NVRKILESDRKEPLFGISTGNLITGLAA GAKTYKMSMANRGQNQPVLNITNKQ AFITAQNHGYALDNTLPAGWKPLFVN VNDQTNEGIMHESKPFFAVQFHPEVTP GPIDTEYLFDSFFSLIKKGKATTITSVLP KPALVASRVEVSKVLILGSGGLSIGQA GEFDYSGSQAVKAMKEENVKTVLMN PNIASVQTNEVGLKQADTVYFLPITPQF VTEVIKAEQPDGLILGMGGQTALNCG VELFKRGVLKEYGVKVLGTSVESIMA TEDROLFSDKLNEINEKIAPSFAVESIE DALKAADTIGYPVMIRSAYALGGLGS GICPNRETLMDLSTKAFAMTNQILVEK SVTGWKEIEYEVVRDADDNCVTVCN MENVDAMGVHTGDSVVVAPAQTLSN AEFQMLRRTSINVVRHLGIVGECNIQF ALHPTSMEYCIIEVNARLSRSSALASKA TGYPLAFIAAKIALGIPLPEIKNVVSGK TSACFEPSLDYMVTKIPRWDLDRFHGT SSRIGSSMKSVGEVMAIGRTFEESFQK ALRMCHPSIEGFTPRLPMNKEWPSNLD LRKELSEPSSTRIYAIAKAIDDNMSLDE IEKLTYIDKWFLYKMRDILNMEKTLKG LNSESMTEETLKRAKEIGFSDKQISKCL GLTEAQTRELRLKKNIHPWVKQIDTLA AEYPSVTNYLYVTYNGQEHDVNFDDH GMMVLGCGPYHIGSSVEFDWCAVSSI RTLRQLGKKTVVVNCNPETVSTDFDE CDKLYFEELSLERILDIYHQEACGGCIIS VGGQIPNNLAVPLYKNGVKIMGTSPLQ IDRAEDRSIFSAVLDELKVAQAPWKAV NTLNEALEFAKSVDYPCLLRPSYVLSG SAMNVVFSEDEMKKFLEEATRVSQEH PVVLTKFVEGAREVEMDAVGKDGRVI SHAISEHVEDAGVHSGDATLMLPTQTI SQGAIEKVKDATRKIAKAFAISGPFNV QFLVKGNDVLVIECNLRASRSFPFVSK TLGVDFIDVATKVMIGENVDEKHLPTL DHPIIPADYVAIKAPMFSWPRLRDADPI LRCEMASTGEVACFGEGIHTAFLKAM LSTGFKIPQKGILIGIQQSFRPRFLGVAE QLHNEGFKLFATEATSDWLNANNVPA TPVAWPSQEGQNPSLSSIRKLIRDGSID LVINLPNNNTKFVHDNYVIRRTAVDSG IPLLTNFQVTKLFAEAVQKSRKVDSKS LFHYRQYSAGKAA (SEQ ID NO: 208) | Hepatic cells | 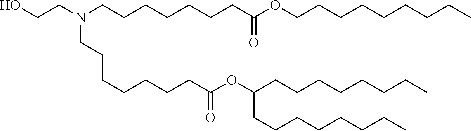 [SMILES: CCCCCCCCCOC(=O)CCCCCCCN(CCO)CCCCCCCC(=O)OC(CCCCCCCC)CCCCCCCC] (50 mol %) DSPC (10 mol %) Cholesterol (38.5% mol %) PEG-DMG (1.5%) OR MC3 (50 mol %) DSPC (10 mol %) Cholesterol (38.5% mol %) PEG-DMG (1.5%) |
| Cas9 | MKRNYILGLDIGITSVGYGIIDYETRDV IDAGVRLFKEANVENNEGRRSKRGAR RLKRRRRHRIQRVKKLLFDYNLLTDHS ELSGINPYEARVKGLSQKLSEEEFSAAL LHLAKRRGVHNVNEVEEDTGNELSTK EQISRNSKALEEKYVAELQLERLKKDG EVRGSINRFKTSDYVKEAKQLLKVQK AYHQLDQSFIDTYIDLLETRRTYYEGP GEGSPFGWKDIKEWYEMLMGHCTYFP EELRSVKYAYNADLYNALNDLNNL VI TRDENEKLEYYEKFQIIENVFKQKKKP TLKQIAKEILVNEEDIKGYRVTSTGKPE FTNLKVYHDIKDITARKEIIENAELLDQ IAKILTIYQSSEDIQEELTNLNSELTQEEI EQISNLKGYTGTHNLSLKAINLILDEL WHTNDNQIAIFNRLKLVPKKVDLSQQ KEIPTTLVDDFILSPVVKRSFIQSIKVIN AIIKKYGLPNDIIIELAREKNSKDAQKM INEMQKRNRQTNERIEEIIRTTGKENAK YLIEKIKLHDMQEGKCLYSLEAIPLEDL LNNPFNYEVDHIIPRSVSFDNSFNNKVL VKQEENSKKGNRTPFQYLSSSDSKISY ETFKKHILNLAKGKGRISKTKKEYLLE ERDINRFSVQKDFINRNLVDTRYATRG LMNLLRSYFRVNNLDVKVKSINGGFTS FLRRKWKFKKERNKGYKHHAEDALII ANADFIFKEWKKLDKAKKVMENQMF EEKQAESMPEIETEQEYKEIFITPHQIKH IKDFKDYKYSHRVDKKPNRELINDTLY STRKDDKGNTLIVNNLNGLYDKDNDK LKKLINKSPEKLLMYHHDPQTYQKLK LIMEQYGDEKNPLYKYYEETGNYLTK YSKKDNGPVIKKIKYYGNKLNAHLDIT DDYPNSRNKVVKLSLKPYRFDVYLDN GVYKFVTVKNLDVIKKENYYEVNSKC YEEAKKLKKISNQAEFIASFYNNDLIKI NGELYRVIGVNNDLLNRIEVNMIDITY REYLENMNDKRPPRIIKTIASKTQSIKK YSTDILGNLYEVKSKKHPQIIKKG (SEQ ID NO: 209) | Immune cells | 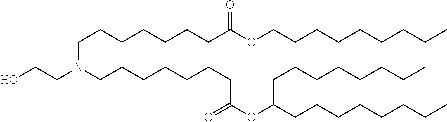 [SMILES: CCCCCCCCCOC(=O)CCCCCCCN(CCO)CCCCCCCC(=O)OC(CCCCCCCC)CCCCCCCC] (50 mol %) DSPC (10 mol %) Beta-sitosterol (28.5% mol %) Cholesterol (10 mol %) PEG DMG (1.5 mol %) |
| ADAMT S13 | AAGGILHLELLVAVGPDVFQAHQEDT ERYVLTNLNIGAELLRDPSLGAQFRVH LVKMVILTEPEGAPNITANLTSSLLSVC GWSQTINPEDDTDPGHADLVLYITRFD LELPDGNRQVRGVTQLGGACSPTWSC LITEDTGFDLGVTIAHEIGHSFGLEHDG APGSGCGPSGHVMASDGAAPRAGLA WSPCSRROLLSLLSAGRARCVWDPPRP QPGSAGHPPDAQPGLYYSANEQCRVA FGPKAVACTFAREHLDMCQALSCHTD PLDQSSCSRLLVPLLDGTECGVEKWCS KGRCRSLVELTPIAAVHGRWSSWGPR SPCSRSCGGGVVTRRRQCNNPRPAFGG RACVGADLQAEMCNTQACEKTQLEF MSQQCARTDGQPLRSSPGGASFYHWG AAVPHSQGDALCRHMCRAIGESFIMK RGDSFLDGTRCMPSGPREDGTLSLCVS GSCRTFGCDGRMDSQQVWDRCQVCG GDNSTCSPRKGSFTAGRAREYVTFLTV TPNLTSVYIANHRPLFTHLAVRIGGRY VVAGKMSISPNTTYPSLLEDGRVEYRV ALTEDRLPRLEEIRIWGPLQEDADIQVY RRYGEEYGNLTRPDITFTYFQPKPRQA WVWAAVRGPCSVSCGAGLRWVNYSC LDQARKELVETVQCQGSQQPPAWPEA CVLEPCPPYWAVGDFGPCSASCGGGL RERPVRCVEAQGSLLKTLPPARCRAGA QQPAVALETCNPQPCPARWEVSEPSSC TSAGGAGLALENETCVPGADGLEAPV TEGPGSVDEKLPAPEPCVGMSCPPGW GHLDATSAGEKAPSPWGSIRTGAQAA HVWTPAAGSCSVSCGRGLMELRFLCM DSALRVPVQEELCGLASKPGSRREVCQ AVPCPARWQYKLAACSVSCGRGVVR RILYCARAHGEDDGEEILLDTQCQGLP RPEPQEACSLEPCPPRWKVMSLGPCSA SCGLGTARRSVACVOLDQGQDVEVDE AACAALVRPEASVPCLIADCTYRWHV GTWMECSVSCGDGIQRRRDTCLGPQA QAPVPADFCQHLPKPVTVRGCWAGPC VGQGTPSLVPHEEAAAPGRTTATPAG ASLEWSQARGLLFSPAPQPRRLLPGPQ ENSVOSSACGRQHLEPTGTIDMRGPGQ ADCAVAIGRPLGEVVTLRVLESSLNCS AGDMLLLWGRLTWRKMCRKLLDMTF SSKTNTLVVRQRCGRPGGGVLLRYGS QLAPETFYRECDMQLFGPWGEIVSPSL SPATSNAGGCRLFINVAPHARIAIHALA TNMGAGTEGANASYILIRDTHSLRTTA FHGQQVLYWESESSQAEMEFSEGFLK AQASLRGQYWTLQSWVPEMQDPQSW KGKEGT (SEQ ID NO: 210) | Hepatic cells | 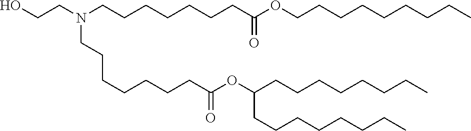 [SMILES: CCCCCCCCCOC(=O)CCCCCCCN(CCO)CCCCCCCC(=O)OC(CCCCCCCC)CCCCCCCC] (50 mol %) DSPC (10 mol %) Cholesterol (38.5% mol %) PEG-DMG (1.5%) OR MC3 (50 mol %) DSPC (10 mol %) Cholesterol (38.5% mol %) PEG-DMG (1.5%) |
| FOXP3 | MPNPRPGKPSAPSLALGPSPGASPSWR AAPKASDLLGARGPGGTFQGRDLRGG AHASSSSLNPMPPSQLQLPTLPLVMVA PSGARLGPLPHLQALLQDRPHFMHQLS TVDAHARTPVLQVHPLESPAMISLTPP TTATGVFSLKARPGLPPGINVASLEWV SREPALLCTFPNPSAPRKDSTLSAVPQS SYPLLANGVCKWPGCEKVFEEPEDFL KHCQADHLLDEKGRAQCLLQREMVQ SLEQQLVLEKEKLSAMQAHLAGKMAL TKASSVASSDKGSCCIVAAGSQGPVVP AWSGPREAPDSLFAVRRHLWGSHGNS TFPEFLHNMDYFKFHNMRPPFTYATLI RWAILEAPEKQRTLNEIYHWFTRMFAF FRNHPATWKNAIRHNLSLHKCFVRVE SEKGAVWTVDELEFRKKRSQRPSRCS NPTPGP (SEQ ID NO: 211) | Immune cells | 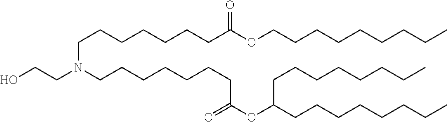 [SMILES: CCCCCCCCCOC(=O)CCCCCCCN(CCO)CCCCCCCC(=O)OC(CCCCCCCC)CCCCCCCC] (50 mol %) DSPC (10 mol %) Beta-sitosterol (28.5% mol %) Cholesterol (10 mol %) PEG DMG (1.5 mol %) |
| IL-10 | SPGQGTQSENSCTHFPGNLPNMLRDLR DAFSRVKTFFQMKDQLDNLLLKESLLE DFKGYLGCQALSEMIQFYLEEVMPQA ENQDPDIKAHVNSLGENLKTLRLRLRR CHRFLPCENKSKAVEQVKNAFNKLQE KGIYKAMSEFDIFINYIEAYMTMKIRN (SEQ ID NO: 212) | Immune cells | 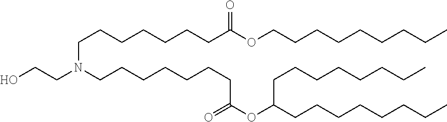 [SMILES: CCCCCCCCCOC(=O)CCCCCCCN(CCO)CCCCCCCC(=O)OC(CCCCCCCC)CCCCCCCC] (50 mol %) DSPC (10 mol %) Beta-sitosterol (28.5% mol %) Cholesterol (10 mol %) PEG DMG (1.5 mol %) |
| IL-2 | APTSSSTKKTQLQLEHLLLDLQMILNGI NNYKNPKLTRMLTFKFYMPKKATELK HLQCLEEELKPLEEVLNLAQSKNFHLR PRDLISNINVIVLELKGSETTFMCEYAD ETATIVEFLNRWITFCQSIISTLT (SEQ ID NO: 213) | Immune cells | 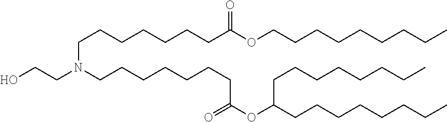 [SMILES: CCCCCCCCCOC(=O)CCCCCCCN(CCO)CCCCCCCC(=O)OC(CCCCCCCC)CCCCCCCC] (50 mol %) DSPC (10 mol %) Beta-sitosterol (28.5% mol %) Cholesterol (10 mol %) PEG DMG (1.5 mol %) |

In some embodiments, the expression sequence encodes a therapeutic protein. In some embodiments, the expression sequence encodes a cytokine, e.g., IL-12p70, IL-15, IL-2, IL-18, IL-21, IFN-α, IFN-β, IL-10, TGF-beta, IL-4, or IL-35, or a functional fragment thereof. In some embodiments, the expression sequence encodes an immune checkpoint inhibitor. In some embodiments, the expression sequence encodes an agonist (e.g., a TNFR family member such as CD137L, OX40L, ICOSL, LIGHT, or CD70). In some embodiments, the expression sequence encodes a chimeric antigen receptor. In some embodiments, the expression sequence encodes an inhibitory receptor agonist (e.g., PDL1, PDL2, Galectin-9, VISTA, B7H4, or MHCII) or inhibitory receptor (e.g., PD1, CTLA4, TIGIT, LAG3, or TIM3). In some embodiments, the expression sequence encodes an inhibitory receptor antagonist. In some embodiments, the expression sequence encodes one or more TCR chains (alpha and beta chains or gamma and delta chains). In some embodiments, the expression sequence encodes a secreted T cell or immune cell engager (e.g., a bispecific antibody such as BiTE, targeting, e.g., CD3, CD137, or CD28 and a tumor-expressed protein e.g., CD19, CD20, or BCMA etc.). In some embodiments, the expression sequence encodes a transcription factor (e.g., FOXP3, HELIOS, TOX1, or TOX2). In some embodiments, the expression sequence encodes an immunosuppressive enzyme (e.g., IDO or CD39/CD73). In some embodiments, the expression sequence encodes a GvHD (e.g., anti-HLA-A2 CAR-Tregs).

In some embodiments, circular RNA construct comprises an IRES and an expression sequence encoding a CAR. In certain embodiments, the circular RNA constructs and related pharmaceutical compositions herein comprise a CAR coding region that encodes a chimeric antigen receptor (CAR) complex protein. In certain embodiments, the expression sequence encodes a CAR targeting a cancer antigen. In certain embodiments, the CAR construct comprises, for example, an anti-CD19, anti-HER2, or anti-BCMA binder.

Chimeric antigen receptors (CARs or CAR-Ts) are genetically-engineered receptors. These engineered receptors may be inserted into and expressed by immune cells, including T cells via circular RNA as described herein. With a CAR a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell.

Accordingly, in some embodiments, the CAR encoded by the polynucleotide comprises (i) an antigen-binding molecule that specifically binds to a target antigen, (ii) a hinge domain, a transmembrane domain, and an intracellular domain, and (iii) an activating domain. In some embodiments, an orientation of the CARs in accordance with the disclosure comprises an antigen binding domain (such as an scFv) in tandem with a costimulatory domain and an activating domain. The costimulatory domain may comprise one or more of an extracellular portion, a transmembrane portion, and an intracellular portion. In other embodiments, multiple costimulatory domains may be utilized in tandem. In some embodiments, the CAR comprises a CAR protein spacer. The CAR protein spacer may be between any aforementioned domains. In some embodiments, the CAR is directed to a tumor-expressed protein, including but not limited to CD19, BCMA, and HER2.

### 1. Codon Optimization

In some embodiments where the circular RNA construct comprises at least one expression sequence encoding a binding molecule, the expression sequence may be codon optimized. In some embodiments, the circular RNA construct is optimized to lack at least one microRNA binding site present in an equivalent pre-optimized polynucleotide. In some embodiments, the circular RNA construct is optimized to lack at least one microRNA binding site capable of binding to a microRNA present in a cell within which the circular RNA construct is expressed. In some embodiments, the circular RNA construct is optimized to lack at least one endonuclease susceptible site present in an equivalent pre-optimized polynucleotide. In some embodiments, the circular RNA construct is optimized to lack at least one endonuclease susceptible site capable of being cleaved by an endonuclease present in a cell within which the endonuclease is expressed. In some embodiments, the circular RNA construct is optimized to lack at least one RNA editing susceptible site present in an equivalent pre-optimized polynucleotide.

A codon optimized sequence may be one in which codons in a polynucleotide encoding a polypeptide have been substituted in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid. In some embodiments, a codon optimized polynucleotide may minimize ribozyme collisions and/or limit structural interference between the expression sequence and the IRES.

Codon optimization can be performed by methods known in the art using known algorithms. A factor that can influence codon optimization includes differences in ribosomal dwell times among sequences. Optimization based on ribosomal dwell time prioritizes low dwell-time codons, namely codons that the ribosome does not linger on for very long (associated with translation speed). These codons tend to have a lower GC with a target GC % of around, for example, 48-54%. RNA stability also influences codon optimization. Optimization based on modified stability prioritizes codons associated with RNA stability, which also tend to have a higher GC, with a target GC % of around, for example, 57-62%. Higher-GC codons tend to improve stability by forming small structures in the RNA that constrain the reactive 2′ hydroxyl and for small segments of double-stranded RNA that may be resistant to endonuclease activity. These structures may also slow the ribosome and prevent it from bumping into other ribosomes. Another method of codon optimization utilizes an algorithm with specific codon usage reverse engineered from a known sequence, which has a target GC % of around 57-62%.

In embodiments using these algorithms, each algorithm will generate a random sequence that adheres to the codon usage frequencies that are associated with the algorithm. For most amino acids, multiple codons can be used (however, certain algorithms can select a single pre-defined codon for each amino acid). For the algorithms employing multiple codons, a single amino acid sequence input can have many different nucleotide sequence outputs, since the choice of codon at each position is random, and weighted. The algorithms generally exclude rare codons, which are defined slightly differently for each algorithm, but usually by codon usage in the corresponding genome. Optimization based on ribosomal dwell time and modified stability, for example use a biased codon matrix to generate a first pass sequence. The first pass bias can be, for example, set to 10. This means that, for example, for an amino acid with two potential codons at 0.6 and 0.4 usage (60%/40%), a bias of 10 makes 0.6 become: (0.6∧10)/((0.6∧10)−(0.4∧10))=˜0.98, and (0.4∧10)/((0.6∧10)−(0.4∧10))=˜0.02, resulting in a 98% chance of selecting the originally 0.6 codon, and 2% chance of selecting the originally 0.4 codon.

The primary sequence will then go through a “polishing process” to identify “problem sequences” such as: (a) self-complement regions that comprise greater than 11 contiguous nucleotides; (b) repeat regions of greater than 11 contiguous nucleotides; and (c) unwanted sequences, which include but are not limited to XbaI sites (TCTAGA), which are used to linearize plasmid and must not be present in the circular RNA region; 5+homopolymers (e.g., AAAAA), which can result in frameshift mutations in the RNA; and >75% or <33% GC % content over an 18nt window.

Once these problem sequences are identified, the algorithm will attempt to remove them by re-selecting the codon at a random position within the problematic sequence in question. The algorithm will loop, e.g., up to 25 times to remove each problem sequence, and then repeat this process, e.g., 25 times to remove each problem sequence. If the algorithm is unable to remove problem sequences, it will report the presence of problem sequences alongside the final sequence- and then repeat this process any number of times to generate unique sequences based on the same amino acid sequence.

### 2. Exemplary Antigen Binding Domains

In some embodiments, the circular RNA constructs comprise an IRES and at least one expression sequence encoding a binding molecule. In certain embodiments, the expression sequence encodes a therapeutic protein, for example a chimeric antigen receptor (CAR).

CARs may be engineered to bind to an antigen (such as a cell-surface antigen) by incorporating an antigen binding molecule that interacts with that targeted antigen, for example a cancer antigen. In some embodiments, the antigen binding molecule is an antibody fragment thereof, e.g., one or more single chain antibody fragment (scFv). An scFv is a single chain antibody fragment having the variable regions of the heavy and light chains of an antibody linked together. See U.S. Pat. Nos. 7,741,465, and 6,319,494 as well as Eshhar et al., Cancer Immunol Immunotherapy (1997) 45:131-136. An scFv retains the parent antibody's ability to specifically interact with target antigen. scFvs are useful in chimeric antigen receptors because they may be engineered to be expressed as part of a single chain along with the other CAR components. Id. See also Krause et al., J. Exp. Med., Volume 188, No. 4, 1998 (619-626); Finney et al., Journal of Immunology, 1998, 161:2791-2797. It will be appreciated that the antigen binding molecule is typically contained within the extracellular portion of the CAR such that it is capable of recognizing and binding to the antigen of interest. Bispecific and multispecific CARs are contemplated, with specificity to more than one target of interest.

In some embodiments, the antigen binding molecule comprises a single chain, wherein the heavy chain variable region and the light chain variable region are connected by a linker. In some embodiments, the VH is located at the N terminus of the linker and the VL is located at the C terminus of the linker. In other embodiments, the VL is located at the N terminus of the linker and the VH is located at the C terminus of the linker. In some embodiments, the linker comprises at least about 5, at least about 8, at least about 10, at least about 13, at least about 15, at least about 18, at least about 20, at least about 25, at least about 30, at least about 35, at least about 40, at least about 45, at least about 50, at least about 60, at least about 70, at least about 80, at least about 90, or at least about 100 amino acids.

In some embodiments, the antigen binding molecule comprises a nanobody. In some embodiments, the antigen binding molecule comprises a DARPin. In some embodiments, the antigen binding molecule comprises an anticalin or other synthetic protein capable of specific binding to target protein.

In some embodiments, the CAR comprises an antigen binding domain specific for an antigen selected from CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GaINAca-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis (Y) antigen, CD24, Platelet-derived growth factor receptor beta (PDGFR-beta), Stage-specific embryonic antigen-4 (SSEA-4), CD20, Folate receptor alpha, Human Epidermal Growth Factor Receptor 2 (HER2), HER3, Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-1 receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1), Cancer/testis antigen 2 (LAGE-1a), MAGE family members (including MAGE-A1, MAGE-A3 and MAGE-A4), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LA1R1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), MUC16, 5T4, 8H9, αvβ0 integrin, αvβ6 integrin, alphafetoprotein (AFP), B7-H6, ca-125, CA9, CD44, CD44v7/8, CD52, E-cadherin, EMA (epithelial membrane antigen), epithelial glycoprotein-2 (EGP-2), epithelial glycoprotein-40 (EGP-40), ErbB4, epithelial tumor antigen (ETA), folate binding protein (FBP), kinase insert domain receptor (KDR), k-light chain, L1 cell adhesion molecule, MUC18, NKG2D, oncofetal antigen (h5T4), tumor/testis-antigen 1B, GAGE, GAGE-1, BAGE, SCP-1, CTZ9, SAGE, CAGE, CT10, MART-1, immunoglobulin lambda-like polypeptide 1 (IGLL1), Hepatitis B Surface Antigen Binding Protein (HBsAg), viral capsid antigen (VCA), early antigen (EA), EBV nuclear antigen (EBNA), HHV-6 p41 early antigen, HHV-6B U94 latent antigen, HHV-6B p98 late antigen, cytomegalovirus (CMV) antigen, large T antigen, small T antigen, adenovirus antigen, respiratory syncytial virus (RSV) antigen, hacmagglutinin (HA), neuraminidase (NA), parainfluenza type 1 antigen, parainfluenza type 2 antigen, parainfluenza type 3 antigen, parainfluenza type 4 antigen, Human Metapneumovirus (HMPV) antigen, hepatitis C virus (HCV) core antigen, HIV p24 antigen, human T-cell lympotrophic virus (HTLV-1) antigen, Merkel cell polyoma virus small T antigen, Merkel cell polyoma virus large T antigen, Kaposi sarcoma-associated herpesvirus (KSHV) lytic nuclear antigen and KSHV latent nuclear antigen.

As a non-limiting example, in some embodiments, the circular RNA construct comprises an IRES and at least one expression sequence encoding a CAR targeting a cancer antigen. As a non-limiting example, in some embodiments, the circular RNA construct comprises an IRES and a CAR comprising an antigen binding domain specific for CD19. In some embodiments, the circular RNA construct comprises an IRES and a CAR comprising an antigen binding domain specific for BCMA. In some embodiments, the circular RNA construct comprises an IRES and a CAR comprising an antigen binding domain specific for HER2. In some embodiments, the expression sequence is codon optimized.

As a non-limiting example, in some embodiments, the circular RNA construct comprises a CAR comprising an antigen binding domain specific for CD19 (B-lymphocyte antigen CD19). CD19 is a biomarker for normal and neoplastic B cells, as well as follicular dendritic cells. Diffuse large B cell lymphoma (DLBCL) is the most common lymphoma, accounting for about 25% to 30% of all the non-Hodgkin lymphomas, followed by FL. As CD19 is expressed in over 95% of B-cell malignancies, it is an attractive target for immunotherapeutic approaches. One known example of a CAR T cell therapy targeting CD19 is Yescarta® (Kite Pharma Inc., axicabtagene ciloleucel), an anti-CD19 28-ζ(28-zeta) CAR. Another known example of a CAR T cell therapy targeting CD19 is Kymriah® (Novartis Pharmaceutical Corp., tisagenlecleucel), an anti-CD19 BB-((BB-zeta) CAR. Accordingly, in some embodiments, the expression sequence of the circular RNA construct encodes a CAR, where the codon is directed to an anti-CD19 domain known in the art. In some embodiments, the CAR construct comprises an anti-CD19 binder. In some embodiments, the expression sequence is codon optimized.

As a further non-limiting example, in some embodiments, the circular RNA construct comprises a CAR comprising an antigen binding domain specific for B-cell maturation antigen (BCMA). BCMA (also referred to as TNFRSF17 or CD269), is a member of the tumor necrosis factor receptor (TNFR) superfamily and is expressed by normal and malignant plasma cells and a small subset of B cells. BCMA a known biomarker for certain cancers, including multiple myeloma, and several BCMA-targeted CAR T therapies have been studied, where the constructs varied in their costimulatory domains, hinge regions, transmembrane domains, species used to generate the anti-BCMA scFVs, and the use of different modifications to address safety of the CAR-T therapy. See generally Shah et al., “B-cell maturation antigen (BCMA) in multiple myeloma: rationale for targeting and current therapeutic approaches,” Leukemia 34, 985-1005 (2020). Accordingly, in some embodiments, the expression sequence of the circular RNA construct encodes a CAR, where the codon is directed to an anti-BCMA domain known in the art. In some embodiments, the CAR construct comprises an anti-BCMA binder. In some embodiments, the expression sequence is codon optimized.

As a further non-limiting example, in some embodiments, the circular RNA construct comprises a CAR comprising an antigen binding domain specific for Human Epidermal Growth Factor Receptor 2 (HER2). For example, the CAR can be directed to HER2-BB-((BB-zeta) and/or HER2-286 (28-zeta). Accordingly, in some embodiments, the CAR construct comprises an anti-HER2 binder. In some embodiments, the expression sequence is codon optimized.

In some embodiments, the circular RNA constructs and related pharmaceutical compositions comprise the expression sequences described in Tables 2A-2C below. In some embodiments, the circular RNA constructs and related pharmaceutical compositions disclosed herein comprise an expression sequence that is at least 80%, 85%, 90%, 91%, 92%, 93%, 94%, 95%, 96%, 97%, 98%, or 99% identical to a sequence in Tables 2A-2C, wherein the codon sequence produces a protein having the desired sequence.

The exemplary anti-CD19 binder sequences in Table 2A are codon-optimized and correspond to an anti-CD19 28-¿ (28 zeta) CAR. The amino acid sequence corresponding to the nucleotide sequences in Table 2A is set forth in SEQ ID NO: 29:

(SEQ ID NO: 29)
MALPVTALLLPLALLLHAARPDIQMTQTTSSLSASLGDRVTISCRASQD
ISKYLNWYQQKPDGTVKLLIYHTSRLHSGVPSRFSGSGSGTDYSLTISN
LEQEDIATYFCQQGNTLPYTFGGGTKLEITGGGGSGGGGSGGGGSEVKL
QESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYG
GSYAMDYWGQGTSVTVSSIEVMYPPPYLDNEKSNGTIIHVKGKHLCPSP
LFPGPSKPFWVLVVVGGVLACYSLLVTVAFIIFWVRSKRSRLLHSDYMN
MTPRRPGPTRKHYQPYAPPRDFAAYRSRVKFSRSADAPAYQQGQNQLYN
ELNLGRREEYDVLDKRRGRDPEMGGKPRRKNPQEGLYNELQKDKMAEAY
SEIGMKGERRRGKGHDGLYQGLSTATKDTYDALHMQALPPR

In some embodiments, the circular RNA constructs and related pharmaceutical compositions disclosed herein comprise a CAR sequence encoding a polypeptide that comprises at least 80%, 85%, 90%, 91%, 92%, 93%, 94%, 95%, 96%, 97%, 98%, or 99% identity to SEQ ID NO: 29 or binding fragment thereof.

In some embodiments, the circular RNA constructs and related pharmaceutical compositions disclosed herein comprise an IRES sequence having at least 85%, 86%, 87%, 88%, 89%, 90%, 91%, 92%, 93%, 94%, 95%, 96%, 97%, 98%, 99%, or 100% sequence identity to an IRES from Table IA, an IRES from a construct of any one of SEQ ID NOs: 50-61 or any of Constructs A-P of Table 1B, and a CAR sequence encoding a polypeptide comprising at least 85%, 86%, 87%, 88%, 89%, 90%, 91%, 92%, 93%, 94%, 95%, 96%, 97%, 98%, 99%, or 100% sequence identity to SEQ ID NO: 29 or binding fragment thereof. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

TABLE 2A
Codon Optimized Sequences (anti-CD19 28-ζ)

| Codon | SEQ ID | |
|---|---|---|
| NO: | NO: | NT SEQUENCE |
| 2A-19 | 19 | ATGGCACTGCCCGTCACCGCACTCCTGCTCCCACTGGCACTGCTGCTCCATGCAG |
| | | CTCGCCCCGATATCCAGATGACCCAGACCACCTCTAGCCTCAGCGCCTCTCTGGG |
| | | TGACCGCGTCACCATCTCTTGCCGGGCCAGCCAAGACATCTCTAAGTACCTGAA |
| | | CTGGTACCAGCAGAAACCTGACGGAACCGTGAAGCTGCTGATCTACCACACCAG |
| | | TCGGCTGCATTCCGGGGTGCCTTCCAGGTTCAGCGGTTCCGGCTCTGGGACCGAT |
| | | TATAGTCTCACCATCTCCAACCTCGAGCAGGAGGACATCGCAACCTACTTCTGCC |
| | | AGCAGGGGAACACCCTGCCCTACACCTTCGGTGGCGGGACCAAGCTGGAGATCA |
| | | CTGGAGGTGGTGGCAGCGGAGGTGGAGGATCAGGTGGAGGCGGTAGCGAGGTG |
| | | AAGCTGCAGGAGTCCGGACCTGGTCTGGTGGCCCCAAGCCAGTCCCTCAGCGTC |
| | | ACCTGCACAGTGTCCGGGGTGTCCCTGCCTGACTACGGTGTCTCCTGGATCAGGC |
| | | AACCACCCCGGAAGGGTCTCGAGTGGCTGGGCGTCATCTGGGGCTCCGAGACCA |
| | | CCTACTACAACAGCGCTCTGAAGTCCCGGCTGACCATCATCAAAGACAACTCCA |
| | | AGAGCCAGGTGTTCTTGAAGATGAACTCCCTGCAAACCGATGACACCGCCATCT |
| | | ACTACTGCGCCAAGCACTACTACTATGGCGGTAGCTACGCCATGGATTATTGGG |
| | | GTCAGGGCACCAGTGTCACCGTCTCCTCCATCGAGGTGATGTACCCTCCACCCTA |
| | | TCTGGACAACGAGAAGTCCAACGGCACCATCATCCACGTGAAGGGCAAGCACCT |
| | | GTGCCCTAGCCCTCTGTTCCCAGGACCCTCCAAGCCCTTCTGGGTGCTGGTCGTG |
| | | GTGGGAGGAGTCCTGGCCTGCTATTCCCTCCTCGTCACCGTGGCATTTATCATCT |
| | | TCTGGGTCCGGAGCAAGCGGTCACGCCTGCTCCACTCCGACTACATGAACATGA |
| | | CTCCTCGCAGACCTGGACCCACCCGGAAGCACTACCAGCCTTATGCCCCACCCC |
| | | GCGACTTTGCCGCTTACCGCTCTCGGGTCAAGTTCTCTCGGTCAGCAGACGCCCC |
| | | TGCATACCAGCAGGGCCAGAACCAGCTGTATAACGAGCTGAACCTCGGCAGAC |
| | | GGGAGGAGTACGATGTGCTGGACAAGAGGAGAGGCAGAGACCCCGAGATGGGT |
| | | GGTAAGCCACGGCGCAAGAACCCACAGGAGGGCTTGTACAACGAACTGCAGAA |
| | | GGACAAGATGGCCGAGGCCTACAGCGAGATCGGCATGAAGGGAGAGAGGCGCA |
| | | GGGGCAAGGGTCACGACGGCCTGTACCAAGGGCTGTCCACCGCAACCAAGGAC |
| | | ACCTACGATGCCCTGCACATGCAGGCCCTCCCACCAAGG |
| 2A-20 | 20 | ATGGCACTCCCAGTCACCGCACTTCTGCTGCCTCTCGCCCTGCTGCTCCATGCAG |
| | | CCAGACCCGACATCCAGATGACCCAAACCACCAGCTCCCTGTCCGCTTCCCTGG |
| | | GTGACCGGGTGACTATCTCTTGCCGGGCCTCCCAAGACATCTCCAAGTACCTGA |
| | | ACTGGTATCAGCAAAAGCCTGACGGCACCGTCAAGCTCCTCATCTACCATACCT |
| | | CCAGACTGCACTCCGGGGTGCCTAGCAGGTTCAGCGGAAGTGGGAGCGGCACC |
| | | GACTACAGCCTCACCATCTCCAACCTGGAGCAGGAGGACATCGCCACCTACTTC |
| | | TGCCAGCAGGGGAACACACTGCCCTACACCTTCGGCGGTGGCACCAAGCTGGAG |
| | | ATCACAGGTGGCGGAGGTTCCGGAGGAGGAGGTAGTGGAGGTGGAGGCAGCGA |
| | | GGTGAAGCTCCAGGAATCCGGACCAGGTCTGGTGGCTCCCAGCCAGTCCCTCAG |
| | | CGTGACCTGCACCGTGAGCGGCGTGTCTCTTCCCGATTACGGAGTGTCCTGGATC |
| | | AGACAGCCACCCCGGAAGGGTCTGGAGTGGCTGGGAGTGATCTGGGGTTCCGA |
| | | GACCACATACTACAACTCAGCCCTCAAGAGCCGGCTCACCATCATCAAGGATAA |
| | | CTCCAAGTCCCAGGTCTTCCTGAAGATGAACTCTCTCCAGACCGACGACACCGC |
| | | CATCTACTACTGCGCCAAGCACTACTACTACGGCGGGTCCTACGCCATGGACTA |
| | | CTGGGGTCAGGGAACCTCCGTCACCGTCAGCTCTATCGAGGTGATGTACCCTCCT |
| | | CCCTACCTCGACAACGAGAAGAGCAACGGCACCATCATCCATGTGAAGGGGAA |
| | | GCATCTCTGCCCCTCACCCCTGTTCCCCGGACCATCCAAGCCATTCTGGGTGCTG |
| | | GTGGTTGTTGGTGGGGTCCTGGCTTGCTACTCACTCCTGGTCACCGTCGCCTTCA |
| | | TCATCTTCTGGGTGCGGTCAAAGAGGTCCCGGCTCTTGCACTCCGATTACATGAA |
| | | CATGACTCCAAGGAGGCCTGGTCCCACACGGAAGCACTACCAACCATATGCCCC |
| | | ACCACGCGACTTCGCTGCTTACCGGAGCCGGGTCAAGTTCAGTCGGAGTGCAGA |
| | | CGCCCCAGCCTACCAGCAGGGCCAGAACCAACTCTACAACGAGCTTAATCTGGG |
| | | TCGCCGGGAGGAGTATGACGTGCTCGATAAGAGAAGGGGCCGGGATCCTGAGA |
| | | TGGGCGGTAAGCCCAGACGGAAGAACCCTCAGGAGGGGTTGTATAATGAGCTC |
| | | CAGAAGGACAAGATGGCCGAGGCATACTCCGAGATCGGCATGAAAGGTGAGCG |
| | | GAGGAGAGGCAAGGGGCATGACGGCCTGTACCAGGGGCTCAGCACAGCCACCA |
| | | AGGATACCTATGACGCACTCCACATGCAGGCACTGCCTCCACGG |
| 2A-21 | 21 | ATGGCTCTGCCTGTGACAGCTCTGCTGCTGCCTCTGGCTCTGCTTCTGCATGCCG |
| | | CCAGACCTGACATCCAGATGACCCAGACAACCAGCAGCCTGTCTGCCAGCCTGG |
| | | GCGATAGAGTGACCATCAGCTGTAGAGCCAGCCAGGACATCAGCAAGTACCTG |
| | | AACTGGTATCAGCAGAAACCCGACGGCACCGTGAAGCTGCTGATCTACCACACC |
| | | AGCAGACTGCACAGCGGCGTGCCAAGCAGATTTTCTGGCAGCGGCTCTGGCACC |
| | | GACTACAGCCTGACAATCAGCAACCTGGAACAAGAGGATATCGCTACCTACTTC |
| | | TGCCAGCAAGGCAACACCCTGCCTTACACCTTTGGCGGAGGCACCAAGCTGGAA |
| | | ATCACAGGCGGCGGAGGAAGCGGAGGCGGAGGATCTGGTGGTGGTGGATCTGA |
| | | AGTGAAACTGCAAGAGTCTGGCCCTGGCCTGGTGGCCCCATCTCAATCTCTGAG |
| | | CGTGACCTGTACCGTCAGCGGAGTGTCCCTGCCTGATTATGGCGTGTCCTGGATC |
| | | CGGCAGCCTCCTAGAAAAGGCCTGGAATGGCTGGGCGTGATCTGGGGCAGCGA |
| | | GACAACCTACTACAACAGCGCCCTGAAGTCCCGGCTGACCATCATCAAGGACAA |
| | | CTCCAAGAGCCAGGTGTTCCTGAAGATGAACAGCCTGCAGACCGACGACACCGC |
| | | CATCTACTATTGCGCCAAGCACTACTACTACGGCGGCAGCTACGCCATGGATTA |
| | | TTGGGGCCAGGGCACCAGCGTGACCGTGTCTAGCATCGAAGTGATGTACCCTCC |
| | | ACCTTACCTGGACAACGAGAAGTCCAACGGCACCATCATCCACGTGAAGGGCAA |
| | | GCACCTGTGTCCTTCTCCACTGTTCCCCGGACCTAGCAAGCCTTTCTGGGTGCTC |
| | | GTTGTTGTTGGCGGCGTGCTGGCCTGTTACTCTCTGCTGGTTACCGTGGCCTTCA |
| | | TCATCTTTTGGGTCCGAAGCAAGCGGAGCCGGCTGCTGCACTCCGACTACATGA |
| | | ACATGACCCCTAGACGGCCCGGACCAACCAGAAAGCACTACCAGCCTTACGCTC |
| | | CTCCTAGAGACTTCGCCGCCTACCGGTCCAGAGTGAAGTTCAGCAGATCCGCCG |
| | | ATGCTCCCGCCTATCAGCAGGGCCAAAACCAGCTGTACAACGAGCTGAACCTGG |
| | | GGAGAAGAGAAGAGTACGACGTGCTGGACAAGCGGAGAGGCAGAGATCCTGAA |
| | | ATGGGCGGCAAGCCCAGACGGAAGAATCCTCAAGAGGGCCTGTATAATGAGCT |
| | | GCAGAAAGACAAGATGGCCGAGGCCTACAGCGAGATCGGAATGAAGGGCGAGC |
| | | GCAGAAGAGGCAAGGGACACGATGGACTGTACCAGGGCCTGAGCACCGCCACC |
| | | AAGGATACCTATGATGCCCTGCACATGCAGGCCCTGCCTCCAAGA |
| 2A-22 | 22 | ATGGCCCTTCCCGTCACCGCTCTCCTCCTGCCACTGGCCTTGCTGCTGCACGCTG |
| | | CACGGCCAGACATCCAGATGACCCAGACAACCAGCTCTCTGTCAGCCTCTCTCG |
| | | GCGATCGCGTCACAATCAGCTGCCGCGCTTCCCAAGACATCTCCAAGTACCTGA |
| | | ACTGGTACCAGCAAAAGCCCGACGGCACCGTGAAGCTGCTCATCTACCACACCT |
| | | CCAGACTGCATAGCGGGGTGCCCAGCAGATTCAGTGGCTCAGGCTCAGGCACCG |
| | | ACTACAGCCTGACCATCTCCAACCTGGAGCAGGAGGACATTGCCACATACTTCT |
| | | GCCAGCAGGGCAACACCCTGCCCTACACCTTCGGAGGCGGCACAAAGCTGGAG |
| | | ATCACCGGTGGAGGAGGGAGTGGAGGAGGAGGCAGTGGTGGCGGAGGTTCCGA |
| | | GGTGAAGCTCCAGGAATCAGGTCCAGGACTGGTCGCCCCTTCCCAGTCCCTGTC |
| | | CGTCACCTGCACCGTGAGTGGCGTCAGCCTCCCAGACTACGGTGTGTCTTGGATC |
| | | CGCCAACCTCCTCGCAAAGGCCTGGAATGGCTCGGCGTCATCTGGGGAAGCGAG |
| | | ACAACCTACTATAACTCCGCACTGAAGTCCCGCCTCACCATCATCAAGGATAAT |
| | | AGCAAGAGCCAGGTCTTCCTCAAGATGAACTCCCTGCAGACCGACGATACCGCC |
| | | ATCTACTACTGTGCCAAGCACTACTACTACGGAGGTTCTTACGCCATGGATTACT |
| | | GGGGACAGGGAACCTCTGTCACCGTCAGCTCCATCGAGGTCATGTATCCACCAC |
| | | CCTACCTGGACAACGAAAAGAGCAATGGCACCATCATCCACGTGAAGGGGAAG |
| | | CACCTCTGCCCCTCACCCCTGTTCCCTGGTCCCTCCAAGCCTTTCTGGGTCCTGGT |
| | | CGTCGTGGGAGGCGTGTTGGCCTGTTACTCCCTGCTCGTCACCGTCGCCTTCATC |
| | | ATCTTCTGGGTTAGGAGTAAGCGGTCCCGGCTTCTGCACTCTGACTACATGAACA |
| | | TGACACCCAGAAGACCTGGGCCAACCCGGAAGCACTACCAGCCCTACGCTCCAC |
| | | CCAGGGACTTTGCAGCCTACAGGTCCCGCGTCAAGTTCTCCCGGTCTGCTGACGC |
| | | ACCTGCCTACCAGCAGGGCCAAAACCAGCTCTACAACGAGTTGAACCTCGGCAG |
| | | ACGGGAGGAGTACGACGTCCTCGACAAAAGGGGGGTCGGGATCCTGAGATGG |
| | | GCGGTAAGCCAAGGCGGAAGAACCCACAGGAAGGCCTCTATAATGAGCTCCAG |
| | | AAGGATAAGATGGCTGAGGCCTACTCCGAGATCGGGATGAAGGGCGAAAGGAG |
| | | ACGGGGTAAGGGGCACGACGGCCTCTATCAGGGTCTGAGCACCGCCACCAAGG |
| | | ACACCTACGACGCCCTGCACATGCAGGCACTGCCACCTCGG |
| 2A-23 | 23 | ATGGCTCTGCCAGTGACCGCACTGCTGCTGCCCTTAGCCTTACTCCTTCACGCAG |
| | | CCAGGCCCGACATCCAGATGACCCAGACCACCAGCTCCCTTTCCGCAAGCCTCG |
| | | GCGACAGGGTCACCATCTCCTGTCGGGCCAGCCAGGACATCAGCAAGTACCTGA |
| | | ACTGGTACCAGCAGAAGCCCGACGGCACCGTGAAGCTGCTGATCTACCACACCT |
| | | CACGGCTGCACTCAGGCGTGCCCTCACGGTTTAGCGGATCAGGCAGCGGCACCG |
| | | ACTACAGCCTGACTATCAGCAACCTGGAGCAGGAGGACATCGCCACCTACTTCT |
| | | GCCAGCAGGGCAACACCCTGCCCTACACCTTCGGAGGCGGCACCAAGCTGGAG |
| | | ATCACCGGTGGCGGTGGTTCAGGTGGCGGAGGCTCAGGAGGAGGCGGCAGCGA |
| | | GGTGAAGCTGCAGGAGTCAGGTCCAGGACTGGTGGCACCCAGCCAGAGCCTGA |
| | | GCGTGACTTGCACCGTGTCAGGCGTGAGCCTGCCAGACTACGGCGTGAGCTGGA |
| | | TCCGGCAGCCTCCTCGGAAGGGCTTAGAGTGGCTGGGCGTGATCTGGGGCAGCG |
| | | AGACCACCTACTACAACTCAGCCCTGAAGAGCCGGCTGACCATCATCAAGGACA |
| | | ACAGCAAGAGCCAGGTGTTCCTGAAGATGAACAGCCTGCAGACCGACGACACC |
| | | GCCATCTACTACTGCGCCAAGCACTACTACTACGGCGGCAGCTACGCCATGGAC |
| | | TACTGGGGACAGGGTACCAGCGTGACCGTGAGCAGCATCGAGGTGATGTACCCT |
| | | CCTCCCTACCTGGACAACGAGAAGAGCAACGGCACCATCATCCACGTGAAGGGC |
| | | AAGCACCTGTGCCCTAGCCCTTTATTCCCCGGCCCCTCAAAACCCTTCTGGGTGC |
| | | TGGTCGTCGTCGGTGGCGTGCTGGCATGCTACAGCCTGCTGGTGACCGTGGCCTT |
| | | CATCATATTCTGGGTCCGGTCAAAGCGGAGCCGGTTACTGCACAGCGACTACAT |
| | | GAACATGACTCCACGGCGTCCAGGTCCCACTCGGAAGCACTACCAACCCTACGC |
| | | TCCTCCCCGTGACTTTGCTGCCTACCGTAGCCGGGTGAAGTTCTCCAGGAGCGCC |
| | | GATGCCCCAGCCTACCAGCAGGGCCAGAACCAGCTCTACAATGAGCTTAACCTT |
| | | GGCAGGCGGGAGGAGTACGACGTGCTGGACAAGAGGAGGGGCCGTGATCCCGA |
| | | GATGGGAGGCAAGCCCCGTAGGAAGAATCCCCAGGAGGGCCTTTACAACGAGC |
| | | TCCAGAAGGACAAGATGGCCGAGGCCTACAGCGAGATCGGCATGAAGGGAGAG |
| | | CGTAGGCGTGGAAAGGGCCACGACGGCCTGTACCAGGGCCTGAGCACTGCTACC |
| | | AAGGACACCTACGACGCCCTGCACATGCAGGCTCTTCCACCCCGG |

Table 2B sets forth nucleotide and amino acid sequences for additional exemplary anti-CD19 binder sequences that are not codon-optimized. The sequences are directed to an anti-CD19 28-ζ(28 zeta) CAR. In some embodiments, the circular RNA constructs and related pharmaceutical compositions disclosed herein comprise a CAR sequence encoding a polypeptide that comprises at least 80%, 85%, 90%, 91%, 92%, 93%, 94%, 95%, 96%, 97%, 98%, or 99% identity to any one of SEQ ID NO: 30-34 or binding fragments thereof.

In some embodiments, the circular RNA constructs and related pharmaceutical compositions disclosed herein comprise an IRES sequence having at least 85%, 86%, 87%, 88%, 89%, 90%, 91%, 92%, 93%, 94%, 95%, 96%, 97%, 98%, 99%, or 100% sequence identity to an IRES from Table 1A, an IRES from a construct of any one of SEQ ID NOs: 50-61 or any of Constructs A-P of Table 1B, and a CAR sequence encoding a polypeptide comprising at least 85%, 86%, 87%, 88%, 89%, 90%, 91%, 92%, 93%, 94%, 95%, 96%, 97%, 98%, 99%, or 100% sequence identity to any one of SEQ ID NO: 30-34 or binding fragments thereof. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

TABLE 2B
Additional Codon Amino Acid and Nucleotide Sequences (anti-CD19 28-ζ)

| SEQ | | SEQ | |
|---|---|---|---|
| ID | | ID | |
| NO | Codon nucleotide sequence | NO | Codon amino acid sequence |
| 24 | ATGCTCCTCCTGGTGACCAGCTTGCTCC | 30 | MLLLVTSLLLCELPHPAFLLIPDIQMTQT |
| | TGTGCGAACTGCCACACCCCGCCTTCCT | | TSSLSASLGDRVTISCRASQDISKYLNWY |
| | CCTCATCCCCGATATCCAGATGACCCAG | | QQKPDGTVKLLIYHTSRLHSGVPSRFSG |
| | ACCACCTCCTCCCTGAGCGCAAGCCTCG | | SGSGTDYSLTISNLEQEDIATYFCQQGNT |
| | GCGATCGGGTGACCATCTCATGCAGGG | | LPYTFGGGTKLEITGSTSGSGKPGSGEGS |
| | CCTCCCAGGACATCTCCAAGTATCTGAA | | TKGEVKLQESGPGLVAPSQSLSVTCTVS |
| | CTGGTATCAGCAGAAGCCTGACGGCAC | | GVSLPDYGVSWIRQPPRKGLEWLGVIW |
| | CGTCAAGCTGCTCATCTACCACACCTCA | | GSETTYYNSALKSRLTIIKDNSKSQVFLK |
| | CGGCTGCACTCAGGCGTCCCCTCAAGAT | | MNSLQTDDTAIYYCAKHYYYGGSYAM |
| | TCAGCGGTAGCGGATCCGGGACCGACT | | DYWGQGTSVTVSSAAAIEVMYPPPYLD |
| | ACTCCCTTACCATCAGCAACCTGGAGCA | | NEKSNGTIIHVKGKHLCPSPLFPGPSKPF |
| | GGAGGATATCGCCACATACTTCTGCCAG | | WVLVVVGGVLACYSLLVTVAFIIFWVR |
| | CAGGGTAACACCCTGCCCTATACCTTCG | | SKRSRLLHSDYMNMTPRRPGPTRKHYQ |
| | GCGGTGGGACCAAGCTGGAGATCACCG | | PYAPPRDFAAYRSRVKFSRSADAPAYQQ |
| | GTTCTACATCCGGATCCGGCAAGCCTGG | | GQNQLYNELNLGRREEYDVLDKRRGRD |
| | TAGTGGCGAGGGCTCCACCAAAGGGGA | | PEMGGKPRRKNPQEGLYNELQKDKMA |
| | GGTGAAGCTGCAGGAGTCCGGTCCAGG | | EAYSEIGMKGERRRGKGHDGLYQGLST |
| | TCTGGTGGCTCCAAGTCAGTCCCTGTCT | | ATKDTYDALHMQALPPR |
| | GTGACTTGCACCGTGTCAGGCGTGAGCC | | |
| | TGCCTGACTACGGGGTGAGCTGGATCCG | | |
| | GCAGCCACCTCGGAAGGGGTTGGAGTG | | |
| | GCTGGGAGTCATCTGGGGATCCGAGAC | | |
| | CACCTACTACAATTCCGCCCTCAAAAGC | | |
| | CGCCTCACCATCATCAAGGACAACTCCA | | |
| | AGTCCCAGGTCTTCCTGAAGATGAATTC | | |
| | CCTGCAGACCGACGACACCGCTATCTAT | | |
| | TACTGCGCCAAGCATTACTACTACGGCG | | |
| | GGTCCTACGCCATGGACTACTGGGGTCA | | |
| | AGGCACCTCCGTCACTGTTTCCTCCGCA | | |
| | GCAGCCATCGAGGTCATGTATCCTCCTC | | |
| | CCTACCTCGACAACGAGAAGTCCAACG | | |
| | GGACCATCATCCACGTGAAGGGCAAGC | | |
| | ACCTCTGCCCAAGCCCACTGTTCCCAGG | | |
| | GCCCTCCAAACCATTCTGGGTGCTCGTG | | |
| | GTGGTGGGTGGCGTGCTCGCTTGCTACT | | |
| | CCCTCCTGGTCACCGTCGCCTTCATCAT | | |
| | CTTTTGGGTCCGGAGTAAGCGCAGCCGC | | |
| | CTGCTCCATAGCGACTACATGAACATGA | | |
| | CCCCACGGAGACCTGGTCCCACCCGGA | | |
| | AACACTACCAGCCCTACGCACCACCCA | | |
| | GGGACTTCGCTGCCTATCGGTCCCGGGT | | |
| | TAAATTCTCTAGGTCCGCTGATGCCCCA | | |
| | GCCTACCAGCAGGGCCAGAACCAGCTG | | |
| | TACAATGAGCTGAACCTGGGTAGACGG | | |
| | GAGGAGTATGACGTCCTGGATAAGCGC | | |
| | AGAGGGAGAGACCCCGAGATGGGTGGA | | |
| | AAGCCCAGGCGGAAGAATCCCCAGGAG | | |
| | GGTCTCTATAACGAGCTCCAGAAGGAC | | |
| | AAGATGGCCGAGGCCTACAGCGAGATC | | |
| | GGGATGAAAGGGGAAAGAAGGCGGGG | | |
| | AAAGGGCCATGACGGACTGTACCAGGG | | |
| | TCTGTCCACCGCTACCAAGGACACCTAC | | |
| | GATGCACTGCACATGCAGGCACTGCCTC | | |
| | CTCGG | | |
| 25 | ATGCTGCTTCTCGTTACATCTCTGTTGCT | 31 | MLLLVTSLLLCELPHPAFLLIPDIQMTQT |
| | CTGCGAGCTGCCTCATCCAGCCTTCCTC | | TSSLSASLGDRVTISCRASQDISKYLNWY |
| | CTGATTCCCGATATCCAGATGACCCAGA | | QQKPDGTVKLLIYHTSRLHSGVPSRFSG |
| | CCACCTCTAGCCTCAGCGCCTCTCTGGG | | SGSGTDYSLTISNLEQEDIATYFCQQGNT |
| | TGACCGCGTCACCATCTCTTGCCGGGCC | | LPYTFGGGTKLEITGSTSGSGKPGSGEGS |
| | AGCCAAGACATCTCTAAGTACCTGAACT | | TKGEVKLQESGPGLVAPSQSLSVTCTVS |
| | GGTACCAGCAGAAACCTGACGGAACCG | | GVSLPDYGVSWIRQPPRKGLEWLGVIW |
| | TGAAGCTGCTGATCTACCACACCAGTCG | | GSETTYYNSALKSRLTIIKDNSKSQVFLK |
| | GCTGCATTCCGGGGTGCCTTCCAGGTTC | | MNSLQTDDTAIYYCAKHYYYGGSYAM |
| | AGCGGTTCCGGCTCTGGGACCGATTATA | | DYWGQGTSVTVSSAAAIEVMYPPPYLD |
| | GTCTCACCATCTCCAACCTCGAGCAGGA | | NEKSNGTIIHVKGKHLCPSPLFPGPSKPF |
| | GGACATCGCAACCTACTTCTGCCAGCAG | | WVLVVVGGVLACYSLLVTVAFIIFWVR |
| | GGGAACACCCTGCCCTACACCTTCGGTG | | SKRSRLLHSDYMNMTPRRPGPTRKHYQ |
| | GCGGGACCAAGCTGGAGATCACTGGCA | | PYAPPRDFAAYRSRVKFSRSADAPAYQQ |
| | GCACCTCAGGCTCTGGGAAGCCTGGCA | | GQNQLYNELNLGRREEYDVLDKRRGRD |
| | GCGGTGAAGGCAGCACCAAGGGTGAGG | | PEMGGKPRRKNPQEGLYNELQKDKMA |
| | TGAAGCTGCAGGAGTCCGGACCTGGTCT | | EAYSEIGMKGERRRGKGHDGLYQGLST |
| | GGTGGCCCCAAGCCAGTCCCTCAGCGTC | | ATKDTYDALHMQALPPR |
| | ACCTGCACAGTGTCCGGGGTGTCCCTGC | | |
| | CTGACTACGGTGTCTCCTGGATCAGGCA | | |
| | ACCACCCCGGAAGGGTCTCGAGTGGCT | | |
| | GGGCGTCATCTGGGGCTCCGAGACCAC | | |
| | CTACTACAACAGCGCTCTGAAGTCCCGG | | |
| | CTGACCATCATCAAAGACAACTCCAAG | | |
| | AGCCAGGTGTTCTTGAAGATGAACTCCC | | |
| | TGCAAACCGATGACACCGCCATCTACTA | | |
| | CTGCGCCAAGCACTACTACTATGGCGGT | | |
| | AGCTACGCCATGGATTATTGGGGTCAGG | | |
| | GCACCAGTGTCACCGTCTCCTCCGCTGC | | |
| | CGCTATCGAGGTGATGTACCCTCCACCC | | |
| | TATCTGGACAACGAGAAGTCCAACGGC | | |
| | ACCATCATCCACGTGAAGGGCAAGCAC | | |
| | CTGTGCCCTAGCCCTCTGTTCCCAGGAC | | |
| | CCTCCAAGCCCTTCTGGGTGCTGGTCGT | | |
| | GGTGGGAGGAGTCCTGGCCTGCTATTCC | | |
| | CTCCTCGTCACCGTGGCATTTATCATCTT | | |
| | CTGGGTCCGGAGCAAGCGGTCACGCCT | | |
| | GCTCCACTCCGACTACATGAACATGACT | | |
| | CCTCGCAGACCTGGACCCACCCGGAAG | | |
| | CACTACCAGCCTTATGCCCCACCCCGCG | | |
| | ACTTTGCCGCTTACCGCTCTCGGGTCAA | | |
| | GTTCTCTCGGTCAGCAGACGCCCCTGCA | | |
| | TACCAGCAGGGCCAGAACCAGCTGTAT | | |
| | AACGAGCTGAACCTCGGCAGACGGGAG | | |
| | GAGTACGATGTGCTGGACAAGAGGAGA | | |
| | GGCAGAGACCCCGAGATGGGTGGTAAG | | |
| | CCACGGCGCAAGAACCCACAGGAGGGC | | |
| | TTGTACAACGAACTGCAGAAGGACAAG | | |
| | ATGGCCGAGGCCTACAGCGAGATCGGC | | |
| | ATGAAGGGAGAGAGGCGCAGGGGCAA | | |
| | GGGTCACGACGGCCTGTACCAAGGGCT | | |
| | GTCCACCGCAACCAAGGACACCTACGA | | |
| | TGCCCTGCACATGCAGGCCCTCCCACCA | | |
| | AGG | | |
| 26 | ATGGCACTTCCAGTTACAGCACTTCTGC | 32 | MALPVTALLLPLALLLHAARPDIQMTQT |
| | TTCCATTGGCACTGCTGCTCCATGCAGC | | TSSLSASLGDRVTISCRASQDISKYLNWY |
| | TCGCCCCGATATCCAGATGACCCAGACC | | QQKPDGTVKLLIYHTSRLHSGVPSRFSG |
| | ACCTCTAGCCTCAGCGCCTCTCTGGGTG | | SGSGTDYSLTISNLEQEDIATYFCQQGNT |
| | ACCGCGTCACCATCTCTTGCCGGGCCAG | | LPYTFGGGTKLEITGSTSGSGKPGSGEGS |
| | CCAAGACATCTCTAAGTACCTGAACTGG | | TKGEVKLQESGPGLVAPSQSLSVTCTVS |
| | TACCAGCAGAAACCTGACGGAACCGTG | | GVSLPDYGVSWIRQPPRKGLEWLGVIW |
| | AAGCTGCTGATCTACCACACCAGTCGGC | | GSETTYYNSALKSRLTIIKDNSKSQVFLK |
| | TGCATTCCGGGGTGCCTTCCAGGTTCAG | | MNSLQTDDTAIYYCAKHYYYGGSYAM |
| | CGGTTCCGGCTCTGGGACCGATTATAGT | | DYWGQGTSVTVSSAAAIEVMYPPPYLD |
| | CTCACCATCTCCAACCTCGAGCAGGAGG | | NEKSNGTIIHVKGKHLCPSPLFPGPSKPF |
| | ACATCGCAACCTACTTCTGCCAGCAGGG | | WVLVVVGGVLACYSLLVTVAFIIFWVR |
| | GAACACCCTGCCCTACACCTTCGGTGGC | | SKRSRLLHSDYMNMTPRRPGPTRKHYQ |
| | GGGACCAAGCTGGAGATCACTGGCAGC | | PYAPPRDFAAYRSRVKFSRSADAPAYQQ |
| | ACCTCAGGCTCTGGGAAGCCTGGCAGC | | GQNQLYNELNLGRREEYDVLDKRRGRD |
| | GGTGAAGGCAGCACCAAGGGTGAGGTG | | PEMGGKPRRKNPQEGLYNELQKDKMA |
| | AAGCTGCAGGAGTCCGGACCTGGTCTG | | EAYSEIGMKGERRRGKGHDGLYQGLST |
| | GTGGCCCCAAGCCAGTCCCTCAGCGTCA | | ATKDTYDALHMQALPPR |
| | CCTGCACAGTGTCCGGGGTGTCCCTGCC | | |
| | TGACTACGGTGTCTCCTGGATCAGGCAA | | |
| | CCACCCCGGAAGGGTCTCGAGTGGCTG | | |
| | GGCGTCATCTGGGGCTCCGAGACCACCT | | |
| | ACTACAACAGCGCTCTGAAGTCCCGGCT | | |
| | GACCATCATCAAAGACAACTCCAAGAG | | |
| | CCAGGTGTTCTTGAAGATGAACTCCCTG | | |
| | CAAACCGATGACACCGCCATCTACTACT | | |
| | GCGCCAAGCACTACTACTATGGCGGTA | | |
| | GCTACGCCATGGATTATTGGGGTCAGGG | | |
| | CACCAGTGTCACCGTCTCCTCCGCTGCC | | |
| | GCTATCGAGGTGATGTACCCTCCACCCT | | |
| | ATCTGGACAACGAGAAGTCCAACGGCA | | |
| | CCATCATCCACGTGAAGGGCAAGCACC | | |
| | TGTGCCCTAGCCCTCTGTTCCCAGGACC | | |
| | CTCCAAGCCCTTCTGGGTGCTGGTCGTG | | |
| | GTGGGAGGAGTCCTGGCCTGCTATTCCC | | |
| | TCCTCGTCACCGTGGCATTTATCATCTTC | | |
| | TGGGTCCGGAGCAAGCGGTCACGCCTG | | |
| | CTCCACTCCGACTACATGAACATGACTC | | |
| | CTCGCAGACCTGGACCCACCCGGAAGC | | |
| | ACTACCAGCCTTATGCCCCACCCCGCGA | | |
| | CTTTGCCGCTTACCGCTCTCGGGTCAAG | | |
| | TTCTCTCGGTCAGCAGACGCCCCTGCAT | | |
| | ACCAGCAGGGCCAGAACCAGCTGTATA | | |
| | ACGAGCTGAACCTCGGCAGACGGGAGG | | |
| | AGTACGATGTGCTGGACAAGAGGAGAG | | |
| | GCAGAGACCCCGAGATGGGTGGTAAGC | | |
| | CACGGCGCAAGAACCCACAGGAGGGCT | | |
| | TGTACAACGAACTGCAGAAGGACAAGA | | |
| | TGGCCGAGGCCTACAGCGAGATCGGCA | | |
| | TGAAGGGAGAGAGGCGCAGGGGCAAG | | |
| | GGTCACGACGGCCTGTACCAAGGGCTG | | |
| | TCCACCGCAACCAAGGACACCTACGAT | | |
| | GCCCTGCACATGCAGGCCCTCCCACCAA | | |
| | GG | | |
| 27 | ATGGCACTGCCCGTCACCGCACTCCTGC | 33 | MALPVTALLLPLALLLHAARPDIQMTQT |
| | TCCCACTGGCACTGCTGCTCCATGCAGC | | TSSLSASLGDRVTISCRASQDISKYLNWY |
| | TCGCCCCGATATCCAGATGACCCAGACC | | QQKPDGTVKLLIYHTSRLHSGVPSRFSG |
| | ACCTCTAGCCTCAGCGCCTCTCTGGGTG | | SGSGTDYSLTISNLEQEDIATYFCQQGNT |
| | ACCGCGTCACCATCTCTTGCCGGGCCAG | | LPYTFGGGTKLEITGSTSGSGKPGSGEGS |
| | CCAAGACATCTCTAAGTACCTGAACTGG | | TKGEVKLQESGPGLVAPSQSLSVTCTVS |
| | TACCAGCAGAAACCTGACGGAACCGTG | | GVSLPDYGVSWIRQPPRKGLEWLGVIW |
| | AAGCTGCTGATCTACCACACCAGTCGGC | | GSETTYYNSALKSRLTIIKDNSKSQVFLK |
| | TGCATTCCGGGGTGCCTTCCAGGTTCAG | | MNSLQTDDTAIYYCAKHYYYGGSYAM |
| | CGGTTCCGGCTCTGGGACCGATTATAGT | | DYWGQGTSVTVSSAAAIEVMYPPPYLD |
| | CTCACCATCTCCAACCTCGAGCAGGAGG | | NEKSNGTIIHVKGKHLCPSPLFPGPSKPF |
| | ACATCGCAACCTACTTCTGCCAGCAGGG | | WVLVVVGGVLACYSLLVTVAFIIFWVR |
| | GAACACCCTGCCCTACACCTTCGGTGGC | | SKRSRLLHSDYMNMTPRRPGPTRKHYQ |
| | GGGACCAAGCTGGAGATCACTGGCAGC | | PYAPPRDFAAYRSRVKFSRSADAPAYQQ |
| | ACCTCAGGCTCTGGGAAGCCTGGCAGC | | GQNQLYNELNLGRREEYDVLDKRRGRD |
| | GGTGAAGGCAGCACCAAGGGTGAGGTG | | PEMGGKPRRKNPQEGLYNELQKDKMA |
| | AAGCTGCAGGAGTCCGGACCTGGTCTG | | EAYSEIGMKGERRRGKGHDGLYQGLST |
| | GTGGCCCCAAGCCAGTCCCTCAGCGTCA | | ATKDTYDALHMQALPPR |
| | CCTGCACAGTGTCCGGGGTGTCCCTGCC | | |
| | TGACTACGGTGTCTCCTGGATCAGGCAA | | |
| | CCACCCCGGAAGGGTCTCGAGTGGCTG | | |
| | GGCGTCATCTGGGGCTCCGAGACCACCT | | |
| | ACTACAACAGCGCTCTGAAGTCCCGGCT | | |
| | GACCATCATCAAAGACAACTCCAAGAG | | |
| | CCAGGTGTTCTTGAAGATGAACTCCCTG | | |
| | CAAACCGATGACACCGCCATCTACTACT | | |
| | GCGCCAAGCACTACTACTATGGCGGTA | | |
| | GCTACGCCATGGATTATTGGGGTCAGGG | | |
| | CACCAGTGTCACCGTCTCCTCCGCTGCC | | |
| | GCTATCGAGGTGATGTACCCTCCACCCT | | |
| | ATCTGGACAACGAGAAGTCCAACGGCA | | |
| | CCATCATCCACGTGAAGGGCAAGCACC | | |
| | TGTGCCCTAGCCCTCTGTTCCCAGGACC | | |
| | CTCCAAGCCCTTCTGGGTGCTGGTCGTG | | |
| | GTGGGAGGAGTCCTGGCCTGCTATTCCC | | |
| | TCCTCGTCACCGTGGCATTTATCATCTTC | | |
| | TGGGTCCGGAGCAAGCGGTCACGCCTG | | |
| | CTCCACTCCGACTACATGAACATGACTC | | |
| | CTCGCAGACCTGGACCCACCCGGAAGC | | |
| | ACTACCAGCCTTATGCCCCACCCCGCGA | | |
| | CTTTGCCGCTTACCGCTCTCGGGTCAAG | | |
| | TTCTCTCGGTCAGCAGACGCCCCTGCAT | | |
| | ACCAGCAGGGCCAGAACCAGCTGTATA | | |
| | ACGAGCTGAACCTCGGCAGACGGGAGG | | |
| | AGTACGATGTGCTGGACAAGAGGAGAG | | |
| | GCAGAGACCCCGAGATGGGTGGTAAGC | | |
| | CACGGCGCAAGAACCCACAGGAGGGCT | | |
| | TGTACAACGAACTGCAGAAGGACAAGA | | |
| | TGGCCGAGGCCTACAGCGAGATCGGCA | | |
| | TGAAGGGAGAGAGGCGCAGGGGCAAG | | |
| | GGTCACGACGGCCTGTACCAAGGGCTG | | |
| | TCCACCGCAACCAAGGACACCTACGAT | | |
| | GCCCTGCACATGCAGGCCCTCCCACCAA | | |
| | GG | | |
| 28 | ATGGCACTTCCAGTTACAGCACTTCTGC | 34 | MALPVTALLLPLALLLHAARPDIQMTQT |
| | TTCCATTGGCACTGCTGCTCCATGCAGC | | TSSLSASLGDRVTISCRASQDISKYLNWY |
| | TCGCCCCGATATCCAGATGACCCAGACC | | QQKPDGTVKLLIYHTSRLHSGVPSRFSG |
| | ACCTCTAGCCTCAGCGCCTCTCTGGGTG | | SGSGTDYSLTISNLEQEDIATYFCQQGNT |
| | ACCGCGTCACCATCTCTTGCCGGGCCAG | | LPYTFGGGTKLEITGGGGSGGGGSGGGG |
| | CCAAGACATCTCTAAGTACCTGAACTGG | | SEVKLQESGPGLVAPSQSLSVTCTVSGV |
| | TACCAGCAGAAACCTGACGGAACCGTG | | SLPDYGVSWIRQPPRKGLEWLGVIWGSE |
| | AAGCTGCTGATCTACCACACCAGTCGGC | | TTYYNSALKSRLTIIKDNSKSQVFLKMN |
| | TGCATTCCGGGGTGCCTTCCAGGTTCAG | | SLQTDDTAIYYCAKHYYYGGSYAMDY |
| | CGGTTCCGGCTCTGGGACCGATTATAGT | | WGQGTSVTVSSIEVMYPPPYLDNEKSNG |
| | CTCACCATCTCCAACCTCGAGCAGGAGG | | TIIHVKGKHLCPSPLFPGPSKPFWVLVVV |
| | ACATCGCAACCTACTTCTGCCAGCAGGG | | GGVLACYSLLVTVAFIIFWVRSKRSRLL |
| | GAACACCCTGCCCTACACCTTCGGTGGC | | HSDYMNMTPRRPGPTRKHYQPYAPPRD |
| | GGGACCAAGCTGGAGATCACTGGAGGT | | FAAYRSRVKFSRSADAPAYQQGQNQLY |
| | GGTGGCAGCGGAGGTGGAGGATCAGGT | | NELNLGRREEYDVLDKRRGRDPEMGGK |
| | GGAGGCGGTAGCGAGGTGAAGCTGCAG | | PRRKNPQEGLYNELQKDKMAEAYSEIG |
| | GAGTCCGGACCTGGTCTGGTGGCCCCAA | | MKGERRRGKGHDGLYQGLSTATKDTY |
| | GCCAGTCCCTCAGCGTCACCTGCACAGT | | DALHMQALPPR |
| | GTCCGGGGTGTCCCTGCCTGACTACGGT | | |
| | GTCTCCTGGATCAGGCAACCACCCCGGA | | |
| | AGGGTCTCGAGTGGCTGGGCGTCATCTG | | |
| | GGGCTCCGAGACCACCTACTACAACAG | | |
| | CGCTCTGAAGTCCCGGCTGACCATCATC | | |
| | AAAGACAACTCCAAGAGCCAGGTGTTC | | |
| | TTGAAGATGAACTCCCTGCAAACCGATG | | |
| | ACACCGCCATCTACTACTGCGCCAAGCA | | |
| | CTACTACTATGGCGGTAGCTACGCCATG | | |
| | GATTATTGGGGTCAGGGCACCAGTGTCA | | |
| | CCGTCTCCTCCATCGAGGTGATGTACCC | | |
| | TCCACCCTATCTGGACAACGAGAAGTCC | | |
| | AACGGCACCATCATCCACGTGAAGGGC | | |
| | AAGCACCTGTGCCCTAGCCCTCTGTTCC | | |
| | CAGGACCCTCCAAGCCCTTCTGGGTGCT | | |
| | GGTCGTGGTGGGAGGAGTCCTGGCCTG | | |
| | CTATTCCCTCCTCGTCACCGTGGCATTT | | |
| | ATCATCTTCTGGGTCCGGAGCAAGCGGT | | |
| | CACGCCTGCTCCACTCCGACTACATGAA | | |
| | CATGACTCCTCGCAGACCTGGACCCACC | | |
| | CGGAAGCACTACCAGCCTTATGCCCCAC | | |
| | CCCGCGACTTTGCCGCTTACCGCTCTCG | | |
| | GGTCAAGTTCTCTCGGTCAGCAGACGCC | | |
| | CCTGCATACCAGCAGGGCCAGAACCAG | | |
| | CTGTATAACGAGCTGAACCTCGGCAGA | | |
| | CGGGAGGAGTACGATGTGCTGGACAAG | | |
| | AGGAGAGGCAGAGACCCCGAGATGGGT | | |
| | GGTAAGCCACGGCGCAAGAACCCACAG | | |
| | GAGGGCTTGTACAACGAACTGCAGAAG | | |
| | GACAAGATGGCCGAGGCCTACAGCGAG | | |
| | ATCGGCATGAAGGGAGAGAGGCGCAGG | | |
| | GGCAAGGGTCACGACGGCCTGTACCAA | | |
| | GGGCTGTCCACCGCAACCAAGGACACC | | |
| | TACGATGCCCTGCACATGCAGGCCCTCC | | |
| | CACCAAGG | | |

Table 2C sets forth nucleotide and amino acid sequences for additional exemplary binder sequences that are not codon-optimized, including a mouse anti-CD19 binder, anti-BCMA binders, and anti-HER2 binders. In some embodiments, the circular RNA constructs and related pharmaceutical compositions disclosed herein comprise a CAR sequence encoding a polypeptide that comprises at least 80%, 85%, 90%, 91%, 92%, 93%, 94%, 95%, 96%, 97%, 98%, or 99% identity to any one of SEQ ID NO: 116-135 or binding fragments thereof.

In some embodiments, the circular RNA constructs and related pharmaceutical compositions disclosed herein comprise an IRES sequence having at least 85%, 86%, 87%, 88%, 89%, 90%, 91%, 92%, 93%, 94%, 95%, 96%, 97%, 98%, 99%, or 100% sequence identity to an IRES from Table 1A, an IRES from a construct of any one of SEQ ID NOs: 50-61 or any of Constructs A-P of Table 1B, and a CAR sequence encoding a polypeptide comprising at least 85%, 86%, 87%, 88%, 89%, 90%, 91%, 92%, 93%, 94%, 95%, 96%, 97%, 98%, 99%, or 100% sequence identity to any one of SEQ ID NO: 116-135 or binding fragments thereof. In some embodiments, said circular RNA further comprises a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

TABLE 2C
Additional Codon Amino Acid and Nucleotide Sequences

| | SEQ | | SEQ | |
|---|---|---|---|---|
| | ID | | ID | |
| | NO | Codon nucleotide sequence | NO | Codon amino acid sequence |
| Mouse | 100 | ATGGGCGTGCCTACCCAGCTGCTCGGTCT | 116 | MGVPTQLLGLLLLWITDAICDIQ |
| CD19 | | CCTGCTGCTCTGGATCACCGACGCTATCT | | MTQSPASLSTSLGETVTIQCQAS |
| | | GCGACATCCAAATGACCCAGAGTCCCGC | | EDIYSGLAWYQQKPGKSPQLLIY |
| | | TTCCCTCAGCACCTCCCTGGGTGAGACCG | | GASDLQDGVPSRFSGSGSGTQYS |
| | | TCACCATCCAGTGCCAGGCATCCGAGGA | | LKITSMQTEDEGVYFCQQGLTYP |
| | | CATCTACAGTGGTCTCGCCTGGTACCAGC | | RTFGGGTKLELKGGGGSGGGGS |
| | | AGAAGCCTGGTAAGTCCCCTCAGCTGCT | | GGGGSEVQLQQSGAELVRPGTS |
| | | GATCTACGGTGCTTCCGATCTGCAGGAC | | VKLSCKVSGDTITFYYMHFVKQ |
| | | GGAGTCCCTAGCCGCTTCTCAGGCTCTGG | | RPGQGLEWIGRIDPEDESTKYSE |
| | | CTCCGGTACCCAGTACTCCCTGAAGATC | | KFKNKATLTADTSSNTAYLKLSS |
| | | ACATCCATGCAGACCGAAGACGAGGGAG | | LTSEDTATYFCIYGGYYFDYWG |
| | | TGTACTTCTGCCAGCAGGGGCTGACCTA | | QGVMVTVSSIEFMYPPPYLDNER |
| | | CCCTCGGACATTCGGCGGTGGAACCAAG | | SNGTIIHIKEKHLCHTQSSPKLFW |
| | | CTCGAGCTGAAGGGAGGTGGAGGCAGTG | | ALVVVAGVLFCYGLLVTVALCV |
| | | GTGGCGGAGGATCTGGTGGTGGTGGCTC | | IWTNSRRNRGGQSDYMNMTPRR |
| | | CGAGGTCCAACTGCAGCAGTCCGGCGCT | | PGLTRKPYQPYAPARDFAAYRP |
| | | GAGCTGGTGAGGCCCGGAACCAGCGTCA | | RAKFSRSAETAANLQDPNQLYN |
| | | AACTCAGCTGCAAGGTGAGCGGGGACAC | | ELNLGRREEYDVLEKKRARDPE |
| | | CATCACCTTCTACTACATGCACTTCGTCA | | MGGKQQRRRNPQEGVYNALQK |
| | | AGCAGAGGCCTGGGCAGGGTCTTGAATG | | DKMAEAYSEIGTKGERRRGKGH |
| | | GATCGGCCGGATCGATCCAGAGGACGAG | | DGLYQGLSTATKDTYDALHMQT |
| | | TCTACAAAGTACTCCGAGAAGTTCAAGA | | LAPR |
| | | ACAAAGCAACCCTGACCGCCGACACAAG | | |
| | | CTCCAACACCGCCTACCTGAAGCTGTCC | | |
| | | AGCCTCACCTCTGAGGACACCGCCACCT | | |
| | | ACTTCTGCATCTACGGCGGGTACTACTTC | | |
| | | GACTATTGGGGCCAAGGGGTGATGGTCA | | |
| | | CCGTGTCCTCTATCGAGTTCATGTATCCT | | |
| | | CCTCCCTACCTGGACAACGAGCGGAGCA | | |
| | | ACGGCACCATCATCCACATCAAAGAGAA | | |
| | | GCACCTCTGCCACACCCAATCCTCTCCCA | | |
| | | AACTCTTCTGGGCCCTCGTTGTGGTCGCA | | |
| | | GGCGTGCTCTTCTGCTACGGGCTGCTGGT | | |
| | | GACTGTGGCCTTGTGCGTGATCTGGACC | | |
| | | AACAGTAGACGGAATCGGGGAGGTCAG | | |
| | | AGCGACTACATGAACATGACACCTCGCA | | |
| | | GACCAGGCCTGACACGGAAGCCCTACCA | | |
| | | ACCATACGCTCCTGCCCGGGATTTTGCAG | | |
| | | CATATCGGCCACGGGCCAAGTTTAGCAG | | |
| | | GTCCGCAGAGACCGCAGCCAACCTGCAA | | |
| | | GACCCTAACCAGCTGTACAACGAGCTGA | | |
| | | ACCTTGGTCGCCGGGAGGAGTACGACGT | | |
| | | CCTGGAGAAGAAGAGAGCACGGGATCCC | | |
| | | GAGATGGGCGGAAAGCAACAACGCCGG | | |
| | | CGGAATCCTCAGGAGGGTGTCTACAACG | | |
| | | CCCTCCAGAAGGACAAGATGGCTGAGGC | | |
| | | CTACTCCGAGATCGGCACTAAGGGCGAG | | |
| | | CGCAGACGGGGAAAGGGTCACGACGGG | | |
| | | CTGTACCAGGGTCTCAGCACCGCAACCA | | |
| | | AGGATACCTACGACGCCCTGCACATGCA | | |
| | | AACCCTCGCACCCCGG | | |
| HER2 | 101 | ATGGCTCTGCCTGTGACAGCTCTGCTGCT | 117 | MALPVTALLLPLALLLHAARPDI |
| | | GCCTCTGGCTCTGCTTCTGCATGCCGCCA | | QMTQSPSSLSASVGDRVTITCRA |
| | | GACCTGACATCCAGATGACTCAGAGCCC | | SQDVNTAVAWYQQKPGKAPKL |
| | | CAGCAGCCTGTCTGCCTCTGTGGGAGAC | | LIYSASFLYSGVPSRFSGSRSGTD |
| | | AGAGTGACAATTACCTGCCGGGCCAGCC | | FTLTISSLQPEDFATYYCQQHYT |
| | | AGGATGTGAATACTGCTGTCGCCTGGTA | | TPPTFGQGTKVEIKRTGSTSGSG |
| | | TCAACAAAAGCCTGGCAAGGCCCCTAAG | | KPGSGEGSEVQLVESGGGLVQP |
| | | CTCCTGATCTACAGCGCCAGCTTTCTGTA | | GGSLRLSCAASGFNIKDTYIHWV |
| | | CAGCGGCGTGCCCAGCAGATTCTCCGGA | | RQAPGKGLEWVARIYPTNGYTR |
| | | AGCAGAAGCGGCACAGATTTCACACTGA | | YADSVKGRFTISADTSKNTAYLQ |
| | | CCATAAGCAGCCTGCAGCCAGAGGATTT | | MNSLRAEDTAVYYCSRWGGDG |
| | | CGCCACCTACTATTGCCAGCAGCACTAC | | FYAMDVWGQGTLVTVSSIEVMY |
| | | ACCACACCTCCAACCTTTGGCCAGGGCA | | PPPYLDNEKSNGTIIHVKGKHLC |
| | | CCAAGGTCGAGATTAAGAGAACAGGCAG | | PSPLFPGPSKPFWVLVVVGGVLA |
| | | CACATCTGGCTCTGGCAAACCTGGATCT | | CYSLLVTVAFIIFWVRSKRSRLL |
| | | GGCGAGGGCTCTGAAGTCCAGCTGGTGG | | HSDYMNMTPRRPGPTRKHYQPY |
| | | AATCTGGCGGAGGACTGGTTCAACCTGG | | APPRDFAAYRSRVKFSRSADAPA |
| | | CGGCTCTCTGAGACTGTCTTGTGCCGCCT | | YQQGQNQLYNELNLGRREEYDV |
| | | CCGGCTTCAACATCAAGGACACCTACAT | | LDKRRGRDPEMGGKPRRKNPQE |
| | | CCACTGGGTCCGACAAGCCCCAGGCAAA | | GLYNELQKDKMAEAYSEIGMKG |
| | | GGACTTGAGTGGGTCGCCAGGATCTACC | | ERRRGKGHDGLYQGLSTATKDT |
| | | CCACCAACGGCTACACCAGATACGCCGA | | YDALHMQALPPR |
| | | CTCTGTGAAGGGCAGATTCACCATCTCTG | | |
| | | CCGACACCAGCAAGAATACCGCCTACCT | | |
| | | GCAGATGAACTCCCTGAGAGCCGAAGAT | | |
| | | ACCGCTGTGTATTACTGTTCCAGATGGGG | | |
| | | AGGCGACGGCTTCTACGCCATGGATGTT | | |
| | | TGGGGCCAAGGCACCCTCGTGACCGTTT | | |
| | | CTTCTATCGAAGTGATGTACCCTCCACCT | | |
| | | TACCTGGACAACGAGAAGTCCAACGGCA | | |
| | | CCATCATCCACGTGAAGGGCAAGCACCT | | |
| | | GTGTCCTTCTCCACTGTTCCCCGGACCTA | | |
| | | GCAAGCCTTTCTGGGTGCTCGTTGTTGTT | | |
| | | GGCGGCGTGCTGGCCTGTTACTCTCTGCT | | |
| | | GGTTACCGTGGCCTTCATCATCTTTTGGG | | |
| | | TCCGAAGCAAGCGGAGCCGGCTGCTGCA | | |
| | | CTCCGACTACATGAACATGACCCCTAGA | | |
| | | CGGCCCGGACCAACCAGAAAGCACTACC | | |
| | | AGCCTTACGCTCCTCCTAGAGACTTCGCC | | |
| | | GCCTACCGGTCCAGAGTGAAGTTCAGCA | | |
| | | GATCCGCCGATGCTCCCGCCTATCAGCA | | |
| | | GGGCCAAAACCAGCTGTACAACGAGCTG | | |
| | | AACCTGGGGAGAAGAGAAGAGTACGAC | | |
| | | GTGCTGGACAAGCGGAGAGGCAGAGATC | | |
| | | CTGAAATGGGCGGCAAGCCCAGACGGAA | | |
| | | GAATCCTCAAGAGGGCCTGTATAATGAG | | |
| | | CTGCAGAAAGACAAGATGGCCGAGGCCT | | |
| | | ACAGCGAGATCGGAATGAAGGGCGAGC | | |
| | | GCAGAAGAGGCAAGGGACACGATGGAC | | |
| | | TGTACCAGGGCCTGAGCACCGCCACCAA | | |
| | | GGATACCTATGATGCCCTGCACATGCAG | | |
| | | GCCCTGCCTCCAAGA | | |
| HER2 | 102 | ATGGCTCTGCCTGTGACAGCTCTGCTGCT | 118 | MALPVTALLLPLALLLHAARPDI |
| | | GCCTCTGGCTCTGCTTCTGCATGCCGCCA | | QMTQSPSSLSASVGDRVTITCRA |
| | | GACCTGACATCCAGATGACTCAGAGCCC | | SQDVNTAVAWYQQKPGKAPKL |
| | | CAGCAGCCTGTCTGCCTCTGTGGGAGAC | | LIYSASFLYSGVPSRFSGSRSGTD |
| | | AGAGTGACAATTACCTGCCGGGCCAGCC | | FTLTISSLQPEDFATYYCQQHYT |
| | | AGGATGTGAATACTGCTGTCGCCTGGTA | | TPPTFGQGTKVEIKRTGSTSGSG |
| | | TCAACAAAAGCCTGGCAAGGCCCCTAAG | | KPGSGEGSEVQLVESGGGLVQP |
| | | CTCCTGATCTACAGCGCCAGCTTTCTGTA | | GGSLRLSCAASGFNIKDTYIHWV |
| | | CAGCGGCGTGCCCAGCAGATTCTCCGGA | | RQAPGKGLEWVARIYPTNGYTR |
| | | AGCAGAAGCGGCACAGATTTCACACTGA | | YADSVKGRFTISADTSKNTAYLQ |
| | | CCATAAGCAGCCTGCAGCCAGAGGATTT | | MNSLRAEDTAVYYCSRWGGDG |
| | | CGCCACCTACTATTGCCAGCAGCACTAC | | FYAMDVWGQGTLVTVSSTTTPA |
| | | ACCACACCTCCAACCTTTGGCCAGGGCA | | PRPPTPAPTIASQPLSLRPEACRP |
| | | CCAAGGTCGAGATTAAGAGAACAGGCAG | | AAGGAVHTRGLDFACDIYIWAP |
| | | CACATCTGGCTCTGGCAAACCTGGATCT | | LAGTCGVLLLSLVITLYCKRGRK |
| | | GGCGAGGGCTCTGAAGTCCAGCTGGTGG | | KLLYIFKQPFMRPVQTTQEEDGC |
| | | AATCTGGCGGAGGACTGGTTCAACCTGG | | SCRFPEEEEGGCELRVKFSRSAD |
| | | CGGCTCTCTGAGACTGTCTTGTGCCGCCT | | APAYQQGQNQLYNELNLGRREE |
| | | CCGGCTTCAACATCAAGGACACCTACAT | | YDVLDKRRGRDPEMGGKPRRK |
| | | CCACTGGGTCCGACAAGCCCCAGGCAAA | | NPQEGLYNELQKDKMAEAYSEI |
| | | GGACTTGAGTGGGTCGCCAGGATCTACC | | GMKGERRRGKGHDGLYQGLST |
| | | CCACCAACGGCTACACCAGATACGCCGA | | ATKDTYDALHMQALPPR |
| | | CTCTGTGAAGGGCAGATTCACCATCTCTG | | |
| | | CCGACACCAGCAAGAATACCGCCTACCT | | |
| | | GCAGATGAACTCCCTGAGAGCCGAAGAT | | |
| | | ACCGCTGTGTATTACTGTTCCAGATGGGG | | |
| | | AGGCGACGGCTTCTACGCCATGGATGTT | | |
| | | TGGGGCCAAGGCACCCTCGTGACCGTTT | | |
| | | CTTCTACCACCACACCAGCTCCTCGGCCT | | |
| | | CCAACTCCTGCTCCTACAATTGCCAGCCA | | |
| | | GCCTCTGTCTCTGAGGCCCGAAGCTTGTA | | |
| | | GACCTGCTGCTGGCGGAGCCGTGCATAC | | |
| | | AAGAGGACTGGATTTCGCCTGCGACATC | | |
| | | TACATCTGGGCTCCTCTGGCCGGAACAT | | |
| | | GTGGCGTTCTGCTGCTGAGCCTGGTCATC | | |
| | | ACCCTGTACTGTAAGCGGGGCAGAAAGA | | |
| | | AGCTGCTGTACATCTTCAAGCAGCCCTTC | | |
| | | ATGCGGCCCGTGCAGACCACACAAGAGG | | |
| | | AAGATGGCTGCTCCTGCAGATTCCCCGA | | |
| | | GGAAGAAGAAGGCGGCTGCGAGCTGAG | | |
| | | AGTGAAGTTCAGCAGATCCGCCGATGCT | | |
| | | CCCGCCTATCAGCAGGGCCAAAACCAGC | | |
| | | TGTACAACGAGCTGAACCTGGGGAGAAG | | |
| | | AGAAGAGTACGACGTGCTGGACAAGCGG | | |
| | | AGAGGCAGAGATCCTGAAATGGGCGGCA | | |
| | | AGCCCAGACGGAAGAATCCTCAAGAGGG | | |
| | | CCTGTATAATGAGCTGCAGAAAGACAAG | | |
| | | ATGGCCGAGGCCTACAGCGAGATCGGAA | | |
| | | TGAAGGGCGAGCGCAGAAGAGGCAAGG | | |
| | | GACACGATGGACTGTACCAGGGCCTGAG | | |
| | | CACCGCCACCAAGGATACCTATGATGCC | | |
| | | CTGCACATGCAGGCCCTGCCTCCAAGA | | |
| BCMA | 103 | ATGGCACTCCCGGTAACCGCCTTATTGCT | 119 | MALPVTALLLPLALLLHAARPDI |
| | | TCCCCTTGCCCTCTTGCTCCACGCAGCAC | | VLTQSPPSLAMSLGKRATISCRA |
| | | GCCCCGATATAGTCTTGACTCAATCCCCA | | SESVTILGSHLIHWYQQKPGQPP |
| | | CCCAGTTTGGCAATGTCATTAGGCAAAC | | TLLIQLASNVQTGVPARFSGSGS |
| | | GAGCAACAATTTCATGTAGGGCATCCGA | | RTDFTLTIDPVEEDDVAVYYCLQ |
| | | AAGTGTAACGATTTTGGGGAGTCATTTA | | SRTIPRTFGGGTKLEIKGSTSGSG |
| | | ATTCATTGGTACCAACAAAAGCCTGGAC | | KPGSGEGSTKGQIQLVQSGPELK |
| | | AACCCCCGACGCTCTTGATCCAATTAGC | | KPGETVKISCKASGYTFTDYSIN |
| | | ATCTAACGTCCAAACCGGAGTCCCCGCA | | WVKRAPGKGLKWMGWINTETR |
| | | CGATTCTCAGGATCCGGTTCCCGGACTG | | EPAYAYDFRGRFAFSLETSASTA |
| | | ATTTTACATTAACTATTGATCCGGTAGAG | | YLQINNLKYEDTATYFCALDYS |
| | | GAAGATGACGTCGCTGTCTATTATTGTCT | | YAMDYWGQGTSVTVSSAAATT |
| | | TCAAAGTAGGACGATTCCACGGACATTC | | TPAPRPPTPAPTIASQPLSLRPEA |
| | | GGTGGCGGAACTAAATTGGAGATTAAAG | | CRPAAGGAVHTRGLDFACDIYI |
| | | GTTCCACCTCTGGTAGTGGGAAACCCGG | | WAPLAGTCGVLLLSLVITLYCKR |
| | | GTCCGGTGAAGGGTCCACTAAAGGCCAA | | GRKKLLYIFKQPFMRPVQTTQEE |
| | | ATTCAACTCGTTCAATCCGGACCAGAAC | | DGCCRFPEEEEGGCELRVKFSR |
| | | TGAAGAAGCCAGGAGAAACTGTCAAAAT | | SADAPAYQQGQNQLYNELNLGR |
| | | AAGCTGTAAAGCTTCCGGTTATACATTTA | | REEYDVLDKRRGRDPEMGGKPR |
| | | CAGATTATTCCATAAATTGGGTGAAAAG | | RKNPQEGLYNELQKDKMAEAYS |
| | | GGCGCCAGGAAAAGGGTTAAAGTGGATG | | EIGMKGERRRGKGHDGLYQGLS |
| | | GGTTGGATTAATACAGAGACTCGGGAAC | | TATKDTYDALHMQALPPR |
| | | CTGCATATGCTTATGATTTTAGGGGAAG | | |
| | | GTTTGCCTTTTCTCTGGAGACTTCCGCTT | | |
| | | CAACTGCTTATCTCCAAATTAATAATCTT | | |
| | | AAATATGAGGACACAGCAACATACTTCT | | |
| | | GTGCTTTGGACTATAGTTATGCTATGGAT | | |
| | | TACTGGGGACAAGGAACCAGTGTCACTG | | |
| | | TAAGTTCCGCTGCTGCGACGACCACTCCT | | |
| | | GCACCGCGACCACCCACTCCTGCCCCTA | | |
| | | CTATTGCTAGTCAACCACTTAGCTTGCGA | | |
| | | CCTGAGGCATGTCGGCCCGCGGCAGGTG | | |
| | | GCGCAGTCCACACCAGGGGTTTAGACTT | | |
| | | TGCTTGTGATATTTATATTTGGGCACCAC | | |
| | | TCGCCGGGACTTGCGGTGTTCTTCTCTTG | | |
| | | TCCCTTGTTATAACTCTTTATTGTAAGCG | | |
| | | CGGAAGGAAGAAATTGTTATATATTTTC | | |
| | | AAACAACCTTTTATGCGACCCGTACAAA | | |
| | | CAACTCAGGAAGAGGACGGGTGTTCTTG | | |
| | | TCGGTTTCCAGAAGAGGAAGAGGGTGGG | | |
| | | TGTGAACTCCGGGTCAAATTTAGTAGGT | | |
| | | CAGCAGATGCGCCGGCGTACCAACAAGG | | |
| | | CCAAAACCAACTGTATAATGAACTCAAT | | |
| | | CTCGGTAGGCGTGAGGAATATGATGTCC | | |
| | | TTGATAAAAGGCGCGGGAGAGATCCAGA | | |
| | | AATGGGCGGAAAACCACGGCGAAAGAA | | |
| | | TCCGCAGGAAGGGTTATATAACGAACTT | | |
| | | CAAAAGGATAAAATGGCTGAAGCTTATT | | |
| | | CCGAAATTGGCATGAAAGGAGAGCGACG | | |
| | | TAGGGGCAAAGGGCATGATGGCCTTTAC | | |
| | | CAAGGGCTCTCAACCGCTACAAAAGATA | | |
| | | CTTACGACGCTTTACATATGCAAGCACTT | | |
| | | CCACCCAGG | | |
| BCMA | 104 | ATGGCCCTCCCCGTCACAGCTCTCCTGCT | 120 | MALPVTALLLPLALLLHAARPDI |
| | | CCCACTGGCCCTTCTTTTGCACGCTGCTC | | VLTQSPPSLAMSLGKRATISCRA |
| | | GCCCCGATATCGTGCTCACCCAGTCACCT | | SESVTILGSHLIHWYQQKPGQPP |
| | | CCAAGCCTTGCCATGAGCCTCGGGAAAC | | TLLIQLASNVQTGVPARFSGSGS |
| | | GGGCTACCATCTCCTGCCGGGCTTCAGA | | RTDFTLTIDPVEEDDVAVYYCLQ |
| | | GTCCGTCACCATCCTCGGGTCACACCTCA | | SRTIPRTFGGGTKLEIKGSTSGSG |
| | | TCCACTGGTACCAACAGAAACCAGGGCA | | KPGSGEGSTKGQIQLVQSGPELK |
| | | GCCTCCTACCCTCTTGATCCAGTTGGCCT | | KPGETVKISCKASGYTFTDYSIN |
| | | CCAACGTGCAAACTGGGGTTCCCGCCAG | | WVKRAPGKGLKWMGWINTETR |
| | | GTTCAGTGGCTCCGGATCCCGGACAGAT | | EPAYAYDERGRFAFSLETSASTA |
| | | TTCACACTTACCATCGATCCTGTGGAGGA | | YLQINNLKYEDTATYFCALDYS |
| | | GGACGATGTGGCCGTCTATTACTGCCTGC | | YAMDYWGQGTSVTVSSAAATT |
| | | AGTCTCGCACCATCCCTCGGACCTTCGGT | | TPAPRPPTPAPTIASQPLSLRPEA |
| | | GGAGGCACCAAGCTCGAGATCAAGGGTA | | CRPAAGGAVHTRGLDFACDIYI |
| | | GCACCTCCGGCTCTGGAAAGCCAGGCTC | | WAPLAGTCGVLLLSLVITLYCRS |
| | | TGGTGAGGGTTCTACCAAGGGCCAAATC | | KRSRLLHSDYMNMTPRRPGPTR |
| | | CAGCTGGTCCAGTCTGGGCCCGAGCTGA | | KHYQPYAPPRDFAAYRSRVKFS |
| | | AGAAACCCGGGGAGACCGTGAAGATCTC | | RSADAPAYQQGQNQLYNELNLG |
| | | CTGCAAGGCCTCCGGTTATACCTTCACCG | | RREEYDVLDKRRGRDPEMGGKP |
| | | ACTACTCCATCAACTGGGTCAAGCGCGC | | RRKNPQEGLYNELQKDKMAEA |
| | | TCCTGGAAAGGGCCTCAAGTGGATGGGC | | YSEIGMKGERRRGKGHDGLYQG |
| | | TGGATCAACACCGAAACCCGCGAGCCTG | | LSTATKDTYDALHMQALPPR |
| | | CCTATGCTTACGACTTCAGGGGCCGGTTC | | |
| | | GCTTTCTCACTGGAGACCTCCGCTTCCAC | | |
| | | AGCCTACCTCCAGATCAACAACCTCAAG | | |
| | | TACGAAGACACCGCCACCTATTTCTGCG | | |
| | | CTCTCGACTATTCCTACGCTATGGACTAC | | |
| | | TGGGGTCAGGGCACCTCTGTGACCGTCT | | |
| | | CTAGCGCAGCCGCCACCACAACACCAGC | | |
| | | CCCACGGCCACCTACTCCCGCACCCACC | | |
| | | ATCGCATCCCAACCACTCAGTCTGAGGC | | |
| | | CCGAGGCCTGTAGACCTGCTGCTGGAGG | | |
| | | CGCAGTGCATACCCGCGGTCTCGACTTC | | |
| | | GCCTGCGACATCTATATCTGGGCCCCATT | | |
| | | GGCAGGTACCTGTGGCGTGCTGCTGCTG | | |
| | | TCACTCGTCATCACCCTGTACTGCCGGAG | | |
| | | TAAGCGCTCTAGGCTGTTGCACAGCGAC | | |
| | | TACATGAACATGACCCCAAGAAGACCAG | | |
| | | GGCCTACCCGGAAGCACTACCAGCCATA | | |
| | | CGCACCTCCCCGGGACTTTGCCGCCTATC | | |
| | | GGTCTCGGGTGAAGTTCTCACGCTCCGCT | | |
| | | GATGCCCCAGCATACCAGCAGGGGCAGA | | |
| | | ACCAGCTGTACAATGAGCTCAACCTCGG | | |
| | | TCGCCGCGAAGAGTACGACGTGCTCGAC | | |
| | | AAGAGAAGGGGCAGGGACCCTGAGATG | | |
| | | GGAGGCAAGCCCCGCAGAAAGAATCCCC | | |
| | | AGGAAGGTCTGTACAACGAGCTGCAAAA | | |
| | | GGATAAGATGGCTGAGGCCTACAGCGAG | | |
| | | ATCGGCATGAAGGGCGAAAGGAGACGG | | |
| | | GGAAAGGGCCACGACGGGCTCTACCAGG | | |
| | | GACTCTCCACCGCCACCAAGGACACCTA | | |
| | | CGACGCCCTCCACATGCAGGCTCTGCCA | | |
| | | CCCAGG | | |
| BCMA | 105 | ATGGCACTTCCCGTCACCGCTCTCCTGCT | 121 | MALPVTALLLPLALLLHAARPDI |
| | | GCCCCTCGCACTGCTGCTCCATGCAGCCC | | VLTQSPPSLAMSLGKRATISCRA |
| | | GCCCAGACATCGTCCTGACCCAGTCCCCT | | SESVTILGSHLIHWYQQKPGQPP |
| | | CCCCCCTCGCAATGTCCCTCGGGAAAC | | TLLIQLASNVQTGVPARFSGSGS |
| | | GGGCCACCATCAGCTGCCGGGCCTCTGA | | RTDFTLTIDPVEEDDVAVYYCLQ |
| | | GTCAGTGACAATCCTCGGAAGCCATCTG | | SRTIPRTFGGGTKLEIKGSTSGSG |
| | | ATCCATTGGTACCAGCAGAAACCCGGTC | | KPGSGEGSTKGQIQLVQSGPELK |
| | | AGCCTCCAACCCTCCTCATCCAGCTGGCC | | KPGETVKISCKASGYTFTDYSIN |
| | | TCCAACGTGCAGACAGGAGTCCCCGCTC | | WVKRAPGKGLKWMGWINTETR |
| | | GGTTCTCAGGCAGCGGTTCCAGGACCGA | | EPAYAYDFRGRFAFSLETSASTA |
| | | CTTCACCCTGACCATCGACCCCGTGGAA | | YLQINNLKYEDTATYFCALDYS |
| | | GAGGACGATGTGGCTGTGTACTACTGCC | | YAMDYWGQGTSVTVSSIEVMYP |
| | | TCCAGTCCCGGACCATCCCACGGACCTTC | | PPYLDNEKSNGTIIHVKGKHLCP |
| | | GGAGGTGGGACAAAGCTGGAGATCAAA | | SPLFPGPSKPFWVLVVVGGVLAC |
| | | GGCAGCACCAGCGGTTCTGGCAAGCCAG | | YSLLVTVAFIIFWVRSKRSRLLHS |
| | | GGTCAGGTGAGGGGAGCACAAAGGGTC | | DYMNMTPRRPGPTRKHYQPYAP |
| | | AGATCCAGCTGGTGCAGAGCGGTCCCGA | | PRDFAAYRSRVKFSRSADAPAY |
| | | GCTGAAGAAGCCCGGGGAGACCGTTAAG | | QQGQNQLYNELNLGRREEYDVL |
| | | ATCTCCTGCAAGGCTAGCGGGTACACCT | | DKRRGRDPEMGGKPRRKNPQEG |
| | | TCACCGACTATAGTATCAACTGGGTCAA | | LYNELQKDKMAEAYSEIGMKGE |
| | | GCGCGCTCCTGGCAAGGGGCTCAAGTGG | | RRRGKGHDGLYQGLSTATKDTY |
| | | ATGGGGTGGATCAACACCGAAACCAGGG | | DALHMQALPPR |
| | | AGCCCGCATACGCTTATGACTTTCGGGG | | |
| | | CCGGTTCGCCTTTTCCCTGGAGACCAGCG | | |
| | | CCTCTACCGCCTACCTCCAGATCAACAAC | | |
| | | CTGAAGTACGAGGACACCGCCACCTACT | | |
| | | TCTGCGCACTCGACTACTCCTACGCTATG | | |
| | | GACTACTGGGGTCAGGGTACCTCCGTCA | | |
| | | CCGTCTCCAGCATCGAGGTCATGTACCCT | | |
| | | CCTCCCTACCTGGACAACGAGAAGTCCA | | |
| | | ACGGCACCATCATCCATGTGAAGGGCAA | | |
| | | GCATCTCTGCCCCAGCCCACTGTTCCCCG | | |
| | | GACCCTCTAAGCCCTTCTGGGTCCTGGTC | | |
| | | GTCGTCGGCGGTGTTCTGGCTTGCTACAG | | |
| | | CTTGCTGGTCACCGTCGCCTTCATCATCT | | |
| | | TCTGGGTGCGCTCCAAGAGGAGCCGGCT | | |
| | | GCTGCATAGCGACTACATGAACATGACC | | |
| | | CCTAGAAGGCCTGGTCCAACCCGCAAGC | | |
| | | ACTACCAGCCTTACGCCCCTCCACGGGA | | |
| | | CTTCGCAGCCTACCGGTCACGGGTGAAG | | |
| | | TTCTCTCGGAGCGCAGATGCCCCAGCAT | | |
| | | ACCAGCAGGGCCAGAACCAGCTGTACAA | | |
| | | CGAACTTAACCTTGGTCGGCGGGAGGAA | | |
| | | TACGATGTGCTGGACAAGCGCAGGGGTC | | |
| | | GGGATCCTGAAATGGGCGGGAAACCACG | | |
| | | CCGGAAGAACCCACAGGAGGGGCTCTAT | | |
| | | AACGAGCTCCAAAAGGATAAGATGGCTG | | |
| | | AGGCTTACAGCGAGATTGGAATGAAGGG | | |
| | | AGAAAGAAGACGGGGCAAGGGTCACGA | | |
| | | CGGGTTGTACCAGGGTCTGAGCACCGCC | | |
| | | ACCAAGGACACCTACGACGCCCTCCACA | | |
| | | TGCAAGCCCTTCCACCCCGC | | |
| BCMA | 106 | ATGGCTCTCCCCGTGACCGCTCTGCTGCT | 122 | MALPVTALLLPLALLLHAARPQ |
| | | CCCTCTGGCCCTCCTTCTGCACGCAGCCA | | VKLEESGGGLVQAGRSLRLSCA |
| | | GACCACAGGTCAAGCTGGAGGAGTCTGG | | ASEHTFSSHVMGWFRQAPGKER |
| | | TGGCGGTCTGGTGCAGGCAGGGAGGAGC | | ESVAVIGWRDISTSYADSVKGRF |
| | | CTGAGGCTGAGCTGTGCAGCTTCCGAGC | | TISRDNAKKTLYLQMNSLKPEDT |
| | | ACACATTCTCAAGCCACGTCATGGGGTG | | AVYYCAARRIDAADFDSWGQGT |
| | | GTTCAGACAGGCTCCCGGTAAAGAGAGG | | QVTVSSGGGGSGGGGSGGGGSE |
| | | GAGTCCGTCGCCGTGATCGGATGGCGGG | | VQLVESGGGLVQAGGSLRLSCA |
| | | ACATCTCCACCTCCTACGCCGACTCTGTG | | ASGRTFTMGWFRQAPGKEREFV |
| | | AAGGGCCGGTTCACAATCTCACGCGATA | | AAISLSPTLAYYAESVKGRFTISR |
| | | ATGCCAAGAAGACACTGTATCTGCAGAT | | DNAKNTVVLQMNSLKPEDTALY |
| | | GAATTCCTTGAAGCCCGAAGACACCGCC | | YCAADRKSVMSIRPDYWGQGTQ |
| | | GTCTATTACTGTGCTGCTAGACGGATCGA | | VTVSSTSTTTPAPRPPTPAPTIAS |
| | | CGCTGCCGACTTCGACAGCTGGGGACAG | | QPLSLRPEACRPAAGGAVHTRG |
| | | GGTACCCAAGTGACCGTTTCCTCCGGAG | | LDFACDIYIWAPLAGTCGVLLLS |
| | | GCGGAGGTTCTGGAGGAGGTGGGTCAGG | | LVITLYCKRGRKKLLYIFKQPFM |
| | | TGGAGGTGGCTCCGAGGTGCAGCTGGTC | | RPVQTTQEEDGCSCRFPEEEEGG |
| | | GAGTCTGGCGGTGGCTTGGTCCAGGCTG | | CELRVKFSRSADAPAYQQGQNQ |
| | | GAGGCAGTCTCAGACTCTCCTGCGCTGCT | | LYNELNLGRREEYDVLDKRRGR |
| | | TCAGGGCGGACCTTCACCATGGGCTGGT | | DPEMGGKPRRKNPQEGLYNELQ |
| | | TCAGGCAGGCCCCAGGTAAGGAGAGGG | | KDKMAEAYSEIGMKGERRRGK |
| | | AGTTCGTGGCCGCCATCTCCCTCTCCCCT | | GHDGLYQGLSTATKDTYDALH |
| | | ACCCTGGCATACTACGCTGAGTCCGTGA | | MQALPPR |
| | | AGGGACGGTTTACCATCTCCCGGGATAA | | |
| | | CGCAAAGAACACTGTGGTCCTCCAAATG | | |
| | | AACTCCCTCAAACCCGAGGACACCGCTC | | |
| | | TCTACTATTGTGCCGCAGATCGGAAGAG | | |
| | | CGTCATGTCCATCCGGCCCGATTACTGGG | | |
| | | GCCAAGGCACACAGGTGACTGTGTCCAG | | |
| | | CACCTCCACCACCACCCCAGCACCAAGG | | |
| | | CCTCCAACCCCTGCACCAACCATCGCCTC | | |
| | | CCAGCCACTGTCTTTGCGGCCAGAAGCA | | |
| | | TGCCGCCCAGCAGCAGGTGGAGCCGTGC | | |
| | | ATACAAGAGGCCTGGACTTCGCCTGCGA | | |
| | | TATCTACATCTGGGCTCCTCTGGCCGGAA | | |
| | | CATGCGGAGTCCTGCTCTTGTCCCTGGTG | | |
| | | ATCACCCTGTACTGCAAGCGGGGTCGGA | | |
| | | AGAAGCTCCTCTACATCTTCAAGCAGCC | | |
| | | CTTCATGAGACCCGTCCAGACCACCCAG | | |
| | | GAGGAGGACGGGTGCTCATGCAGGTTCC | | |
| | | CCGAAGAGGAGGAGGGTGGCTGTGAGCT | | |
| | | GCGGGTGAAGTTCAGCAGGTCAGCAGAC | | |
| | | GCCCCTGCCTATCAGCAGGGCCAAAACC | | |
| | | AGTTGTACAACGAGCTGAATCTGGGGAG | | |
| | | ACGGGAGGAGTACGATGTCCTTGACAAG | | |
| | | AGAAGGGGCCGGGATCCAGAGATGGGC | | |
| | | GGGAAGCCAAGACGGAAGAATCCTCAG | | |
| | | GAGGGTCTGTATAACGAGCTGCAGAAGG | | |
| | | ACAAGATGGCCGAGGCCTACTCCGAGAT | | |
| | | CGGCATGAAAGGGGAGCGCCGCAGAGG | | |
| | | AAAAGGTCACGATGGTCTGTACCAGGGG | | |
| | | TTGAGCACCGCTACCAAGGATACTTACG | | |
| | | ACGCTCTGCACATGCAAGCTCTGCCACC | | |
| | | CCGG | | |
| BCMA | 107 | ATGGCACTCCCCGTTACCGCCCTTCTGCT | 123 | MALPVTALLLPLALLLHAARPQ |
| | | GCCTCTCGCCTTGCTGCTGCACGCAGCCA | | VKLEESGGGLVQAGRSLRLSCA |
| | | GACCACAGGTCAAGCTGGAGGAGTCTGG | | ASEHTFSSHVMGWFRQAPGKER |
| | | TGGCGGGCTCGTTCAAGCAGGTCGGAGT | | ESVAVIGWRDISTSYADSVKGRF |
| | | CTCCGCCTGTCTTGCGCAGCATCAGAGC | | TISRDNAKKTLYLQMNSLKPEDT |
| | | ATACCTTCTCCTCACACGTGATGGGGTGG | | AVYYCAARRIDAADFDSWGQGT |
| | | TTCAGGCAAGCTCCCGGTAAGGAGAGGG | | QVTVSSGGGGSGGGGSGGGGSE |
| | | AGTCCGTGGCCGTTATCGGCTGGCGCGA | | VQLVESGGGLVQAGGSLRLSCA |
| | | TATCAGCACCTCCTACGCAGACAGCGTT | | ASGRTFTMGWFRQAPGKEREFV |
| | | AAGGGCCGGTTCACTATCTCCAGGGACA | | AAISLSPTLAYYAESVKGRFTISR |
| | | ACGCTAAGAAGACACTCTACCTCCAGAT | | DNAKNTVVLQMNSLKPEDTALY |
| | | GAACAGTCTGAAGCCCGAGGACACCGCA | | YCAADRKSVMSIRPDYWGQGTQ |
| | | GTGTACTATTGCGCTGCTCGGCGGATCG | | VTVSSTSTTTPAPRPPTPAPTIAS |
| | | ATGCTGCCGACTTCGACAGCTGGGGTCA | | QPLSLRPEACRPAAGGAVHTRG |
| | | AGGGACCCAGGTCACCGTTTCCAGCGGA | | LDFACDIYIWAPLAGTCGVLLLS |
| | | GGTGGCGGAAGTGGTGGCGGAGGATCAG | | LVITLYCRSKRSRLLHSDYMNM |
| | | GTGGTGGAGGCTCCGAGGTCCAGCTGGT | | TPRRPGPTRKHYQPYAPPRDFAA |
| | | GGAATCAGGAGGCGGCTTGGTGCAGGCT | | YRSRVKFSRSADAPAYQQGQNQ |
| | | GGTGGGTCTTTGCGGTTGTCCTGCGCAGC | | LYNELNLGRREEYDVLDKRRGR |
| | | TTCCGGCAGGACCTTCACCATGGGATGG | | DPEMGGKPRRKNPQEGLYNELQ |
| | | TTCAGACAAGCCCCAGGTAAGGAGCGGG | | KDKMAEAYSEIGMKGERRRGK |
| | | AGTTTGTGGCCGCAATCTCACTGTCTCCC | | GHDGLYQGLSTATKDTYDALH |
| | | ACCCTCGCTTACTACGCCGAGAGTGTGA | | MQALPPR |
| | | AGGGGCGCTTCACAATCAGTCGCGACAA | | |
| | | CGCAAAGAACACCGTCGTCCTGCAAATG | | |
| | | AACTCCCTGAAGCCTGAGGATACCGCAC | | |
| | | TCTATTACTGCGCCGCCGATCGGAAGAG | | |
| | | CGTCATGTCCATCCGGCCCGACTATTGGG | | |
| | | GCCAAGGCACCCAAGTGACCGTCAGCTC | | |
| | | CACCTCCACAACCACTCCCGCCCCAAGA | | |
| | | CCACCTACCCCAGCCCCAACAATCGCAT | | |
| | | CCCAGCCTCTGTCCCTTCGGCCCGAAGCT | | |
| | | TGTCGCCCTGCAGCAGGTGGAGCAGTGC | | |
| | | ACACCCGGGGACTGGACTTCGCCTGCGA | | |
| | | CATCTACATCTGGGCACCCCTGGCTGGA | | |
| | | ACCTGCGGCGTGTTGCTGCTGAGCCTGGT | | |
| | | GATCACCCTCTACTGCCGCTCTAAGAGA | | |
| | | AGCCGGCTGCTGCATAGCGACTACATGA | | |
| | | ACATGACCCCTAGGAGACCAGGACCCAC | | |
| | | CCGGAAGCACTACCAGCCTTACGCTCCT | | |
| | | CCACGGGATTTCGCTGCTTACCGCAGCC | | |
| | | GGGTGAAGTTTTCCAGGTCAGCTGACGC | | |
| | | CCCTGCCTACCAGCAGGGCCAGAACCAA | | |
| | | TTGTACAACGAACTGAATCTGGGACGGC | | |
| | | GCGAGGAATACGACGTCCTGGACAAGAG | | |
| | | GCGGGGTAGAGATCCCGAGATGGGCGGG | | |
| | | AAACCTCGGCGGAAGAACCCTCAGGAGG | | |
| | | GGCTCTACAACGAGCTGCAGAAGGATAA | | |
| | | GATGGCCGAAGCCTACTCCGAGATCGGG | | |
| | | ATGAAGGGTGAACGGAGGAGGGGCAAG | | |
| | | GGACACGACGGCCTGTATCAGGGCCTCA | | |
| | | GCACCGCTACCAAGGACACCTACGACGC | | |
| | | CCTGCACATGCAGGCTCTCCCACCACGG | | |
| BCMA | 108 | ATGGCTCTTCCCGTCACCGCTTTGCTGCT | 124 | MALPVTALLLPLALLLHAARPQ |
| | | GCCCCTGGCACTCCTCCTCCATGCTGCTC | | VKLEESGGGLVQAGRSLRLSCA |
| | | GGCCTCAGGTGAAGCTGGAGGAGAGTGG | | ASEHTFSSHVMGWFRQAPGKER |
| | | TGGCGGTCTGGTGCAAGCTGGCAGATCT | | ESVAVIGWRDISTSYADSVKGRF |
| | | CTGCGCCTGTCTTGCGCAGCCAGCGAAC | | TISRDNAKKTLYLQMNSLKPEDT |
| | | ACACCTTCTCCTCCCACGTGATGGGGTGG | | AVYYCAARRIDAADFDSWGQGT |
| | | TTTCGGCAGGCACCCGGGAAAGAGCGCG | | QVTVSSGGGGSGGGGSGGGGSE |
| | | AGTCCGTCGCAGTCATCGGGTGGCGGGA | | VQLVESGGGLVQAGGSLRLSCA |
| | | CATCTCTACCAGCTACGCAGATTCCGTCA | | ASGRTFTMGWFRQAPGKEREFV |
| | | AGGGCCGGTTCACCATTTCCCGGGATAA | | AAISLSPTLAYYAESVKGRFTISR |
| | | CGCTAAGAAGACCCTCTACCTGCAAATG | | DNAKNTVVLQMNSLKPEDTALY |
| | | AACTCTCTGAAGCCCGAAGACACCGCCG | | YCAADRKSVMSIRPDYWGQGTQ |
| | | TCTACTATTGCGCAGCAAGGCGCATCGA | | VTVSSIEVMYPPPYLDNEKSNGT |
| | | CGCTGCCGACTTCGACTCTTGGGGCCAA | | IIHVKGKHLCPSPLFPGPSKPFWV |
| | | GGAACCCAGGTCACCGTGTCTTCCGGAG | | LVVVGGVLACYSLLVTVAFIIFW |
| | | GAGGAGGCTCCGGTGGTGGAGGTTCTGG | | VRSKRSRLLHSDYMNMTPRRPG |
| | | AGGTGGCGGCTCAGAGGTGCAGCTCGTG | | PTRKHYQPYAPPRDFAAYRSRV |
| | | GAGAGCGGTGGTGGACTCGTTCAGGCAG | | KFSRSADAPAYQQGQNQLYNEL |
| | | GCGGCAGTTTGCGGCTGTCCTGTGCAGC | | NLGRREEYDVLDKRRGRDPEMG |
| | | CTCCGGTCGCACTTTCACTATGGGATGGT | | GKPRRKNPQEGLYNELQKDKM |
| | | TCCGCCAGGCTCCTGGTAAAGAAAGGGA | | AEAYSEIGMKGERRRGKGHDGL |
| | | GTTCGTGGCCGCCATCAGTCTGAGCCCC | | YQGLSTATKDTYDALHMQALPP |
| | | ACCCTCGCATACTACGCCGAGAGCGTGA | | R |
| | | AGGGTAGGTTCACTATCAGCCGGGACAA | | |
| | | CGCCAAGAACACCGTGGTGCTCCAGATG | | |
| | | AATTCCCTGAAGCCTGAGGATACCGCCC | | |
| | | TCTACTACTGCGCTGCCGACCGCAAGAG | | |
| | | CGTGATGAGCATCCGGCCTGACTATTGG | | |
| | | GGTCAGGGGACACAGGTGACCGTCAGCA | | |
| | | GCATCGAGGTGATGTATCCACCACCCTA | | |
| | | CCTCGACAACGAGAAGTCCAACGGCACC | | |
| | | ATCATCCACGTCAAGGGGAAGCACCTCT | | |
| | | GCCCTTCCCCTCTGTTCCCTGGCCCCTCA | | |
| | | AAGCCCTTCTGGGTCCTGGTGGTGGTTGG | | |
| | | TGGGGTGCTGGCTTGCTACTCCCTGCTCG | | |
| | | TGACCGTGGCTTTCATCATCTTCTGGGTT | | |
| | | CGGAGCAAACGGTCCAGACTGCTGCACT | | |
| | | CCGACTACATGAACATGACCCCAAGAAG | | |
| | | ACCTGGGCCCACACGGAAGCATTACCAA | | |
| | | CCCTATGCACCACCTCGGGATTTCGCCGC | | |
| | | CTACAGATCCCGGGTCAAGTTCTCCAGG | | |
| | | TCCGCCGATGCACCAGCCTATCAGCAGG | | |
| | | GGCAAAACCAGCTGTATAATGAGCTGAA | | |
| | | CCTTGGACGGCGCGAGGAGTACGACGTG | | |
| | | CTCGACAAAAGACGCGGTCGCGACCCAG | | |
| | | AGATGGGCGGCAAGCCTAGACGCAAGA | | |
| | | ATCCCCAGGAGGGGCTCTATAACGAGTT | | |
| | | GCAGAAGGATAAGATGGCCGAGGCCTAC | | |
| | | AGCGAGATCGGGATGAAAGGCGAAAGA | | |
| | | CGGCGCGGAAAGGGTCACGACGGACTCT | | |
| | | ACCAGGGCCTGAGCACAGCCACCAAAGA | | |
| | | CACCTACGACGCTCTGCATATGCAAGCA | | |
| | | CTGCCTCCCCGG | | |
| BCMA | 109 | ATGGCTCTCCCCGTGACCGCTCTCCTCCT | 125 | MALPVTALLLPLALLLHAARPQ |
| | | TCCCTTGGCCTTGTTGCTGCACGCCGCAA | | VKLEESGGGLVQAGRSLRLSCA |
| | | GGCCTCAGGTGAAGCTGGAGGAATCCGG | | ASEHTFSSHVMGWFRQAPGKER |
| | | AGGCGGACTCGTCCAAGCAGGTCGGTCC | | ESVAVIGWRDISTSYADSVKGRF |
| | | CTCAGGCTGTCTTGCGCTGCCAGCGAGC | | TISRDNAKKTLYLQMNSLKPEDT |
| | | ACACCTTCTCTAGCCACGTGATGGGTTGG | | AVYYCAARRIDAADFDSWGQGT |
| | | TTCAGACAGGCCCCAGGAAAGGAGCGGG | | QVTVSSTSTTTPAPRPPTPAPTIA |
| | | AATCCGTGGCAGTGATCGGCTGGCGGGA | | SQPLSLRPEACRPAAGGAVHTRG |
| | | CATCAGCACCTCCTACGCCGACTCCGTCA | | LDFACDIYIWAPLAGTCGVLLLS |
| | | AGGGCCGGTTCACCATCAGCCGCGACAA | | LVITLYCKRGRKKLLYIFKQPFM |
| | | CGCCAAGAAGACCCTGTACCTGCAGATG | | RPVQTTQEEDGCSCRFPEEEEGG |
| | | AACAGCCTCAAGCCTGAGGACACCGCCG | | CELRVKFSRSADAPAYQQGQNQ |
| | | TGTACTACTGCGCCGCCAGAAGGATCGA | | LYNELNLGRREEYDVLDKRRGR |
| | | CGCCGCAGACTTCGACTCCTGGGGTCAG | | DPEMGGKPRRKNPQEGLYNELQ |
| | | GGAACCCAGGTGACCGTGTCCTCCACCT | | KDKMAEAYSEIGMKGERRRGK |
| | | CTACCACCACACCAGCACCCAGACCTCC | | GHDGLYQGLSTATKDTYDALH |
| | | TACTCCCGCTCCCACCATCGCTTCCCAGC | | MQALPPR |
| | | CCCTGTCCCTCAGACCCGAAGCCTGCAG | | |
| | | ACCAGCAGCTGGCGGTGCAGTGCACACC | | |
| | | AGGGGTCTTGACTTCGCCTGTGACATCTA | | |
| | | CATCTGGGCTCCACTGGCTGGGACTTGC | | |
| | | GGCGTTCTGCTGCTGAGCCTGGTGATCAC | | |
| | | CCTGTACTGCAAGCGGGGCCGGAAGAAG | | |
| | | CTGCTCTACATCTTCAAGCAGCCTTTCAT | | |
| | | GCGGCCCGTTCAGACCACCCAGGAGGAA | | |
| | | GACGGGTGCAGTTGCCGCTTCCCTGAGG | | |
| | | AGGAGGAGGGAGGATGCGAGCTGCGGG | | |
| | | TCAAGTTCTCTCGGTCCGCTGATGCCCCA | | |
| | | GCCTACCAGCAGGGCCAGAACCAGCTCT | | |
| | | ATAACGAGCTGAACCTCGGTAGGCGGGA | | |
| | | GGAGTACGACGTCCTGGACAAAAGGAGG | | |
| | | GGACGGGATCCTGAGATGGGAGGAAAG | | |
| | | CCACGGCGGAAGAACCCTCAAGAGGGGC | | |
| | | TGTACAACGAACTCCAGAAGGACAAGAT | | |
| | | GGCTGAGGCTTATTCTGAGATCGGCATG | | |
| | | AAGGGAGAGCGCAGACGCGGCAAGGGA | | |
| | | CACGATGGCCTGTATCAGGGACTGAGCA | | |
| | | CCGCCACCAAGGATACCTACGACGCCCT | | |
| | | CCATATGCAGGCTCTCCCACCACGG | | |
| BCMA | 110 | ATGGCACTGCCCGTCACAGCTCTGCTGCT | 126 | MALPVTALLLPLALLLHAARPEV |
| | | GCCCTTGGCCCTCTTGTTGCACGCAGCAC | | QLVESGGGLVQAGGSLRLSCAA |
| | | GGCCAGAGGTGCAACTTGTCGAGAGCGG | | SGRTFTMGWFRQAPGKEREFVA |
| | | TGGTGGCCTTGTGCAGGCCGGAGGCAGT | | AISLSPTLAYYAESVKGRFTISRD |
| | | TTGCGCCTCAGTTGTGCAGCTTCTGGCCG | | NAKNTVVLQMNSLKPEDTALYY |
| | | GACCTTCACCATGGGCTGGTTCAGACAG | | CAADRKSVMSIRPDYWGQGTQV |
| | | GCCCCTGGTAAGGAACGCGAGTTCGTGG | | TVSSTSTTTPAPRPPTPAPTIASQP |
| | | CCGCCATCAGCCTGTCCCCAACCCTGGCC | | LSLRPEACRPAAGGAVHTRGLD |
| | | TACTACGCAGAGAGCGTGAAGGGTCGGT | | FACDIYIWAPLAGTCGVLLLSLVI |
| | | TCACCATCTCCCGCGACAACGCAAAGAA | | TLYCKRGRKKLLYIFKQPFMRPV |
| | | CACCGTGGTGCTCCAGATGAACTCCCTG | | QTTQEEDGCSCRFPEEEEGGCEL |
| | | AAGCCAGAAGACACCGCCCTGTACTACT | | RVKFSRSADAPAYQQGQNQLYN |
| | | GCGCAGCCGACCGGAAGAGCGTGATGTC | | ELNLGRREEYDVLDKRRGRDPE |
| | | CATCCGCCCTGACTACTGGGGCCAAGGC | | MGGKPRRKNPQEGLYNELQKD |
| | | ACACAGGTCACAGTGTCCAGCACCTCCA | | KMAEAYSEIGMKGERRRGKGH |
| | | CTACCACTCCAGCTCCACGCCCTCCAACA | | DGLYQGLSTATKDTYDALHMQ |
| | | CCCGCACCAACCATCGCCAGCCAGCCTC | | ALPPR |
| | | TGAGTCTGAGACCCGAAGCATGCCGGCC | | |
| | | AGCTGCTGGAGGTGCCGTGCACACCAGA | | |
| | | GGGCTGGACTTCGCCTGCGACATCTACA | | |
| | | TCTGGGCTCCTCTGGCCGGAACTTGCGG | | |
| | | GGTGCTGCTCCTCTCACTGGTCATCACCC | | |
| | | TGTACTGCAAGAGGGGCAGGAAGAAGCT | | |
| | | CCTGTACATCTTCAAGCAGCCCTTCATGC | | |
| | | GGCCAGTCCAGACAACCCAGGAGGAAG | | |
| | | ACGGATGCAGCTGTCGCTTCCCCGAGGA | | |
| | | GGAGGAAGGCGGCTGCGAATTGCGGGTC | | |
| | | AAGTTCAGCAGATCCGCTGACGCTCCTG | | |
| | | CCTACCAACAGGGACAGAACCAGCTCTA | | |
| | | CAACGAGCTGAACCTGGGAAGGCGGGA | | |
| | | GGAGTACGACGTCCTGGACAAGAGAAGA | | |
| | | GGACGCGACCCCGAGATGGGAGGTAAGC | | |
| | | CCAGACGCAAGAACCCTCAAGAGGGACT | | |
| | | GTATAACGAGCTGCAGAAGGACAAGATG | | |
| | | GCCGAGGCCTACAGCGAGATCGGCATGA | | |
| | | AGGGTGAAAGAAGACGGGGAAAGGGGC | | |
| | | ACGACGGTCTGTATCAGGGGCTCTCCAC | | |
| | | CGCAACCAAGGATACCTATGACGCTCTG | | |
| | | CACATGCAGGCACTCCCTCCACGC | | |
| BCMA | 111 | ATGGCACTGCCCGTGACCGCTCTGCTGCT | 127 | MALPVTALLLPLALLLHAARPQ |
| | | GCCTCTCGCCCTGCTGCTGCATGCTGCCA | | VKLEESGGGLVQAGRSLRLSCA |
| | | GGCCCCAAGTCAAGCTCGAGGAGTCTGG | | ASEHTFSSHVMGWFRQAPGKER |
| | | TGGTGGGCTCGTCCAAGCTGGCAGGTCC | | ESVAVIGWRDISTSYADSVKGRF |
| | | CTGAGACTGAGTTGTGCCGCCTCCGAGC | | TISRDNAKKTLYLQMNSLKPEDT |
| | | ACACTTTCAGCTCTCACGTGATGGGCTGG | | AVYYCAARRIDAADFDSWGQGT |
| | | TTCAGGCAGGCACCCGGTAAGGAGCGGG | | QVTVSSIEVMYPPPYLDNEKSNG |
| | | AGTCTGTGGCTGTTATCGGGTGGCGGGA | | TIIHVKGKHLCPSPLFPGPSKPFW |
| | | TATCTCTACCTCCTACGCCGACTCCGTCA | | VLVVVGGVLACYSLLVTVAFIIF |
| | | AGGGCCGGTTCACCATCTCCAGGGACAA | | WVRSKRSRLLHSDYMNMTPRRP |
| | | CGCAAAGAAGACCCTCTACCTCCAGATG | | GPTRKHYQPYAPPRDFAAYRSR |
| | | AACTCACTCAAGCCCGAGGACACCGCTG | | VKFSRSADAPAYQQGQNQLYNE |
| | | TGTACTACTGCGCAGCCAGACGCATCGA | | LNLGRREEYDVLDKRRGRDPEM |
| | | TGCCGCAGACTTCGACTCCTGGGGCCAA | | GGKPRRKNPQEGLYNELQKDK |
| | | GGTACCCAAGTGACAGTGTCCAGCATCG | | MAEAYSEIGMKGERRRGKGHD |
| | | AGGTGATGTACCCACCTCCCTACCTCGAC | | GLYQGLSTATKDTYDALHMQAL |
| | | AACGAGAAGAGCAACGGCACCATCATCC | | PPR |
| | | ACGTGAAGGGGAAGCACCTGTGTCCCTC | | |
| | | TCCCCTTTTCCCAGGACCCTCCAAGCCAT | | |
| | | TCTGGGTCCTGGTCGTTGTCGGAGGCGTG | | |
| | | CTCGCTTGCTATTCCCTGCTCGTCACCGT | | |
| | | GGCCTTCATCATCTTCTGGGTGCGGTCCA | | |
| | | AGAGATCCCGGCTGCTGCACTCTGATTA | | |
| | | CATGAACATGACACCCAGGAGGCCAGGG | | |
| | | CCTACCAGGAAGCACTACCAGCCCTACG | | |
| | | CTCCTCCACGCGACTTCGCAGCATACCG | | |
| | | GTCCCGCGTCAAGTTCTCCCGGTCCGCAG | | |
| | | ATGCTCCAGCCTATCAGCAGGGCCAGAA | | |
| | | CCAGCTGTACAACGAACTCAATTTGGGG | | |
| | | AGGCGCGAGGAGTATGATGTGCTCGATA | | |
| | | AGAGAAGGGGCCGGGATCCTGAGATGG | | |
| | | GAGGCAAGCCCAGACGGAAGAACCCTCA | | |
| | | GGAGGGCCTGTACAATGAGCTGCAGAAG | | |
| | | GACAAGATGGCCGAGGCCTACTCCGAGA | | |
| | | TCGGGATGAAGGGTGAAAGAAGGAGGG | | |
| | | GTAAGGGGCACGACGGGCTCTACCAAGG | | |
| | | CCTGAGCACCGCTACCAAGGACACCTAC | | |
| | | GATGCACTGCATATGCAAGCCCTGCCAC | | |
| | | CACGG | | |
| BCMA | 112 | ATGGCCCTCCCTGTCACAGCCCTCTTGCT | 128 | MALPVTALLLPLALLLHAARPEV |
| | | GCCCCTCGCACTCCTTCTGCACGCAGCTC | | QLVESGGGLVQAGGSLRLSCAA |
| | | GGCCTGAGGTCCAGCTGGTCGAGAGTGG | | SGRTFTMGWFRQAPGKEREFVA |
| | | AGGCGGACTGGTCCAGGCAGGTGGGAGT | | AISLSPTLAYYAESVKGRFTISRD |
| | | CTTCGGCTTTCCTGCGCAGCTTCCGGACG | | NAKNTVVLQMNSLKPEDTALYY |
| | | GACCTTCACCATGGGTTGGTTCCGGCAG | | CAADRKSVMSIRPDYWGQGTQV |
| | | GCACCTGGGAAGGAGAGGGAGTTCGTTG | | TVSSIEVMYPPPYLDNEKSNGTII |
| | | CCGCTATCAGCCTCTCACCAACCCTGGCC | | HVKGKHLCPSPLFPGPSKPFWVL |
| | | TACTATGCAGAGAGCGTCAAGGGCCGCT | | VVVGGVLACYSLLVTVAFIIFWV |
| | | TCACCATCAGCCGCGACAACGCCAAGAA | | RSKRSRLLHSDYMNMTPRRPGP |
| | | CACCGTCGTGCTGCAGATGAACTCCCTC | | TRKHYQPYAPPRDFAAYRSRVK |
| | | AAGCCCGAGGATACCGCCCTGTACTACT | | FSRSADAPAYQQGQNQLYNELN |
| | | GCGCTGCCGATCGGAAGTCCGTCATGTC | | LGRREEYDVLDKRRGRDPEMGG |
| | | CATTCGGCCCGACTACTGGGGACAGGGC | | KPRRKNPQEGLYNELQKDKMAE |
| | | ACACAGGTGACCGTCAGCAGCATCGAGG | | AYSEIGMKGERRRGKGHDGLYQ |
| | | TCATGTACCCTCCACCCTACCTGGACAAC | | GLSTATKDTYDALHMQALPPR |
| | | GAGAAGAGCAACGGGACCATCATCCACG | | |
| | | TGAAGGGGAAGCACCTCTGTCCAAGTCC | | |
| | | CCTCTTCCCAGGACCCTCCAAGCCATTCT | | |
| | | GGGTCCTCGTGGTGGTTGGAGGAGTGCT | | |
| | | CGCCTGCTACTCTCTGCTGGTGACCGTCG | | |
| | | CCTTCATCATCTTCTGGGTGCGGTCCAAG | | |
| | | CGGTCTCGCCTCCTCCACTCCGACTACAT | | |
| | | GAACATGACACCACGCAGACCTGGGCCC | | |
| | | ACTAGGAAGCACTATCAGCCCTATGCAC | | |
| | | CACCCCGGGATTTCGCAGCCTACCGGTC | | |
| | | ACGGGTGAAGTTCAGCAGATCCGCAGAC | | |
| | | GCACCAGCCTACCAGCAGGGGCAGAACC | | |
| | | AGCTGTATAACGAGCTGAACCTCGGTCG | | |
| | | CAGGGAGGAGTACGATGTCCTGGATAAG | | |
| | | AGAAGGGGCAGGGATCCCGAGATGGGT | | |
| | | GGCAAGCCCAGACGGAAGAATCCTCAGG | | |
| | | AGGGGCTCTACAACGAGCTGCAGAAGGA | | |
| | | CAAGATGGCCGAGGCTTACTCAGAGATC | | |
| | | GGCATGAAAGGGGAGAGGAGGCGCGGA | | |
| | | AAAGGCCACGACGGCCTCTACCAGGGAC | | |
| | | TGTCCACCGCAACCAAGGATACCTACGA | | |
| | | CGCCCTGCACATGCAAGCCCTCCCACCTC | | |
| | | GG | | |
| BCMA | 113 | ATGGCTCTCCCCGTGACCGCTCTCCTCCT | 129 | MALPVTALLLPLALLLHAARPQ |
| | | TCCCTTGGCCTTGTTGCTGCACGCCGCAA | | VKLEESGGGLVQAGRSLRLSCA |
| | | GGCCTCAGGTGAAGCTGGAGGAATCCGG | | ASEHTFSSHVMGWFRQAPGKER |
| | | AGGCGGACTCGTCCAAGCAGGTCGGTCC | | ESVAVIGWRDISTSYADSVKGRF |
| | | CTCAGGCTGTCTTGCGCTGCCAGCGAGC | | TISRDNAKKTLYLQMNSLKPEDT |
| | | ACACCTTCTCTAGCCACGTGATGGGTTGG | | AVYYCAARRIDAADFDSWGQGT |
| | | TTCAGACAGGCCCCAGGAAAGGAGCGGG | | QVTVSSTSTTTPAPRPPTPAPTIA |
| | | AATCCGTGGCAGTGATCGGCTGGCGGGA | | SQPLSLRPEACRPAAGGAVHTRG |
| | | CATCAGCACCTCCTACGCCGACTCCGTCA | | LDFACDIYIWAPLAGTCGVLLLS |
| | | AGGGCCGGTTCACCATCAGCCGCGACAA | | LVITLYCKRGRKKLLYIFKQPFM |
| | | CGCCAAGAAGACCCTGTACCTGCAGATG | | RPVQTTQEEDGCSCRFPEEEEGG |
| | | AACAGCCTCAAGCCTGAGGACACCGCCG | | CELRVKFSRSADAPAYQQGQNQ |
| | | TGTACTACTGCGCCGCCAGAAGGATCGA | | LYNELNLGRREEYDVLDKRRGR |
| | | CGCCGCAGACTTCGACTCCTGGGGTCAG | | DPEMGGKPRRKNPQEGLYNELQ |
| | | GGAACCCAGGTGACCGTGTCCTCCACCT | | KDKMAEAYSEIGMKGERRRGK |
| | | CTACCACCACACCAGCACCCAGACCTCC | | GHDGLYQGLSTATKDTYDALH |
| | | TACTCCCGCTCCCACCATCGCTTCCCAGC | | MQALPPRGSGEGRGSLLTCGDV |
| | | CCCTGTCCCTCAGACCCGAAGCCTGCAG | | EENPGPMALPVTALLLPLALLLH |
| | | ACCAGCAGCTGGCGGTGCAGTGCACACC | | AARPEVQLVESGGGLVQAGGSL |
| | | AGGGGTCTTGACTTCGCCTGTGACATCTA | | RLSCAASGRTFTMGWFRQAPGK |
| | | CATCTGGGCTCCACTGGCTGGGACTTGC | | EREFVAAISLSPTLAYYAESVKG |
| | | GGCGTTCTGCTGCTGAGCCTGGTGATCAC | | RFTISRDNAKNTVVLQMNSLKPE |
| | | CCTGTACTGCAAGCGGGGCCGGAAGAAG | | DTALYYCAADRKSVMSIRPDYW |
| | | CTGCTCTACATCTTCAAGCAGCCTTTCAT | | GQGTQVTVSSIEVMYPPPYLDNE |
| | | GCGGCCCGTTCAGACCACCCAGGAGGAA | | KSNGTIIHVKGKHLCPSPLFPGPS |
| | | GACGGGTGCAGTTGCCGCTTCCCTGAGG | | KPFWVLVVVGGVLACYSLLVTV |
| | | AGGAGGAGGGAGGATGCGAGCTGCGGG | | AFIIFWVRSKRSRLLHSDYMNMT |
| | | TCAAGTTCTCTCGGTCCGCTGATGCCCCA | | PRRPGPTRKHYQPYAPPRDFAAY |
| | | GCCTACCAGCAGGGCCAGAACCAGCTCT | | RSRVKFSRSADAPAYQQGQNQL |
| | | ATAACGAGCTGAACCTCGGTAGGCGGGA | | YNELNLGRREEYDVLDKRRGRD |
| | | GGAGTACGACGTCCTGGACAAAAGGAGG | | PEMGGKPRRKNPQEGLYNELQK |
| | | GGACGGGATCCTGAGATGGGAGGAAAG | | DKMAEAYSEIGMKGERRRGKG |
| | | CCACGGCGGAAGAACCCTCAAGAGGGGC | | HDGLYQGLSTATKDTYDALHM |
| | | TGTACAACGAACTCCAGAAGGACAAGAT | | QALPPR |
| | | GGCTGAGGCTTATTCTGAGATCGGCATG | | |
| | | AAGGGAGAGCGCAGACGCGGCAAGGGA | | |
| | | CACGATGGCCTGTATCAGGGACTGAGCA | | |
| | | CCGCCACCAAGGATACCTACGACGCCCT | | |
| | | CCATATGCAGGCTCTCCCACCACGGGGA | | |
| | | TCTGGCGAAGGCAGAGGATCTCTGCTGA | | |
| | | CATGCGGCGACGTGGAAGAGAACCCTGG | | |
| | | ACCTATGGCCCTCCCTGTCACAGCCCTCT | | |
| | | TGCTGCCCCTCGCACTCCTTCTGCACGCA | | |
| | | GCTCGGCCTGAGGTCCAGCTGGTCGAGA | | |
| | | GTGGAGGCGGACTGGTCCAGGCAGGTGG | | |
| | | GAGTCTTCGGCTTTCCTGCGCAGCTTCCG | | |
| | | GACGGACCTTCACCATGGGTTGGTTCCG | | |
| | | GCAGGCACCTGGGAAGGAGAGGGAGTTC | | |
| | | GTTGCCGCTATCAGCCTCTCACCAACCCT | | |
| | | GGCCTACTATGCAGAGAGCGTCAAGGGC | | |
| | | CGCTTCACCATCAGCCGCGACAACGCCA | | |
| | | AGAACACCGTCGTGCTGCAGATGAACTC | | |
| | | CCTCAAGCCCGAGGATACCGCCCTGTAC | | |
| | | TACTGCGCTGCCGATCGGAAGTCCGTCA | | |
| | | TGTCCATTCGGCCCGACTACTGGGGACA | | |
| | | GGGCACACAGGTGACCGTCAGCAGCATC | | |
| | | GAGGTCATGTACCCTCCACCCTACCTGG | | |
| | | ACAACGAGAAGAGCAACGGGACCATCAT | | |
| | | CCACGTGAAGGGGAAGCACCTCTGTCCA | | |
| | | AGTCCCCTCTTCCCAGGACCCTCCAAGCC | | |
| | | ATTCTGGGTCCTCGTGGTGGTTGGAGGA | | |
| | | GTGCTCGCCTGCTACTCTCTGCTGGTGAC | | |
| | | CGTCGCCTTCATCATCTTCTGGGTGCGGT | | |
| | | CCAAGCGGTCTCGCCTCCTCCACTCCGAC | | |
| | | TACATGAACATGACACCACGCAGACCTG | | |
| | | GGCCCACTAGGAAGCACTATCAGCCCTA | | |
| | | TGCACCACCCCGGGATTTCGCAGCCTAC | | |
| | | CGGTCACGGGTGAAGTTCAGCAGATCCG | | |
| | | CAGACGCACCAGCCTACCAGCAGGGGCA | | |
| | | GAACCAGCTGTATAACGAGCTGAACCTC | | |
| | | GGTCGCAGGGAGGAGTACGATGTCCTGG | | |
| | | ATAAGAGAAGGGGCAGGGATCCCGAGA | | |
| | | TGGGTGGCAAGCCCAGACGGAAGAATCC | | |
| | | TCAGGAGGGGCTCTACAACGAGCTGCAG | | |
| | | AAGGACAAGATGGCCGAGGCTTACTCAG | | |
| | | AGATCGGCATGAAAGGGGAGAGGAGGC | | |
| | | GCGGAAAAGGCCACGACGGCCTCTACCA | | |
| | | GGGACTGTCCACCGCAACCAAGGATACC | | |
| | | TACGACGCCCTGCACATGCAAGCCCTCC | | |
| | | CACCTCGG | | |
| BCMA | 114 | ATGGCACTGCCCGTCACAGCTCTGCTGCT | 130 | MALPVTALLLPLALLLHAARPEV |
| | | GCCCTTGGCCCTCTTGTTGCACGCAGCAC | | QLVESGGGLVQAGGSLRLSCAA |
| | | GGCCAGAGGTGCAACTTGTCGAGAGCGG | | SGRTFTMGWFRQAPGKEREFVA |
| | | TGGTGGCCTTGTGCAGGCCGGAGGCAGT | | AISLSPTLAYYAESVKGRFTISRD |
| | | TTGCGCCTCAGTTGTGCAGCTTCTGGCCG | | NAKNTVVLQMNSLKPEDTALYY |
| | | GACCTTCACCATGGGCTGGTTCAGACAG | | CAADRKSVMSIRPDYWGQGTQV |
| | | GCCCCTGGTAAGGAACGCGAGTTCGTGG | | TVSSTSTTTPAPRPPTPAPTIASQP |
| | | CCGCCATCAGCCTGTCCCCAACCCTGGCC | | LSLRPEACRPAAGGAVHTRGLD |
| | | TACTACGCAGAGAGCGTGAAGGGTCGGT | | FACDIYIWAPLAGTCGVLLLSLVI |
| | | TCACCATCTCCCGCGACAACGCAAAGAA | | TLYCKRGRKKLLYIFKQPFMRPV |
| | | CACCGTGGTGCTCCAGATGAACTCCCTG | | QTTQEEDGCSCRFPEEEEGGCEL |
| | | AAGCCAGAAGACACCGCCCTGTACTACT | | RVKFSRSADAPAYQQGQNQLYN |
| | | GCGCAGCCGACCGGAAGAGCGTGATGTC | | ELNLGRREEYDVLDKRRGRDPE |
| | | CATCCGCCCTGACTACTGGGGCCAAGGC | | MGGKPRRKNPQEGLYNELQKD |
| | | ACACAGGTCACAGTGTCCAGCACCTCCA | | KMAEAYSEIGMKGERRRGKGH |
| | | CTACCACTCCAGCTCCACGCCCTCCAACA | | DGLYQGLSTATKDTYDALHMQ |
| | | CCCGCACCAACCATCGCCAGCCAGCCTC | | ALPPRGSGEGRGSLLTCGDVEEN |
| | | TGAGTCTGAGACCCGAAGCATGCCGGCC | | PGPMALPVTALLLPLALLLHAAR |
| | | AGCTGCTGGAGGTGCCGTGCACACCAGA | | PQVKLEESGGGLVQAGRSLRLSC |
| | | GGGCTGGACTTCGCCTGCGACATCTACA | | AASEHTFSSHVMGWFRQAPGKE |
| | | TCTGGGCTCCTCTGGCCGGAACTTGCGG | | RESVAVIGWRDISTSYADSVKGR |
| | | GGTGCTGCTCCTCTCACTGGTCATCACCC | | FTISRDNAKKTLYLQMNSLKPED |
| | | TGTACTGCAAGAGGGGCAGGAAGAAGCT | | TAVYYCAARRIDAADFDSWGQG |
| | | CCTGTACATCTTCAAGCAGCCCTTCATGC | | TQVTVSSIEVMYPPPYLDNEKSN |
| | | GGCCAGTCCAGACAACCCAGGAGGAAG | | GTIIHVKGKHLCPSPLFPGPSKPF |
| | | ACGGATGCAGCTGTCGCTTCCCCGAGGA | | WVLVVVGGVLACYSLLVTVAFII |
| | | GGAGGAAGGCGGCTGCGAATTGCGGGTC | | FWVRSKRSRLLHSDYMNMTPRR |
| | | AAGTTCAGCAGATCCGCTGACGCTCCTG | | PGPTRKHYQPYAPPRDFAAYRSR |
| | | CCTACCAACAGGGACAGAACCAGCTCTA | | VKFSRSADAPAYQQGQNQLYNE |
| | | CAACGAGCTGAACCTGGGAAGGCGGGA | | LNLGRREEYDVLDKRRGRDPEM |
| | | GGAGTACGACGTCCTGGACAAGAGAAGA | | GGKPRRKNPQEGLYNELQKDK |
| | | GGACGCGACCCCGAGATGGGAGGTAAGC | | MAEAYSEIGMKGERRRGKGHD |
| | | CCAGACGCAAGAACCCTCAAGAGGGACT | | GLYQGLSTATKDTYDALHMQAL |
| | | GTATAACGAGCTGCAGAAGGACAAGATG | | PPR |
| | | GCCGAGGCCTACAGCGAGATCGGCATGA | | |
| | | AGGGTGAAAGAAGACGGGGAAAGGGGC | | |
| | | ACGACGGTCTGTATCAGGGGCTCTCCAC | | |
| | | CGCAACCAAGGATACCTATGACGCTCTG | | |
| | | CACATGCAGGCACTCCCTCCACGCGGAT | | |
| | | CTGGCGAAGGCAGAGGATCTCTGCTGAC | | |
| | | ATGCGGCGACGTGGAAGAGAACCCTGGA | | |
| | | CCTATGGCACTGCCCGTGACCGCTCTGCT | | |
| | | GCTGCCTCTCGCCCTGCTGCTGCATGCTG | | |
| | | CCAGGCCCCAAGTCAAGCTCGAGGAGTC | | |
| | | TGGTGGTGGGCTCGTCCAAGCTGGCAGG | | |
| | | TCCCTGAGACTGAGTTGTGCCGCCTCCGA | | |
| | | GCACACTTTCAGCTCTCACGTGATGGGCT | | |
| | | GGTTCAGGCAGGCACCCGGTAAGGAGCG | | |
| | | GGAGTCTGTGGCTGTTATCGGGTGGCGG | | |
| | | GATATCTCTACCTCCTACGCCGACTCCGT | | |
| | | CAAGGGCCGGTTCACCATCTCCAGGGAC | | |
| | | AACGCAAAGAAGACCCTCTACCTCCAGA | | |
| | | TGAACTCACTCAAGCCCGAGGACACCGC | | |
| | | TGTGTACTACTGCGCAGCCAGACGCATC | | |
| | | GATGCCGCAGACTTCGACTCCTGGGGCC | | |
| | | AAGGTACCCAAGTGACAGTGTCCAGCAT | | |
| | | CGAGGTGATGTACCCACCTCCCTACCTCG | | |
| | | ACAACGAGAAGAGCAACGGCACCATCAT | | |
| | | CCACGTGAAGGGGAAGCACCTGTGTCCC | | |
| | | TCTCCCCTTTTCCCAGGACCCTCCAAGCC | | |
| | | ATTCTGGGTCCTGGTCGTTGTCGGAGGCG | | |
| | | TGCTCGCTTGCTATTCCCTGCTCGTCACC | | |
| | | GTGGCCTTCATCATCTTCTGGGTGCGGTC | | |
| | | CAAGAGATCCCGGCTGCTGCACTCTGAT | | |
| | | TACATGAACATGACACCCAGGAGGCCAG | | |
| | | GGCCTACCAGGAAGCACTACCAGCCCTA | | |
| | | CGCTCCTCCACGCGACTTCGCAGCATACC | | |
| | | GGTCCCGCGTCAAGTTCTCCCGGTCCGCA | | |
| | | GATGCTCCAGCCTATCAGCAGGGCCAGA | | |
| | | ACCAGCTGTACAACGAACTCAATTTGGG | | |
| | | GAGGCGCGAGGAGTATGATGTGCTCGAT | | |
| | | AAGAGAAGGGGCCGGGATCCTGAGATG | | |
| | | GGAGGCAAGCCCAGACGGAAGAACCCTC | | |
| | | AGGAGGGCCTGTACAATGAGCTGCAGAA | | |
| | | GGACAAGATGGCCGAGGCCTACTCCGAG | | |
| | | ATCGGGATGAAGGGTGAAAGAAGGAGG | | |
| | | GGTAAGGGGCACGACGGGCTCTACCAAG | | |
| | | GCCTGAGCACCGCTACCAAGGACACCTA | | |
| | | CGATGCACTGCATATGCAAGCCCTGCCA | | |
| | | CCACGG | | |
| BCMA | 115 | ATGGCACTCCCGGTAACCGCCTTATTGCT | 131 | MALPVTALLLPLALLLHAARPDI |
| | | TCCCCTTGCCCTCTTGCTCCACGCAGCAC | | VLTQSPPSLAMSLGKRATISCRA |
| | | GCCCCGATATAGTCTTGACTCAATCCCCA | | SESVTILGSHLIHWYQQKPGQPP |
| | | CCCAGTTTGGCAATGTCATTAGGCAAAC | | TLLIQLASNVQTGVPARFSGSGS |
| | | GAGCAACAATTTCATGTAGGGCATCCGA | | RTDFTLTIDPVEEDDVAVYYCLQ |
| | | AAGTGTAACGATTTTGGGGAGTCATTTA | | SRTIPRTFGGGTKLEIKGSTSGSG |
| | | ATTCATTGGTACCAACAAAAGCCTGGAC | | KPGSGEGSTKGQIQLVQSGPELK |
| | | AACCCCCGACGCTCTTGATCCAATTAGC | | KPGETVKISCKASGYTFTDYSIN |
| | | ATCTAACGTCCAAACCGGAGTCCCCGCA | | WVKRAPGKGLKWMGWINTETR |
| | | CGATTCTCAGGATCCGGTTCCCGGACTG | | EPAYAYDFRGRFAFSLETSASTA |
| | | ATTTTACATTAACTATTGATCCGGTAGAG | | YLQINNLKYEDTATYFCALDYS |
| | | GAAGATGACGTCGCTGTCTATTATTGTCT | | YAMDYWGQGTSVTVSSAAATT |
| | | TCAAAGTAGGACGATTCCACGGACATTC | | TPAPRPPTPAPTIASQPLSLRPEA |
| | | GGTGGCGGAACTAAATTGGAGATTAAAG | | CRPAAGGAVHTRGLDFACDIYI |
| | | GTTCCACCTCTGGTAGTGGGAAACCCGG | | WAPLAGTCGVLLLSLVITLYCKR |
| | | GTCCGGTGAAGGGTCCACTAAAGGCCAA | | GRKKLLYIFKQPFMRPVQTTQEE |
| | | ATTCAACTCGTTCAATCCGGACCAGAAC | | DGCSCRFPEEEEGGCELRVKFSR |
| | | TGAAGAAGCCAGGAGAAACTGTCAAAAT | | SADAPAYQQGQNQLYNELNLGR |
| | | AAGCTGTAAAGCTTCCGGTTATACATTTA | | REEYDVLDKRRGRDPEMGGKPR |
| | | CAGATTATTCCATAAATTGGGTGAAAAG | | RKNPQEGLYNELQKDKMAEAYS |
| | | GGCGCCAGGAAAAGGGTTAAAGTGGATG | | EIGMKGERRRGKGHDGLYQGLS |
| | | GGTTGGATTAATACAGAGACTCGGGAAC | | TATKDTYDALHMQALPPR |
| | | CTGCATATGCTTATGATTTTAGGGGAAG | | |
| | | GTTTGCCTTTTCTCTGGAGACTTCCGCTT | | |
| | | CAACTGCTTATCTCCAAATTAATAATCTT | | |
| | | AAATATGAGGACACAGCAACATACTTCT | | |
| | | GTGCTTTGGACTATAGTTATGCTATGGAT | | |
| | | TACTGGGGACAAGGAACCAGTGTCACTG | | |
| | | TAAGTTCCGCTGCTGCGACGACCACTCCT | | |
| | | GCACCGCGACCACCCACTCCTGCCCCTA | | |
| | | CTATTGCTAGTCAACCACTTAGCTTGCGA | | |
| | | CCTGAGGCATGTCGGCCCGCGGCAGGTG | | |
| | | GCGCAGTCCACACCAGGGGTTTAGACTT | | |
| | | TGCTTGTGATATTTATATTTGGGCACCAC | | |
| | | TCGCCGGGACTTGCGGTGTTCTTCTCTTG | | |
| | | TCCCTTGTTATAACTCTTTATTGTAAGCG | | |
| | | CGGAAGGAAGAAATTGTTATATATTTTC | | |
| | | AAACAACCTTTTATGCGACCCGTACAAA | | |
| | | CAACTCAGGAAGAGGACGGGTGTTCTTG | | |
| | | TCGGTTTCCAGAAGAGGAAGAGGGTGGG | | |
| | | TGTGAACTCCGGGTCAAATTTAGTAGGT | | |
| | | CAGCAGATGCGCCGGCGTACCAACAAGG | | |
| | | CCAAAACCAACTGTATAATGAACTCAAT | | |
| | | CTCGGTAGGCGTGAGGAATATGATGTCC | | |
| | | TTGATAAAAGGCGCGGGAGAGATCCAGA | | |
| | | AATGGGCGGAAAACCACGGCGAAAGAA | | |
| | | TCCGCAGGAAGGGTTATATAACGAACTT | | |
| | | CAAAAGGATAAAATGGCTGAAGCTTATT | | |
| | | CCGAAATTGGCATGAAAGGAGAGCGACG | | |
| | | TAGGGGCAAAGGGCATGATGGCCTTTAC | | |
| | | CAAGGGCTCTCAACCGCTACAAAAGATA | | |
| | | CTTACGACGCTTTACATATGCAAGCACTT | | |
| | | CCACCCAGG | | |
| HER2 | 132 | ATGGCTCTGCCTGTGACAGCTCTGCTGCT | 134 | MALPVTALLLPLALLLHAARPDI |
| | | GCCTCTGGCTCTGCTTCTGCATGCCGCCA | | QMTQSPSSLSASVGDRVTITCRA |
| | | GACCTGACATCCAGATGACTCAGAGCCC | | SQDVNTAVAWYQQKPGKAPKL |
| | | CAGCAGCCTGTCTGCCTCTGTGGGAGAC | | LIYSASFLYSGVPSRFSGSRSGTD |
| | | AGAGTGACAATTACCTGCCGGGCCAGCC | | FTLTISSLQPEDFATYYCQQHYT |
| | | AGGATGTGAATACTGCTGTCGCCTGGTA | | TPPTFGQGTKVEIKRTGSTSGSG |
| | | TCAACAAAAGCCTGGCAAGGCCCCTAAG | | KPGSGEGSEVQLVESGGGLVQP |
| | | CTCCTGATCTACAGCGCCAGCTTTCTGTA | | GGSLRLSCAASGFNIKDTYIHWV |
| | | CAGCGGCGTGCCCAGCAGATTCTCCGGA | | RQAPGKGLEWVARIYPTNGYTR |
| | | AGCAGAAGCGGCACAGATTTCACACTGA | | YADSVKGRFTISADTSKNTAYLQ |
| | | CCATAAGCAGCCTGCAGCCAGAGGATTT | | MNSLRAEDTAVYYCSRWGGDG |
| | | CGCCACCTACTATTGCCAGCAGCACTAC | | FYAMDVWGQGTLVTVSSIEVMY |
| | | ACCACACCTCCAACCTTTGGCCAGGGCA | | PPPYLDNEKSNGTIIHVKGKHLC |
| | | CCAAGGTCGAGATTAAGAGAACAGGCAG | | PSPLFPGPSKPFWVLVVVGGVLA |
| | | CACATCTGGCTCTGGCAAACCTGGATCT | | CYSLLVTVAFIIFWVRSKRSRLL |
| | | GGCGAGGGCTCTGAAGTCCAGCTGGTGG | | HSDYMNMTPRRPGPTRKHYQPY |
| | | AATCTGGCGGAGGACTGGTTCAACCTGG | | APPRDFAAYRSRVKFSRSADAPA |
| | | CGGCTCTCTGAGACTGTCTTGTGCCGCCT | | YQQGQNQLYNELNLGRREEYDV |
| | | CCGGCTTCAACATCAAGGACACCTACAT | | LDKRRGRDPEMGGKPRRKNPQE |
| | | CCACTGGGTCCGACAAGCCCCAGGCAAA | | GLYNELQKDKMAEAYSEIGMKG |
| | | GGACTTGAGTGGGTCGCCAGGATCTACC | | ERRRGKGHDGLYQGLSTATKDT |
| | | CCACCAACGGCTACACCAGATACGCCGA | | YDALHMQALPPR |
| | | CTCTGTGAAGGGCAGATTCACCATCTCTG | | |
| | | CCGACACCAGCAAGAATACCGCCTACCT | | |
| | | GCAGATGAACTCCCTGAGAGCCGAAGAT | | |
| | | ACCGCTGTGTATTACTGTTCCAGATGGGG | | |
| | | AGGCGACGGCTTCTACGCCATGGATGTT | | |
| | | TGGGGCCAAGGCACCCTCGTGACCGTTT | | |
| | | CTTCTATCGAAGTGATGTACCCTCCACCT | | |
| | | TACCTGGACAACGAGAAGTCCAACGGCA | | |
| | | CCATCATCCACGTGAAGGGCAAGCACCT | | |
| | | GTGTCCTTCTCCACTGTTCCCCGGACCTA | | |
| | | GCAAGCCTTTCTGGGTGCTCGTTGTTGTT | | |
| | | GGCGGCGTGCTGGCCTGTTACTCTCTGCT | | |
| | | GGTTACCGTGGCCTTCATCATCTTTTGGG | | |
| | | TCCGAAGCAAGCGGAGCCGGCTGCTGCA | | |
| | | CTCCGACTACATGAACATGACCCCTAGA | | |
| | | CGGCCCGGACCAACCAGAAAGCACTACC | | |
| | | AGCCTTACGCTCCTCCTAGAGACTTCGCC | | |
| | | GCCTACCGGTCCAGAGTGAAGTTCAGCA | | |
| | | GATCCGCCGATGCTCCCGCCTATCAGCA | | |
| | | GGGCCAAAACCAGCTGTACAACGAGCTG | | |
| | | AACCTGGGGAGAAGAGAAGAGTACGAC | | |
| | | GTGCTGGACAAGCGGAGAGGCAGAGATC | | |
| | | CTGAAATGGGCGGCAAGCCCAGACGGAA | | |
| | | GAATCCTCAAGAGGGCCTGTATAATGAG | | |
| | | CTGCAGAAAGACAAGATGGCCGAGGCCT | | |
| | | ACAGCGAGATCGGAATGAAGGGCGAGC | | |
| | | GCAGAAGAGGCAAGGGACACGATGGAC | | |
| | | TGTACCAGGGCCTGAGCACCGCCACCAA | | |
| | | GGATACCTATGATGCCCTGCACATGCAG | | |
| | | GCCCTGCCTCCAAGA | | |
| HER2 | 133 | ATGGCTCTGCCTGTGACAGCTCTGCTGCT | 135 | MALPVTALLLPLALLLHAARPDI |
| | | GCCTCTGGCTCTGCTTCTGCATGCCGCCA | | QMTQSPSSLSASVGDRVTITCRA |
| | | GACCTGACATCCAGATGACTCAGAGCCC | | SQDVNTAVAWYQQKPGKAPKL |
| | | CAGCAGCCTGTCTGCCTCTGTGGGAGAC | | LIYSASFLYSGVPSRFSGSRSGTD |
| | | AGAGTGACAATTACCTGCCGGGCCAGCC | | FTLTISSLQPEDFATYYCQQHYT |
| | | AGGATGTGAATACTGCTGTCGCCTGGTA | | TPPTFGQGTKVEIKRTGSTSGSG |
| | | TCAACAAAAGCCTGGCAAGGCCCCTAAG | | KPGSGEGSEVQLVESGGGLVQP |
| | | CTCCTGATCTACAGCGCCAGCTTTCTGTA | | GGSLRLSCAASGFNIKDTYIHWV |
| | | CAGCGGCGTGCCCAGCAGATTCTCCGGA | | RQAPGKGLEWVARIYPINGYTR |
| | | AGCAGAAGCGGCACAGATTTCACACTGA | | YADSVKGRFTISADTSKNTAYLQ |
| | | CCATAAGCAGCCTGCAGCCAGAGGATTT | | MNSLRAEDTAVYYCSRWGGDG |
| | | CGCCACCTACTATTGCCAGCAGCACTAC | | FYAMDVWGQGTLVTVSSTTTPA |
| | | ACCACACCTCCAACCTTTGGCCAGGGCA | | PRPPTPAPTIASQPLSLRPEACRP |
| | | CCAAGGTCGAGATTAAGAGAACAGGCAG | | AAGGAVHTRGLDFACDIYIWAP |
| | | CACATCTGGCTCTGGCAAACCTGGATCT | | LAGTCGVLLLSLVITLYCKRGRK |
| | | GGCGAGGGCTCTGAAGTCCAGCTGGTGG | | KLLYIFKQPFMRPVQTTQEEDGC |
| | | AATCTGGCGGAGGACTGGTTCAACCTGG | | SCRFPEEEEGGCELRVKFSRSAD |
| | | CGGCTCTCTGAGACTGTCTTGTGCCGCCT | | APAYQQGQNQLYNELNLGRREE |
| | | CCGGCTTCAACATCAAGGACACCTACAT | | YDVLDKRRGRDPEMGGKPRRK |
| | | CCACTGGGTCCGACAAGCCCCAGGCAAA | | NPQEGLYNELQKDKMAEAYSEI |
| | | GGACTTGAGTGGGTCGCCAGGATCTACC | | GMKGERRRGKGHDGLYQGLST |
| | | CCACCAACGGCTACACCAGATACGCCGA | | ATKDTYDALHMQALPPR |
| | | CTCTGTGAAGGGCAGATTCACCATCTCTG | | |
| | | CCGACACCAGCAAGAATACCGCCTACCT | | |
| | | GCAGATGAACTCCCTGAGAGCCGAAGAT | | |
| | | ACCGCTGTGTATTACTGTTCCAGATGGGG | | |
| | | AGGCGACGGCTTCTACGCCATGGATGTT | | |
| | | TGGGGCCAAGGCACCCTCGTGACCGTTT | | |
| | | CTTCTACCACCACACCAGCTCCTCGGCCT | | |
| | | CCAACTCCTGCTCCTACAATTGCCAGCCA | | |
| | | GCCTCTGTCTCTGAGGCCCGAAGCTTGTA | | |
| | | GACCTGCTGCTGGCGGAGCCGTGCATAC | | |
| | | AAGAGGACTGGATTTCGCCTGCGACATC | | |
| | | TACATCTGGGCTCCTCTGGCCGGAACAT | | |
| | | GTGGCGTTCTGCTGCTGAGCCTGGTCATC | | |
| | | ACCCTGTACTGTAAGCGGGGCAGAAAGA | | |
| | | AGCTGCTGTACATCTTCAAGCAGCCCTTC | | |
| | | ATGCGGCCCGTGCAGACCACACAAGAGG | | |
| | | AAGATGGCTGCTCCTGCAGATTCCCCGA | | |
| | | GGAAGAAGAAGGCGGCTGCGAGCTGAG | | |
| | | AGTGAAGTTCAGCAGATCCGCCGATGCT | | |
| | | CCCGCCTATCAGCAGGGCCAAAACCAGC | | |
| | | TGTACAACGAGCTGAACCTGGGGAGAAG | | |
| | | AGAAGAGTACGACGTGCTGGACAAGCGG | | |
| | | AGAGGCAGAGATCCTGAAATGGGCGGCA | | |
| | | AGCCCAGACGGAAGAATCCTCAAGAGGG | | |
| | | CCTGTATAATGAGCTGCAGAAAGACAAG | | |
| | | ATGGCCGAGGCCTACAGCGAGATCGGAA | | |
| | | TGAAGGGCGAGCGCAGAAGAGGCAAGG | | |
| | | GACACGATGGACTGTACCAGGGCCTGAG | | |
| | | CACCGCCACCAAGGATACCTATGATGCC | | |
| | | CTGCACATGCAGGCCCTGCCTCCAAGA | | |

### 3. Hinge/Spacer Domain

In some embodiments, a CAR of the instant disclosure comprises a hinge or spacer domain. In some embodiments, the hinge/spacer domain may comprise a truncated hinge/spacer domain (THD), wherein the THD domain is a truncated version of a complete hinge/spacer domain (“CHD”). In some embodiments, an hinge or spacer domain is from or derived from (e.g., comprises all or a fragment of) ErbB2, glycophorin A (GpA), CD2, CD3 delta, CD3 epsilon, CD3 gamma, CD4, CD7, CD8a, CD8 [T CD1 1a (IT GAL), CD1 1b (IT GAM), CD1 1c (ITGAX), CD1 1d (IT GAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3 DPI), CD158D (KIRDL4), CD158F1 (KIR2DL5A), CD158F2 (KIR2DL5B), CD158K (KIR3DL2), CD160 (BY55), CD162 (SELPLG), CD226 (DNAMI), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRT AM), CD357 (TNFRSF18), inducible T cell co-stimulator (ICOS), LFA-1 (CD1 1a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof. A hinge or spacer domain may be derived either from a natural or from a synthetic source.

In some embodiments, a hinge or spacer domain is positioned between an antigen binding molecule (e.g., an scFv) and a transmembrane domain. In this orientation, the hinge/spacer domain provides distance between the antigen binding molecule and the surface of a cell membrane on which the CAR is expressed. In some embodiments, a hinge or spacer domain is from or derived from an immunoglobulin. In some embodiments, a hinge or spacer domain is selected from the hinge/spacer regions of IgG1, IgG2, IgG3, IgG4, IgA, IgD, IgE, IgM, or a fragment thereof. In some embodiments, a hinge or spacer domain comprises, is from, or is derived from the hinge/spacer region of CD8 alpha. In some embodiments, a hinge or spacer domain comprises, is from, or is derived from the hinge/spacer region of CD28. In some embodiments, a hinge or spacer domain comprises a fragment of the hinge/spacer region of CD8 alpha or a fragment of the hinge/spacer region of CD28, wherein the fragment is anything less than the whole hinge/spacer region. In some embodiments, the fragment of the CD8 alpha hinge/spacer region or the fragment of the CD28 hinge/spacer region comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of the CD8 alpha hinge/spacer region, or of the CD28 hinge/spacer region.

### 4. Transmembrane Domain

The CAR of the present disclosure may further comprise a transmembrane domain and/or an intracellular signaling domain. The transmembrane domain may be designed to be fused to the extracellular domain of the CAR. It may similarly be fused to the intracellular domain of the CAR. In some embodiments, the transmembrane domain that naturally is associated with one of the domains in a CAR is used. In some instances, the transmembrane domain may be selected or modified (e.g., by an amino acid substitution) to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein.

Transmembrane regions may be derived from (i.e., comprise) a receptor tyrosine kinase (e.g., ErbB2), glycophorin A (GpA), 4-1BB/CD137, activating NK cell receptors, an immunoglobulin protein, B7-H3, BAFFR, BFAME (SEAMF8), BTEA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD1 1a, CD1 1b, CD1 1c, CD1 1d, CDS, CEACAM1, CRT AM, cytokine receptor, DAP-10, DNAMI (CD226), Fc gamma receptor, GADS, GITR, HVEM (EIGHTR), IA4, ICAM-1, ICAM-1, Ig alpha (CD79a), IE-2R beta, IE-2R gamma, IE-7R alpha, inducible T cell costimulator (ICOS), integrins, ITGA4, ITGA4, ITGA6, IT GAD, ITGAE, ITGAE, IT GAM, ITGAX, ITGB2, ITGB7, ITGB1, KIRDS2, EAT, LFA-1, LFA-1, a ligand that specifically binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

In some embodiments, suitable intracellular signaling domain include, but are not limited to, activating Macrophage/Myeloid cell receptors CSFR1, MYD88, CD14, TIE2, TLR4, CR3, CD64, TREM2, DAP10, DAP12, CD169, DECTIN1, CD206, CD47, CD163, CD36, MARCO, TIM4, MERTK, F4/80, CD91, C1QR, LOX-1, CD68, SRA, BAI-1, ABCA7, CD36, CD31, Lactoferrin, or a fragment, truncation, or combination thereof.

In some embodiments, a receptor tyrosine kinase may be derived from (e.g., comprise) Insulin receptor (InsR), Insulin-like growth factor I receptor (IGFIR), Insulin receptor-related receptor (IRR), platelet derived growth factor receptor alpha (PDGFRa), platelet derived growth factor receptor beta (PDGFRfi). KIT proto-oncogene receptor tyrosine kinase (Kit), colony stimulating factor 1 receptor (CSFR), fis related tyrosine kinase 3 (FLT3), fms related tyrosine kinase 1 (VEGFR-1), kinase insert domain receptor (VEGFR-2), fms related tyrosine kinase 4 (VEGFR-3), fibroblast growth factor receptor 1 (FGFR1), fibroblast growth factor receptor 2 (FGFR2), fibroblast growth factor receptor 3 (FGFR3), fibroblast growth factor receptor 4 (FGFR4), protein tyrosine kinase 7 (CCK4), neurotrophic receptor tyrosine kinase 1 (trkA), neurotrophic receptor tyrosine kinase 2 (trkB), neurotrophic receptor tyrosine kinase 3 (trkC), receptor tyrosine kinase like orphan receptor 1 (ROR1), receptor tyrosine kinase like orphan receptor 2 (ROR2), muscle associated receptor tyrosine kinase (MuSK), MET proto-oncogene, receptor tyrosine kinase (MET), macrophage stimulating 1 receptor (Ron), AXL receptor tyrosine kinase (Axl), TYR03 protein tyrosine kinase (Tyro3), MER proto-oncogene, tyrosine kinase (Mer), tyrosine kinase with immunoglobulin like and EGF like domains 1 (TIE1), TEK receptor tyrosine kinase (TIE2), EPH receptor A1 (EphAl), EPH receptor A2 (EphA2), (EPH receptor A3) EphA3, EPH receptor A4 (EphA4), EPH receptor A5 (EphA5), EPH receptor A6 (EphA6), EPH receptor A7 (EphA7), EPH receptor A8 (EphA8), EPH receptor A10 (EphAIO), EPH receptor B1 (EphB1), EPH receptor B2 (EphB2), EPH receptor B3 (EphB3), EPH receptor B4 (EphB4), EPH receptor B6 (EphB6), ret proto oncogene (Ret), receptor-like tyrosine kinase (RYK), discoidin domain receptor tyrosine kinase 1 (DDR1), discoidin domain receptor tyrosine kinase 2 (DDR2), c-ros oncogene 1, receptor tyrosine kinase (ROS), apoptosis associated tyrosine kinase (Lmrl), lemur tyrosine kinase 2 (Lmr2), lemur tyrosine kinase 3 (Lmr3), leukocyte receptor tyrosine kinase (LTK), ALK receptor tyrosine kinase (ALK), or serine/threonine/tyrosine kinase 1 (STYK1).

### 5. Costimulatory Domain

In certain embodiments, the CAR comprises a costimulatory domain. In some embodiments, the costimulatory domain comprises 4-1BB (CD137), CD28, or both, and/or an intracellular T cell signaling domain. In a preferred embodiment, the costimulatory domain is human CD28, human 4-1BB, or both, and the intracellular T cell signaling domain is human CD3 zeta (ζ). 4-1BB, CD28, CD3 zeta may comprise less than the whole 4-1BB, CD28 or CD3 zeta, respectively. Chimeric antigen receptors may incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Amur. Rev. Pharmacol. Toxicol. 56:59-83 (2016).

### 6. Intracellular Signaling Domain

In certain embodiments, the CAR comprises an intracellular (signaling) domain.

In some embodiments, suitable intracellular signaling domains comprise, but are not limited, to 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD 19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD1 1a, CD1 1b, CD1 1c, CD1 1d, CDS, CEACAM1, CRT AM, cytokine receptor, DAP-10, DNAMI (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, LFA-1, ligand that specifically binds with CD83, LIGHT, LTBR, Ly9 (CD229), Ly108, lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

CD3 is an element of the T cell receptor on native T cells, and has been shown to be an important intracellular activating element in CARs. In some embodiments, the CD3 is CD3 zeta.

### E. Transfer Vehicles

In one aspect, provided herein is a pharmaceutical composition comprising one or more circular RNA constructs comprising an Internal Ribosome Entry Site (IRES) and an expression sequence encoding a binding molecule and a transfer vehicle. In certain embodiments, the pharmaceutical composition comprises at least one circular RNA construct comprising an IRES and an expression sequence encoding a chimeric antigen receptor (CAR); and a transfer vehicle.

In certain embodiments, the transfer vehicle comprises a lipid. In certain embodiments, the transfer vehicle comprises an ionizable lipid. In certain embodiments, the transfer vehicle comprises an ionizable lipid in combination with other lipids, e.g., a structural lipid, and/or a PEG-modified lipid. In certain embodiments, the transfer vehicle is a lipid nanoparticle (LNP). In certain embodiments the transfer vehicle is capable of delivering the circular RNA construct to a human immune cell present in a human subject, such that the expression sequence encoding a binding molecule (e.g., CAR) is translated in the human immune cell and expressed on the surface of the human immune cell.

In certain embodiments, the transfer vehicles are prepared to encapsulate one or more materials or therapeutic agents (e.g., circular RNA). The process of incorporating a desired therapeutic agent (e.g., circular RNA) into a transfer vehicle is referred to herein as or “loading” or “encapsulating” (Lasic, et al., FEBS Lett., 312:255-258, 1992). The transfer vehicle-loaded or -encapsulated materials (e.g., circular RNA) may be completely or partially located in the interior space of the transfer vehicle, within a bilayer membrane of the transfer vehicle, or associated with the exterior surface of the transfer vehicle.

In some embodiments, a transfer vehicle encapsulates circular RNA. In some embodiments, the transfer vehicle encapsulates at least one circular RNA construct and comprises an ionizable lipid. In some embodiments, the transfer vehicle encapsulates at least one circular RNA construct and comprises an ionizable lipid and an additional lipid selected from a structural lipid, a helper lipid, and a PEG-modified lipid. In some embodiments, the transfer vehicle encapsulates at least one circular RNA construct and comprises an ionizable lipid, a structural lipid, a helper lipid, and/or a PEG-modified lipid. In some embodiments, a transfer vehicle encapsulates at least one circular RNA construct and comprises an ionizable lipid, a structural lipid, a PEG-modified lipid, and a helper lipid. In some embodiments, the transfer vehicle is a lipid nanoparticle.

Without wishing to be bound by theory, it is thought that transfer vehicles described herein shield encapsulated circular RNA from degradation and provide for effective delivery of circular RNA to target cells in vivo and in vitro.

In certain embodiments, the transfer vehicles are formulated based in part upon their ability to facilitate the transfection (e.g., of a circular RNA) of a target cell. In another embodiment, the transfer vehicles may be selected and/or prepared to optimize delivery of circular RNA to a target cell, tissue or organ. For example, if the target cell is a hepatocyte, the properties of the compositions (e.g., size, charge and/or pH) may be optimized to effectively deliver such composition (e.g., lipid nanoparticles) to the target cell or organ, reduce immune clearance and/or promote retention in the target cell or organ. Alternatively, if the target tissue is the central nervous system, the selection and preparation of the transfer vehicle must consider penetration of, and retention within. the blood brain barrier and/or the use of alternate means of directly delivering such compositions to such target tissue (e.g., via intracerebrovascular administration). In certain embodiments, the transfer vehicles may be combined with agents that facilitate the transfer of encapsulated materials across the blood brain barrier (e.g., agents which disrupt or improve the permeability of the blood brain barrier and thereby enhance the transfer of circular RNA to the target cells). While the transfer vehicles described herein can facilitate introduction of circular RNA into target cells, the addition of polycations (e.g., poly L-lysine and protamine) as a copolymer to one or more of the lipid nanoparticles that comprise the pharmaceutical compositions can in some instances markedly enhance the transfection efficiency of several types of transfer vehicles by 2-28 fold in a number of cell lines both in vitro and in vivo (See, N. J. Caplen, et al., Gene Ther. 1995; 2:603; S. Li, et al., Gene Ther. 1997; 4, 891.).

Transfer vehicles described herein can allow the encapsulated polynucleotide to reach the target cell or may preferentially allow the encapsulated polynucleotide to reach the target cells or organs on a discriminatory basis. Alternatively, the transfer vehicles may limit the delivery of encapsulated polynucleotides to other non-targeted cells or organs where the presence of the encapsulated polynucleotides may be undesirable or of limited utility.

Loading or encapsulating a polynucleotide, e.g., circular RNA, into a transfer vehicle may serve to protect the polynucleotide from an environment (e.g., serum) which may contain enzymes or chemicals that degrade such polynucleotides and/or systems or receptors that cause the rapid excretion of such polynucleotides. Accordingly, in some embodiments, the compositions described herein are capable of enhancing the stability of the encapsulated polynucleotide(s), particularly with respect to the environments into which such polynucleotides will be exposed.

In certain embodiments, the transfer vehicles described herein are prepared by combining multiple lipid components (e.g., one or more of the compounds disclosed herein) with one or more polymer components.

A lipid nanoparticle may be comprised of additional lipid combinations in various ratios. Example 1 at Tables 4a and 4b describes exemplary lipid vehicle formulations comprising different molar ratios. The selection of ionizable lipids, helper lipids, structural lipids, and/or PEG-modified lipids that make up the lipid nanoparticles, as well as the relative molar ratio of such lipids to each other, is based upon the characteristics of the selected lipid(s), the nature of the intended target cells or tissues and the characteristics of the materials or polynucleotides to be delivered by the lipid nanoparticle. Additional considerations include, for example, the saturation of the alkyl chain, as well as the size, charge, pH, pKa, fusogenicity and toxicity of the selected lipid(s).

### 1. Lipid Nanoparticles (“LNP”)

In one embodiment, the circular RNA may be formulated in or encapsulated into a transfer or delivery vehicle where the vehicle is a lipid nanoparticle, which may be capable of delivering the one or more circular RNA constructs to one or more target cells.

The formation of a lipid nanoparticle (LNP) described herein may be accomplished by any methods known in the art. See, e.g., U.S. Pat. Pub. No. US2012/0178702 A1, which is incorporated herein by reference in its entirety. Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578 (the contents of each of which are incorporated herein by reference in their entirety). Lipid nanoparticles, formulations, and methods of preparation are described in, e.g., International Pat. Pub. No. WO 2011/127255 or WO 2008/103276, U.S. Pat. Pub. No. US2005/0222064 A1, U.S. Pat. Pub. No. US2013/0156845 A1, International Pat. Pub. No. WO2013/093648 A2, WO2012/024526 A2, U.S. Pat. Pub. No. US2013/0164400 A1, and U.S. Pat. No. 8,492,359, all of which are incorporated herein by reference in their entirety.

A lipid nanoparticle may be comprised of lipid combinations in various ratios. The selection of ionizable lipids, helper lipids, structural lipids, and/or PEG-modified lipids which comprise the lipid nanoparticles, as well as the relative molar ratio of such lipids to each other, is based upon the characteristics of the selected lipid(s), the nature of the intended target cells or tissues and the characteristics of the materials or polynucleotides to be delivered by the lipid nanoparticle. Additional considerations include, for example, the saturation of the alkyl chain, as well as the size, charge, pH, pKa, fusogenicity and toxicity of the selected lipid(s). Example 1 at Tables 4a and 4b describes exemplary lipid vehicle formulations comprising different molar ratios. Additional lipids are described in WO2022261490, WO2023056033, and WO2023081526, which are each incorporated herein by reference in their entireties.

In some embodiments, the lipid nanoparticle comprises one or more cationic lipids, ionizable lipids, or poly β-amino esters. In some embodiments, the nanoparticle comprises one or more non-cationic lipids. In some embodiments, the lipid nanoparticle comprises one or more PEG-modified lipids, polyglutamic acid lipids, or hyaluronic acid lipids. In some embodiments, the lipid nanoparticle comprises cholesterol. In some embodiments, the lipid nanoparticle comprises arachidonic acid, leukotriene, or oleic acid. In some embodiments, the lipid nanoparticle comprises a targeting moiety, wherein the targeting moiety mediates receptor-mediated endocytosis selectively into cells of a selected cell population in the absence of cell selection or purification. In some embodiments, the lipid nanoparticle comprises more than one circular RNA construct.

Examples of further suitable lipids include the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine.

A lipid nanoparticle composition may optionally comprise one or more coatings. For example, a nanoparticle composition may be formulated in a capsule, film, or tablet having a coating. A capsule, film, or tablet including a composition described herein may have any useful size, tensile strength, hardness, or density.

In one embodiment, the lipid nanoparticles may have a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm. In one embodiment, the lipid nanoparticles may have a diameter from about 10 to 500 nm. In one embodiment, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm. Each possibility represents a separate embodiment.

In some embodiments, a nanoparticle (e.g., a lipid nanoparticle) has a mean diameter of 10-500 nm, 20-400 nm, 30-300 nm, or 40-200 nm. In some embodiments, a nanoparticle (e.g., a lipid nanoparticle) has a mean diameter of 50-150 nm, 50-200 nm, 80-100 nm, or 80-200 nm.

In some embodiments, the lipid nanoparticles described herein can have a diameter from below 0.1 μm to up to 1 mm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, less than 975 μm.

In another embodiment, LNPs may have a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm. Each possibility represents a separate embodiment.

A nanoparticle composition may be relatively homogenous. A polydispersity index may be used to indicate the homogeneity of a nanoparticle composition, e.g., the particle size distribution of the lipid nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.1 1, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20. Each possibility represents a separate embodiment.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −20 mV to about +20 mV, from about −20 mV to about +15 mV, from about −20 mV to about +10 mV, from about −20 mV to about +5 mV, from about −20 m V to about 0 mV, from about −20 mV to about −5 mV, from about −20 mV to about −10 mV, from about −20 mV to about −15 mV from about −20 mV to about +20 m V, from about −20 m V to about +15 mV, from about −20 m V to about +10 mV, from about −20 mV to about +5 mV, from about −20 m V to about 0 mV, from about 0 m V to about +20 mV, from about 0 m V to about +15 mV, from about 0 m V to about +10 mV, from about 0 mV to about +5 mV, from about +5 mV to about +20 m V, from about +5 m V to about +15 m V, or from about +5 mV to about +10 mV. Each possibility represents a separate embodiment.

The efficiency of encapsulation of a therapeutic agent describes the amount of therapeutic agent that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic agent in a solution containing the lipid nanoparticle composition before and after breaking up the lipid nanoparticle composition with one or more organic solvents or detergents. Fluorescence may be used to measure the amount of free therapeutic agent (e.g., nucleic acids) in a solution. For the lipid nanoparticle compositions described herein, the encapsulation efficiency of a therapeutic agent may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

In some embodiments, the lipid nanoparticle has a polydiversity value of less than 0.4. In some embodiments, the lipid nanoparticle has a net neutral charge at a neutral pH. In some embodiments, the lipid nanoparticle has a mean diameter of 50-200 nm.

The properties of a lipid nanoparticle formulation may be influenced by factors including, but not limited to, the selection of the cationic lipid component, the degree of cationic lipid saturation, the selection of the non-cationic lipid component, the degree of noncationic lipid saturation, the selection of the structural lipid component, the nature of the PEGylation, ratio of all components and biophysical parameters such as size. As described herein, the purity of a PEG lipid component is also important to an LNP's properties and performance.

### a) Ionizable Lipids

In certain embodiments, the transfer vehicle comprises an ionizable lipid. Ionizable lipids may be used as a component of the transfer vehicle to facilitate or enhance the delivery and release of circular RNA to one or more target cells (e.g., by permeating or fusing with the lipid membranes of such target cells). In certain embodiments, an ionizable lipid comprises one or more cleavable functional groups (e.g., a disulfide) that allow, for example, a hydrophilic functional head-group to dissociate from a lipophilic functional tail-group of the compound (e.g., upon exposure to oxidative, reducing or acidic conditions), thereby facilitating a phase transition in the lipid bilayer of the one or more target cells.

In some embodiments, an ionizable lipid is as described in international patent application PCT/US2020/038678. In some embodiments, an ionizable lipid is a lipid as represented by formula 1 of or as listed in Tables 1 or 2 of U.S. Pat. No. 9,708,628, the content of which is herein incorporated by reference in its entirety. In some embodiments, an ionizable lipid is as described in pages 7-13 of U.S. Pat. No. 9,765,022 or as represented by formula 1 of U.S. Pat. No. 9,765,022, the content of which is herein incorporated by reference in its entirety. In some embodiments, an ionizable lipid is described in pages 12-24 of International Patent Application No. PCT/US2019/016362 or as represented by formula 1 of International Patent Application PCT/US2019/016362, the contents of which are herein incorporated by reference in their entirety. In some embodiments, a lipid or transfer vehicle is a lipid as described in International Patent Application Nos. PCT/US2010/061058, PCT/US2018/058555, PCT/US2018/053569, PCT/US2017/028981, PCT/US2019/025246, PCT/US2019/015913, PCT/US2019/016362, PCT/US2019/016362, US Application Publication Nos. US2019/0314524, US2019/0321489, US2019/0314284, and US2019/0091164, the contents of which are herein incorporated by reference in their entireties. Suitable cationic lipids for use in the compositions and methods herein include those described in international patent publication WO 2010/053572 and/or U.S. patent application Ser. No. 15/809,680, e.g., C12-200. In certain embodiments, the compositions and methods herein employ a lipid nanoparticles comprising an ionizable cationic lipid described in U.S. provisional patent application 61/617,468, filed Mar. 29, 2012 (incorporated herein by reference), such as, e.g, (15Z,18Z)—N,N-dimethyl-6-(9Z,12Z)-octadeca-9,12-dien-1-yl)tetracosa-15,18-dien-1-amine (HGT5000), (15Z,18Z)—N,N-dimethyl-6-((9Z,12Z)-octadeca-9,12-dien-1-yl)tetracosa-4,15,18-trien-1-amine (HGT5001), and (15Z,18Z)—N,N-dimethyl-6-((9Z,12Z)-octadeca-9,12-dien-1-yl)tetracosa-5,15,18-trien-1-amine (HGT5002).

In some embodiments, the cationic lipid N-[1-(2,3-dioleyloxy) propyl]-N,N,N-trimethylammonium chloride or “DOTMA” is used. (Felgner et al. (Proc. Nat'l Acad. Sci. 84, 7413 (1987); U.S. Pat. No. 4,897,355). DOTMA can be formulated alone or can be combined with the neutral lipid, dioleoylphosphatidyl-ethanolamine or “DOPE” or other cationic or non-cationic lipids into a transfer vehicle or a lipid nanoparticle, and such liposomes can be used to enhance the delivery of nucleic acids into target cells. Other suitable cationic lipids include, for example, 5-carboxyspermylglycinedioctadecylamide or “DOGS,” 2,3-dioleyloxy-N-[2 (spermine-carboxamido)ethyl]-N,N-dimethyl-1-propanaminium or “DOSPA” (Behr et al. Proc. Nat.′I Acad. Sci. 86, 6982 (1989); U.S. Pat. Nos. 5,171,678; 5,334,761), 1,2-Dioleoyl-3-Dimethylammonium-Propane or “DODAP,” 1,2-Dioleoyl-3-Trimethylammonium-Propane or “DOTAP.” Contemplated cationic lipids also include 1,2-distearyloxy-N,N-dimethyl-3-aminopropane or “DSDMA”, 1,2-dioleyloxy-N,N-dimethyl-3-aminopropane or “DODMA,” 1,2-dilinoleyloxy-N,N-dimethyl-3-aminopropane or “DLinDMA,” 1,2-dilinolenyloxy-N,N-dimethyl-3-aminopropane or “DLenDMA,” N-dioleyl-N,N-dimethylammonium chloride or “DODAC,” N,N-distearyl-N,N-dimethylammonium bromide or “DDAB,” N-(1,2-dimyristyloxyprop-3-yl)-N,N-dimethyl-N-hydroxyethyl ammonium bromide or 3-dimethylamino-2-(cholest-5-en-3-beta-oxybutan-4-oxy)-1-(cis,cis-9,12-‘DMRIE,” octadecadienoxy) propane or “CLinDMA,” 2-[5’-(cholest-5-en-3-beta-oxy)-3′-oxapentoxy)-3-dimethy 1-1-(cis, cis-9′, 1-2′-octadecadienoxy) propane or “CpLinDMA,” N,N-dimethyl-3,4-dioleyloxybenzylamine or “DMOBA,” 1,2-N,N′-dioleylcarbamyl-3-dimethylaminopropane or “DOcarbDAP,” 2,3-Dilinoleoyloxy-N,N-dimethylpropylamine or “DLinDAP,” 1,2-N,N′-Dilinoleylcarbamyl-3-dimethylaminopropane or “DLincarbDAP,” 1,2-Dilinoleoylcarbamyl-3-dimethylaminopropane or “DLinCDAP,” 2,2-dilinoleyl-4-dimethylaminomethyl-[1,3]-dioxolane or “DLin-K-DMA,” 2,2-dilinoleyl-4-dimethylaminoethyl-[1,3]-dioxolane or “DLin-K-XTC2-DMA,” and 2-(2,2-di((9Z,12Z)-octadeca-9,12-dien-1-yl)-1,3-dioxolan-4-yl)-N,N-dimethylethanamine (DLin-KC2-DMA)) (See, WO 2010/042877; Semple et al., Nature Biotech. 28:172-176 (2010)), or mixtures thereof. (Heyes, J., et al., J Controlled Release 107:276-287 (2005); Morrissey, D V., et al., Nat. Biotechnol. 23 (8): 1003-1007 (2005); PCT Publication WO2005/121348A1).

The use of cholesterol-based cationic lipids is also contemplated herein. Such cholesterol-based cationic lipids can be used, either alone or in combination with other cationic or non-cationic lipids. Suitable cholesterol-based cationic lipids include, for example, GL67, DC-Chol (N,N-dimethyl-N-ethylcarboxamidocholesterol), 1,4-bis(3-N-oleylamino-propyl) piperazine (Gao, et al. Biochem. Biophys. Res. Comm. 179, 280 (1991); Wolf et al. BioTechniques 23, 139 (1997); U.S. Pat. No. 5,744,335), or ICE.

In some embodiments, the one or more of the cationic or ionizable lipids provide increased activity of the nucleic acid and improved tolerability of the compositions in vivo.

In some embodiments, the one or more of the cationic or ionizable lipids are represented by Formula (I):

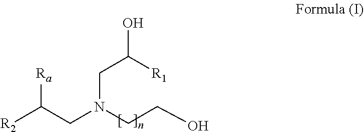

wherein:
   n is an integer between 1 and 4;
   Rₐ is hydrogen or hydroxyl; and
   R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl.

In some embodiments, Ra is hydrogen. In some embodiments, Ra is hydroxyl.

In some embodiments, the ionizable lipid is represented by Formula (Ia-1), Formula (Ia-2), or Formula (Ia-3):

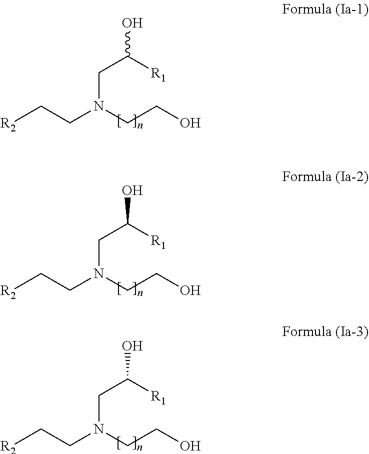

In some embodiments, the ionizable lipid is represented by Formula (Ib-1), Formula (Ib-2), or Formula (Ib-3):

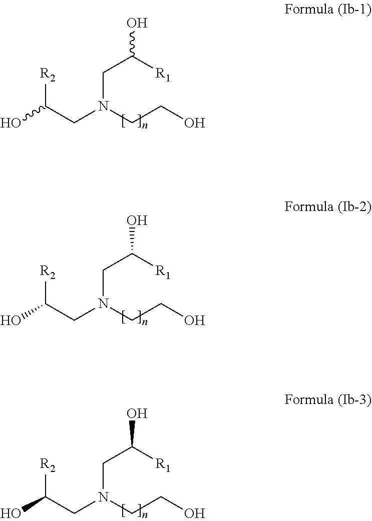

In some embodiments, the ionizable lipid is represented by Formula (Ib-4), Formula (Ib-5), Formula (Ib-6), Formula (Ib-7), Formula (Ib-8), or Formula (Ib-9):

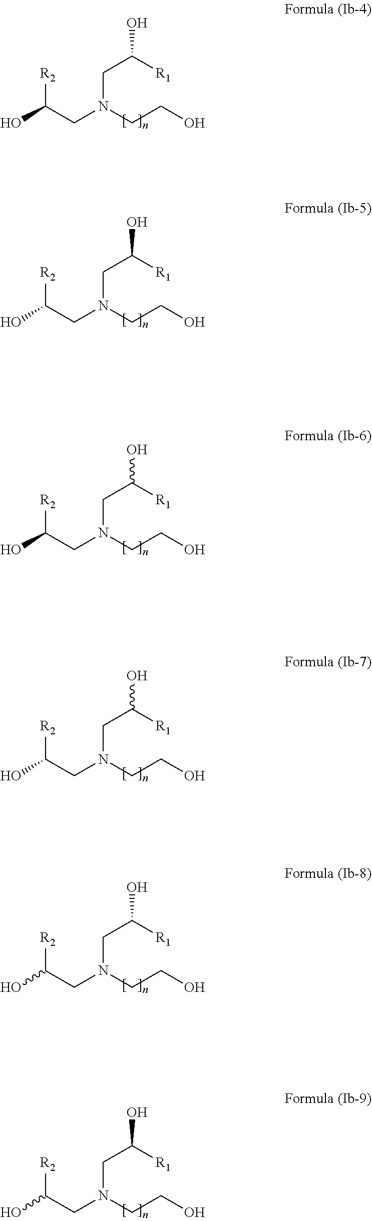

In some embodiments, the one or more of the cationic or ionizable lipids are represented by Formula (I), wherein R₁ and R₂ are each independently selected from:

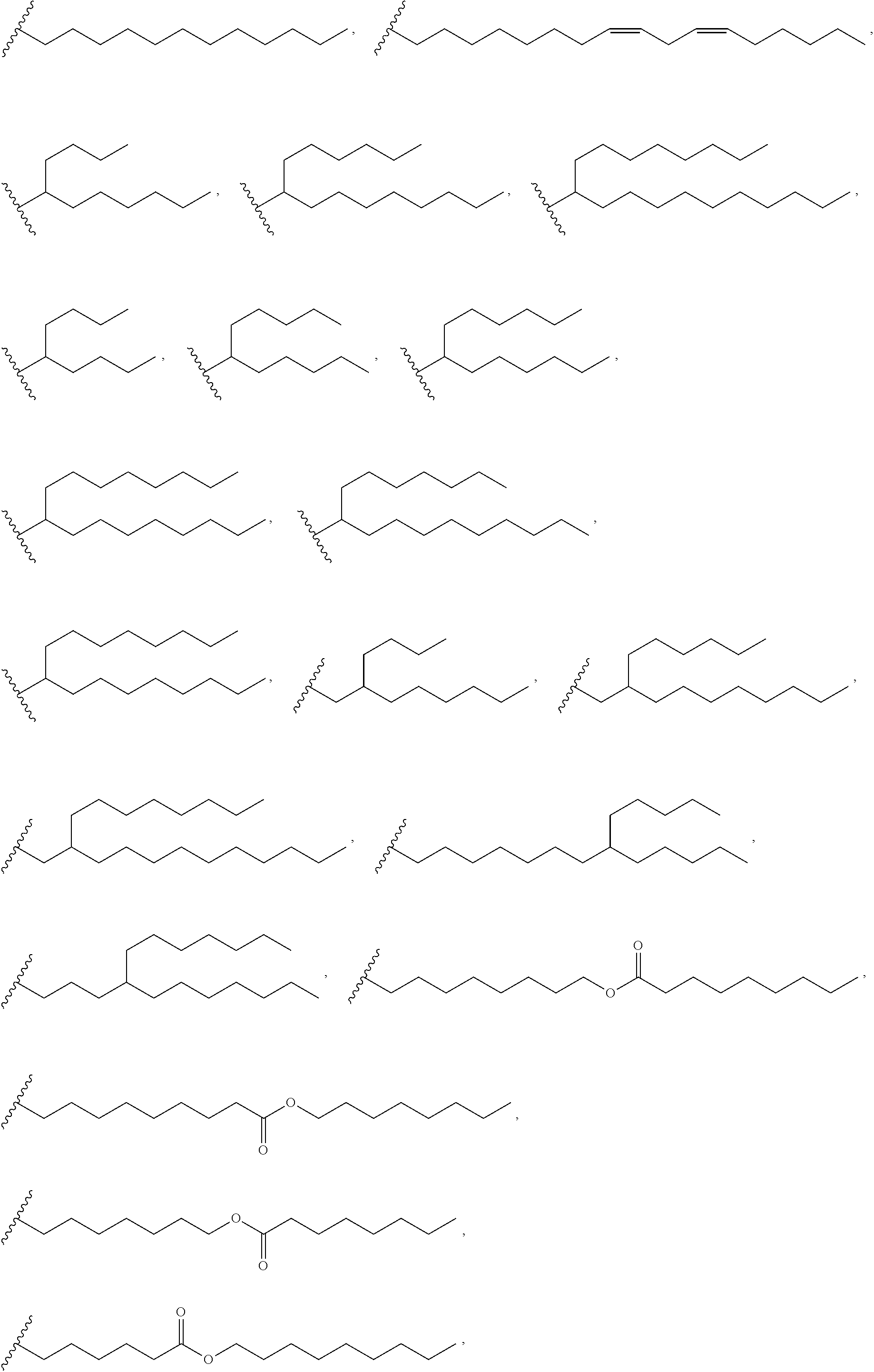

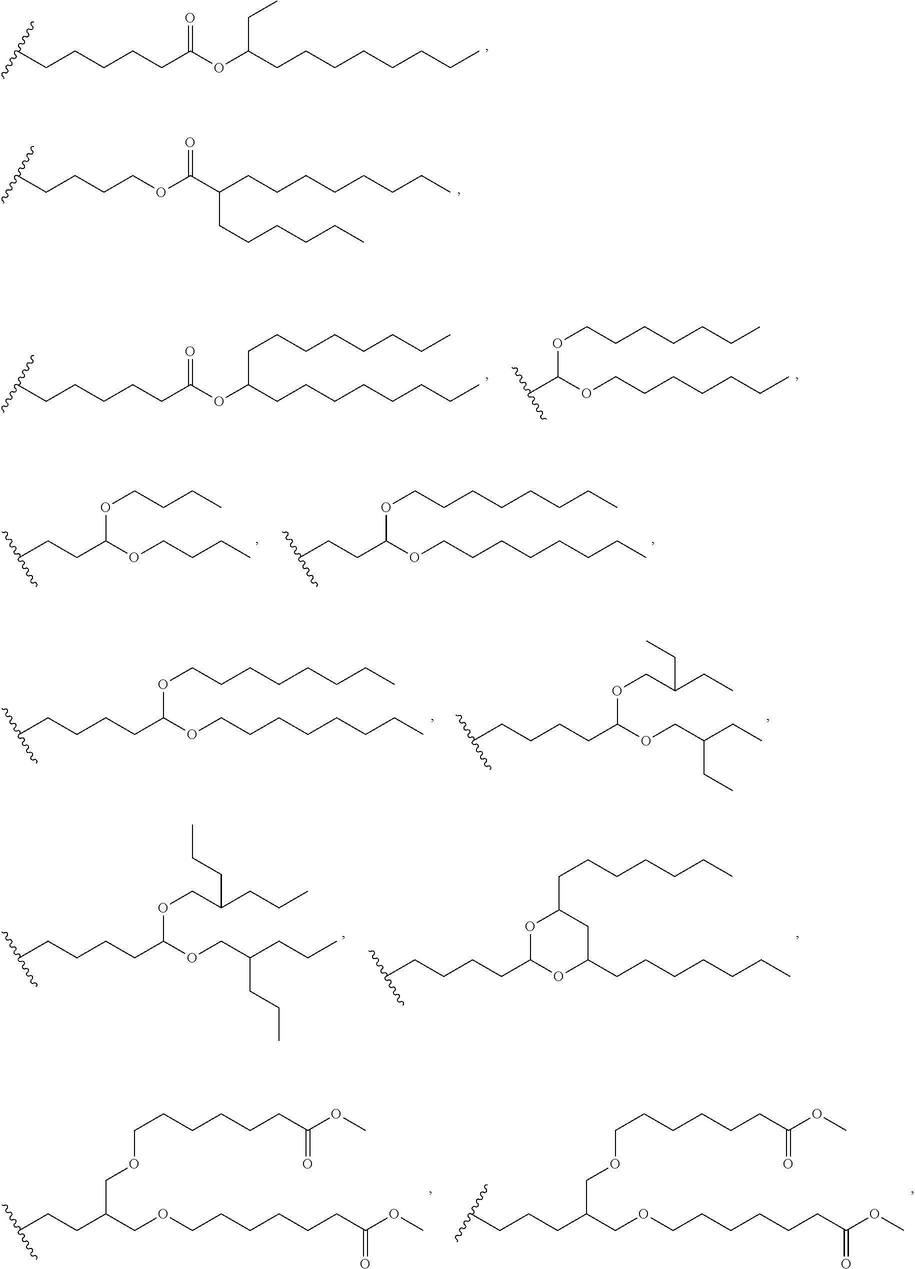

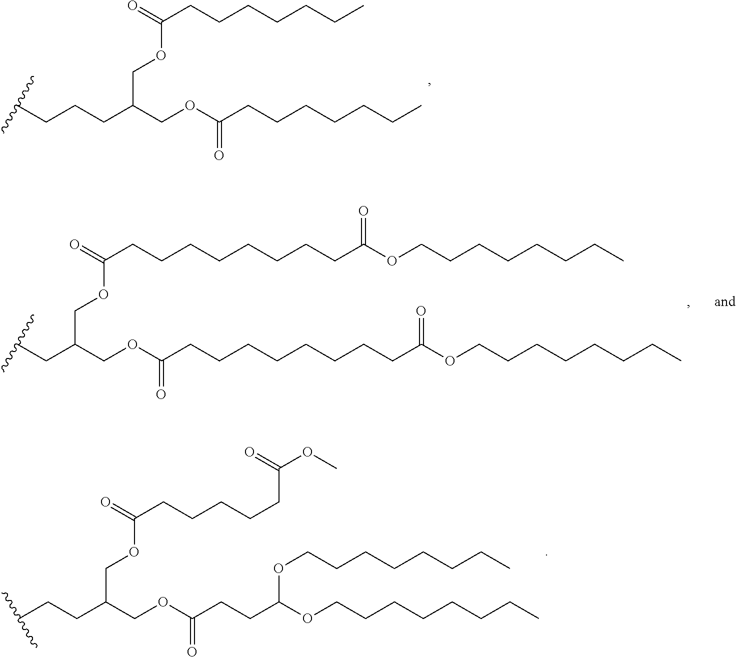 [SMILES: *CCCC(COC(=O)CCCCCCC)COC(=O)CCCCCCC ; *CC(COC(=O)CCCCCCCCC(=O)OCCCCCCCC)COC(=O)CCCCCCCCC(=O)OCCCCCCCC ; *CCC(COC(=O)CCCCCC(=O)OC)COC(=O)CCC(OCCCCCCCC)OCCCCCCCC]

In some embodiments, R₁ and R₂ are the same. In some embodiments, R, and R2 are different.

In various embodiments, the one or more of the cationic or ionizable lipids are represented by Formula (I*):

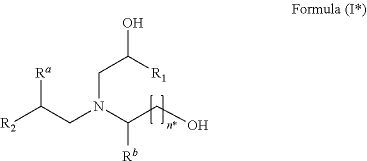

wherein:
   n* is an integer between 1 to 7,
   R^{a} is hydrogen or hydroxyl,
   R^{b} is hydrogen or C₁-C₆ alkyl,
   R₁ and R₂ are each independently a linear or branched C₁-C₃₀ alkyl, C₂-C₃₀ alkenyl, or C₁-C₃₀ heteroalkyl, optionally substituted by one or more substituents selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, alkylcarbonyloxy, alkylcarbonate, alkenyloxycarbonyl, alkenylcarbonyloxy, alkenylcarbonate, alkynyloxycarbonyl, alkynylcarbonyloxy, alkynylcarbonate, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, alkynylcarbonyl, alkylsulfoxide, alkylsulfoxidealkyl, heterocyclylcarbonyl, alkenylcarbonyl, alkylsulfonyl, and alkylsulfonealkyl.

In some embodiments, the one or more of the cationic or ionizable lipids are represented by Formula (II):

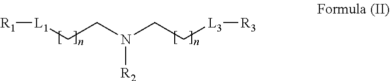

wherein:
   each n is independently an integer from 2-15;
   L₁ and L₃ are each independently —OC(O)—* or —C(O)O—*, wherein “*” indicates the attachment point to R₁ or R3;
   R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
   R₂ is selected from a group consisting of:

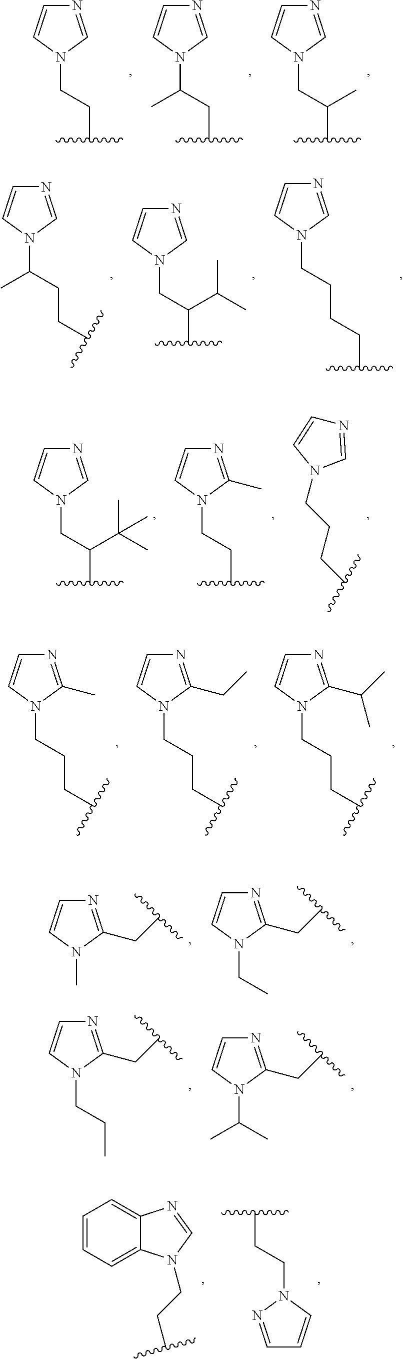

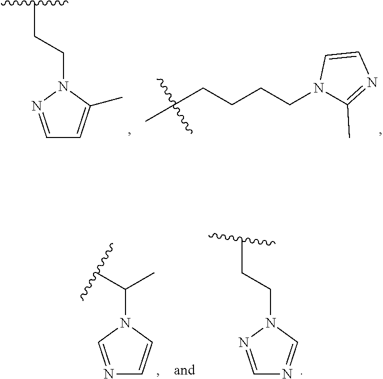

In some embodiments, the ionizable lipid is selected from an ionizable lipid of Formula II, wherein R₁ and R₃ are each independently selected from a group consisting of:

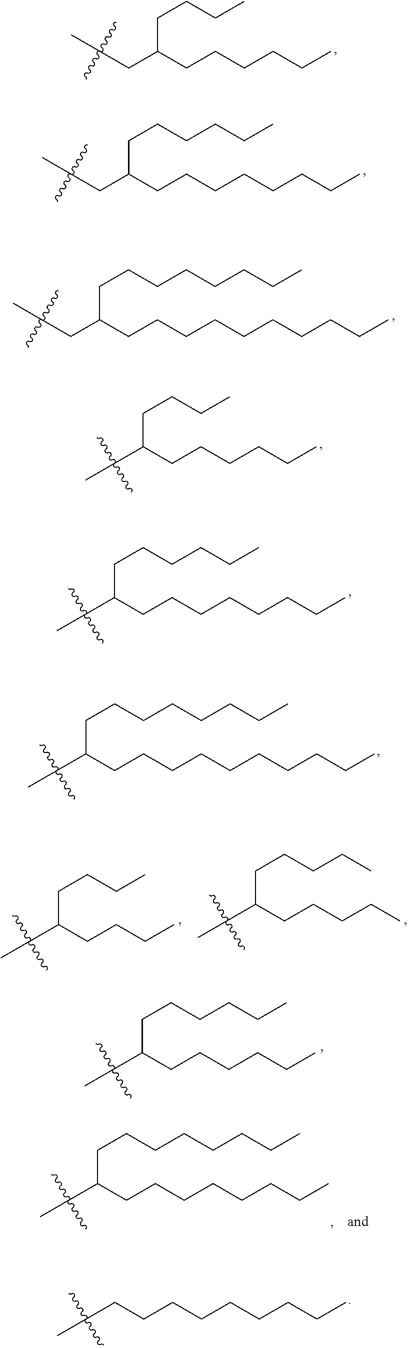

In some embodiments, R₁ and R₅ are the same. In some embodiments, R₁ and R₃ are different.

In some embodiments, the one or more of the cationic or ionizable lipids are represented by Formula (II-1) or Formula (II-2):

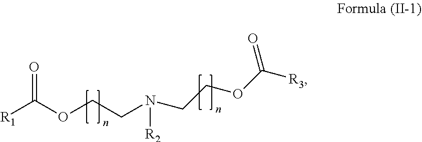

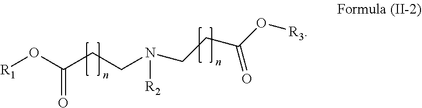

In some embodiments, the ionizable lipid is selected from an ionizable lipid of WO2015/095340 (lipid number 123 of Table 3). In some embodiments, the ionizable lipid is selected from an ionizable lipid of WO2021/021634, WO2020/237227, or WO2019/236673 (lipid numbers 124-127 of Table 3). In some embodiments, the ionizable lipid is selected from an ionizable lipid of WO2021226597 and WO2021113777 (lipid numbers 128-131 of Table 3).

In some embodiments, the transfer vehicle comprises an ionizable lipid selected from an ionizable lipid represented in Table 3. In particular embodiments, where the ionizable lipid is of Formula I, the ionizable lipid is selected from lipid numbers 16, 45, 86, 89, and 90 of Table 3, below. In particular embodiments where the ionizable lipid is an ionizable lipid of Formula II, the ionizable lipid is selected from lipid numbers 128-131 of Table 3, below.

Example 2 describes exemplary methods of synthesizing certain ionizable lipids that are represented by Formula I and II and described in Table 3.

In some embodiments, the one or more of the cationic or ionizable lipids are represented by Formula (III):

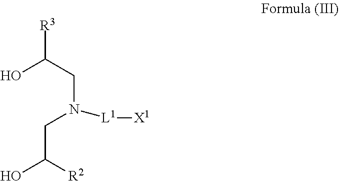

or a pharmaceutically acceptable salt thereof, wherein
   L¹ is C₂-C₁₁ alkylene, C₄-C₁₀-alkenylene, or C₄-C₁₀-alkynylene;
   X¹ is OR¹, SR¹, or N(R¹)₂, where R¹ is independently H or unsubstituted C₁-C₆ alkyl; and
   R² and R³ are each independently C₆-C₃₀-alkyl, C₆-C₃₀-alkenyl, or C₆-C₃₀-alkynyl.

In some embodiments, the one or more of the cationic or ionizable lipids are represented by Formula (III*):

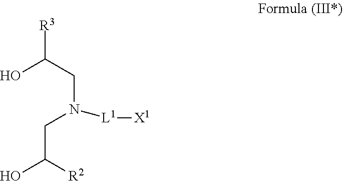

or a pharmaceutically acceptable salt thereof, wherein
   L¹ is C₂-C₁₁ alkylene, C₄-C₁₀-alkenylene, or C₄-C₁₀-alkynylene;
   X¹ is OR¹, SR¹, or N(R¹)₂, where R¹ is independently H or unsubstituted C₁-C₆ alkyl; and
   R₂ and R₃ are each independently a linear or branched C₁-C₃₀ alkyl, C₂-C₃₀ alkenyl, or C₁-C₃₀ heteroalkyl, optionally substituted by one or more substituents selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, alkylcarbonyloxy, alkylcarbonate, alkenyloxycarbonyl, alkenylcarbonyloxy, alkenylcarbonate, alkynyloxycarbonyl, alkynylcarbonyloxy, alkynylcarbonate, aminocarbonyl, aminoalkylaminocarbonyl, alkylaminoalkylaminocarbonyl, heterocyclylalkylaminocarbonyl, dialkylaminoalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl.

TABLE 3
Exemplary Ionizable Lipid Structures

| Ioniz- | |
|---|---|
| able | |
| lipid | |
| num- | |
| ber | Structure |
| 1 | 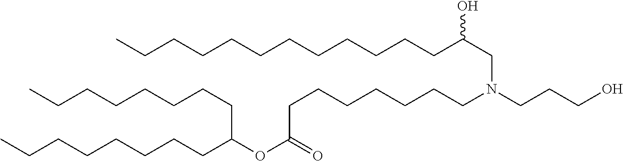 [SMILES: CCCCCCCCCCCCC(O)CN(CCCO)CCCCCCCC(=O)OC(CCCCCCCC)CCCCCCCC] |
| 2 | 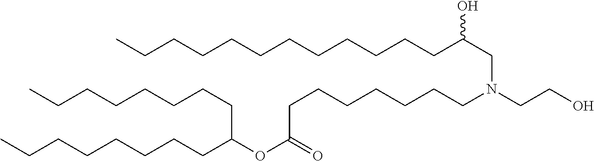 [SMILES: CCCCCCCCCCCCC(O)CN(CCO)CCCCCCCC(=O)OC(CCCCCCCC)CCCCCCCC] |
| 3 | 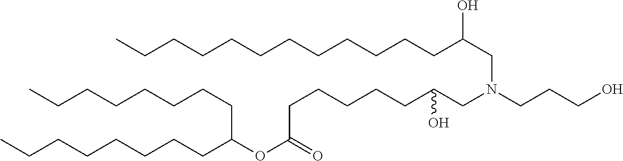 [SMILES: CCCCCCCCCCCCC(O)CN(CCCO)CC(O)CCCCCC(=O)OC(CCCCCCCC)CCCCCCCC] |
| 4 | 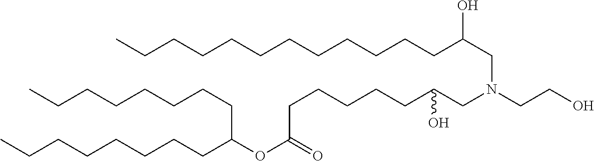 [SMILES: CCCCCCCCCCCCC(O)CN(CCO)CC(O)CCCCCC(=O)OC(CCCCCCCC)CCCCCCCC] |
| 5 | 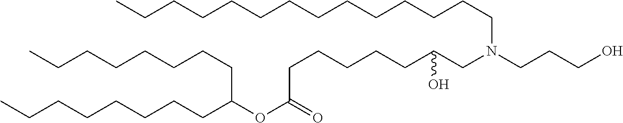 [SMILES: CCCCCCCCCCCCCCN(CCCO)CC(O)CCCCCC(=O)OC(CCCCCCCC)CCCCCCCC] |
| 6 | 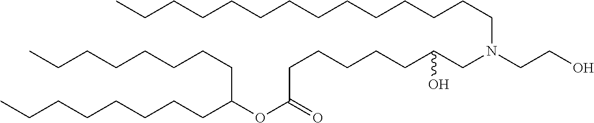 [SMILES: CCCCCCCCCCCCCCN(CCO)CC(O)CCCCCC(=O)OC(CCCCCCCC)CCCCCCCC] |
| 7 | 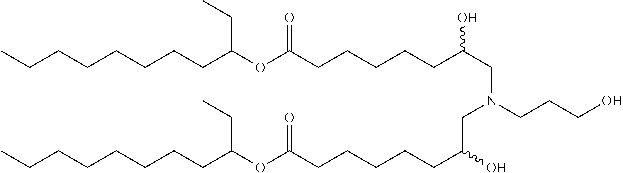 [SMILES: CCCCCCCCC(CC)OC(=O)CCCCCC(O)CN(CCCO)CC(O)CCCCCC(=O)OC(CC)CCCCCCCC] |
| 8 | 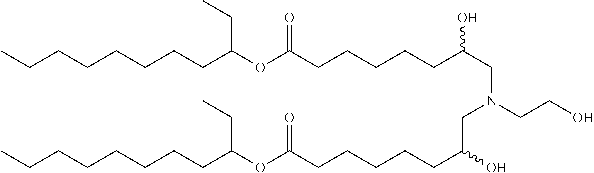 [SMILES: CCCCCCCCC(CC)OC(=O)CCCCCC(O)CN(CCO)CC(O)CCCCCC(=O)OC(CC)CCCCCCCC] |
| 9 | 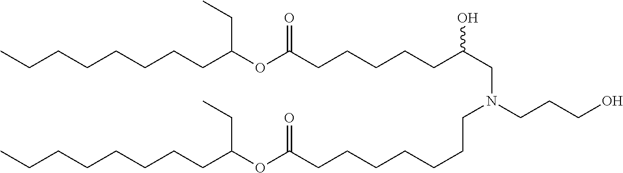 [SMILES: CCCCCCCCC(CC)OC(=O)CCCCCCCN(CCCO)CC(O)CCCCCC(=O)OC(CC)CCCCCCCC] |
| 10 | 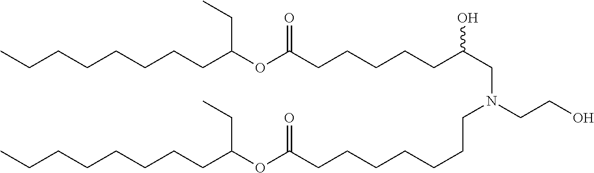 [SMILES: CCCCCCCCC(CC)OC(=O)CCCCCCCN(CCO)CC(O)CCCCCC(=O)OC(CC)CCCCCCCC] |
| 11 | 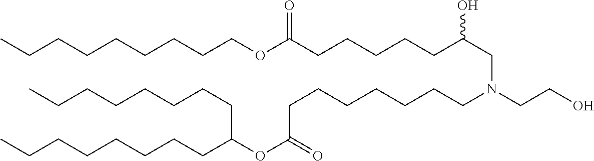 [SMILES: CCCCCCCCCOC(=O)CCCCCC(O)CN(CCO)CCCCCCCC(=O)OC(CCCCCCCC)CCCCCCCC] |
| 12 | 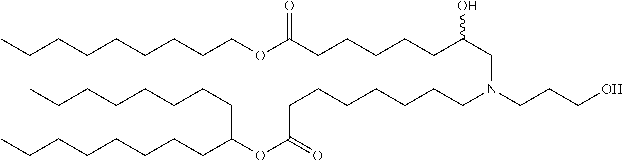 [SMILES: CCCCCCCCCOC(=O)CCCCCC(O)CN(CCCO)CCCCCCCC(=O)OC(CCCCCCCC)CCCCCCCC] |
| 13 | 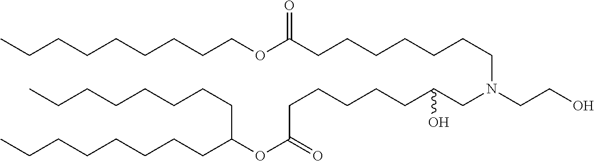 [SMILES: CCCCCCCCCOC(=O)CCCCCCCN(CCO)CC(O)CCCCCC(=O)OC(CCCCCCCC)CCCCCCCC] |
| 14 | 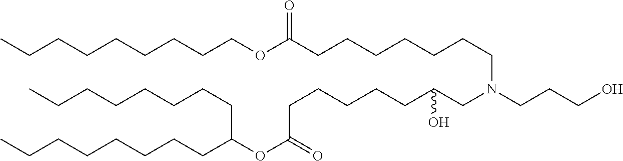 [SMILES: CCCCCCCCCOC(=O)CCCCCCCN(CCCO)CC(O)CCCCCC(=O)OC(CCCCCCCC)CCCCCCCC] |
| 15 | 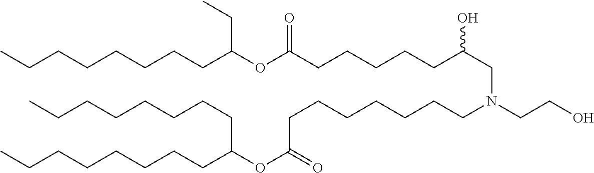 [SMILES: CCCCCCCCC(CC)OC(=O)CCCCCC(O)CN(CCO)CCCCCCCC(=O)OC(CCCCCCCC)CCCCCCCC] |
| 16 | 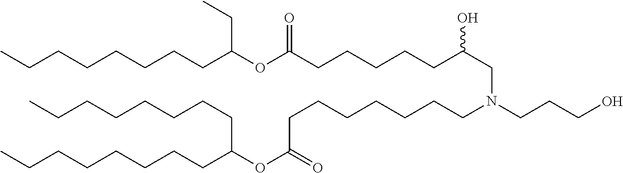 [SMILES: CCCCCCCCC(CC)OC(=O)CCCCCC(O)CN(CCCO)CCCCCCCC(=O)OC(CCCCCCCC)CCCCCCCC] |
| 17 | 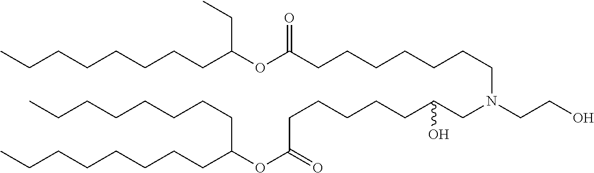 [SMILES: CCCCCCCCC(CC)OC(=O)CCCCCCCN(CCO)CC(O)CCCCCC(=O)OC(CCCCCCCC)CCCCCCCC] |
| 18 | 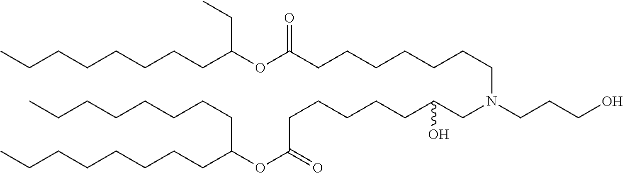 [SMILES: CCCCCCCCC(CC)OC(=O)CCCCCCCN(CCCO)CC(O)CCCCCC(=O)OC(CCCCCCCC)CCCCCCCC] |
| 19 | 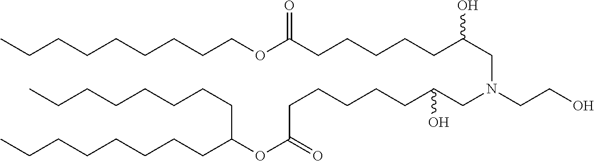 [SMILES: CCCCCCCCCOC(=O)CCCCCC(O)CN(CCO)CC(O)CCCCCC(=O)OC(CCCCCCCC)CCCCCCCC] |
| 20 | 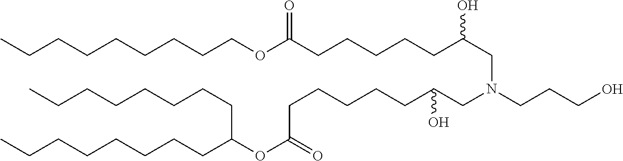 [SMILES: CCCCCCCCCOC(=O)CCCCCC(O)CN(CCCO)CC(O)CCCCCC(=O)OC(CCCCCCCC)CCCCCCCC] |
| 21 | 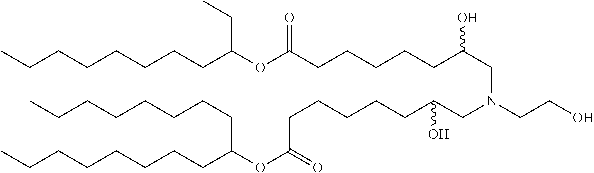 [SMILES: CCCCCCCCC(CC)OC(=O)CCCCCC(O)CN(CCO)CC(O)CCCCCC(=O)OC(CCCCCCCC)CCCCCCCC] |
| 22 | 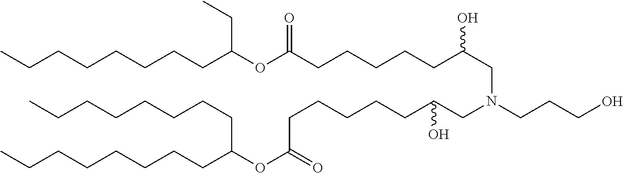 [SMILES: CCCCCCCCC(CC)OC(=O)CCCCCC(O)CN(CCCO)CC(O)CCCCCC(=O)OC(CCCCCCCC)CCCCCCCC] |
| 23 | 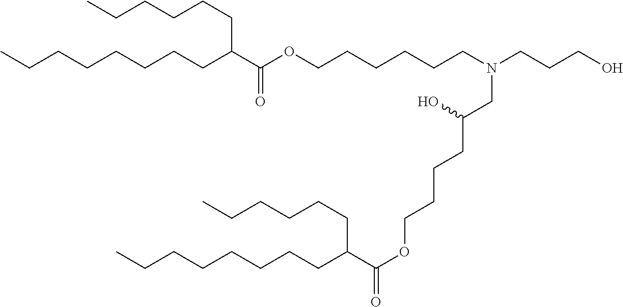 [SMILES: CCCCCCCCC(CCCCCC)C(=O)OCCCCCCN(CCCO)CC(O)CCCCOC(=O)C(CCCCCC)CCCCCCCC] |
| 24 | 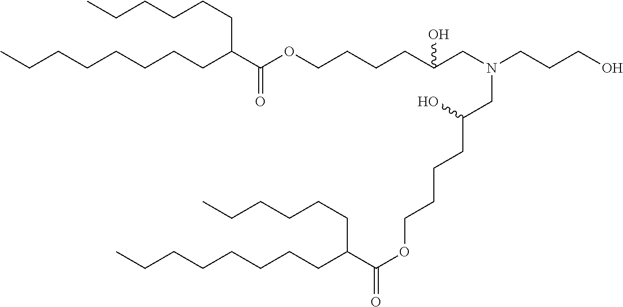 [SMILES: CCCCCCCCC(CCCCCC)C(=O)OCCCCC(O)CN(CCCO)CC(O)CCCCOC(=O)C(CCCCCC)CCCCCCCC] |
| 25 | 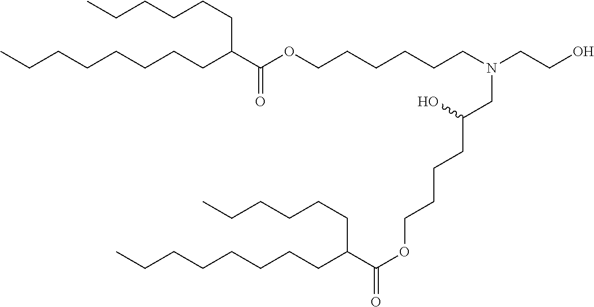 [SMILES: CCCCCCCCC(CCCCCC)C(=O)OCCCCCCN(CCO)CC(O)CCCCOC(=O)C(CCCCCC)CCCCCCCC] |
| 26 | 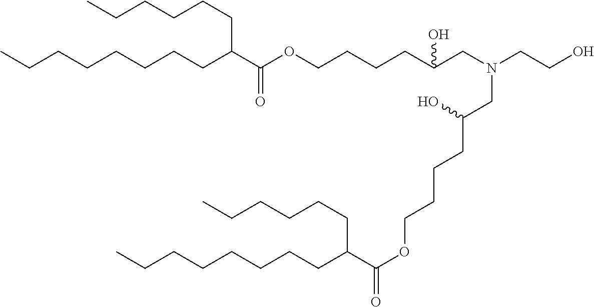 [SMILES: CCCCCCCCC(CCCCCC)C(=O)OCCCCC(O)CN(CCO)CC(O)CCCCOC(=O)C(CCCCCC)CCCCCCCC] |
| 27 | 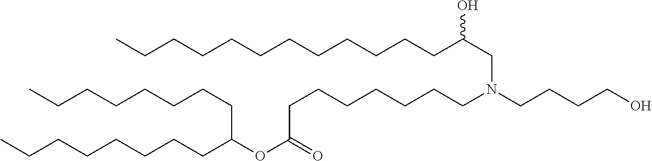 [SMILES: CCCCCCCCCCCCC(O)CN(CCCCO)CCCCCCCC(=O)OC(CCCCCCCC)CCCCCCCC] |
| 28 | 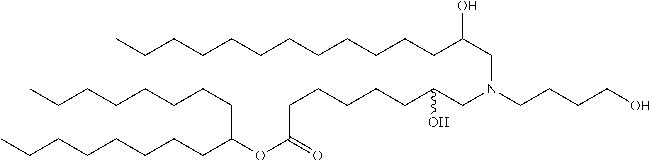 [SMILES: CCCCCCCCCCCCC(O)CN(CCCCO)CC(O)CCCCCC(=O)OC(CCCCCCCC)CCCCCCCC] |
| 29 | 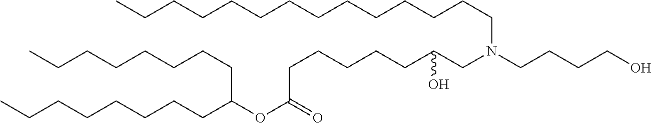 [SMILES: CCCCCCCCCCCCCCN(CCCCO)CC(O)CCCCCC(=O)OC(CCCCCCCC)CCCCCCCC] |
| 30 | 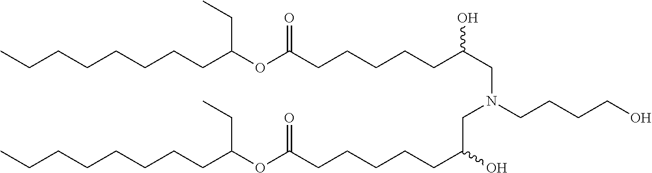 [SMILES: CCCCCCCCC(CC)OC(=O)CCCCCC(O)CN(CCCCO)CC(O)CCCCCC(=O)OC(CC)CCCCCCCC] |
| 31 | 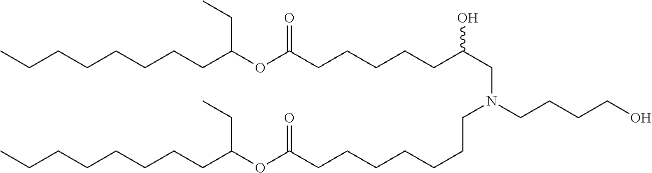 [SMILES: CCCCCCCCC(CC)OC(=O)CCCCCCCN(CCCCO)CC(O)CCCCCC(=O)OC(CC)CCCCCCCC] |
| 32 | 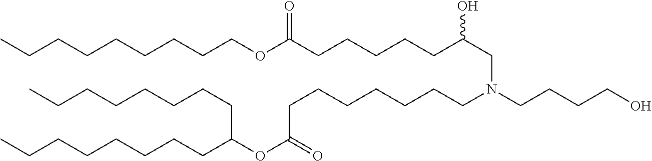 [SMILES: CCCCCCCCCOC(=O)CCCCCC(O)CN(CCCCO)CCCCCCCC(=O)OC(CCCCCCCC)CCCCCCCC] |
| 33 | 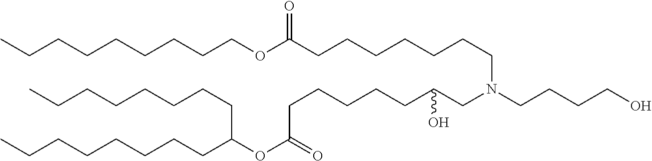 [SMILES: CCCCCCCCCOC(=O)CCCCCCCN(CCCCO)CC(O)CCCCCC(=O)OC(CCCCCCCC)CCCCCCCC] |
| 34 | 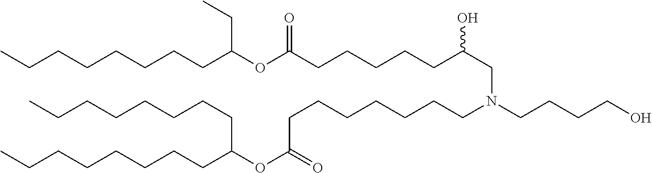 [SMILES: CCCCCCCCC(CC)OC(=O)CCCCCC(O)CN(CCCCO)CCCCCCCC(=O)OC(CCCCCCCC)CCCCCCCC] |
| 35 | 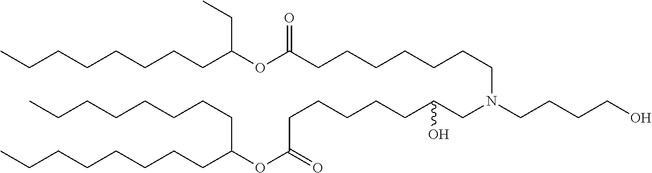 [SMILES: CCCCCCCCC(CC)OC(=O)CCCCCCCN(CCCCO)CC(O)CCCCCC(=O)OC(CCCCCCCC)CCCCCCCC] |
| 36 | 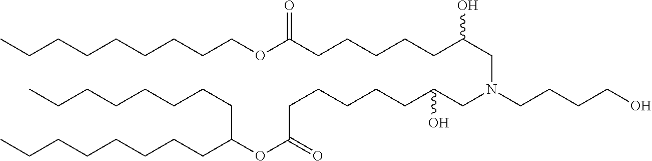 [SMILES: CCCCCCCCCOC(=O)CCCCCC(O)CN(CCCCO)CC(O)CCCCCC(=O)OC(CCCCCCCC)CCCCCCCC] |
| 37 | 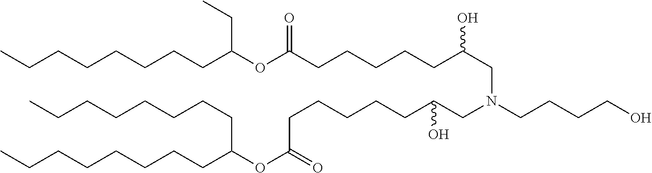 [SMILES: CCCCCCCCC(CC)OC(=O)CCCCCC(O)CN(CCCCO)CC(O)CCCCCC(=O)OC(CCCCCCCC)CCCCCCCC] |
| 38 | 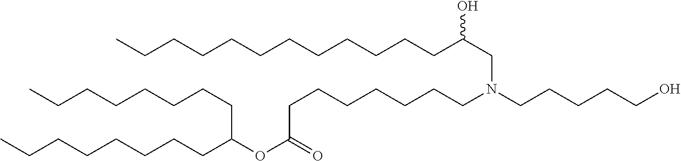 [SMILES: CCCCCCCCCCCCC(O)CN(CCCCCO)CCCCCCCC(=O)OC(CCCCCCCC)CCCCCCCC] |
| 39 | 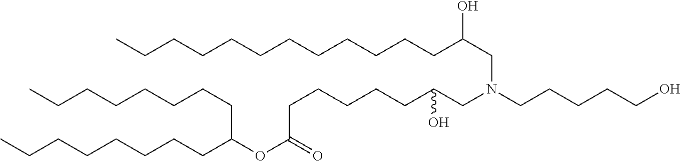 [SMILES: CCCCCCCCCCCCC(O)CN(CCCCCO)CC(O)CCCCCC(=O)OC(CCCCCCCC)CCCCCCCC] |
| 40 | 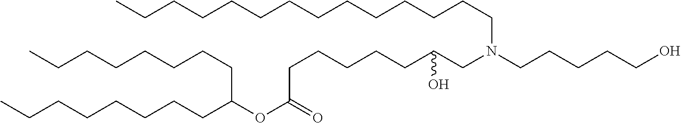 [SMILES: CCCCCCCCCCCCCCN(CCCCCO)CC(O)CCCCCC(=O)OC(CCCCCCCC)CCCCCCCC] |
| 41 | 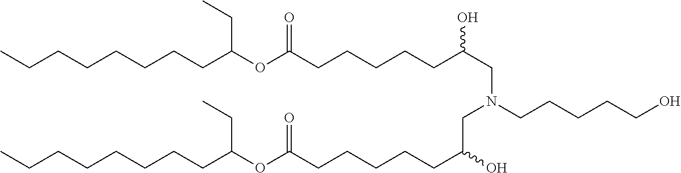 [SMILES: CCCCCCCCC(CC)OC(=O)CCCCCC(O)CN(CCCCCO)CC(O)CCCCCC(=O)OC(CC)CCCCCCCC] |
| 42 | 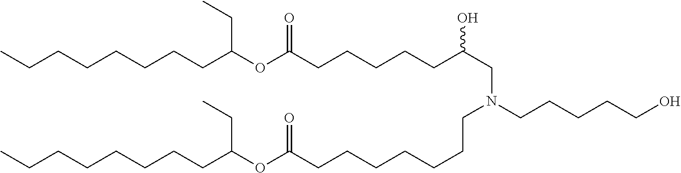 [SMILES: CCCCCCCCC(CC)OC(=O)CCCCCCCN(CCCCCO)CC(O)CCCCCC(=O)OC(CC)CCCCCCCC] |
| 43 | 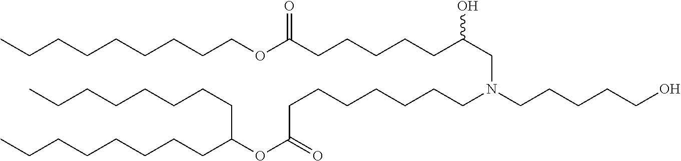 [SMILES: CCCCCCCCCOC(=O)CCCCCC(O)CN(CCCCCO)CCCCCCCC(=O)OC(CCCCCCCC)CCCCCCCC] |
| 44 | 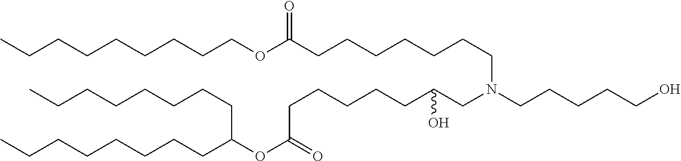 [SMILES: CCCCCCCCCOC(=O)CCCCCCCN(CCCCCO)CC(O)CCCCCC(=O)OC(CCCCCCCC)CCCCCCCC] |
| 45 | 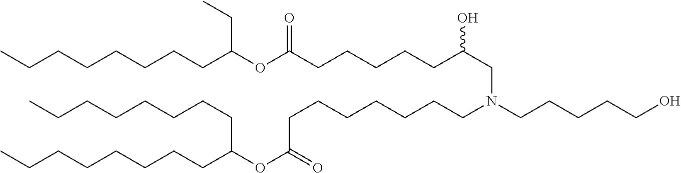 [SMILES: CCCCCCCCC(CC)OC(=O)CCCCCC(O)CN(CCCCCO)CCCCCCCC(=O)OC(CCCCCCCC)CCCCCCCC] |
| 46 | 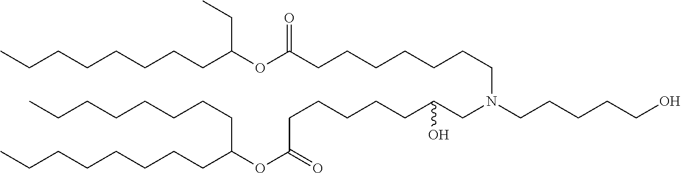 [SMILES: CCCCCCCCC(CC)OC(=O)CCCCCCCN(CCCCCO)CC(O)CCCCCC(=O)OC(CCCCCCCC)CCCCCCCC] |
| 47 | 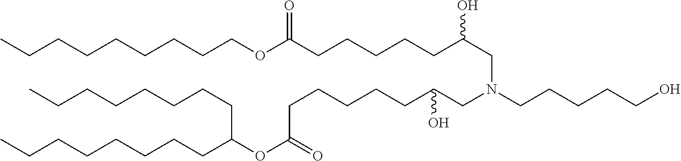 [SMILES: CCCCCCCCCOC(=O)CCCCCC(O)CN(CCCCCO)CC(O)CCCCCC(=O)OC(CCCCCCCC)CCCCCCCC] |
| 48 | 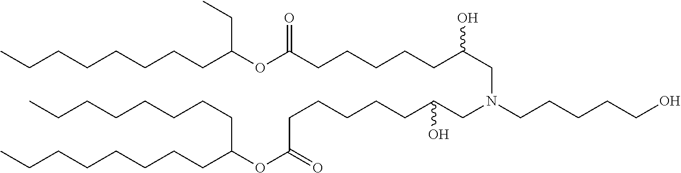 [SMILES: CCCCCCCCC(CC)OC(=O)CCCCCC(O)CN(CCCCCO)CC(O)CCCCCC(=O)OC(CCCCCCCC)CCCCCCCC] |
| 49 | 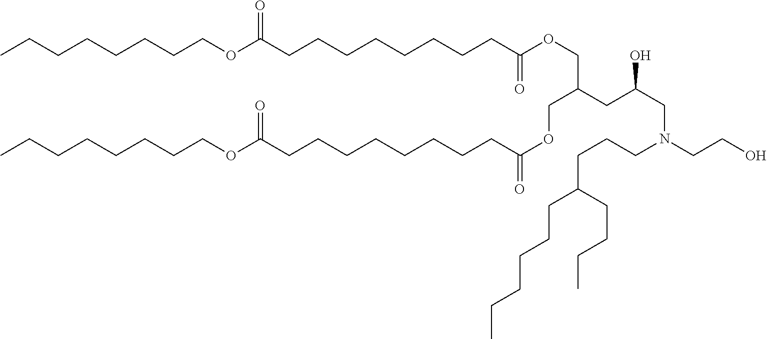 [SMILES: CCCCCCCCOC(=O)CCCCCCCCC(=O)OCC(COC(=O)CCCCCCCCC(=O)OCCCCCCCC)C[C@@H](O)CN(CCO)CCCC(CCCC)CCCCCC] |
| 50 | 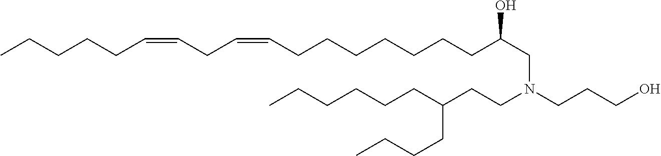 [SMILES: CCCCC/C=C\C/C=C\CCCCCCC[C@@H](O)CN(CCCO)CCC(CCCC)CCCCCC] |
| 51 | 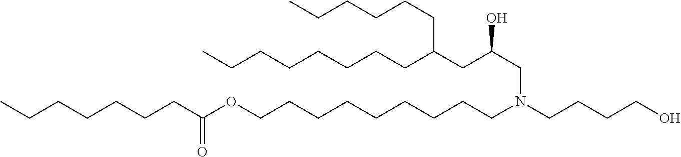 [SMILES: CCCCCCCCC(CCCCCC)C[C@@H](O)CN(CCCCO)CCCCCCCCCOC(=O)CCCCCCC] |
| 52 | 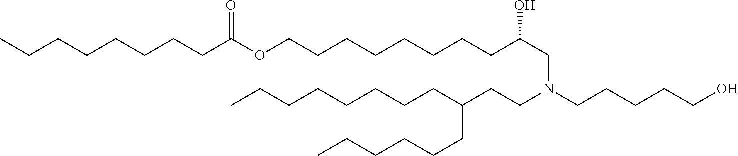 [SMILES: CCCCCCCCC(=O)OCCCCCCCC[C@H](O)CN(CCCCCO)CCC(CCCCCC)CCCCCCCC] |
| 53 | 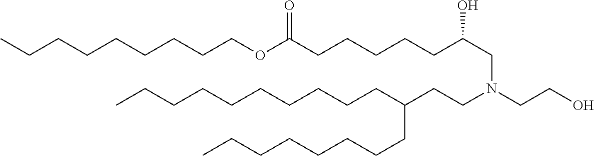 [SMILES: CCCCCCCCCCC(CCCCCCCC)CCN(CCO)C[C@@H](O)CCCCCC(=O)OCCCCCCCCC] |
| 54 | 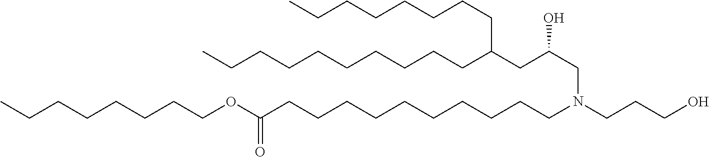 [SMILES: CCCCCCCCCCC(CCCCCCCC)C[C@H](O)CN(CCCO)CCCCCCCCCCC(=O)OCCCCCCCC] |
| 55 | 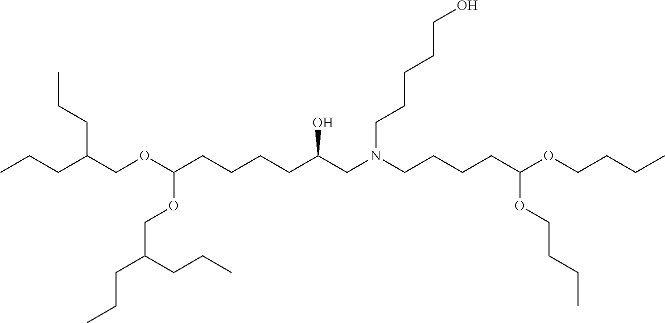 [SMILES: CCCCOC(CCCCN(CCCCCO)C[C@H](O)CCCCC(OCC(CCC)CCC)OCC(CCC)CCC)OCCCC] |
| 56 | 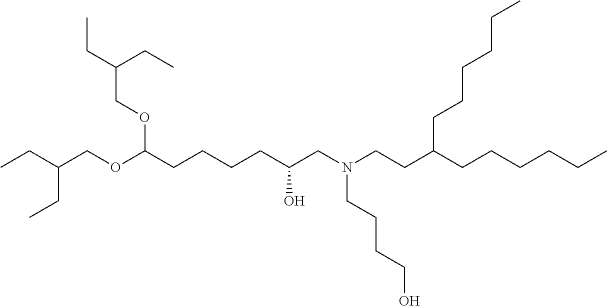 [SMILES: CCCCCCC(CCCCCC)CCN(CCCCO)C[C@H](O)CCCCC(OCC(CC)CC)OCC(CC)CC] |
| 57 | 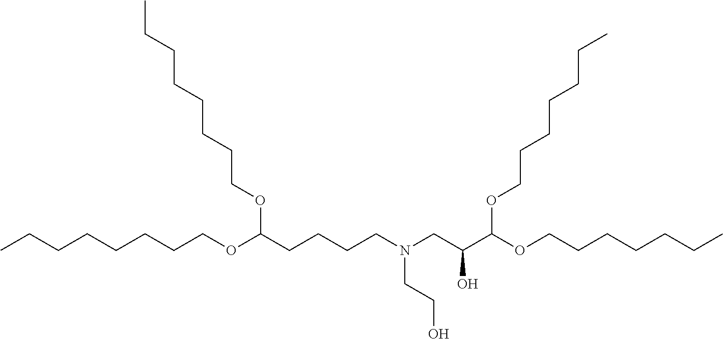 [SMILES: CCCCCCCCOC(CCCCN(CCO)C[C@H](O)C(OCCCCCCC)OCCCCCCC)OCCCCCCCC] |
| 58 | 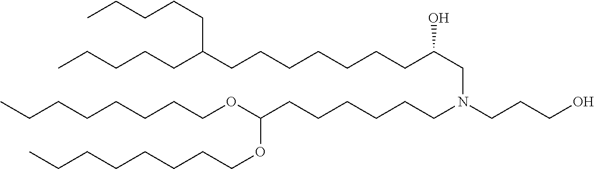 [SMILES: CCCCCCCCOC(CCCCCCN(CCCO)C[C@@H](O)CCCCCCCC(CCCCC)CCCCC)OCCCCCCCC] |
| 59 | 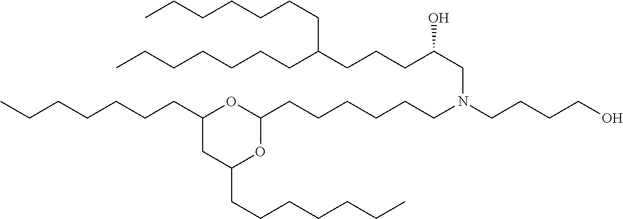 [SMILES: CCCCCCCC(CCCCCCC)CCC[C@H](O)CN(CCCCO)CCCCCCC1OC(CCCCCCC)CC(CCCCCCC)O1] |
| 60 | 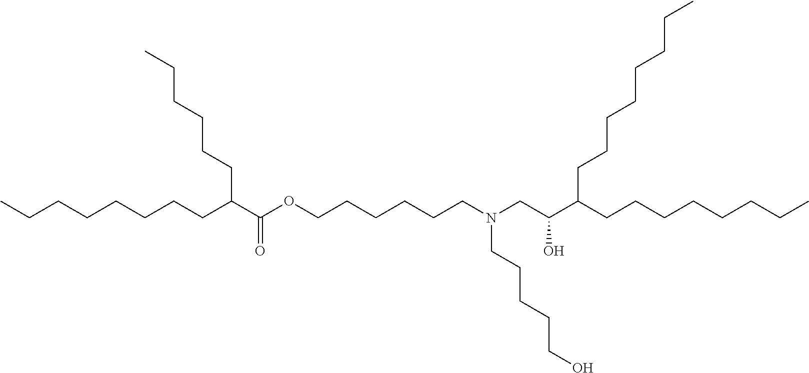 [SMILES: CCCCCCCCC(CCCCCC)C(=O)OCCCCCCN(CCCCCO)C[C@@H](O)C(CCCCCCCC)CCCCCCCC] |
| 61 | 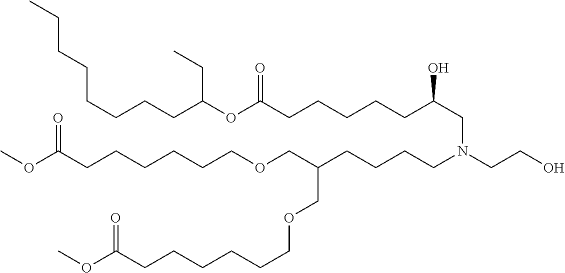 [SMILES: CCCCCCCCC(CC)OC(=O)CCCCC[C@@H](O)CN(CCO)CCCCC(COCCCCCCC(=O)OC)COCCCCCCC(=O)OC] |
| 62 | 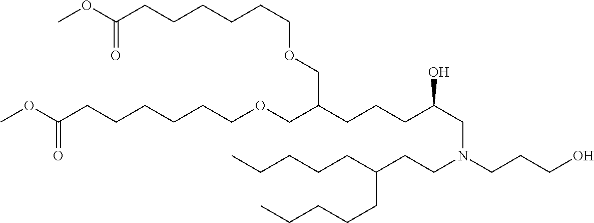 [SMILES: CCCCCC(CCCCC)CCN(CCCO)C[C@H](O)CCCC(COCCCCCCC(=O)OC)COCCCCCCC(=O)OC] |
| 63 | 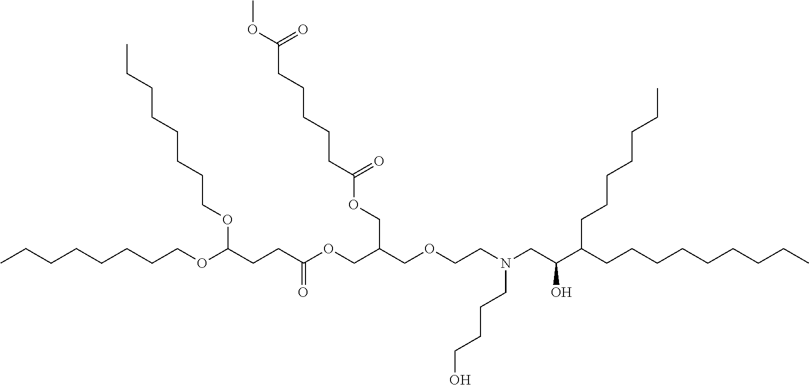 [SMILES: CCCCCCCCCC(CCCCCCC)[C@@H](O)CN(CCCCO)CCOCC(COC(=O)CCCCCC(=O)OC)COC(=O)CCC(OCCCCCCCC)OCCCCCCCC] |
| 64 | 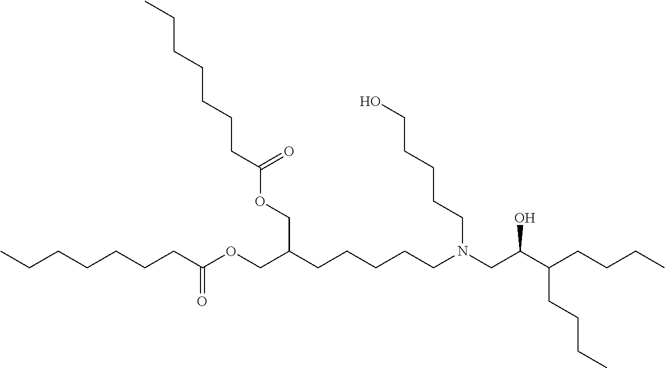 [SMILES: CCCCCCCC(=O)OCC(CCCCCN(CCCCCO)C[C@@H](O)C(CCCC)CCCC)COC(=O)CCCCCCC] |
| 65 | 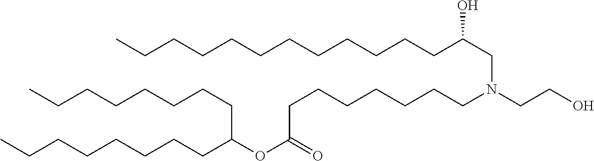 [SMILES: CCCCCCCCCCCC[C@H](O)CN(CCO)CCCCCCCC(=O)OC(CCCCCCCC)CCCCCCCC] |
| 66 | 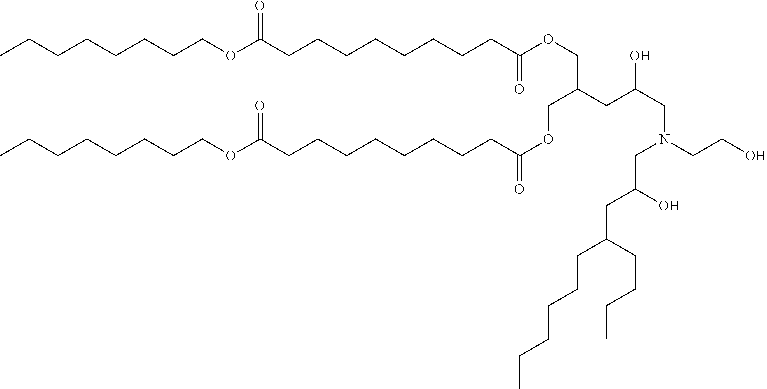 [SMILES: CCCCCCCCOC(=O)CCCCCCCCC(=O)OCC(COC(=O)CCCCCCCCC(=O)OCCCCCCCC)CC(O)CN(CCO)CC(O)CC(CCCC)CCCCCC] |
| 67 | 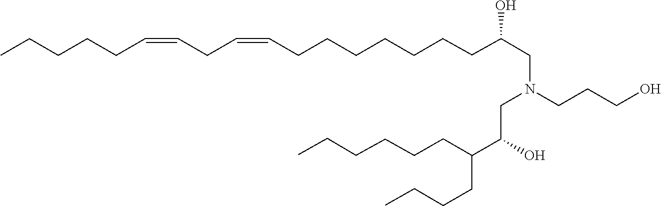 [SMILES: CCCCC/C=C\C/C=C\CCCCCCC[C@H](O)CN(CCCO)C[C@H](O)C(CCCC)CCCCCC] |
| 68 | 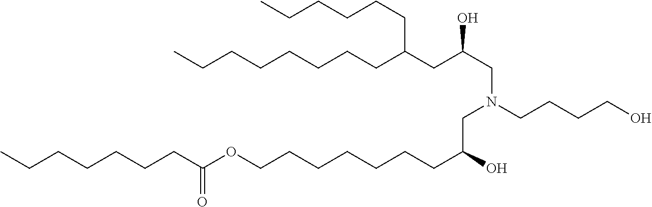 [SMILES: CCCCCCCCC(CCCCCC)C[C@@H](O)CN(CCCCO)C[C@@H](O)CCCCCCCOC(=O)CCCCCCC] |
| 69 | 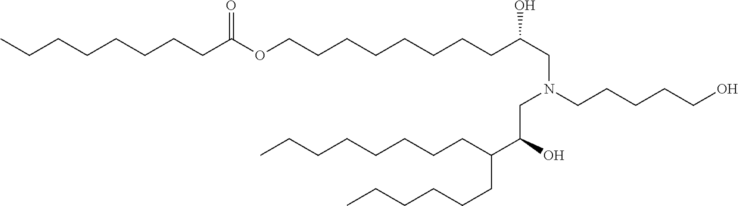 [SMILES: CCCCCCCCC(=O)OCCCCCCCC[C@H](O)CN(CCCCCO)C[C@@H](O)C(CCCCCC)CCCCCCCC] |
| 70 | 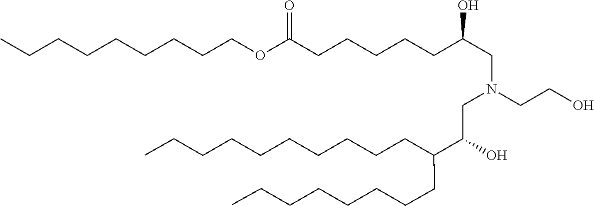 [SMILES: CCCCCCCCCCC(CCCCCCCC)[C@@H](O)CN(CCO)C[C@H](O)CCCCCC(=O)OCCCCCCCCC] |
| 71 | 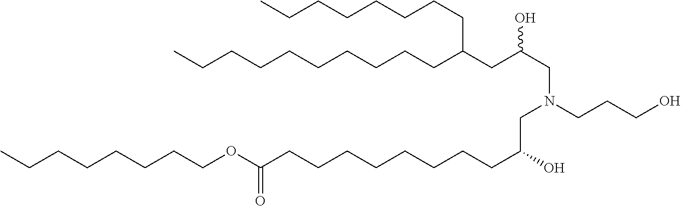 [SMILES: CCCCCCCCCCC(CCCCCCCC)CC(O)CN(CCCO)C[C@H](O)CCCCCCCCC(=O)OCCCCCCCC] |
| 72 | 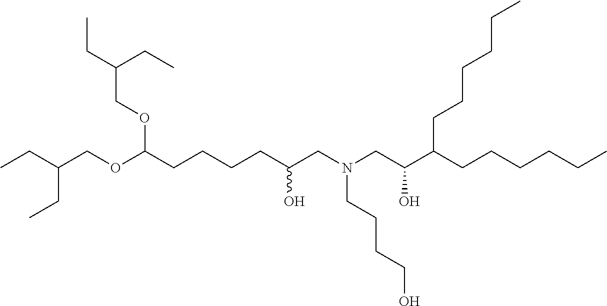 [SMILES: CCCCCCC(CCCCCC)[C@H](O)CN(CCCCO)CC(O)CCCCC(OCC(CC)CC)OCC(CC)CC] |
| 73 | 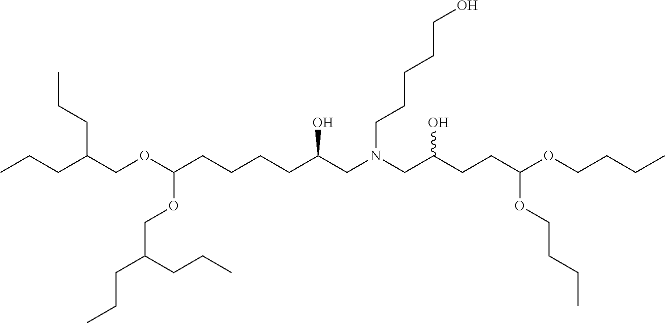 [SMILES: CCCCOC(CCC(O)CN(CCCCCO)C[C@H](O)CCCCC(OCC(CCC)CCC)OCC(CCC)CCC)OCCCC] |
| 74 | 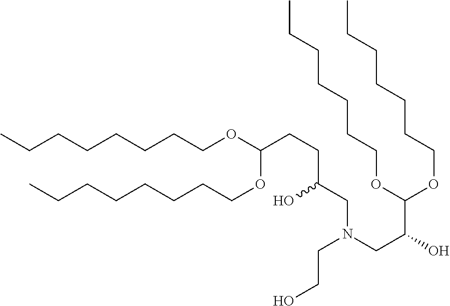 [SMILES: CCCCCCCCOC(CCC(O)CN(CCO)C[C@@H](O)C(OCCCCCCC)OCCCCCCC)OCCCCCCCC] |
| 75 | 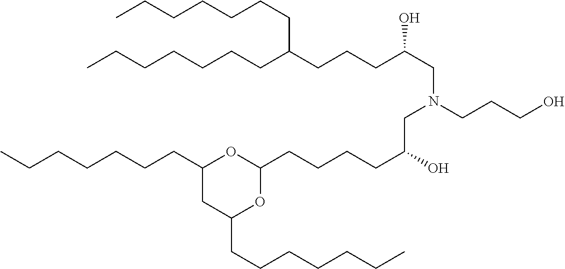 [SMILES: CCCCCCCC(CCCCCCC)CCC[C@H](O)CN(CCCO)C[C@H](O)CCCCC1OC(CCCCCCC)CC(CCCCCCC)O1] |
| 76 | 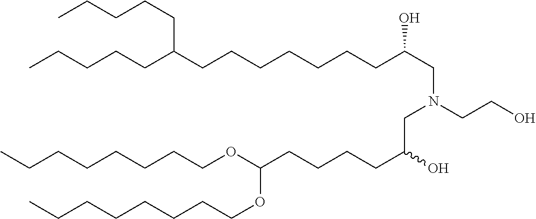 [SMILES: CCCCCCCCOC(CCCCC(O)CN(CCO)C[C@@H](O)CCCCCCCC(CCCCC)CCCCC)OCCCCCCCC] |
| 77 | 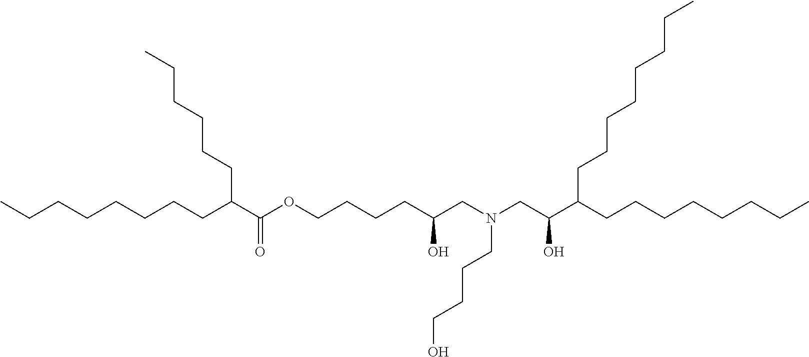 [SMILES: CCCCCCCCC(CCCCCC)C(=O)OCCCC[C@H](O)CN(CCCCO)C[C@H](O)C(CCCCCCCC)CCCCCCCC] |
| 78 | 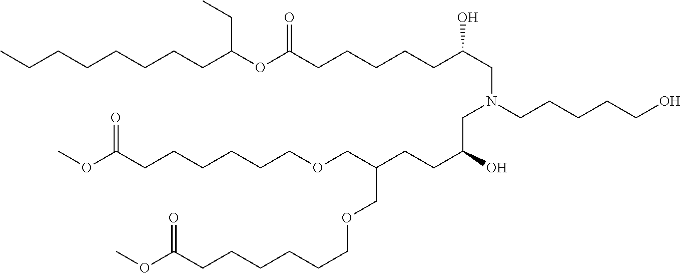 [SMILES: CCCCCCCCC(CC)OC(=O)CCCCC[C@H](O)CN(CCCCCO)C[C@@H](O)CCC(COCCCCCCC(=O)OC)COCCCCCCC(=O)OC] |
| 79 | 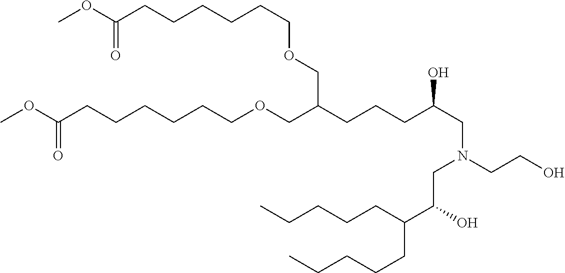 [SMILES: CCCCCC(CCCCC)[C@@H](O)CN(CCO)C[C@H](O)CCCC(COCCCCCCC(=O)OC)COCCCCCCC(=O)OC] |
| 80 | 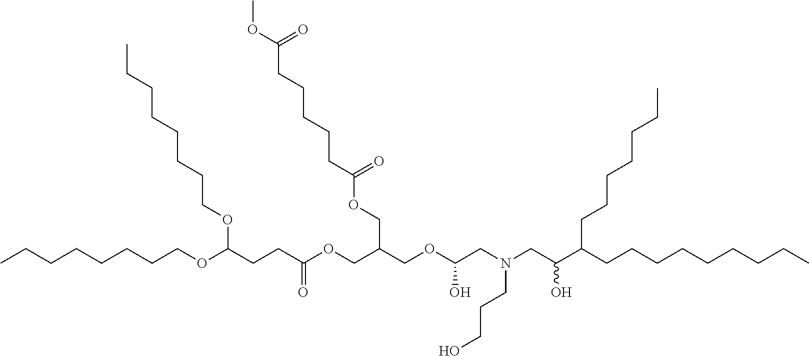 [SMILES: CCCCCCCCCC(CCCCCCC)C(O)CN(CCCO)C[C@H](O)OCC(COC(=O)CCCCCC(=O)OC)COC(=O)CCC(OCCCCCCCC)OCCCCCCCC] |
| 81 | 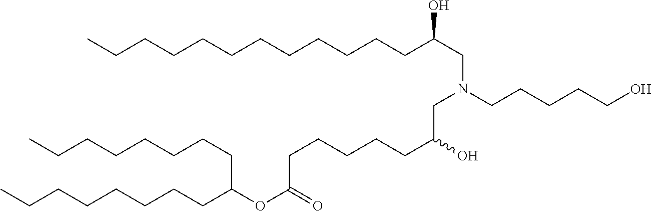 [SMILES: CCCCCCCCCCCC[C@@H](O)CN(CCCCCO)CC(O)CCCCCC(=O)OC(CCCCCCCC)CCCCCCCC] |
| 82 | 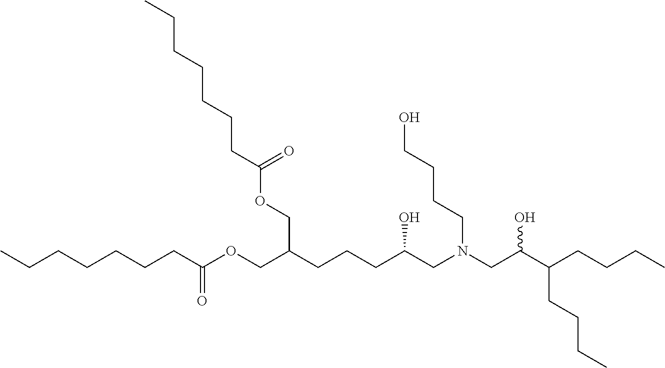 [SMILES: CCCCCCCC(=O)OCC(CCC[C@H](O)CN(CCCCO)CC(O)C(CCCC)CCCC)COC(=O)CCCCCCC] |
| 83 | 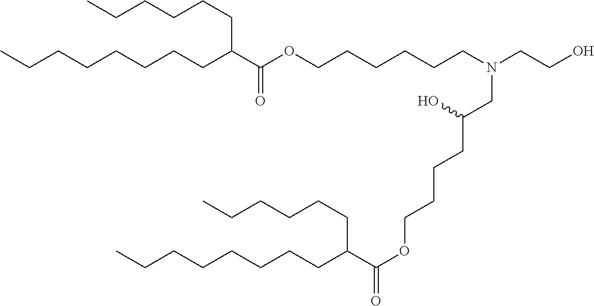 [SMILES: CCCCCCCCC(CCCCCC)C(=O)OCCCCCCN(CCO)CC(O)CCCCOC(=O)C(CCCCCC)CCCCCCCC] |
| 84 | 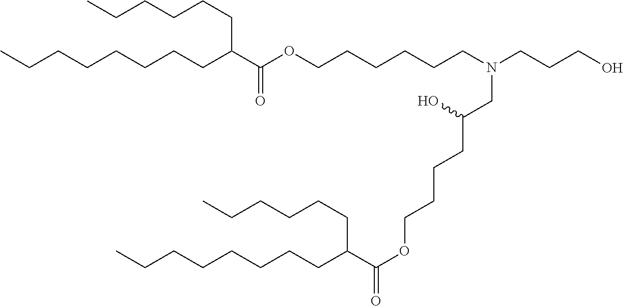 [SMILES: CCCCCCCCC(CCCCCC)C(=O)OCCCCCCN(CCCO)CC(O)CCCCOC(=O)C(CCCCCC)CCCCCCCC] |
| 85 | 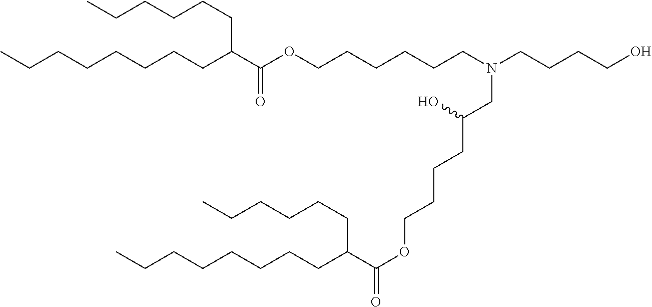 [SMILES: CCCCCCCCC(CCCCCC)C(=O)OCCCCCCN(CCCCO)CC(O)CCCCOC(=O)C(CCCCCC)CCCCCCCC] |
| 86 | 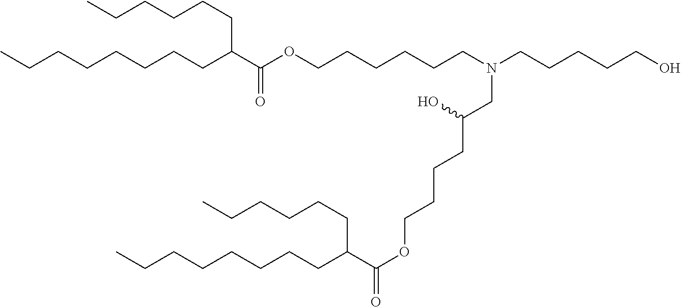 [SMILES: CCCCCCCCC(CCCCCC)C(=O)OCCCCCCN(CCCCCO)CC(O)CCCCOC(=O)C(CCCCCC)CCCCCCCC] |
| 87 | 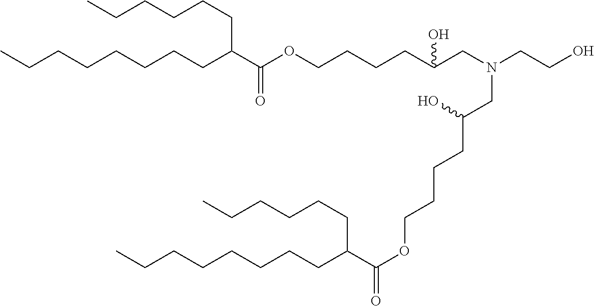 [SMILES: CCCCCCCCC(CCCCCC)C(=O)OCCCCC(O)CN(CCO)CC(O)CCCCOC(=O)C(CCCCCC)CCCCCCCC] |
| 88 | 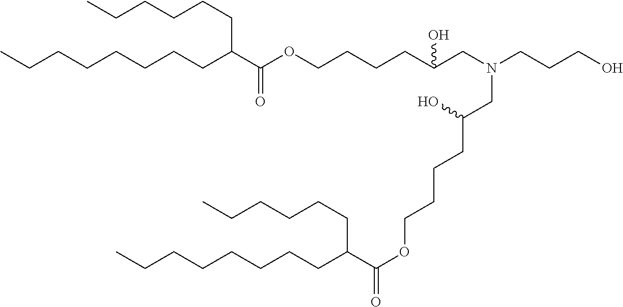 [SMILES: CCCCCCCCC(CCCCCC)C(=O)OCCCCC(O)CN(CCCO)CC(O)CCCCOC(=O)C(CCCCCC)CCCCCCCC] |
| 89 | 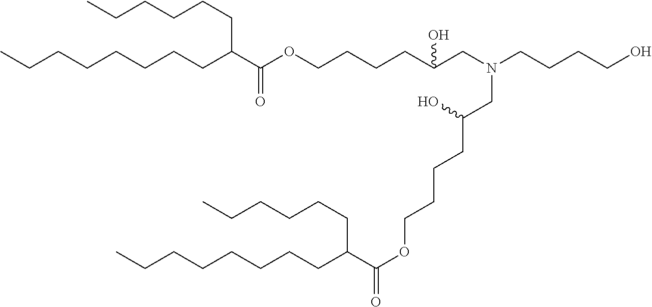 [SMILES: CCCCCCCCC(CCCCCC)C(=O)OCCCCC(O)CN(CCCCO)CC(O)CCCCOC(=O)C(CCCCCC)CCCCCCCC] |
| 90 | 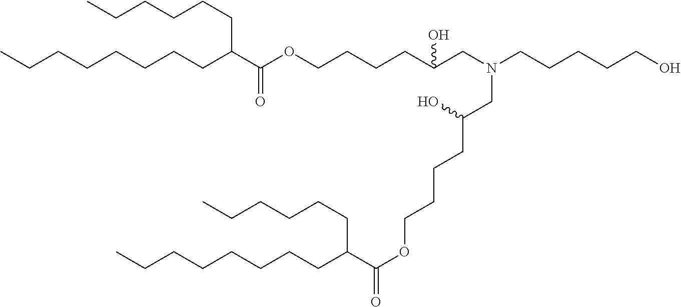 [SMILES: CCCCCCCCC(CCCCCC)C(=O)OCCCCC(O)CN(CCCCCO)CC(O)CCCCOC(=O)C(CCCCCC)CCCCCCCC] |
| 91 | 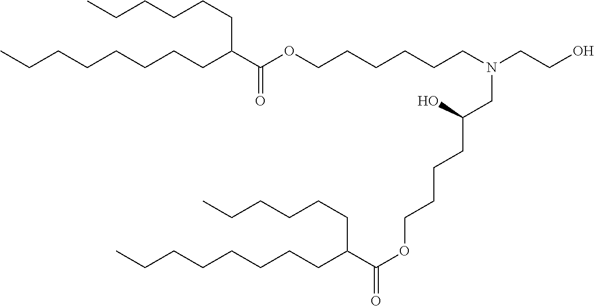 [SMILES: CCCCCCCCC(CCCCCC)C(=O)OCCCCCCN(CCO)C[C@H](O)CCCCOC(=O)C(CCCCCC)CCCCCCCC] |
| 92 | 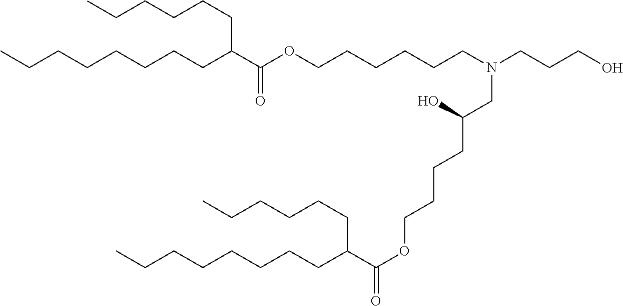 [SMILES: CCCCCCCCC(CCCCCC)C(=O)OCCCCCCN(CCCO)C[C@H](O)CCCCOC(=O)C(CCCCCC)CCCCCCCC] |
| 93 | 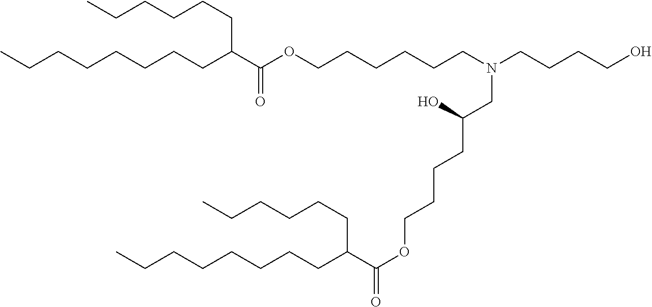 [SMILES: CCCCCCCCC(CCCCCC)C(=O)OCCCCCCN(CCCCO)C[C@H](O)CCCCOC(=O)C(CCCCCC)CCCCCCCC] |
| 94 | 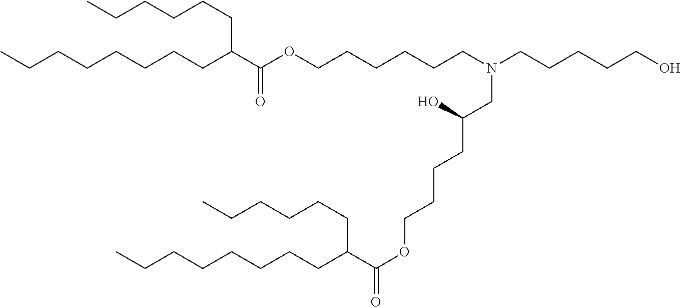 [SMILES: CCCCCCCCC(CCCCCC)C(=O)OCCCCCCN(CCCCCO)C[C@H](O)CCCCOC(=O)C(CCCCCC)CCCCCCCC] |
| 95 | 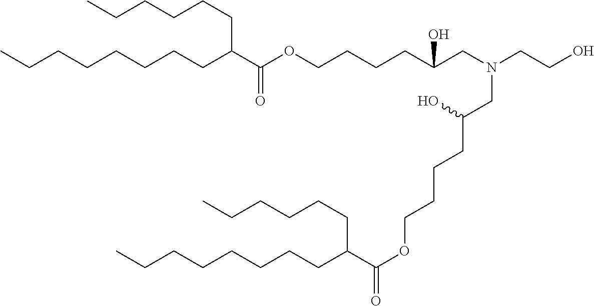 [SMILES: CCCCCCCCC(CCCCCC)C(=O)OCCCCC(O)CN(CCO)C[C@@H](O)CCCCOC(=O)C(CCCCCC)CCCCCCCC] |
| 96 | 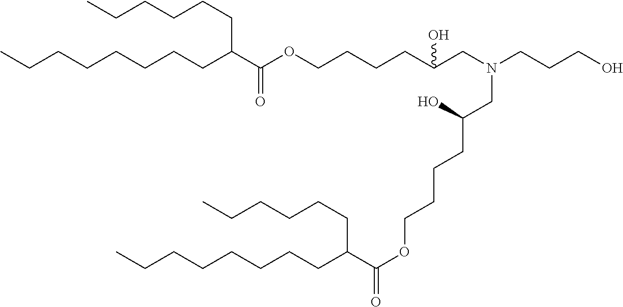 [SMILES: CCCCCCCCC(CCCCCC)C(=O)OCCCCC(O)CN(CCCO)C[C@H](O)CCCCOC(=O)C(CCCCCC)CCCCCCCC] |
| 97 | 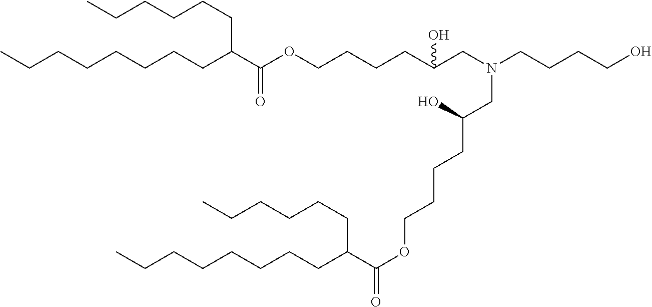 [SMILES: CCCCCCCCC(CCCCCC)C(=O)OCCCCC(O)CN(CCCCO)C[C@H](O)CCCCOC(=O)C(CCCCCC)CCCCCCCC] |
| 98 | 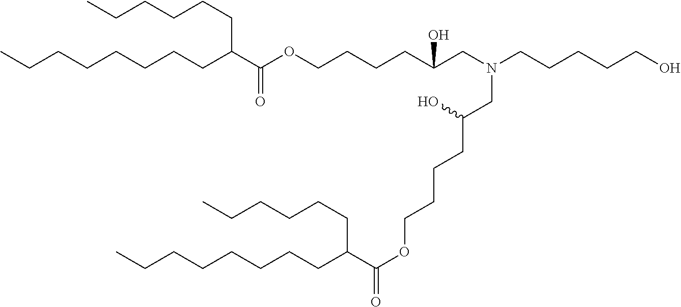 [SMILES: CCCCCCCCC(CCCCCC)C(=O)OCCCCC(O)CN(CCCCCO)C[C@@H](O)CCCCOC(=O)C(CCCCCC)CCCCCCCC] |
| 99 | 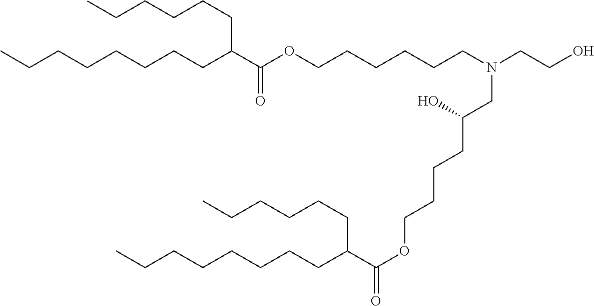 [SMILES: CCCCCCCCC(CCCCCC)C(=O)OCCCCCCN(CCO)C[C@@H](O)CCCCOC(=O)C(CCCCCC)CCCCCCCC] |
| 100 | 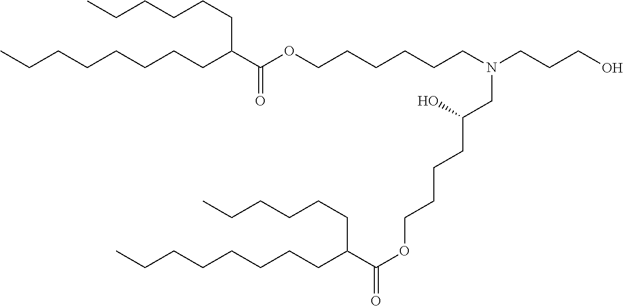 [SMILES: CCCCCCCCC(CCCCCC)C(=O)OCCCCCCN(CCCO)C[C@@H](O)CCCCOC(=O)C(CCCCCC)CCCCCCCC] |
| 101 | 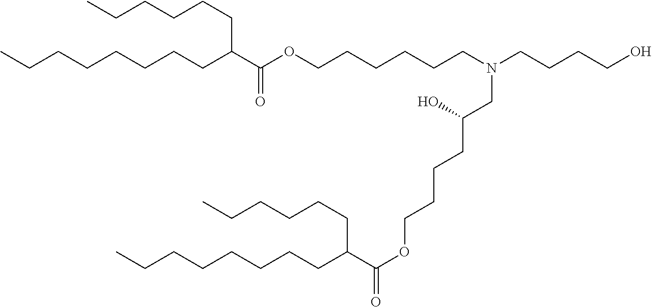 [SMILES: CCCCCCCCC(CCCCCC)C(=O)OCCCCCCN(CCCCO)C[C@@H](O)CCCCOC(=O)C(CCCCCC)CCCCCCCC] |
| 102 | 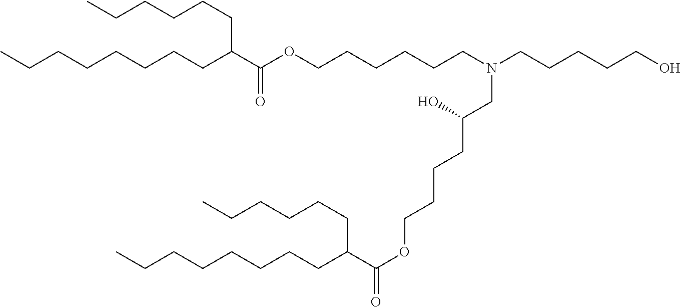 [SMILES: CCCCCCCCC(CCCCCC)C(=O)OCCCCCCN(CCCCCO)C[C@@H](O)CCCCOC(=O)C(CCCCCC)CCCCCCCC] |
| 103 | 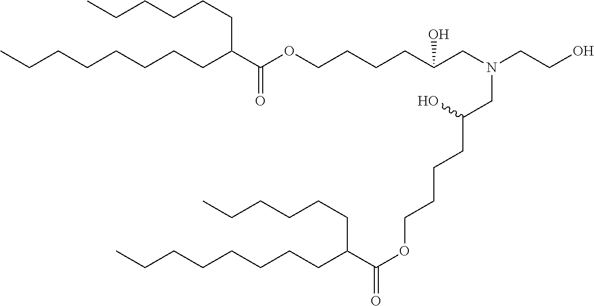 [SMILES: CCCCCCCCC(CCCCCC)C(=O)OCCCCC(O)CN(CCO)C[C@H](O)CCCCOC(=O)C(CCCCCC)CCCCCCCC] |
| 104 | 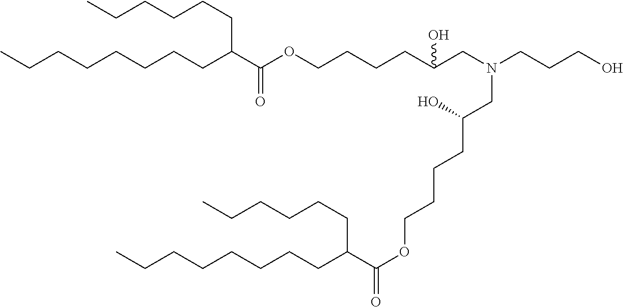 [SMILES: CCCCCCCCC(CCCCCC)C(=O)OCCCCC(O)CN(CCCO)C[C@@H](O)CCCCOC(=O)C(CCCCCC)CCCCCCCC] |
| 105 | 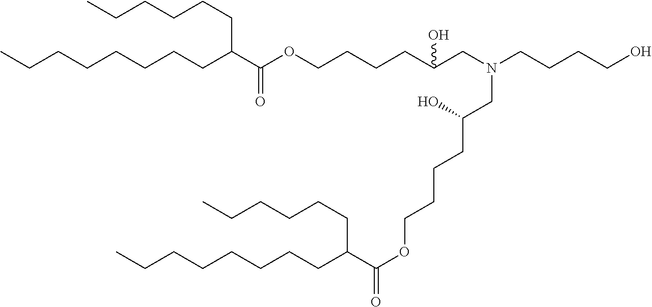 [SMILES: CCCCCCCCC(CCCCCC)C(=O)OCCCCC(O)CN(CCCCO)C[C@@H](O)CCCCOC(=O)C(CCCCCC)CCCCCCCC] |
| 106 | 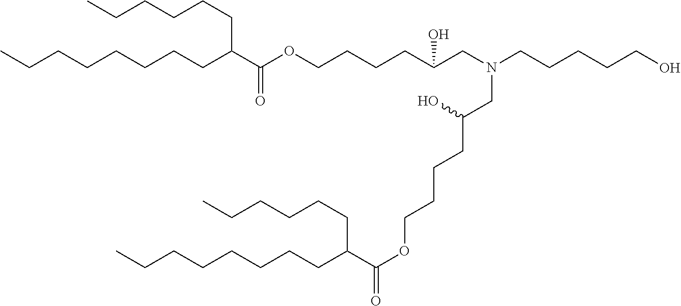 [SMILES: CCCCCCCCC(CCCCCC)C(=O)OCCCCC(O)CN(CCCCCO)C[C@H](O)CCCCOC(=O)C(CCCCCC)CCCCCCCC] |
| 107 | 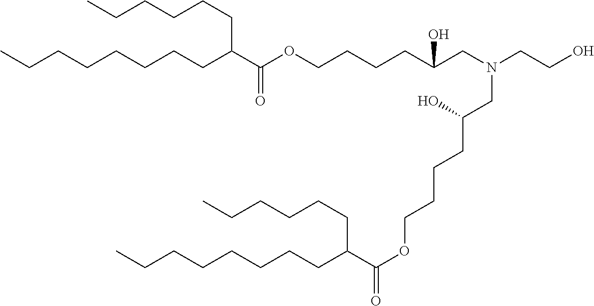 [SMILES: CCCCCCCCC(CCCCCC)C(=O)OCCCC[C@H](O)CN(CCO)C[C@@H](O)CCCCOC(=O)C(CCCCCC)CCCCCCCC] |
| 108 | 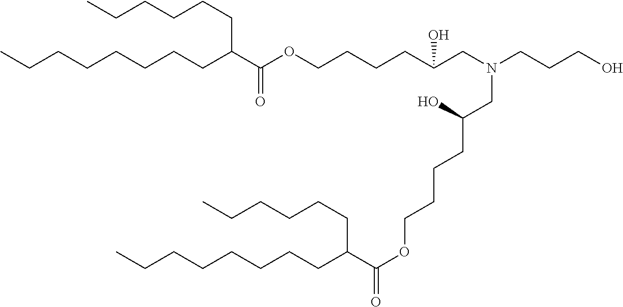 [SMILES: CCCCCCCCC(CCCCCC)C(=O)OCCCC[C@@H](O)CN(CCCO)C[C@H](O)CCCCOC(=O)C(CCCCCC)CCCCCCCC] |
| 109 | 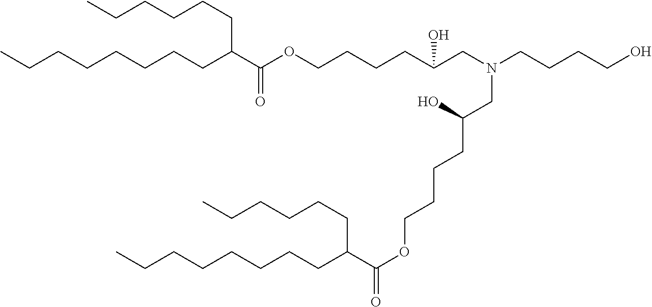 [SMILES: CCCCCCCCC(CCCCCC)C(=O)OCCCC[C@@H](O)CN(CCCCO)C[C@H](O)CCCCOC(=O)C(CCCCCC)CCCCCCCC] |
| 110 | 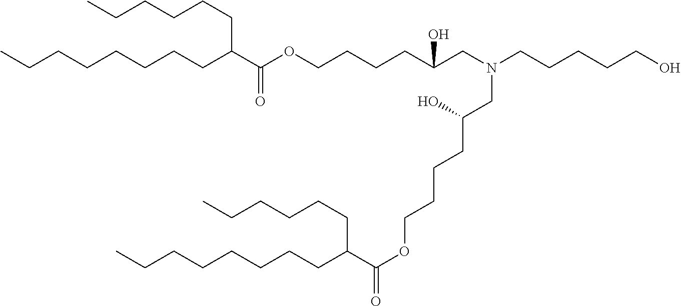 [SMILES: CCCCCCCCC(CCCCCC)C(=O)OCCCC[C@H](O)CN(CCCCCO)C[C@@H](O)CCCCOC(=O)C(CCCCCC)CCCCCCCC] |
| 111 | 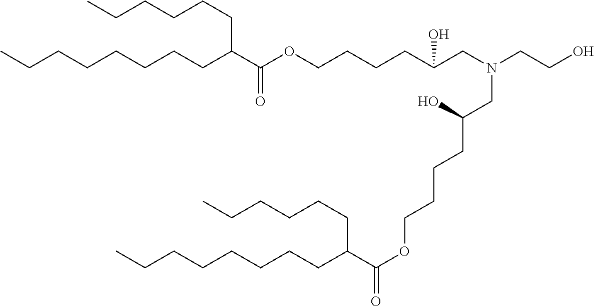 [SMILES: CCCCCCCCC(CCCCCC)C(=O)OCCCC[C@@H](O)CN(CCO)C[C@H](O)CCCCOC(=O)C(CCCCCC)CCCCCCCC] |
| 112 | 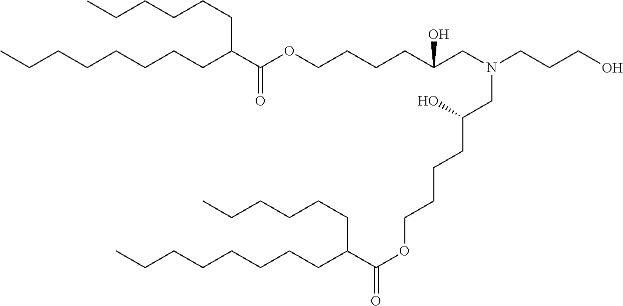 [SMILES: CCCCCCCCC(CCCCCC)C(=O)OCCCC[C@H](O)CN(CCCO)C[C@@H](O)CCCCOC(=O)C(CCCCCC)CCCCCCCC] |
| 113 | 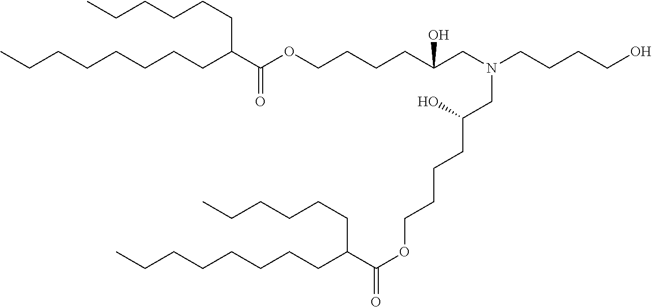 [SMILES: CCCCCCCCC(CCCCCC)C(=O)OCCCC[C@H](O)CN(CCCCO)C[C@@H](O)CCCCOC(=O)C(CCCCCC)CCCCCCCC] |
| 114 | 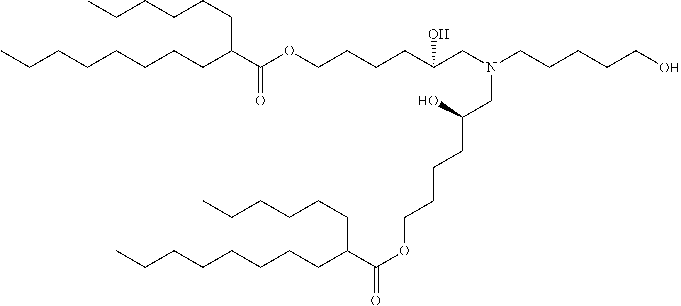 [SMILES: CCCCCCCCC(CCCCCC)C(=O)OCCCC[C@@H](O)CN(CCCCCO)C[C@H](O)CCCCOC(=O)C(CCCCCC)CCCCCCCC] |
| 115 | 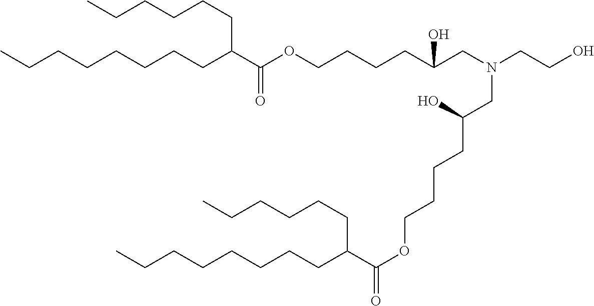 [SMILES: CCCCCCCCC(CCCCCC)C(=O)OCCCC[C@@H](O)CN(CCO)C[C@@H](O)CCCCOC(=O)C(CCCCCC)CCCCCCCC] |
| 116 | 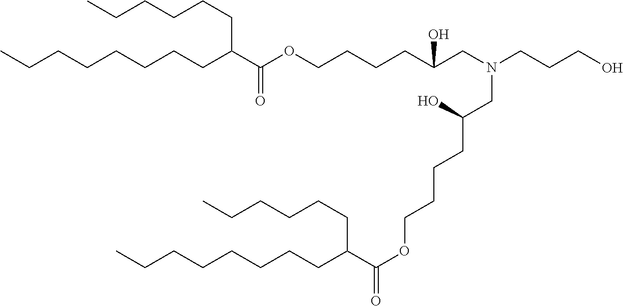 [SMILES: CCCCCCCCC(CCCCCC)C(=O)OCCCC[C@@H](O)CN(CCCO)C[C@@H](O)CCCCOC(=O)C(CCCCCC)CCCCCCCC] |
| 117 | 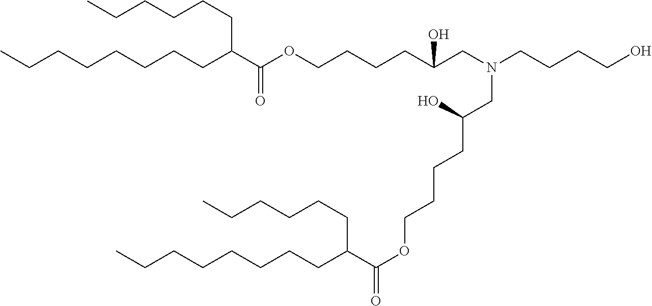 [SMILES: CCCCCCCCC(CCCCCC)C(=O)OCCCC[C@@H](O)CN(CCCCO)C[C@@H](O)CCCCOC(=O)C(CCCCCC)CCCCCCCC] |
| 118 | 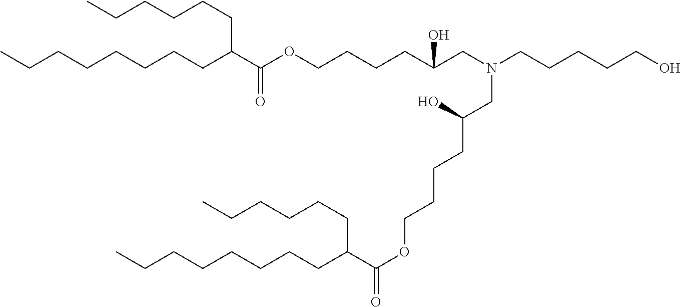 [SMILES: CCCCCCCCC(CCCCCC)C(=O)OCCCC[C@@H](O)CN(CCCCCO)C[C@@H](O)CCCCOC(=O)C(CCCCCC)CCCCCCCC] |
| 119 | 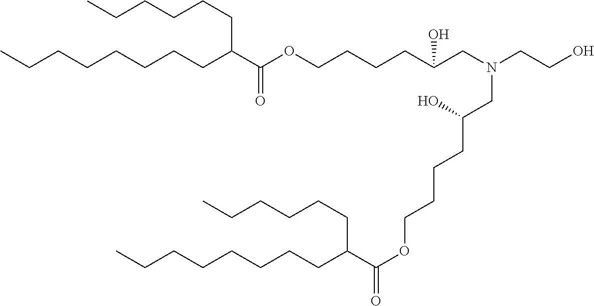 [SMILES: CCCCCCCCC(CCCCCC)C(=O)OCCCC[C@@H](O)CN(CCO)C[C@@H](O)CCCCOC(=O)C(CCCCCC)CCCCCCCC] |
| 120 | 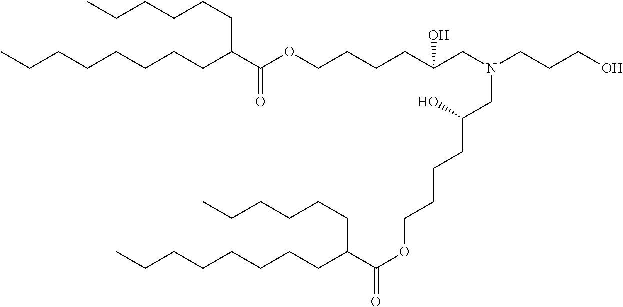 [SMILES: CCCCCCCCC(CCCCCC)C(=O)OCCCC[C@@H](O)CN(CCCO)C[C@@H](O)CCCCOC(=O)C(CCCCCC)CCCCCCCC] |
| 121 | 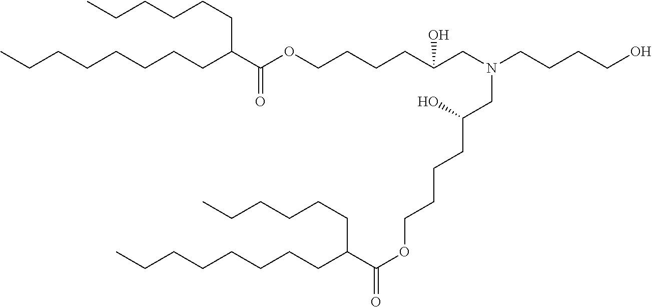 [SMILES: CCCCCCCCC(CCCCCC)C(=O)OCCCC[C@@H](O)CN(CCCCO)C[C@@H](O)CCCCOC(=O)C(CCCCCC)CCCCCCCC] |
| 122 | 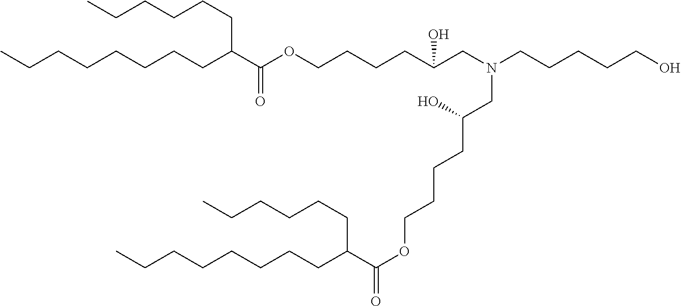 [SMILES: CCCCCCCCC(CCCCCC)C(=O)OCCCC[C@@H](O)CN(CCCCCO)C[C@@H](O)CCCCOC(=O)C(CCCCCC)CCCCCCCC] |
| 123 | 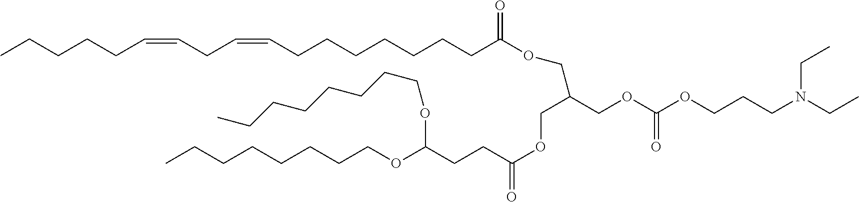 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 124 | 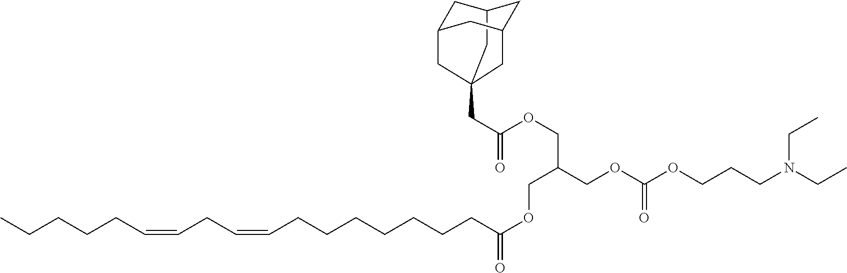 |
| 125 | 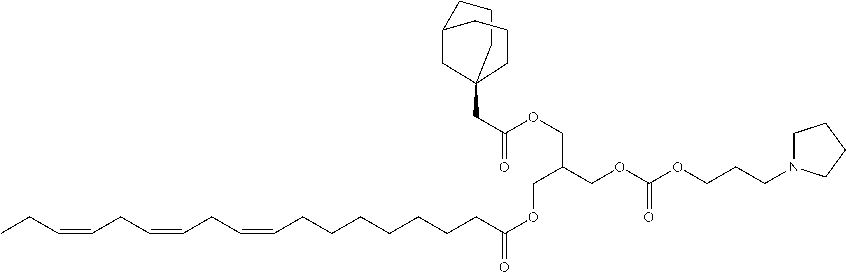 |
| 126 | 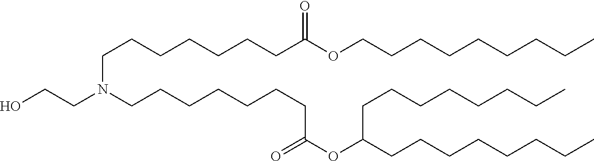 [SMILES: CCCCCCCCCOC(=O)CCCCCCCN(CCO)CCCCCCCC(=O)OC(CCCCCCCC)CCCCCCCC] |
| 127 | 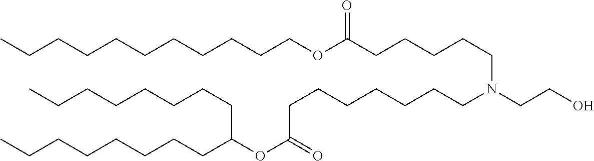 [SMILES: CCCCCCCCCCCOC(=O)CCCCCN(CCO)CCCCCCCC(=O)OC(CCCCCCCC)CCCCCCCC] |
| 128 | 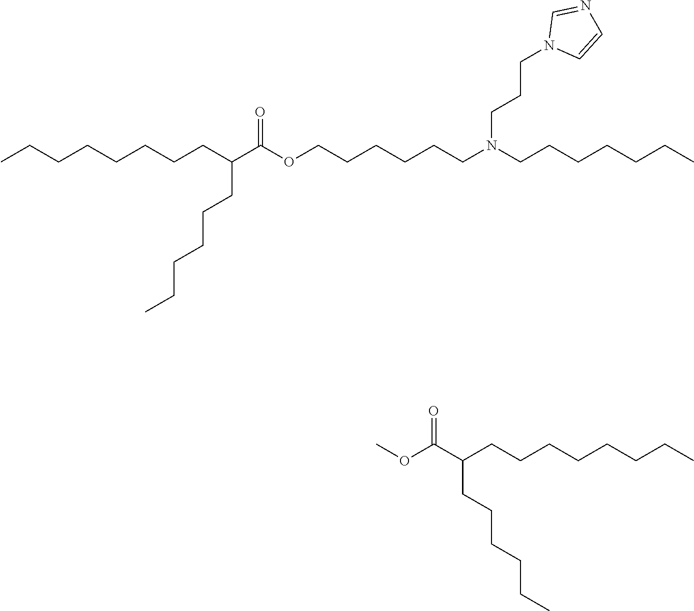 |
| 129 | 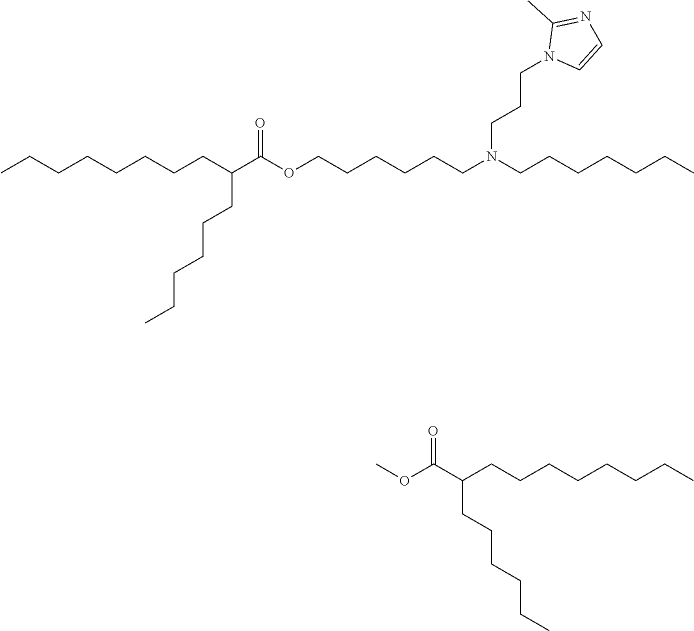 |
| 130 | 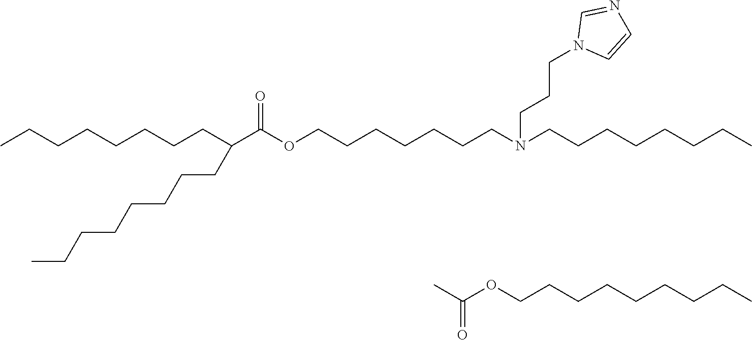 |
| 131 | 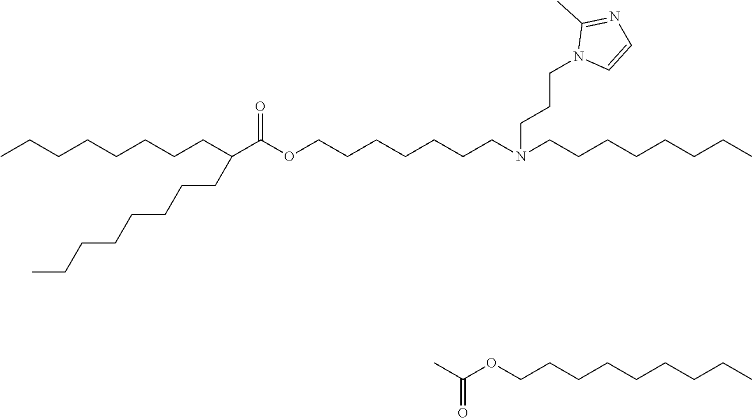 |
| 132 | 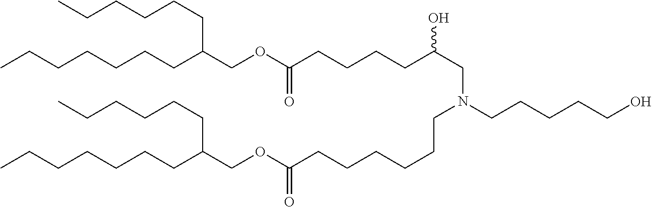 [SMILES: CCCCCCCC(CCCCCC)COC(=O)CCCCCCN(CCCCCO)CC(O)CCCCC(=O)OCC(CCCCCC)CCCCCCC] |
| 133 | 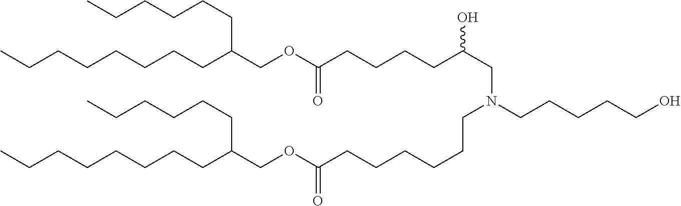 [SMILES: CCCCCCCCC(CCCCCC)COC(=O)CCCCCCN(CCCCCO)CC(O)CCCCC(=O)OCC(CCCCCC)CCCCCCCC] |
| 134 | 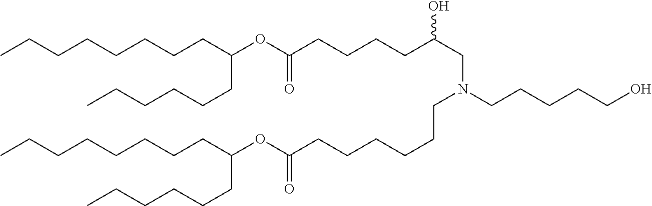 [SMILES: CCCCCCCCC(CCCCCC)OC(=O)CCCCCCN(CCCCCO)CC(O)CCCCC(=O)OC(CCCCCC)CCCCCCCC] |
| 135 | 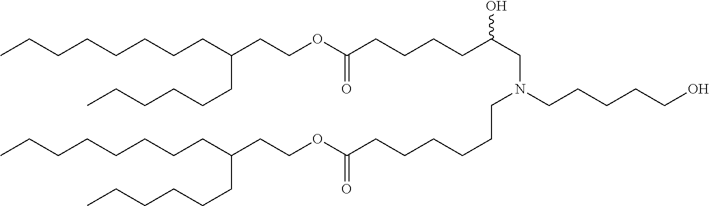 [SMILES: CCCCCCCCC(CCCCCC)CCOC(=O)CCCCCCN(CCCCCO)CC(O)CCCCC(=O)OCCC(CCCCCC)CCCCCCCC] |
| 136 | 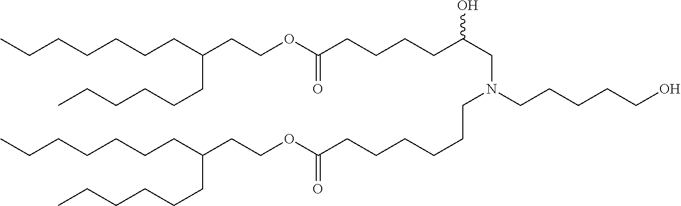 [SMILES: CCCCCCCC(CCCCCC)CCOC(=O)CCCCCCN(CCCCCO)CC(O)CCCCC(=O)OCCC(CCCCCC)CCCCCCC] |
| 137 | 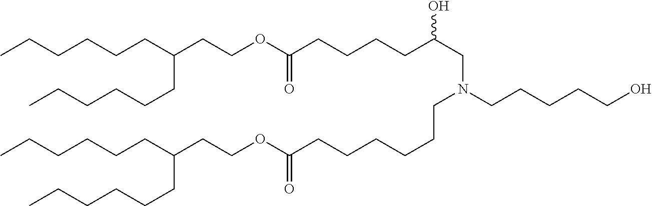 [SMILES: CCCCCCC(CCCCCC)CCOC(=O)CCCCCCN(CCCCCO)CC(O)CCCCC(=O)OCCC(CCCCCC)CCCCCC] |
| 138 | 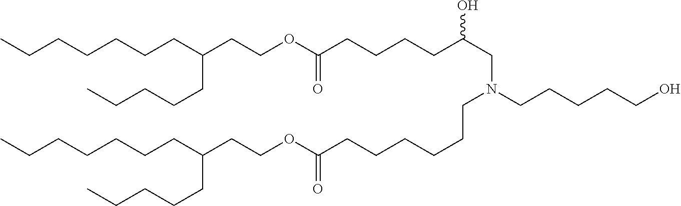 [SMILES: CCCCCCCC(CCCCC)CCOC(=O)CCCCCCN(CCCCCO)CC(O)CCCCC(=O)OCCC(CCCCC)CCCCCCC] |
| 139 | 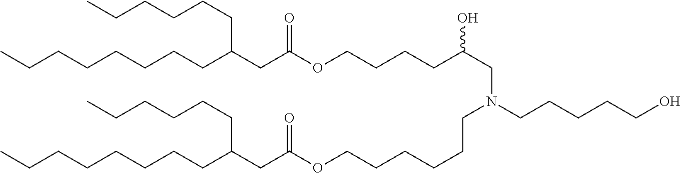 [SMILES: CCCCCCCCC(CCCCCC)CC(=O)OCCCCCCN(CCCCCO)CC(O)CCCCOC(=O)CC(CCCCCC)CCCCCCCC] |
| 140 | 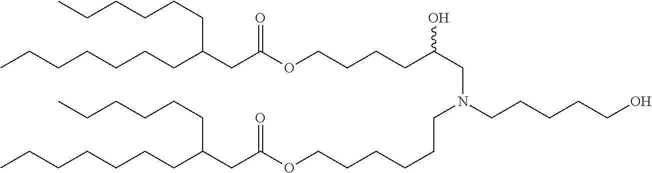 [SMILES: CCCCCCCC(CCCCCC)CC(=O)OCCCCCCN(CCCCCO)CC(O)CCCCOC(=O)CC(CCCCCC)CCCCCCC] |
| 141 | 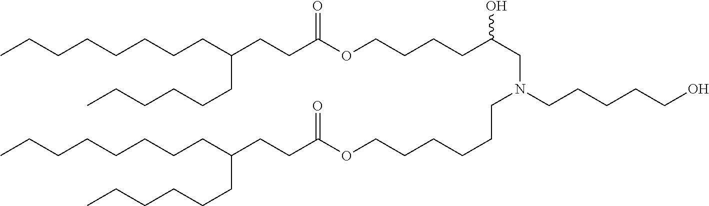 [SMILES: CCCCCCCCC(CCCCCC)CCC(=O)OCCCCCCN(CCCCCO)CC(O)CCCCOC(=O)CCC(CCCCCC)CCCCCCCC] |
| 142 | 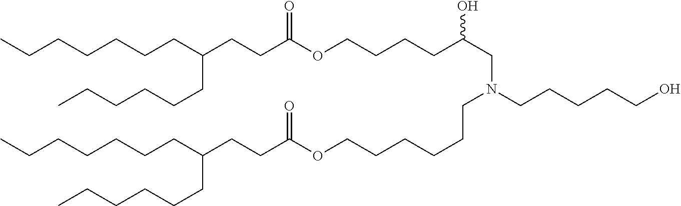 [SMILES: CCCCCCCC(CCCCCC)CCC(=O)OCCCCCCN(CCCCCO)CC(O)CCCCOC(=O)CCC(CCCCCC)CCCCCCC] |
| 143 | 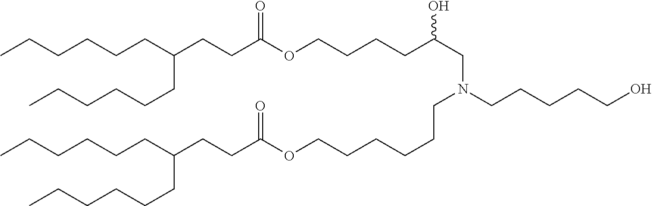 [SMILES: CCCCCCC(CCCCCC)CCC(=O)OCCCCCCN(CCCCCO)CC(O)CCCCOC(=O)CCC(CCCCCC)CCCCCC] |
| 144 | 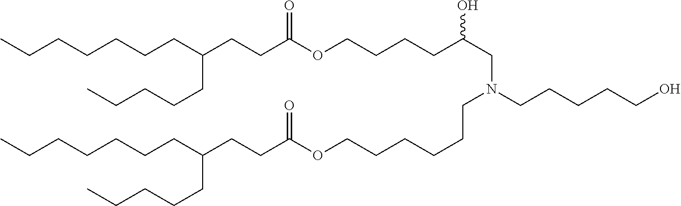 [SMILES: CCCCCCCC(CCCCC)CCC(=O)OCCCCCCN(CCCCCO)CC(O)CCCCOC(=O)CCC(CCCCC)CCCCCCC] |
| 145 | 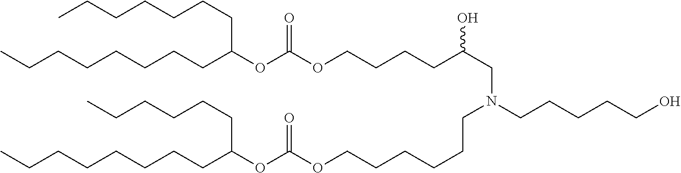 [SMILES: CCCCCCCCC(CCCCCC)OC(=O)OCCCCCCN(CCCCCO)CC(O)CCCCOC(=O)OC(CCCCCCC)CCCCCCCC] |
| 146 | 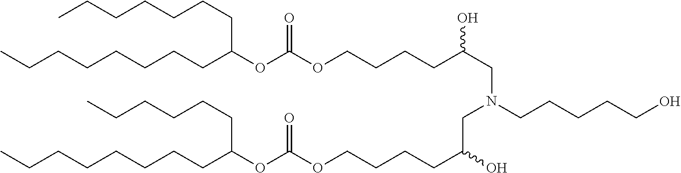 [SMILES: CCCCCCCCC(CCCCCC)OC(=O)OCCCCC(O)CN(CCCCCO)CC(O)CCCCOC(=O)OC(CCCCCCC)CCCCCCCC] |
| 147 | 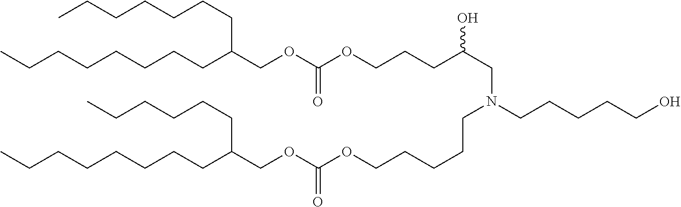 [SMILES: CCCCCCCCC(CCCCCC)COC(=O)OCCCCCN(CCCCCO)CC(O)CCCOC(=O)OCC(CCCCCCC)CCCCCCCC] |
| 148 | 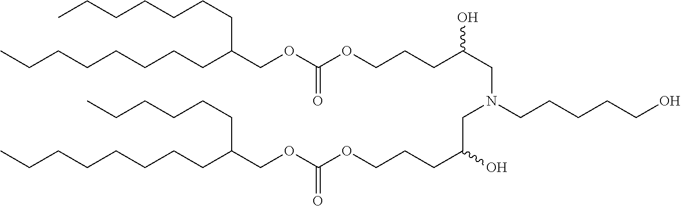 [SMILES: CCCCCCCCC(CCCCCC)COC(=O)OCCCC(O)CN(CCCCCO)CC(O)CCCOC(=O)OCC(CCCCCCC)CCCCCCCC] |
| 149 | 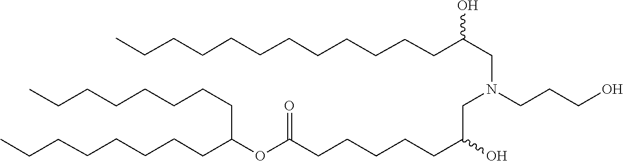 [SMILES: CCCCCCCCCCCCC(O)CN(CCCO)CC(O)CCCCCC(=O)OC(CCCCCCCC)CCCCCCCC] |
| 150 | 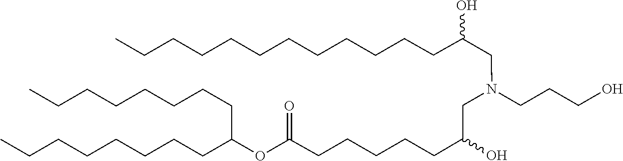 [SMILES: CCCCCCCCCCCCC(O)CN(CCCO)CC(O)CCCCCC(=O)OC(CCCCCCCC)CCCCCCCC] |
| 151 | 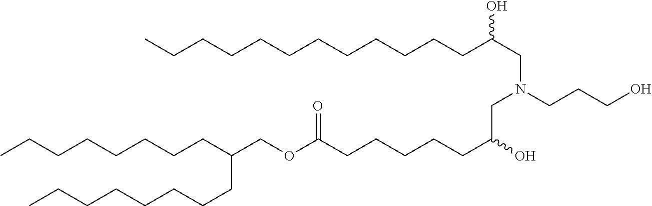 [SMILES: CCCCCCCCCCCCC(O)CN(CCCO)CC(O)CCCCCC(=O)OCC(CCCCCCCC)CCCCCCCC] |
| 152 | 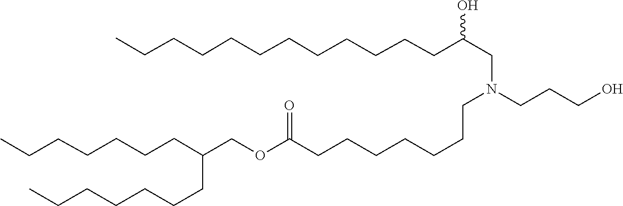 [SMILES: CCCCCCCCCCCCC(O)CN(CCCO)CCCCCCCC(=O)OCC(CCCCCCC)CCCCCCC] |
| 153 | 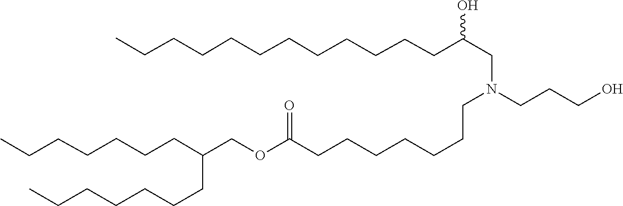 [SMILES: CCCCCCCCCCCCC(O)CN(CCCO)CCCCCCCC(=O)OCC(CCCCCCC)CCCCCCC] |
| 154 | 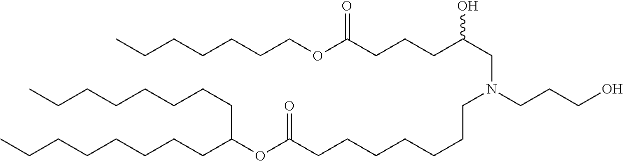 [SMILES: CCCCCCCCC(CCCCCCCC)OC(=O)CCCCCCCN(CCCO)CC(O)CCCC(=O)OCCCCCCC] |
| 155 | 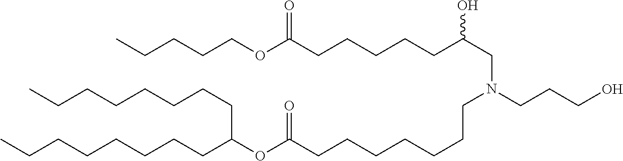 [SMILES: CCCCCCCCC(CCCCCCCC)OC(=O)CCCCCCCN(CCCO)CC(O)CCCCCC(=O)OCCCCC] |
| 156 | 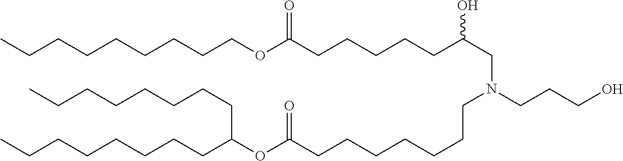 [SMILES: CCCCCCCCCOC(=O)CCCCCC(O)CN(CCCO)CCCCCCCC(=O)OC(CCCCCCCC)CCCCCCCC] |
| 157 | 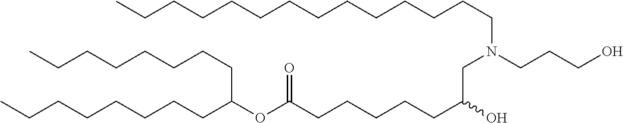 [SMILES: CCCCCCCCCCCCCCN(CCCO)CC(O)CCCCCC(=O)OC(CCCCCCCC)CCCCCCCC] |
| 158 | 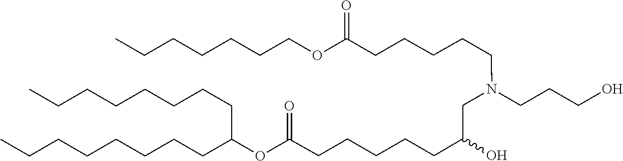 [SMILES: CCCCCCCCC(CCCCCCCC)OC(=O)CCCCCC(O)CN(CCCO)CCCCCC(=O)OCCCCCCC] |
| 159 | 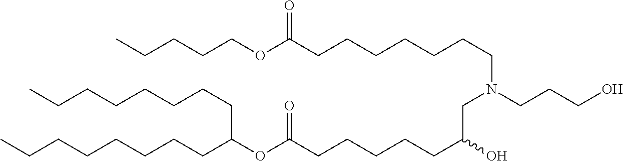 [SMILES: CCCCCCCCC(CCCCCCCC)OC(=O)CCCCCC(O)CN(CCCO)CCCCCCCC(=O)OCCCCC] |
| 160 | 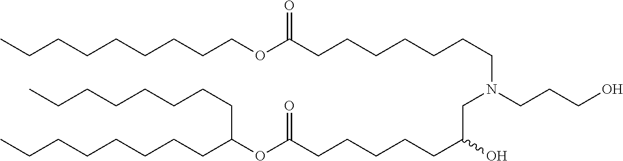 [SMILES: CCCCCCCCCOC(=O)CCCCCCCN(CCCO)CC(O)CCCCCC(=O)OC(CCCCCCCC)CCCCCCCC] |
| 161 | 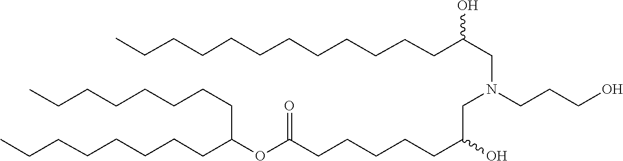 [SMILES: CCCCCCCCCCCCC(O)CN(CCCO)CC(O)CCCCCC(=O)OC(CCCCCCCC)CCCCCCCC] |
| 162 | 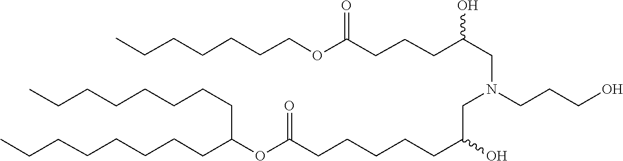 [SMILES: CCCCCCCCC(CCCCCCCC)OC(=O)CCCCCC(O)CN(CCCO)CC(O)CCCC(=O)OCCCCCCC] |
| 163 | 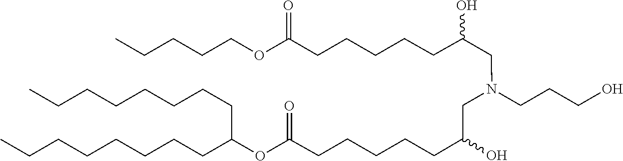 [SMILES: CCCCCCCCC(CCCCCCCC)OC(=O)CCCCCC(O)CN(CCCO)CC(O)CCCCCC(=O)OCCCCC] |
| 164 | 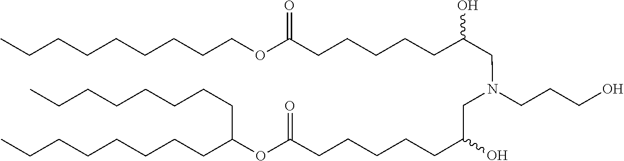 [SMILES: CCCCCCCCCOC(=O)CCCCCC(O)CN(CCCO)CC(O)CCCCCC(=O)OC(CCCCCCCC)CCCCCCCC] |
| 165 | 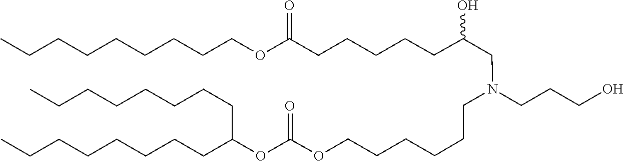 [SMILES: CCCCCCCCCOC(=O)CCCCCC(O)CN(CCCO)CCCCCCOC(=O)OC(CCCCCCCC)CCCCCCCC] |
| 166 | 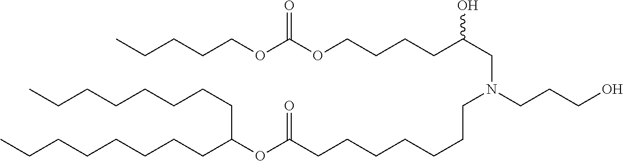 [SMILES: CCCCCCCCC(CCCCCCCC)OC(=O)CCCCCCCN(CCCO)CC(O)CCCCOC(=O)OCCCCC] |
| 167 | 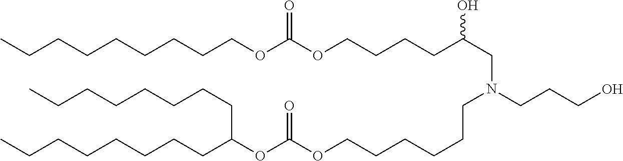 [SMILES: CCCCCCCCCOC(=O)OCCCCC(O)CN(CCCO)CCCCCCOC(=O)OC(CCCCCCCC)CCCCCCCC] |
| 168 | 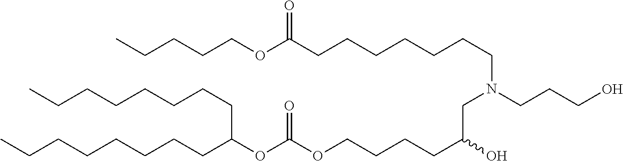 [SMILES: CCCCCCCCC(CCCCCCCC)OC(=O)OCCCCC(O)CN(CCCO)CCCCCCCC(=O)OCCCCC] |
| 169 | 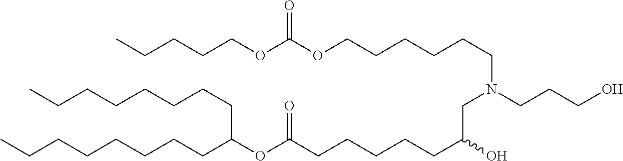 [SMILES: CCCCCCCCC(CCCCCCCC)OC(=O)CCCCCC(O)CN(CCCO)CCCCCCOC(=O)OCCCCC] |
| 170 | 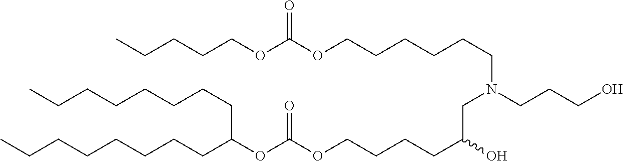 [SMILES: CCCCCCCCC(CCCCCCCC)OC(=O)OCCCCC(O)CN(CCCO)CCCCCCOC(=O)OCCCCC] |
| 171 | 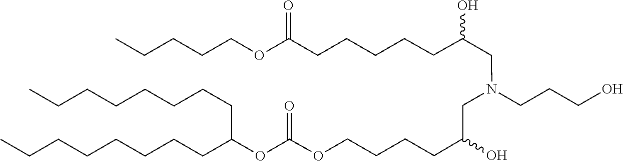 [SMILES: CCCCCCCCC(CCCCCCCC)OC(=O)OCCCCC(O)CN(CCCO)CC(O)CCCCCC(=O)OCCCCC] |
| 172 | 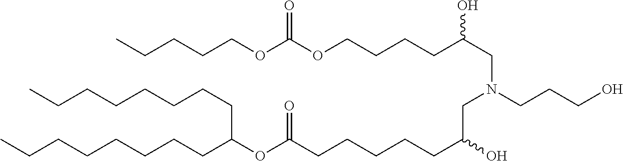 [SMILES: CCCCCCCCC(CCCCCCCC)OC(=O)CCCCCC(O)CN(CCCO)CC(O)CCCCOC(=O)OCCCCC] |
| 173 | 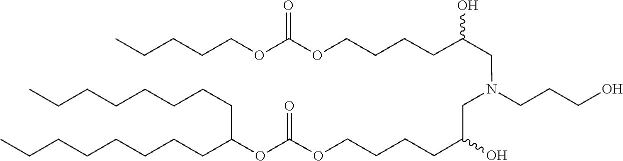 [SMILES: CCCCCCCCC(CCCCCCCC)OC(=O)OCCCCC(O)CN(CCCO)CC(O)CCCCOC(=O)OCCCCC] |
| 174 | 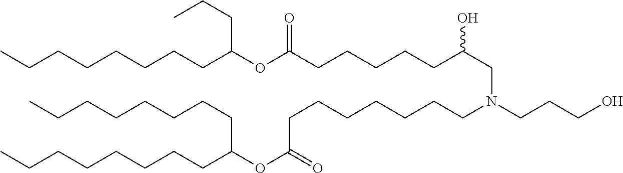 [SMILES: CCCCCCCCC(CCC)OC(=O)CCCCCC(O)CN(CCCO)CCCCCCCC(=O)OC(CCCCCCCC)CCCCCCCC] |
| 175 | 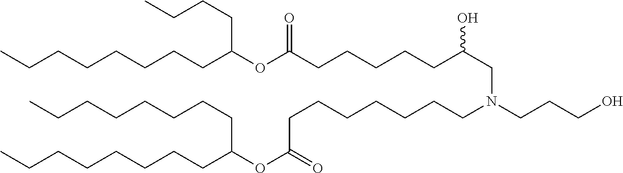 [SMILES: CCCCCCCCC(CCCC)OC(=O)CCCCCC(O)CN(CCCO)CCCCCCCC(=O)OC(CCCCCCCC)CCCCCCCC] |
| 176 | 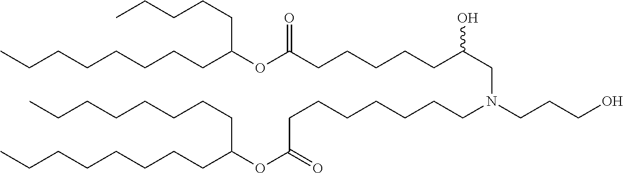 [SMILES: CCCCCCCCC(CCCCC)OC(=O)CCCCCC(O)CN(CCCO)CCCCCCCC(=O)OC(CCCCCCCC)CCCCCCCC] |
| 177 | 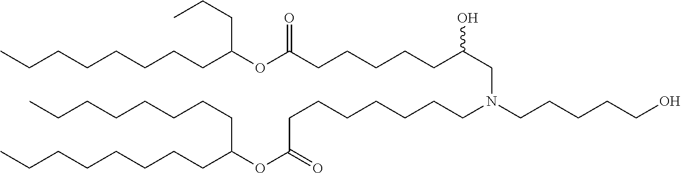 [SMILES: CCCCCCCCC(CCC)OC(=O)CCCCCC(O)CN(CCCCCO)CCCCCCCC(=O)OC(CCCCCCCC)CCCCCCCC] |
| 178 | 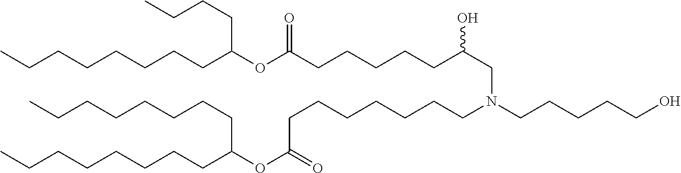 [SMILES: CCCCCCCCC(CCCC)OC(=O)CCCCCC(O)CN(CCCCCO)CCCCCCCC(=O)OC(CCCCCCCC)CCCCCCCC] |
| 179 | 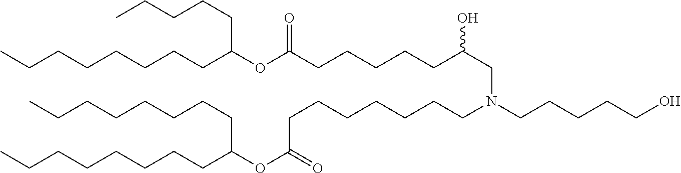 [SMILES: CCCCCCCCC(CCCCC)OC(=O)CCCCCC(O)CN(CCCCCO)CCCCCCCC(=O)OC(CCCCCCCC)CCCCCCCC] |
| 180 | 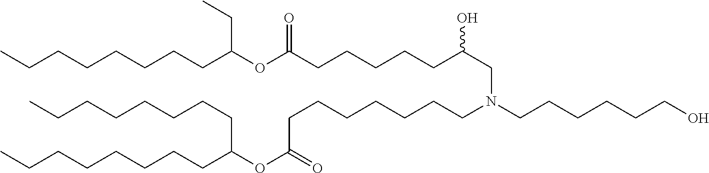 [SMILES: CCCCCCCCC(CC)OC(=O)CCCCCC(O)CN(CCCCCCO)CCCCCCCC(=O)OC(CCCCCCCC)CCCCCCCC] |
| 181 | 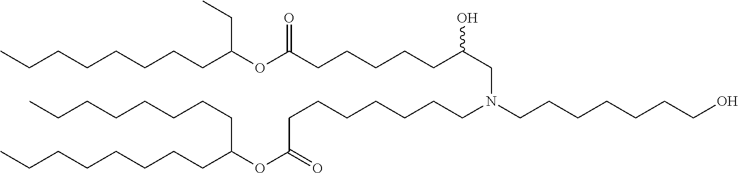 [SMILES: CCCCCCCCC(CC)OC(=O)CCCCCC(O)CN(CCCCCCCO)CCCCCCCC(=O)OC(CCCCCCCC)CCCCCCCC] |
| 182 | 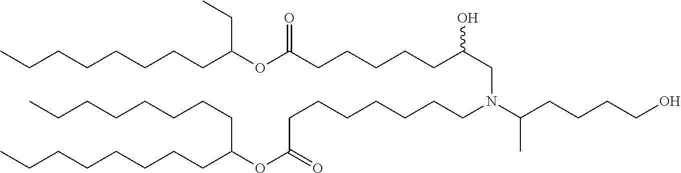 [SMILES: CCCCCCCCC(CC)OC(=O)CCCCCC(O)CN(CCCCCCCC(=O)OC(CCCCCCCC)CCCCCCCC)C(C)CCCCO] |
| 183 | 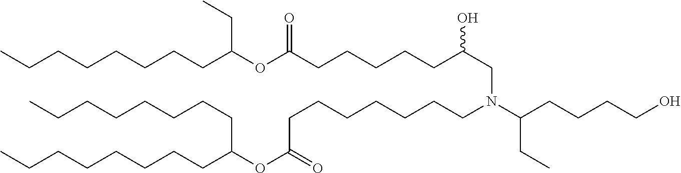 [SMILES: CCCCCCCCC(CC)OC(=O)CCCCCC(O)CN(CCCCCCCC(=O)OC(CCCCCCCC)CCCCCCCC)C(CC)CCCCO] |
| 184 | 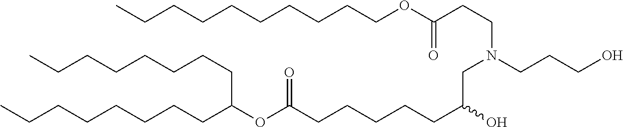 [SMILES: CCCCCCCCCCOC(=O)CCN(CCCO)CC(O)CCCCCC(=O)OC(CCCCCCCC)CCCCCCCC] |
| 185 | 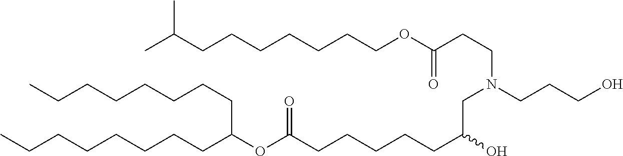 [SMILES: CCCCCCCCC(CCCCCCCC)OC(=O)CCCCCC(O)CN(CCCO)CCC(=O)OCCCCCCCC(C)C] |

In some embodiments, an ionizable lipid is a compound of Formula (15):

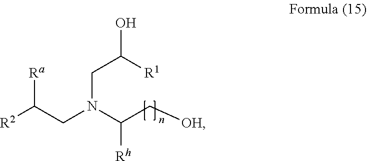

or is a pharmaceutically acceptable salt thereof, wherein:
   n* is an integer from 1 to 7;
   R^{a} is hydrogen or hydroxyl;
   R^{h} is hydrogen or C₁-C₆ alkyl;
   R¹ is C₁-C₃₀ alkyl or R¹*
   R² is C₁-C₃₀ alkyl or R²*,
   R¹* and R²* are independently selected from:
      —(CH₂)_{q}C(O)O(CH₂)ᵣC(R⁸)(R⁹)(R¹⁰),
      —(CH₂)_{q}OC(O)(CH₂)ᵣC(R⁸)(R⁹)(R¹⁰), and
      —(CH₂)_{q}OC(O)O(CH₂)ᵣC(R⁸)(R⁹)(R¹⁰);
   wherein:
      q is an integer from 0 to 12,
      r is an integer from 0 to 6, wherein at least one occurrence of r is not 0;
      R⁸ is H or R¹¹;
      R⁹, R¹⁰, and R¹¹ are each independently C₁-C₂₀ alkyl or C₂-C₂₀-alkenyl; and
   wherein (i) R¹ is R¹*, (ii) R² is R²*, or (iii) R¹ is R¹* and R² is R²*.

In some embodiments of Formula (15), Rₐ is hydrogen and the ionizable lipid is of Formula (16):

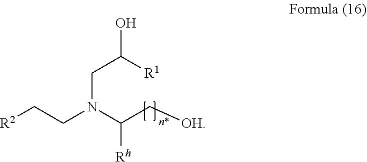

or is a pharmaceutically acceptable salt thereof, wherein:
n* is an integer from 1 to 7.

In some embodiments of Formula (16), the ionizable lipid is of Formula (17):

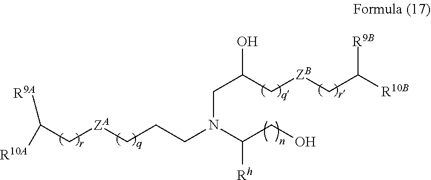

or a pharmaceutically acceptable salt thereof, wherein:
   n is an integer from 1 to 7;
   q and q′ are each independently integers from 0 to 12;
   r and r′ are each independently integers from 0 to 6, wherein at least one of r or r′ is not 0;
   Z^{A} and Z^{B} are each independently selected from ∧—C(O)O—, ∧—OC(O), and —OC(O)O—; where ∧denotes the attachment point to —(CH₂)_{q}— or —(CH₂)_{q}′-; and
   R^{9A}, R^{9B}, R^{10A}, and R^{10B} are each independently C₁-C₂₀ alkyl or C₂-C₂₀ alkenyl.

In some embodiments of Formula (17), Z^{A} and Z^{B} are ∧—C(O)O—, and the ionizable lipid is of Formula (17a-1)

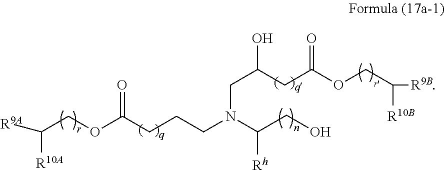

In some embodiments of Formula (17), Z^{A} and Z^{B} are ∧—OC(O)—, and the ionizable lipid is of Formula (17a-2)

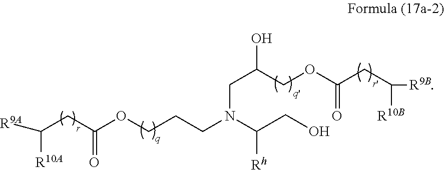

In some embodiments of Formula (17), Z^{A} and Z^{B} are —O(C)(O)O—, and the ionizable lipid is represented by Formula (17a-3):

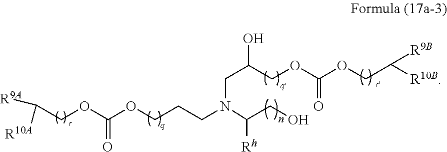

In some embodiments of Formula (15), R^{a} is hydroxyl and the ionizable lipid is of Formula (18):

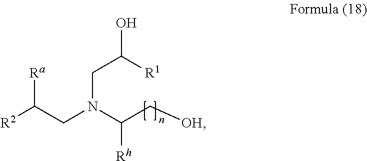

or is a pharmaceutically acceptable salt thereof, wherein:
   n* is an integer from 1 to 7;
   R^{h} is hydrogen or C₁-C₆ alkyl;
   R¹ is C₁-C₃₀ alkyl or R¹*
   R² is C₁-C₃₀ alkyl or R²*,
   R¹* and R²* are independently selected from:
   —(CH₂)_{q}C(O)O(CH₂)ᵣC(R⁸)(R⁹)(R¹⁰),
   —(CH₂)_{q}OC(O)(CH₂)ᵣC(R⁸)(R⁹)(R¹⁰), and
   —(CH₂)_{q}OC(O)O(CH₂)ᵣC(R⁸)(R⁹)(R¹⁰);
wherein:
   q is an integer from 0 to 12,
   r is an integer from 0 to 6, wherein at least one occurrence of r is not 0;
   R⁸ is hydrogen or R¹¹;
   R⁹, R¹⁰, and R¹¹ are each independently C₁-C₂₀ alkyl or C₂-C₂₀-alkenyl;
wherein (i) R¹ is R¹*, (ii) R² is R²*, or (iii) R¹ is R¹* and R² is R²*, and
wherein, for (iii), (a) R¹* and R²* are different or (b) R⁹ and R¹⁰ have different numbers of carbon atoms for at least one of R¹* and R²*.

In some embodiments of Formula (18), the ionizable lipid of is of Formula (19):

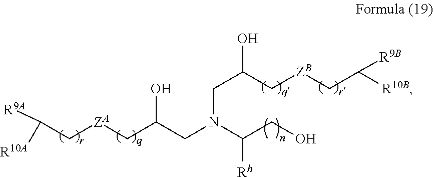

or is a pharmaceutically acceptable salt thereof, wherein:
   n is an integer from 1 to 7;
   q and q′ are each independently integers from 0 to 12;
   r and r′ are each independently integers from 0 to 6, wherein at least one of r or r′ is not 0;
   Z^{A} and Z^{B} are each independently selected from ∧—C(O)O—, ∧—OC(O), and —OC(O)O—;
   where ∧ denotes the attachment point to —(CH₂)_{q}— or —(CH₂)_{q}′; and
   R^{9A}, R^{9B}, R^{10A} and R^{10B} are each independently C₁-C₂₀ alkyl or C₂-C₂₀ alkenyl.

In some embodiments of Formula (19), Z^{A} and Z^{B} are ∧—C(O)O—, and the ionizable lipid is of Formula (19a-1):

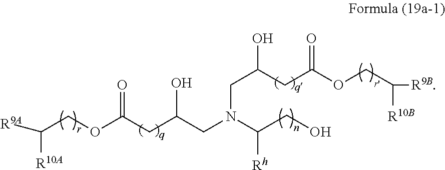

In some embodiments of Formula (19), Z^{A} and Z^{B} are ∧—OC(O)—, and the ionizable lipid is of Formula (19a-2):

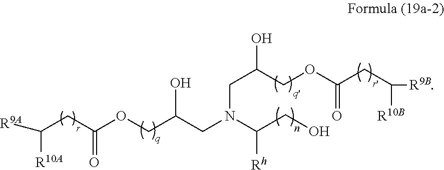

In some embodiments of Formula (19), Z^{A} and Z^{B} are —O (C) (0)O—, and the ionizable lipid is represented by Formula (19a-3):

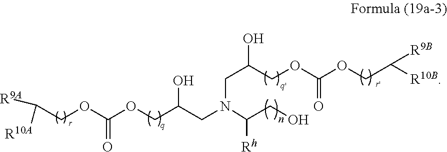

In some embodiments of Formula (15), R′ is C1-C30 alkyl, and the ionizable lipid is of Formula (20):

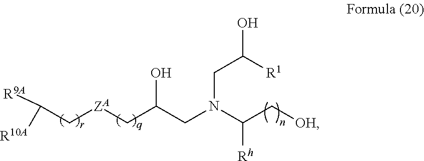

or is a pharmaceutically acceptable salt thereof, wherein:
   Z^{A} is selected from ∧—C(O)O—, ∧—OC(O)—, and —OC(O)O—; where ∧ denotes the attachment point to —(CH₂)_{q}—;
   R^{9A} and R^{10A} are each independently C₁-C₂₀ alkyl or C₂-C₂₀ alkenyl;
   n is an integer from 1 to 7;
   q is an integer from 0 to 12; and
   r is an integer from 1 to 6.

In some embodiments of Formula (20), Z^{A} is ∧—C(O)O—, and the ionizable lipid is of Formula (20a-1):

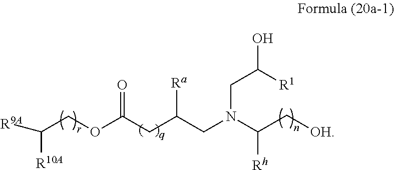

In some embodiments of Formula (20), Z^{A} is ∧—OC(O)—, and the ionizable lipid is of Formula (20a-2):

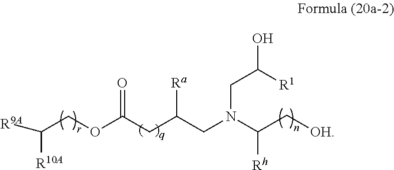

In some embodiments of Formula (20), Z^{A} is —OC(O)O—, and the ionizable lipid is of Formula (20a-3):

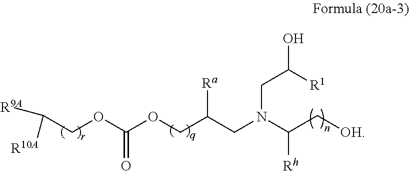

In some embodiments of Formula (15), R2 is C₁-C30 alkyl, and the ionizable lipid is of Formula (21):

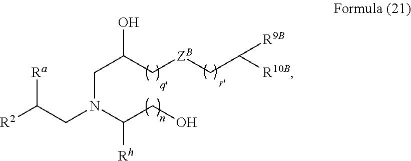

or is a pharmaceutically acceptable salt thereof, wherein:
   Z^{B} is selected from ∧—C(O)O—, ∧—OC(O)—, and —OC(O)O—; where ∧ denotes the attachment point to —(CH₂) q′ ˜;
R^{9B} and R^{10B} are each independently C₁-C₂₀ alkyl or C₂-C₂₀ alkenyl;
   n is an integer from 1 to 7;
   q′ is an integer from 0 to 12; and
   r′ is an integer from 1 to 6.

In some embodiments of Formula (21), Z^{B} is ∧—C(O)O—, and the ionizable lipid is of Formula (21a-1):

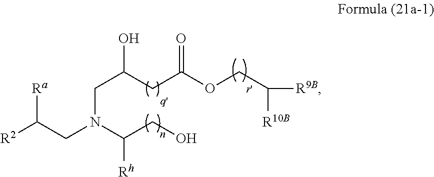

In some embodiments of Formula (21), Z^{B} is ∧—OC(O)—, and the ionizable lipid is of Formula (21a-2):

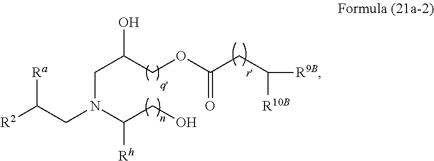

In some embodiments of Formula (21), Z^{B} is —OC(O)O—, and the ionizable lipid is of Formula (21a-3):

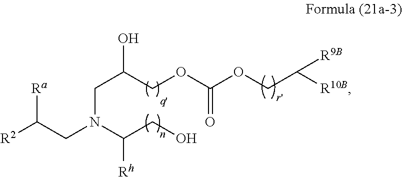

In some embodiments, an ionizable lipid is selected from the table below:

| Ionizable | |
|---|---|
| lipid | |
| number | Structure |
| 1-a | 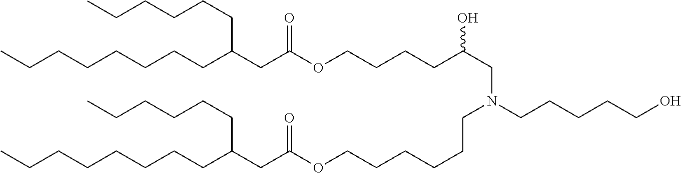 [SMILES: CCCCCCCCC(CCCCCC)CC(=O)OCCCCCCN(CCCCCO)CC(O)CCCCOC(=O)CC(CCCCCC)CCCCCCCC] |
| 1-b | 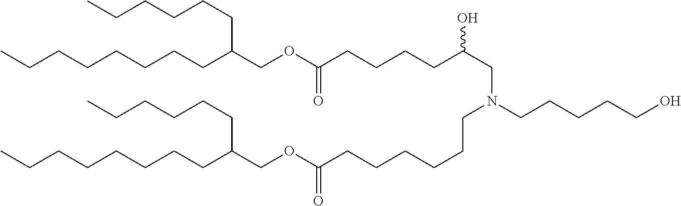 [SMILES: CCCCCCCCC(CCCCCC)COC(=O)CCCCCCN(CCCCCO)CC(O)CCCCC(=O)OCC(CCCCCC)CCCCCCCC] |
| 1-c | 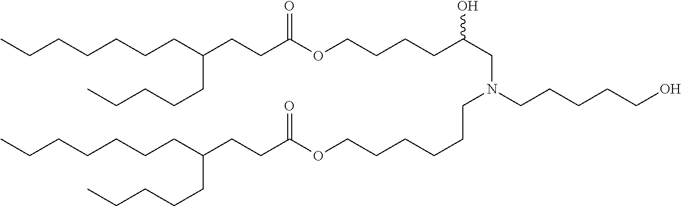 [SMILES: CCCCCCCC(CCCCC)CCC(=O)OCCCCCCN(CCCCCO)CC(O)CCCCOC(=O)CCC(CCCCC)CCCCCCC] |
| 1-d | 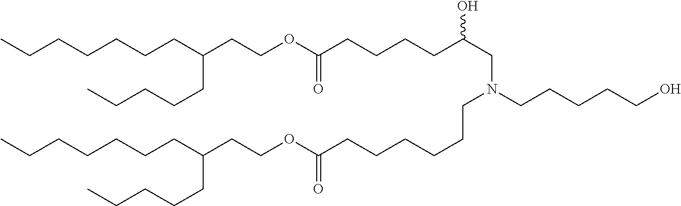 [SMILES: CCCCCCCC(CCCCC)CCOC(=O)CCCCCCN(CCCCCO)CC(O)CCCCC(=O)OCCC(CCCCC)CCCCCCC] |
| 1-e | 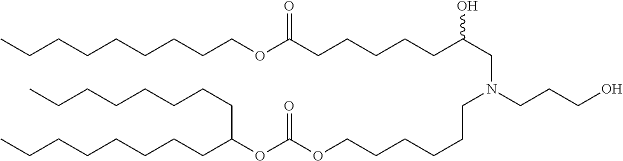 [SMILES: CCCCCCCCCOC(=O)CCCCCC(O)CN(CCCO)CCCCCCOC(=O)OC(CCCCCCCC)CCCCCCCC] |
| 1-f | 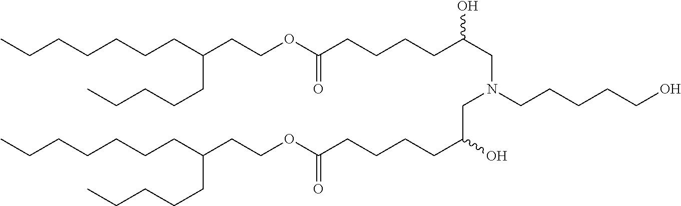 [SMILES: CCCCCCCC(CCCCC)CCOC(=O)CCCCC(O)CN(CCCCCO)CC(O)CCCCC(=O)OCCC(CCCCC)CCCCCCC] |
| 1-g | 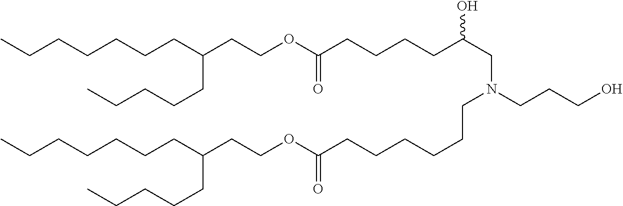 [SMILES: CCCCCCCC(CCCCC)CCOC(=O)CCCCCCN(CCCO)CC(O)CCCCC(=O)OCCC(CCCCC)CCCCCCC] |
| 1-h | 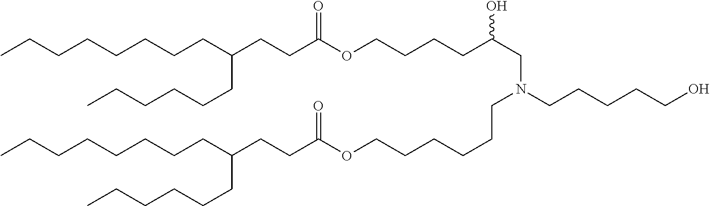 [SMILES: CCCCCCCCC(CCCCCC)CCC(=O)OCCCCCCN(CCCCCO)CC(O)CCCCOC(=O)CCC(CCCCCC)CCCCCCCC] |
| 1-i | 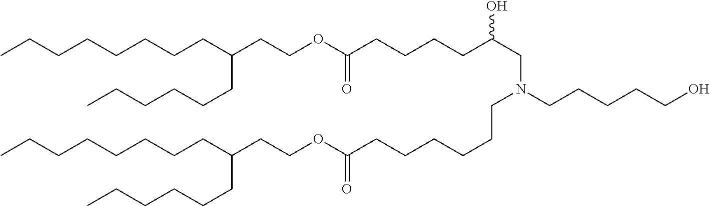 [SMILES: CCCCCCCCC(CCCCCC)CCOC(=O)CCCCCCN(CCCCCO)CC(O)CCCCC(=O)OCCC(CCCCCC)CCCCCCCC] |
| 1-j | 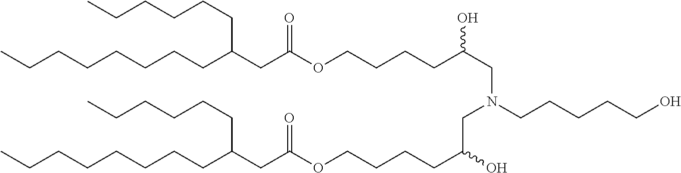 [SMILES: CCCCCCCCC(CCCCCC)CC(=O)OCCCCC(O)CN(CCCCCO)CC(O)CCCCOC(=O)CC(CCCCCC)CCCCCCCC] |
| 1-k | 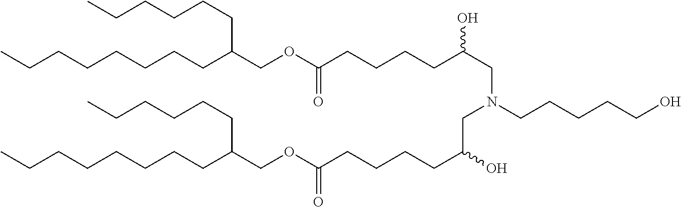 [SMILES: CCCCCCCCC(CCCCCC)COC(=O)CCCCC(O)CN(CCCCCO)CC(O)CCCCC(=O)OCC(CCCCCC)CCCCCCCC] |
| 1-l | 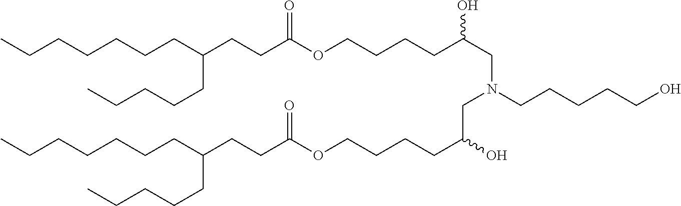 [SMILES: CCCCCCCC(CCCCC)CCC(=O)OCCCCC(O)CN(CCCCCO)CC(O)CCCCOC(=O)CCC(CCCCC)CCCCCCC] |
| 1-m | 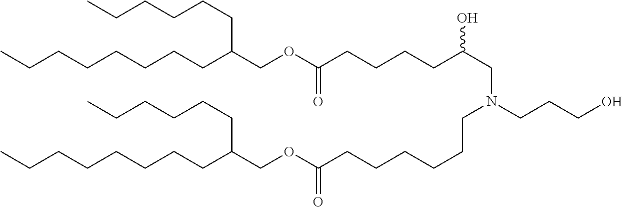 [SMILES: CCCCCCCCC(CCCCCC)COC(=O)CCCCCCN(CCCO)CC(O)CCCCC(=O)OCC(CCCCCC)CCCCCCCC] |
| 1-m | 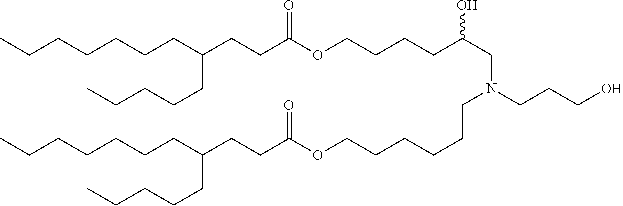 [SMILES: CCCCCCCC(CCCCC)CCC(=O)OCCCCCCN(CCCO)CC(O)CCCCOC(=O)CCC(CCCCC)CCCCCCC] |
| 1-n | 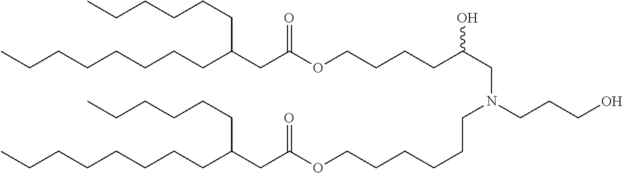 [SMILES: CCCCCCCCC(CCCCCC)CC(=O)OCCCCCCN(CCCO)CC(O)CCCCOC(=O)CC(CCCCCC)CCCCCCCC] |
| 1-o | 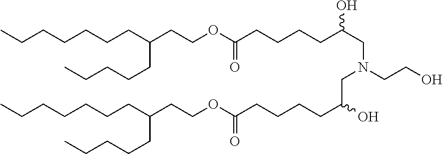 [SMILES: CCCCCCCC(CCCCC)CCOC(=O)CCCCC(O)CN(CCO)CC(O)CCCCC(=O)OCCC(CCCCC)CCCCCCC] |
| 1-p | 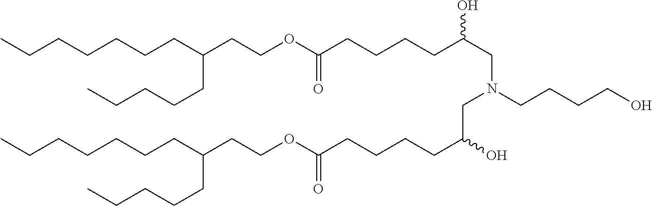 [SMILES: CCCCCCCC(CCCCC)CCOC(=O)CCCCC(O)CN(CCCCO)CC(O)CCCCC(=O)OCCC(CCCCC)CCCCCCC] |
| 1-q | 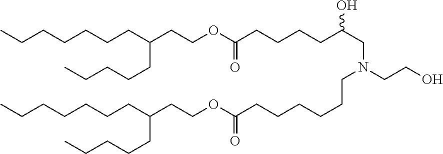 [SMILES: CCCCCCCC(CCCCC)CCOC(=O)CCCCCCN(CCO)CC(O)CCCCC(=O)OCCC(CCCCC)CCCCCCC] |
| 1-r | 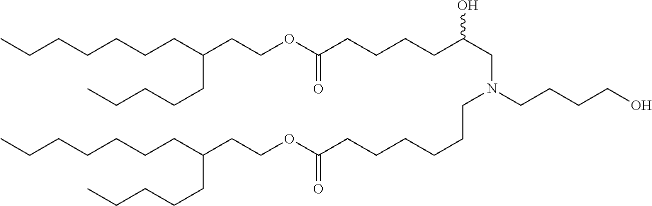 [SMILES: CCCCCCCC(CCCCC)CCOC(=O)CCCCCCN(CCCCO)CC(O)CCCCC(=O)OCCC(CCCCC)CCCCCCC] |
| 1-s | 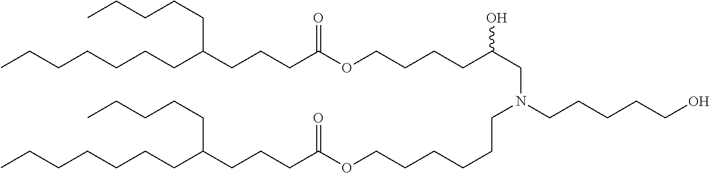 [SMILES: CCCCCCCC(CCCCC)CCCC(=O)OCCCCCCN(CCCCCO)CC(O)CCCCOC(=O)CCCC(CCCCC)CCCCCCC] |
| 1-t | 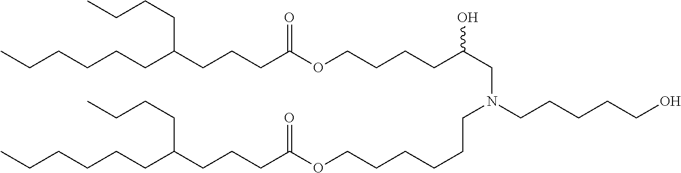 [SMILES: CCCCCCC(CCCC)CCCC(=O)OCCCCCCN(CCCCCO)CC(O)CCCCOC(=O)CCCC(CCCC)CCCCCC] |
| 1-u | 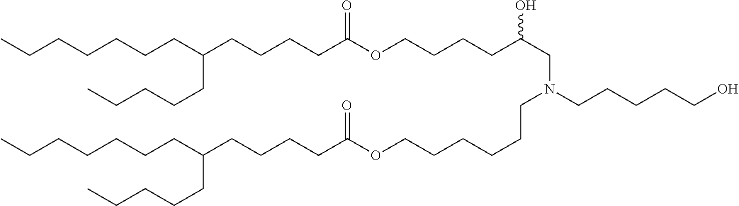 [SMILES: CCCCCCCC(CCCCC)CCCCC(=O)OCCCCCCN(CCCCCO)CC(O)CCCCOC(=O)CCCCC(CCCCC)CCCCCCC] |
| 1-v | 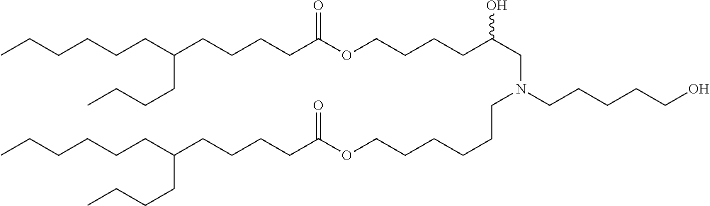 [SMILES: CCCCCCC(CCCC)CCCCC(=O)OCCCCCCN(CCCCCO)CC(O)CCCCOC(=O)CCCCC(CCCC)CCCCCC] |
| 1-w | 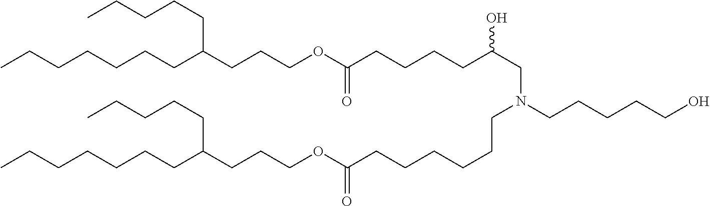 [SMILES: CCCCCCCC(CCCCC)CCCOC(=O)CCCCCCN(CCCCCO)CC(O)CCCCC(=O)OCCCC(CCCCC)CCCCCCC] |
| 1-x | 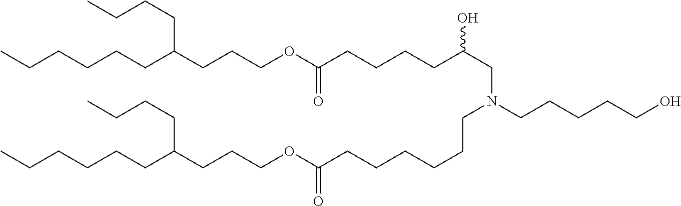 [SMILES: CCCCCCC(CCCC)CCCOC(=O)CCCCCCN(CCCCCO)CC(O)CCCCC(=O)OCCCC(CCCC)CCCCCC] |
| 1-y | 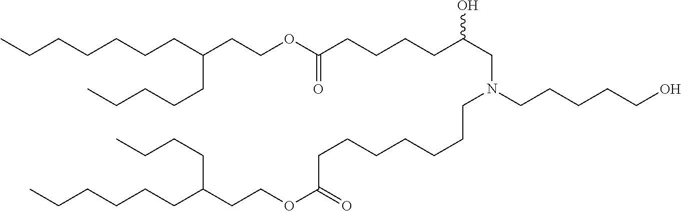 [SMILES: CCCCCCCC(CCCCC)CCOC(=O)CCCCC(O)CN(CCCCCO)CCCCCCCC(=O)OCCC(CCCC)CCCCCC] |
| 1-z | 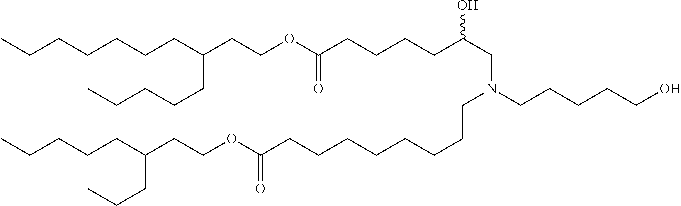 [SMILES: CCCCCCCC(CCCCC)CCOC(=O)CCCCC(O)CN(CCCCCO)CCCCCCCCC(=O)OCCC(CCC)CCCCC] |
| 1-aa | 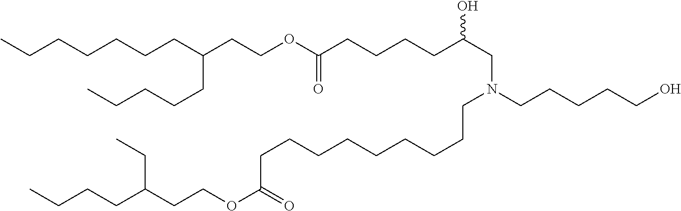 [SMILES: CCCCCCCC(CCCCC)CCOC(=O)CCCCC(O)CN(CCCCCO)CCCCCCCCCC(=O)OCCC(CC)CCCC] |
| 1-ab | 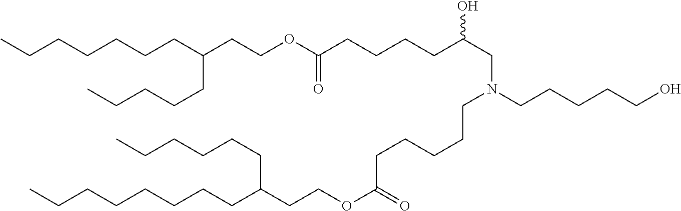 [SMILES: CCCCCCCCC(CCCCCC)CCOC(=O)CCCCCN(CCCCCO)CC(O)CCCCC(=O)OCCC(CCCCC)CCCCCCC] |
| 1-ac | 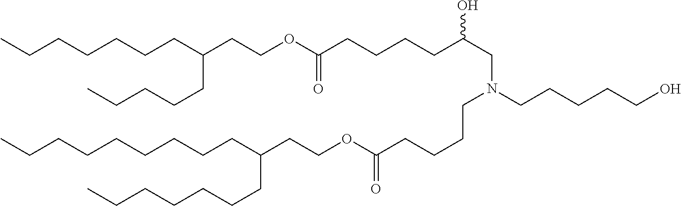 [SMILES: CCCCCCCCCC(CCCCCCC)CCOC(=O)CCCCN(CCCCCO)CC(O)CCCCC(=O)OCCC(CCCCC)CCCCCCC] |
| 1-ad | 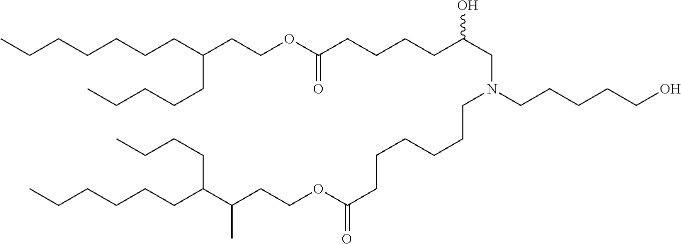 [SMILES: CCCCCCCC(CCCCC)CCOC(=O)CCCCC(O)CN(CCCCCO)CCCCCCC(=O)OCCC(C)C(CCCC)CCCCCC] |
| 1-ae | 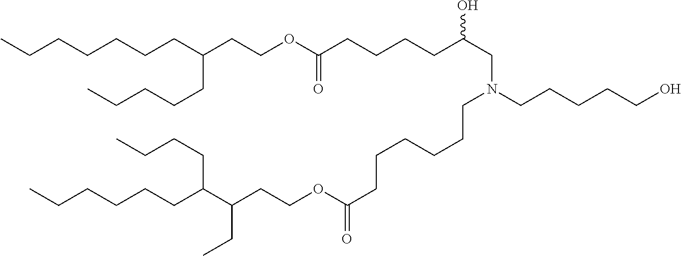 [SMILES: CCCCCCCC(CCCCC)CCOC(=O)CCCCC(O)CN(CCCCCO)CCCCCCC(=O)OCCC(CC)C(CCCC)CCCCCC] |
| 1-af | 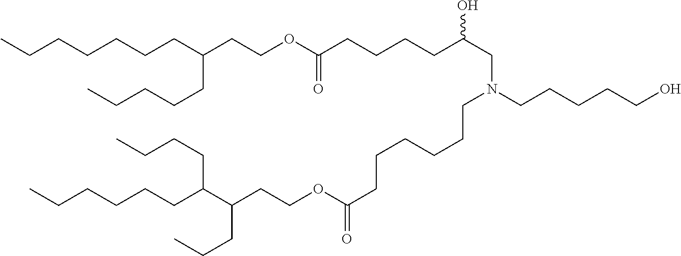 [SMILES: CCCCCCCC(CCCCC)CCOC(=O)CCCCC(O)CN(CCCCCO)CCCCCCC(=O)OCCC(CCC)C(CCCC)CCCCCC] |
| 1-ag | 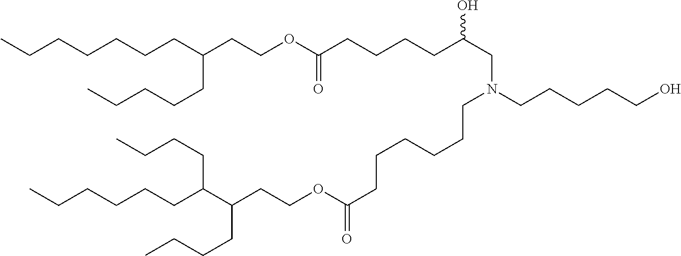 [SMILES: CCCCCCCC(CCCCC)CCOC(=O)CCCCC(O)CN(CCCCCO)CCCCCCC(=O)OCCC(CCCC)C(CCCC)CCCCCC] |
| 1-ah | 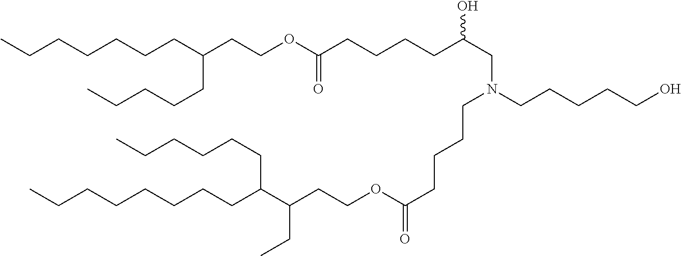 [SMILES: CCCCCCCCC(CCCCCC)C(CC)CCOC(=O)CCCCN(CCCCCO)CC(O)CCCCC(=O)OCCC(CCCCC)CCCCCCC] |
| 1-ai | 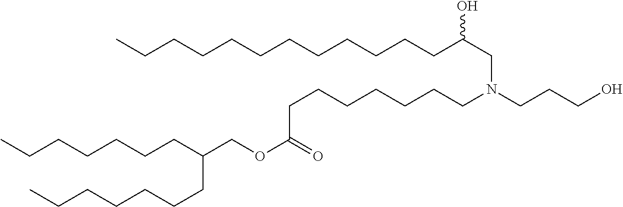 [SMILES: CCCCCCCCCCCCC(O)CN(CCCO)CCCCCCCC(=O)OCC(CCCCCCC)CCCCCCC] |
| 1-aj | 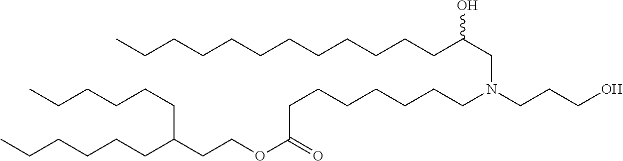 [SMILES: CCCCCCCCCCCCC(O)CN(CCCO)CCCCCCCC(=O)OCCC(CCCCCC)CCCCCC] |
| 1-ak | 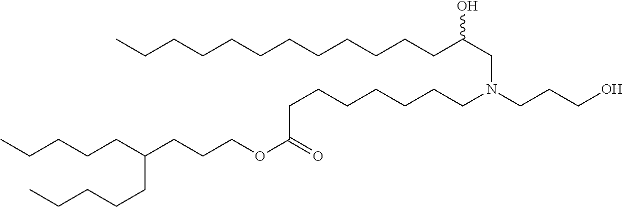 [SMILES: CCCCCCCCCCCCC(O)CN(CCCO)CCCCCCCC(=O)OCCCC(CCCCC)CCCCC] |

In some embodiments, an ionizable lipid of the present disclosure is represented by Formula (22):

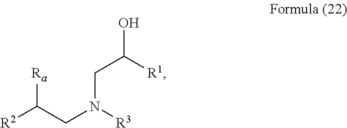

or is a pharmaceutically acceptable salt thereof, wherein:
   R^{a} is hydrogen or hydroxyl;
   R¹ is C₁-C₃₀ alkyl or R¹*
   R² is C₁-C₃₀ alkyl or R²*;
   R¹* and R²* are independently selected from:
      (CH₂)_{q}C(O)O(CH₂)ᵣC(R⁴)(R⁵)(R⁶),
      (CH₂)_{q}OC(O)(CH₂)ᵣC(R⁴)(R⁵)(R⁶), and
      (CH₂)_{q}OC(O)O(CH₂)ᵣC(R⁴)(R⁵)(R⁶);
   wherein:
      q is an integer from 0 to 12,
      r is an integer from 0 to 6, wherein at least one occurrence of r is not 0;
      R⁴ is hydrogen or R⁷;
      R⁵, R⁶, and R⁷ are each independently C₁-C₂₀ alkyl or C₂-C₂₀-alkenyl;
   wherein (i) R¹ is R¹*, (ii) R² is R²*, or (iii) R¹ is R¹* and R² is R²*; and
   R³ is L-R′, wherein L is linear or branched C₁-C₁₀ alkylene, and R′ is (i) mono- or bicyclic heterocyclyl or heteroaryl, such as imidazolyl, pyrazolyl, 1,2,4-triazolyl, or benzimidazolyl, each optionally substituted at one or more available carbon and nitrogen by C₁-C₆ alkyl, or (ii) R^{A}, R^{B}, or R^{C}, wherein
      R^{A} is selected from:

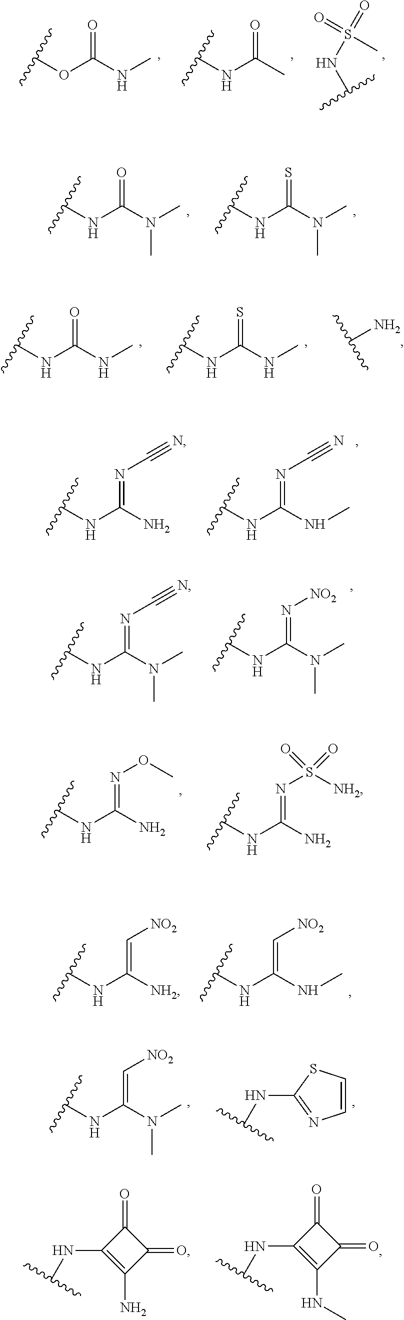

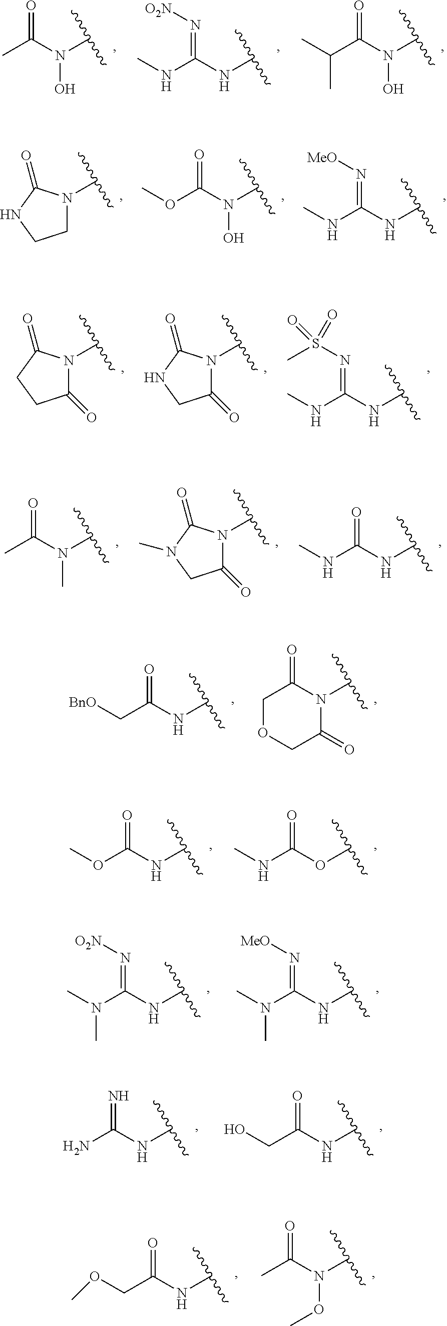

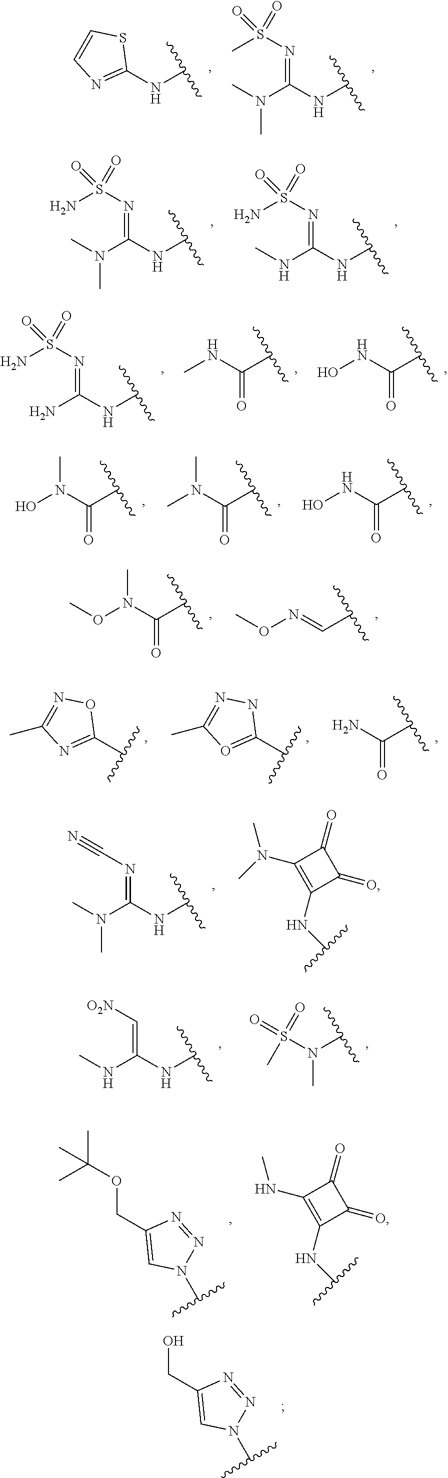

RB is selected from:

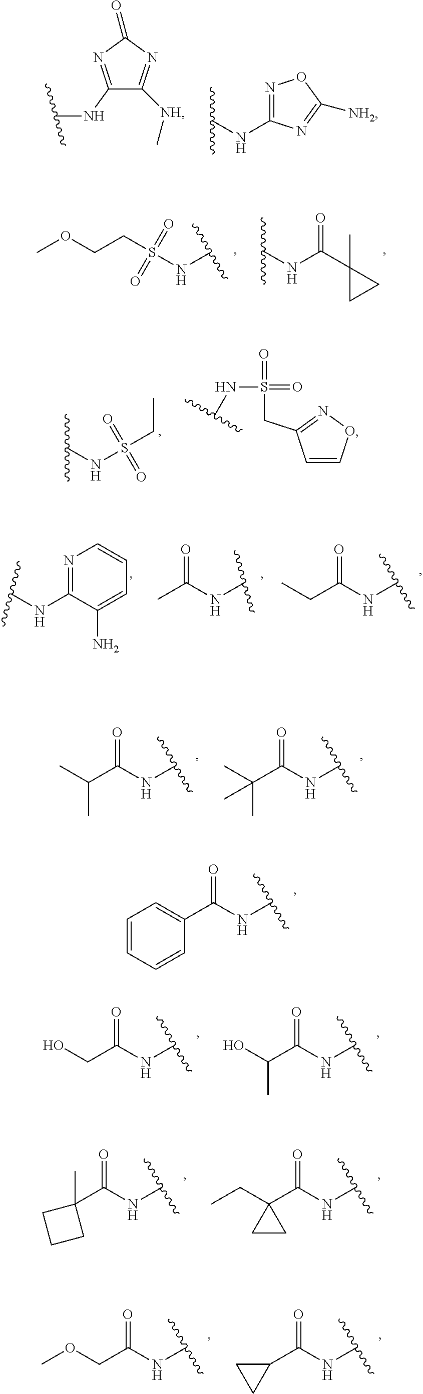

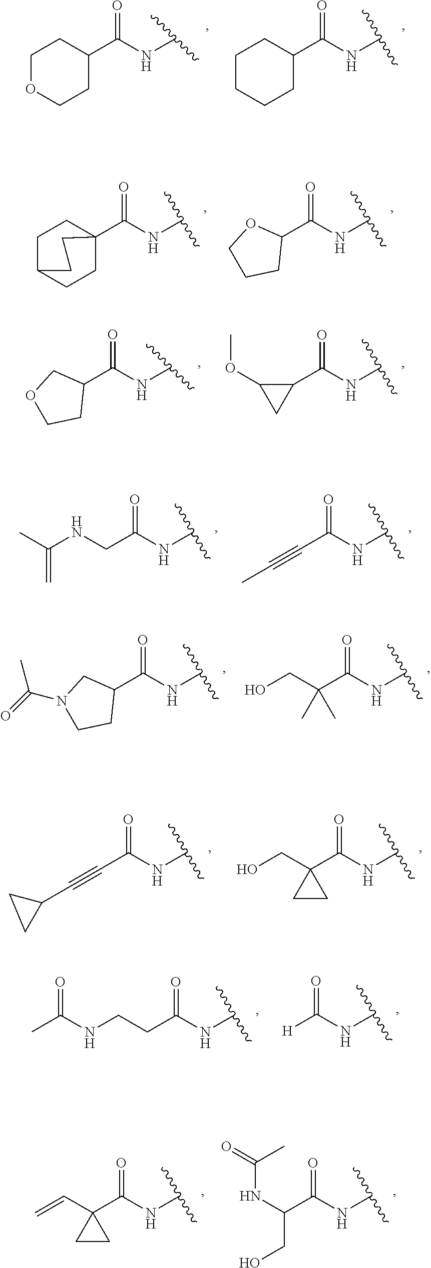

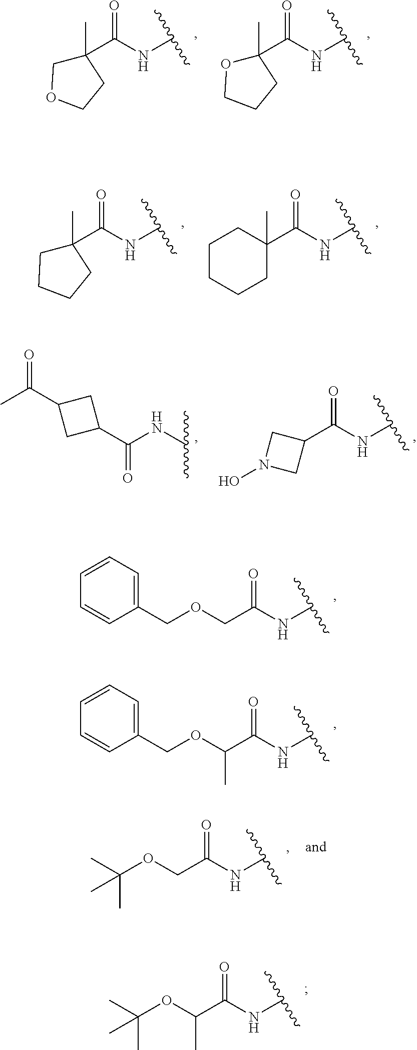

and
R^{C} is selected from:

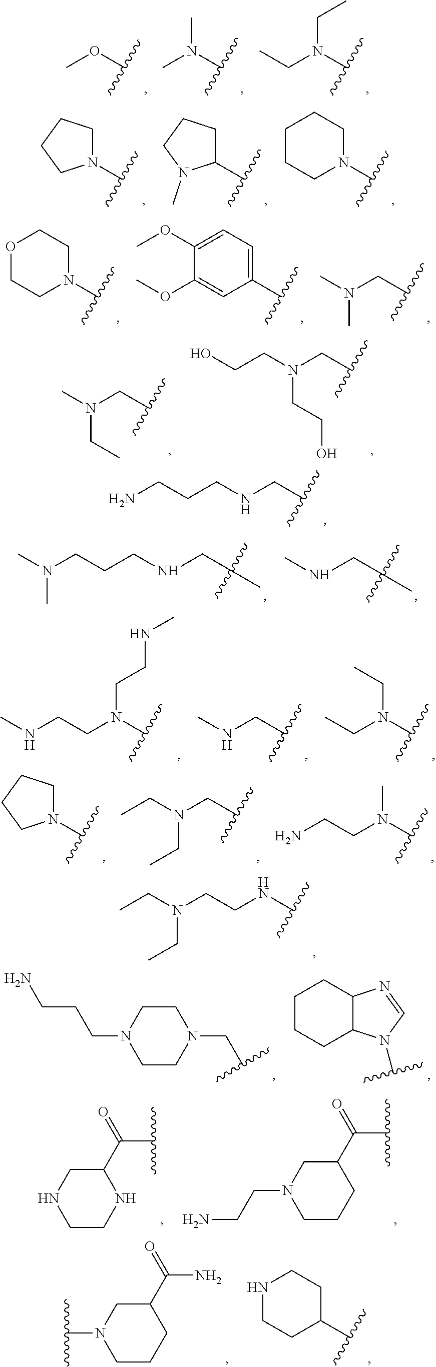

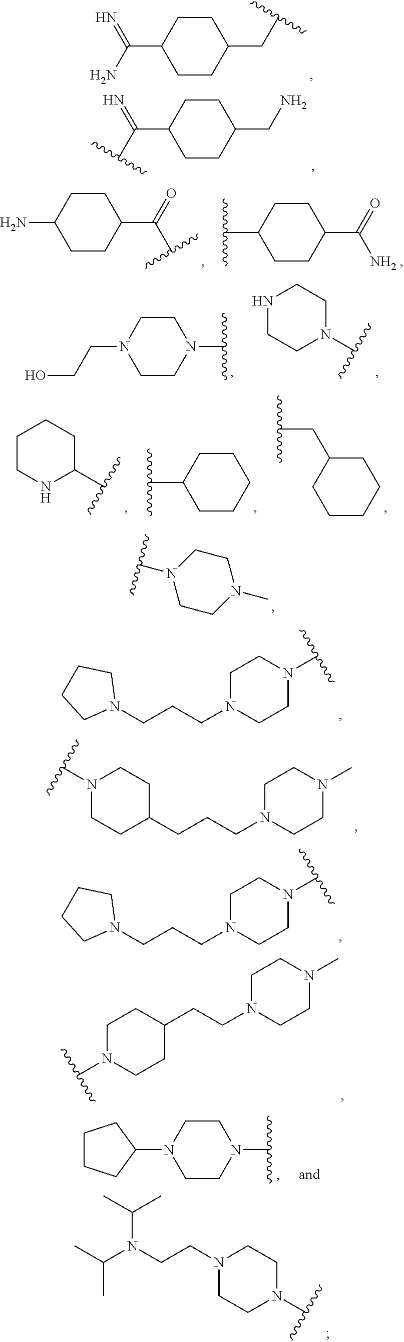

with the proviso that the ionizable lipid is not:

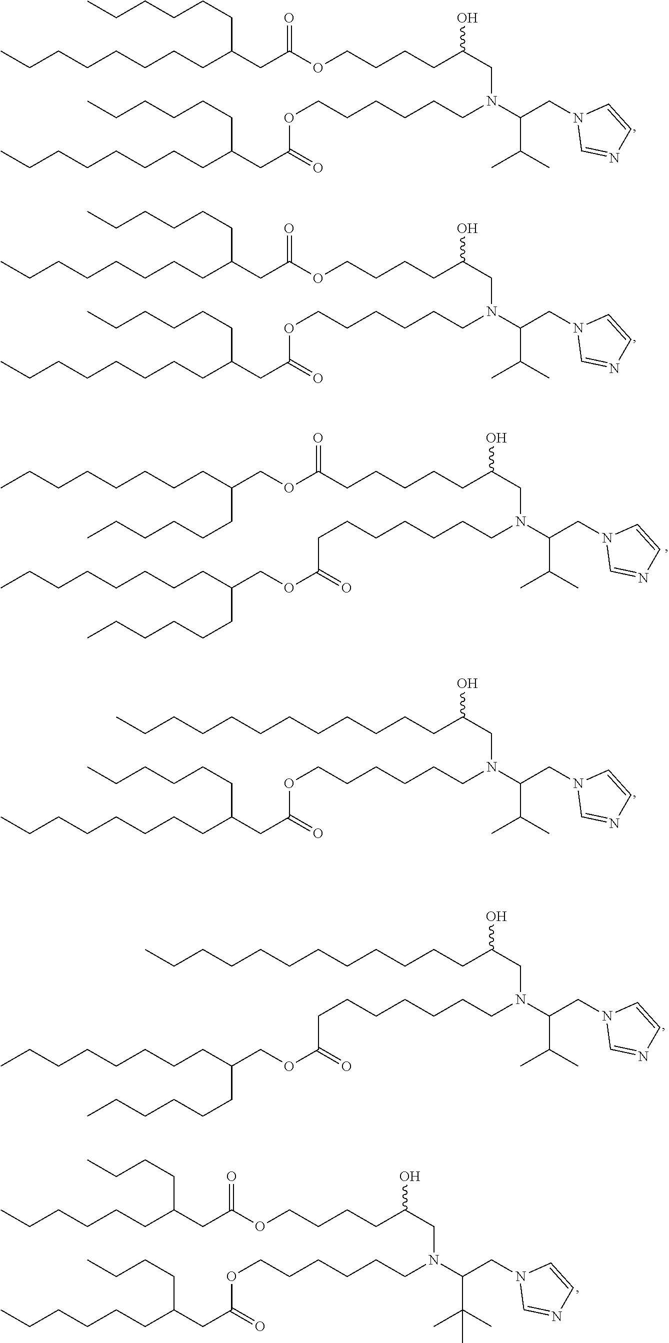

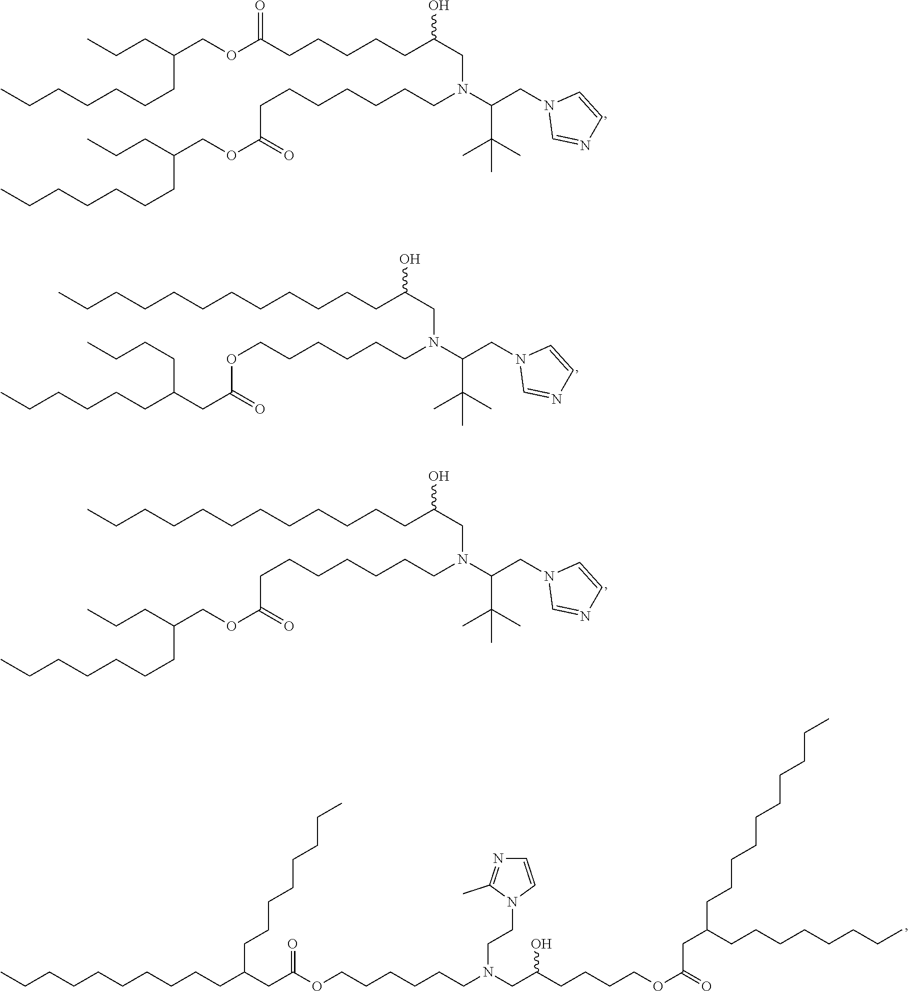 [SMILES: CCCCCCCC(CCC)COC(=O)CCCCCCCN(CC(O)CCCCCC(=O)OCC(CCC)CCCCCCC)C(Cn1ccnc1)C(C)(C)C ; CCCCCCCCCCCCC(O)CN(CCCCCCOC(=O)CC(CCCC)CCCCCC)C(Cn1ccnc1)C(C)(C)C ; CCCCCCCCCCCCC(O)CN(CCCCCCCC(=O)OCC(CCC)CCCCCCC)C(Cn1ccnc1)C(C)(C)C ; CCCCCCCCCCC(CCCCCCCC)CC(=O)OCCCCCCN(CCn1ccnc1C)CC(O)CCCCOC(=O)CC(CCCCCCCC)CCCCCCCCCC]

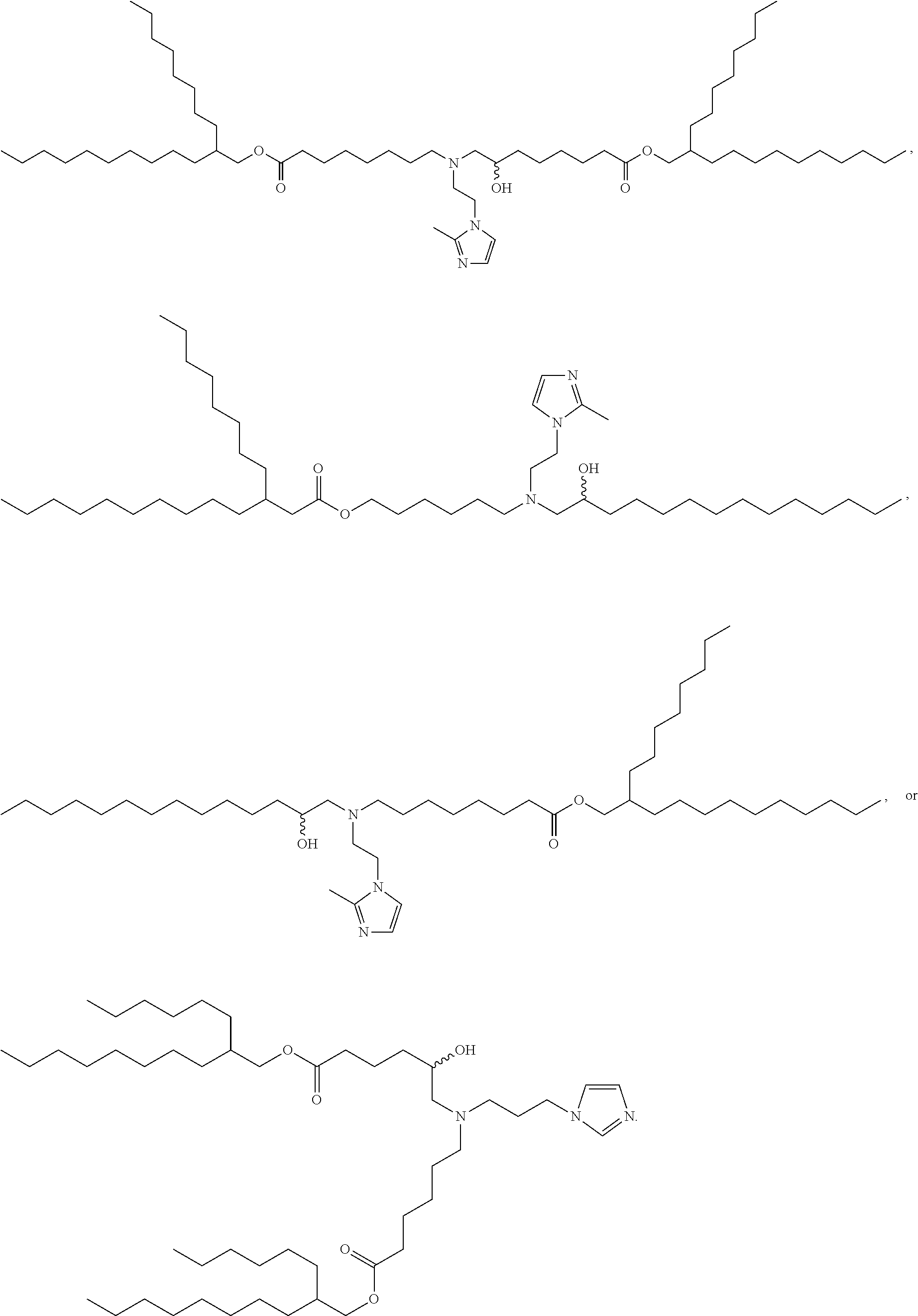

In some embodiments of Formula (22), R³ is selected from:

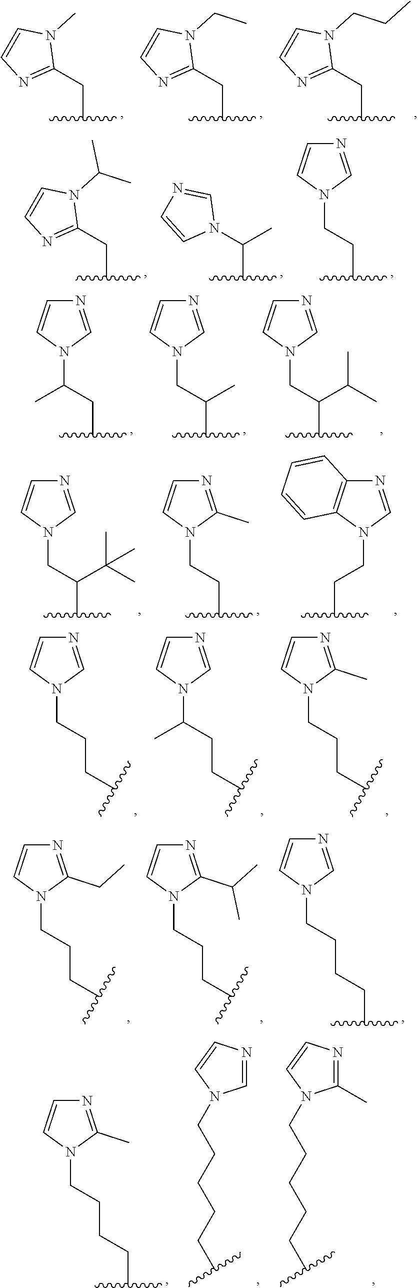

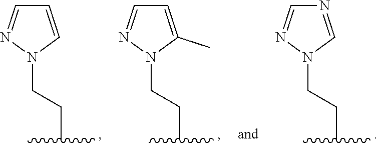

In some embodiments of Formula (22), R¹ is R¹*, R² is R²*, and the ionizable lipid is of Formula (23):

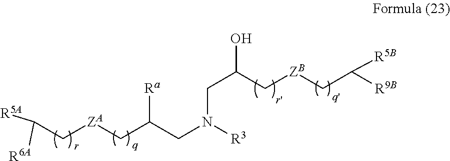

wherein:
q and q′ are each independently integers from 0 to 12;
r and r′ are each independently integers from 0 to 6, wherein at least one of r or r′ is not 0; Z^{A} and Z^{B} are each independently selected from ∧—C(O)O—, ∧—OC(O), and —OC(O)O—; where ∧ denotes the attachment point to —(CH₂)_{q}— or —(CH₂)_{q}′-; and
R^{5A}, R^{5B}, R^{6A} and R^{6B} are each independently C₁-C₂₀ alkyl or C₂-C₂₀ alkenyl.

In some embodiments of Formula (23), Z^{A} and Z^{B} are ∧—C(O)O—, and the ionizable lipid is of Formula (23a-1):

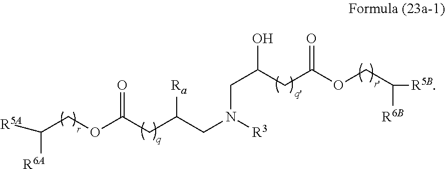

In some embodiments of Formula (23), Z^{A} and Z^{B} are ∧—OC(O)—, and the ionizable lipid is of Formula (23a-2)

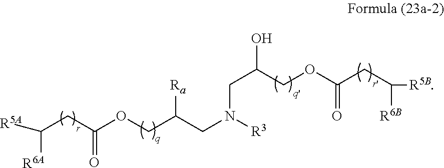

In some embodiments of Formula (23), Z^{A} and Z^{B} are —O(C)(O)O—, and the ionizable lipid is represented by Formula (23a-3):

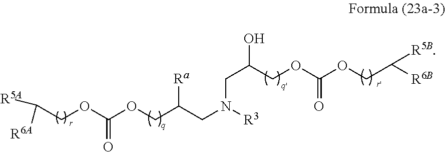

In some embodiments of Formula (22), R² is C₁-C₃₀ alkyl, and the ionizable lipid is of Formula (25):

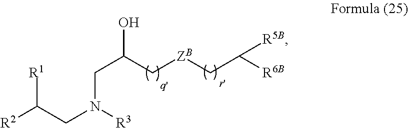

or is a pharmaceutically acceptable salt thereof, wherein:
   Z^{B} is selected from ∧—C(O)O—, ∧—OC(O)—, and —OC(O)O—; where ∧ denotes the attachment point to —(CH₂)_{q}′-;
   R^{5B} and R^{6B} are each independently C₁-C₂₀ alkyl or C₂-C₂₀ alkenyl;
   q′ is an integer from 0 to 12; and
   r′ is an integer from 1 to 6.

In some embodiments of Formula (25), Z^{B} is ∧—C(O)O—, and the ionizable lipid is of Formula (25a-1):

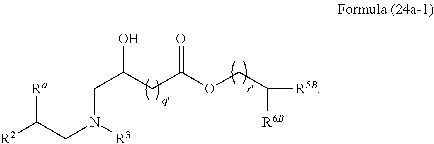

In some embodiments of Formula (25), Z^{B} is ∧—OC(O)—, and the ionizable lipid is of Formula (25a-2):

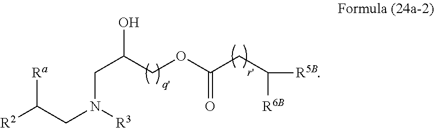

In some embodiments of Formula (25), Z^{B} is —OC(O)O—, and the ionizable lipid is of Formula (25a-3):

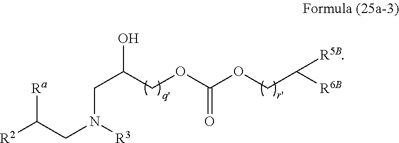

In some embodiments, an ionizable lipid is selected from the table below:

| Lipid | Structure |
|---|---|
| 100-a | 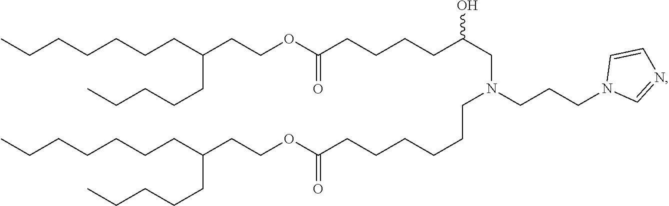 [SMILES: CCCCCCCC(CCCCC)CCOC(=O)CCCCCCN(CCCn1ccnc1)CC(O)CCCCC(=O)OCCC(CCCCC)CCCCCCC] |
| 100-b | 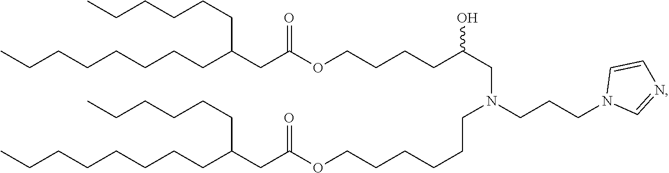 [SMILES: CCCCCCCCC(CCCCCC)CC(=O)OCCCCCCN(CCCn1ccnc1)CC(O)CCCCOC(=O)CC(CCCCCC)CCCCCCCC] |
| 100-c | 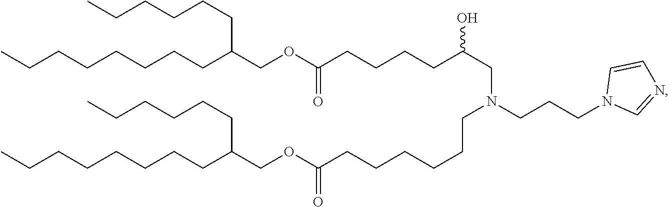 [SMILES: CCCCCCCCC(CCCCCC)COC(=O)CCCCCCN(CCCn1ccnc1)CC(O)CCCCC(=O)OCC(CCCCCC)CCCCCCCC] |
| 100-d | 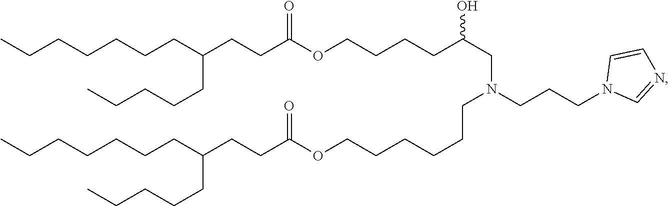 [SMILES: CCCCCCCC(CCCCC)CCC(=O)OCCCCCCN(CCCn1ccnc1)CC(O)CCCCOC(=O)CCC(CCCCC)CCCCCCC] |
| 100-e | 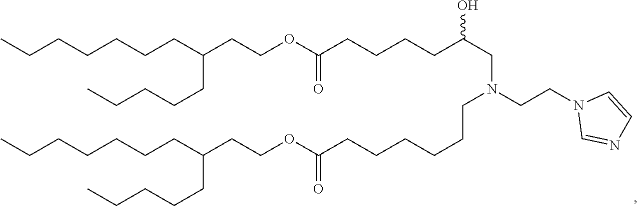 [SMILES: CCCCCCCC(CCCCC)CCOC(=O)CCCCCCN(CCn1ccnc1)CC(O)CCCCC(=O)OCCC(CCCCC)CCCCCCC] |
| | and |
| 100-f | 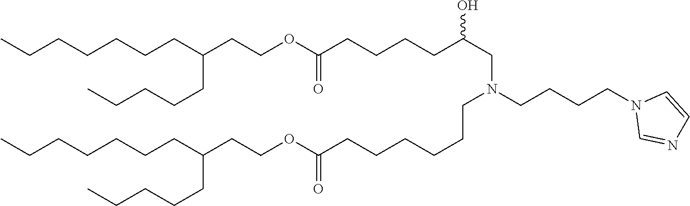 [SMILES: CCCCCCCC(CCCCC)CCOC(=O)CCCCCCN(CCCCn1ccnc1)CC(O)CCCCC(=O)OCCC(CCCCC)CCCCCCC] |

In some embodiments, an ionizable lipid is selected from the table below.

| Lipid | Structure |
|---|---|
| 1-al | 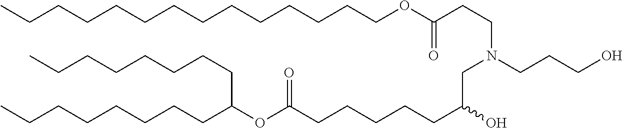 [SMILES: CCCCCCCCCCCCCCOC(=O)CCN(CCCO)CC(O)CCCCCC(=O)OC(CCCCCCCC)CCCCCCCC] |
| 1-am | 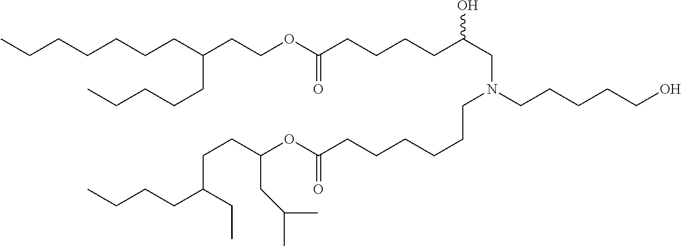 [SMILES: CCCCCCCC(CCCCC)CCOC(=O)CCCCC(O)CN(CCCCCO)CCCCCCC(=O)OC(CCC(CC)CCCC)CC(C)C] |
| 1-an | 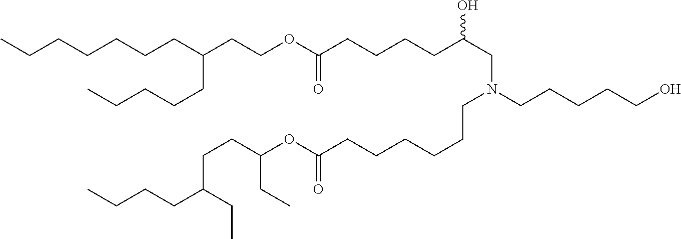 [SMILES: CCCCCCCC(CCCCC)CCOC(=O)CCCCC(O)CN(CCCCCO)CCCCCCC(=O)OC(CC)CCC(CC)CCCC] |
| 1-ao | 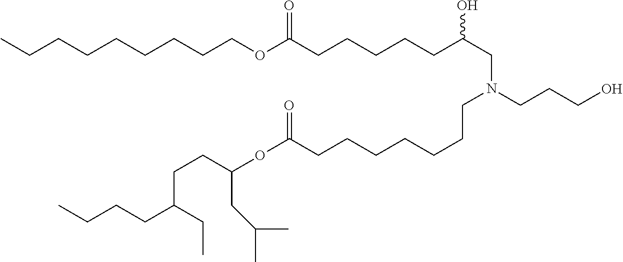 [SMILES: CCCCCCCCCOC(=O)CCCCCC(O)CN(CCCO)CCCCCCCC(=O)OC(CCC(CC)CCCC)CC(C)C] |
| 1-ap | 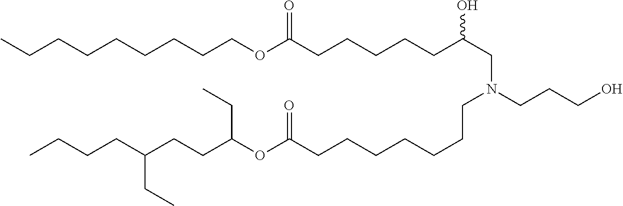 [SMILES: CCCCCCCCCOC(=O)CCCCCC(O)CN(CCCO)CCCCCCCC(=O)OC(CC)CCC(CC)CCCC] |
| 1-aq | 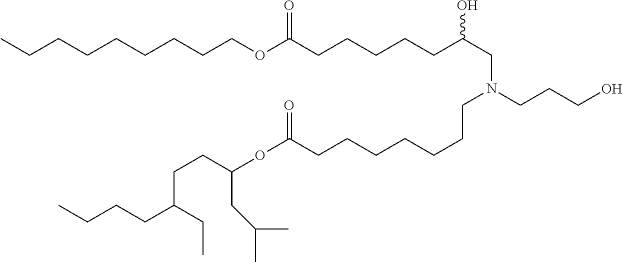 [SMILES: CCCCCCCCCOC(=O)CCCCCC(O)CN(CCCO)CCCCCCCC(=O)OC(CCC(CC)CCCC)CC(C)C] |
| 1-ar | 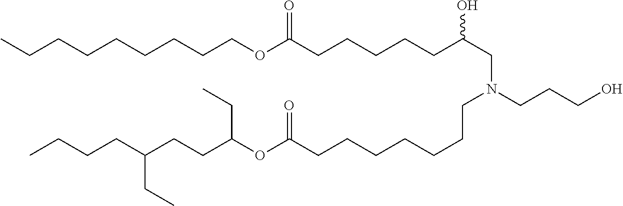 [SMILES: CCCCCCCCCOC(=O)CCCCCC(O)CN(CCCO)CCCCCCCC(=O)OC(CC)CCC(CC)CCCC] |
| 1-as | 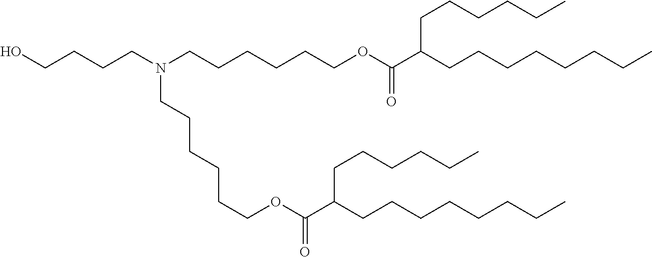 [SMILES: CCCCCCCCC(CCCCCC)C(=O)OCCCCCCN(CCCCO)CCCCCCOC(=O)C(CCCCCC)CCCCCCCC] |
| 1-at | 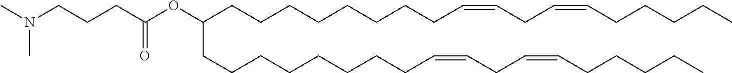 [SMILES: CCCCC/C=C\C/C=C\CCCCCCCCC(CCCCCCCC/C=C\C/C=C\CCCCC)OC(=O)CCCN(C)C] |
| 1-au | 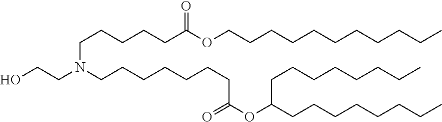 [SMILES: CCCCCCCCCCCOC(=O)CCCCCN(CCO)CCCCCCCC(=O)OC(CCCCCCCC)CCCCCCCC] |

In some embodiments, an ionizable lipid is described in US patent publication number 20190321489. In some embodiments, an ionizable lipid is described in international patent publication WO 2010/053572, incorporated herein by reference. In some embodiments, an ionizable lipid is C12-200, described at paragraph of WO 2010/053572.

Several ionizable lipids have been described in the literature, many of which are commercially available. In certain embodiments, such ionizable lipids are included in the transfer vehicles described herein. In some embodiments, the ionizable lipid N-[1-(2,3-dioleyloxy) propyl]-N,N,N-trimethylammonium chloride or “DOTMA” is used. (Felgner et al. Proc. Nat'l Acad. Sci. 84, 7413 (1987); U.S. Pat. No. 4,897,355). DOTMA can be formulated alone or can be combined with a neutral lipid, dioleoylphosphatidylethanolamine or “DOPE” or other cationic or non-cationic lipids into a lipid nanoparticle. Other suitable cationic lipids include, for example, ionizable cationic lipids as described in U.S. provisional patent application 61/617,468, filed Mar. 29, 2012 (incorporated herein by reference), such as, e.g., (15Z,18Z)—N,N-dimethyl-6-(9Z, 12Z)-octadeca-9,12-dien-1-yl)tetracosa-15,18-dien-1-amine (HGT5000), (15Z,18Z)—N,N-dimethyl-6-((9Z,12Z)-octadeca-9,12-dien-1-yl)tetracosa-4,15,18-trien-1-amine (HGT5001), and (15Z,18Z)—N,N-dimethyl-6-((9Z,12Z)-octadeca-9,12-dien-1-yl)tetracosa-5,15,18-trien-1-amine (HGT5002), C12-200 (described in WO 2010/053572), 2-(2,2-di((9Z,12Z)-octadeca-9,12-dien-1-yl)-1,3-dioxolan-4-yl)-N,N-dimethylethanamine (DLinKC2-DMA)) (See, WO 2010/042877; Semple et al., Nature Biotech. 28:172-176 (2010)), 2-(2,2-di((9Z,2Z)-octadeca-9,12-dien-1-yl)-1,3-dioxolan-4-yl)-N,N-dimethylethanamine (DLin-KC2-DMA), (3S,10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl) propanoate (ICE), (15Z,18Z)—N,N-dimethyl-6-(9Z,12Z)-octadeca-9,12-dien-1-yl)tetracosa-15,18-dien-1-amine (HGT5000), (15Z,18Z)—N,N-dimethyl-6-((9Z,12Z)-octadeca-9,12-dien-1-yl)tetracosa-4,15,18-trien-1-amine (HGT5001), (15Z,18 Z)—N,N-dimethyl-6-((9Z,12Z)-octadeca-9,12-dien-1-yl)tetracosa-5,15,18-trien-1-amine (HGT5002), 5-carboxyspermylglycine-dioctadecylamide (DOGS), 2,3-dioleyloxy-N-[2 (spermine-carboxamido)ethyl]-N,N-dimethyl-1-propanaminium (DOSPA) (Behr et al. Proc. Nat.′1 Acad. Sci. 86, 6982 (1989); U.S. Pat. Nos. 5,171,678; 5,334,761), 1,2-Dioleoyl-3-Dimethylammonium-Propane (DODAP), 1,2-Dioleoyl-3-Trimethylammonium-Propane or (DOTAP). Contemplated ionizable lipids also include 1,2-distcaryloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), N-dioleyl-N,N-dimethylammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-octadecadienoxy) propane (CLinDMA), 2-[5′-(cholest-5-en-3-beta-oxy)-3′-oxapentoxy)-3-dimethyl-1-(cis,cis-9′, 1-2′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylamninopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminocthyl-[1,3]-dioxolane (DLin-K-XTC2-DMA) or GL67, or mixtures thereof. (Heyes, J., et al., J Controlled Release 107:276-287 (2005); Morrissey, D V., et al., Nat. Biotechnol. 23 (8): 1003-1007 (2005); PCT Publication WO2005/121348A1). The use of cholesterol-based ionizable lipids to formulate the transfer vehicles (e.g., lipid nanoparticles) is also contemplated herein. Such cholesterol-based ionizable lipids can be used, either alone or in combination with other lipids. Suitable cholesterol-based ionizable lipids include, for example, DC-Cholesterol (N,N-dimethyl-N-ethylcarboxamidocholesterol), and 1,4-bis(3-N-oleylamino-propyl) piperazine (Gao, et al., Biochem. Biophys. Res. Comm. 179, 280 (1991); Wolf et al. BioTechniques 23, 139 (1997); U.S. Pat. No. 5,744,335).

Also contemplated are cationic lipids such as dialkylamino-based, imidazole-based, and guanidinium-based lipids. For example, also contemplated is the use of the ionizable lipid (3S,10R, 13R, 17R)-10,13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl) propanoate (ICE), as disclosed in International Application No. PCT/US2010/058457, incorporated herein by reference.

Also contemplated are ionizable lipids such as the dialkylamino-based, imidazole-based, and guanidinium-based lipids.

In some embodiments, an ionizable lipid is described by US patent publication number 20190314284.

The ionizable lipids include those disclosed in international patent application PCT/US2019/025246, and US patent publications 2017/0190661 and 2017/0114010, incorporated herein by reference in their entirety.

In some embodiments, an ionizable lipid is as described in international patent application PCT/US2019/015913.

Preparation methods for the above compounds and compositions are described herein below and/or known in the art.

It will be appreciated by those skilled in the art that in the process described herein the functional groups of intermediate compounds may need to be protected by suitable protecting groups. Such functional groups include, e.g., hydroxyl, amino, mercapto, and carboxylic acid. Suitable protecting groups for hydroxyl include, e.g., trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino, and guanidino include, e.g., t-butoxycarbonyl, benzyloxycarbonyl, and the like. Suitable protecting groups for mercapto include, e.g., —C(O)—R″ (where R″ is alkyl, aryl, or arylalkyl), p-methoxybenzyl, trityl, and the like. Suitable protecting groups for carboxylic acid include, e.g., alkyl, aryl, or arylalkyl esters. Protecting groups may be added or removed in accordance with standard techniques, which are known to one skilled in the art and as described herein. The use of protecting groups is described in detail in, e.g., Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin, or a 2-chlorotrityl-chloride resin.

It will also be appreciated by those skilled in the art, although such protected derivatives of compounds described herein may not possess pharmacological activity as such, they may be administered to a mammal and thereafter metabolized in the body to form compounds described herein which are pharmacologically active. Such derivatives may therefore be described as prodrugs. All prodrugs of compounds described herein are included within the scope of the present disclosure.

Furthermore, all compounds described herein which exist in free base or acid form can be converted to their pharmaceutically acceptable salts by treatment with the appropriate inorganic or organic base or acid by methods known to one skilled in the art. Salts of the compounds described herein can also be converted to their free base or acid form by standard techniques.

### Amine Lipids

In certain embodiments, transfer vehicle compositions for the delivery of circular RNA comprise an amine lipid. In certain embodiments, an ionizable lipid is an amine lipid. In some embodiments, an amine lipid is described in international patent application PCT/US2018/053569.

Amine lipids and other biodegradable lipids suitable for use in the transfer vehicles, e.g., lipid nanoparticles, described herein are biodegradable in vivo. The amine lipids described herein have low toxicity (e.g., are tolerated in animal models without adverse effect in amounts of greater than or equal to 10 mg/kg). In certain embodiments, transfer vehicles composing an amine lipid include those where at least 75% of the amine lipid is cleared from the plasma within 8, 10, 12, 24, or 48 hours, or 3, 4, 5, 6, 7, or 10 days.

Biodegradable lipids include, for example, the biodegradable lipids of WO2017/173054, WO2015/095340, and WO2014/136086.

Lipid clearance may be measured by methods known by persons of skill in the art. See, for example, Maier, M. A., et al. Biodegradable Lipids Enabling Rapidly Eliminated Lipid Nanoparticles for Systemic Delivery of RNAi Therapeutics. Mol. Ther. 2013, 21 (8), 1570-78.

Transfer vehicle compositions comprising an amine lipid can lead to an increased clearance rate. In some embodiments, the clearance rate is a lipid clearance rate, for example the rate at which a lipid is cleared from the blood, serum, or plasma. In some embodiments, the clearance rate is an RNA clearance rate, for example the rate at which a circular RNA is cleared from the blood, serum, or plasma. In some embodiments, the clearance rate is the rate at which transfer vehicles are cleared from the blood, serum, or plasma. In some embodiments, the clearance rate is the rate at which transfer vehicles are cleared from a tissue, such as liver tissue or spleen tissue. In certain embodiments, a high rate of clearance leads to a safety profile with no substantial adverse effects. The amine lipids and biodegradable lipids may reduce transfer vehicle accumulation in circulation and in tissues. In some embodiments, a reduction in transfer vehicle accumulation in circulation and in tissues leads to a safety profile with no substantial adverse effects.

Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood, where pH is approximately 7.35, the lipid, such as an amine lipid, may not be protonated and thus bear no charge. The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g., to the liver. Further, it has been found that lipids with a pKa ranging from about 5.3 to about 6.4 are effective for delivery in vivo, e.g., into tumors. See, e.g., WO2014/136086.

### Lipids Containing a Disulfide Bond

In some embodiments, the ionizable lipid is described in U.S. Pat. No. 9,708,628.

In some embodiments, the lipid may have an —S—S— (disulfide) bond. The production method for such a compound includes, for example, a method including producing
R^{3a}—(Y^{a}—R^{2a})n^{a}—X^{a}—R^{1a}—SH, and
R^{3b}—(Y^{b}—R^{2b})n^{b}—X^{b}—R^{1b}—SH, and
   subjecting them to oxidation (coupling) to give a compound containing —S—S—, a method including sequentially bonding necessary parts to a compound containing an —S—S— bond to finally obtain the compound and the like. Preferred is the latter method.

### Further Exemplary Ionizable Lipids

In some embodiments, an ionizable lipid is described in U.S. Pat. No. 9,765,022.

In some embodiments, other lipid-like compounds can be prepared using suitable routes known in the art. The methods can include an additional step(s) to add or remove suitable protecting groups in order to ultimately allow synthesis of the lipid-like compounds. In addition, various synthetic steps can be performed in an alternate sequence or order to give the desired material. Synthetic chemistry transformations and protecting group methodologies (protection and deprotection) useful in synthesizing applicable lipid-like compounds are known in the art, including, for example, R. Larock, Comprehensive Organic Transformations (2nd Ed., VCH Publishers 1999); P. G. M. Wuts and T. W. Greene, Greene's Protective Groups in Organic Synthesis (4th Ed., John Wiley and Sons 2007); L. Fieser and M. Fieser, Fieser and Fieser's Reagents for Organic Synthesis (John Wiley and Sons 1994); and L. Paquette, ed., Encyclopedia of Reagents for Organic Synthesis (2nd ed., John Wiley and Sons 2009) and subsequent editions thereof. Certain lipid-like compounds may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

As mentioned above, these lipid-like compounds are useful for delivery of pharmaceutical agents. They can be preliminarily screened for their efficacy in delivering pharmaceutical agents by an in vitro assay and then confirmed by animal experiments and clinic trials. Other methods will also be apparent to those of ordinary skill in the art.

The above described complexes can be prepared using procedures described in publications such as Wang et al., ACS Synthetic Biology, 1, 403-07 (2012). Generally, they are obtained by incubating a lipid-like compound and a pharmaceutical agent in a buffer such as a sodium acetate buffer or a phosphate buffered saline (“PBS”).

### Hydrophilic Groups

In certain embodiments, the selected hydrophilic functional group or moiety may alter or otherwise impart properties to the compound or to the transfer vehicle of which such compound is a component (e.g., by improving the transfection efficiencies of a lipid nanoparticle of which the compound is a component). For example, the incorporation of guanidinium as a hydrophilic head-group in the compounds disclosed herein may promote the fusogenicity of such compounds (or of the transfer vehicle of which such compounds are a component) with the cell membrane of one or more target cells, thereby enhancing, for example, the transfection efficiencies of such compounds. It has been hypothesized that the nitrogen from the hydrophilic guanidinium moiety forms a six-membered ring transition state which grants stability to the interaction and thus allows for cellular uptake of encapsulated materials. (Wender, et al., Adv. Drug Del. Rev. (2008) 60:452-472.) Similarly, the incorporation of one or more amino groups or moieties into the disclosed compounds (e.g., as a head-group) may further promote disruption of the endosomal/lysosomal membrane of the target cell by exploiting the fusogenicity of such amino groups. This is based not only on the pKa of the amino group of the composition, but also on the ability of the amino group to undergo a hexagonal phase transition and fuse with the target cell surface, i.e., the vesicle membrane. (Koltover, et al. Science (1998) 281:78-81.) The result is believed to promote the disruption of the vesicle membrane and release of the lipid nanoparticle contents into the target cell.

Similarly, in certain embodiments the incorporation of, for example, imidazole as a hydrophilic head-group in the compounds disclosed herein may serve to promote endosomal or lysosomal release of, for example, contents that are encapsulated in a transfer vehicle (e.g., lipid nanoparticle). Such enhanced release may be achieved by one or both of a proton-sponge mediated disruption mechanism and/or an enhanced fusogenicity mechanism. The proton-sponge mechanism is based on the ability of a compound, and in particular a functional moiety or group of the compound, to buffer the acidification of the endosome. This may be manipulated or otherwise controlled by the pKa of the compound or of one or more of the functional groups comprising such compound (e.g., imidazole). Accordingly, in certain embodiments the fusogenicity of, for example, the imidazole-based compounds disclosed herein (e.g., HGT4001 and HGT4004) are related to the endosomal disruption properties, which are facilitated by such imidazole groups, which have a lower pKa relative to other traditional ionizable lipids. Such endosomal disruption properties in turn promote osmotic swelling and the disruption of the liposomal membrane, followed by the transfection or intracellular release of the polynucleotide materials loaded or encapsulated therein into the target cell. This phenomenon can be applicable to a variety of compounds with desirable pKa profiles in addition to an imidazole moiety. Such embodiments also include multi-nitrogen based functionalities such as polyamines, poly-peptide (histidine), and nitrogen-based dendritic structures.

Exemplary ionizable and/or cationic lipids are described in International PCT patent publications WO2015/095340, WO2015/199952, WO2018/011633, WO2017/049245, WO2016/081029, WO2015/061467, WO2012/040184, WO2012/000104, WO2015/074085, WO2017/004 143, WO2017/075531, WO2017/117528, WO2011/022460, WO2013/148541, WO2013/116126, WO2011/153120, WO2012/044638, WO2012/054365 WO2011/090965, WO2013/016058, WO2012/162210, WO2008/042973, WO2010/129709, WO2010/144740, WO20 12/099755, WO2013/049328, WO2013/086322, WO2013/086373, WO2011/071860, WO2009/132131, WO2010/048536, WO2010/088537, WO2010/054401, WO2010/054406 WO2010/054405, WO2010/054384, WO2012/016184, WO2009/086558, WO2010/042877, WO2011/000106, WO2011/000107, WO2005/120152 WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety. International patent application WO 2019/131770 is also incorporated herein by reference in its entirety.

### b) Stabilizing Lipids (e.g., PEG Lipids)

A stabilizing lipid or surface stabilizing lipid may be used to enhance the structure of the LNP. A stabilizing lipid as contemplated herein may be a polyethylene glycol (PEG)-modified phospholipid.

The use and inclusion of polyethylene glycol (PEG)-modified phospholipids and derivatized lipids such as derivatized ceramides (PEG-CER), including N-Octanoyl-Sphingosine-1-[Succinyl (Methoxy Polyethylene Glycol)-2000](C8 PEG-2000 ceramide) in the liposomal and pharmaceutical compositions described herein is contemplated, preferably in combination with one or more of the compounds and lipids disclosed herein. Contemplated PEG-modified lipids include, but are not limited to, a polyethylene glycol chain of up to 5 kDa in length covalently attached to a lipid with alkyl chain(s) of C6-C20 length. In some embodiments, the PEG-modified lipid employed in the compositions and methods described herein is 1,2-dimyristoyl-sn-glycerol, methoxypolyethylene Glycol (2000 MW PEG) “DMG-PEG2000.” The addition of PEG-modified lipids to the lipid delivery vehicle may prevent complex aggregation and may also provide a means for increasing circulation lifetime and increasing the delivery of the lipid-polynucleotide composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivatized lipids may comprise a molar ratio from about 0% to about 20%, about 0.5% to about 20%, about 1% to about 15%, about 4% to about 10%, or about 2% of the total lipid present in a liposomal lipid nanoparticle.

In some embodiments, the lipid moiety of the PEG-lipids includes those having lengths of from about C14 to about C22, such as from about C14 to about C16. In some embodiments, a PEG moiety, for example a mPEG-NH2, has a size of about 1000, about 2000, about 5000, about 10,000, about 15,000 or about 20,000 daltons

In an embodiment, a PEG-modified lipid is described in International Pat. Appl. No. PCT/US2019/015913, which is incorporated herein by reference in their entirety. In an embodiment, a transfer vehicle comprises one or more PEG-modified lipids.

Non-limiting examples of PEG-modified lipids include PEG-modified phosphatidylethanolamines and phosphatidic acids, PEG-ceramide conjugates (e.g., PEG-CerC14 or PEG-CerC20), PEG-modified dialkylamines and PEG-modified 1,2-diacyloxypropan-3-amines, PEG-modified diacylglycerols, PEG-modified dialkylglycerols, and mixtures thereof. In some further embodiments, a PEG-modified lipid may be, e.g., PEG-c-DOMG, PEG-DMG, PEG-DLPE, PEG-DMPE, PEG-DPPC, PEG-DSPE, PEG-DAG, PEG-S-DAG, PEG-PE, PEG-S-DMG, PEG-CER, PEG-dialkoxypropylcarbamate, PEG-OR, PEG-OH, PEG-c-DOMG, or PEG-1.

In some still further embodiments, the PEG-modified lipid includes, but is not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the lipid nanoparticles described herein can comprise a lipid modified with a non-diffusible PEG. Non-limiting examples of non-diffusible PEGs include PEG-DSG and PEG-DSPE. In one embodiment, the lipid nanoparticles herein comprise PEG-DSPC.

In some embodiments the PEG-modified lipids are a modified form of PEG-DMG. PEG-DMG has the following structure:

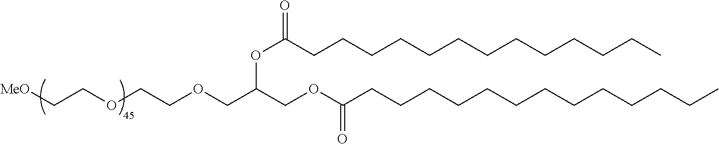

In some embodiments, the PEG lipid is a compound of Formula (PI):

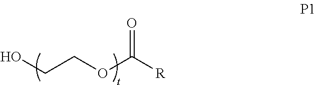

or a salt or isomer thereof, wherein:
r is an integer between 1 and 100;
R is C10-40 alkyl, C10-40 alkenyl, or C10-40 alkynyl; and optionally one or more methylene groups of R are independently replaced with C3-10 carbocyclylene, 4 to 10 membered heterocyclylene, C6-10 arylene, 4 to 10 membered heteroarylene, —N(RN)—, —O—, —S—, —C(O)—, —C(O)N(RN)—, —NRNC(O)—, —NRNC(O)N(RN)—, —C(O)O—, —OC(O)—, —OC(O)O—, —OC(O)N(RN)—, —NRNC(O)O—, —C(O)S—, —SC (O)—, —C(═NRN)—, —C(═NRN)N(RN)—, —NRNC(═NRN)—, —NRNC(═NRN)N(RN)—, —C(S)—, —C(S)N(RN)—, —NRNC(S)—, —NRNC(S)N(RN)—, —S(O)—, —OS (O)—, —S(O)O—, —OS (O)O—, —OS(O)2-, —S(O)2O—, —OS(O)2O—, —N(RN)S(O)—, —S(O)N(RN)—, —N(RN)S(O)N(RN)—, —OS (O)N(RN)—, —N (RN)S(O)O—, —S(0)2-, —N(RN)S(O)2-, —S(O)2N(RN)—, —N(RN)S(O)2N (RN)—, —OS (O)2N(RN)—, or —N(RN)S(O)2O—; and each instance of RN is independently hydrogen, C1-6 alkyl, or a nitrogen protecting group.

For example, R is C17 alkyl. For example, the PEG lipid is a compound of Formula (P1-a):

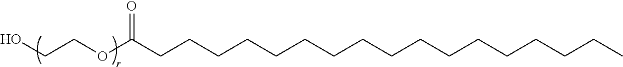

or a salt or isomer thereof, wherein r is an integer between 1 and 100.

In some embodiments the PEG-modified lipids are a modified form of PEG-C18, or PEG-1. PEG-1 has the following structure:

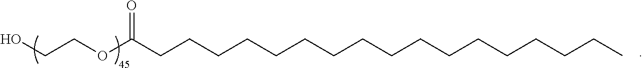

PEG-lipids are known in the art, such as those described in U.S. Pat. No. 8,158,601 and International Pat. Publ. No. WO2015/130584 A2, which are incorporated herein by reference in their entirety. In one embodiment, PEG lipids can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

### c) Helper Lipids

In some embodiments, the transfer vehicle (e.g., LNP) described herein comprises one or more non-cationic helper lipids. In some embodiments, the helper lipid is a phospholipid. In some embodiments, the helper lipid is a phospholipid substitute or replacement. In some embodiments, the phospholipid or phospholipid substitute can be, for example, one or more saturated or (poly) unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the helper lipid is a 1,2-distearoyl-177-glycero-3-phosphocholine (DSPC) analog, a DSPC substitute, oleic acid, or an oleic acid analog.

In some embodiments, a helper lipid is a non-phosphatidyl choline (PC) zwitterionic lipid, a DSPC analog, oleic acid, an oleic acid analog, or a DSPC substitute.

In some embodiments, a helper lipid is described in PCT/US2018/053569. Helper lipids suitable for use in a lipid composition of the disclosure include, for example, a variety of neutral, uncharged or zwitterionic lipids. Such helper lipids are preferably used in combination with one or more of the compounds and lipids disclosed herein. Examples of helper lipids include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoylsn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-paimitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), paimitoyioieoyl phosphatidylcholine (POPC), lysophosphatidyl choline, dioleoyl phosphatidylethanol amine (DOPE)dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the helper lipid may be distearoylphosphatidylcholine (DSPC) or dimyristoyl phosphatidyl ethanolamine (DMPE). In another embodiment, the helper lipid may be distearoylphosphatidylcholine (DSPC). Helper lipids function to stabilize and improve processing of the transfer vehicles. Such helper lipids are preferably used in combination with other excipients, for example, one or more of the ionizable lipids disclosed herein. In some embodiments, when used in combination with an ionizable lipid, the helper lipid may comprise a molar ratio of 5% to about 90%, or about 10% to about 70% of the total lipid present in the lipid nanoparticle.

### d) Structural Lipids

The transfer vehicles described herein comprise one or more structural lipids. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid is an analog of cholesterol. In certain embodiments, the structural lipid includes cholesterol and a corticosteroid (such as, for example, prednisolone, dexamethasone, prednisone, and hydrocortisone), or a combination thereof.

In an embodiment, a structural lipid is described in international patent application PCT/US2019/015913.

In some embodiments, the structural lipid is a sterol (e.g., phytosterols or zoosterols). In certain embodiments, the structural lipid is a steroid. For example, sterols can include, but are not limited to, cholesterol, B-sitosterol, fecosterol, ergosterol, sitosterol, campesterol, stigmasterol, brassicasterol, ergosterol, tomatidine, tomatine, ursolic acid, or alpha-tocopherol. In certain embodiments, the structural lipid is cholesterol. In certain embodiments, the structural lipid is an analog of cholesterol. In certain embodiments, the structural lipid is alpha-tocopherol.

The transfer vehicles described herein comprise one or more structural lipids. Incorporation of structural lipids in a transfer vehicle, e.g., a lipid nanoparticle, may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, a transfer vehicle includes an effective amount of an immune cell delivery potentiating lipid, e.g., a cholesterol analog or an amino lipid or combination thereof, that, when present in a transfer vehicle, e.g., an lipid nanoparticle, may function by enhancing cellular association and/or uptake, internalization, intracellular trafficking and/or processing, and/or endosomal escape and/or may enhance recognition by and/or binding to immune cells, relative to a transfer vehicle lacking the immune cell delivery potentiating lipid. Accordingly, while not intending to be bound by any particular mechanism or theory, in one embodiment, a structural lipid or other immune cell delivery potentiating lipid of the disclosure binds to C1q or promotes the binding of a transfer vehicle comprising such lipid to C1q. Thus, for in vitro use of the transfer vehicles of the disclosure for delivery of a nucleic acid molecule to an immune cell, culture conditions that include C1q are used (e.g., use of culture media that includes serum or addition of exogenous C1q to serum-free media). For in vivo use of the transfer vehicles of the disclosure, the requirement for C1q is supplied by endogenous C1q.

In certain embodiments, the structural lipid is cholesterol. In certain embodiments, the structural lipid is an analog of cholesterol.

### 2. Other Delivery Vehicles Known in the Art

In certain embodiments, other delivery vehicles that are known in the art may be used to transport the circular RNA (i.e., are transfer vehicles encompassed herein).

In some embodiments, liposomes or other lipid bilayer vesicles may be used as a component or as the whole transfer vehicle to facilitate or enhance the delivery and release of circular RAN to one or more target cells. Liposomes are usually characterized by having an interior space sequestered from an outer medium by a membrane of one or more bilayers forming a microscopic sack or vesicle. Bilayer membranes of liposomes are typically formed by lipids, i.e., amphiphilic molecules of synthetic or natural origin that comprise spatially separated hydrophobic or hydrophilic domains (Lasic, D, and Papahadjopoulos, D., eds. Medical Applications of Liposomes. Elsevier, Amsterdam, 1998).

In certain embodiments, the transfer vehicle for transporting the circular RNA comprises a dendrimer. Use of “dendrimer” describes the architectural motif of the transfer vehicle. In some embodiments, the dendrimer includes but is not limited to containing an interior core and one or more layers (i.e., generations) that extend or attach out from the interior core. In some of the embodiments, the generations may contain one or more branching points and an exterior surface of terminal groups that attach to the outermost generation. The branching points, in certain embodiments, may be mostly monodispersed and contain symmetric branching units built around the interior core. In some embodiments, the interior core. Synthesis of the dendrimer may comprise the divergent method, convergent growth, hypercore and branched monomer growth, double exponential growth, lego chemistry, click chemistry and other methods as available in the art (Mendes L. et al., Molecules. 2017. 22 (9): 1401 further describes these methods).

In certain embodiments, as described herein, the transfer vehicle for the circular RNA construct comprises a polymer nanoparticle. In some embodiments, the polymer nanoparticle includes nanocapsules and nanospheres. Nanocapsules, in some embodiments, are composed of an oily core surrounded by a polymeric shell. In some embodiments, the circular RNA is contained within the core and the polymeric shell controls the release of the circular RNA. On the other hand, nanospheres comprise a continuous polymeric network in which the circular RNA is retained or absorbed onto the surface. In some embodiments, cationic polymers are used to encapsulate the circular RNA due to the favorable electrostatic interaction of the cations to the negatively charged nucleic acids and cell membrane. The polymer nanoparticle may be prepared by various methods. In some embodiments, the polymer nanoparticle may be prepared by nanoprecipitation, emulsion techniques, solvent evaporation, solvent diffusion, reverse salting-out or other methods available in the art.

In certain embodiments, as described herein, the transfer vehicle for the circular RNA construct comprises a polymer-lipid hybrid nanoparticle (LPHNP). In some embodiments, the LPHNP comprises a polymer core enveloped within a lipid bilayer. In some embodiments, the polymer core encapsulates the circular RNA construct. In some embodiments, the LPHNP further comprises an outer lipid bilayer. In certain embodiments this outer lipid bilayer comprises a PEG-lipid, helper lipid, cholesterol or other molecule as known in the art to help with stability in a lipid-based nanoparticle. The lipid bilayer closest to the polymer core mitigates the loss of the entrapped circular RNA during LPHNP formation and protects from degradation of the polymer core by preventing diffusion of water from outside of the transfer vehicle into the polymer core (Mukherjee et al., In J. Nanomedicine. 2019; 14:1937-1952).

In certain embodiments, the circular RNA can be transported using a peptide-based delivery mechanism. In some embodiments, the peptide-based delivery mechanism comprises a lipoprotein. Based on the size of the drug to be delivered, the lipoprotein may be either a low-density (LDL) or high-density lipoprotein (HDL). As seen in U.S. Pat. No. 8,734,853B2, high-density lipoproteins are capable of transporting a nucleic acid in vivo and in vitro. In particular embodiments, the lipid component includes cholesterol. In more particular embodiments, the lipid component includes a combination of cholesterol and cholesterol oleate.

In certain embodiments, the circular RNA construct can be transported using a carbohydrate carrier or a sugar-nanocapsule. In certain embodiments, the carbohydrate carrier comprises a sugar-decorated nanoparticle, peptide- and saccharide-conjugated dendrimer, nanoparticles based on polysaccharides, and other carbohydrate-based carriers available in the art. As described herein, the incorporation of carbohydrate molecules may be through synthetic means. In some embodiments, the carbohydrate carrier comprises polysaccharides. These polysaccharides may be made from the microbial cell wall of the target cell. For example, carbohydrate carriers comprised of mannan carbohydrates have been shown to successfully deliver mRNA (Son et al., Nano Lett. 2020. 20 (3): 1499-1509).

In certain embodiments, as provided herein, the transfer vehicle for the circular RNA is a glyconanoparticle (GlycoNP). As known in the art, glyconanoparticles comprise a core comprising gold, iron oxide, semiconductor nanoparticles or a combination thereof. In some embodiments, the glyconanoparticle is functionalized using carbohydrates. In certain embodiments, the glyconanoparticle comprises a carbon nanotube or graphene. In one embodiment the glyconanoparticle comprises a polysaccharide-based GlycoNP (e.g., chitosan-based GlycoNP). In certain embodiments, the glyconanoparticle is a glycodendrimer.

In certain embodiments, as provided herein, the circular RNA is transferred through use of an exosome, a type of extracellular vesicle. Exosomes naturally are secreted by various types of cells and are used as a transport vesicle for various forms of cargo. During delivery exosomes can contain and protect specific mRNAs, regulatory microRNAs, lipids, and proteins (Luan et al., Acta Pharmacologica Sinica. 2017. 38:754-763). Naturally, exosomes may be 30 nm to 125 nm.

In some embodiments, the exosome may be made in part from an immune cell. As shown in Haney et al, use of immune cell derived exosomes are able to avoid mononuclear phagocytes (J Control Release. 2015. 207:18-30). In some embodiments, the exosome may be a dendritic cell, macrophage, T-cell, B-cell or derived from another immune cell. As seen in WO/2021/041473A1, various forms of RNAs of varying lengths may be transported through exosome delivery including messenger RNA (mRNA), microRNA (miRNA), long intergenic non-coding RNA (lincRNA), long non-coding RNA (lncRNA), non-coding RNA (ncRNA), non-messenger RNA (nmRNA), small RNA (sRNA), small non-messenger RNA (smnRNA), DNA damage response RNA (DD RNA), extracellular RNA (exRNA), small nuclear RNA (snRNA), small nucleolar RNA (snoRNA), and precursor messenger RNA (pre-mRNA).

In other embodiments, the transfer vehicle may comprise in whole or in part from a fusome. In some embodiments, the fusome is derived from an endoplasmic reticulum of a germline cyst. In certain embodiments, the germline cyst is from a Drosophila ovary.

In certain embodiments, the circular RNA construct may be transported using noncellular and instead be through mechanical delivery mechanisms. In some embodiments, this delivery method includes microneedles, electroporation, continuous pumps and/or gene guns.

In some embodiments, the transfer vehicle of the circular RNA construct is a solution or diluent comprising of a salt or a buffer.

### 3. Targeting

In some embodiments, the compositions use targeting moieties that may be bound (either covalently or non-covalently) to the transfer vehicle to encourage localization of such transfer vehicle at certain target cells or target tissues. For example, targeting may be mediated by the inclusion of one or more endogenous targeting moieties in or on the transfer vehicle to encourage distribution to the target cells or tissues. Recognition of the targeting moiety by the target tissues actively facilitates tissue distribution and cellular uptake of the transfer vehicle and/or its contents in the target cells and tissues (e.g., the inclusion of an apolipoprotein-E targeting ligand in or on the transfer vehicle encourages recognition and binding of the transfer vehicle to endogenous low density lipoprotein receptors expressed by hepatocytes).

As provided herein, the composition can comprise a moiety capable of enhancing affinity of the composition to the target cell. Targeting moieties may be linked to the outer bilayer of the lipid particle during formulation or post-formulation. These methods are well known in the art. In addition, some lipid particle formulations may employ fusogenic polymers such as PEAA, hemagluttinin, other lipopeptides (see U.S. patent application Ser. No. 08/835,281, and 60/083,294, which are incorporated herein by reference) and other features useful for in vivo and/or intracellular delivery. In other some embodiments, the compositions demonstrate improved transfection efficacies, and/or demonstrate enhanced selectivity towards target cells or tissues of interest. Contemplated therefore are compositions which comprise one or more moieties (e.g., peptides, aptamers, oligonucleotides, a vitamin or other molecules) that are capable of enhancing the affinity of the compositions and their nucleic acid contents for the target cells or tissues. Suitable moieties may optionally be bound or linked to the surface of the transfer vehicle. In some embodiments, the targeting moiety may span the surface of a transfer vehicle or be encapsulated within the transfer vehicle. Suitable moieties and are selected based upon their physical, chemical or biological properties (e.g., selective affinity and/or recognition of target cell surface markers or features). Cell-specific target sites and their corresponding targeting ligand can vary widely. Suitable targeting moieties are selected such that the unique characteristics of a target cell are exploited, thus allowing the composition to discriminate between target and non-target cells. For example, in some embodiments, compositions may include surface markers (e.g., apolipoprotein-B or apolipoprotein-E) that selectively enhance recognition of, or affinity to hepatocytes (e.g., by receptor-mediated recognition of and binding to such surface markers). As an example, the use of galactose as a targeting moiety would be expected to direct the compositions to parenchymal hepatocytes, or alternatively the use of mannose containing sugar residues as a targeting ligand would be expected to direct the compositions to liver endothelial cells (e.g., mannose containing sugar residues that may bind preferentially to the asialoglycoprotein receptor present in hepatocytes). (See Hillery A M, et al. “Drug Delivery and Targeting: For Pharmacists and Pharmaceutical Scientists” (2002) Taylor & Francis, Inc.) The presentation of such targeting moieties that have been conjugated to moieties present in the transfer vehicle (e.g., a lipid nanoparticle) therefore facilitate recognition and uptake of the compositions in target cells and tissues. Examples of suitable targeting moieties include one or more peptides, proteins, aptamers, vitamins and oligonucleotides.

In particular embodiments, a transfer vehicle comprises a targeting moiety. In some embodiments, the targeting moiety mediates receptor-mediated endocytosis selectively into a specific population of cells. In some embodiments, the targeting moiety is capable of binding to a T cell antigen. In some embodiments, the targeting moiety is capable of binding to a NK, NKT, or macrophage antigen. In some embodiments, the targeting moiety is capable of binding to a protein selected from the group CD3, CD4, CD8, PD-1, 4-1BB, and CD2. In some embodiments, the targeting moiety is a single chain Fv(scFv) fragment, nanobody, peptide, peptide-based macrocycle, minibody, heavy chain variable region, light chain variable region or fragment thereof. In some embodiments, the targeting moiety is selected from T-cell receptor motif antibodies, T-cell a chain antibodies, T-cell β chain antibodies, T-cell y chain antibodies, T-cell δ chain antibodies, CCR7 antibodies, CD3 antibodies, CD4 antibodies, CD5 antibodies, CD7 antibodies, CD8 antibodies, CD11b antibodies, CD11c antibodies, CD16 antibodies, CD19 antibodies, CD20 antibodies, CD21 antibodies, CD22 antibodies, CD25 antibodies, CD28 antibodies, CD34 antibodies, CD35 antibodies, CD40 antibodies, CD45RA antibodies, CD45RO antibodies, CD52 antibodies, CD56 antibodies, CD62L antibodies, CD68 antibodies, CD80 antibodies, CD95 antibodies, CD117 antibodies, CD127 antibodies, CD133 antibodies, CD137 (4-1BB) antibodies, CD163 antibodies, F4/80 antibodies, IL-4Rα antibodies, Sca-1 antibodies, CTLA-4 antibodies, GITR antibodies GARP antibodies, LAP antibodies, granzyme B antibodies, LFA-1 antibodies, transferrin receptor antibodies, and fragments thereof. In some embodiments, the targeting moiety is a small molecule binder of an ectoenzyme on lymphocytes. Small molecule binders of ectoenzymes include A2A inhibitors CD73 inhibitors, CD39 or adesines receptors A2aR and A2bR. Potential small molecules include AB928.

In some embodiments, transfer vehicles are formulated and/or targeted as described in Shobaki N, Sato Y, Harashima H. Mixing lipids to manipulate the ionization status of lipid nanoparticles for specific tissue targeting. Int J Nanomedicine. 2018; 13:8395-8410. Published 2018 Dec. 10. In some embodiments, a transfer vehicle is made up of 3 lipid types. In some embodiments, a transfer vehicle is made up of 4 lipid types. In some embodiments, a transfer vehicle is made up of 5 lipid types. In some embodiments, a transfer vehicle is made up of 6 lipid types.

### F. Pharmaceutical Compositions

Provided herein is a pharmaceutical composition comprising at least one circular RNA construct and a transfer vehicle. Also provided herein is a pharmaceutical composition comprising at least one circular RNA construct described herein and a pharmaceutically acceptable excipient. In some embodiments, and as described elsewhere herein, the pharmaceutical composition comprises at least one circular RNA construct and a transfer vehicle comprising an ionizable lipid. In some embodiments, the pharmaceutical composition comprises at least one circular RNA construct and a transfer vehicle, where the transfer vehicle is a nanoparticle or lipid nanoparticle.

In some embodiments, the pharmaceutical composition comprises at least one circular RNA construct, a nanoparticle, and optionally, a targeting moiety operably connected to the nanoparticle. In some embodiments, the nanoparticle is a lipid nanoparticle (LNP), a core-shell nanoparticle, a biodegradable nanoparticle, a biodegradable lipid nanoparticle, a polymer nanoparticle, a polyplex or a biodegradable polymer nanoparticle. In some embodiments, the pharmaceutical composition comprises a targeting moiety, wherein the targeting moiety mediates receptor-mediated endocytosis, endosome fusion, or direct fusion into selected cells of a selected cell population or tissue in the absence of cell isolation or purification. In some embodiments, the pharmaceutical composition comprises a targeting moiety operably connected to the nanoparticle. In some embodiments, the targeting moiety is a small molecule, scFv, nanobody, peptide, cyclic peptide, di or tri cyclic peptide, minibody, polynucleotide aptamer, engineered scaffold protein, heavy chain variable region, light chain variable region, or a fragment thereof. In some embodiments, less than 1%, by weight, of the polynucleotides in the composition are double stranded RNA, DNA splints, DNA template, or triphosphorylated RNA. In some embodiments, less than 1%, by weight, of the polynucleotides and proteins in the pharmaceutical composition are double stranded RNA, DNA splints, DNA template, triphosphorylated RNA, phosphatase proteins, protein ligases, RNA polymerases, and capping enzymes.

Also provided herein is a pharmaceutical composition comprising a circular RNA construct and a pharmaceutical salt, buffer, diluent or combination thereof.

In certain embodiments, provided herein are compositions (e.g., pharmaceutical compositions) comprising a therapeutic agent provided herein. In some embodiments, the therapeutic agent is a circular RNA polynucleotide provided herein. In some embodiments the therapeutic agent is a vector provided herein. In some embodiments, the therapeutic agent is a cell comprising a circular RNA or vector provided herein (e.g., a human cell, such as a human T cell). In certain embodiments, the composition further comprises a pharmaceutically acceptable carrier. In some embodiments, the compositions provided herein comprise a therapeutic agent provided herein in combination with other pharmaceutically active agents or drugs, such as anti-inflammatory drugs or antibodies capable of targeting B cell antigens, e.g., anti-CD20 antibodies, e.g., rituximab.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the therapeutic agent(s) and one which has no detrimental side effects or toxicity under the conditions of use.

The choice of carrier will be determined in part by the particular therapeutic agent, as well as by the particular method used to administer the therapeutic agent. Accordingly, there are a variety of suitable formulations of the pharmaceutical compositions known in the art. In certain embodiments, the pharmaceutical composition comprises a preservative. In some embodiments, the pharmaceutical composition comprises a buffering agent.

In some embodiments, the concentration of therapeutic agent in the pharmaceutical composition can vary, e.g., less than about 1%, or at least about 1%, 2%, 3%, 4%, 5%, 6%, 7%, 8%, 9% 10%, 15%, 20%, 25%, 30%, 35%, 40%, 45%, or about 50% or more by weight, and can be selected primarily by fluid volumes, and viscosities, in accordance with the particular mode of administration selected.

Formulations for oral, aerosol, parenteral (e.g., subcutaneous, intravenous, intraarterial, intramuscular, intradermal, intraperitoneal, and intrathecal), and topical administration are known in the art. More than one route can be used to administer the therapeutic agents provided herein, and in certain instances, a particular route can provide a more immediate and more effective response than another route.

In certain embodiments, the therapeutic agents provided herein can be formulated as inclusion complexes, such as cyclodextrin inclusion complexes, or LNPs or liposomes.

In some embodiments, the therapeutic agents provided herein are formulated in time-released, delayed release, or sustained release delivery systems such that the delivery of the composition occurs prior to, and with sufficient time to, cause sensitization of the site to be treated. Such systems can avoid repeated administrations of the therapeutic agent, thereby increasing convenience to the subject and the physician, and may be particularly suitable for certain composition embodiments provided herein. In one embodiment, the compositions are formulated such that they are suitable for extended-release of the circular RNA contained therein. Such extended-release compositions may be conveniently administered to a subject at extended dosing intervals. For example, in one embodiment, the compositions are administered to a subject twice a day, daily or every other day. In an embodiment, the compositions are administered to a subject twice a week, once a week, every ten days, every two weeks, every three weeks, every four weeks, once a month, every six weeks, every eight weeks, every three months, every four months, every six months, every eight months, every nine months or annually.

In some embodiments, a protein encoded by an inventive polynucleotide is produced by a target cell for sustained amounts of time. For example, the protein may be produced for more than one hour, more than four, more than six, more than 12, more than 24, more than 48 hours, or more than 72 hours after administration. In some embodiments the polypeptide is expressed at a peak level about six hours after administration. In some embodiments the expression of the polypeptide is sustained at least at a therapeutic level. In some embodiments, the polypeptide is expressed at least at a therapeutic level for more than one, more than four, more than six, more than 12, more than 24, more than 48, or more than 72 hours after administration. In some embodiments, the polypeptide is detectable at a therapeutic level in patient tissue (e.g., liver or lung). In some embodiments, the level of detectable polypeptide is from continuous expression from the circular RNA composition over periods of time of more than one, more than four, more than six, more than 12, more than 24, more than 48, or more than 72 hours after administration.

In some embodiments, the circular RNA constructs disclosed herein (optionally with the transfer vehicles disclosed herein) are administered to a subject twice a day, daily or every other day. In an embodiment, the compositions are administered to a subject twice a week, once a week, every ten days, every two weeks, every three weeks, every four weeks, once a month, every six weeks, every eight weeks, every three months, every four months, every six months, every eight months, every nine months or annually.

In some embodiments, the circular RNA constructs disclosed herein (optionally with the transfer vehicles disclosed herein) are administered every other day. In some embodiments, the circular RNA constructs disclosed herein (optionally with the transfer vehicles disclosed herein) are administered once a week (i.e., weekly). In some embodiments, the circular RNA constructs disclosed herein (optionally with the transfer vehicles disclosed herein) are administered every two weeks (i.e., every other week or biweekly, q2w). In some embodiments, administration of the circular RNA constructs disclosed herein (optionally with the transfer vehicles disclosed herein) occurs less frequently and/or at a lower dose than as required for a suitable comparator, for example, a corresponding linear RNA.

In certain embodiments, a protein encoded by an inventive polynucleotide is produced at levels above normal physiological levels. The level of protein may be increased as compared to a control. In some embodiments, the control is the baseline physiological level of the polypeptide in a normal individual or in a population of normal individuals. In other embodiments, the control is the baseline physiological level of the polypeptide in an individual having a deficiency in the relevant protein or polypeptide or in a population of individuals having a deficiency in the relevant protein or polypeptide. In some embodiments, the control can be the normal level of the relevant protein or polypeptide in the individual to whom the composition is administered. In other embodiments, the control is the expression level of the polypeptide upon other therapeutic intervention, e.g., upon direct injection of the corresponding polypeptide, at one or more comparable time points.

In certain embodiments, the levels of a protein encoded by an inventive polynucleotide are detectable at 3 days, 4 days, 5 days, or 1 week or more after administration. Increased levels of protein may be observed in a tissue (e.g., liver or lung).

In some embodiments, the method yields a sustained circulation half-life of a protein encoded by an inventive polynucleotide. For example, the protein may be detected for hours or days longer than the half-life observed via subcutaneous injection of the protein or mRNA encoding the protein. In some embodiments, the half-life of the protein is 1 day, 2 days, 3 days, 4 days, 5 days, or 1 week or more.

Different types of release delivery systems are available for the compositions and known to those of ordinary skill in the art.

In certain embodiments, the compositions may be loaded with diagnostic radionuclide, fluorescent materials or other materials that are detectable in both in vitro and in vivo applications. For example, suitable diagnostic materials may include Rhodamine-dioleoylphosphatidylethanolamine (Rh-PE), Green Fluorescent Protein circRNA (GFP circRNA), Renilla Luciferase circRNA and Firefly Luciferase circRNA.

### I. Methods

### A. Method of Preparing

### 1. Precursor RNA Preparation

Transcription of a DNA template (e.g., comprising a 3′ enhanced intron element, 3′ enhanced exon element, a core functional element including an IRES and expression sequence, a 5′ enhanced exon element, and a 5′ enhanced intron element) results in formation of a precursor linear RNA polynucleotide capable of circularizing. In some embodiments, this DNA template comprises a vector, PCR product, plasmid, minicircle DNA, cosmid, artificial chromosome, complementary DNA (cDNA), extrachromosomal DNA (ecDNA), or a fragment therein. In certain embodiments, the minicircle DNA may be linearized or non-linearized. In certain embodiments, the plasmid may be linearized or non-linearized. In some embodiments, the DNA template may be single-stranded. In other embodiments, the DNA template may be double-stranded. In some embodiments, the DNA template comprises in whole or in part from a viral, bacterial or eukaryotic vector.

In some embodiments, the DNA template shares the same sequence as the precursor linear RNA polynucleotide prior to splicing of the precursor linear RNA polynucleotide (e.g., a 3′ enhanced intron element, a 3′ enhanced exon element, a core functional element, and a 5′ enhanced exon element, a 5′ enhanced intron element). In some embodiments, said linear precursor RNA polynucleotide undergoes splicing leading to the removal of the 3′ enhanced intron element and 5′ enhanced intron element during the process of circularization. In some embodiments, the resulting circular RNA polynucleotide lacks a 3′ enhanced intron fragment and a 5′ enhanced intron fragment, but maintains a 3′ enhanced exon fragment, a core functional element, and a 5′ enhanced exon element.

In some embodiments, the precursor linear RNA polynucleotide circularizes when incubated in the presence of one or more guanosine nucleotides or nucleoside (e.g., GTP) and a divalent cation (e.g., Mg2+). In some embodiments, the 3′ enhanced exon element, 5′ enhanced exon element, and/or core functional element in whole or in part promotes the circularization of the precursor linear RNA polynucleotide to form the circular RNA construct provided herein.

In certain embodiments, circular RNA provided herein is produced inside a cell. In some embodiments, precursor RNA is transcribed using a DNA template (e.g., in some embodiments, using a vector provided herein) in the cytoplasm by a bacteriophage RNA polymerase, or in the nucleus by host RNA polymerase II and then circularized.

The precursor RNA provided herein can be generated by incubating a DNA template provided herein under conditions permissive of transcription of the precursor RNA encoded by the DNA template. For example, in some embodiments a precursor RNA is synthesized by incubating a DNA template provided herein that comprises an RNA polymerase promoter upstream of its 5′ duplex sequence and/or expression sequences with a compatible RNA polymerase enzyme under conditions permissive of in vitro transcription. In some embodiments, the DNA template is incubated inside of a cell by a bacteriophage RNA polymerase or in the nucleus of a cell by host RNA polymerase II.

In certain embodiments, provided herein is a method of generating precursor RNA by performing in vitro transcription using a DNA template provided herein as a template (e.g., a vector provided herein with an RNA polymerase promoter positioned upstream of the 5′ duplex region).

In certain embodiments, the resulting precursor RNA can be used to generate circular RNA (e.g., a circular RNA construct provided herein) by incubating it in the presence of magnesium ions and guanosine nucleotide or nucleoside at a temperature at which RNA circularization occurs (e.g., between 20° C. and 60° C.). Precursor RNA are generally described in WO2022/261490, which is incorporated herein by reference in its entirety.

### 2. Circular RNA Preparation

Also provided herein is a method for preparing or generating circular RNA. In certain embodiments, the method comprises synthesizing precursor RNA by transcription (e.g., run-off transcription) using a vector as a template, and incubating the resulting precursor RNA in the presence of divalent cations (e.g., magnesium ions) and GTP such that it circularizes to form circular RNA. In some embodiments, an inventive precursor RNA is capable of circularizing in the absence of magnesium ions and GTP and/or without the step of incubation with magnesium ions and GTP. In some embodiments, transcription is carried out in the presence of an excess of GMP.

Thus, in certain embodiments provided herein is a method of making circular RNA. In certain embodiments, the method comprises synthesizing precursor RNA by transcription (e.g., run-off transcription) using a vector provided herein (e.g., a 5′ enhanced intron element, a 5′ enhanced exon element, a core functional element, a 3′ enhanced exon element, and a 3′ enhanced intron element) as a template, and incubating the resulting precursor RNA in the presence of divalent cations (e.g., magnesium ions) and GTP such that it circularizes to form circular RNA. In some embodiments, the precursor RNA disclosed herein is capable of circularizing in the absence of magnesium ions and GTP and/or without the step of incubation with magnesium ions and GTP. It has been discovered that circular RNA has reduced immunogenicity relative to a corresponding mRNA, at least partially because the mRNA contains an immunogenic 5′ cap. When transcribing a DNA vector from certain promoters (e.g., a T7 promoter) to produce a precursor RNA, it is understood that the 5′ end of the precursor RNA is G. To reduce the immunogenicity of a circular RNA composition that contains a low level of contaminant linear mRNA, an excess of GMP relative to GTP can be provided during transcription such that most transcripts contain a 5′ GMP, which cannot be capped. Therefore, in some embodiments, transcription is carried out in the presence of an excess of GMP. In some embodiments, transcription is carried out where the ratio of GMP concentration to GTP concentration is within the range of about 3:1 to about 15:1, for example, about 3:1 to about 10:1, about 3:1 to about 5:1, about 3:1, about 4:1, or about 5:1.

In some embodiments, a composition comprising circular RNA has been purified. Circular RNA may be purified by any known method commonly used in the art, such as column chromatography, gel filtration chromatography, and size exclusion chromatography. In some embodiments, purification comprises one or more of the following steps: phosphatase treatment, HPLC size exclusion purification, and RNase R digestion. In some embodiments, purification comprises the following steps in order: RNase R digestion, phosphatase treatment, and HPLC size exclusion purification. In some embodiments, purification comprises reverse phase HPLC. In some embodiments, a purified composition contains less double stranded RNA, DNA splints, triphosphorylated RNA, phosphatase proteins, protein ligases, capping enzymes and/or nicked RNA than unpurified RNA. In some embodiments, purification of circular RNA comprises an affinity-purification or negative selection method described herein. In some embodiments, purification of circular RNA comprises separation of linear RNA from circular RNA using oligonucleotides that are complementary to a sequence in the linear RNA but are not complementary to a sequence in the circular RNA. In some embodiments, a purified composition is less immunogenic than an unpurified composition. In some embodiments, immune cells exposed to a purified composition produce less TNFα, RIG-I, IL-2, IL-6, IFNγ, and/or a type 1 interferon, e.g., IFN-β1, than immune cells exposed to an unpurified composition.

Exemplary methods of circularization of precursor RNA can be found in, for example, WO2020/237227, which is incorporated by reference herein in its entirety. WO2020/237227, inter alia, describes using the permuted intron exon (PIE) circularization strategy to circularize long precursor RNA. In it, a 1.1 kb sequence containing a full-length encephalomyocarditis virus (EMCV) IRES, a Gaussia luciferase (GLuc) expression sequence, and two short exon fragments of the permuted intron-exon (PIE) construct were inserted between the 3′ and 5′ introns of the permuted group I catalytic intron in the thymidylate synthase (Td) gene of the T4 phage. Precursor RNA was synthesized by run-off transcription. Circularization was attempted by heating the precursor RNA in the presence of magnesium ions and GTP, but splicing products were not obtained. Perfectly complementary 9 nucleotide and 19 nucleotide long homology regions were designed and added at the 5′ and 3′ ends of the precursor RNA. The splicing product was treated with RNase R. Sequencing across the putative splice junction of RNase R-treated splicing reactions revealed ligated exons, and digestion of the RNase R-treated splicing reaction with oligonucleotide-targeted RNase H produced a single band in contrast to two bands yielded by RNase H-digested linear precursor. WO2020/237227 further indicates that a series of spacers was designed and inserted between the 3′ PIE splice site and the IRES. These spacers were designed to either conserve or disrupt secondary structures within intron sequences in the IRES, 3′ PIE splice site, and/or 5′ splice site.

Further methods for preparing circular RNA are described in WO2022/261490, which is incorporated herein by reference in its entirety.

### 3. Lipid Nanoparticle Preparation

In one embodiment, a lipid nanoparticle formulation may be prepared by the methods described in International Publication Nos. WO2011127255 or WO2008103276, each of which is herein incorporated by reference in their entirety. In some embodiments, lipid nanoparticle formulations may be as described in International Publication No. WO2019131770, which is herein incorporated by reference in its entirety.

In some embodiments, circular RNA is formulated according to a process described in U.S. patent application Ser. No. 15/809,680. In some embodiments, the present disclosure provides a process of encapsulating circular RNA in transfer vehicles comprising the steps of forming lipids into pre-formed transfer vehicles (i.e., formed in the absence of RNA) and then combining the pre-formed transfer vehicles with RNA. In some embodiments, the novel formulation process results in an RNA formulation with higher potency (peptide or protein expression) and higher efficacy (improvement of a biologically relevant endpoint) both in vitro and in vivo with potentially better tolerability as compared to the same RNA formulation prepared without the step of preforming the lipid nanoparticles (e.g., combining the lipids directly with the RNA).

For certain cationic lipid nanoparticle formulations of RNA, in order to achieve high encapsulation of RNA, the RNA in buffer (e.g., citrate buffer) has to be heated. In those processes or methods, the heating is required to occur before the formulation process (i.e., heating the separate components) as heating post-formulation (post-formation of nanoparticles) does not increase the encapsulation efficiency of the RNA in the lipid nanoparticles. In contrast, in some embodiments, the order of heating of RNA does not appear to affect the RNA encapsulation percentage. In some embodiments, no heating (i.e., maintaining at ambient temperature) of one or more of the solutions comprising the pre-formed lipid nanoparticles, the solution comprising the RNA and the mixed solution comprising the lipid nanoparticle encapsulated RNA is required to occur before or after the formulation process.

RNA may be provided in a solution to be mixed with a lipid solution such that the RNA may be encapsulated in lipid nanoparticles. A suitable RNA solution may be any aqueous solution containing RNA to be encapsulated at various concentrations. For example, a suitable RNA solution may contain an RNA at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, or 1.0 mg/ml. In some embodiments, a suitable RNA solution may contain an RNA at a concentration in a range from about 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml.

Typically, a suitable RNA solution may also contain a buffering agent and/or salt. Generally, buffering agents can include HEPES, Tris, ammonium sulfate, sodium bicarbonate, sodium citrate, sodium acetate, potassium phosphate or sodium phosphate. In some embodiments, suitable concentration of the buffering agent may be in a range from about 0.1 mM to 100 mM, 0.5 mM to 90 mM, 1.0 mM to 80 mM, 2 mM to 70 mM, 3 mM to 60 mM, 4 mM to 50 mM, 5 mM to 40 mM, 6 mM to 30 mM, 7 mM to 20 mM, 8 mM to 15 mM, or 9 to 12 mM.

Exemplary salts can include sodium chloride, magnesium chloride, and potassium chloride. In some embodiments, suitable concentration of salts in an RNA solution may be in a range from about 1 mM to 500 mM, 5 mM to 400 mM, 10 mM to 350 mM, 15 mM to 300 mM, 20 mM to 250 mM, 30 mM to 200 mM, 40 mM to 190 mM, 50 mM to 180 mM, 50 mM to 170 mM, 50 mM to 160 mM, 50 mM to 150 mM, or 50 mM to 100 mM.

In some embodiments, a suitable RNA solution may have a pH in a range from about 3.5-6.5, 3.5-6.0, 3.5-5.5, 3.5-5.0, 3.5-4.5, 4.0-5.5, 4.0-5.0, 4.0-4.9, 4.0-4.8, 4.0-4.7, 4.0-4.6, or 4.0-4.5.

Various methods may be used to prepare a suitable RNA solution. In some embodiments, RNA may be directly dissolved in a buffer solution described herein. In some embodiments, an RNA solution may be generated by mixing an RNA stock solution with a buffer solution prior to mixing with a lipid solution for encapsulation. In some embodiments, an RNA solution may be generated by mixing an RNA stock solution with a buffer solution immediately before mixing with a lipid solution for encapsulation.

According to the present disclosure, a lipid solution contains a mixture of lipids suitable to form transfer vehicles for encapsulation of RNA. In some embodiments, a suitable lipid solution is ethanol based. For example, a suitable lipid solution may contain a mixture of desired lipids dissolved in pure ethanol (i.e., 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

A suitable lipid solution may contain a mixture of desired lipids at various concentrations. In some embodiments, a suitable lipid solution may contain a mixture of desired lipids at a total concentration in a range from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml.

Nanoparticles can be made in a 1 fluid stream or with mixing processes such as microfluidics and T-junction mixing of two fluid streams, one of which contains the circular RNA and the other has the lipid components. Exemplary lipid compositions can be prepared according to the methods and at the ratios described in Example 1 and Tables 4a-b.

In some embodiments, the lipid nanoparticles described herein may be synthesized using methods comprising, for example, microfluidic mixers, microstructure-induced chaotic advection (MICA), a Slit Interdigital Microstructured Mixer (SIMM-V2) or a Standard Slit Interdigital Micro Mixer (SSIMM) or Caterpillar (CPMM) or Impinging-jet (IJMM) from the Institut fur Mikrotechnik Mainz GmbH, Mainz Germany), using a micromixer chip, and/or using technology. Exemplary mixers and methods are known in the art.

Additional lipid nanoparticle formulations and methods of producing are described in detail in WO2021226597 and WO2021113777, which are incorporated herein by reference in their entireties.

For example, disclosed in WO2021226597 and WO2021113777 is a method of preparing lipid nanoparticle formulations of ionizable lipids 128 and 129 of Table 3. Ethanol phase contained ionizable Lipid 128 or Lipid 129 from Table 3, DOPE, Cholesterol, and DSPE-PEG 2000 (Avanti Polar Lipids Inc.) at a weight ratio of 16:1:4:1 or 62:4:33:1 molar ratio combined with an aqueous phase containing circular RNA and 25 mM sodium acetate buffer at pH 5.2. A 3:1 aqueous to ethanol mixing ratio was used. The formulated LNPs were then dialyzed in 1L of water and exchanged 2 times over 18 hours. Dialyzed LNPs were filtered using 0.2 μm filter. Prior to in vivo dosing, LNPs were diluted in PBS. LNP sizes were determined by dynamic light scattering. A cuvette with 1 mL of 20 μg/mL LNPs in PBS (pH 7.4) was measured for Z-average using the Malvern Panalytical Zetasizer Pro. The Z-average and polydispersity index were recorded.

Additional exemplary methods for preparing nanoparticle compositions and synthesis of certain ionizable lipids, e.g., for use in the lipid nanoparticles, can be found in Examples 1 and 2.

### B. Method of Treating

Also provided herein is a method of treating a subject in need thereof comprising administering a therapeutically effective amount of the circular RNA construct provided herein. In some embodiments, provided herein is a method of treating a subject in need thereof comprising administering a therapeutically effective amount of a composition comprising the circular RNA construct provided herein. In some embodiments, in addition to the circular RNA construct, a delivery vehicle, and optionally, a targeting moiety operably connected to the delivery vehicle is administered.

Thus, in certain embodiments, provided herein are methods of treating and/or preventing a disease in a subject (e.g., mammalian subject, such as a human subject). Without being bound to a particular theory or mechanism, where the circular RNA encodes a CAR, the CARs have biological activity, e.g., ability to recognize an antigen, e.g., CD19, HER2, or BCMA, such that the CAR, when expressed by a cell, is able to mediate an immune response against the cell expressing the antigen, e.g., CD19, HER2, or BCMA, for which the CAR is specific. In this regard, an embodiment provided herein provides a method of treating or preventing cancer in a subject, comprising administering to the subject the circular RNA therapeutic agents, and/or the pharmaceutical compositions provided herein in an amount effective to treat or prevent cancer in the subject.

In some embodiments, the subject has a cancer selected from the group consisting of: acute myeloid leukemia (AML); alveolar rhabdomyosarcoma; B cell malignancies; bladder cancer (e.g., bladder carcinoma); bone cancer; brain cancer (e.g., medulloblastoma and glioblastoma multiforme); breast cancer; cancer of the anus, anal canal, or anorectum; cancer of the eye; cancer of the intrahepatic bile duct; cancer of the joints; cancer of the neck; gallbladder cancer; cancer of the pleura; cancer of the nose, nasal cavity, or middle ear; cancer of the oral cavity; cancer of the vulva; chronic lymphocytic leukemia; chronic myeloid cancer; colon cancer; esophageal cancer, cervical cancer; fibrosarcoma; gastrointestinal carcinoid tumor; head and neck cancer (e.g., head and neck squamous cell carcinoma); Hodgkin lymphoma; hypopharynx cancer; kidney cancer; larynx cancer; leukemia; liquid tumors; lipoma; liver cancer; lung cancer (e.g., non-small cell lung carcinoma, lung adenocarcinoma, and small cell lung carcinoma); lymphoma; mesothelioma; mastocytoma; melanoma; multiple myeloma; nasopharynx cancer; non-Hodgkin lymphoma; B-chronic lymphocytic leukemia; hairy cell leukemia; Burkitt's lymphoma; ovarian cancer; pancreatic cancer; cancer of the peritoneum; cancer of the omentum; mesentery cancer; pharynx cancer; prostate cancer; rectal cancer; renal cancer; skin cancer; small intestine cancer; soft tissue cancer; solid tumors; synovial sarcoma; gastric cancer; teratoma; testicular cancer; thyroid cancer; and ureter cancer.

In some embodiments, the subject has an autoimmune disease or disorder.

In some embodiments, the subject has an autoimmune disease or disorder selected from scleroderma, Grave's disease, Crohn's disease, Sjogren's disease, multiple sclerosis, Hashimoto's disease, psoriasis, myasthenia gravis, autoimmune polyendocrinopathy syndromes, Type I diabetes mellitus (TIDM), autoimmune gastritis, autoimmune uveoretinitis, polymyositis, colitis, thyroiditis, and the generalized autoimmune diseases typified by human Lupus.

In some embodiments, the subject has an autoimmune disease or disorder that is B-cell mediated.

In some embodiments, the subject has lupus, e.g., systemic lupus erythematosus (SLE), cutaneous lupus erythematosus (CLE), lupus nephritis (LN), neonatal lupus, drug-induced lupus.

In the subject has antisynthetase syndrome, multifocal motor neuropathy, myasthenia gravis, neuromyelitis optica, pemphigus vulgaris, and/or systemic sclerosis.

In some embodiments, provided herein is a method of treating and/or preventing cancer in a subject, comprising introducing the circular RNA and/or pharmaceutical composition thereof disclosed herein. In some embodiments, provided herein is a method of treating cancer in a subject, comprising introducing the circular RNA encoding CD19 CAR and/or pharmaceutical composition thereof disclosed herein. In some embodiments, provided herein is a method of treating cancer in a subject, comprising introducing the circular RNA encoding HER2 CAR and/or pharmaceutical composition thereof disclosed herein. In some embodiments, provided herein is a method of treating cancer in a subject, comprising introducing the circular RNA encoding BCMA CAR and/or pharmaceutical composition thereof disclosed herein.

In some embodiments, provided herein is a method of treating and/or preventing B-cell lymphoma, e.g., large B-cell lymphoma, in a subject, comprising introducing the circular RNA and/or pharmaceutical composition thereof disclosed herein. In some embodiments, provided herein is a method of treating B-cell lymphoma, e.g., large B-cell lymphoma, in a subject, comprising introducing the circular RNA encoding CD19 CAR and/or pharmaceutical composition thereof disclosed herein. In some embodiments, provided herein is a method of treating B-cell lymphoma, e.g., large B-cell lymphoma, in a subject, comprising introducing the circular RNA encoding CD19 CAR and comprising an IRES and/or pharmaceutical composition thereof disclosed herein.

In some embodiments, provided herein is a method of treating and/or preventing mantle cell lymphoma in a subject, comprising introducing the circular RNA and/or pharmaceutical composition thereof disclosed herein. In some embodiments, provided herein is a method of treating mantle cell lymphoma in a subject, comprising introducing the circular RNA encoding CD19 CAR and/or pharmaceutical composition thereof disclosed herein. In some embodiments, provided herein is a method of treating mantle cell lymphoma in a subject, comprising introducing the circular RNA encoding CD19 CAR and comprising an IRES and/or pharmaceutical composition thereof disclosed herein.

In some embodiments, provided herein is a method of treating and/or preventing multiple myeloma in a subject, comprising introducing the circular RNA and/or pharmaceutical composition thereof disclosed herein. In some embodiments, provided herein is a method of treating multiple myeloma in a subject, comprising introducing the circular RNA encoding BCMA CAR and/or pharmaceutical composition thereof disclosed herein. In some embodiments, provided herein is a method of treating multiple myeloma in a subject, comprising introducing the circular RNA encoding BCMA CAR and comprising an IRES and/or pharmaceutical composition thereof disclosed herein.

In some embodiments, provided herein is a method of treating and/or preventing a solid tumor in a subject, comprising introducing the circular RNA and/or pharmaceutical composition thereof disclosed herein. In some embodiments, provided herein is a method of treating a solid tumor in a subject, comprising introducing the circular RNA encoding HER2 CAR and/or pharmaceutical composition thereof disclosed herein. In some embodiments, provided herein is a method of treating a solid tumor in a subject, comprising introducing the circular RNA encoding HER2 CAR and comprising an IRES and/or pharmaceutical composition thereof disclosed herein.

In 2022, Mackensen et al. reported a compassionate-use anti-CD19 CAR T cell therapy for refractory systemic lupus erythematosus in which autologous T cells from SLE patients “were transduced with a lentiviral anti-CD19 CAR vector, expanded and reinfused . . . into the patients after lymphodepletion. CAR T cells expanded in vivo, led to deep depletion of B cells, improvement of clinical symptoms and normalization of laboratory parameters including seroconversion of anti-double-stranded DNA antibodies. Remission of SLE according to DORIS criteria was achieved in all five patients after 3 months and the median (range) Systemic Lupus Erythematosus Disease Activity Index score after 3 months was 0 (2).” Mackensen et al., Anti-CD19 CAR T cell therapy for refractory systemic lupus erythematosus, Nature Medicine (2022); see also Nunez et al., Cytokine and reactivity profiles in SLE patients following anti-CD19 CART therapy, Molecular Therapy (2023); the contents of both of which are hereby incorporated by reference in their entireties.

In some embodiments, provided herein is a method of treating and/or preventing an autoimmune disease, e.g., a B cell mediated autoimmune disease, e.g., lupus, in a subject, comprising introducing the circular RNA encoding CD19 CAR and/or pharmaceutical composition thereof disclosed herein. In some embodiments, provided herein is a method of treating lupus in a subject, comprising introducing the circular RNA encoding CD19 CAR and/or pharmaceutical composition thereof disclosed herein. In some embodiments, provided herein is a method of treating SLE in a subject, comprising introducing the circular RNA encoding CD19 CAR and/or pharmaceutical composition thereof disclosed herein.

In some embodiments, provided herein is a method of treating and/or preventing an autoimmune disease, e.g., a B cell mediated autoimmune disease, e.g., lupus, in a subject, comprising introducing the circular RNA encoding CD19 CAR and comprising an IRES and/or pharmaceutical composition thereof disclosed herein. In some embodiments, provided herein is a method of treating lupus in a subject, comprising introducing the circular RNA encoding CD19 CAR and comprising an IRES and/or pharmaceutical composition thereof disclosed herein. In some embodiments, provided herein is a method of treating SLE in a subject, comprising introducing the circular RNA encoding CD19 CAR and comprising an IRES and/or pharmaceutical composition thereof disclosed herein.

In some embodiments, provided herein is a method of treating and/or preventing an autoimmune disease, e.g., a B cell mediated autoimmune disease, e.g., lupus, in a subject, comprising introducing the circular RNA and/or pharmaceutical composition thereof disclosed herein. In some embodiments, provided herein is a method of treating lupus in a subject, comprising introducing the circular RNA and/or pharmaceutical composition thereof disclosed herein. In some embodiments, provided herein is a method of treating SLE in a subject, comprising introducing the circular RNA and/or pharmaceutical composition thereof disclosed herein.

In some embodiments, provided herein is a method of treating and/or preventing an autoimmune disease, e.g., a B cell mediated autoimmune disease, e.g., lupus, in a subject, comprising introducing the circular RNA encoding CD19 CAR and/or pharmaceutical composition thereof disclosed herein. In some embodiments, provided herein is a method of treating lupus in a subject, comprising introducing the circular RNA encoding CD19 CAR and/or pharmaceutical composition thereof disclosed herein. In some embodiments, provided herein is a method of treating SLE in a subject, comprising introducing the circular RNA encoding CD19 CAR and/or pharmaceutical composition thereof disclosed herein.

In some embodiments, provided herein is a method of treating and/or preventing an autoimmune disease, e.g., a B cell mediated autoimmune disease, e.g., lupus, in a subject, comprising introducing the circular RNA encoding CD19 CAR and comprising an IRES and/or pharmaceutical composition thereof disclosed herein. In some embodiments, provided herein is a method of treating lupus in a subject, comprising introducing the circular RNA encoding CD19 CAR and comprising an IRES and/or pharmaceutical composition thereof disclosed herein. In some embodiments, provided herein is a method of treating SLE in a subject, comprising introducing the circular RNA encoding CD19 CAR and comprising an IRES and/or pharmaceutical composition thereof disclosed herein.

The transfer vehicles may preferentially distribute to specific target cells such as immune cells (e.g., T cells, NK cells, macrophages, etc.), or in the heart, lungs, kidneys, liver, and spleen. In some embodiments, the compositions distribute into the cells of the liver or spleen to facilitate the delivery and the subsequent expression of the circular RNA comprised therein by the cells of the liver (e.g., hepatocytes) or the cells of spleen (e.g., immune cells). The targeted cells may function as a biological “reservoir” or “depot” capable of producing, and systemically excreting a functional protein or enzyme. Accordingly, in one embodiment of the disclosure, the transfer vehicle may target hepatocytes or immune cells and/or preferentially distribute to the cells of the liver or spleen upon delivery. In an embodiment, following transfection of the target hepatocytes or immune cells, the circular RNA loaded in the vehicle is translated and a functional protein product is produced, excreted and systemically distributed. In other embodiments, cells other than hepatocytes (e.g., lung, spleen, heart, ocular, or cells of the central nervous system) can serve as a depot location for protein production. The compositions may also be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

In some embodiments, provided herein is a method of treating and/or preventing a disease or disorder in a subject, comprising introducing the circular RNA and/or pharmaceutical composition thereof to a T cell in the subject. In some embodiments, provided herein is a method of treating a disease or disorder in a subject, comprising introducing the circular RNA and/or pharmaceutical composition thereof to a helper T cell in the subject. In some embodiments, provided herein is a method of treating a disease or disorder in a subject, comprising introducing the circular RNA and/or pharmaceutical composition thereof to a cytotoxic T cell in the subject.

In some embodiments, provided herein is a method of treating and/or preventing a disease or disorder in a subject, comprising introducing the circular RNA and/or pharmaceutical composition thereof to a NK cell in the subject.

In some embodiments, provided herein is a method of treating and/or preventing a disease or disorder in a subject, comprising introducing the circular RNA and/or pharmaceutical composition thereof to a macrophage in the subject.

In some embodiments, provided herein is a method of treating and/or preventing a disease or disorder in a subject, comprising introducing the circular RNA and/or pharmaceutical composition thereof to a myeloid cell in the subject.

In some embodiments, provided herein is a method of treating and/or preventing a disease or disorder in a subject, comprising introducing the circular RNA and/or pharmaceutical composition thereof to a monocyte in the subject.

In some embodiments, provided herein is a method of treating and/or preventing a disease or disorder in a subject, comprising introducing the circular RNA and/or pharmaceutical composition thereof to a CD3+ cell in the subject.

In some embodiments, provided herein is a method of treating and/or preventing a disease or disorder in a subject, comprising introducing the circular RNA and/or pharmaceutical composition thereof to a CD4+ cell in the subject.

In some embodiments, provided herein is a method of treating and/or preventing a disease or disorder in a subject, comprising introducing the circular RNA and/or pharmaceutical composition thereof to a CD8+ cell in the subject.

In some embodiments, provided herein is a method of treating and/or preventing a disease or disorder in a subject, comprising introducing the circular RNA and/or pharmaceutical composition thereof to a CD14+ cell in the subject.

In some embodiments, provided herein is a method of treating and/or preventing a disease or disorder in a subject, comprising introducing the circular RNA and/or pharmaceutical composition thereof to a CD16+ cell in the subject.

In some embodiments, provided herein is a method of treating and/or preventing a disease or disorder in a subject, comprising introducing the circular RNA and/or pharmaceutical composition thereof to a CD33+ cell in the subject.

In some embodiments, provided herein is a method of treating and/or preventing a disease or disorder in a subject, comprising introducing the circular RNA and/or pharmaceutical composition thereof to a CD33+CD14+ cell in the subject.

In some embodiments, provided herein is a method of treating and/or preventing a disease or disorder in a subject, comprising introducing the circular RNA and/or pharmaceutical composition thereof to a CD33+CD64+ cell in the subject.

In some embodiments, provided herein is a method of treating and/or preventing a disease or disorder in a subject, comprising introducing the circular RNA and/or pharmaceutical composition thereof to a CD56+ cell in the subject.

In some embodiments, provided herein is a method of treating and/or preventing a disease or disorder in a subject, comprising introducing the circular RNA and/or pharmaceutical composition thereof to a CD11B+ cell in the subject.

In certain embodiments the therapeutic agents provided herein (i.e., circular RNA constructs and compositions thereof) and the transfer vehicles (e.g., lipid nanoparticles) exhibit an enhanced (e.g., increased) ability to transfect one or more target cells. Accordingly, also provided herein are methods of transfecting one or more target cells. Such methods generally comprise the step of contacting the one or more target cells with the compounds and/or pharmaceutical compositions disclosed herein such that the one or more target cells are transfected with the circular RNA encapsulated therein. As used herein, the terms “transfect” or “transfection” refer to the intracellular introduction of one or more encapsulated materials (e.g., nucleic acids and/or polynucleotides) into a cell, or preferably into a target cell. The term “transfection efficiency” refers to the relative amount of such encapsulated material (e.g., polynucleotides) up-taken by, introduced into and/or expressed by the target cell which is subject to transfection. In some embodiments, transfection efficiency may be estimated by the amount of a reporter polynucleotide product produced by the target cells following transfection. In some embodiments, a transfer vehicle has high transfection efficiency. In some embodiments, a transfer vehicle has at least about 10%, 20%, 30%, 40%, 50%, 60%, 70%, 80%, or 90% transfection efficiency.

In certain embodiments, the circular RNA constructs or compositions comprising circular RNA constructs disclosed herein are administered to a subject. In some embodiments, administration occurs twice a day, daily or every other day. In certain embodiments, administration occurs twice a week, once a week, every ten days, every two weeks, every three weeks, every four weeks, once a month, every six weeks, every eight weeks, every three months, every four months, every six months, every eight months, every nine months or annually. In some embodiments, administration occurs every other day. In some embodiments, administration occurs once a week (i.e., weekly). In some embodiments, administration occurs every two weeks (i.e., every other week or biweekly, q2w). In some embodiments, administration occurs less frequently and/or at a lower dose than as required for a suitable comparator, for example, a corresponding linear RNA.

In certain embodiments, the therapeutic agents provided herein (i.e., circular RNA constructs and compositions thereof) are co-administered with one or more additional therapeutic agents e.g., in the same pharmaceutical composition or in separate pharmaceutical compositions. In some embodiments, the therapeutic agent provided herein can be administered first and the one or more additional therapeutic agents can be administered second, or vice versa. Alternatively, the therapeutic agent provided herein and the one or more additional therapeutic agents can be administered simultaneously. In some embodiments, the therapeutic agents are co-administered sufficiently close in time such that the therapeutic agent provided herein can enhance the effect of the one or more additional therapeutic agents, or vice versa.

In certain embodiments, the methods further comprise lymphodepleting the subject prior to administering the therapeutic agent. Examples of lymphodepletion include, but may not be limited to, nonmyeloablative lymphodepleting chemotherapy, myeloablative lymphodepleting chemotherapy, total body irradiation, etc.

In some embodiments, the subject is a mammal. In some embodiments, the mammal referred to herein can be any mammal, including, but not limited to, mammals of the order Rodentia, such as mice and hamsters, or mammals of the order Logomorpha, such as rabbits. The mammals may be from the order Carnivora, including Felines (cats) and Canines (dogs). The mammals may be from the order Artiodactyla, including Bovines (cows) and Swines (pigs), or of the order Perssodactyla, including Equines (horses). The mammals may be of the order Primates, Ceboids, or Simoids (monkeys) or of the order Anthropoids (humans and apes). Preferably, the mammal is a human.

### Examples

The following examples are provided to illustrate certain disclosed embodiments and are not to be construed as limiting the scope of this disclosure in any way.

### Example 1: Production of Lipid Nanoparticle Compositions

In order to investigate safe and efficacious lipid nanoparticle compositions for use in the delivery of circular RNA to cells, a range of formulations were prepared and tested. Specifically, the particular elements and ratios thereof in the lipid component of nanoparticle compositions were optimized.

Nanoparticles can be made in a one fluid stream or with mixing processes such as microfluidics and T-junction mixing of two fluid streams, one of which contains the circular RNA and the other has the lipid components.

Lipid compositions can be prepared, including by combining an ionizable lipid, optionally a helper lipid (such as DOPE, DSPC, or oleic acid obtainable from Avanti Polar Lipids, Alabaster, AL), a PEG lipid (such as 1,2-dimyristoyl-sn-glycerol methoxypolyethylene glycol, also known as PEG-DMG, obtainable from Avanti Polar Lipids, Alabaster, AL), and a structural lipid such as cholesterol at concentrations of about, e.g., 40 or 50 mM in a solvent, e.g., ethanol. Solutions should be refrigerated for storage at, for example, −20° C. Lipids are combined to yield desired molar ratios (see, for example, Tables 4a and 4b below) and diluted with water and ethanol to a final lipid concentration of e.g., between about 5.5 mM and about 25 mM.

TABLE 4a
Lipid Nanoparticle Formulations

| Formulation | |
|---|---|
| number | Description |
| 1 | Aliquots of 50 mg/mL ethanolic solutions of C12-200, DOPE, Chol and DMG- |
| | PEG2K (40:30:25:5) are mixed and diluted with ethanol to 3 mL final volume. |
| | Separately, an aqueous buffered solution (10 mM citrate/150 mM NaCl, pH 4.5) of |
| | circRNA is prepared from a 1 mg/mL stock. The lipid solution is injected rapidly |
| | into the aqueous circRNA solution and shaken to yield a final suspension in 20% |
| | ethanol. The resulting nanoparticle suspension is filtered, diafiltrated with 1 × PBS |
| | (pH 7.4), concentrated and stored at 2-8° C |
| 2 | Aliquots of 50 mg/mL ethanolic solutions of DODAP, DOPE, cholesterol and |
| | DMG-PEG2K (18:56:20:6) are mixed and diluted with ethanol to 3 mL final |
| | volume. Separately, an aqueous buffered solution (10 mM citrate/150 mM NaCl, |
| | pH 4.5) of EPO circRNA is prepared from a 1 mg/mL stock. The lipid solution is |
| | injected rapidly into the aqueous circRNA solution and shaken to yield a final |
| | suspension in 20% ethanol. The resulting nanoparticle suspension is filtered, |
| | diafiltrated with 1 × PBS (pH 7.4), concentrated and stored at 2-8° C. Final |
| | concentration = 1.35 mg/mL EPO circRNA (encapsulated). Zave = 75.9 nm |
| | (Dv(50) = 57.3 nm; Dv(90) = 92.1 nm). |
| 3 | Aliquots of 50 mg/mL ethanolic solutions of HGT4003, DOPE, cholesterol and |
| | DMG-PEG2K (50:25:20:5) are mixed and diluted with ethanol to 3 mL final |
| | volume. Separately, an aqueous buffered solution (10 mM citrate/150 mM NaCl, |
| | pH 4.5) of circRNA is prepared from a 1 mg/mL stock. The lipid solution is injected |
| | rapidly into the aqueous circRNA solution and shaken to yield a final suspension in |
| | 20% ethanol. The resulting nanoparticle suspension is filtered, diafiltrated with |
| | 1 × PBS (pH 7.4), concentrated and stored at 2-8° C. |
| 4 | Aliquots of 50 mg/mL ethanolic solutions of ICE, DOPE and DMG-PEG2K |
| | (70:25:5) are mixed and diluted with ethanol to 3 mL final volume. Separately, an |
| | aqueous buffered solution (10 mM citrate/150 mM NaCl, pH 4.5) of circRNA is |
| | prepared from a 1 mg/mL stock. The lipid solution is injected rapidly into the |
| | aqueous circRNA solution and shaken to yield a final suspension in 20% ethanol. |
| | The resulting nanoparticle suspension is filtered, diafiltrated with 1 × PBS (pH 7.4), |
| | concentrated and stored at 2-8° C. |
| 5 | Aliquots of 50 mg/mL ethanolic solutions of HGT5000, DOPE, cholesterol and |
| | DMG-PEG2K (40:20:35:5) are mixed and diluted with ethanol to 3 mL final |
| | volume. Separately, an aqueous buffered solution (10 mM citrate/150 mM NaCl, |
| | pH 4.5) of EPO circRNA is prepared from a 1 mg/mL stock. The lipid solution is |
| | injected rapidly into the aqueous circRNA solution and shaken to yield a final |
| | suspension in 20% ethanol. The resulting nanoparticle suspension is filtered, |
| | diafiltrated with 1 × PBS (pH 7.4), concentrated and stored at 2-8° C. Final |
| | concentration = 1.82 mg/mL EPO mRNA (encapsulated). Zave = 105.6 nm |
| | (Dv(50) = 53.7 nm; Dv(90) = 157 nm). |
| 6 | Aliquots of 50 mg/mL ethanolic solutions of HGT5001, DOPE, cholesterol and |
| | DMG-PEG2K (40:20:35:5) are mixed and diluted with ethanol to 3 mL final |
| | volume. Separately, an aqueous buffered solution (10 mM citrate/150 mM NaCl, |
| | pH 4.5) of EPO circRNA is prepared from a 1 mg/mL stock. The lipid solution is |
| | injected rapidly into the aqueous circRNA solution and shaken to yield a final |
| | suspension in 20% ethanol. The resulting nanoparticle suspension is filtered, |
| | diafiltrated with 1 × PBS (pH 7.4), concentrated and stored at 2-8° C. |
| 7 | Aliquots of 50 mg/mL ethanolic solutions of HGT5001, DOPE, cholesterol and |
| | DMG-PEG2K (35:16:46.5:2.5) are mixed and diluted with ethanol to 3 mL final |
| | volume. Separately, an aqueous buffered solution (10 mM citrate/150 mM NaCl, |
| | pH 4.5) of EPO circRNA is prepared from a 1 mg/mL stock. The lipid solution is |
| | injected rapidly into the aqueous circRNA solution and shaken to yield a final |
| | suspension in 20% ethanol. The resulting nanoparticle suspension is filtered, |
| | diafiltrated with 1 × PBS (pH 7.4), concentrated and stored at 2-8° C. |
| 8 | Aliquots of 50 mg/mL ethanolic solutions of HGT5001, DOPE, cholesterol and |
| | DMG-PEG2K (40:10:40:10) are mixed and diluted with ethanol to 3 mL final |
| | volume. Separately, an aqueous buffered solution (10 mM citrate/150 mM NaCl, |
| | pH 4.5) of EPO circRNA is prepared from a 1 mg/mL stock. The lipid solution is |
| | injected rapidly into the aqueous circRNA solution and shaken to yield a final |
| | suspension in 20% ethanol. The resulting nanoparticle suspension is filtered, |
| | diafiltrated with 1 × PBS (pH 7.4), concentrated and stored at 2-8° C. |
| 9 | Aliquots of 50 mg/mL ethanolic solutions of LP1, DSPC, cholesterol and DMG- |
| | PEG2K (45:9:44:2) are mixed and diluted with ethanol to 3 mL final volume. |
| | Separately, an aqueous buffered solution (50 mM Na Acetate, pH 4.5) of FLuc, |
| | hEPO, micro-dystrophin, mini-dystrophin, or dystrophin circRNA is prepared from |
| | a 1 mg/mL stock. The lipid solution is injected rapidly into the aqueous circRNA |
| | solution and shaken to yield a final suspension in 25% ethanol. The resulting |
| | nanoparticle suspension is filtered, diafiltrated with 1 × PBS (pH 7.4), concentrated |
| | and stored at 2-8° C. |
| 10 | Aliquots of 50 mg/mL ethanolic solutions of LP1, DSPC, cholesterol and DMG- |
| | PEG2K, and DMSO solution of Ethyl Lauroyl Arginate (38.25:7.65:37.4:1.7:15) |
| | are mixed and diluted with ethanol to 3 mL final volume. Separately, an aqueous |
| | buffered solution (50 mM Bis-Tris, pH 7.0) of FLuc, hEPO, micro-dystrophin, |
| | mini-dystrophin, or dystrophin circRNA is prepared from a 1 mg/mL stock. The |
| | lipid solution is injected rapidly into the aqueous circRNA solution and shaken to |
| | yield a final suspension in 25% ethanol. The resulting nanoparticle suspension is |
| | filtered, diafiltrated with 1 × PBS (pH 7.4), concentrated and stored at 2-8° C. |
| 11 | Aliquots of 50 mg/mL ethanolic solutions of SM-102, DSPC, cholesterol and |
| | DMG-PEG2K (50:10:38.5:1.5) are mixed and diluted with ethanol to 3 mL final |
| | volume. Separately, an aqueous buffered solution (6.25 mM Na Acetate, pH 4.5) of |
| | FLuc, hEPO, micro-dystrophin, mini-dystrophin, or dystrophin circRNA is |
| | prepared from a 1 mg/mL stock. The lipid solution is injected rapidly into the |
| | aqueous circRNA solution and shaken to yield a final suspension in 25% ethanol. |
| | The resulting nanoparticle suspension is filtered, diafiltrated with 1 × PBS (pH 7.4), |
| | concentrated and stored at 2-8° C. |
| 12 | Aliquots of 50 mg/mL ethanolic solutions of SM-102, DSPC, cholesterol and |
| | DMG-PEG2K, and DMSO solution of Ethyl Lauroyl Arginate |
| | (42.5:8.5:32,73:1.275:15) are mixed and diluted with ethanol to 3 mL final volume. |
| | Separately, an aqueous buffered solution (20 mM Na Acetate, pH 4.5) of FLuc, |
| | hEPO, micro-dystrophin, mini-dystrophin, or dystrophin circRNA is prepared from |
| | a 1 mg/mL stock. The lipid solution is injected rapidly into the aqueous circRNA |
| | solution and shaken to yield a final suspension in 25% ethanol. The resulting |
| | nanoparticle suspension is filtered, diafiltrated with 1 × PBS (pH 7.4), concentrated |
| | and stored at 2-8° C. |

In some embodiments, the transfer vehicle has a formulation as described in Table 4b.

TABLE 4b
Exemplary Lipid Vehicle Formulations

| Composition | |
|---|---|
| (mol %) | Components |
| 40:20:38.5:1.5 | Compound:Phospholipid:Phytosterol*:PEG-DMG |
| 45:15:38.5:1.5 | Compound:Phospholipid:Phytosterol*:PEG-DMG |
| 50:10:38.5:1.5 | Compound:Phospholipid:Phytosterol*:PEG-DMG |
| 55:5:38.5:1.5 | Compound:Phospholipid:Phytosterol*:PEG-DMG |
| 60:5:33.5:1.5 | Compound:Phospholipid:Phytosterol*:PEG-DMG |
| 45:20:33.5:1.5 | Compound:Phospholipid:Phytosterol*:PEG-DMG |
| 50:20:28.5:1.5 | Compound:Phospholipid:Phytosterol*:PEG-DMG |
| 55:20:23.5:1.5 | Compound:Phospholipid:Phytosterol*:PEG-DMG |
| 60:20:18.5:1.5 | Compound:Phospholipid:Phytosterol*:PEG-DMG |
| 40:15:43.5:1.5 | Compound:Phospholipid:Phytosterol*:PEG-DMG |
| 50:15:33.5:1.5 | Compound:Phospholipid:Phytosterol*:PEG-DMG |
| 55:15:28.5:1.5 | Compound:Phospholipid:Phytosterol*:PEG-DMG |
| 60:15:23.5:1.5 | Compound:Phospholipid:Phytosterol*:PEG-DMG |
| 40:10:48.5:1.5 | Compound:Phospholipid:Phytosterol*:PEG-DMG |
| 45:10:43.5:1.5 | Compound:Phospholipid:Phytosterol*:PEG-DMG |
| 55:10:33.5:1.5 | Compound:Phospholipid:Phytosterol*:PEG-DMG |
| 60:10:28.5:1.5 | Compound:Phospholipid:Phytosterol*:PEG-DMG |
| 40:5:53.5:1.5 | Compound:Phospholipid:Phytosterol*:PEG-DMG |
| 45:5:48.5:1.5 | Compound:Phospholipid:Phytosterol*:PEG-DMG |
| 50:5:43.5:1.5 | Compound:Phospholipid:Phytosterol*:PEG-DMG |
| 40:20:40:0 | Compound:Phospholipid:Phytosterol*:PEG-DMG |
| 45:20:35:0 | Compound:Phospholipid:Phytosterol*:PEG-DMG |
| 50:20:30:0 | Compound:Phospholipid:Phytosterol*:PEG-DMG |
| 55:20:25:0 | Compound:Phospholipid:Phytosterol*:PEG-DMG |
| 60:20:20:0 | Compound:Phospholipid:Phytosterol*:PEG-DMG |
| 40:15:45:0 | Compound:Phospholipid:Phytosterol*:PEG-DMG |

For nanoparticle compositions including circRNA, solutions of the circRNA at concentrations of 0.1 mg/ml in deionized water are diluted in a buffer, e.g., 50 mM sodium citrate buffer at a pH between 3 and 4 to form a stock solution. Alternatively, solutions of the circRNA at concentrations of 0.15 mg/ml in deionized water are diluted in a buffer, e.g., 6.25 mM sodium acetate buffer at a pH between 3 and 4.5 to form a stock solution.

Nanoparticle compositions including a circular RNA and a lipid component are prepared by combining the lipid solution with a solution including the circular RNA at lipid component to circRNA wt:wt ratios between about 5:1 and about 50:1. The lipid solution is rapidly injected using, e.g., a NanoAssemblr microfluidic based system at flow rates between about 10 ml/min and about 18 ml/min or between about 5 ml/min and about 18 ml/min into the circRNA solution, to produce a suspension with a water to ethanol ratio between about 1:1 and about 4:1.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, IL) with a molecular weight cutoff of 10 kDa or 20 kDa. The formulations are then dialyzed overnight at 4° C. The resulting nanoparticle suspension is filtered through 0.2 μm sterile filters (Sarstedt, Nümbrecht, Germany) into glass vials and sealed with crimp closures. Nanoparticle composition solutions of 0.01 mg/ml to 0.15 mg/ml are generally obtained.

The method described above induces nano-precipitation and particle formation.

Alternative processes including, but not limited to, T-junction and direct injection, may be used to achieve the same nano-precipitation.

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of circRNA in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, CA). The concentration of circRNA in the nanoparticle composition can be calculated based on the extinction coefficient of the circRNA used in the composition and on the difference between the absorbance at a wavelength of, for example, 260 nm and the baseline value at a wavelength of, for example, 330 nm.

A QUANT-IT™ RIBOGREEN® RNA assay (Invitrogen Corporation Carlsbad, CA) can be used to evaluate the encapsulation of circRNA by the nanoparticle composition. The samples are diluted to a concentration of approximately 5 μg/mL or 1 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2-4% Triton X-100 solution is added to the wells. The plate is incubated at a temperature of 37° C. for 15 minutes. The RIBOGREEN® reagent is diluted 1:100 or 1:200 in TE buffer, and 100 μL of this solution is added to each well. The fluorescence intensity can be measured using a fluorescence plate reader (Wallac Victor 1420 Multilabel Counter; Perkin Elmer, Waltham, MA) at an excitation wavelength of, for example, about 480 nm and an emission wavelength of, for example, about 520 nm. The fluorescence values of the reagent blank are subtracted from that of each of the samples and the percentage of free circRNA is determined by dividing the fluorescence intensity of the intact sample (without addition of Triton X-100) by the fluorescence value of the disrupted sample (caused by the addition of Triton X-100).

### Example 2: Synthesis of Ionizable Lipids

### Example 2.1 Synthesis of heptadecan-9-yl 8-((3-hydroxypropyl) (2-hydroxytetradecyl)amino)octanoate (Table 3, Lipid 1)

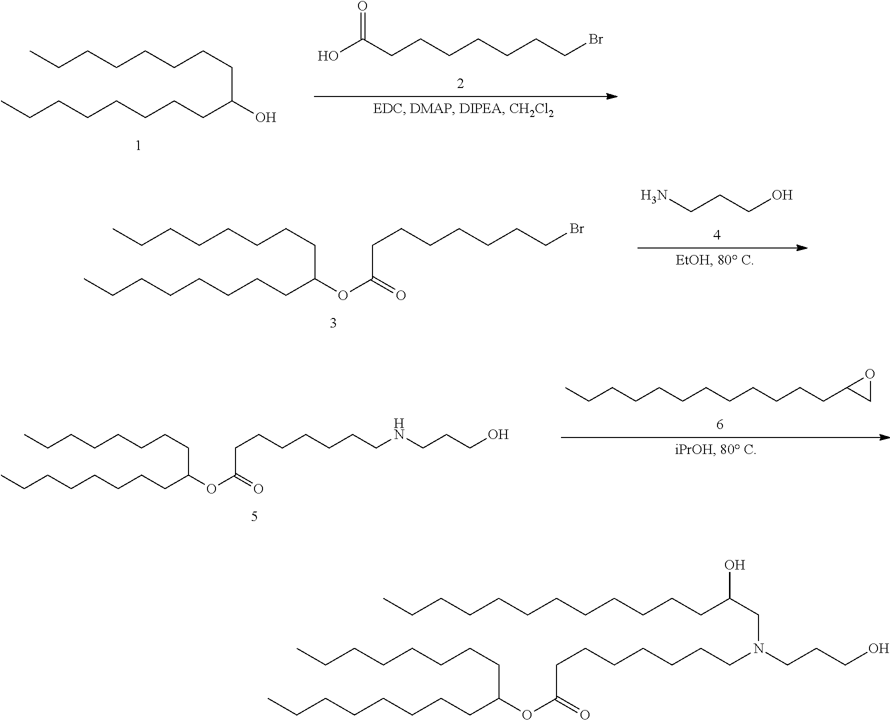

### Example 2.1.1 Synthesis of heptadecan-9-yl 8-bromooctanoate (3)

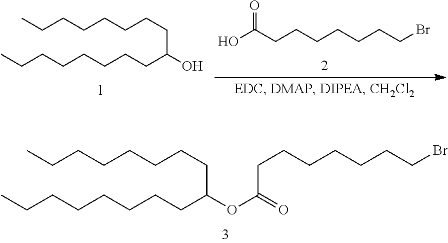 [SMILES: CCCCCCCCC(O)CCCCCCCC ; O=C(O)CCCCCCCBr ; CCCCCCCCC(CCCCCCCC)OC(=O)CCCCCCCBr]

To a mixture of 8-bromooctanoic acid 2 (10 g, 44.82 mmol) and heptadecan-9-ol 1 (9.6 g, 37.35 mmol) in CH₂C12 (300 mL) was added DMAP (900 mg, 7.48 mmol), DIPEA (26 mL, 149.7 mmol) and EDC (10.7 g, 56.03 mmol). The reaction was stirred at room temperature overnight. After concentration of the reaction mixture, the crude residue was dissolved in ethyl acetate (300 mL), washed with 1N HCl, sat. NaHCO₃, water and Brine. The organic layer was dried over anhydrous Na₂SO4. The solvent was evaporated, and the crude residue was purified by flash chromatography (SiO₂: Hexane=100% to 30% of EtOAc in Hexane) and colorless oil product 3 was obtained (5 g, 29%).

1H NMR (300 MHz, CDCl₃): δ ppm 4.86 (m, 1H), 3.39 (t, J=7.0 Hz, 2H), 2.27 (t, J=7.6 Hz, 2H), 1.84 (m, 2H), 1.62 (m, 2H), 1.5-1.4 (m, 8H), 1.35-1.2 (m, 26H), 0.87 (t, J=6.7 Hz, 6H).

### Example 2.1.2 Synthesis of heptadecan-9-yl 8-((3-hydroxypropyl)amino)octanoate (5)

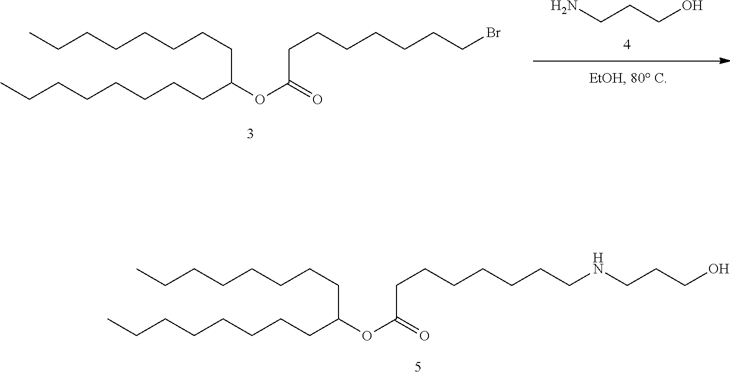 [SMILES: NCCCO ; CCCCCCCCC(CCCCCCCC)OC(=O)CCCCCCCBr ; CCCCCCCCC(CCCCCCCC)OC(=O)CCCCCCCNCCCO]

A solution of 1-octylnonyl 8-bromooctanoate 3 (7.4 g, 16.03 mmol) in EtOH (200 mL) was added 3-amino-1-propanol 4 (24.4 mL, 320 mmol) and the reaction solution was heated at 70° C. overnight. MS showed the expected product: [APCI]: [MH]+456.4. After concentration of the reaction mixture, the crude residue was dissolved in methyl tert-butyl ether (500 mL), washed with sat. NaHCO3, water and Brine. The organic layer was dried over anhydrous Na2SO4. The solvent was evaporated, and the crude residue was purified by flash chromatography (SiO₂: CH₂Cl2=100% to 10% of MeOH in CH₂Cl2 with 1% NH4OH) and colorless oil product 5 was obtained (6.6 g, 88%).

1H NMR (300 MHZ, CDCl3): δ ppm 4.84 (m, 1H), 3.80 (t, J=5.5 Hz, 2H), 2.87 (t, J=5.76 Hz, 2H), 2.59 (t, J=7.2 Hz, 2H), 2.27 (t, J=7.6 Hz, 2H), 1.68 (m, 2H), 1.62 (m, 2H), 1.5-1.4 (m, 5H), 1.35-1.2 (m, 32H), 0.87 (t, J=6.7 Hz, 6H). MS (APCI+): 456.4 (M+1).

### Example 2.1.3 Synthesis of heptadecan-9-yl 8-((3-hydroxypropyl) (2-hydroxytetradecyl)amino)octanoate (7)

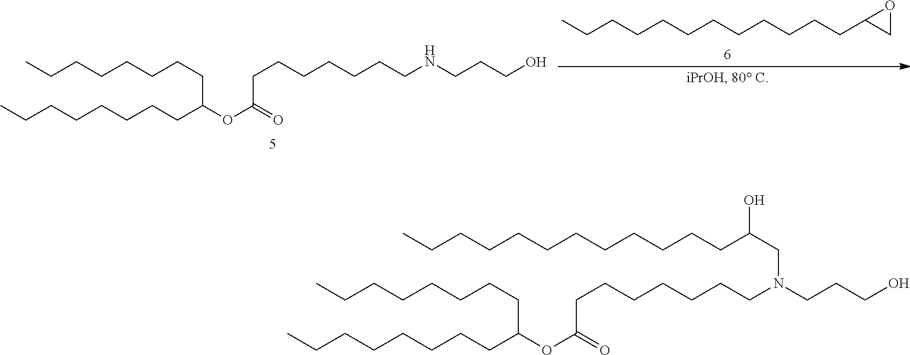 [SMILES: CCCCCCCCCCCCC1CO1 ; CCCCCCCCC(CCCCCCCC)OC(=O)CCCCCCCNCCCO ; CCCCCCCCCCCCC(O)CN(CCCO)CCCCCCCC(=O)OC(CCCCCCCC)CCCCCCCC]

A mixture of compound 5 (6.6 g, 14.5 mmol) and 1,2-epoxytetradecane (3.68 g, 17.4 mmol) in isopropanol (150 mL) was heated to reflux for overnight. MS showed the expected product: [APCI]: [MH]+668.6. The reaction mixture was concentrated, and crude product was purified flash chromatography (SiO₂: CH₂Cl2=100% to 10% of MeOH in CH2Cl2 with 1% NH4OH) to obtained Lipid 10e-1 as colorless oil (6.34 g, 65%).

1H NMR (300 MHz, CDCl₃): δ ppm 4.85 (m, 1H), 3.76 (t, J=5.49 Hz, 2H), 3.68 (m, 1H), 2.75 (m, 1H), 2.59 (m, 2H), 2.38 (m, 3H), 2.27 (m, 2H), 1.58-1.68 (m, 2H), 1.48 (m, 6H), 1.24 (m, 56H), 0.87 (m, 9H). MS (APCI+): 668.6 (M+1).

### Example 2.2 Synthesis of Di (undecan-3-yl) 8,8′-((3-hydroxypropyl)azanediyl)bis(7-hydroxyoctanoate) (Table 3, Lipid 7)

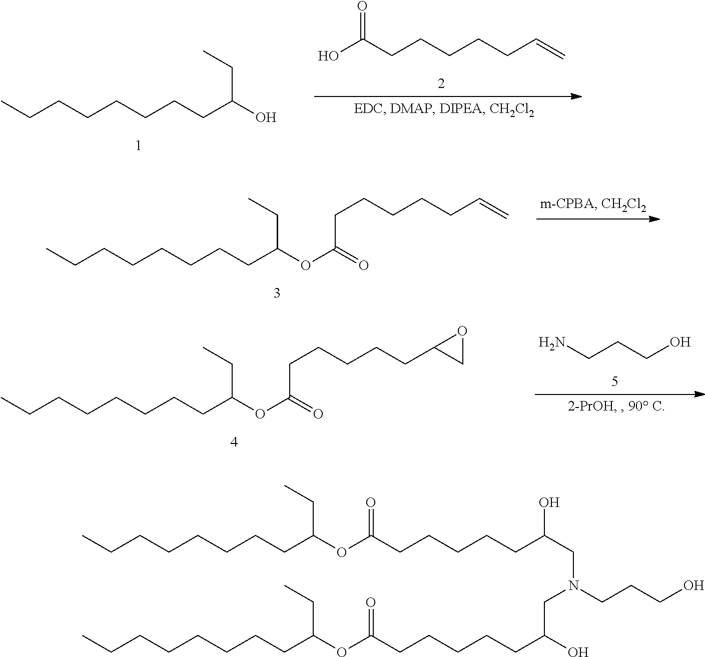

### Example 2.2.1 Synthesis of undecan-3-yl oct-7-enoate (3)

To a mixture of oct-7-enoic acid 2 (10 g, 70.3 mmol) and undecan-3-ol 1 (10 g, 58.6 mmol) in CH2Cl2 (300 mL) was added DMAP (1.4 g, 11.6 mmol), DIPEA (40 mL, 232 mmol) and EDC (16.9 g, 87.9 mmol). The reaction was stirred at room temperature overnight. After concentration of the reaction mixture, the crude residue was dissolved in tert-butylmethyl ether (500 mL), washed with IN HCl, sat. NaHCO₃, water and Brine. The organic layer was dried over anhydrous Na₂SO4. The solvent was evaporated and the crude residue was purified by flash chromatography (SiO₂: Hexane=100% to 20% of EtOAc in Hexane) and colorless oil product 3 was obtained (17.2 g, 98%).

1H NMR (300 MHz, CDCl₃): δ ppm 5.88-5.72 (m, 1H), 5.02-4.91 (m, 1H), 4.80 (m, 1H), 2.28 (t, J=7.4 Hz, 2H), 2.05-2.03 (m, 2H), 1.62-1.49 (m, 6H), 1.37-1.25 (m, 16H), 0.87 (t, J=7.4 Hz, 6H).

### Example 2.2.2 Synthesis of undecan-3-yl 6-(oxiran-2-yl) hexanoate (4)

To a mixture of undecan-3-yl oct-7-enoate 3 (17.2 g, 58.1 mmol) in CH₂C12 (300 mL) was added meta-chloroperoxybenzoic acid (mCPBA, <77%) (19.5 g, 87 mmol) in one portion at 0° C. ice-water bath. The reaction was stirred at room temperature overnight. The white precipitate (meta-benzoic acid) was filtered and the filtrate was diluted with CH₂C12 (200 mL), washed with 10% Na2S203, sat. NaHCO3, water and Brine. The organic layer was dried over anhydrous Na2SO4. The solvent was evaporated and the crude residue was purified by flash chromatography (SiO₂: Hexane=100% to 30% of EtOAc in Hexane) and colorless oil product 3 was obtained (17.1 g, 97%).

1H NMR (300 MHz, CDCl₃): δ ppm 4.80 (m, 1H), 2.89-2.86 (m, 1H), 3.39 (t, J=7.0 Hz, 2H), 2.74 (t, J=4.7 Hz, 1H), 2.47 (dd, J=4.9, 2.2 Hz, 1H), 2.28 (t, J=7.4 Hz, 1H), 1.74-1.46 (m, 10H), 1.35-1.2 (m, 13H) 0.87 (m, 6H).

### Example 2.2.3 Synthesis of Di(undecan-3-yl) 8,8′-((3-hydroxypropyl)azanediyl)bis(7-hydroxyoctanoate) (Table 3, lipid 7)

A solution of undecan-3-yl 6-(oxiran-2-yl) hexanoate 4 (8 g, 25.6 mmol) in isopropanol (50 mL) was added 3-amino-1-propanol (769.1 mg, 10.2 mmol) and the reaction solution was heated at 90° C. overnight. MS showed the expected product: [APCI]: [MH]+700.6. After concentration of the reaction mixture, the crude residue was purified by flash chromatography (SiO₂: CH₂Cl2=100% to 10% of MeOH in CH₂C12) and colorless oil product was obtained (5.1 g, 71%).

1H NMR (300 MHz, CDCl₃): δ ppm 4.81 (m, 2H), 3.80 (m, 2H), 3.73 (m, 2H), 2.78 (m, 2H), 2.52-2.43 (m, 4H), 2.28 (t, J=7.3 Hz, 2H), 1.68-1.48 (m, 15H), 1.35-1.17 (m, 37H), 0.88-0.83 (m, 12H). MS (APCI+): 700.6 (M+1).

### Example 2.3 Synthesis of ((3-(2-methyl-1H-imidazol-1-yl) propyl)azanediyl)bis(hexane-6,1-diyl)bis(2-hexyldecanoate) (Table 3, lipid 129) and ((3-(1H-imidazol-1-yl) propyl)azanediyl)bis(hexane-6,1-diyl)bis(2-hexyldecanoate) (Table 3, lipid 128)

In a 100 mL round bottom flask connected with condenser, 3-(1H-imidazol-1-yl) propan-1-amine (100 mg, 0.799 mmol) or 3-(2-methyl-1H-imidazol-1-yl) propan-1-amine (0.799 mmol), 6-bromohexyl 2-hexyldecanoate (737.2 mg, 1.757 mmol), potassium carbonate (485 mg, 3.515 mmol) and potassium iodide (13 mg, 0.08 mmol) were mixed in acetonitrile (30 mL), and the reaction mixture was heated to 80° C. for 48 h. The mixture was cooled to room temperature and was filtered through a pad of Celite. The filtrate was diluted with ethyl acetate. After washing with water, brine and dried over anhydrous sodium sulfate. The solvent was evaporated and the crude residue was purified by flash chromatography (SiO₂: CH₂Cl2=100% to 10% of methanol in CH₂C12) and colorless oil product was obtained (92 mg, 15%). Molecular formula of ((3-(1H-imidazol-1-yl) propyl)azanediyl)bis(hexane-6,1-diyl)bis(2-hexyldecanoate)) is C₅₀H₉₅N₃O₄ and molecular weight (Mw) is 801.7.

Reaction scheme for synthesis of ((3-(1H-imidazol-1-yl) propyl)azanediyl)bis(hexane-6,1-diyl)bis(2-hexyldecanoate)) (Table 3, lipid 128).

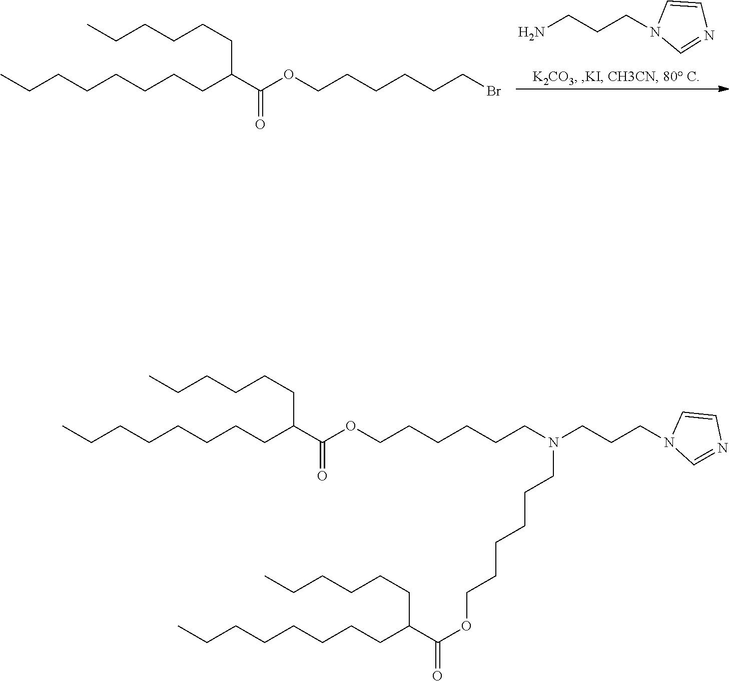 [SMILES: CCCCCCCCC(CCCCCC)C(=O)OCCCCCCBr ; NCCCn1ccnc1 ; CCCCCCCCC(CCCCCC)C(=O)OCCCCCCN(CCCCCCOC(=O)C(CCCCCC)CCCCCCCC)CCCn1ccnc1]

### Example 3: IRES and CodOP Screening

### A. Initial Screening

An initial screen assessing roughly 3,000 potential IRES sequence candidates was conducted. IRES sequence candidates were computationally identified from viral untranslated regions in partial and complete viral sequences available in the art (e.g., Genbank). Each DNA template was cloned to include an IRES sequence or a fragment. Engineered circular RNA (oRNA) was generated from the DNA template by in vitro transcription (IVT); and ORNA was subsequently purified from non-circular RNA components of the IVT reaction. Purified oRNA was nanoprecipitated with lipids to form LNP-ORNA constructs. Primary human skeletal myotubes (MYOs), primary human hepatocytes (PHHs), and activated primary human T cells (TCLs) were treated with formulated LNP-ORNA. Exemplary constructs included a 5′ enhanced intron element, a 5′ enhanced exon element, a punitive IRES, a coding element encoding Gaussia Luciferase, a 3′ enhanced exon element, and a 3′ enhanced intron element. See WO2022/261490. Protein expression in supernatant was assessed by luminescence measurements 48 hours following transfection. Total luminescent signal was normalized across replicate plates to control for plate-wide differences between replicates.

The top IRES sequences were selected based on protein expression and stability measurements.

Codons directed to CD19 were assessed via % Nalm6 Lysis and mean fluorescence intensity (MFI).

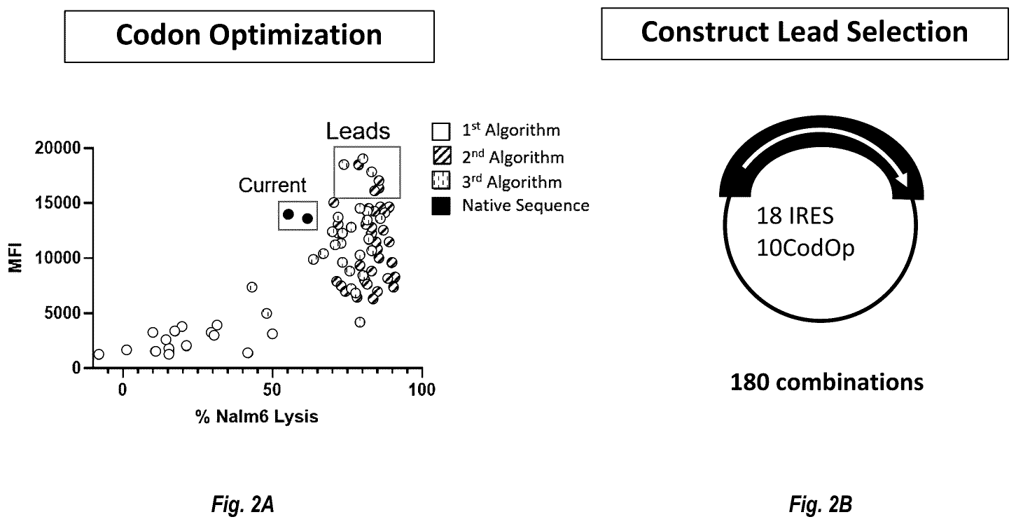

The results of the initial screen led to the selection of about 180 combinations of 18 IRESes (including the IRESes represented by SEQ ID NOs: 1-15 in Table IA) and 10 codons. After synthesis, 69 constructs based on combinations of 15 IRESes (SEQ ID NOs: 1-15 in Table 1A) and 5 codon-optimized sequences (SEQ ID NOs: 19-23 in Table 2A) were further tested. See FIGS. 2A-C and Table 5, below.

### B. Codon Optimization Assay

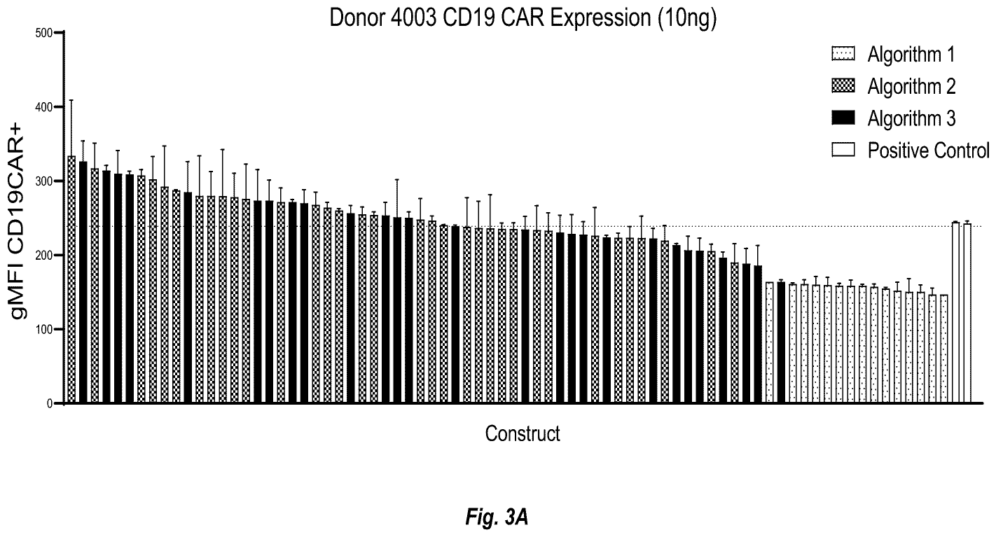

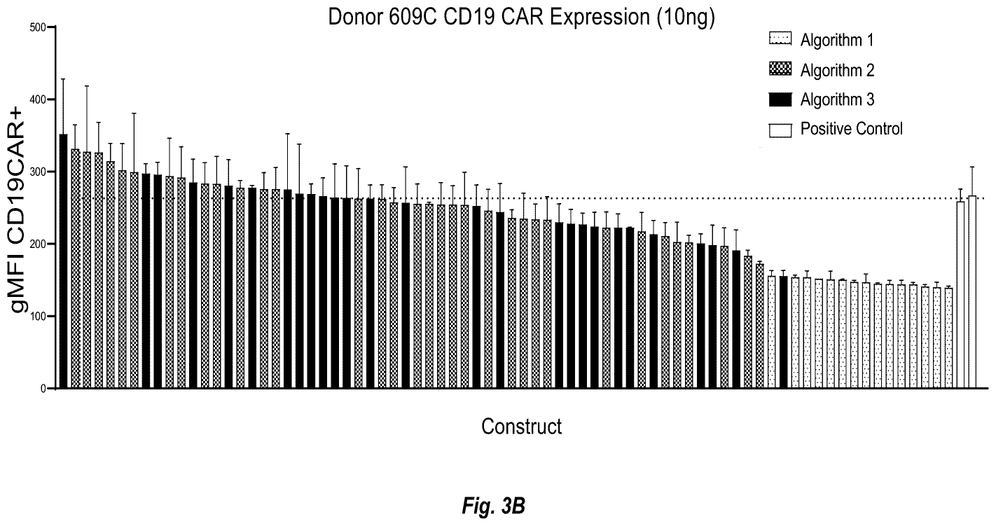

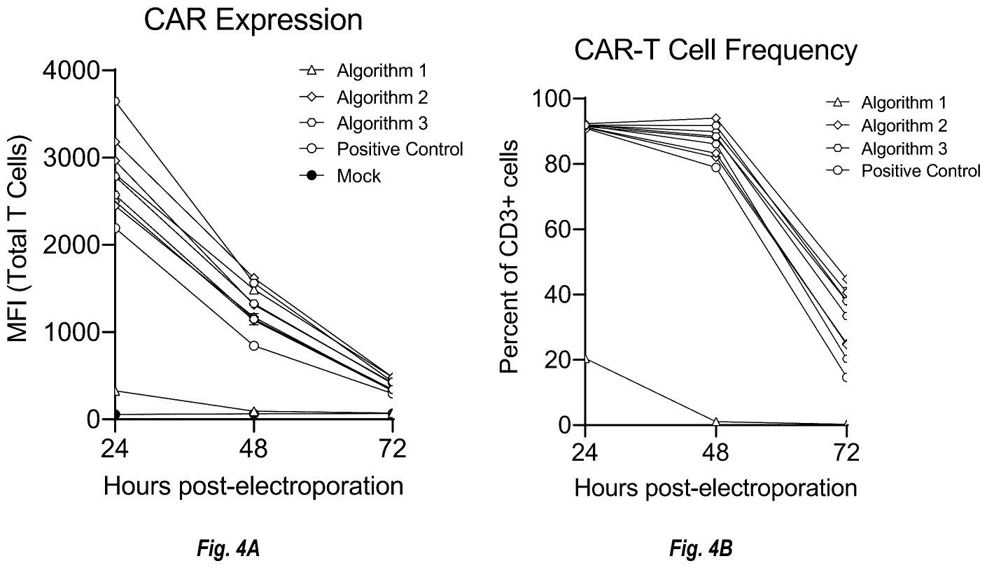

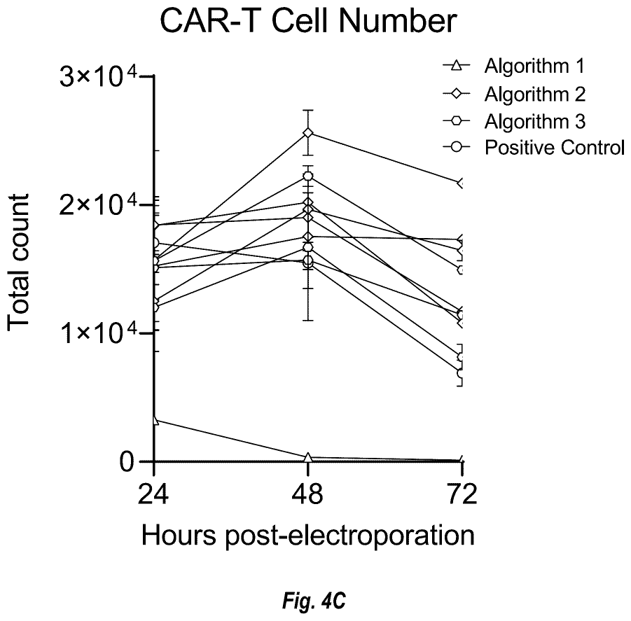

Codon optimization was performed using a ribosomal dwell time algorithm (target GC % of 48-54%), modified stability algorithm (target GC % of 57-62%) and a third algorithm based on reversed engineering of a known sequence (target GC % of 57-62%). As set forth in detail elsewhere herein, the algorithms optimized the particular codons based on GC %, unwanted sequence removal and low GC leader. CD3+ T cells from two healthy donors (4003; 609C) were activated with aCD3/CD28 tetrameric complexes for 3 days and then electroporated with CD19 CAR ORNA sequences. oRNA sequences were codon optimized using 3 algorithms (#1:15 sequences, #2:34 sequences, #3:27 sequences). CD19 CAR expression was analyzed via flow cytometry. CD19 CAR expression (gMFI) is plotted in rank-order for all sequences and color-coded by codon optimization algorithm. White bars indicate non-codon optimized CD19 CAR sequence. See FIGS. 3A and 3B and FIGS. 4A-4C.

The codon sequences were assessed and the top codon variants were elected for further evaluation. See Table 2A. The top codon variants were selected based on consistently robust expression stability and cytotoxicity against Nalm6 from previous optimization assays. From the 10 codons, 4 codons and the base were selected for further assessment: numbers 69, 24, 39, 45, and base (3276), which correspond to SEQ ID NOs: 19-23, as set forth in Table 2 based on assessing, for example, CAR expression, CAR-T cell frequency, and CAR-T cell count.

### C. IRES-CO Constructs

These top four codon-optimized variants combined with the top IRES sequences were designed to be assessed in vitro for expression and anti-tumor functionality.

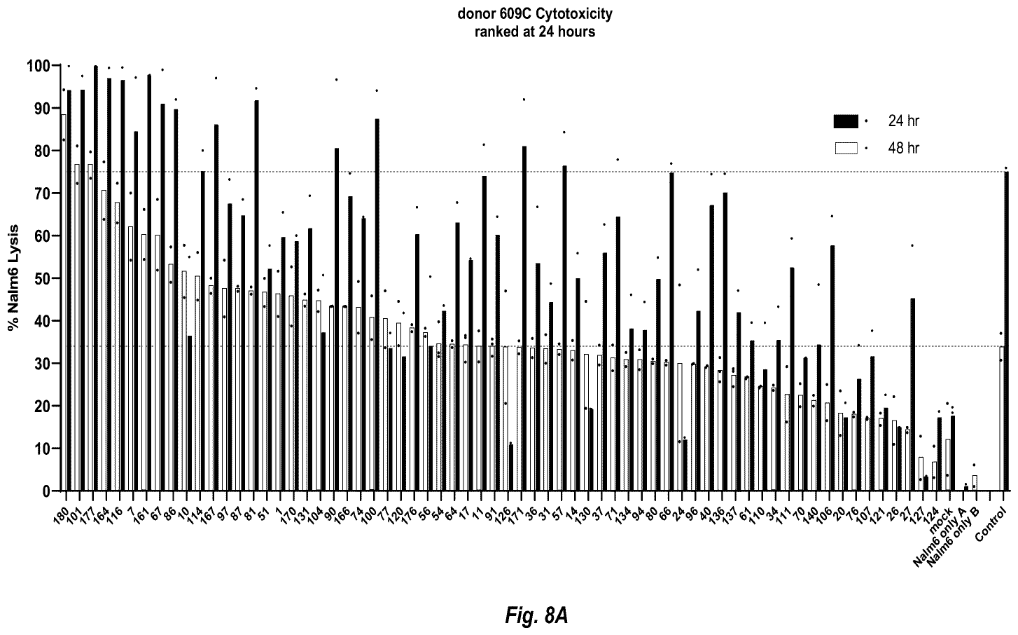

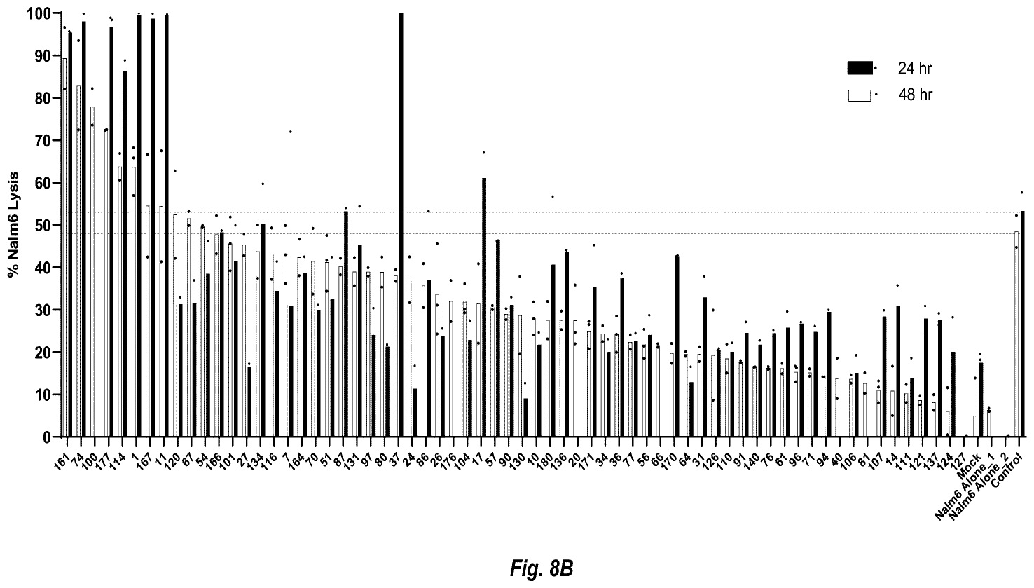

69 circular RNA constructs were designed comprising combinations of the 15 IRES sequences corresponding to SEQ ID NOs: 1-15 in Table 1A and the 5 anti-CD19 28-& codon-optimized expression sequences corresponding to the nucleotide sequences of SEQ ID NOs: 19-23 in Table 2A. The 69 constructs are described in Table 5 and in the examples, below. The constructs are referred to herein by the IRES/CO clone #of Table 5, for example, in FIGS. 8A and 8B.

TABLE 5
69 Experimental constructs

| IRES/CO | IRES | Codon |
|---|---|---|
| Clone # | SEQ ID NO | SEQ ID NO |
| 1 | 1 | 21 |
| 7 | 1 | 19 |
| 10 | 1 | 23 |
| 11 | 2 | 21 |
| 14 | 2 | 22 |
| 16 | 2 | 20 |
| 17 | 2 | 19 |
| 20 | 2 | 23 |
| 24 | 3 | 22 |
| 26 | 3 | 20 |
| 27 | 3 | 19 |
| 31 | 4 | 21 |
| 34 | 4 | 22 |
| 36 | 4 | 20 |
| 37 | 4 | 19 |
| 40 | 4 | 23 |
| 51 | 5 | 21 |
| 54 | 5 | 22 |
| 56 | 5 | 20 |
| 57 | 5 | 19 |
| 60 | 5 | 23 |
| 61 | 6 | 21 |
| 64 | 6 | 22 |
| 66 | 6 | 20 |
| 67 | 6 | 19 |
| 70 | 6 | 23 |
| 71 | 7 | 21 |
| 74 | 7 | 22 |
| 76 | 7 | 20 |
| 77 | 7 | 19 |
| 80 | 7 | 23 |
| 81 | 8 | 21 |
| 86 | 8 | 20 |
| 87 | 8 | 19 |
| 90 | 8 | 23 |
| 91 | 9 | 21 |
| 94 | 9 | 22 |
| 96 | 9 | 20 |
| 97 | 9 | 19 |
| 100 | 9 | 23 |
| 101 | 10 | 21 |
| 104 | 10 | 22 |
| 106 | 10 | 20 |
| 107 | 10 | 19 |
| 110 | 10 | 23 |
| 111 | 11 | 21 |
| 114 | 11 | 22 |
| 116 | 11 | 20 |
| 117 | 11 | 19 |
| 120 | 11 | 23 |
| 121 | 12 | 21 |
| 124 | 12 | 22 |
| 126 | 12 | 20 |
| 127 | 12 | 19 |
| 130 | 12 | 23 |
| 131 | 13 | 21 |
| 134 | 13 | 22 |
| 136 | 13 | 20 |
| 137 | 13 | 19 |
| 140 | 13 | 23 |
| 161 | 14 | 21 |
| 164 | 14 | 22 |
| 166 | 14 | 20 |
| 167 | 14 | 19 |
| 170 | 14 | 23 |
| 171 | 15 | 21 |
| 176 | 15 | 20 |
| 177 | 15 | 19 |
| 180 | 15 | 23 |

The full IRES-codon sequences for 12 of the 69 exemplary constructs of Table 5 are set forth in Table 6, below. As described in Examples 4-6, these 12 exemplary constructs generally exhibit high and durable expression and led to higher function of the CAR expressing T-cells.

TABLE 6
12 Exemplary construct sequences

| SEQ | | |
|---|---|---|
| ID | IRES/CO | |
| NO | Clone # | IRES + CO Sequence |
| 50 | 7 | TTAAAACAGCTCTGGGGTTGTTCCCACCCCAGAGGCCCACGTGGCGGCCA |
| | | GTACTCCGGTATTACGGTACCCTTGTACGCCTGTTTTATACTCCCTTCCCCT |
| | | GTAACTTAGAAGCATACAAACCAAGTTCAATAGAAGGGGGTACAAACCA |
| | | GTACCACCACGAACAAGCACTCCTGTTTCCCCGGTGACATTGCATAGACT |
| | | GTACCCACGGTTGAAAGCGATCGATCCGTTACCCGCTCCTGTACTTCGAG |
| | | AAGCCTAGTATCATCTTGGAATCTTCGATGCGTTGCGCTCAGCACTCAACC |
| | | CCAGAGTGTAGCTTAGGCTGATGAGTCTGGACGTCCCCCACCGGCGACGG |
| | | TGGTCCAGGCTGCGTTGGCGGCCTACCTGTGGCCCAAAGCCACAGGACGC |
| | | TAGTTGTGAACAAGGTGTGAAGAGCCTATTGAGCTACAAGAGAGTCCTCC |
| | | GGCCCCTGAATGCGGCTAATCCTAACCACGGAGCAGGCAGTTGCAAACCA |
| | | GCAACCGGCCTGTCGTAACGCGCAAGTCTGTGGCGGAACCGACTACTTTG |
| | | GGTGTCCGTGTTTCCTTTTATTTTTACAATGGCTGCTTATGGTGACAATCA |
| | | TAGATTGTTATCATAAAGCGACTTGGATTGGCCATCCGGTGAAAGTAAAA |
| | | CACATTGTTTACTTGTTTGTTGGATTCACTCCAATTAACACTTTTACTTACA |
| | | AACTCATTACAACAACTCTATTAATTAGAGATAAGCATCACAATGGCACT |
| | | GCCCGTCACCGCACTCCTGCTCCCACTGGCACTGCTGCTCCATGCAGCTCG |
| | | CCCCGATATCCAGATGACCCAGACCACCTCTAGCCTCAGCGCCTCTCTGG |
| | | GTGACCGCGTCACCATCTCTTGCCGGGCCAGCCAAGACATCTCTAAGTAC |
| | | CTGAACTGGTACCAGCAGAAACCTGACGGAACCGTGAAGCTGCTGATCTA |
| | | CCACACCAGTCGGCTGCATTCCGGGGTGCCTTCCAGGTTCAGCGGTTCCG |
| | | GCTCTGGGACCGATTATAGTCTCACCATCTCCAACCTCGAGCAGGAGGAC |
| | | ATCGCAACCTACTTCTGCCAGCAGGGGAACACCCTGCCCTACACCTTCGG |
| | | TGGCGGGACCAAGCTGGAGATCACTGGAGGTGGTGGCAGCGGAGGTGGA |
| | | GGATCAGGTGGAGGCGGTAGCGAGGTGAAGCTGCAGGAGTCCGGACCTG |
| | | GTCTGGTGGCCCCAAGCCAGTCCCTCAGCGTCACCTGCACAGTGTCCGGG |
| | | GTGTCCCTGCCTGACTACGGTGTCTCCTGGATCAGGCAACCACCCCGGAA |
| | | GGGTCTCGAGTGGCTGGGCGTCATCTGGGGCTCCGAGACCACCTACTACA |
| | | ACAGCGCTCTGAAGTCCCGGCTGACCATCATCAAAGACAACTCCAAGAGC |
| | | CAGGTGTTCTTGAAGATGAACTCCCTGCAAACCGATGACACCGCCATCTA |
| | | CTACTGCGCCAAGCACTACTACTATGGCGGTAGCTACGCCATGGATTATT |
| | | GGGGTCAGGGCACCAGTGTCACCGTCTCCTCCATCGAGGTGATGTACCCT |
| | | CCACCCTATCTGGACAACGAGAAGTCCAACGGCACCATCATCCACGTGAA |
| | | GGGCAAGCACCTGTGCCCTAGCCCTCTGTTCCCAGGACCCTCCAAGCCCT |
| | | TCTGGGTGCTGGTCGTGGTGGGAGGAGTCCTGGCCTGCTATTCCCTCCTCG |
| | | TCACCGTGGCATTTATCATCTTCTGGGTCCGGAGCAAGCGGTCACGCCTG |
| | | CTCCACTCCGACTACATGAACATGACTCCTCGCAGACCTGGACCCACCCG |
| | | GAAGCACTACCAGCCTTATGCCCCACCCCGCGACTTTGCCGCTTACCGCTC |
| | | TCGGGTCAAGTTCTCTCGGTCAGCAGACGCCCCTGCATACCAGCAGGGCC |
| | | AGAACCAGCTGTATAACGAGCTGAACCTCGGCAGACGGGAGGAGTACGA |
| | | TGTGCTGGACAAGAGGAGAGGCAGAGACCCCGAGATGGGTGGTAAGCCA |
| | | CGGCGCAAGAACCCACAGGAGGGCTTGTACAACGAACTGCAGAAGGACA |
| | | AGATGGCCGAGGCCTACAGCGAGATCGGCATGAAGGGAGAGAGGCGCAG |
| | | GGGCAAGGGTCACGACGGCCTGTACCAAGGGCTGTCCACCGCAACCAAG |
| | | GACACCTACGATGCCCTGCACATGCAGGCCCTCCCACCAAGG |
| 51 | 17 | TTTCCCCTGTTCGTAACTAAGTGTGTGCCCAATCTCCTCACTCCTGCTGGC |
| | | TTCACCGACCGGCAGTGTCCAAAATGCTAGGTGAATCCCCTCCCTTTCCTC |
| | | TGGGCTTCTGCCCAGCTTCCTCCCCCCAGCCTGACGTGACACAGGCTGTGC |
| | | AAAGACCCCGCGAAAGCTGCCAAAAGTGGCAATTGTGGGTCCCCCCTTTG |
| | | TAAAGGCGTCGAGTCTTTCTCCCTCAAGGCTAGACCCGTCAGTGAATTCT |
| | | GTCGGGCAACTAGTGACGCCACTGCACGCCTCTGACCTCGGCCGCGGAGT |
| | | GCTGCCCCCCAAGTCGTGCCCCTGACCACAAGTTGTGCTGTCTGGCAAAC |
| | | ATTGTCTGTGAGAATGTTCCGCTGTGGCTGCCAAGCCTGGCAACAGGCTG |
| | | CCCCAGTGTGCGTAGTTCTCATCCAGACTTCGGTCTGGCAACTTGCTGTTA |
| | | AGACACGGCGTAAGGGGCGTGTGCCAACGCCCTGGAACGAGTGTCCACT |
| | | CTAATACCCCGAGGAATGCTACGCAGGTACCCCTGGTTCGCCAGGGATCT |
| | | GAGCGTAGGCTAATTGTCTAAGGGTATTTTCATTTCCCATTCTTTCTTTCTT |
| | | GTTCATAATGGCACTGCCCGTCACCGCACTCCTGCTCCCACTGGCACTGCT |
| | | GCTCCATGCAGCTCGCCCCGATATCCAGATGACCCAGACCACCTCTAGCC |
| | | TCAGCGCCTCTCTGGGTGACCGCGTCACCATCTCTTGCCGGGCCAGCCAA |
| | | GACATCTCTAAGTACCTGAACTGGTACCAGCAGAAACCTGACGGAACCGT |
| | | GAAGCTGCTGATCTACCACACCAGTCGGCTGCATTCCGGGGTGCCTTCCA |
| | | GGTTCAGCGGTTCCGGCTCTGGGACCGATTATAGTCTCACCATCTCCAACC |
| | | TCGAGCAGGAGGACATCGCAACCTACTTCTGCCAGCAGGGGAACACCCTG |
| | | CCCTACACCTTCGGTGGCGGGACCAAGCTGGAGATCACTGGAGGTGGTGG |
| | | CAGCGGAGGTGGAGGATCAGGTGGAGGCGGTAGCGAGGTGAAGCTGCAG |
| | | GAGTCCGGACCTGGTCTGGTGGCCCCAAGCCAGTCCCTCAGCGTCACCTG |
| | | CACAGTGTCCGGGGTGTCCCTGCCTGACTACGGTGTCTCCTGGATCAGGC |
| | | AACCACCCCGGAAGGGTCTCGAGTGGCTGGGCGTCATCTGGGGCTCCGAG |
| | | ACCACCTACTACAACAGCGCTCTGAAGTCCCGGCTGACCATCATCAAAGA |
| | | CAACTCCAAGAGCCAGGTGTTCTTGAAGATGAACTCCCTGCAAACCGATG |
| | | ACACCGCCATCTACTACTGCGCCAAGCACTACTACTATGGCGGTAGCTAC |
| | | GCCATGGATTATTGGGGTCAGGGCACCAGTGTCACCGTCTCCTCCATCGA |
| | | GGTGATGTACCCTCCACCCTATCTGGACAACGAGAAGTCCAACGGCACCA |
| | | TCATCCACGTGAAGGGCAAGCACCTGTGCCCTAGCCCTCTGTTCCCAGGA |
| | | CCCTCCAAGCCCTTCTGGGTGCTGGTCGTGGTGGGAGGAGTCCTGGCCTG |
| | | CTATTCCCTCCTCGTCACCGTGGCATTTATCATCTTCTGGGTCCGGAGCAA |
| | | GCGGTCACGCCTGCTCCACTCCGACTACATGAACATGACTCCTCGCAGAC |
| | | CTGGACCCACCCGGAAGCACTACCAGCCTTATGCCCCACCCCGCGACTTT |
| | | GCCGCTTACCGCTCTCGGGTCAAGTTCTCTCGGTCAGCAGACGCCCCTGC |
| | | ATACCAGCAGGGCCAGAACCAGCTGTATAACGAGCTGAACCTCGGCAGA |
| | | CGGGAGGAGTACGATGTGCTGGACAAGAGGAGAGGCAGAGACCCCGAGA |
| | | TGGGTGGTAAGCCACGGCGCAAGAACCCACAGGAGGGCTTGTACAACGA |
| | | ACTGCAGAAGGACAAGATGGCCGAGGCCTACAGCGAGATCGGCATGAAG |
| | | GGAGAGAGGCGCAGGGGCAAGGGTCACGACGGCCTGTACCAAGGGCTGT |
| | | CCACCGCAACCAAGGACACCTACGATGCCCTGCACATGCAGGCCCTCCCA |
| | | CCAAGG |
| 52 | 37 | TTAAAACAGCGGATGGGTATCCCACCATCCGGCCCACTGGGTGTAGTACT |
| | | CTGGTACATTGTACCTTTGTACGCCTGTTTTCCCCCTCTTGTACCCGCCCTT |
| | | CAAGCTCCTTGCCCAAGTAACGTTAGAAGTTTGAACATTGGTACAATAGG |
| | | AAGCATCACATCCAGTGGTGTACTGTACAAACACTTCTGTTGCCCCGGAG |
| | | CGAGGTATAGATGGTCCCCACCGTCAAAAGCCTTTAACCGTTATCCGCCA |
| | | ATCAACTACGTAATGGCTAGTAGCACCTTGGATTTAAGTTGGCGTTCGAT |
| | | CAGGTGGTAACCCCCACTAGTTTGGTCGATGAGGCTAGGAATTCCCCACG |
| | | GGTGACCGTGTCCTAGCCTGCGTGGCGGCCAACCCAGCATCCGCTGGGAC |
| | | GCCAATTTAATGACATGGTGTGAAGACCTGCATGTGCTTGATTGTGAGTC |
| | | CTCCGGCCCCTGAATGCGGCTAACCCTAACCCCGGAGCCTTGCAGCACAA |
| | | TCCAGTGTTGTTAAGGTCGTAATGAGCAATTCTGGGATGGGACCGACTAC |
| | | TTTGGGTGTCCGTGTTTCTTATTTTTCTTGAATTTTTCTTATGGTCACAGCA |
| | | TATATACATTATATACTGTGATCATGGCACTGCCCGTCACCGCACTCCTGC |
| | | TCCCACTGGCACTGCTGCTCCATGCAGCTCGCCCCGATATCCAGATGACC |
| | | CAGACCACCTCTAGCCTCAGCGCCTCTCTGGGTGACCGCGTCACCATCTCT |
| | | TGCCGGGCCAGCCAAGACATCTCTAAGTACCTGAACTGGTACCAGCAGAA |
| | | ACCTGACGGAACCGTGAAGCTGCTGATCTACCACACCAGTCGGCTGCATT |
| | | CCGGGGTGCCTTCCAGGTTCAGCGGTTCCGGCTCTGGGACCGATTATAGT |
| | | CTCACCATCTCCAACCTCGAGCAGGAGGACATCGCAACCTACTTCTGCCA |
| | | GCAGGGGAACACCCTGCCCTACACCTTCGGTGGCGGGACCAAGCTGGAG |
| | | ATCACTGGAGGTGGTGGCAGCGGAGGTGGAGGATCAGGTGGAGGCGGTA |
| | | GCGAGGTGAAGCTGCAGGAGTCCGGACCTGGTCTGGTGGCCCCAAGCCA |
| | | GTCCCTCAGCGTCACCTGCACAGTGTCCGGGGTGTCCCTGCCTGACTACG |
| | | GTGTCTCCTGGATCAGGCAACCACCCCGGAAGGGTCTCGAGTGGCTGGGC |
| | | GTCATCTGGGGCTCCGAGACCACCTACTACAACAGCGCTCTGAAGTCCCG |
| | | GCTGACCATCATCAAAGACAACTCCAAGAGCCAGGTGTTCTTGAAGATGA |
| | | ACTCCCTGCAAACCGATGACACCGCCATCTACTACTGCGCCAAGCACTAC |
| | | TACTATGGCGGTAGCTACGCCATGGATTATTGGGGTCAGGGCACCAGTGT |
| | | CACCGTCTCCTCCATCGAGGTGATGTACCCTCCACCCTATCTGGACAACG |
| | | AGAAGTCCAACGGCACCATCATCCACGTGAAGGGCAAGCACCTGTGCCCT |
| | | AGCCCTCTGTTCCCAGGACCCTCCAAGCCCTTCTGGGTGCTGGTCGTGGTG |
| | | GGAGGAGTCCTGGCCTGCTATTCCCTCCTCGTCACCGTGGCATTTATCATC |
| | | TTCTGGGTCCGGAGCAAGCGGTCACGCCTGCTCCACTCCGACTACATGAA |
| | | CATGACTCCTCGCAGACCTGGACCCACCCGGAAGCACTACCAGCCTTATG |
| | | CCCCACCCCGCGACTTTGCCGCTTACCGCTCTCGGGTCAAGTTCTCTCGGT |
| | | CAGCAGACGCCCCTGCATACCAGCAGGGCCAGAACCAGCTGTATAACGA |
| | | GCTGAACCTCGGCAGACGGGAGGAGTACGATGTGCTGGACAAGAGGAGA |
| | | GGCAGAGACCCCGAGATGGGTGGTAAGCCACGGCGCAAGAACCCACAGG |
| | | AGGGCTTGTACAACGAACTGCAGAAGGACAAGATGGCCGAGGCCTACAG |
| | | CGAGATCGGCATGAAGGGAGAGAGGCGCAGGGGCAAGGGTCACGACGG |
| | | CCTGTACCAAGGGCTGTCCACCGCAACCAAGGACACCTACGATGCCCTGC |
| | | ACATGCAGGCCCTCCCACCAAGG |
| 53 | 67 | TTCAAACAGCCTGGGGGTTGTACCCACCCCTGGGGCCCACGTGGCGCTAG |
| | | TACTCTGGTACGTTAGTACCTTTGTACGCCTGTTTTCCCCTCCCTTAAACA |
| | | AATTAAGATTACCACTACTGAGGGGAGTAGTCCGACTCCGCTCCGGTACT |
| | | GCCGCACCAGTACTCCGGTACACTTAGTACCCTAGTACGGAGTAGATGGT |
| | | ATCCCCACCCCGCAACTTAGAAGCATGCAAACAAACCGACCAATAGGCG |
| | | CACGATATCCAGTCGTGTTTCGGTCAAGCACTTCTGTCTCCCCGGTCCGAA |
| | | AGGATCGTTACCCGCCCGACCCACTACGAGAAGCCCAGTAACTGGCCAAG |
| | | TGATTGCGAAGTTGCGCTCAGCCACAACCCCAGTGGTAGCTCTGGAAGAT |
| | | GGGGCTCGCGTCTCCCCCGTGGTGACACGGTCGCTTGCCCGCGTGTGCTTC |
| | | CGGGTTCGGCCTACGCCGTTCACTTCAATGTCACGTAACCAGCCAAGAGC |
| | | CTATTGTGCTGGGACGGTTTTCCTCCGGGGCCGTGAATGCTGCTAATCCCA |
| | | ACCTCCGAGCGTGTGCGCACAACCCAGTGTTGCTACGTCGTAATGCGTAA |
| | | GTTGGAGGCGGAACAGACTACTTTCGGTACCCCGTGTTTCCTTTAAATTTT |
| | | ATTCATTATTTTATGGTGACAATTGCTGAGATCTGCGAATTAGCGACTCTG |
| | | CCGTTGAATATTGCTCTGTACTATTTGGTTGCATTCCACAAAACCTCTGAC |
| | | ATCCCCAGTACATACATTACTTTACTTGTTTACCTCAATCTAAAGCACAAG |
| | | CTAGATAATACAAAATGGCACTGCCCGTCACCGCACTCCTGCTCCCACTG |
| | | GCACTGCTGCTCCATGCAGCTCGCCCCGATATCCAGATGACCCAGACCAC |
| | | CTCTAGCCTCAGCGCCTCTCTGGGTGACCGCGTCACCATCTCTTGCCGGGC |
| | | CAGCCAAGACATCTCTAAGTACCTGAACTGGTACCAGCAGAAACCTGACG |
| | | GAACCGTGAAGCTGCTGATCTACCACACCAGTCGGCTGCATTCCGGGGTG |
| | | CCTTCCAGGTTCAGCGGTTCCGGCTCTGGGACCGATTATAGTCTCACCATC |
| | | TCCAACCTCGAGCAGGAGGACATCGCAACCTACTTCTGCCAGCAGGGGAA |
| | | CACCCTGCCCTACACCTTCGGTGGGGGACCAAGCTGGAGATCACTGGAG |
| | | GTGGTGGCAGCGGAGGTGGAGGATCAGGTGGAGGCGGTAGCGAGGTGAA |
| | | GCTGCAGGAGTCCGGACCTGGTCTGGTGGCCCCAAGCCAGTCCCTCAGCG |
| | | TCACCTGCACAGTGTCCGGGGTGTCCCTGCCTGACTACGGTGTCTCCTGGA |
| | | TCAGGCAACCACCCCGGAAGGGTCTCGAGTGGCTGGGCGTCATCTGGGGC |
| | | TCCGAGACCACCTACTACAACAGCGCTCTGAAGTCCCGGCTGACCATCAT |
| | | CAAAGACAACTCCAAGAGCCAGGTGTTCTTGAAGATGAACTCCCTGCAAA |
| | | CCGATGACACCGCCATCTACTACTGCGCCAAGCACTACTACTATGGCGGT |
| | | AGCTACGCCATGGATTATTGGGGTCAGGGCACCAGTGTCACCGTCTCCTC |
| | | CATCGAGGTGATGTACCCTCCACCCTATCTGGACAACGAGAAGTCCAACG |
| | | GCACCATCATCCACGTGAAGGGCAAGCACCTGTGCCCTAGCCCTCTGTTC |
| | | CCAGGACCCTCCAAGCCCTTCTGGGTGCTGGTCGTGGTGGGAGGAGTCCT |
| | | GGCCTGCTATTCCCTCCTCGTCACCGTGGCATTTATCATCTTCTGGGTCCG |
| | | GAGCAAGCGGTCACGCCTGCTCCACTCCGACTACATGAACATGACTCCTC |
| | | GCAGACCTGGACCCACCCGGAAGCACTACCAGCCTTATGCCCCACCCCGC |
| | | GACTTTGCCGCTTACCGCTCTCGGGTCAAGTTCTCTCGGTCAGCAGACGCC |
| | | CCTGCATACCAGCAGGGCCAGAACCAGCTGTATAACGAGCTGAACCTCGG |
| | | CAGACGGGAGGAGTACGATGTGCTGGACAAGAGGAGAGGCAGAGACCCC |
| | | GAGATGGGTGGTAAGCCACGGCGCAAGAACCCACAGGAGGGCTTGTACA |
| | | ACGAACTGCAGAAGGACAAGATGGCCGAGGCCTACAGCGAGATCGGCAT |
| | | GAAGGGAGAGAGGCGCAGGGGCAAGGGTCACGACGGCCTGTACCAAGG |
| | | GCTGTCCACCGCAACCAAGGACACCTACGATGCCCTGCACATGCAGGCCC |
| | | TCCCACCAAGG |
| 54 | 86 | CCCCCCTCCCCCCCTTCCCTTCCCTTTGCAACGCAACAATTGTAAGTGCCC |
| | | TCACCTGTCAATTGGGACCACCACTTTCAGTGACCCCATGCGAAGTGCTG |
| | | AGAGAAAGGAAGCTTTCTTACCCTTCATTTGTGAACCCACTGGTCTAAGC |
| | | CGCTTGGAATACGATGAGTGGAAAAGTTCATTCTTAATGGAGTGAAACAT |
| | | GCTTAAATTTCCAGCTCGTGCTGGTCTTTCCAGTACGGGGCGGCCCTGTCT |
| | | GGCCGTAATTCTTCAGAGTGTCACGCCACACTTGTGGATCTCACGTGCCA |
| | | CATGACAGCGCTACAGCTGGAACTGGGTGCTTGGTGCCCATGGAGTAACA |
| | | GCGAAAAGTGTTAGATCAAGCCTTGCTTGGGCTATGAGCCTGCGGAACAA |
| | | CAACTGGTAACAGTTGCCTCAGGGGCCGAAAGCCACGGTGTTAACAGCAC |
| | | CCTCATAGTTTGATCCACCTCAGGGTGGTGATGTTTAGCAGTTAGTAGTTG |
| | | CCAATCTGTGTTCACTGAAATCTCGGCATACCGTGTAGTGTACAGGGGTG |
| | | AAGGATGCCCAGAAGGTACCCGTAGGTAACCTTAAGAGACTATGGATCTG |
| | | ATCTGGGGCCTTGTCCGGAGTGCTTTACACACGGCTCAAGGTTAAAAAAC |
| | | GTCTAGCCCCACAGAGCCCGAGGGATTCGGGTTTTCCCTTTAAAAACCCG |
| | | ACTAGAGCTTATGGTGACAATTATTGCTGTTCAGACGAACAGTGTAATTG |
| | | TTGTCTATTCACAGCAGTTCTATCAGAGCTTTTCCCACAACGGATCTTCTT |
| | | GGCAAGCAAATACAGCAGGAGTCAATATGGCACTCCCAGTCACCGCACTT |
| | | CTGCTGCCTCTCGCCCTGCTGCTCCATGCAGCCAGACCCGACATCCAGAT |
| | | GACCCAAACCACCAGCTCCCTGTCCGCTTCCCTGGGTGACCGGGTGACTA |
| | | TCTCTTGCCGGGCCTCCCAAGACATCTCCAAGTACCTGAACTGGTATCAG |
| | | CAAAAGCCTGACGGCACCGTCAAGCTCCTCATCTACCATACCTCCAGACT |
| | | GCACTCCGGGGTGCCTAGCAGGTTCAGCGGAAGTGGGAGCGGCACCGAC |
| | | TACAGCCTCACCATCTCCAACCTGGAGCAGGAGGACATCGCCACCTACTT |
| | | CTGCCAGCAGGGGAACACACTGCCCTACACCTTCGGCGGTGGCACCAAGC |
| | | TGGAGATCACAGGTGGCGGAGGTTCCGGAGGAGGAGGTAGTGGAGGTGG |
| | | AGGCAGCGAGGTGAAGCTCCAGGAATCCGGACCAGGTCTGGTGGCTCCC |
| | | AGCCAGTCCCTCAGCGTGACCTGCACCGTGAGCGGCGTGTCTCTTCCCGA |
| | | TTACGGAGTGTCCTGGATCAGACAGCCACCCCGGAAGGGTCTGGAGTGGC |
| | | TGGGAGTGATCTGGGGTTCCGAGACCACATACTACAACTCAGCCCTCAAG |
| | | AGCCGGCTCACCATCATCAAGGATAACTCCAAGTCCCAGGTCTTCCTGAA |
| | | GATGAACTCTCTCCAGACCGACGACACCGCCATCTACTACTGCGCCAAGC |
| | | ACTACTACTACGGGGGTCCTACGCCATGGACTACTGGGGTCAGGGAACC |
| | | TCCGTCACCGTCAGCTCTATCGAGGTGATGTACCCTCCTCCCTACCTCGAC |
| | | AACGAGAAGAGCAACGGCACCATCATCCATGTGAAGGGGAAGCATCTCT |
| | | GCCCCTCACCCCTGTTCCCCGGACCATCCAAGCCATTCTGGGTGCTGGTGG |
| | | TTGTTGGTGGGGTCCTGGCTTGCTACTCACTCCTGGTCACCGTCGCCTTCA |
| | | TCATCTTCTGGGTGCGGTCAAAGAGGTCCCGGCTCTTGCACTCCGATTACA |
| | | TGAACATGACTCCAAGGAGGCCTGGTCCCACACGGAAGCACTACCAACC |
| | | ATATGCCCCACCACGCGACTTCGCTGCTTACCGGAGCCGGGTCAAGTTCA |
| | | GTCGGAGTGCAGACGCCCCAGCCTACCAGCAGGGCCAGAACCAACTCTA |
| | | CAACGAGCTTAATCTGGGTCGCCGGGAGGAGTATGACGTGCTCGATAAGA |
| | | GAAGGGGCCGGGATCCTGAGATGGGGGTAAGCCCAGACGGAAGAACCC |
| | | TCAGGAGGGGTTGTATAATGAGCTCCAGAAGGACAAGATGGCCGAGGCA |
| | | TACTCCGAGATCGGCATGAAAGGTGAGCGGAGGAGAGGCAAGGGGCATG |
| | | ACGGCCTGTACCAGGGGCTCAGCACAGCCACCAAGGATACCTATGACGC |
| | | ACTCCACATGCAGGCACTGCCTCCACGG |
| 55 | 87 | CCCCCCTCCCCCCCTTCCCTTCCCTTTGCAACGCAACAATTGTAAGTGCCC |
| | | TCACCTGTCAATTGGGACCACCACTTTCAGTGACCCCATGCGAAGTGCTG |
| | | AGAGAAAGGAAGCTTTCTTACCCTTCATTTGTGAACCCACTGGTCTAAGC |
| | | CGCTTGGAATACGATGAGTGGAAAAGTTCATTCTTAATGGAGTGAAACAT |
| | | GCTTAAATTTCCAGCTCGTGCTGGTCTTTCCAGTACGGGGCGGCCCTGTCT |
| | | GGCCGTAATTCTTCAGAGTGTCACGCCACACTTGTGGATCTCACGTGCCA |
| | | CATGACAGCGCTACAGCTGGAACTGGGTGCTTGGTGCCCATGGAGTAACA |
| | | GCGAAAAGTGTTAGATCAAGCCTTGCTTGGGCTATGAGCCTGCGGAACAA |
| | | CAACTGGTAACAGTTGCCTCAGGGGCCGAAAGCCACGGTGTTAACAGCAC |
| | | CCTCATAGTTTGATCCACCTCAGGGTGGTGATGTTTAGCAGTTAGTAGTTG |
| | | CCAATCTGTGTTCACTGAAATCTCGGCATACCGTGTAGTGTACAGGGGTG |
| | | AAGGATGCCCAGAAGGTACCOGTAGGTAACCTTAAGAGACTATGGATCTG |
| | | ATCTGGGGCCTTGTCCGGAGTGCTTTACACACGGCTCAAGGTTAAAAAAC |
| | | GTCTAGCCCCACAGAGCCCGAGGGATTCGGGTTTTCCCTTTAAAAACCCG |
| | | ACTAGAGCTTATGGTGACAATTATTGCTGTTCAGACGAACAGTGTAATTG |
| | | TTGTCTATTCACAGCAGTTCTATCAGAGCTTTTCCCACAACGGATCTTCTT |
| | | GGCAAGCAAATACAGCAGGAGTCAATATGGCACTGCCCGTCACCGCACTC |
| | | CTGCTCCCACTGGCACTGCTGCTCCATGCAGCTCGCCCCGATATCCAGATG |
| | | ACCCAGACCACCTCTAGCCTCAGCGCCTCTCTGGGTGACCGCGTCACCAT |
| | | CTCTTGCCGGGCCAGCCAAGACATCTCTAAGTACCTGAACTGGTACCAGC |
| | | AGAAACCTGACGGAACCGTGAAGCTGCTGATCTACCACACCAGTCGGCTG |
| | | CATTCCGGGGTGCCTTCCAGGTTCAGCGGTTCCGGCTCTGGGACCGATTAT |
| | | AGTCTCACCATCTCCAACCTCGAGCAGGAGGACATCGCAACCTACTTCTG |
| | | CCAGCAGGGGAACACCCTGCCCTACACCTTCGGTGGCGGGACCAAGCTGG |
| | | AGATCACTGGAGGTGGTGGCAGCGGAGGTGGAGGATCAGGTGGAGGCGG |
| | | TAGCGAGGTGAAGCTGCAGGAGTCCGGACCTGGTCTGGTGGCCCCAAGCC |
| | | AGTCCCTCAGCGTCACCTGCACAGTGTCCGGGGTGTCCCTGCCTGACTAC |
| | | GGTGTCTCCTGGATCAGGCAACCACCCCGGAAGGGTCTCGAGTGGCTGGG |
| | | CGTCATCTGGGGCTCCGAGACCACCTACTACAACAGCGCTCTGAAGTCCC |
| | | GGCTGACCATCATCAAAGACAACTCCAAGAGCCAGGTGTTCTTGAAGATG |
| | | AACTCCCTGCAAACCGATGACACCGCCATCTACTACTGCGCCAAGCACTA |
| | | CTACTATGGCGGTAGCTACGCCATGGATTATTGGGGTCAGGGCACCAGTG |
| | | TCACCGTCTCCTCCATCGAGGTGATGTACCCTCCACCCTATCTGGACAACG |
| | | AGAAGTCCAACGGCACCATCATCCACGTGAAGGGCAAGCACCTGTGCCCT |
| | | AGCCCTCTGTTCCCAGGACCCTCCAAGCCCTTCTGGGTGCTGGTCGTGGTG |
| | | GGAGGAGTCCTGGCCTGCTATTCCCTCCTCGTCACCGTGGCATTTATCATC |
| | | TTCTGGGTCCGGAGCAAGCGGTCACGCCTGCTCCACTCCGACTACATGAA |
| | | CATGACTCCTCGCAGACCTGGACCCACCCGGAAGCACTACCAGCCTTATG |
| | | CCCCACCCCGCGACTTTGCCGCTTACCGCTCTCGGGTCAAGTTCTCTCGGT |
| | | CAGCAGACGCCCCTGCATACCAGCAGGGCCAGAACCAGCTGTATAACGA |
| | | GCTGAACCTCGGCAGACGGGAGGAGTACGATGTGCTGGACAAGAGGAGA |
| | | GGCAGAGACCCCGAGATGGGTGGTAAGCCACGGCGCAAGAACCCACAGG |
| | | AGGGCTTGTACAACGAACTGCAGAAGGACAAGATGGCCGAGGCCTACAG |
| | | CGAGATCGGCATGAAGGGAGAGAGGCGCAGGGGCAAGGGTCACGACGG |
| | | CCTGTACCAAGGGCTGTCCACCGCAACCAAGGACACCTACGATGCCCTGC |
| | | ACATGCAGGCCCTCCCACCAAGG |
| 56 | 97 | CACTACGTTACGGTTCCCGCCCGGGACAACTGGTACCCCATTAGGCTACA |
| | | ACATGGCTGAAAAGGGTATTGGGTCCCCCCGGATTGTGTCCGTTCGTAGT |
| | | GTGTGTAACGTGGTTTACCATCTCCACTAACATTGGACTAAGCATTTCATC |
| | | TTTCCTCCCCGATTGTGTACTCACTTGGCTAACGCTGGGTGGTCGCGGTTG |
| | | GGTCCTTGATTTACTTTTTCTCGTCTAAGCATTCCGACTGTCCTCCCCGATT |
| | | ATGTGCTCATTCAGTTAACTGCTGGGTGGTCATGACTAACATCGAGGAAC |
| | | CTTCTGTCCACGCTTACTTTGAGCTCCGGTCGCTTGACGCTTGTAGGGCGA |
| | | TAGGGTTATCTTCCTGACAACATCTTTATTCTACCTCCATAGGCTCTATCT |
| | | ATGGAGACGGAGTGTGGCACCCGTCCCTTCTTTGGGAGCTTCGGTAGTGA |
| | | CGCCCTTTGTCACTCTCGCCAGCCGAGGCATGCCTGGTGCCAGGTAGCAA |
| | | AGAAAGCATATGTTTAAGGACTTGACTGATTTAGCGCAAGAGTTTGTAGC |
| | | GATGTCCATAGTGTCTGCGGATTCCCCACACGGCGACGTGTGCCGCGGAG |
| | | GCCAAAAGCCACGGTGTTCACAGCACCCCTATGGATGCCCACAGACCCCA |
| | | GTGGGCACTCTTGTTGCCGGACTTTCAGGAAATTAGGCATAGGCTCTTCTC |
| | | AAACTCCTGGCATTGGACTAGGTAAGAATGCCCCGGAGGTACCCCAGTAC |
| | | TCCTTCGGGAGTCTGGGATCTGACCGGGGGCCCCACAAACATGCTTTACG |
| | | TGTTTCGTGCGGTCAAAAATTGTCTAACTAGTCCCAACCTTGAACAAGGG |
| | | ATTGTTCTTTCCTTTTTATTACTGAGACTGGCCTATGGTGACAACAGAGAT |
| | | TGACTGTGAATACAGTTATTTTCTGGTGTTTATCATTTGGTTTTTCTCCGTG |
| | | CTCTTTTACCTTTGTGGTATTTGTTCTTTAGATAGGCAAAATGGCACTGCC |
| | | CGTCACCGCACTCCTGCTCCCACTGGCACTGCTGCTCCATGCAGCTCGCCC |
| | | CGATATCCAGATGACCCAGACCACCTCTAGCCTCAGCGCCTCTCTGGGTG |
| | | ACCGCGTCACCATCTCTTGCCGGGCCAGCCAAGACATCTCTAAGTACCTG |
| | | AACTGGTACCAGCAGAAACCTGACGGAACCGTGAAGCTGCTGATCTACCA |
| | | CACCAGTCGGCTGCATTCCGGGGTGCCTTCCAGGTTCAGCGGTTCCGGCT |
| | | CTGGGACCGATTATAGTCTCACCATCTCCAACCTCGAGCAGGAGGACATC |
| | | GCAACCTACTTCTGCCAGCAGGGGAACACCCTGCCCTACACCTTCGGTGG |
| | | CGGGACCAAGCTGGAGATCACTGGAGGTGGTGGCAGCGGAGGTGGAGGA |
| | | TCAGGTGGAGGCGGTAGCGAGGTGAAGCTGCAGGAGTCCGGACCTGGTC |
| | | TGGTGGCCCCAAGCCAGTCCCTCAGCGTCACCTGCACAGTGTCCGGGGTG |
| | | TCCCTGCCTGACTACGGTGTCTCCTGGATCAGGCAACCACCCCGGAAGGG |
| | | TCTCGAGTGGCTGGGCGTCATCTGGGGCTCCGAGACCACCTACTACAACA |
| | | GCGCTCTGAAGTCCCGGCTGACCATCATCAAAGACAACTCCAAGAGCCAG |
| | | GTGTTCTTGAAGATGAACTCCCTGCAAACCGATGACACCGCCATCTACTA |
| | | CTGCGCCAAGCACTACTACTATGGCGGTAGCTACGCCATGGATTATTGGG |
| | | GTCAGGGCACCAGTGTCACCGTCTCCTCCATCGAGGTGATGTACCCTCCA |
| | | CCCTATCTGGACAACGAGAAGTCCAACGGCACCATCATCCACGTGAAGGG |
| | | CAAGCACCTGTGCCCTAGCCCTCTGTTCCCAGGACCCTCCAAGCCCTTCTG |
| | | GGTGCTGGTCGTGGTGGGAGGAGTCCTGGCCTGCTATTCCCTCCTCGTCAC |
| | | CGTGGCATTTATCATCTTCTGGGTCCGGAGCAAGCGGTCACGCCTGCTCC |
| | | ACTCCGACTACATGAACATGACTCCTCGCAGACCTGGACCCACCCGGAAG |
| | | CACTACCAGCCTTATGCCCCACCCCGCGACTTTGCCGCTTACCGCTCTCGG |
| | | GTCAAGTTCTCTCGGTCAGCAGACGCCCCTGCATACCAGCAGGGCCAGAA |
| | | CCAGCTGTATAACGAGCTGAACCTCGGCAGACGGGAGGAGTACGATGTG |
| | | CTGGACAAGAGGAGAGGCAGAGACCCCGAGATGGGTGGTAAGCCACGGC |
| | | GCAAGAACCCACAGGAGGGCTTGTACAACGAACTGCAGAAGGACAAGAT |
| | | GGCCGAGGCCTACAGCGAGATCGGCATGAAGGGAGAGAGGCGCAGGGGC |
| | | AAGGGTCACGACGGCCTGTACCAAGGGCTGTCCACCGCAACCAAGGACA |
| | | CCTACGATGCCCTGCACATGCAGGCCCTCCCACCAAGG |
| 57 | 161 | TCACCCTCTTTTCCGGTGGTCCGGACCCAGACCACCGTTACTCCATTCAGC |
| | | TTCTTCGGAACCTGTTTGGAGGAATTAAACGGGCACCCACCCACCTTCAC |
| | | CCCCTTTTCGTAACTAAGTGTGTGCCCAATCTCATGACTCCTGCTGACTTC |
| | | ACCGACCAGCAGTGTCCAAAACGCTAGGTGAATTTCCTTCCTCCCCCTCTG |
| | | GGCTTCTGCCCAGCTCCCTCCCTCCAGCCTGACGTGCCACAGGCTGTGCA |
| | | AAGACCCCGCGAAAGCTGCCAAAAGTGGCAATTGTGGGTCCCCCCTTTGT |
| | | AAAGGCGTCGAGTCTTTCTCCCTTAAGGCTAGACCCGTCAGTGAATTCTGT |
| | | CGGGCAACTAGTGACGCCACTGCATGCCTCCGACCTCGGCCGCGGAGTGC |
| | | TGCCCCCCAAGTCGTGCCCCTGACTACAAGTIGTGCTGTCTGGCAAACATT |
| | | GTCTGTGAGAATGTTCCGCTGTGGCTGCCAAGCCTGGTAACAGGCTGCCC |
| | | CAGTGTGCGTAGTTCTCATCCAGACTTCGGTCTGGCAACTTGCTGTTAAGA |
| | | CACGGCGTAAGGGGCGTGTGCCAACGCCCTGGAACGAGTGTCCACTCTAA |
| | | TACCCCGAGGAATGCTACGCAGGTACCCCTGGCTCCCCAGGGATCTGAGC |
| | | GTAGGCTAATTGTCTAAGGGTATTTTCATTTCCCACTCTTTCTTTCTTGTTC |
| | | ATAATGGCTCTGCCTGTGACAGCTCTGCTGCTGCCTCTGGCTCTGCTTCTG |
| | | CATGCCGCCAGACCTGACATCCAGATGACCCAGACAACCAGCAGCCTGTC |
| | | TGCCAGCCTGGGCGATAGAGTGACCATCAGCTGTAGAGCCAGCCAGGAC |
| | | ATCAGCAAGTACCTGAACTGGTATCAGCAGAAACCCGACGGCACCGTGA |
| | | AGCTGCTGATCTACCACACCAGCAGACTGCACAGCGGCGTGCCAAGCAG |
| | | ATTTTCTGGCAGCGGCTCTGGCACCGACTACAGCCTGACAATCAGCAACC |
| | | TGGAACAAGAGGATATCGCTACCTACTTCTGCCAGCAAGGCAACACCCTG |
| | | CCTTACACCTTTGGCGGAGGCACCAAGCTGGAAATCACAGGCGGCGGAG |
| | | GAAGCGGAGGCGGAGGATCTGGTGGTGGTGGATCTGAAGTGAAACTGCA |
| | | AGAGTCTGGCCCTGGCCTGGTGGCCCCATCTCAATCTCTGAGCGTGACCT |
| | | GTACCGTCAGCGGAGTGTCCCTGCCTGATTATGGCGTGTCCTGGATCCGG |
| | | CAGCCTCCTAGAAAAGGCCTGGAATGGCTGGGCGTGATCTGGGGCAGCG |
| | | AGACAACCTACTACAACAGCGCCCTGAAGTCCCGGCTGACCATCATCAAG |
| | | GACAACTCCAAGAGCCAGGTGTTCCTGAAGATGAACAGCCTGCAGACCG |
| | | ACGACACCGCCATCTACTATTGCGCCAAGCACTACTACTACGGCGGCAGC |
| | | TACGCCATGGATTATTGGGGCCAGGGCACCAGCGTGACCGTGTCTAGCAT |
| | | CGAAGTGATGTACCCTCCACCTTACCTGGACAACGAGAAGTCCAACGGCA |
| | | CCATCATCCACGTGAAGGGCAAGCACCTGTGTCCTTCTCCACTGTTCCCCG |
| | | GACCTAGCAAGCCTTTCTGGGTGCTCGTTGTTGTTGGCGGCGTGCTGGCCT |
| | | GTTACTCTCTGCTGGTTACCGTGGCCTTCATCATCTTTTGGGTCCGAAGCA |
| | | AGCGGAGCCGGCTGCTGCACTCCGACTACATGAACATGACCCCTAGACGG |
| | | CCCGGACCAACCAGAAAGCACTACCAGCCTTACGCTCCTCCTAGAGACTT |
| | | CGCCGCCTACCGGTCCAGAGTGAAGTTCAGCAGATCCGCCGATGCTCCCG |
| | | CCTATCAGCAGGGCCAAAACCAGCTGTACAACGAGCTGAACCTGGGGAG |
| | | AAGAGAAGAGTACGACGTGCTGGACAAGCGGAGAGGCAGAGATCCTGAA |
| | | ATGGGCGGCAAGCCCAGACGGAAGAATCCTCAAGAGGGCCTGTATAATG |
| | | AGCTGCAGAAAGACAAGATGGCCGAGGCCTACAGCGAGATCGGAATGAA |
| | | GGGCGAGCGCAGAAGAGGCAAGGGACACGATGGACTGTACCAGGGCCTG |
| | | AGCACCGCCACCAAGGATACCTATGATGCCCTGCACATGCAGGCCCTGCC |
| | | TCCAAGA |
| 58 | 164 | TCACCCTCTTTTCCGGTGGTCCGGACCCAGACCACCGTTACTCCATTCAGC |
| | | TTCTTCGGAACCTGTTTGGAGGAATTAAACGGGCACCCACCCACCTTCAC |
| | | CCCCTTTTCGTAACTAAGTGTGTGCCCAATCTCATGACTCCTGCTGACTTC |
| | | ACCGACCAGCAGTGTCCAAAACGCTAGGTGAATTTCCTTCCTCCCCCTCTG |
| | | GGCTTCTGCCCAGCTCCCTCCCTCCAGCCTGACGTGCCACAGGCTGTGCA |
| | | AAGACCCCGCGAAAGCTGCCAAAAGTGGCAATTGTGGGTCCCCCCTTTGT |
| | | AAAGGCGTCGAGTCTTTCTCCCTTAAGGCTAGACCCGTCAGTGAATTCTGT |
| | | CGGGCAACTAGTGACGCCACTGCATGCCTCCGACCTCGGCCGCGGAGTGC |
| | | TGCCCCCCAAGTCGTGCCCCTGACTACAAGTTGTGCTGTCTGGCAAACATT |
| | | GTCTGTGAGAATGTTCCGCTGTGGCTGCCAAGCCTGGTAACAGGCTGCCC |
| | | CAGTGTGCGTAGTTCTCATCCAGACTTCGGTCTGGCAACTTGCTGTTAAGA |
| | | CACGGCGTAAGGGGCGTGTGCCAACGCCCTGGAACGAGTGTCCACTCTAA |
| | | TACCCCGAGGAATGCTACGCAGGTACCCCTGGCTCCCCAGGGATCTGAGC |
| | | GTAGGCTAATTGTCTAAGGGTATTTTCATTTCCCACTCTTTCTTTCTTGTTC |
| | | ATAATGGCCCTTCCCGTCACCGCTCTCCTCCTGCCACTGGCCTTGCTGCTG |
| | | CACGCTGCACGGCCAGACATCCAGATGACCCAGACAACCAGCTCTCTGTC |
| | | AGCCTCTCTCGGCGATCGCGTCACAATCAGCTGCCGCGCTTCCCAAGACA |
| | | TCTCCAAGTACCTGAACTGGTACCAGCAAAAGCCCGACGGCACCGTGAAG |
| | | CTGCTCATCTACCACACCTCCAGACTGCATAGCGGGGTGCCCAGCAGATT |
| | | CAGTGGCTCAGGCTCAGGCACCGACTACAGCCTGACCATCTCCAACCTGG |
| | | AGCAGGAGGACATTGCCACATACTTCTGCCAGCAGGGCAACACCCTGCCC |
| | | TACACCTTCGGAGGCGGCACAAAGCTGGAGATCACCGGTGGAGGAGGGA |
| | | GTGGAGGAGGAGGCAGTGGTGGCGGAGGTTCCGAGGTGAAGCTCCAGGA |
| | | ATCAGGTCCAGGACTGGTCGCCCCTTCCCAGTCCCTGTCCGTCACCTGCAC |
| | | CGTGAGTGGCGTCAGCCTCCCAGACTACGGTGTGTCTTGGATCCGCCAAC |
| | | CTCCTCGCAAAGGCCTGGAATGGCTCGGCGTCATCTGGGGAAGCGAGACA |
| | | ACCTACTATAACTCCGCACTGAAGTCCCGCCTCACCATCATCAAGGATAA |
| | | TAGCAAGAGCCAGGTCTTCCTCAAGATGAACTCCCTGCAGACCGACGATA |
| | | CCGCCATCTACTACTGTGCCAAGCACTACTACTACGGAGGTTCTTACGCC |
| | | ATGGATTACTGGGGACAGGGAACCTCTGTCACCGTCAGCTCCATCGAGGT |
| | | CATGTATCCACCACCCTACCTGGACAACGAAAAGAGCAATGGCACCATCA |
| | | TCCACGTGAAGGGGAAGCACCTCTGCCCCTCACCCCTGTTCCCTGGTCCCT |
| | | CCAAGCCTTTCTGGGTCCTGGTCGTCGTGGGAGGCGTGTTGGCCTGTTACT |
| | | CCCTGCTCGTCACCGTCGCCTTCATCATCTTCTGGGTTAGGAGTAAGCGGT |
| | | CCCGGCTTCTGCACTCTGACTACATGAACATGACACCCAGAAGACCTGGG |
| | | CCAACCCGGAAGCACTACCAGCCCTACGCTCCACCCAGGGACTTTGCAGC |
| | | CTACAGGTCCCGCGTCAAGTTCTCCCGGTCTGCTGACGCACCTGCCTACCA |
| | | GCAGGGCCAAAACCAGCTCTACAACGAGTTGAACCTCGGCAGACGGGAG |
| | | GAGTACGACGTCCTCGACAAAAGGCGGGGTCGGGATCCTGAGATGGGCG |
| | | GTAAGCCAAGGCGGAAGAACCCACAGGAAGGCCTCTATAATGAGCTCCA |
| | | GAAGGATAAGATGGCTGAGGCCTACTCCGAGATCGGGATGAAGGGCGAA |
| | | AGGAGACGGGGTAAGGGGCACGACGGCCTCTATCAGGGTCTGAGCACCG |
| | | CCACCAAGGACACCTACGACGCCCTGCACATGCAGGCACTGCCACCTCGG |
| 59 | 171 | TCTGTCCTCACCCCATCTTCCCTTCTTTCCTGCACCGTTACGCTTACTCGCA |
| | | TGTGCATTGAGTGGTGCACGTGCTTGAACAAACAGCTACACTCACATGGG |
| | | GGCGGGTTTTCCCGCCCTGCGGCCTCTCGCGAGGCCCACCCCTCCCCTTCC |
| | | TCCCATAACTACAGTGCTTTGGTAGGTAAGCATCCTGATCCCCCGCGGAA |
| | | GCTGCTCACGTGGCAACTGTGGGGACCCAGACAGGTTATCAAAGGCACCC |
| | | GGTCTTTCCGCCTTCAGGAGTATCCCTGCTAGTGAATTCTAGTAGGGCTCT |
| | | GCTTGGTGCCAACCTCCCCCAAATGCGCGCTGCGGGAGTGCTCTTCCCCA |
| | | ACTCACCCTAGTATCCTCTCATGTGTGTGCTTGGTCAGCATATCTGAGACG |
| | | ATGTTCCGCTGTCCCAGACCAGTCCAGTAATGGACGGGCCAGTGTGCGTA |
| | | GTCGTCTTCCGGCTTGTCCGGCGCATGTTTGGTGAACCGGTGGGGTAAGG |
| | | TTGGTGTGCCCAACGCCCGTACTTTGGTGATACCTCAAGACCACCCAGGA |
| | | ATGCCAGGGAGGTACCCCGCTTCACAGCGGGATCTGACCCTGGGCTAATT |
| | | GTCTACGGTGGTTCTTCTTGCTTCCACTTCTTTCTACTGTTCATGATGGCTC |
| | | TGCCTGTGACAGCTCTGCTGCTGCCTCTGGCTCTGCTTCTGCATGCCGCCA |
| | | GACCTGACATCCAGATGACCCAGACAACCAGCAGCCTGTCTGCCAGCCTG |
| | | GGCGATAGAGTGACCATCAGCTGTAGAGCCAGCCAGGACATCAGCAAGT |
| | | ACCTGAACTGGTATCAGCAGAAACCCGACGGCACCGTGAAGCTGCTGATC |
| | | TACCACACCAGCAGACTGCACAGCGGCGTGCCAAGCAGATTTTCTGGCAG |
| | | CGGCTCTGGCACCGACTACAGCCTGACAATCAGCAACCTGGAACAAGAG |
| | | GATATCGCTACCTACTTCTGCCAGCAAGGCAACACCCTGCCTTACACCTTT |
| | | GGCGGAGGCACCAAGCTGGAAATCACAGGGGGGGGAGGAAGCGGAGGC |
| | | GGAGGATCTGGTGGTGGTGGATCTGAAGTGAAACTGCAAGAGTCTGGCCC |
| | | TGGCCTGGTGGCCCCATCTCAATCTCTGAGCGTGACCTGTACCGTCAGCG |
| | | GAGTGTCCCTGCCTGATTATGGCGTGTCCTGGATCCGGCAGCCTCCTAGA |
| | | AAAGGCCTGGAATGGCTGGGCGTGATCTGGGGCAGCGAGACAACCTACT |
| | | ACAACAGCGCCCTGAAGTCCCGGCTGACCATCATCAAGGACAACTCCAAG |
| | | AGCCAGGTGTTCCTGAAGATGAACAGCCTGCAGACCGACGACACCGCCAT |
| | | CTACTATTGCGCCAAGCACTACTACTACGGCGGCAGCTACGCCATGGATT |
| | | ATTGGGGCCAGGGCACCAGCGTGACCGTGTCTAGCATCGAAGTGATGTAC |
| | | CCTCCACCTTACCTGGACAACGAGAAGTCCAACGGCACCATCATCCACGT |
| | | GAAGGGCAAGCACCTGTGTCCTTCTCCACTGTTCCCCGGACCTAGCAAGC |
| | | CTTTCTGGGTGCTCGTTGTTGTTGGCGGCGTGCTGGCCTGTTACTCTCTGC |
| | | TGGTTACCGTGGCCTTCATCATCTTTTGGGTCCGAAGCAAGCGGAGCCGG |
| | | CTGCTGCACTCCGACTACATGAACATGACCCCTAGACGGCCCGGACCAAC |
| | | CAGAAAGCACTACCAGCCTTACGCTCCTCCTAGAGACTTCGCCGCCTACC |
| | | GGTCCAGAGTGAAGTTCAGCAGATCCGCCGATGCTCCCGCCTATCAGCAG |
| | | GGCCAAAACCAGCTGTACAACGAGCTGAACCTGGGGAGAAGAGAAGAGT |
| | | ACGACGTGCTGGACAAGCGGAGAGGCAGAGATCCTGAAATGGGCGGCAA |
| | | GCCCAGACGGAAGAATCCTCAAGAGGGCCTGTATAATGAGCTGCAGAAA |
| | | GACAAGATGGCCGAGGCCTACAGCGAGATCGGAATGAAGGGCGAGCGCA |
| | | GAAGAGGCAAGGGACACGATGGACTGTACCAGGGCCTGAGCACCGCCAC |
| | | CAAGGATACCTATGATGCCCTGCACATGCAGGCCCTGCCTCCAAGA |
| 60 | 177 | TCTGTCCTCACCCCATCTTCCCTTCTTTCCTGCACCGTTACGCTTACTCGCA |
| | | TGTGCATTGAGTGGTGCACGTGCTTGAACAAACAGCTACACTCACATGGG |
| | | GGCGGGTTTTCCCGCCCTGCGGCCTCTCGCGAGGCCCACCCCTCCCCTTCC |
| | | TCCCATAACTACAGTGCTTTGGTAGGTAAGCATCCTGATCCCCCGCGGAA |
| | | GCTGCTCACGTGGCAACTGTGGGGACCCAGACAGGTTATCAAAGGCACCC |
| | | GGTCTTTCCGCCTTCAGGAGTATCCCTGCTAGTGAATTCTAGTAGGGCTCT |
| | | GCTTGGTGCCAACCTCCCCCAAATGCGCGCTGCGGGAGTGCTCTTCCCCA |
| | | ACTCACCCTAGTATCCTCTCATGTGTGTGCTTGGTCAGCATATCTGAGACG |
| | | ATGTTCCGCTGTCCCAGACCAGTCCAGTAATGGACGGGCCAGTGTGCGTA |
| | | GTCGTCTTCCGGCTTGTCCGGCGCATGTTTGGTGAACCGGTGGGGTAAGG |
| | | TTGGTGTGCCCAACGCCCGTACTTTGGTGATACCTCAAGACCACCCAGGA |
| | | ATGCCAGGGAGGTACCCCGCTTCACAGCGGGATCTGACCCTGGGCTAATT |
| | | GTCTACGGTGGTTCTTCTTGCTTCCACTTCTTTCTACTGTTCATGATGGCAC |
| | | TGCCCGTCACCGCACTCCTGCTCCCACTGGCACTGCTGCTCCATGCAGCTC |
| | | GCCCCGATATCCAGATGACCCAGACCACCTCTAGCCTCAGCGCCTCTCTG |
| | | GGTGACCGCGTCACCATCTCTTGCCGGGCCAGCCAAGACATCTCTAAGTA |
| | | CCTGAACTGGTACCAGCAGAAACCTGACGGAACCGTGAAGCTGCTGATCT |
| | | ACCACACCAGTCGGCTGCATTCCGGGGTGCCTTCCAGGTTCAGCGGTTCC |
| | | GGCTCTGGGACCGATTATAGTCTCACCATCTCCAACCTCGAGCAGGAGGA |
| | | CATCGCAACCTACTTCTGCCAGCAGGGGAACACCCTGCCCTACACCTTCG |
| | | GTGGCGGGACCAAGCTGGAGATCACTGGAGGTGGTGGCAGCGGAGGTGG |
| | | AGGATCAGGTGGAGGCGGTAGCGAGGTGAAGCTGCAGGAGTCCGGACCT |
| | | GGTCTGGTGGCCCCAAGCCAGTCCCTCAGCGTCACCTGCACAGTGTCCGG |
| | | GGTGTCCCTGCCTGACTACGGTGTCTCCTGGATCAGGCAACCACCCCGGA |
| | | AGGGTCTCGAGTGGCTGGGCGTCATCTGGGGCTCCGAGACCACCTACTAC |
| | | AACAGCGCTCTGAAGTCCCGGCTGACCATCATCAAAGACAACTCCAAGAG |
| | | CCAGGTGTTCTTGAAGATGAACTCCCTGCAAACCGATGACACCGCCATCT |
| | | ACTACTGCGCCAAGCACTACTACTATGGCGGTAGCTACGCCATGGATTAT |
| | | TGGGGTCAGGGCACCAGTGTCACCGTCTCCTCCATCGAGGTGATGTACCC |
| | | TCCACCCTATCTGGACAACGAGAAGTCCAACGGCACCATCATCCACGTGA |
| | | AGGGCAAGCACCTGTGCCCTAGCCCTCTGTTCCCAGGACCCTCCAAGCCC |
| | | TTCTGGGTGCTGGTCGTGGTGGGAGGAGTCCTGGCCTGCTATTCCCTCCTC |
| | | GTCACCGTGGCATTTATCATCTTCTGGGTCCGGAGCAAGCGGTCACGCCT |
| | | GCTCCACTCCGACTACATGAACATGACTCCTCGCAGACCTGGACCCACCC |
| | | GGAAGCACTACCAGCCTTATGCCCCACCCCGCGACTTTGCCGCTTACCGC |
| | | TCTCGGGTCAAGTTCTCTCGGTCAGCAGACGCCCCTGCATACCAGCAGGG |
| | | CCAGAACCAGCTGTATAACGAGCTGAACCTCGGCAGACGGGAGGAGTAC |
| | | GATGTGCTGGACAAGAGGAGAGGCAGAGACCCCGAGATGGGTGGTAAGC |
| | | CACGGCGCAAGAACCCACAGGAGGGCTTGTACAACGAACTGCAGAAGGA |
| | | CAAGATGGCCGAGGCCTACAGCGAGATCGGCATGAAGGGAGAGAGGCGC |
| | | AGGGGCAAGGGTCACGACGGCCTGTACCAAGGGCTGTCCACCGCAACCA |
| | | AGGACACCTACGATGCCCTGCACATGCAGGCCCTCCCACCAAGG |
| 61 | 180 | TCTGTCCTCACCCCATCTTCCCTTCTTTCCTGCACCGTTACGCTTACTCGCA |
| | | TGTGCATTGAGTGGTGCACGTGCTTGAACAAACAGCTACACTCACATGGG |
| | | GGCGGGTTTTCCCGCCCTGCGGCCTCTCGCGAGGCCCACCCCTCCCCTTCC |
| | | TCCCATAACTACAGTGCTTTGGTAGGTAAGCATCCTGATCCCCCGCGGAA |
| | | GCTGCTCACGTGGCAACTGTGGGGACCCAGACAGGTTATCAAAGGCACCC |
| | | GGTCTTTCCGCCTTCAGGAGTATCCCTGCTAGTGAATTCTAGTAGGGCTCT |
| | | GCTTGGTGCCAACCTCCCCCAAATGCGCGCTGCGGGAGTGCTCTTCCCCA |
| | | ACTCACCCTAGTATCCTCTCATGTGTGTGCTTGGTCAGCATATCTGAGACG |
| | | ATGTTCCGCTGTCCCAGACCAGTCCAGTAATGGACGGGCCAGTGTGCGTA |
| | | GTCGTCTTCCGGCTTGTCCGGCGCATGTTTGGTGAACCGGTGGGGTAAGG |
| | | TTGGTGTGCCCAACGCCCGTACTTTGGTGATACCTCAAGACCACCCAGGA |
| | | ATGCCAGGGAGGTACCCCGCTTCACAGCGGGATCTGACCCTGGGCTAATT |
| | | GTCTACGGTGGTTCTTCTTGCTTCCACTTCTTTCTACTGTTCATGATGGCTC |
| | | TGCCAGTGACCGCACTGCTGCTGCCCTTAGCCTTACTCCTTCACGCAGCCA |
| | | GGCCCGACATCCAGATGACCCAGACCACCAGCTCCCTTTCCGCAAGCCTC |
| | | GGCGACAGGGTCACCATCTCCTGTCGGGCCAGCCAGGACATCAGCAAGTA |
| | | CCTGAACTGGTACCAGCAGAAGCCCGACGGCACCGTGAAGCTGCTGATCT |
| | | ACCACACCTCACGGCTGCACTCAGGCGTGCCCTCACGGTTTAGCGGATCA |
| | | GGCAGCGGCACCGACTACAGCCTGACTATCAGCAACCTGGAGCAGGAGG |
| | | ACATCGCCACCTACTTCTGCCAGCAGGGCAACACCCTGCCCTACACCTTC |
| | | GGAGGCGGCACCAAGCTGGAGATCACCGGTGGCGGTGGTTCAGGTGGCG |
| | | GAGGCTCAGGAGGAGGCGGCAGCGAGGTGAAGCTGCAGGAGTCAGGTCC |
| | | AGGACTGGTGGCACCCAGCCAGAGCCTGAGCGTGACTTGCACCGTGTCAG |
| | | GCGTGAGCCTGCCAGACTACGGCGTGAGCTGGATCCGGCAGCCTCCTCGG |
| | | AAGGGCTTAGAGTGGCTGGGCGTGATCTGGGGCAGCGAGACCACCTACT |
| | | ACAACTCAGCCCTGAAGAGCCGGCTGACCATCATCAAGGACAACAGCAA |
| | | GAGCCAGGTGTTCCTGAAGATGAACAGCCTGCAGACCGACGACACCGCC |
| | | ATCTACTACTGCGCCAAGCACTACTACTACGGCGGCAGCTACGCCATGGA |
| | | CTACTGGGGACAGGGTACCAGCGTGACCGTGAGCAGCATCGAGGTGATG |
| | | TACCCTCCTCCCTACCTGGACAACGAGAAGAGCAACGGCACCATCATCCA |
| | | CGTGAAGGGCAAGCACCTGTGCCCTAGCCCTTTATTCCCCGGCCCCTCAA |
| | | AACCCTTCTGGGTGCTGGTCGTCGTCGGTGGCGTGCTGGCATGCTACAGC |
| | | CTGCTGGTGACCGTGGCCTTCATCATATTCTGGGTCCGGTCAAAGCGGAG |
| | | CCGGTTACTGCACAGCGACTACATGAACATGACTCCACGGCGTCCAGGTC |
| | | CCACTCGGAAGCACTACCAACCCTACGCTCCTCCCCGTGACTTTGCTGCCT |
| | | ACCGTAGCCGGGTGAAGTTCTCCAGGAGCGCCGATGCCCCAGCCTACCAG |
| | | CAGGGCCAGAACCAGCTCTACAATGAGCTTAACCTTGGCAGGCGGGAGG |
| | | AGTACGACGTGCTGGACAAGAGGAGGGGCCGTGATCCCGAGATGGGAGG |
| | | CAAGCCCCGTAGGAAGAATCCCCAGGAGGGCCTTTACAACGAGCTCCAG |
| | | AAGGACAAGATGGCCGAGGCCTACAGCGAGATCGGCATGAAGGGAGAGC |
| | | GTAGGCGTGGAAAGGGCCACGACGGCCTGTACCAGGGCCTGAGCACTGC |
| | | TACCAAGGACACCTACGACGCCCTGCACATGCAGGCTCTTCCACCCCGG |

Additional exemplary circular RNA constructs were designed that comprise an IRES of Table 1A and an anti-CD19 binder of Table 2B are set forth in Table 7A below or sequences from the constructs of Table 1B. Certain of the circular RNA constructs in Table 7A include a miR-122 site.

TABLE 7A
Other CD19 constructs (anti-CD19 28-ζ)

| IRES | Codon NT | |
|---|---|---|
| SEQ ID NO: | SEQ ID NO: | |
| 8 | 24 | |
| 8 | 24 | Includes miR-122 site: |
| | | SEQ ID NO: 200 |
| 8 | 25 | |
| 8 | 25 | Includes miR-122 site: |
| | | SEQ ID NO: 200 |
| 8 | 26 | |
| 8 | 27 | |
| 8 | 26 | Includes miR-122 site: |
| | | SEQ ID NO: 200 |
| 8 | 27 | |
| 8 | 28 | Includes miR-122 site: |
| | | SEQ ID NO: 200 |
| 8 | 19 | Includes miR-122 site: |
| | | SEQ ID NO: 200 |
| 4 | 24 | |
| 4 | 24 | Includes miR-122 site: |
| | | SEQ ID NO: 200 |
| 4 | 25 | |
| 4 | 25 | |

Table 7B describes circular RNA constructs that were designed comprising an expression sequence directed to mouse CD19 and an IRES of Table 1A. Certain of the circular RNA constructs in Table 7B include a miR-122 site.

TABLE 7B
Mouse CD19 constructs

| IRES/CO | IRES | Codon NT | |
|---|---|---|---|
| Clone # | SEQ ID NO: | SEQ ID NO: | |
| mCD19-1 | 16 | 100 | |
| mCD19-2 | 8 | 100 | |
| mCD19-3 | 8 | 100 | Includes miR-122 site: |
| | | | SEQ ID NO: 200 |
| mCD19-4 | 18 | 100 | |

### Example 4: Assessment of IRES-CO Constructs

### A. Effect of IRES Sequence on Expression

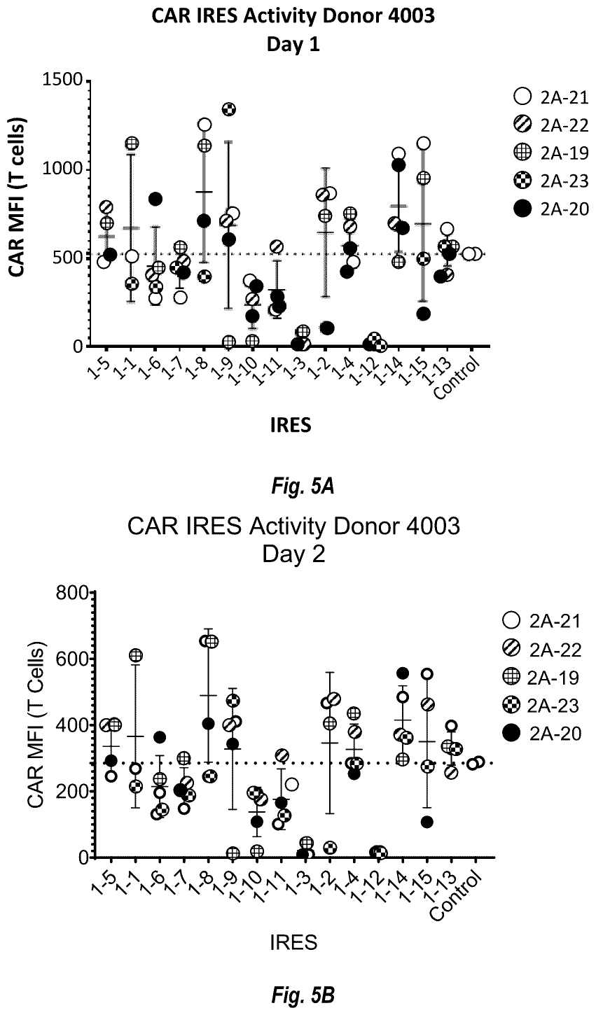

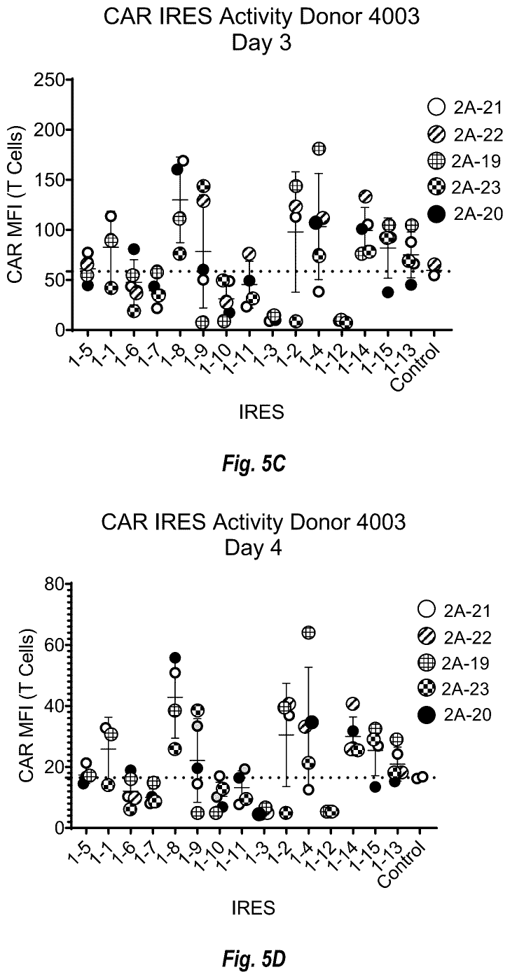

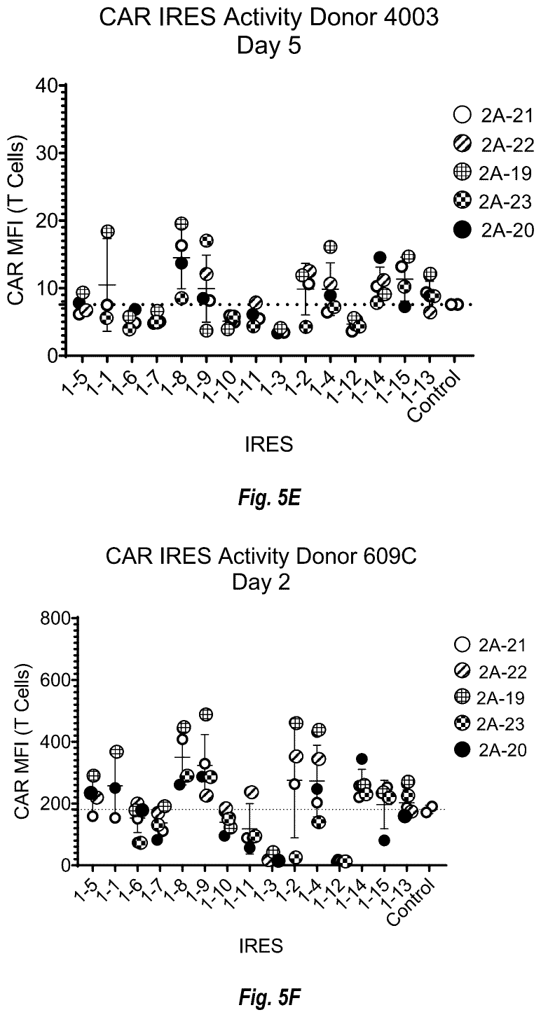

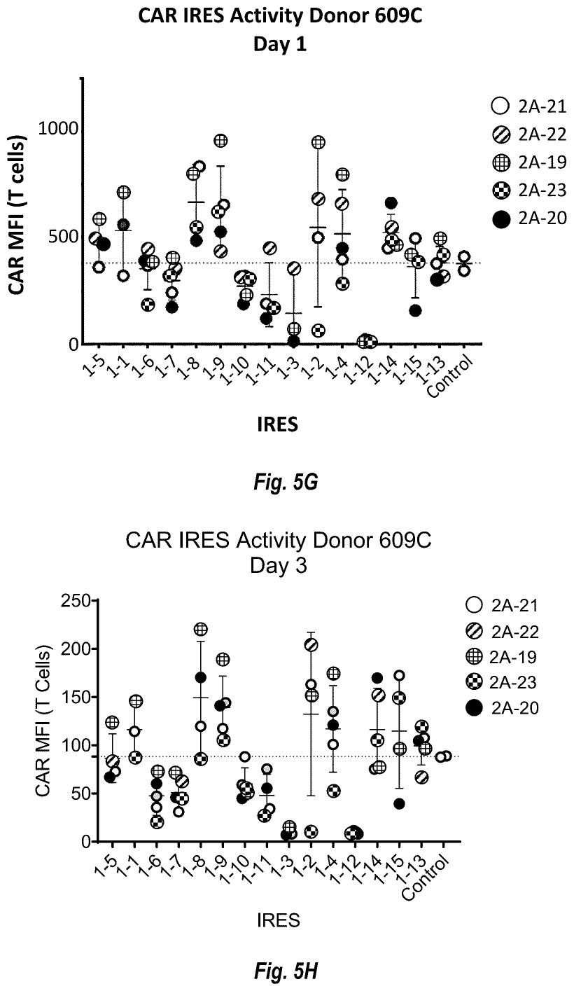

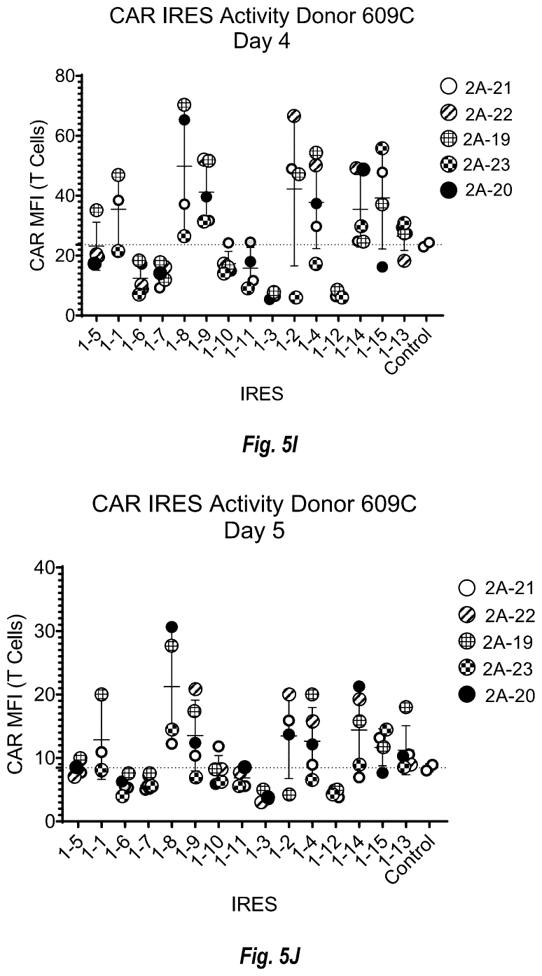

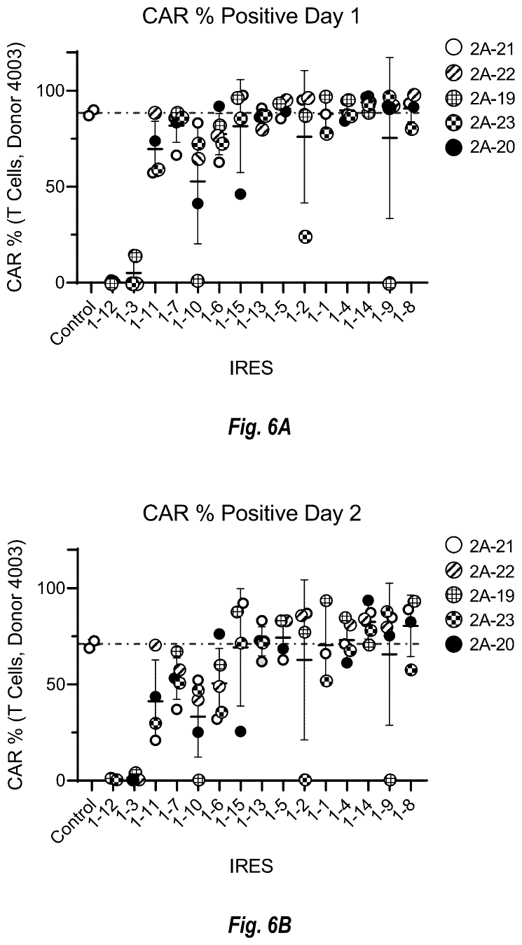

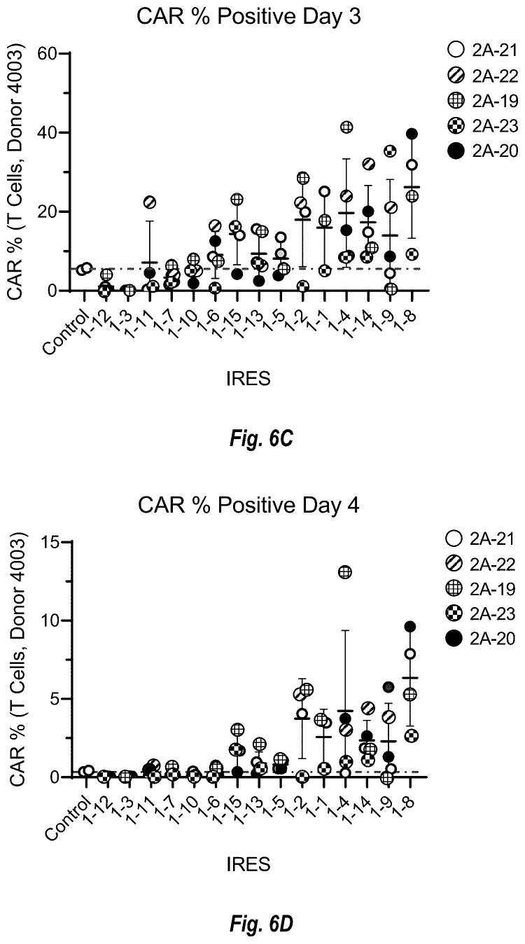

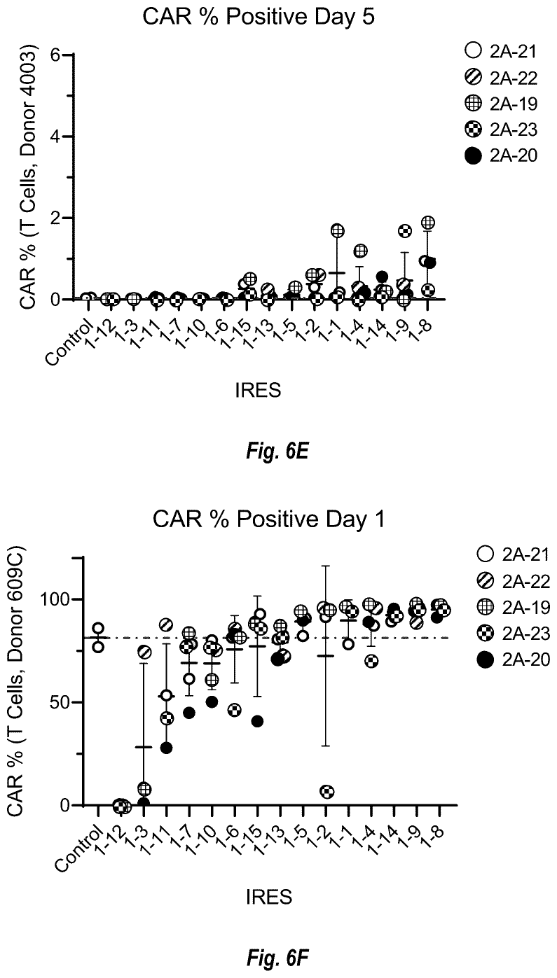

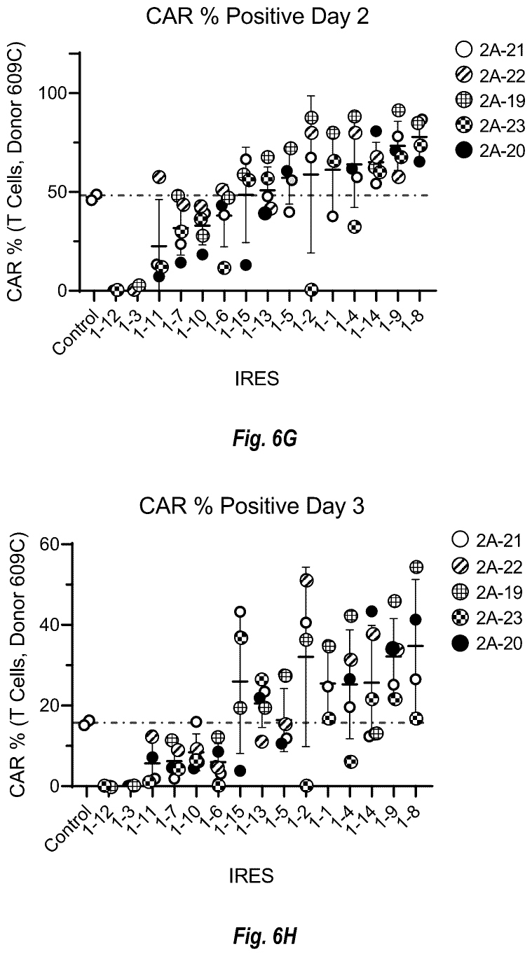

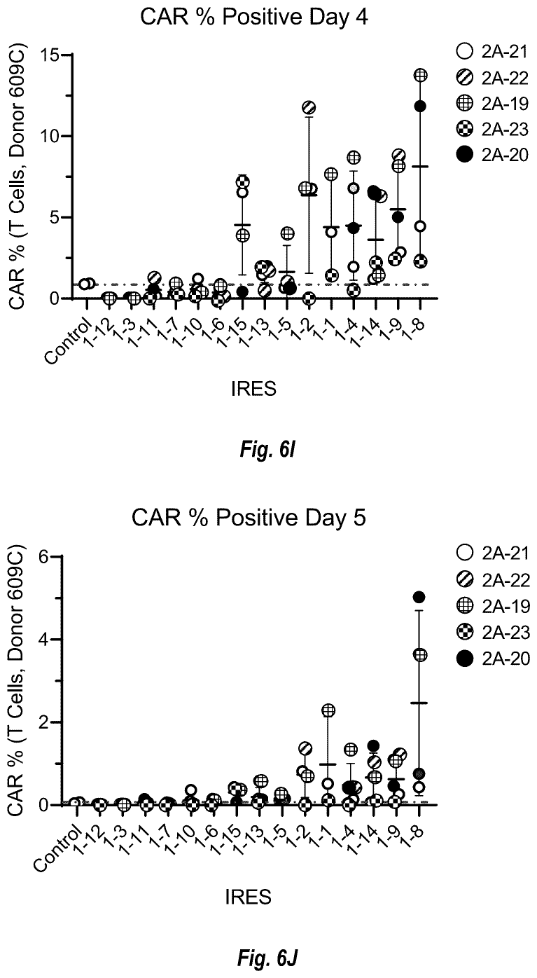

CD3+ T cells from two healthy donors (609C, first row; 4003, second row) were activated with aCD3/CD28 tetrameric complexes for 3 days and then electroporated with CD19 CAR ORNA sequences at a 10 ng/100K cells oRNA dose and evaluated. 74 total sequences were evaluated, comprising a combination of 5 CD19 CAR sequences comprising SEQ ID NOs: 19-23 (the 5 codon optimized sequences of Table 2A) and 16 IRES sequences (including IRESes comprising SEQ ID NOs: 1-15 of Table 1A, and an IRES corresponding to a base CD19 control). Starting at 24 hours post-electroporation up to 120 hours, CD19 CAR expression (T cell MFI), i.e., the level of expression per cell over time, was evaluated via flow cytometry using the anti-idiotypic antibody FMC63. See FIGS. 5A-5J. Starting at 24 hours post-electroporation and up to 120 hours, frequency of CD19 CAR expressing cells (% CAR positive cells by IRES over time, i.e., the percent of cells or average signal of the cells expressing over time) was evaluated via flow cytometry using the anti-idiotypic antibody FMC63. See FIGS. 6A-6J.

B. Effect of IRES Sequence on oCAR™ Stability

CD3+ T cells from two healthy donors (4003, first row; 609C, second row) were activated with aCD3/CD28 tetrameric complexes for 3 days and then electroporated with CD19 CAR ORNA sequences. 74 total sequences were evaluated, comprising a combination of 5 CD19 CAR sequences comprising SEQ ID NOs: 19-23 (the 5 codon optimized sequences of Table 2A) and 18 IRES sequences (including IRESes comprising SEQ ID NOs: 1-15 of Table 1A) (see, e.g., Table 1B). Frequency of CD19 CAR expressing cells was analyzed via flow cytometry at 24-120 hr timepoints post-electroporation. Frequency of CD19 CAR expression was rank ordered for each timepoint pursuant to the tables below for each of Donor 609C and Donor 4003 as compared to a base control CD19 CAR and a combination HER2 standard control sequence.

TABLE
Donor 609C oCAR + cells (% T cells post- electroporation)

| Day 1 | | | Day 2 | | | Day 3 | | | Day 4 | | | Day 5 | | |
|---|---|---|---|---|---|---|---|---|---|---|---|---|---|---|
| 97 | | | 97 | | | 87 | | | 87 | | | 86 | | |
| 97.9 | 97.5 | 97.8 | 91.1 | 90.8 | 92.3 | 53.3 | 53.7 | 56.2 | 13.1 | 13.6 | 14.8 | 3.28 | 4.74 | 7.06* |
| 81 | | | 37 | | | 14 | | | 86 | | | 87 | | |
| 97.1 | 97.5 | 97.4 | 86.9 | 89.8 | 88.5 | 48.6 | 52.7 | 52.8 | 9.96 | 13.2 | 12.4 | 3.11 | 4.07 | 3.77 |
| 87 | | | 17 | | | 97 | | | 14 | | | 7 | | |
| 97 | 97.4 | 97.5 | 85.8 | 88.7 | 88.1 | 43.9 | 45.6 | 48.3 | 9.55 | 12.5 | 13.3 | 2.03 | 1.74 | 3.12 |
| 37 | | | 81 | | | 166 | | | 94 | | | 166 | | |
| 97.3 | 97.2 | 97.1 | 83.9 | 89.1 | 87.4 | 44.4 | 41.7 | 44 | 4.27 | 17.1* | 5.14 | 1.01 | 1.52 | 1.77 |
| 7 | | | 87 | | | 171 | | | 37 | | | 14 | | |
| 97 | 96.5 | 71.4* | 84.7 | 86.7 | 85 | 41 | 44.1 | 44.7 | 8.35 | 8.54 | 9.41 | 1.54 | 1.17 | 1.46 |
| 14 | | | 166 | | | 37 | | | 97 | | | 37 | | |
| 96.7 | 96.1 | 95.9 | 79.1 | 80.7 | 82.4 | 41.7 | 44.6 | 40.9 | 7.41 | 7.86 | 9.42 | 1.1 | 1.83 | 1.16 |
| 91 | | | 7 | | | 86 | | | 7 | | | 94 | | |
| 96.8 | 95.8 | 95.7 | 79.8 | 78.4 | 83.5 | 41.6 | 41.6 | 40.8 | 7.4 | 7.8 | 7.92 | 0.71 | 0.96 | 2.03 |
| 34 | | | 14 | | | 11 | | | 180 | | | 97 | | |
| 96.1 | 95.6 | 95.2 | 75.5 | 82 | 82.4 | 43.5 | 39.5 | 38.7 | 6.65 | 7.44 | 7.58 | 0.93 | 0.83 | 1.53 |
| 166 | | | 34 | | | 164 | | | 17 | | | 164 | | |
| 95.4 | 95.4 | 95.4 | 78.7 | 79.9 | 80.1 | 37.9 | 36.1 | 39 | 5.91 | 6.38 | 8.26 | 0.96 | 1.17 | 1 |
| 17 | | | 91 | | | 180 | | | 34 | | | 11 | | |
| 95.6 | 95.1 | 95.3 | 76.6 | 78.1 | 79.8 | 37.3 | 36.6 | 37.6 | 4.26 | 11.3* | 4.83 | 0.89 | 0.65 | 0.91 |
| 90 | | | 90 | | | 17 | | | 11 | | | 90 | | |
| 94.6 | 94.6 | 94.4 | 72.5 | 75.3 | 73 | 37.8 | 35.8 | 35.2 | 6.45 | 7.04 | 6.83 | 0.71 | 0.76 | 0.8 |
| 100 | | | 57 | | | 7 | | | 166 | | | 17 | | |
| 94.6 | 94.4 | 94.5 | 69.5 | 73.3 | 73 | 35.6 | 35 | 34.5 | 6.77 | 6.11 | 6.96 | 0.75 | 0.62 | 0.71 |
| 96 | | | 96 | | | 94 | | | 171 | | | 167 | | |
| 94.9 | 93.6 | 94.5 | 70.1 | 70.8 | 72 | 31.6 | 34.7 | 35.9 | 4.79 | 6.72 | 8.12 | 0.55 | 0.9 | 0.6 |
| 57 | | | 164 | | | 96 | | | 164 | | | 137 | | |
| 95 | 93.9 | 93.9 | 67.2 | 69 | 68.6 | 31.7 | 35.8 | 34.4 | 6.15 | 6.77 | 6.2 | 0.084 | 0.35 | 1.33 |
| 10 | | | 137 | | | 34 | | | 96 | | | 1 | | |
| 95 | 94.2 | 93.4 | 68.5 | 68.6 | 67.1 | 32.5 | 31.2 | 30.8 | 4.92 | 4.8 | 5.33 | 0.33 | 0.4 | 0.82 |
| 164 | | | 100 | | | 57 | | | 81 | | | 96 | | |
| 94.3 | 93.3 | 93.6 | 68.5 | 69.7 | 65.7 | 27.8 | 28 | 27.4 | 4.17 | 4.62 | 4.57 | 0.42 | 0.41 | 0.56 |
| 171 | | | 11 | | | 36 | | | 36 | | | 34 | | |
| 93.3 | 92.2 | 93 | 64 | 71.2 | 67.1 | 26.4 | 26.5 | 26.8 | 5.6 | 3.47 | 3.97 | 0.51 | 0.45 | 0.39 |
| 170 | | | 171 | | | 140 | | | 1 | | | 180 | | |
| 93.1 | 91.5 | 77.8* | 67.9 | 67 | 64.8 | 25.7 | 24.7 | 29.2 | 4.02 | 3.65 | 4.61 | 0.3 | 0.55 | 0.45 |
| 11 | | | 10 | | | 81 | | | 57 | | | 81 | | |
| 92.1 | 91.9 | 90.4 | 63.9 | 65.3 | 67.2 | 24.3 | 28.6 | 26.6 | 4.36 | 3.92 | 3.92 | 0.34 | 0.46 | 0.49 |
| 167 | | | 86 | | | 91 | | | 177 | | | 36 | | |
| 91.9 | 90.8 | 91.1 | 57.3* | 66 | 64.7 | 25 | 24.6 | 25.9 | 3.88 | 2.51 | 5.45 | 0.47 | 0.42 | 0.38 |
| 86 | | | 167 | | | 1 | | | 91 | | | 177 | | |
| 92 | 90.4 | 91.2 | 61.3 | 62.5 | 62.5 | 24.3 | 26.9 | 23 | 2.66 | 2.95 | 2.94 | 0.32 | 0.47 | 0.31 |
| 54 | | | 36 | | | 131 | | | 100 | | | 171 | | |
| 91.5 | 90.2 | 100* | 59.7 | 63.2 | 62.5 | 23 | 22.5 | 24.9 | 2.4 | 2.93 | 2.27 | 0.28 | 0.36 | 0.45 |
| 56 | | | 56 | | | 136 | | | 90 | | | 101 | | |
| 89.7 | 89.9 | 89.8 | 60.4 | 60 | 61.4 | 21.8 | 21.1 | 22.9 | 2.45 | 2.39 | 2.36 | 0.18 | 0.38 | 0.52 |
| 94 | | | 170 | | | 170 | | | 170 | | | 57 | | |
| 91 | 89.2 | 88.5 | 61.3 | 58.9 | 60.1 | 21.7 | 20.5 | 23 | 2.17 | 2.38 | 2.32 | 0.19 | 0.2 | 0.46 |
| 161 | | | 177 | | | 100 | | | HER2 std_2 | | | 91 | | |
| 90.1 | 88.3 | 89.2 | 57 | 60.1 | 58.9 | 21.9 | 21.8 | 21.5 | 0.03 | 6.42 | 0.054 | 0.11 | 0.25 | 0.42 |
| 36 | | | 94 | | | 137 | | | 136 | | | 51 | | |
| 90.8 | 88.4 | 87.9 | 56.3 | 59 | 59.1 | 21.2 | 17.9 | 19.9 | 2.33 | 2.11 | 1.57 | 0.12 | 0.13 | 0.21 |
| 177 | | | 114 | | | 31 | | | 140 | | | 131 | | |
| 88.9 | 90.2 | 87.9 | 56.5 | 58.6 | 58.2 | 21.5 | 20.2 | 17.2 | 1.13 | 2.15 | 2.72 | 0.085 | 0.15 | 0.22 |
| 114 | | | 31 | | | 177 | | | 31 | | | 67 | | |
| 88.4 | 87.5 | 87.5 | 56.3 | 55.4 | 60.6 | 19.7 | 19.9 | 19.2 | 1.89 | 1.8 | 2.19 | 0.11 | 0.15 | 0.18 |
| 137 | | | 140 | | | 90 | | | 137 | | | 116 | | |
| 88.1 | 86.9 | 87.9 | 55.3 | 58.2 | 57.9 | 16.2 | 16.5 | 17.8 | 1.59 | 1.66 | 1.98 | 0.065 | 0.13 | 0.24 |
| 31 | | | 54 | | | 10 | | | 167 | | | 10 | | |
| 88.4 | 87 | 86.2 | 55.2 | 62.5 | 50.2 | 16.4 | 18.1 | 15.3 | 1.55 | 1.58 | 1.73 | 0.15 | 0.11 | 0.16 |
| 180 | | | 180 | | | CD19 base_1 | | | 131 | | | 31 | | |
| 87.6 | 84.6 | 86.9 | 72.2 | 47.4 | 47.7 | 16 | 15.8 | 17.2 | 1.54 | 1.09 | 1.75 | 0.071 | 0.11 | 0.23 |
| CD19 base_1 | | | 161 | | | 101 | | | 10 | | | 136 | | |
| 87 | 85 | 84.6* | 53.1 | 55.5 | 54 | 15.4 | 15.6 | 16.9 | 1.38 | 1.36 | 1.36 | 0.12 | 0.1 | 0.19 |
| 64 | | | 64 | | | 54 | | | 114 | | | 54 | | |
| 86.3 | 86.5 | 84.5 | 49.5 | 51.3 | 51.4 | 16 | 15.2 | 15.2 | 1.11 | 1.51 | 1.28 | 0.11 | 0.038 | 0.19 |
| 77 | | | CD19 base_1 | | | CD19 base_2 | | | 101 | | | 170 | | |
| 85.5 | 82.3 | 83 | 50.7 | 46.7 | 49 | 14.8 | 16.1 | 14.5 | 1.16 | 1.22 | 1.29 | 0.076 | 0.075 | 0.17 |
| 66 | | | 131 | | | 167 | | | 161 | | | 56 | | |
| 84.4 | 82.7 | 83.3 | 48.7 | 46.2 | 48.3 | 13.4 | 12 | 14.1 | 1.15 | 1.38 | 1.06 | 0.098 | 0.13 | 0.079 |
| 140 | | | 77 | | | 114 | | | 54 | | | 104 | | |
| 83.6 | 82.2 | 82.2 | 46.8 | 48.8 | 47 | 11.2 | 13.4 | 13.3 | 1.1 | 1.09 | 1.01 | 0.064 | 0.083 | 0.13 |
| 51 | | | 67 | | | 67 | | | 77 | | | 100 | | |
| 83.6 | 80.8 | 0* | 31.3* | 45.9 | 47.4 | 12.6 | 11.7 | 12.9 | 1.08 | 1.04 | 0.81 | 0.062 | 0.065 | 0.12 |
| 67 | | | CD19 base_2 | | | 161 | | | CD19 base_1 | | | 176 | | |
| 83.9 | 81.1 | 79.9 | 46.8 | 46.1 | 44.6 | 12.7 | 11.6 | 12.8 | 0.9 | 0.89 | 1 | 0.071 | 0.065 | 0.088 |
| 61 | | | 66 | | | 51 | | | 67 | | | CD19 base_1 | | |
| 83.2 | 30.8* | 79.6 | 41.4* | 47.9 | 38.5 | 11.9 | 11.4 | 12.3 | 0.74 | 0.79 | 1.14 | 0.093 | 0.024 | 0.08 |
| 131 | | | 74 | | | 77 | | | CD19 base_2 | | | 161 | | |
| 82.2 | 79.8 | 80 | 39.9 | 46.7 | 41.9 | 10.5 | 10.5 | 12.6 | 0.64 | 0.93 | 1.06 | 0.048 | 0.075 | 0.074 |
| 101 | | | 134 | | | 134 | | | 56 | | | 134 | | |
| 82.6 | 79.2 | 78.7 | 48.6 | 38.5 | 38.6 | 10.4 | 10.9 | 11.4 | 0.65 | 0.87 | 0.89 | 0.072 | 0.082 | 0.037 |
| 1 | | | 104 | | | 56 | | | 51 | | | 77 | | |
| 80.4 | 77.9 | 76.3 | 40.1 | 41.9 | 43.6 | 10.6 | 10.1 | 11.1 | 0.69 | 0.48 | 0.81 | 0.096 | 0.067 | 0.023 |
| 74 | | | 51 | | | 104 | | | 66 | | | 114 | | |
| 79.7 | 77.2 | 77.7 | 29.2* | 40.6 | 39.2 | 8.91 | 8.79 | 10.3 | 0.59 | 0.63 | 0.66 | 0.062 | 0.051 | 0.054 |
| 80 | | | 136 | | | 74 | | | 104 | | | 40 | | |
| 79.1 | 77.5 | 76.5 | 38.3 | 41.4 | 39.1 | 8.17 | 8.22 | 11 | 0.6 | 0.49 | 0.76 | 0.049 | 0.048 | 0.048 |
| CD19 base_2 | | | 101 | | | 66 | | | 134 | | | 76 | | |
| 77.9 | 75.7 | 76.9 | 27.6* | 40.4 | 38.2 | 7.68 | 9.72 | 8.4 | 0.54 | 0.59 | 0.7 | 0.032 | 0.043 | 0.069 |
| 110 | | | 61 | | | 116 | | | 40 | | | HER2 std_2 | | |
| 77.3 | 76.8 | 75.8 | 37.7 | 39.3 | 38 | 7.06 | 6.8 | 7.64 | 0.63 | 0.64 | 0.54 | 0.00737 | 0.055 | 0.078 |
| 104 | | | 1 | | | 110 | | | 116 | | | CD19 base_2 | | |
| 77.3 | 75.4 | 48.4* | 24.4* | 36.2 | 39.1 | 7 | 6.32 | 5.8 | 0.48 | 0.65 | 0.6 | 0.029 | 0.037 | 0.063 |
| 24 | | | 110 | | | 40 | | | 107 | | | 20 | | |
| 0.2* | 75 | 100* | 36.6 | 34.7 | 38.8 | 6.14 | 6.1 | 6.58 | 0.46 | 0.39 | 0.5 | 0.051 | 0.021 | 0.051 |
| 134 | | | 40 | | | 107 | | | 176 | | | 74 | | |
| 75 | 76.6 | 68.2 | 30.9* | 35 | 29.6 | 5.82 | 5.96 | 6.21 | 0.49 | 0.35 | 0.43 | 0.042 | 0.042 | 0.037 |
| 136 | | | 80 | | | 64 | | | 74 | | | 140 | | |
| 72.5 | 71 | 71 | 28.3 | 30.8 | 32.2 | 5.21 | 4.55 | 5.36 | 0.41 | 0.34 | 0.45 | 0.037 | 0.046 | 0.035 |
| 40 | | | 107 | | | 76 | | | 106 | | | 64 | | |
| 69.4 | 70.1 | 71.8 | 26 | 28.9 | 30.2 | 3.97 | 4.97 | 4.8 | 0.3 | 0.37 | 0.33 | 0.047 | 0.035 | 0.036 |
| 71 | | | 71 | | | 80 | | | 76 | | | 66 | | |
| 63.8 | 60 | 60.5 | 23 | 25.3 | 22.8 | 4.17 | 4.91 | 4.25 | 0.31 | 0.46 | 0.2 | 0.041 | 0.046 | 0.031 |
| 107 | | | 106 | | | 106 | | | 110 | | | 106 | | |
| 63.8 | 59.7 | 60.2 | 16.3 | 18.9 | 19.7 | 4.24 | 4.04 | 5.03 | 0.42 | 0.16 | 0.26 | 0.033 | 0.024 | 0.052 |
| 111 | | | 76 | | | 176 | | | 80 | | | 80 | | |
| 55.5 | 52.7 | 52 | 13.9 | 14 | 14.8 | 3.79 | 3.9 | 3.81 | 0.26 | 0.25 | 0.2 | 0.031 | 0.04 | 0.025 |
| 106 | | | 111 | | | 61 | | | 64 | | | Mock | | |
| 52.3 | 48.7 | 49.6 | 12.3 | 13.7 | 14.1 | 2.8 | 3.41 | 3.09 | 0.21 | 0.21 | 0.14 | 0.026 | 0.027 | 0.041 |
| 70 | | | 176 | | | 71 | | | 111 | | | 70 | | |
| 48.2 | 45.2 | 100* | 13.2 | 13.2 | 12.6 | 1.64 | 1.9 | 2.1 | 0.17 | 0.11 | 0.18 | 0.028 | 0.032 | 0.032 |
| 76 | | | 120 | | | 111 | | | 71 | | | 27 | | |
| 47.8 | 43.2 | 43.8 | 10.1 | 13.7 | 12.2 | 1.68 | 2.27 | 1.61 | 0.13 | 0.12 | 0.11 | 0.023 | 0.042 | 0.026 |
| 120 | | | 70 | | | 120 | | | 61 | | | 124 | | |
| 44 | 41.4 | 0.3* | 10.2 | 10.4 | 12.7 | 0.9 | 1.08 | 1.18 | 0.14 | 0.1 | 0.11 | 0.049 | 0.019 | 0.015 |
| 176 | | | 116 | | | 70 | | | 120 | | | 26 | | |
| 42.9 | 38.3 | 41.3 | 7.32 | 7.19 | 7.23 | 0.56 | 0.72 | 0.73 | 0.097 | 0.086 | 0.094 | 0.03 | 0.022 | 0.031 |
| 116 | | | 27 | | | 24 | | | 127 | | | 120 | | |
| 30.4 | 26.3 | 27.2 | 1.67 | 2.32 | 2.02 | 0.17 | 0.17 | 0.29 | 0.077 | 0.098 | 0.047 | 0.023 | 0.016 | 0.044 |
| 27 | | | 20 | | | 27 | | | 20 | | | 110 | | |
| 10 | 8.34 | 7.66 | 0 | 0.76 | 1.06 | 0.19 | 0.16 | 0.16 | 0.11 | 0.067 | 0.04 | 0.00876 | 0.035 | 0.039 |
| 20 | | | 26 | | | 126 | | | 121 | | | 24 | | |
| 6.93 | 7.06 | 7.31 | 0.47 | 0.37 | 0.9 | 0.16 | 0.2 | 0.055 | 0.065 | 0.089 | 0.039 | 0.021 | 0.026 | 0.034 |
| 26 | | | 127 | | | 121 | | | 27 | | | 127 | | |
| 1.89 | 0.16 | 0* | 0.35 | 0.1 | 0.42 | 0.076 | 0.14 | 0.081 | 0.031 | 0.062 | 0.08 | 0.016 | 0.031 | 0.031 |
| 126 | | | 124 | | | HER2 std_1 | | | 126 | | | 61 | | |
| 0.46 | 0.46 | 0.6 | 0.32 | 0.096 | 0.21 | 0.059 | 0.14 | 0.094 | 0.09 | 0.049 | 0.033 | 0.028 | 0.012 | 0.034 |
| HER2 std_2 | | | 126 | | | 124 | | | 24 | | | HER2 std_1 | | |
| 0.46 | 0.67 | 0.31 | 0.25 | 0.26 | 0.067 | 0.11 | 0.12 | 0.045 | 0 | 0.052 | 0.11 | 0.015 | 0.025 | 0.028 |
| 124 | | | 130 | | | 130 | | | Mock | | | 71 | | |
| 0.4 | 0.39 | 0.44 | 0.028 | 0.28 | 0.086 | 0.042 | 0.099 | 0.12 | 0.05 | 0.047 | 0.065 | 0.02 | 0.022 | 0.025 |
| HER2 std_1 | | | 24 | | | HER2 std_2 | | | 26 | | | 130 | | |
| 0.32 | 0.12 | 0.61 | 0* | 0.13 | 0.13 | 0.03 | 0.13 | 0.082 | 0.043 | 0.055 | 0.063 | 0.02 | 0.024 | 0.02 |
| 127 | | | HER2 std_1 | | | Mock | | | HER2 std_1 | | | 121 | | |
| 0.24 | 0.35 | 0.27 | 0.024 | 0.076 | 0.2 | 0.078 | 0.094 | 0.06 | 0.056 | 0.034 | 0.071 | 0.026 | 0.00905 | 0.023 |
| Mock | | | 121 | | | 20 | | | 124 | | | 126 | | |
| 0.24 | 0.23 | 0.25 | 0.13 | 0 | 0.056 | 0.13 | 0.04 | 0.051 | 0.051 | 0.041 | 0.049 | 0.00939 | 0.01 | 0.018 |
| 130 | | | HER2 std_2 | | | 26 | | | 70 | | | 107 | | |
| 0.33 | 0.2 | 0.17 | 0.087 | 0.038 | 0 | 0.072 | 0.05 | 0.096 | 0.021 | 0.064 | 0.048 | 0.037 | 0 | 0 |
| 121 | | | Mock | | | 127 | | | 130 | | | 111 | | |
| 0.22 | 0.32 | 0.11 | 0 | 0 | 0 | 0 | 0.1 | 0.021 | 0.055 | 0.037 | 0.036 | 0.00878 | 0.027 | 0 |

TABLE
Donor 4003 oCAR + cells (% T cells post- electroporation)

| Day 1 | Day 2 | Day 3 |
|---|---|---|
| 171 | 166 | 37 |

| | | | | | | | | |
|---|---|---|---|---|---|---|---|---|
| 97.8 | 97.5 | 97.6 | 93.7 | 93.9 | 93.5 | 50.9 | 43 | 30.5* |

| | | |
|---|---|---|
| 7 | 7 | 81 |

| | | | | | | | | |
|---|---|---|---|---|---|---|---|---|
| 97.6 | 97.8 | 96.8 | 92.9 | 93.5 | 93.5 | 43.1 | 41.5 | 11* |

| | | |
|---|---|---|
| 161 | 87 | 86 |

| | | | | | | | | |
|---|---|---|---|---|---|---|---|---|
| 97.1 | 97.1 | 97.4 | 93.6 | 92.3 | 93.2 | 43.6 | 38.6 | 36.9 |

| | | |
|---|---|---|
| 87 | 171 | 17 |

| | | | | | | | | |
|---|---|---|---|---|---|---|---|---|
| 97.4 | 97.6 | 96.4 | 91.9 | 93 | 91.8 | 38 | 34.2 | 13.9* |

| | | |
|---|---|---|
| 166 | 81 | 100 |

| | | | | | | | | |
|---|---|---|---|---|---|---|---|---|
| 96.8 | 97.2 | 96.2 | 91.2 | 88.1 | 87.4 | 36 | 30.5 | 39.5 |

| | | |
|---|---|---|
| 100 | 100 | 164 |

| | | | | | | | | |
|---|---|---|---|---|---|---|---|---|
| 96.8 | 96.1 | 97.1 | 87.2 | 88 | 88.9 | 33.8 | 29.2 | 33.2 |

| | | |
|---|---|---|
| 177 | 177 | 94 |

| | | | | | | | | |
|---|---|---|---|---|---|---|---|---|
| 96.6 | 96.8 | 96.6 | 88.7 | 100* | 87.2 | 34.8 | 25.6 | 2.61* |

| | | |
|---|---|---|
| 14 | 161 | 14 |

| | | | | | | | | |
|---|---|---|---|---|---|---|---|---|
| 0* | 96.3 | 96 | 89.3 | 84.2 | 88.6 | 29.4 | 27.6 | 9.76* |

| | | |
|---|---|---|
| 91 | 11 | 1 |

| | | | | | | | | |
|---|---|---|---|---|---|---|---|---|
| 95.5 | 95.3 | 95.5 | 86.9 | 87.1 | 86.7 | 24 | 26.2 | 25.2 |

| | | |
|---|---|---|
| 11 | 14 | 11 |

| | | | | | | | | |
|---|---|---|---|---|---|---|---|---|
| 95.7 | 94.9 | 95.3 | 86.7 | 85.7 | 85.3 | 24.6 | 24.3 | 10.9* |

| | | |
|---|---|---|
| 34 | 91 | 34 |

| | | | | | | | | |
|---|---|---|---|---|---|---|---|---|
| 94.8 | 95.4 | 94.4 | 84.9 | 84.6 | 84.7 | 22.6 | 24.6 | 25.1 |

| | | |
|---|---|---|
| 37 | 37 | 87 |

| | | | | | | | | |
|---|---|---|---|---|---|---|---|---|
| 95.3 | 94.6 | 94.5 | 85.5 | 84.1 | 83.9 | 26 | 22.1 | 23.6 |

| | | |
|---|---|---|
| 54 | 54 | 36 |

| | | | | | | | | |
|---|---|---|---|---|---|---|---|---|
| 94.3 | 94.5 | 94.6 | 83.7 | 83.8 | 82.6 | 23.9 | 22.1 | 2.5e−002* |

| | | |
|---|---|---|
| 164 | 131 | 177 |

| | | | | | | | | |
|---|---|---|---|---|---|---|---|---|
| 94 | 95.5 | 93.3 | 83.5 | 83.1 | 82.9 | 25.7 | 22.6 | 20.6 |

| | | |
|---|---|---|
| 57 | 57 | 137 |

| | | | | | | | | |
|---|---|---|---|---|---|---|---|---|
| 94.4 | 93.7 | 92.8 | 83.5 | 82.8 | 82.2 | 23.4 | 21.4 | 0.11* |

| | | |
|---|---|---|
| 81 | 164 | 161 |

| | | | | | | | | |
|---|---|---|---|---|---|---|---|---|
| 93.9 | 93.2 | 93.4 | 83.2 | 82.6 | 82.6 | 23.5 | 20.9 | 0.12* |

| | | |
|---|---|---|
| 170 | 86 | 166 |

| | | | | | | | | |
|---|---|---|---|---|---|---|---|---|
| 93 | 93.4 | 92.1 | 82.6 | 81.9 | 83 | 23 | 19 | 18.2 |

| | | |
|---|---|---|
| 94 | 34 | 7 |

| | | | | | | | | |
|---|---|---|---|---|---|---|---|---|
| 92.8 | 91.9 | 92.4 | 81.4 | 80.7 | 81.1 | 19.9 | 18 | 14.9 |

| | | |
|---|---|---|
| 96 | 94 | 171 |

| | | | | | | | | |
|---|---|---|---|---|---|---|---|---|
| 91.6 | 91.8 | 93 | 81.7 | 78.8 | 80 | 17.9 | 15.6 | 8.54* |

| | | |
|---|---|---|
| 66 | 170 | 180 |

| | | | | | | | | |
|---|---|---|---|---|---|---|---|---|
| 92.4 | 90.9 | 92.4 | 77.8 | 78 | 78.5 | 16.2 | 13.2 | 19.3 |

| | | |
|---|---|---|
| 86 | 17 | 131 |

| | | | | | | | | |
|---|---|---|---|---|---|---|---|---|
| 91.3 | 91.7 | 91.8 | 78.5 | 77.4 | 76.4 | 16.9 | 16.1 | 13.9 |

| | | |
|---|---|---|
| 131 | 66 | 90 |

| | | | | | | | | |
|---|---|---|---|---|---|---|---|---|
| 91 | 91.3 | 90.6 | 74.2 | 77.5 | 76.9 | 15.5 | 12.1 | 0.5* |

| | | |
|---|---|---|
| CD19 base_1 | 96 | 51 |

| | | | | | | | | |
|---|---|---|---|---|---|---|---|---|
| 91.6 | 91.2 | 87.5 | 74.9 | 77 | 74.2 | 14.6 | 13.8 | 12.1 |

| | | |
|---|---|---|
| 56 | 136 | 167 |

| | | | | | | | | |
|---|---|---|---|---|---|---|---|---|
| 88.7 | 89 | 89.8 | 72.7 | 72.4 | 73.3 | 13.7 | 12.3 | 6.67* |

| | | |
|---|---|---|
| 167 | CD19 base_1 | 114 |

| | | | | | | | | |
|---|---|---|---|---|---|---|---|---|
| 89.6 | 87.7 | 89 | 74 | 71.6 | 72.4 | 13.5 | 12.1 | 42.2* |

| | | |
|---|---|---|
| 114 | 137 | 66 |

| | | | | | | | | |
|---|---|---|---|---|---|---|---|---|
| 88.3 | 88.7 | 88 | 73.3 | 72.6 | 71.9 | 15.3 | 13.2 | 9.24 |

| | | |
|---|---|---|
| 17 | 140 | 40 |

| | | | | | | | | |
|---|---|---|---|---|---|---|---|---|
| 88 | 87.6 | 88.9 | 71.8 | 72 | 70.9 | 14.1 | 11 | 0.31* |

| | | |
|---|---|---|
| 137 | 180 | 170 |

| | | | | | | | | |
|---|---|---|---|---|---|---|---|---|
| 87.7 | 89 | 87.5 | 72.3 | 71.1 | 70.7 | 13.2 | 10.2 | 2.72* |

| | | |
|---|---|---|
| 31 | 31 | 140 |

| | | | | | | | | |
|---|---|---|---|---|---|---|---|---|
| 88.3 | 88.3 | 87.4 | 72.8 | 70.7 | 69.7 | 11.4 | 10.2 | 0.072* |

| | | |
|---|---|---|
| 1 | 167 | 134 |

| | | | | | | | | |
|---|---|---|---|---|---|---|---|---|
| 86.1 | 87.9 | 89.5 | 72 | 72 | 67.6 | 10.3 | 8.59 | 0.62* |

| | | |
|---|---|---|
| 140 | 114 | 77 |

| | | | | | | | | |
|---|---|---|---|---|---|---|---|---|
| 87.2 | 87.5 | 88.2 | 70.1 | 69.3 | 71.3 | 9.34 | 8.03 | 2.29* |

| | | |
|---|---|---|
| 77 | CD19 base_2 | 96 |

| | | | | | | | | |
|---|---|---|---|---|---|---|---|---|
| 87.8 | 85.6 | 88 | 69.3 | 69.2 | 58 | 11.2 | 8.15 | 6.62 |

| | | |
|---|---|---|
| CD19 base_2 | 56 | CD19 base_2 |

| | | | | | | | | |
|---|---|---|---|---|---|---|---|---|
| 86.4 | 87.3 | 87.4 | 70 | 0* | 67.2 | 9.65 | 7.56 | 0.08* |

| | | |
|---|---|---|
| 40 | 40 | 54 |

| | | | | | | | | |
|---|---|---|---|---|---|---|---|---|
| 87.6 | 85.1 | 87.6 | 69.5 | 67.8 | 64.9 | 12* | 8.48 | 7.66 |

| | | |
|---|---|---|
| 80 | 77 | 57 |

| | | | | | | | | |
|---|---|---|---|---|---|---|---|---|
| 86.7 | 86.5 | 86.2 | 67.3 | 66.1 | 67.2 | 8.17 | 6.99 | 2.09* |

| | | |
|---|---|---|
| 136 | 1 | 67 |

| | | | | | | | | |
|---|---|---|---|---|---|---|---|---|
| 86.4 | 87.3 | 84.9 | 66.1 | 66.2 | 65.7 | 8.85 | 7.18 | 6.31 |

| | | |
|---|---|---|
| 180 | 51 | 91 |

| | | | | | | | | |
|---|---|---|---|---|---|---|---|---|
| 84.1 | 87.1 | 86.6 | 64 | 61.8 | 62.5 | 5.79 | 5.81 | 1.73* |

| | | |
|---|---|---|
| 74 | 134 | 56 |

| | | | | | | | | |
|---|---|---|---|---|---|---|---|---|
| 86.5 | 84.4 | 86.6 | 61.6 | 63.1 | 60.8 | 5.73 | 5.73 | 0.19* |

| | | |
|---|---|---|
| 51 | 36 | 10 |

| | | | | | | | | |
|---|---|---|---|---|---|---|---|---|
| 84.8 | 84.6 | 86.8 | 62.9 | 60.5 | 60.5 | 5.14 | 5.34 | 0* |

| | | |
|---|---|---|
| 36 | 67 | CD19 base_1 |

| | | | | | | | | |
|---|---|---|---|---|---|---|---|---|
| 83.8 | 83.8 | 85.2 | 60.6 | 58.5 | 61.4 | 5.83 | 4.6 | 5.17 |

| | | |
|---|---|---|
| 76 | 90 | 101 |

| | | | | | | | | |
|---|---|---|---|---|---|---|---|---|
| 82.6 | 83.5 | 84 | 58 | 57.1 | 57.1 | 5.03 | 4.06 | 4.72 |

| | | |
|---|---|---|
| 101 | 74 | 116 |

| | | | | | | | | |
|---|---|---|---|---|---|---|---|---|
| 82.5 | 83.9 | 83.5 | 59.1 | 57.6 | 54 | 4.81 | 4.43 | 4.28 |

| | | |
|---|---|---|
| 67 | 76 | 110 |

| | | | | | | | | |
|---|---|---|---|---|---|---|---|---|
| 81.8 | 81.2 | 83.4 | 56.1 | 51.4 | 52.2 | 4.09 | 3.55 | 8.14* |

| | | |
|---|---|---|
| 90 | 101 | 61 |

| | | | | | | | | |
|---|---|---|---|---|---|---|---|---|
| 81.1 | 79.5 | 82.2 | 53.4 | 51.3 | 51.9 | 4.21 | 3.25 | 18.4* |

| | | |
|---|---|---|
| 134 | 10 | 136 |

| | | | | | | | | |
|---|---|---|---|---|---|---|---|---|
| 81 | 79.4 | 81.4 | 54 | 50 | 20* | 4.56 | 2.81 | 0.08* |

| | | |
|---|---|---|
| 10 | 80 | 64 |

| | | | | | | | | |
|---|---|---|---|---|---|---|---|---|
| 75.6 | 80.4 | 79.5 | 52.8 | 50.8 | 49.1 | 4.21 | 2.73 | 42* |

| | | |
|---|---|---|
| 64 | 64 | 76 |

| | | | | | | | | |
|---|---|---|---|---|---|---|---|---|
| 76.9 | 76.4 | 77.5 | 51.3 | 49 | 47 | 3.34 | 3.22 | 0.22* |

| | | |
|---|---|---|
| 116 | 110 | 176 |

| | | | | | | | | |
|---|---|---|---|---|---|---|---|---|
| 72.4 | 74.3 | 74.9 | 47.3 | 46.6 | 46.3 | 3.67 | 2.78 | 6.18* |

| | | |
|---|---|---|
| 70 | 116 | 31 |

| | | | | | | | | |
|---|---|---|---|---|---|---|---|---|
| 74.2 | 73.5 | 72.1 | 44.1 | 43.2 | 43.7 | 3.29 | 2.96 | 20.4* |

| | | |
|---|---|---|
| 110 | 104 | 74 |

| | | | | | | | | |
|---|---|---|---|---|---|---|---|---|
| 72.3 | 73 | 73.3 | 43.5 | 42 | 40.3 | 2.95 | 2.42 | 6.53* |

| | | |
|---|---|---|
| 71 | 71 | 80 |

| | | | | | | | | |
|---|---|---|---|---|---|---|---|---|
| 65.1 | 66.3 | 68 | 41.3 | 35.8 | 34.1 | 2.79 | 2.03 | 2.19 |

| | | |
|---|---|---|
| 104 | 70 | 104 |

| | | | | | | | | |
|---|---|---|---|---|---|---|---|---|
| 64.8 | 62.2 | 66.3 | 37.6 | 35.9 | 32.9 | 1.94 | 1.25 | 11.8* |

| | | |
|---|---|---|
| 61 | 61 | 120 |

| | | | | | | | | |
|---|---|---|---|---|---|---|---|---|
| 63.6 | 59.7 | 65.1 | 33.9 | 30.5 | 31.4 | 1.31 | 0.98 | 1.25 |

| | | |
|---|---|---|
| 120 | 120 | 70 |

| | | | | | | | | |
|---|---|---|---|---|---|---|---|---|
| 59.7 | 57.4 | 60.9 | 30.5 | 29.8 | 30.1 | 0.58 | 0.58 | 0.28 |

| | | |
|---|---|---|
| 111 | 176 | 111 |

| | | | | | | | | |
|---|---|---|---|---|---|---|---|---|
| 56.4 | 56.7 | 58.5 | 24.9 | 26.5 | 25.1 | 0.58 | 0.33 | 0.018* |

| | | |
|---|---|---|
| 176 | 106 | 71 |

| | | | | | | | | |
|---|---|---|---|---|---|---|---|---|
| 45.9 | 46.1 | 46.4 | 26.2 | 24.6 | 24.6 | 0.27 | 0.53 | 3.85* |

| | | |
|---|---|---|
| 106 | 111 | 106 |

| | | | | | | | | |
|---|---|---|---|---|---|---|---|---|
| 41.8 | 40.7 | 41.2 | 21.9 | 19.1 | 21.9 | 0.35 | 0.27 | 5.01* |

| | | |
|---|---|---|
| 20 | 27 | HER2 std_2 |

| | | | | | | | | |
|---|---|---|---|---|---|---|---|---|
| 22.41 | 26.2 | 25 | 3.77 | 50* | 3.45 | 0.17 | 0.13 | 18* |

| | | |
|---|---|---|
| 27 | 127 | 107 |

| | | | | | | | | |
|---|---|---|---|---|---|---|---|---|
| 12.1 | 15.5 | 16.3 | 1.75 | 0.66 | 1.21 | 0.17 | 0.084 | 24.3* |

| | | |
|---|---|---|
| 107 | 20 | 24 |

| | | | | | | | | |
|---|---|---|---|---|---|---|---|---|
| 1.25 | 1.73 | 2.1 | 1.03 | 0.63 | 0.45 | 0.15 | 0.067* | 0.1 |

| | | |
|---|---|---|
| 126 | 24 | 26 |

| | | | | | | | | |
|---|---|---|---|---|---|---|---|---|
| 1.48 | 0.84 | 1.93 | 1.16 | 0.21 | 0.066 | 0.14 | 0.022* | 0.1 |

| | | |
|---|---|---|
| 124 | 107 | 130 |

| | | | | | | | | |
|---|---|---|---|---|---|---|---|---|
| 1.06 | 1.14 | 0.58 | 0.47 | 0.37 | 0.34 | 0.11 | 0.13 | 0.048* |

| | | |
|---|---|---|
| 130 | 124 | 127 |

| | | | | | | | | |
|---|---|---|---|---|---|---|---|---|
| 0.8 | 0.59 | 0.047 | 0.28 | 0.33 | 0.32 | 0.15 | 0.088 | 11.8* |

| | | |
|---|---|---|
| 127 | 126 | 27 |

| | | | | | | | | |
|---|---|---|---|---|---|---|---|---|
| 0.26 | 0.4 | 0.63 | 0.24 | 0.37 | 0.22 | 0.059 | 0.14 | 4.33* |

| | | |
|---|---|---|
| 97 | HER2 std_2 | 124 |

| | | | | | | | | |
|---|---|---|---|---|---|---|---|---|
| 0.21 | 0.4 | 0.62 | 0.18 | 0.29 | 0.094 | 0.12 | 0.053 | 2.9* |

| | | |
|---|---|---|
| HER2 std_2 | 121 | 97 |

| | | | | | | | | |
|---|---|---|---|---|---|---|---|---|
| 0.31 | 0.42 | 0.23 | 0.075 | 0.17 | 0.2 | 0.077 | 0.069 | 1.54* |

| | | |
|---|---|---|
| 24 | 26 | 20 |

| | | | | | | | | |
|---|---|---|---|---|---|---|---|---|
| 0.34 | 0.23 | 0.31 | 0.21 | 0.19 | 0.03 | 0.067 | 0.056 | 2.96* |

| | | |
|---|---|---|
| 26 | Mock | 126 |

| | | | | | | | | |
|---|---|---|---|---|---|---|---|---|
| 0.23 | 0.39 | 0.19 | 0.17 | 0.21 | 0.042 | 0.058 | 0.058 | 0.33* |

| | | |
|---|---|---|
| Mock | HER2 std_1 | Mock |

| | | | | | | | | |
|---|---|---|---|---|---|---|---|---|
| 0.19 | 0.082 | 0.066 | 0.081 | 0.19 | 0.14 | 0 | 0.17 | 0 |

| | | |
|---|---|---|
| HER2 std_1 | 97 | 121 |

| | | | | | | | | |
|---|---|---|---|---|---|---|---|---|
| 0.15 | 0.12 | 0.045 | 0.16 | 0.19 | 0.042 | 0.05 | 0.058 | 0.17* |

| | | |
|---|---|---|
| 121 | 130 | HER2 std_1 |

| | | | | | | | | |
|---|---|---|---|---|---|---|---|---|
| 0.14 | 0.08 | 0.074 | 0.13 | 0.12 | 0.098 | 0 | 0.081 | 0.037 |

| Day 4 | | | Day 5 | | |
|---|---|---|---|---|---|
| 37 | | | 100 | | |
| 11.6 | 14.5 | 30.7* | 2.1 | 2.56 | 0.4* |
| 86 | | | 87 | | |
| 10.2 | 9.99 | 8.67 | 2.02 | 1.73 | 1.94 |
| 81 | | | 7 | | |
| 8.76 | 7.62 | 7.26 | 2.02 | 1.64 | 1.52 |
| 17 | | | 37 | | |
| 6.18 | 5.09 | 5.45 | 1.32 | 0.89* | 1.29 |
| 14 | | | 86 | | |
| 6.13 | 4.9 | 4.84 | 1.01 | 0.84 | 0.86 |
| 87 | | | 81 | | |
| 6.31 | 4.82 | 4.71 | 1.09* | 0.86 | 0.89 |
| 164 | | | 17 | | |
| 5.22 | 4.52 | 3.54 | 0.69 | 0.61 | 0.51 |
| 11 | | | 14 | | |
| 4.74 | 3.6 | 3.84 | 0.83 | 0.5 | 0.47 |
| 94 | | | 166 | | |
| 4.18 | 3.88 | 3.42 | 0.74 | 0.49 | 0.46 |
| 36 | | | 177 | | |
| 3.89 | 4.36 | 2.98 | 0.57 | 0.53 | 0.4 |
| 7 | | | 171 | | |
| 4.21 | 3.81 | 3.07 | 0.38 | 0.41 | 0.36 |
| 1 | | | 94 | | |
| 3.96 | 3.29 | 3.2 | 0.41 | 0.33 | 0.33 |
| 100 | | | 57 | | |
| 10.4* | 4.19 | 2.68 | 0.35 | 0.31 | 0.28 |
| 177 | | | 11 | | |
| 3.18 | 3.04 | 3.1 | 0.34 | 0.29 | 0.27 |
| 34 | | | 164 | | |
| 3.76 | 3.01 | 2.5 | 0.3 | 0.26 | 0.15* |
| 166 | | | 34 | | |
| 2.81 | 3 | 2.07 | 0.31 | 0.28 | 0.23 |
| 90 | | | 137 | | |
| 2.5 | 2.04 | 3.25 | 0.26 | 0.18 | 0.21 |
| 137 | | | 167 | | |
| 2.73 | 1.77 | 1.86 | 0.21 | 0.23 | 0.19 |
| 161 | | | 1 | | |
| 1.91 | 1.99 | 1.68 | 0.22 | 0.17 | 0.14 |
| 167 | | | 90 | | |
| 1.73 | 1.66 | 1.84 | 0.38* | 0.16 | 0.19 |
| 180 | | | 91 | | |
| 1.98 | 1.73 | 1.5 | 0.16 | 0.14 | 0.086* |
| 171 | | | 96 | | |
| 1.94 | 1.81 | 1.32 | 0.18 | 0.096* | 0.12 |
| 96 | | | 180 | | |
| 1.58 | 1.25 | 1.1 | 0.19 | 0.12 | 0.12 |
| 40 | | | 161 | | |
| 1.1 | 0.94 | 1.03 | 0.14 | 0.12 | 0.11 |
| 131 | | | 56 | | |
| 1 | 1.09 | 0.83 | 0.029* | 0.1 | 0.098 |
| 51 | | | 131 | | |
| 1.3* | 0.95 | 0.96 | 0.12* | 0.092 | 0.082 |
| 57 | | | 170 | | |
| 1.52* | 0.96 | 0.78 | 0.085 | 0.059 | 0.092 |
| 170 | | | 36 | | |
| 1.6* | 0.88 | 0.82 | 0.28* | 0.072 | 0.084 |
| 66 | | | 136 | | |
| 0.89 | 0.78 | 0.59 | 0.13* | 0.065 | 0.078 |
| 114 | | | 114 | | |
| 0.92 | 0.73 | 0.56 | 0.072 | 0.053 | 0.069 |
| 67 | | | 66 | | |
| 0.84 | 0.52 | 0.49 | 0.057 | 0.038 | 0.061 |
| 77 | | | 77 | | |
| 0.79 | 0.56 | 0.49 | 0.042 | 0.064 | 0.045 |
| 54 | | | 176 | | |
| 0.64 | 0.59 | 0.44 | 0.06 | 0.051 | 0.035 |
| 56 | | | 54 | | |
| 0.38 | 0.61 | 0.68 | 0.058 | 0.015* | 0.037 |
| 140 | | | CD19 base_1 | | |
| 0.72 | 0.62 | 0.32 | 0.045 | 0.07 | 0.027 |
| 91 | | | 10 | | |
| 0.6 | 0.6 | 0.41 | 0.02 | 0.064 | 0.055 |
| 10 | | | 40 | | |
| 0.33 | 0.7 | 0.52 | 0.065 | 0.034 | 0.04 |
| 134 | | | 140 | | |
| 0.65 | 0.42 | 0.46 | 0.051 | 0.024 | 0.061 |
| CD19 base_2 | | | 134 | | |
| 0.54 | 0.4 | 0.34 | 0.044 | 0.058 | 0.028 |
| 116 | | | 31 | | |
| 0.43 | 0.43 | 0.31 | 0.034 | 0.047 | 0.042 |
| 101 | | | 67 | | |
| 0.57 | 0.33 | 0.26 | 0.07 | 0.023 | 0.029 |
| CD19 base_1 | | | 110 | | |
| 0.42 | 0.19 | 0.43 | 0.035 | 0.039 | 0.047 |
| 176 | | | 51 | | |
| 0.38 | 0.36 | 0.3 | 0.033 | 0.031 | 0.047 |
| 31 | | | 76 | | |
| 0.3 | 0.25 | 0.27 | 0.049 | 0.027 | 0.027 |
| 136 | | | 101 | | |
| 0.29 | 0.2 | 0.22 | 0.025 | 0.038 | 0.037 |
| 104 | | | 20 | | |
| 0.16 | 0.16 | 0.17 | 0.035 | 0.039 | 0.025 |
| 71 | | | Mock | | |
| 0.07 | 0.15 | 0.16 | 0.028 | 0.05 | 0.018 |
| 110 | | | 74 | | |
| 0.12 | 0.13 | 0.13 | 0.022 | 0.023 | 0.045 |
| 61 | | | CD19 base_2 | | |
| 0.1 | 0.16 | 0.086 | 0.031 | 0.031 | 0.028 |
| 64 | | | 124 | | |
| 0.13 | 0.11 | 0.1 | 0.042 | 0.028 | 0.017 |
| 74 | | | 107 | | |
| 0.12 | 0.14 | 0.066 | 0.037 | 0.02 | 0.023 |
| 80 | | | 126 | | |
| 0.12 | 0.1 | 0.095 | 0.014 | 0.029 | 0.044 |
| 76 | | | 80 | | |
| 0.11 | 0.071 | 0.12 | 0.027 | 0.025 | 0.033 |
| 120 | | | 27 | | |
| 0.072 | 0.1 | 0.075 | 0.042 | 0.026 | 0.013 |
| HER2 std_1 | | | HER2 std_1 | | |
| 0.079 | 0.088 | 0.063 | 0.026 | 0.024 | 0.023 |
| 111 | | | 116 | | |
| 0.1 | 0.025 | 0.092 | 0.06* | 0.029 | 0.019 |
| 121 | | | 104 | | |
| 0.12 | 0.068 | 0.026 | 0.028 | 0.015 | 0.027 |
| 106 | | | 97 | | |
| 0.083 | 0.062 | 0.068 | 0.014 | 0.033 | 0.022 |
| HER2 std_2 | | | 61 | | |
| 0.054 | 0.071 | 0.07 | 0.02 | 0.02 | 0.027 |
| 26 | | | 26 | | |
| 0.059 | 0.1 | 0.032 | 0.02 | 0.017 | 0.028 |
| 126 | | | 127 | | |
| 0.08 | 0.029 | 0.075 | 0 | 0.037 | 0.028 |
| 70 | | | 64 | | |
| 0.052 | 0.042 | 0.089 | 0.00729 | 0.031 | 0.025 |
| Mock | | | 121 | | |
| 0.032 | 0.059 | 0.089 | 0.035 | 0.01 | 0.017 |
| 24 | | | 24 | | |
| 0.1 | 0.046 | 0.027 | 0.028 | 0.013 | 0.019 |
| 27 | | | 130 | | |
| 0.066 | 0 | 0.098 | 0.02 | 0.018 | 0.021 |
| 20 | | | 106 | | |
| 0.061 | 0.048 | 0.039 | 0.016 | 0.017 | 0.023 |
| 127 | | | HER2 std_2 | | |
| 0.053 | 0.062 | 0.029 | 0.024 | 0.017 | 0.013 |
| 97 | | | 71 | | |
| 0.041 | 0.057 | 0.042 | 0 | 0.022 | 0.031 |
| 107 | | | 120 | | |
| 0.032 | 0.066 | 0.028 | 0.014 | 0.022 | 0.015 |
| 124 | | | 70 | | |
| 0.05 | 0.027 | 0.029 | 0.00858 | 0.029 | 0.00597 |
| 130 | | | 111 | | |
| 0.052 | 0.016 | 0.015 | 0 | 0.014 | 0.0081 |

### Example 5: Effects of IRES on Cytotoxicity

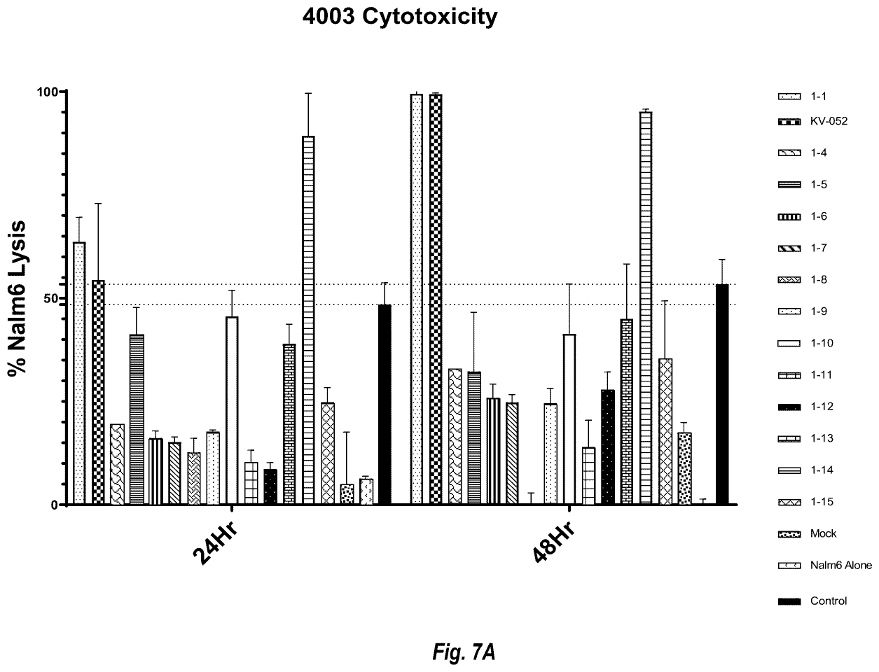

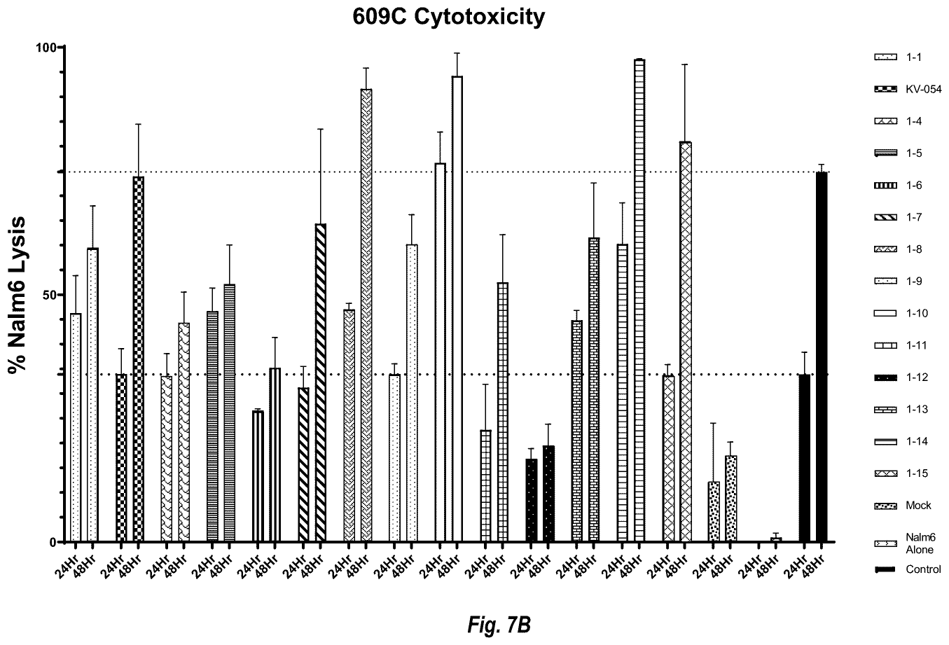

oRNA constructs comprising a base CD19 CAR construct in combination with different IRESes described in Table 1A (see, e.g., Table 1B), including the IRES sequences corresponding to SEQ ID NOs: 1, 4-15 were assessed for % Nalm6 lysis in two donors (Donor 4003 and Donor 609C). CD3+ T cells from two healthy donors (609C; 4003) were activated with aCD3/CD28 tetrameric complexes for 3 days and then electroporated with the CD19 CAR ORNA sequences. Nalm6 killing was assessed at 24 and 48 hours for each CD19 CAR base oRNA construct along with a mock negative control, Nalm6 alone, and the base alone. See FIGS. 7A-7B. Dotted horizontal lines reflect Nalm6 killing at 24- and 48-hour timepoints using the control CD19 CAR oRNA base construct (3276).

### Example 6: Cytotoxicity

### A. Cytotoxicity (69 Constructs)

CD3+ T cells from two healthy donors (609C; 4003) were activated with aCD3/CD28 tetrameric complexes for 3 days and then electroporated with CD19 CAR ORNA sequences. 74 total sequences including the 69 constructs of Table 5 were evaluated, comprising a combination of 5 CD19 CAR sequences comprising SEQ ID NOs: 19-23 (the 5 codon optimized sequences of Table 2A) and 18 IRES sequences (including controls). 24 hours post-electroporation, transfected T cells were co-cultured with Nalm6 cells for up to 48 hours. Nalm6 killing was assessed at 24 and 48 hours for each CD19 CAR oRNA construct and all constructs were rank ordered based on Nalm6 killing at 24 hours. Dotted horizontal lines reflect Nalm6 killing at 24 and 48 hour timepoints using the control CD19 CAR oRNA base construct (3276).

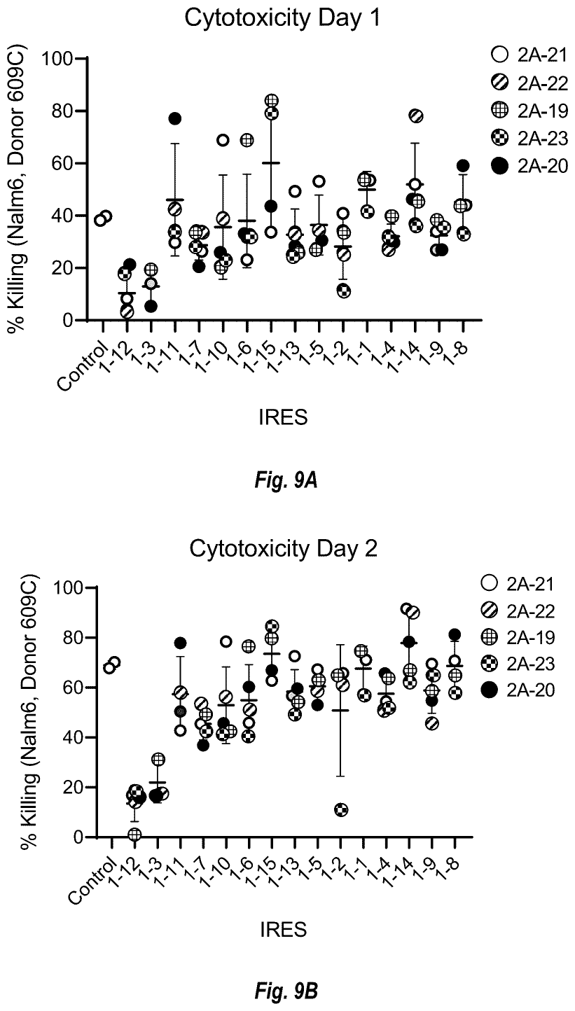

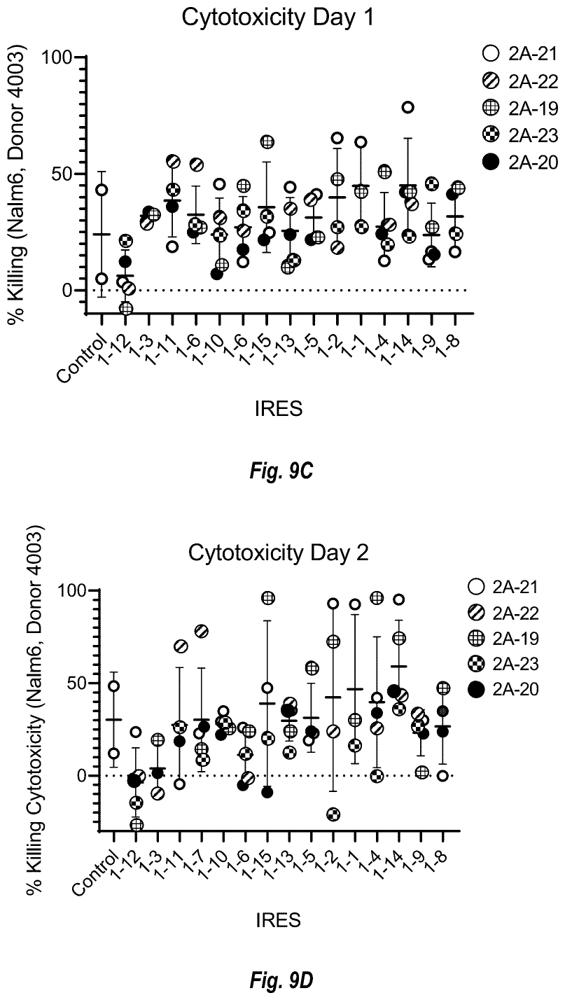

FIGS. 8A-8B and the tables below show a gradient across the constructs with certain constructs performing better than the base. FIG. 9A-9D present the same cytotoxicity data as FIGS. 8A-8B, but is organized by the IRESes in Table 1A (including IRESes comprising SEQ ID NOs: 1-15 of Table IA, and an IRES corresponding to a base CD19 control) and the expression sequences in Table 2A, for days 1 and 2 for the two donors.

TABLE
Donor 609C (cytotoxicity)

| Construct | | | | | | |
|---|---|---|---|---|---|---|
| number | 24 hours | | | 48 hours | | |
| 180 | 82.47 | | 94.26 | 99.83 | 88.28 | |
| 101 | 72.26 | | 81.07 | 97.48 | 90.96 | |
| 177 | 73.46 | 79.66 | | 99.3 | 99.86 | |
| 164 | 77.31 | 63.82 | | 99.42 | 94.53 | |
| 116 | 72.3 | 62.95 | | 99.48 | 93.29 | |
| 7 | 54.22 | | 69.98 | 97.14 | 71.58 | |
| 161 | 54.38 | 66.14 | | 97.65 | 97.55 | |
| 67 | 68.49 | 51.85 | | 98.96 | 82.72 | |
| 86 | 57.35 | 49.01 | | 91.98 | 87.07 | |
| 10 | 57.74 | | 45.42 | | 17.95 | 54.97 |
| 114 | | 44.83 | 56.02 | 79.97 | 69.84 | |
| 167 | 50.01 | | 46.4 | 96.99 | | 75.15 |
| 97 | | 40.88 | 54.2 | 73.19 | 61.54 | |
| 87 | 48.07 | | 46.87 | | 68.47 | 60.87 |
| 81 | 46.21 | 47.9 | | 94.59 | 88.67 | |
| 51 | 49.97 | 43.35 | | | 57.7 | 46.32 |
| 1 | 40.97 | 51.64 | | | 65.48 | 53.55 |
| 170 | | 38.78 | 52.68 | | 59.99 | 57.11 |
| 131 | 46.25 | 43.41 | | | 69.35 | 53.78 |
| 104 | 47.21 | | 42.14 | | 23.61 | 50.69 |
| 90 | 43.32 | | 43.42 | 96.65 | | 64.33 |
| 166 | | 43.33 | 43.35 | | 74.59 | 63.71 |
| 74 | | 37.07 | 49.22 | 64.39 | 63.58 | |
| 100 | 35.55 | 45.84 | | 94.07 | 80.59 | |
| 77 | | 33.67 | 47.04 | | 37.12 | 29.73 |
| 120 | 44.55 | | 34.14 | | 21.06 | 41.83 |
| 176 | 37.43 | | 39.09 | | 66.66 | 53.82 |
| 56 | 36.33 | | 38.22 | | 17.58 | 50.35 |
| 54 | 32.47 | 31.56 | 39.74 | | 43.57 | 40.91 |
| 64 | | 35.31 | 33.68 | 67.78 | 58.17 | |
| 17 | 30.28 | 36.02 | 36.52 | | 54.59 | 53.72 |
| 11 | 30.34 | 37.59 | | 81.38 | 66.48 | |
| 91 | 35.71 | 34.52 | 31.65 | | 64.43 | 55.96 |
| 126 | | 20.54 | 46.96 | 11.28 | | 10.29 |
| 171 | | 32.24 | 35.25 | 91.99 | | 70.07 |
| 36 | | 35.83 | 31.36 | | 66.75 | 40.24 |
| 31 | | 36.7 | 30.02 | | 48.7 | 39.85 |
| 57 | | 34.55 | 32.07 | 84.31 | 68.29 | |
| 14 | 30.75 | | 35.34 | | 55.87 | 44.05 |
| 130 | | 19.37 | 44.52 | 18.27 | 19.24 | 19.19 |
| 37 | 29.61 | 34.24 | | | 62.62 | 48.96 |
| 71 | 28.23 | | 34.26 | 77.87 | | 50.88 |
| 134 | | 29.23 | 32.53 | | 29.88 | 46.09 |
| 94 | | 28.51 | 33.15 | | 44.42 | 31.05 |
| 80 | | 30.98 | 29.93 | 54.84 | 44.52 | |
| 66 | 29.56 | | 30.69 | 76.9 | 72.33 | |
| 24 | | 11.57 | 48.39 | | 12.55 | 11.18 |
| 96 | | 29.77 | 29.91 | | 52.02 | 32.35 |
| 40 | 29.42 | | 28.99 | 74.42 | 59.57 | |
| 136 | 31.34 | 28.04 | 25.67 | 74.52 | 65.51 | |
| 137 | 24.52 | 28.72 | 28.08 | | 47.09 | 36.73 |
| 61 | | 26.37 | 26.82 | | 39.57 | 30.87 |
| 110 | | 24.19 | 24.62 | | 39.51 | 17.47 |
| 34 | 23.6 | 24.86 | | | 43.28 | 27.4 |
| 111 | 16.2 | | 29.21 | 45.69 | 59.32 | |
| 70 | 25.21 | 19.8 | | | 31.08 | 31.29 |
| 140 | 19.92 | 22.47 | | | 20.23 | 48.47 |
| 106 | 16.52 | | 24.97 | 64.57 | 50.56 | |
| 20 | | 13.03 | 23.51 | 20.73 | 13.61 | |
| 76 | 17.4 | 18.49 | | | 34.25 | 18.25 |
| 107 | 17.3 | 16.75 | | | 37.66 | 25.44 |
| 121 | | 15.41 | 18.29 | 16.45 | 22.58 | |
| 26 | | 10.96 | 22.16 | 14.97 | 14.86 | |
| 27 | 13.72 | 14.92 | | 57.7 | | 32.66 |
| 127 | | 2.7 | 12.85 | 3.5 | 3.11 | |
| 124 | | 3.15 | 10.52 | 18.67 | 15.48 | |
| mock | | 3.68 | 20.55 | 14.35 | 19.67 | 18.38 |
| Nalm6 only A | −6.09 | −1.05 | | 0.36 | | 1.56 |
| Nalm6 only B | 6.09 | 1.05 | | −0.36 | | −1.56 |
| PL3276.1* | | | | | | |
| PL3276.2 | 30.74 | | 37.05 | 75.91 | 73.75 | |

TABLE
Donor 4003 (cytotox)

| Construct | Donor 4003 | | | | | |
|---|---|---|---|---|---|---|
| Number | 24 hours | | | 48 hours | | |
| 161 | 82.07 | | 96.59 | 95.34 | 94.55 | 95.66 |
| 74 | 72.42 | 93.48 | | 95.92 | 99.87 | |
| 100 | 82.18 | 73.54 | | | −0.82 | −15.45 |
| 177 | 72.49 | | 72.33 | 92.42 | 98.85 | 98.39 |
| 114 | 66.9 | 60.59 | | 88.82 | 82.94 | |
| 1 | 65.83 | 56.91 | 68.18 | | 99.96 | 99.03 |
| 167 | 42.45 | | 66.65 | | 99.9 | 97.3 |
| 11 | 41.33 | | 67.52 | | 99.61 | 99.19 |
| 120 | | 42.12 | 62.76 | 29.21 | | 32.94 |
| 67 | | 53.2 | 49.86 | 36.92 | 26.15 | |
| 54 | | 49.35 | 49.85 | 46.19 | | 30.5 |
| 166 | | 43.19 | 52.24 | 47.79 | 48.65 | |
| 101 | 51.86 | 39.2 | 45.65 | 49.91 | 32.88 | |
| 27 | | 47.8 | 42.77 | | 15.45 | 17.24 |
| 134 | 50.02 | 37.41 | | 59.67 | 40.79 | |
| 116 | 49.3 | 37.15 | | 41.38 | | 27.21 |
| 7 | 36.2 | 42.97 | 49.86 | 71.98 | 0.2 | 19.92 |
| 164 | | 38.03 | 46.68 | | 42.48 | 34.38 |
| 70 | | 33.71 | 49.2 | 31.1 | | 28.42 |
| 51 | 47.51 | 41.68 | 34.37 | 42.39 | | 22.13 |
| 87 | 38.23 | 42.17 | | 52.26 | 53.98 | |
| 131 | 35.64 | 42.33 | | 54.41 | | 35.62 |
| 97 | | 39.91 | 37.95 | | 17.42 | 30.36 |
| 80 | 35.32 | 42.43 | | | 20.7 | 21.77 |
| 37 | | 39.48 | 36.72 | | 99.82 | 99.81 |
| 24 | | 31.64 | 42.49 | 16.79 | | 5.88 |
| 86 | 30.49 | 40.88 | | 53.23 | | 20.11 |
| 26 | 31.09 | 24.27 | 45.6 | 21.73 | | 25.58 |
| 176 | | 36.89 | 27.19 | −39.29 | | −1.09 |
| 104 | 36.13 | 29.29 | 30.11 | | 27.42 | 18.1 |
| 17 | | 40.86 | 22.09 | | 67.06 | 55.07 |
| 57 | 29.99 | 31 | | 46.16 | | 46.3 |
| 90 | | 30.29 | 27.67 | | 32.92 | 28.97 |
| 130 | 37.81 | 19.66 | | 12.68 | | 5.09 |
| 10 | 27.93 | 31.85 | 24.06 | 24.62 | 18.81 | |
| 180 | 23.14 | 31.97 | | 56.71 | | 24.62 |
| 136 | 25.31 | | 29.72 | 43.45 | 44.03 | |
| 20 | 21.97 | 24.62 | 35.85 | −56.38 | | −9.6 |
| 171 | 26.48 | 27.24 | 20.75 | | 45.25 | 25.48 |
| 34 | 26.22 | 22.48 | | | 23.08 | 16.9 |
| 36 | 28.52 | 19.96 | 24.21 | 38.59 | 35.78 | |
| 77 | 24.08 | 20.69 | | | 24.45 | 20.86 |
| 56 | 18.5 | 25.39 | 21.38 | 28.7 | 22.11 | 21.25 |
| 66 | 21.96 | 21.16 | | −24.9 | | −3.67 |
| 170 | 17.4 | 22.07 | | 42.8 | 42.58 | |
| 64 | 20.12 | 19.01 | | | 16.55 | 8.62 |
| 31 | 21.26 | 17.77 | | 37.89 | | 27.69 |
| 126 | | 8.62 | 29.94 | | 20.47 | 20.84 |
| 110 | 21.86 | 15.09 | | | 17.68 | 22.17 |
| 91 | 17.42 | 18.01 | | | 27.14 | 22.06 |
| 140 | 16.51 | 16.48 | | | 22.75 | 20.98 |
| 76 | 16.59 | 15.86 | | | 25.11 | 23.59 |
| 61 | | 17.37 | 14.91 | 29.58 | 25.06 | 23.1 |
| 96 | 16.69 | 16.26 | 13.01 | | 27.07 | 26.36 |
| 71 | 14.31 | | 16.06 | 26.14 | 23.43 | |
| 94 | 14.17 | | 14.13 | | 29.96 | 29.18 |
| 40 | | 18.59 | 9.03 | −15.65 | | −0.65 |
| 106 | 14.66 | 12.69 | | | 19.27 | 11.37 |
| 81 | | 15.11 | 10.29 | −16.59 | | −0.47 |
| 107 | 8.08 | 11.77 | 13.21 | 29.86 | | 27.01 |
| 14 | | 16.68 | 5.03 | 35.76 | 25.75 | |
| 111 | 8.1 | 12.36 | | | 18.59 | 9.42 |
| 121 | 9.76 | 7.55 | | 30.91 | | 24.92 |
| 137 | 6.3 | | 9.98 | 29.17 | 26.4 | |
| 124 | 11.62 | 0.53 | | | 11.61 | 28.24 |
| 127 | −4.27 | −3.68 | −14.9 | | 0.37 | −8.26 |
| Mock | −4 | 13.88 | | 14.87 | 18.18 | 19.52 |
| Nalm6 Alone_1 | 5.93 | 6.74 | | −0.39 | −0.04 | −6.8 |
| Nalm6 Alone_2 | −5.93 | −6.74 | | 0.39 | 0.04 | |
| 3276_1 | | 44.73 | 52.2 | 57.63 | 49.11 | |

B. Comparison of Cytokine Production and Cytotoxicity

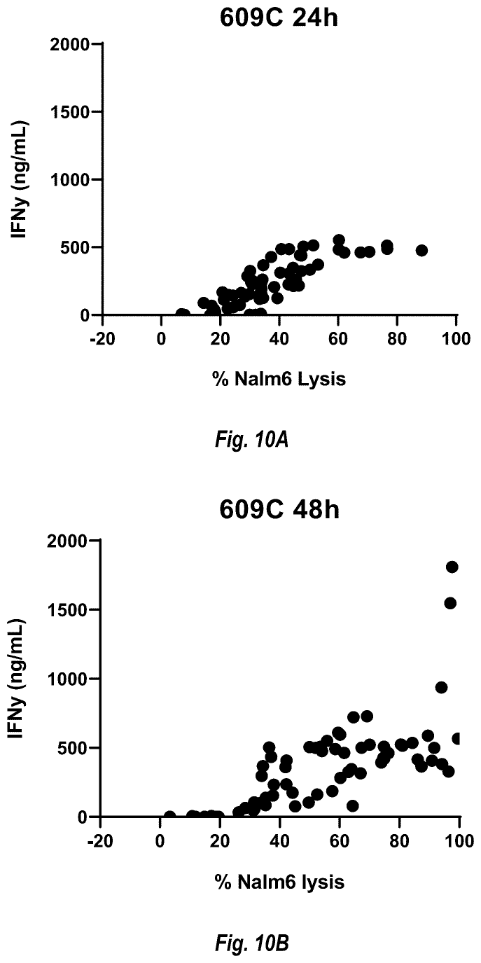

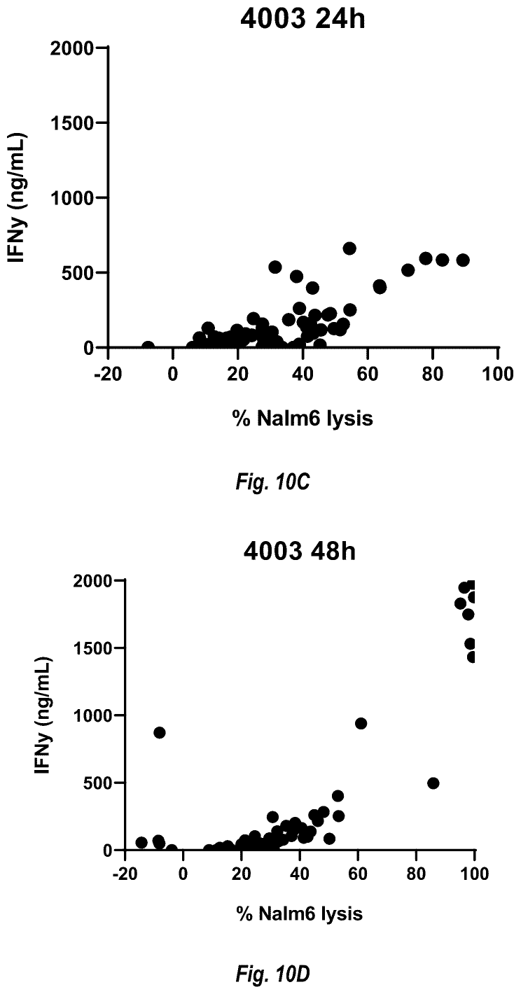

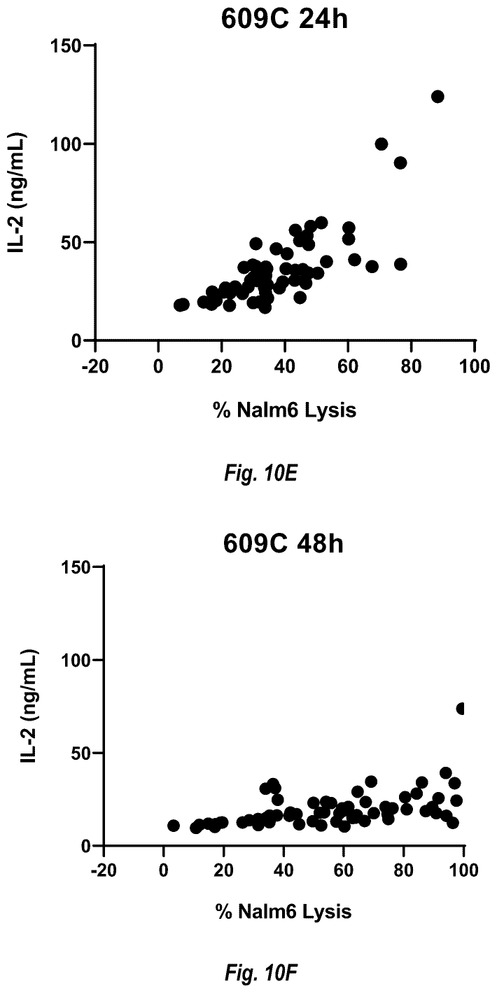

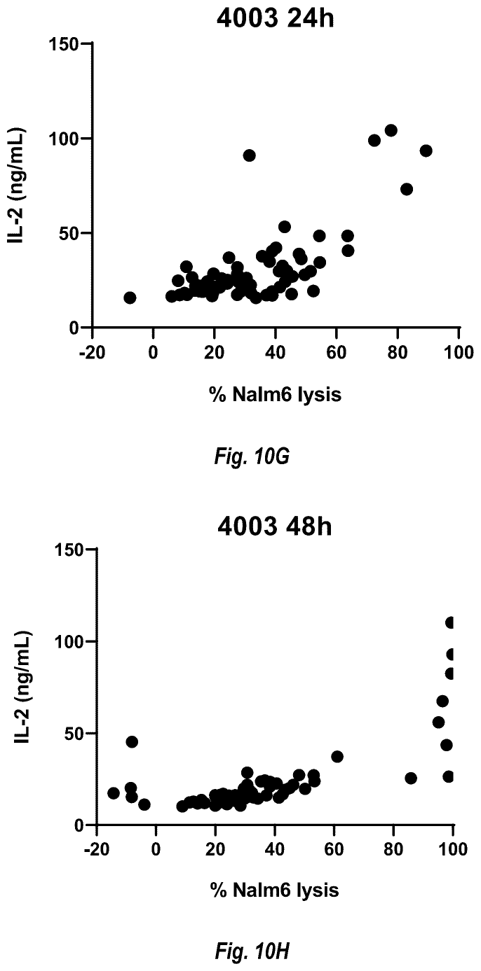

Coculture supernatants were collect and analyzed using Meso Scale Discovery (MSD) (Agilent Technologies, Santa Clara, CA) for IFNγ (FIGS. 10A-D) and IL-2 expression (FIGS. 10E-H). CD3+ T cells from two healthy donors (609C, first row; 4003, second row) were activated with αCD3/CD28 tetrameric complexes for 3 days and then electroporated with CD19 CAR ORNA sequences. 74 total sequences were evaluated, comprising a combination of 5 CD19 CAR sequences comprising SEQ ID NOs: 19-23 (the 5 codon optimized sequences of Table 2A) and 18 IRES sequences (which include IRES sequences corresponding to SEQ ID NOs: 1-15 in Table 1A) (see, e.g., Table 1B). 24 hours post-electroporation, transfected T cells were co-cultured with Nalm6 cells for up to 48 hours. At 24-hour and 48-hour timepoints, supernatants were collected and IL-2 and IFN-γ amounts were quantified via MSD.

### Example 7: Cytotoxicity (12 Constructs of Table 6)

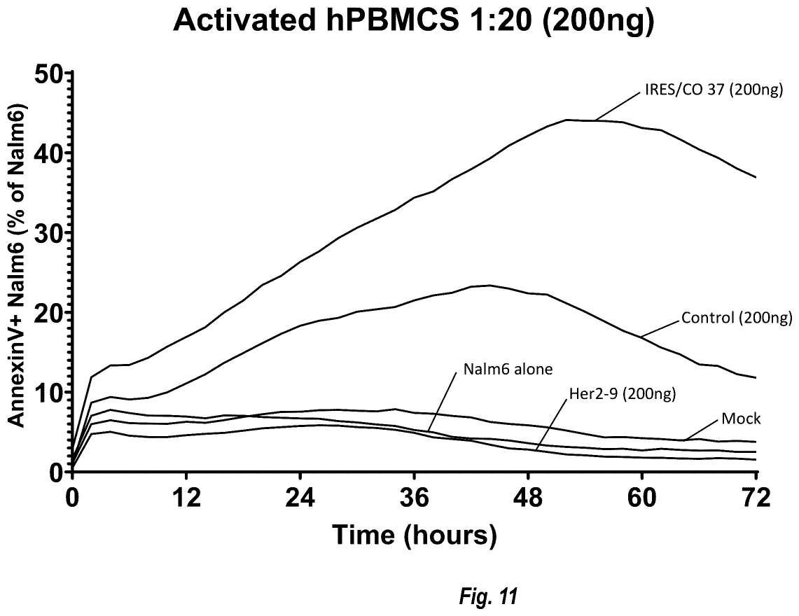

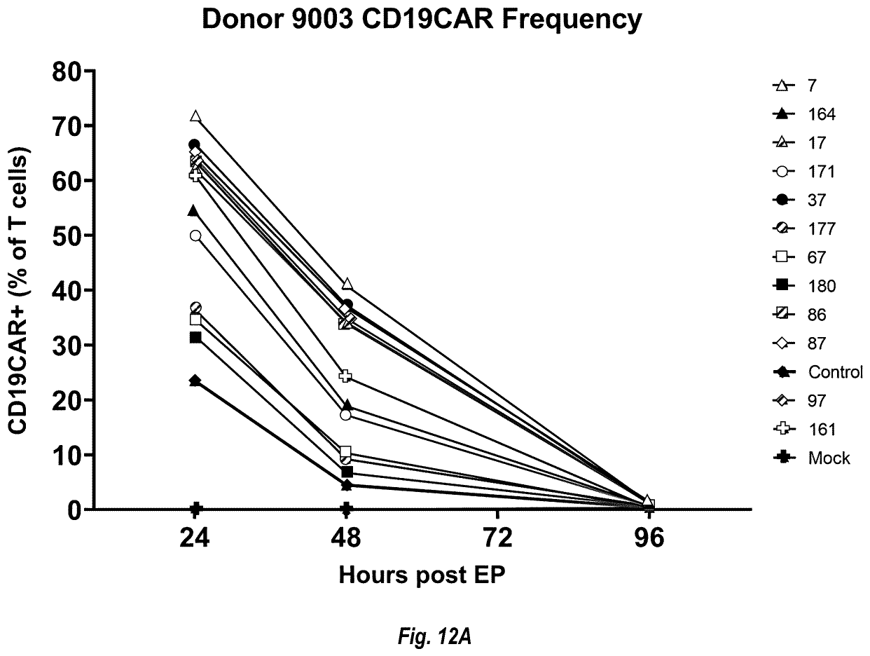

Cytotoxicity was assessed for the 12 circular RNA constructs of Table 6 by administering the circular RNA constructs over 3 days (24, 48, 72 hours) and assessing cytotoxicity at 2 E: T up to 72 hours, luminescence and Incucyte based cytotoxicity readouts, and cytokine readouts from luminescence plates. CD3+ T cells were activated with aCD3/CD28 tetrameric complexes for 3 days and then transfected with PEG-modified lipid 86 of Table 3 (lipid of Formula I) delivering a HER2 construct (HER2_9, Table 9), a base CD19 CAR construct (3276 comprising SEQ ID NO: 21) containing a non-optimized CAR sequence, or the CD19 CAR construct of IRES/CO Clone #37 (Table 5) of SEQ ID NO: 52. 24 hours post-transfection, T cells were co-cultured with GFP+Nalm6 B-ALL cells at 1:20 T cell to Nalm6 ratio. Annexin V was added to mark dead cells. C0-cultures were imaged via Incucyte every 2 hours to identify Nalm6, dead/dying cells, and dead/dying Nalm6 cells. Results are plotted as percentage of dead/dying Nalm6 cells divided by total Nalm6 cells. See FIG. 11. These data show the oRNA construct comprising the sequence of SEQ ID NO: 43 as compared to a base CD19 CAR construct (3276) and as compared to a HER2 oRNA with a mock negative control and as compared to Nalm6 alone.

### Example 8: In Vitro Experimentation (12 Constructs)

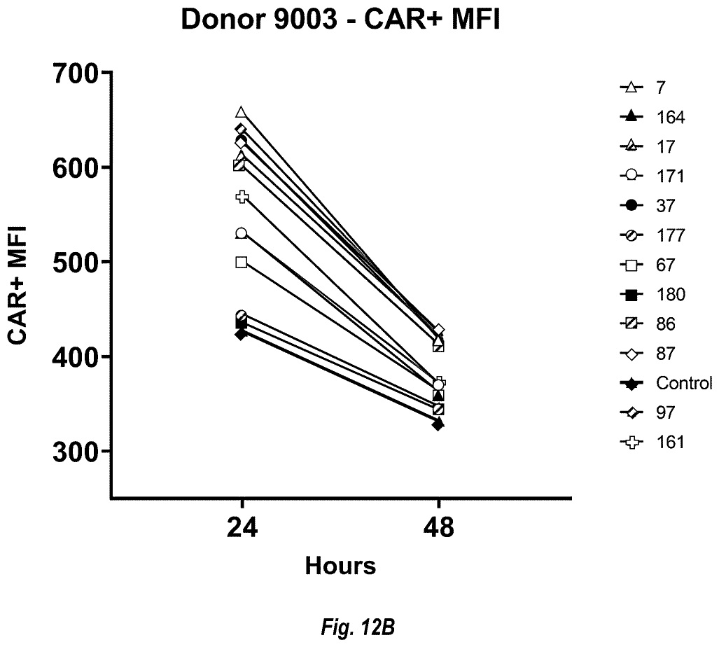

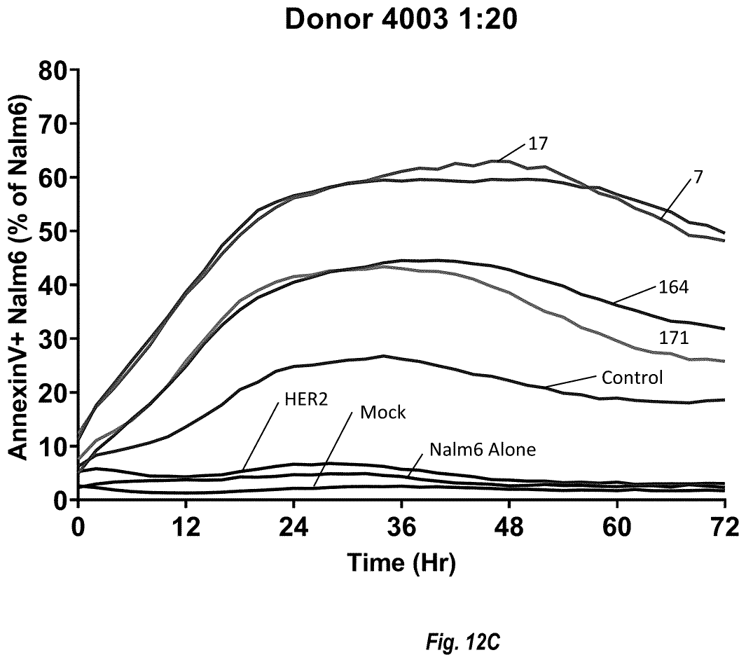

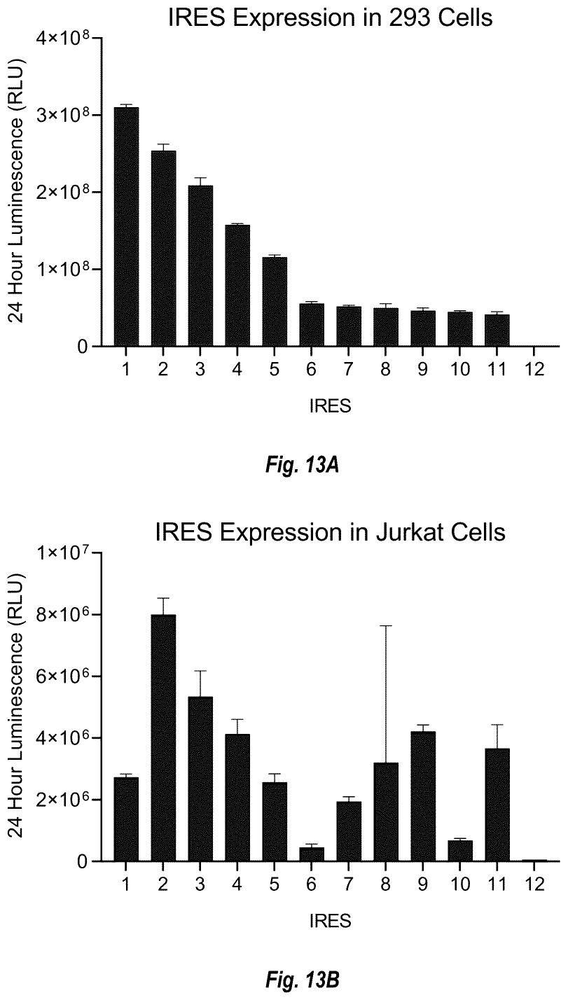

Twelve oRNA constructs were designed to comprise a combination of IRESes and codon optimized sequences (Table 6) and tested against (1) a base oRNA construct comprising a non-optimized CD19 CAR expression sequence and a kobuvirus IRES for expression, and (2) an oRNA construct comprising a HER2 CAR expression sequence. A mock construct (vehicle control) was used as a negative control. oRNA constructs were electroporated into activated T cells from a single donor (Donor 9003) using standard practices in the art. The cells were then incubated overnight. See FIG. 12A, showing % of T cells over 96 hours.

Expression of the CD19 CAR was collected 24, 48, 72, and 96 hours post electroporation of the T cells. 24 hours following electroporation, the T cells were stained with either Live/Dead Near IR stain, Fc block, and either biotinylated anti-FMC63, and either biotinylated anti-HER2 antibody, CD3, Streptavidin antibody and then incubated. The cells were then washed with a stain buffer.

The same oRNA constructs from above, including the base oRNA and ORNA construct encoding for HER2, along with the mock construct were analyzed for cytotoxicity. Said ORNA constructs were electroporated in either T cell or Nalm6 plates (Nalm 6 only control plates). T cells were from two donors (Donor 4003 and Donor 9003). The T cells were then cocultured with Nalm6 cells at a E: T ratio of 1:20. Cytotoxicity was analyzed using FACS at 24 hours and 48 hours post electroporation. Both the cocultured cells along with the Nalm6 only cells were dyed with Annexin V Red and then scanned in a standard tissue culture incubator (Incucyte). See FIG. 12B (CAR+MFI over 48 hours post electroporation) and 12C (Annexin V+Nalm6 over 72 hours for construct nos. 7, 164, 17, and 171 as compared to a base CD19 and HER2 control, mock negative control and Nalm6 alone).

### Example 9: IRESCO Constructs Enhance In Vivo Tumor Efficacy

NSG mice were engrafted with Nalm6-Luciferase (Luc) tumor cells and 4 days later were engrafted with human PBMCs. Starting on Day 5, the mice were treated 4 times every other day with vehicle (PBS), control, or anti-CD19 LNP-oCAR compounds at doses of 1.0 mg/kg, 0.3 mg/kg, and 0.1 mg/kg. PBS control is representative of mice with engrafted Nalm6-luc tumor cells only and treated with vehicle. LNP used herein comprised PEG-ionizable lipid 86 of Table 3. Animals were then whole-body imaged via IVIS to monitor luciferase expression from Nalm6 cells. Nalm6 tumor burden is plotted as Total Flux (photons/second over a region of interest) of the liver, spleen, kidney, lung, and heart of luciferase expression at each imaging timepoint.

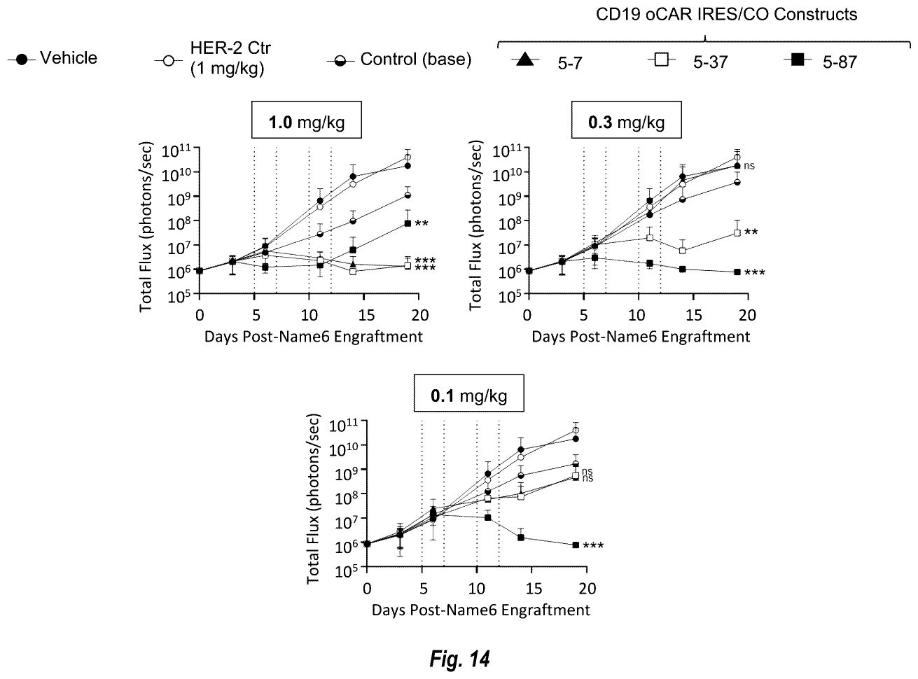

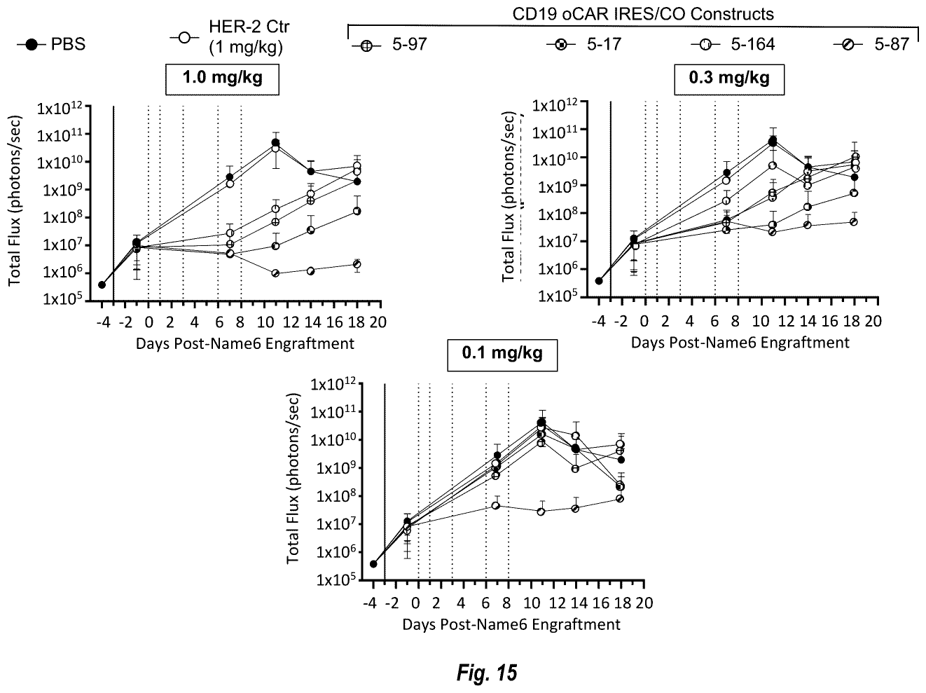

FIG. 14 shows that CD19 oCAR constructs comprising SEQ ID NOs: 50, 52, and 55 (IRES/CO clone #s 7, 37, and 87) have higher anti-tumor efficacy compared to a base CD19 CAR control, PBS, and a HER2 control construct. FIG. 15 shows that CD19 oCAR constructs comprising SEQ ID NOs: 56, 51, 58, and 55 (IRES/CO clone #s 97, 17, 164, and 87) have higher anti-tumor efficacy compared to PBS and a HER2 control construct.

### Example 10: Anti-Tumor Efficacy is Observed with all Lipids after 4 Doses of LNP/oCAR

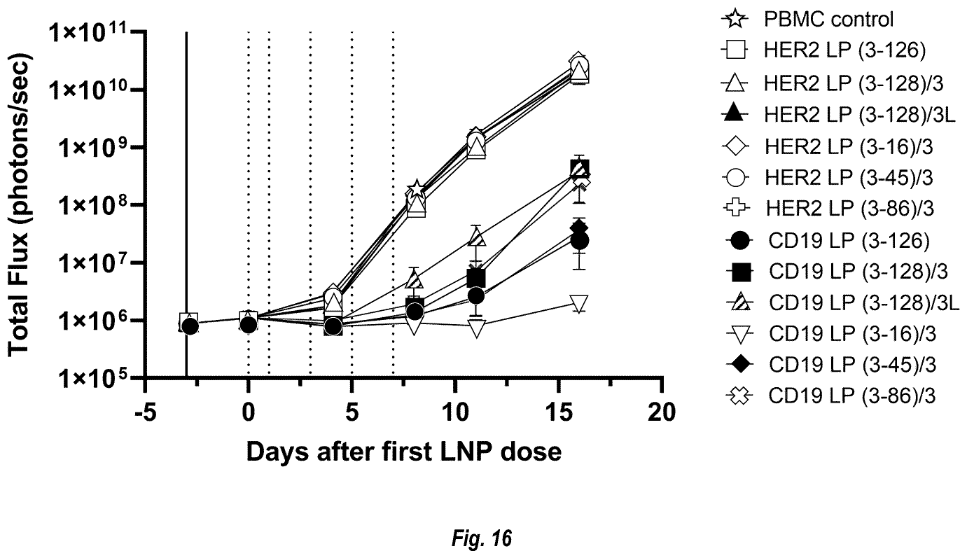

Total Flux (photons/second) was observed over time for oRNA constructs with expression sequences directed to HER2 and CD19 (see, e.g., Table 1B) and to assess the effect of anti-tumor efficacy after 4 doses using different lipids of LNP-circRNAs, where the effects of PEG lipids in the transfer vehicle was also assessed. NSG mice were engrafted with Nalm6-Luciferase (Luc) tumor cells and 3 days later were engrafted with human PBMCs. Starting on Day 1, the mice were treated 4 times every other day with vehicle (PBS) or anti-CD19 LNP-oCAR compounds. LNPs delivering the oCAR constructs comprised of PEG-modified lipids of control lipids (11 and 62) and lipids of Formula I, including lipids 16, 45, and 86 of Table 3) at an ionizable lipid to phosphate ratio (IL: P) of 5.7. The ionizable lipid: helper lipid: cholesterol: PEG-lipid molar ratio of these LNPs was 50:10:38.5:1.5. The formulation of the LNPs are further detailed below. PBS control is representative of mice with engrafted Nalm6-luc tumor cells only and treated with vehicle. Animals were then whole-body imaged via IVIS to monitor luciferase expression from Nalm6 cells. Nalm6 tumor burden is plotted as Total Flux of luciferase expression at each imaging timepoint. See FIG. 16.

| | | | | Ionizable | | | | |
|---|---|---|---|---|---|---|---|---|
| | | | | Lipid:Helper | | | RNA | |
| | | | | Lipid:Choles- | | Z- | Encapsulation | |
| | Ionizable | Helper | PEG- | terol:PEG-lipid | HER2/ | Average | Efficiency | |
| Formulation | Lipid | Lipid | Lipid | (Mol %) | CD19 | (nm) | (%) | PDI |
| 3-126 | Lipid 126 | DSPC | DMG- | 50:10:38.5:1.5 | HER2 | 93 | 93 | 0.03 |
| [5.7A] | of Table 3 | | PEG 2000 | | CD19 | 81 | 96 | 0.07 |
| (3-128)/3L | Lipid 128 | DSPC | DMG- | 50:10:38.5:1.5 | HER2 | 97 | 97 | 0.04 |
| [5.7A] | of Table 3 | | PEG 2000 | | CD19 | 82 | 95 | 0.02 |
| (3-128)/3 | Lipid 128 | DSPC | DMG- | 50:10:38.5:1.5 | HER2 | 97 | 97 | 0.14 |
| [5.5A] | of Table 3 | | PEG 2000 | | CD19 | 106 | 96 | 0.15 |
| (3-16)/3 | Lipid 16 of | DSPC | DMG- | 50:10:38.5:1.5 | HER2 | 91 | 91 | 0.06 |
| [5.5A] | Table 3 | | PEG 2000 | | CD19 | 65 | 96 | 0.05 |
| (3-45)/3 | Lipid 45 of | DSPC | DMG- | 50:10:38.5:1.5 | HER2 | 97 | 97 | 0.03 |
| [5.5A] | Table 3 | | PEG 2000 | | CD19 | 67 | 96 | 0.05 |
| (3-86)/3 | Lipid 86 of | DSPC | DMG- | 50:10:38.5:1.5 | HER2 | 94 | 94 | 0.05 |
| [5.5A] | Table 3 | | PEG 2000 | | CD19 | 81 | 94 | 0.03 |

### Example 11: Circular RNA Constructs Comprising an Anti-HER2 Binder

Additional circular RNA constructs comprising an anti-HER2 binder were designed and assessed. Certain of the circular RNA constructs in Table 9 include a miR-122 site.

TABLE 9
Additional Codon-IRES Constructs (HER2)

| IRES/CO | IRES | Codon NT | |
|---|---|---|---|
| Clone # | SEQ ID NO: | SEQ ID NO: | |
| HER2_1 | 16 | 101 | |
| HER2_2 | 17 | 101 | Includes miR-122 site: |
| | | | SEQ ID NO: 200 |
| HER2_3 | 8 | 101 | |
| HER2_4 | 18 | 101 | |
| HER2_5 | 16 | 102 | |
| HER2_6 | 17 | 102 | Includes miR-122 site: |
| | | | SEQ ID NO: 200 |
| HER2_7 | 8 | 102 | |
| HER2_8 | 18 | 102 | |
| HER2_9 | 17 | 132 | |
| HER2_10 | 17 | 133 | |

### A. Cytotoxicity Assay

Two oRNA constructs (HER2_9 and HER2_10 in Table 9) were designed that comprise a combination of IRESes and expression sequences directed to HER2. The IRES-CO sequences for the constructs are set forth in SEQ ID NOs: 136 and 137:

HER2 9:
(SEQ ID NO: 136)
GTGGCCACGCCCGGGCCACCGATACTTCCCTTCACTCCTTCGGGACTGTTGGGGA
GGAACACAACAGGGCTCCCCTGTTTTCCCATTCCTTCCCCCTTTTCCCAACCCCAA
CCGCCGTATCTGGTGGCGGCAAGACACACGGGTCTTTCCCTCTAAAGCACAATTG
TGTGTGTGTCCCAGGTCCTCCTGCGTACGGTGCGGGAGTGCTCCCACCCAACTGT
TGTAAGCCTGTCCAACGCGTCGTCCTGGCAAGACTATGACGTCGCATGTTCCGCT
GCGGATGCCGACCGGGTAACCGGTTCCCCAGTGTGTGTAGTGCGATCTTCCAGGT
CCTCCTGGTTGGCGTTGTCCAGAAACTGCTTCAGGTAAGTGGGGTGTGCCCAATC
CCTACAAAGGTTGATTCTTTCACCACCTTAGGAATGCTCCGGAGGTACCCCAGCA
ACAGCTGGGATCTGACCGGAGGCTAATTGTCTACGGGTGGTGTTTCCTTTTTCTTT
TCACACAACTCTACTGCTGACAACTCACTGACTATCCACTTGCTCTGTCACGATGG
CTCTGCCTGTGACAGCTCTGCTGCTGCCTCTGGCTCTGCTTCTGCATGCCGCCAGA
CCTGACATCCAGATGACTCAGAGCCCCAGCAGCCTGTCTGCCTCTGTGGGAGACA
GAGTGACAATTACCTGCCGGGCCAGCCAGGATGTGAATACTGCTGTCGCCTGGTA
TCAACAAAAGCCTGGCAAGGCCCCTAAGCTCCTGATCTACAGCGCCAGCTTTCTG
TACAGCGGCGTGCCCAGCAGATTCTCCGGAAGCAGAAGCGGCACAGATTTCACA
CTGACCATAAGCAGCCTGCAGCCAGAGGATTTCGCCACCTACTATTGCCAGCAGC
ACTACACCACACCTCCAACCTTTGGCCAGGGCACCAAGGTCGAGATTAAGAGAA
CAGGCAGCACATCTGGCTCTGGCAAACCTGGATCTGGCGAGGGCTCTGAAGTCCA
GCTGGTGGAATCTGGCGGAGGACTGGTTCAACCTGGCGGCTCTCTGAGACTGTCT
TGTGCCGCCTCCGGCTTCAACATCAAGGACACCTACATCCACTGGGTCCGACAAG
CCCCAGGCAAAGGACTTGAGTGGGTCGCCAGGATCTACCCCACCAACGGCTACA
CCAGATACGCCGACTCTGTGAAGGGCAGATTCACCATCTCTGCCGACACCAGCAA
GAATACCGCCTACCTGCAGATGAACTCCCTGAGAGCCGAAGATACCGCTGTGTAT
TACTGTTCCAGATGGGGAGGCGACGGCTTCTACGCCATGGATGTTTGGGGCCAAG
GCACCCTCGTGACCGTTTCTTCTATCGAAGTGATGTACCCTCCACCTTACCTGGAC
AACGAGAAGTCCAACGGCACCATCATCCACGTGAAGGGCAAGCACCTGTGTCCT
TCTCCACTGTTCCCCGGACCTAGCAAGCCTTTCTGGGTGCTCGTTGTTGTTGGCGG
CGTGCTGGCCTGTTACTCTCTGCTGGTTACCGTGGCCTTCATCATCTTTTGGGTCC
GAAGCAAGCGGAGCCGGCTGCTGCACTCCGACTACATGAACATGACCCCTAGAC
GGCCCGGACCAACCAGAAAGCACTACCAGCCTTACGCTCCTCCTAGAGACTTCGC
CGCCTACCGGTCCAGAGTGAAGTTCAGCAGATCCGCCGATGCTCCCGCCTATCAG
CAGGGCCAAAACCAGCTGTACAACGAGCTGAACCTGGGGAGAAGAGAAGAGTA
CGACGTGCTGGACAAGCGGAGAGGCAGAGATCCTGAAATGGGCGGCAAGCCCAG
ACGGAAGAATCCTCAAGAGGGCCTGTATAATGAGCTGCAGAAAGACAAGATGGC
CGAGGCCTACAGCGAGATCGGAATGAAGGGCGAGCGCAGAAGAGGCAAGGGAC
ACGATGGACTGTACCAGGGCCTGAGCACCGCCACCAAGGATACCTATGATGCCCT
GCACATGCAGGCCCTGCCTCCAAGA
HER_10:
(SEQ ID NO: 137)
GTGGCCACGCCCGGGCCACCGATACTTCCCTTCACTCCTTCGGGACTGTTGGGGA
GGAACACAACAGGGCTCCCCTGTTTTCCCATTCCTTCCCCCTTTTCCCAACCCCAA
CCGCCGTATCTGGTGGCGGCAAGACACACGGGTCTTTCCCTCTAAAGCACAATTG
TGTGTGTGTCCCAGGTCCTCCTGCGTACGGTGCGGGAGTGCTCCCACCCAACTGT
TGTAAGCCTGTCCAACGCGTCGTCCTGGCAAGACTATGACGTCGCATGTTCCGCT
GCGGATGCCGACCGGGTAACCGGTTCCCCAGTGTGTGTAGTGCGATCTTCCAGGT
CCTCCTGGTTGGCGTTGTCCAGAAACTGCTTCAGGTAAGTGGGGTGTGCCCAATC
CCTACAAAGGTTGATTCTTTCACCACCTTAGGAATGCTCCGGAGGTACCCCAGCA
ACAGCTGGGATCTGACCGGAGGCTAATTGTCTACGGGTGGTGTTTCCTTTTTCTTT
TCACACAACTCTACTGCTGACAACTCACTGACTATCCACTTGCTCTGTCACGATGG
CTCTGCCTGTGACAGCTCTGCTGCTGCCTCTGGCTCTGCTTCTGCATGCCGCCAGA
CCTGACATCCAGATGACTCAGAGCCCCAGCAGCCTGTCTGCCTCTGTGGGAGACA
GAGTGACAATTACCTGCCGGGCCAGCCAGGATGTGAATACTGCTGTCGCCTGGTA
TCAACAAAAGCCTGGCAAGGCCCCTAAGCTCCTGATCTACAGCGCCAGCTTTCTG
TACAGCGGCGTGCCCAGCAGATTCTCCGGAAGCAGAAGCGGCACAGATTTCACA
CTGACCATAAGCAGCCTGCAGCCAGAGGATTTCGCCACCTACTATTGCCAGCAGC
ACTACACCACACCTCCAACCTTTGGCCAGGGCACCAAGGTCGAGATTAAGAGAA
CAGGCAGCACATCTGGCTCTGGCAAACCTGGATCTGGCGAGGGCTCTGAAGTCCA
GCTGGTGGAATCTGGCGGAGGACTGGTTCAACCTGGCGGCTCTCTGAGACTGTCT
TGTGCCGCCTCCGGCTTCAACATCAAGGACACCTACATCCACTGGGTCCGACAAG
CCCCAGGCAAAGGACTTGAGTGGGTCGCCAGGATCTACCCCACCAACGGCTACA
CCAGATACGCCGACTCTGTGAAGGGCAGATTCACCATCTCTGCCGACACCAGCAA
GAATACCGCCTACCTGCAGATGAACTCCCTGAGAGCCGAAGATACCGCTGTGTAT
TACTGTTCCAGATGGGGAGGCGACGGCTTCTACGCCATGGATGTTTGGGGCCAAG
GCACCCTCGTGACCGTTTCTTCTACCACCACACCAGCTCCTCGGCCTCCAACTCCT
GCTCCTACAATTGCCAGCCAGCCTCTGTCTCTGAGGCCCGAAGCTTGTAGACCTG
CTGCTGGCGGAGCCGTGCATACAAGAGGACTGGATTTCGCCTGCGACATCTACAT
CTGGGCTCCTCTGGCCGGAACATGTGGCGTTCTGCTGCTGAGCCTGGTCATCACC
CTGTACTGTAAGCGGGGCAGAAAGAAGCTGCTGTACATCTTCAAGCAGCCCTTCA
TGCGGCCCGTGCAGACCACACAAGAGGAAGATGGCTGCTCCTGCAGATTCCCCG
AGGAAGAAGAAGGCGGCTGCGAGCTGAGAGTGAAGTTCAGCAGATCCGCCGATG
CTCCCGCCTATCAGCAGGGCCAAAACCAGCTGTACAACGAGCTGAACCTGGGGA
GAAGAGAAGAGTACGACGTGCTGGACAAGCGGAGAGGCAGAGATCCTGAAATG
GGCGGCAAGCCCAGACGGAAGAATCCTCAAGAGGGCCTGTATAATGAGCTGCAG
AAAGACAAGATGGCCGAGGCCTACAGCGAGATCGGAATGAAGGGCGAGCGCAG
AAGAGGCAAGGGACACGATGGACTGTACCAGGGCCTGAGCACCGCCACCAAGGA
TACCTATGATGCCCTGCACATGCAGGCCCTGCCTCCAAGA

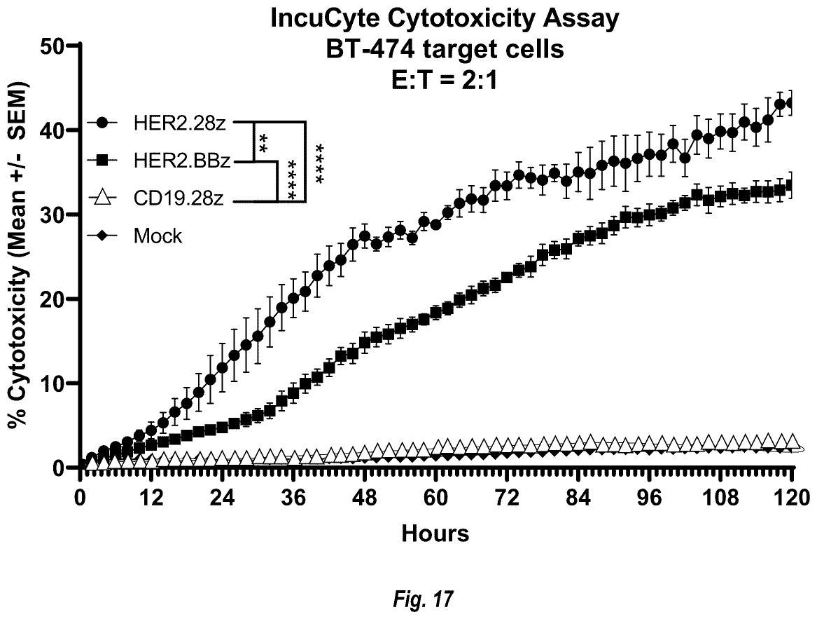

The two oRNA constructs HER2_9 and HER2_10 were tested against the control CD19 CAR oRNA base construct 3276 (codon optimized) and a mock negative control in an IncuCyte Cytotoxicity assay. Activated T cells (from BT-474 cell line) from a single donor (9003) were transfected in vitro with LNP-oCAR (200 ng/0.1e6 T cells). 24 hours after transfection, the CAR-T cells were evaluated for expression & co-cultured with target cells (2:1 E: T) over time for five days. See FIG. 17. Both HER2_9 and HER2_10 demonstrate cytotoxic function in a target-dependent manner.

B. HER2 and CD19 oCAR Cytotoxicity

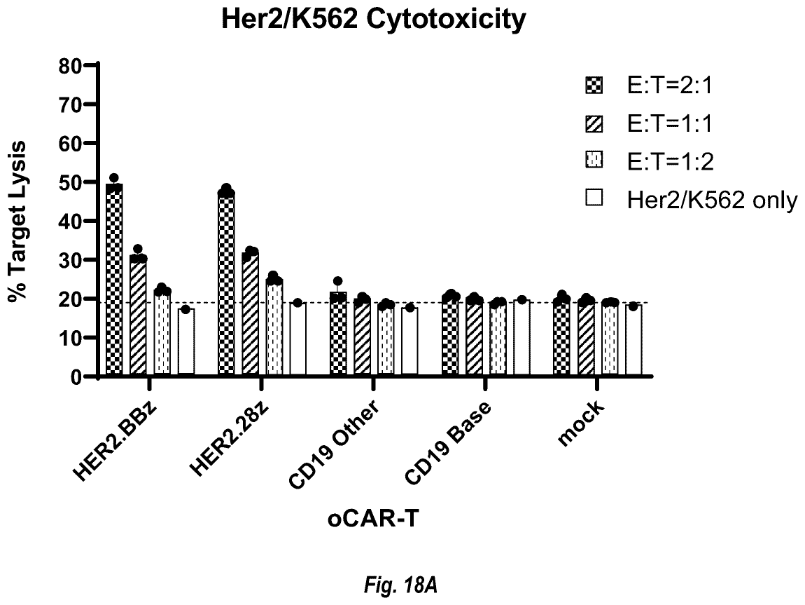

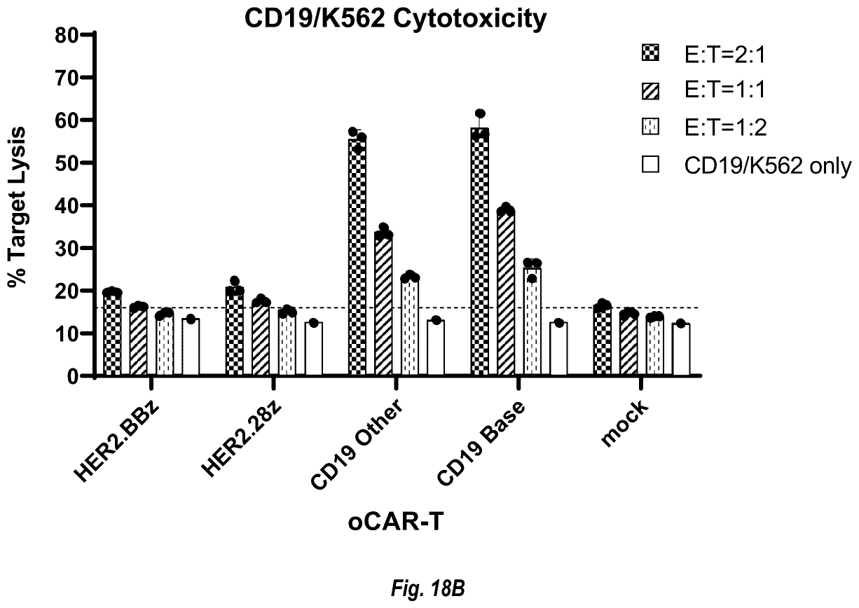

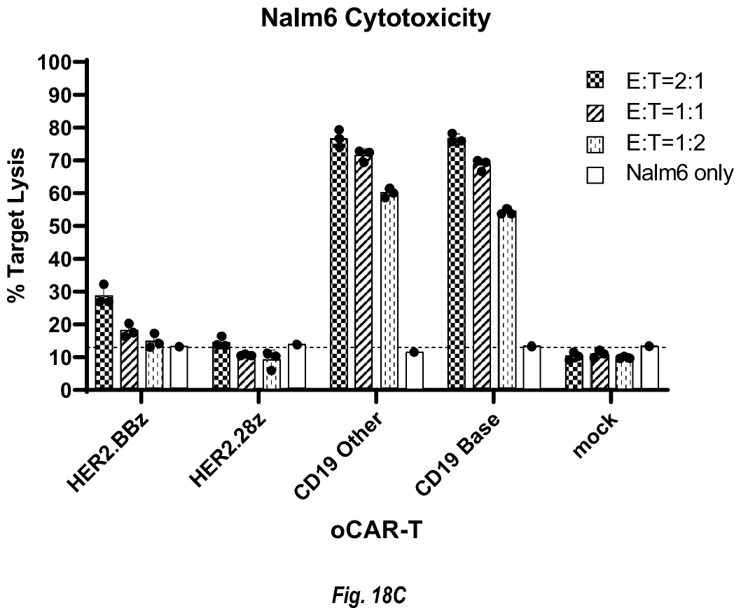

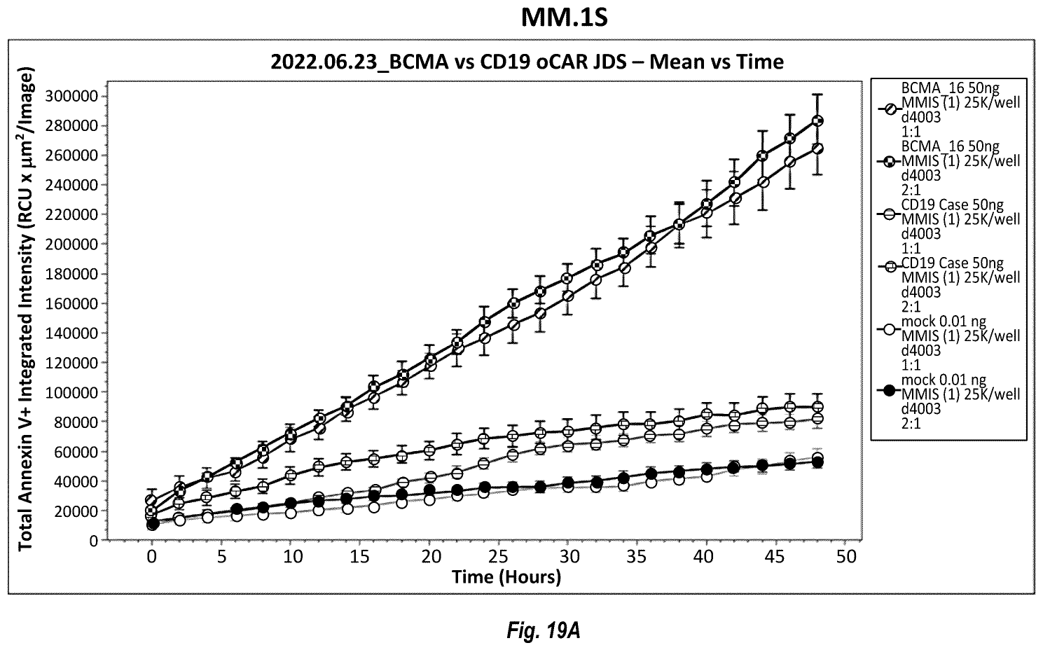

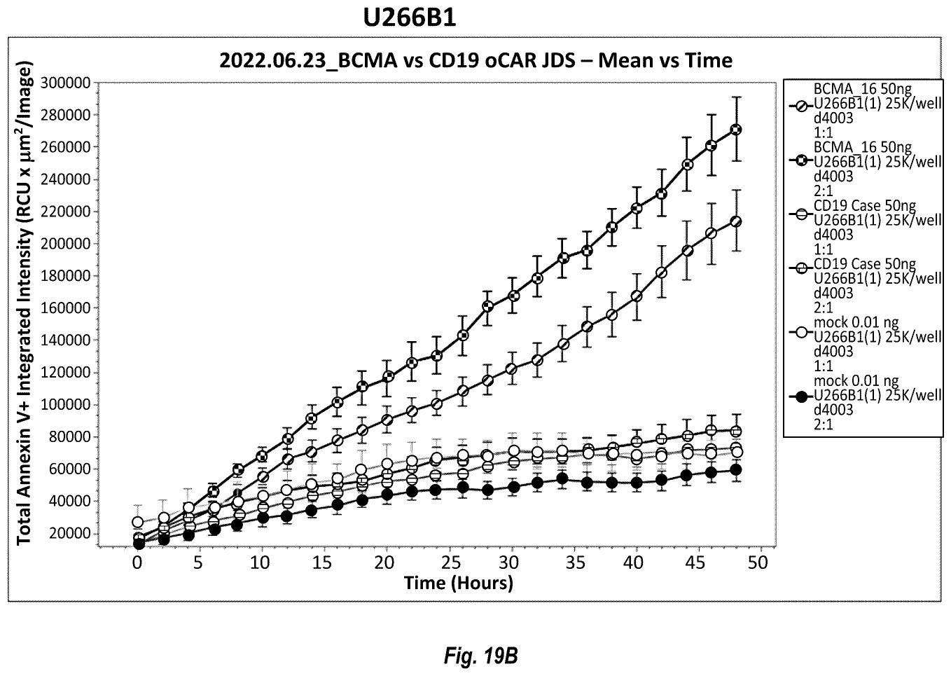

A HER2. BBC oCAR construct, HER2 280 oCAR construct, and a CD19 oCAR construct, were assessed against a base CD19 CAR control, and mock negative control for % target lysis using a FACS based cytotoxicity assay using an engineered HER2/K562 cell line (HER2-28ζ or HER2-BBζ), CD19/K562 cell line (CD19-28ζ or CD19-BBζ), and Nalm6 cell line (CD19+/HER2−). All oCAR constructs were electroporated into activated T cells to form CAR-T cells. The CAR-T cells were then co-cultured (2:1 E: T, 1:1 E: T, and 1:2 E: T) with target cells for 24 hours at 37° C. Target cells were engineered using the K562 cell line as a backbone (K562 cells overexpressing HER2 or CD19, GFP, and luciferase). 24 hours after co-culture, the CAR-T cells were evaluated using a single timepoint FACS assay to determine number of dead GFP positive target cells. See FIGS. 18A-18C. Both HER2 and CD19 CAR oCAR constructs were able to elicit target specific killing.

### Example 12: Circular RNA Constructs Comprising an Anti-BCMA Binder

Additional circular RNA constructs comprising an anti-BCMA binder were designed and assessed. See Table 8, below.

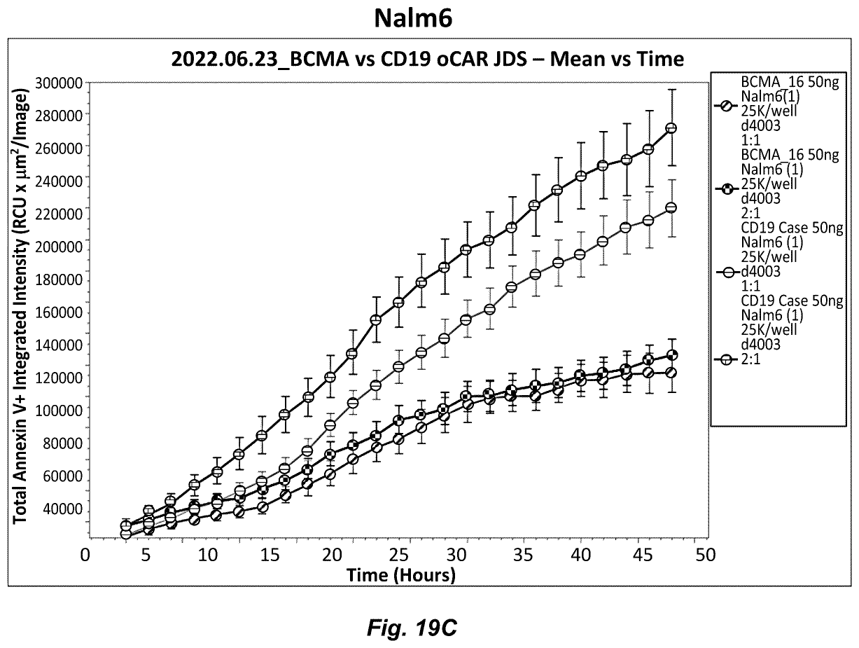

An oCAR construct comprising BCMA_16 was designed to comprise a combination of an IRES and expression sequences directed to surface expression of BCMA. Human T cells from Donor 4003 were activated with aCD3/aCD28. Two days post activation, the BCMA_16 oCAR construct, CD19 base construct, and mock negative control were electroporated via Nucleofect (50 ng oCAR/0.1e6 T cells). 24 hour later, the T cells were cocultured with MM. 1 s, U266B1 or Nalm 6 (CD19+/HER2−) cell lines analyzed at E: T=1:1 and 2:1 for target specific cytotoxicity for a span of three days via total accumulation of cell death. See FIGS. 19A-19C. BCMA_16 and CD19 (Base) oCARs showed target specific killing.

The IRES-CO sequences for the BCMA_16 construct is set forth in SEQ ID NO: 138:

(SEQ ID NO: 138)
GTGGCCACGCCCGGGCCACCGATACTTCCCTTCACTCCTTCGGGACTGTTGGGGA
GGAACACAACAGGGCTCCCCTGTTTTCCCATTCCTTCCCCCTTTTCCCAACCCCAA
CCGCCGTATCTGGTGGCGGCAAGACACACGGGTCTTTCCCTCTAAAGCACAATTG
TGTGTGTGTCCCAGGTCCTCCTGCGTACGGTGCGGGAGTGCTCCCACCCAACTGT
TGTAAGCCTGTCCAACGCGTCGTCCTGGCAAGACTATGACGTCGCATGTTCCGCT
GCGGATGCCGACCGGGTAACCGGTTCCCCAGTGTGTGTAGTGCGATCTTCCAGGT
CCTCCTGGTTGGCGTTGTCCAGAAACTGCTTCAGGTAAGTGGGGTGTGCCCAATC
CCTACAAAGGTTGATTCTTTCACCACCTTAGGAATGCTCCGGAGGTACCCCAGCA
ACAGCTGGGATCTGACCGGAGGCTAATTGTCTACGGGTGGTGTTTCCTTTTTCTTT
TCACACAACTCTACTGCTGACAACTCACTGACTATCCACTTGCTCTGTCACGATGG
CACTCCCGGTAACCGCCTTATTGCTTCCCCTTGCCCTCTTGCTCCACGCAGCACGC
CCCGATATAGTCTTGACTCAATCCCCACCCAGTTTGGCAATGTCATTAGGCAAAC
GAGCAACAATTTCATGTAGGGCATCCGAAAGTGTAACGATTTTGGGGAGTCATTT
AATTCATTGGTACCAACAAAAGCCTGGACAACCCCCGACGCTCTTGATCCAATTA
GCATCTAACGTCCAAACCGGAGTCCCCGCACGATTCTCAGGATCCGGTTCCCGGA
CTGATTTTACATTAACTATTGATCCGGTAGAGGAAGATGACGTCGCTGTCTATTAT
TGTCTTCAAAGTAGGACGATTCCACGGACATTCGGTGGCGGAACTAAATTGGAGA
TTAAAGGTTCCACCTCTGGTAGTGGGAAACCCGGGTCCGGTGAAGGGTCCACTAA
AGGCCAAATTCAACTCGTTCAATCCGGACCAGAACTGAAGAAGCCAGGAGAAAC
TGTCAAAATAAGCTGTAAAGCTTCCGGTTATACATTTACAGATTATTCCATAAATT
GGGTGAAAAGGGCGCCAGGAAAAGGGTTAAAGTGGATGGGTTGGATTAATACAG
AGACTCGGGAACCTGCATATGCTTATGATTTTAGGGGAAGGTTTGCCTTTTCTCTG
GAGACTTCCGCTTCAACTGCTTATCTCCAAATTAATAATCTTAAATATGAGGACA
CAGCAACATACTTCTGTGCTTTGGACTATAGTTATGCTATGGATTACTGGGGACA
AGGAACCAGTGTCACTGTAAGTTCCGCTGCTGCGACGACCACTCCTGCACCGCGA
CCACCCACTCCTGCCCCTACTATTGCTAGTCAACCACTTAGCTTGCGACCTGAGGC
ATGTCGGCCCGCGGCAGGTGGCGCAGTCCACACCAGGGGTTTAGACTTTGCTTGT
GATATTTATATTTGGGCACCACTCGCCGGGACTTGCGGTGTTCTTCTCTTGTCCCT
TGTTATAACTCTTTATTGTAAGCGCGGAAGGAAGAAATTGTTATATATTTTCAAA
CAACCTTTTATGCGACCCGTACAAACAACTCAGGAAGAGGACGGGTGTTCTTGTC
GGTTTCCAGAAGAGGAAGAGGGTGGGTGTGAACTCCGGGTCAAATTTAGTAGGT
CAGCAGATGCGCCGGCGTACCAACAAGGCCAAAACCAACTGTATAATGAACTCA
ATCTCGGTAGGCGTGAGGAATATGATGTCCTTGATAAAAGGCGCGGGAGAGATC
CAGAAATGGGCGGAAAACCACGGCGAAAGAATCCGCAGGAAGGGTTATATAAC
GAACTTCAAAAGGATAAAATGGCTGAAGCTTATTCCGAAATTGGCATGAAAGGA
GAGCGACGTAGGGGCAAAGGGCATGATGGCCTTTACCAAGGGCTCTCAACCGCT
ACAAAAGATACTTACGACGCTTTACATATGCAAGCACTTCCACCCAGG

TABLE 8
Additional Codon-IRES Constructs (BCMA)

| IRES/CO | IRES | Codon NT |
|---|---|---|
| Clone # | SEQ ID NO: | SEQ ID NO: |
| BCMA_1 | 16 | 103 |
| BCMA_2 | 17 | 103 |
| BCMA_3 | 8 | 103 |
| BCMA_4 | 18 | 103 |
| BCMA_5 | 8 | 104 |
| BCMA_6 | 8 | 105 |
| BCMA_7 | 8 | 106 |
| BCMA_8 | 8 | 107 |
| BCMA_9 | 8 | 108 |
| BCMA_10 | 8 | 109 |
| BCMA_11 | 8 | 110 |
| BCMA_12 | 8 | 111 |
| BCMA_13 | 8 | 112 |
| BCMA_14 | 8 | 113 |
| BCMA_15 | 8 | 114 |
| BCMA_16 | 17 | 115 |

### Example 13: In Vivo Dosing Post-Nalm6 Engraftment

The anti-CD19 oRNA-LNP construct described in Example 10 was assessed in vivo in mice engrafted with Nalm6 tumor cells and dosing schedules (every-other-day dosing post-engraftment, weekly dosing post-engraftment, and every-other-week dosing post-engraftment) were compared. Dose timing groups were evaluated at doses of 1.0 mg/kg, 0.3 mg/kg, and 0.1 mg/kg across four PBMC donors (two studies, two donors per study, A and B). The experimental protocol is shown in the table below.

NSG MHC I/II double knockout mice were engrafted with Nalm6-Luciferase (Luc) tumor cells and 4 days later were engrafted with human PBMCs. Starting on Day 5 post-Nalm6 engraftment, the mice were treated 4 times (as shown in the table below in the dosing days column) with the anti-CD19 oRNA-LNP, PBS control, or HER2 control as shown herein. Total flux (photons/second) was observed over time for the CD19 oRNA construct. Animals were then whole-body imaged twice weekly via IVIS to monitor luciferase expression from Nalm6 cells.

| | | | | | Dosing | | | |
|---|---|---|---|---|---|---|---|---|
| | | | | | Days | | | |
| | | Dose | # of | Dose | (Post- | LNP | Mice/ | PBMC |
| # | oRNA | (mg/kg) | Doses | Timing | Nalm6) | Formulation | Group | Donor |
| 1 | 1X | N/A | 4 | 1X/week | 5, 12, 19, | N/A | 5 | A |
| | PBS | | | | 26 | | | |
| 2 | HER2 | 1.0 | 4 | 1X/week | 5, 12, 19, | 1X TSS, −80 C. | 5 | A |
| | CAR | | | | 26 | | | |
| 3 | CD19 | 1.0 | 4 | 1X/week | 5, 12, 19, | IX TSS, −80 C. | 8 | A |
| | CAR | | | | 26 | | | |
| 4 | CD19 | 0.3 | 4 | 1X/week | 5, 12, 19, | 1X TSS, −80 C. | 8 | A |
| | CAR | | | | 26 | | | |
| 5 | CD19 | 0.1 | 4 | 1X/week | 5, 12, 19, | 1X TSS, −80 C. | 8 | A |
| | CAR | | | | 26 | | | |
| 6 | 1X | N/A | 4 | Every | 5, 19, 33, | N/A | 5 | A |
| | PBS | | | other | 47 | | | |
| | | | | week | | | | |
| 7 | HER2 | 1.0 | 4 | Every | 5, 19, 33, | 1X TSS, −80 C. | 5 | A |
| | CAR | | | other | 47 | | | |
| | | | | week | | | | |
| 8 | CD19 | 1.0 | 4 | Every | 5, 19, 33, | 1X TSS, −80 C. | 8 | A |
| | CAR | | | other | 47 | | | |
| | | | | week | | | | |
| 9 | CD19 | 0.3 | 4 | Every | 5, 19, 33, | 1X TSS, −80 C. | 8 | A |
| | CAR | | | other | 47 | | | |
| | | | | week | | | | |
| 10 | CD19 | 0.1 | 4 | Every | 5, 19, 33, | 1X TSS, −80 C. | 8 | A |
| | CAR | | | other | 47 | | | |
| | | | | week | | | | |
| 11 | 1X | N/A | 4 | 1X/week | 5, 12, 19, | N/A | 5 | B |
| | PBS | | | | 26 | | | |
| 12 | HER2 | 1.0 | 4 | 1X/week | 5, 12, 19, | 1X TSS, −80 C. | 5 | B |
| | CAR | | | | 26 | | | |
| 13 | CD19 | 1.0 | 4 | 1X/week | 5, 12, 19, | 1X TSS, −80 C. | 8 | B |
| | CAR | | | | 26 | | | |
| 14 | CD19 | 0.3 | 4 | 1X/week | 5, 12, 19, | 1X TSS, −80 C. | 8 | B |
| | CAR | | | | 26 | | | |
| 15 | CD19 | 0.1 | 4 | 1X/week | 5, 12, 19, | 1X TSS, −80 C. | 8 | B |
| | CAR | | | | 26 | | | |
| 16 | 1X | N/A | 4 | Every | 5, 19, 33, | N/A | 5 | B |
| | PBS | | | other | 47 | | | |
| | | | | week | | | | |
| 17 | HER2 | 1.0 | 4 | Every | 5, 19, 33, | 1X TSS, −80 C. | 5 | B |
| | CAR | | | other | 47 | | | |
| | | | | week | | | | |
| 18 | CD19 | 1.0 | 4 | Every | 5, 19, 33, | 1X TSS, −80 C. | 8 | B |
| | CAR | | | other | 47 | | | |
| | | | | week | | | | |
| 19 | CD19 | 0.3 | 4 | Every | 5, 19, 33, | 1X TSS, −80 C. | 8 | B |
| | CAR | | | other | 47 | | | |
| | | | | week | | | | |
| 20 | CD19 | 0.1 | 4 | Every | 5, 19, 33, | 1X TSS, −80 C. | 8 | B |
| | CAR | | | other | 47 | | | |
| | | | | week | | | | |

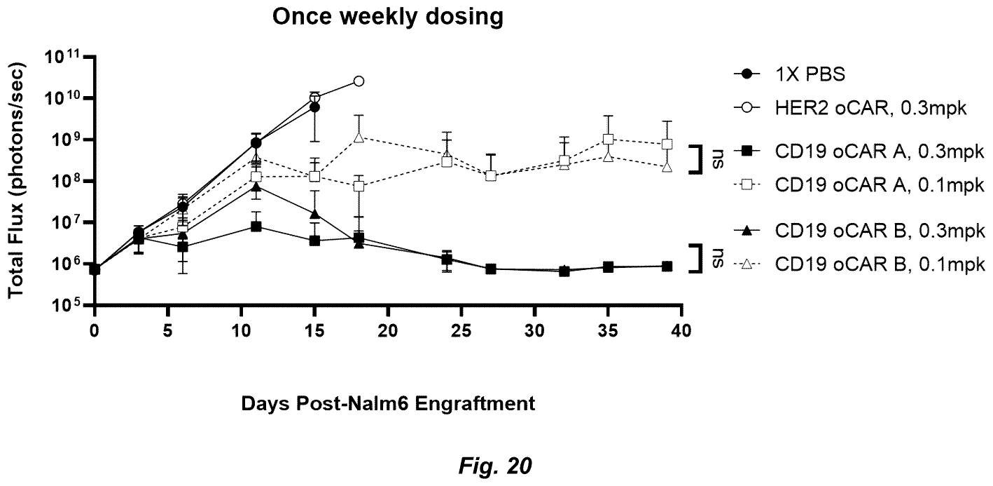

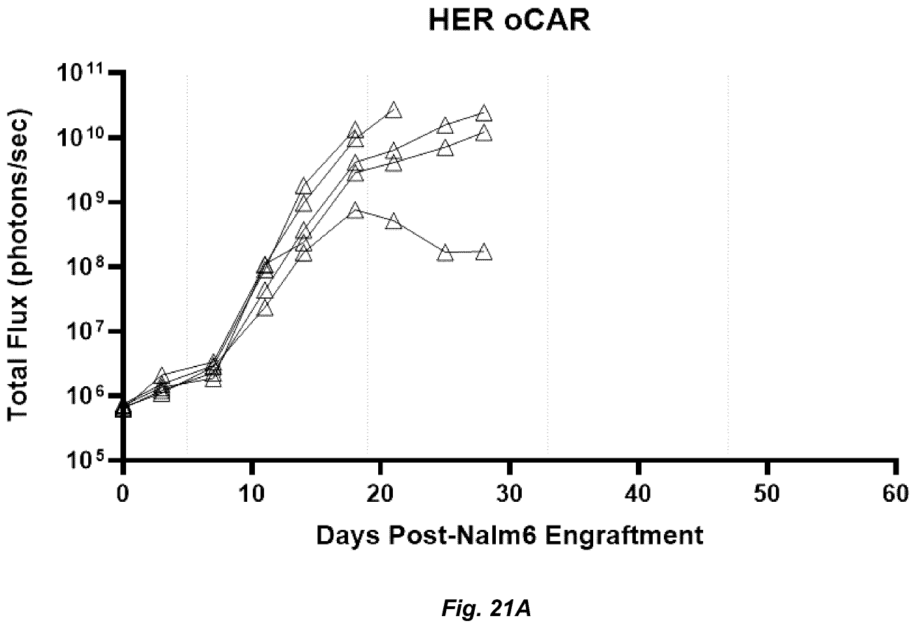

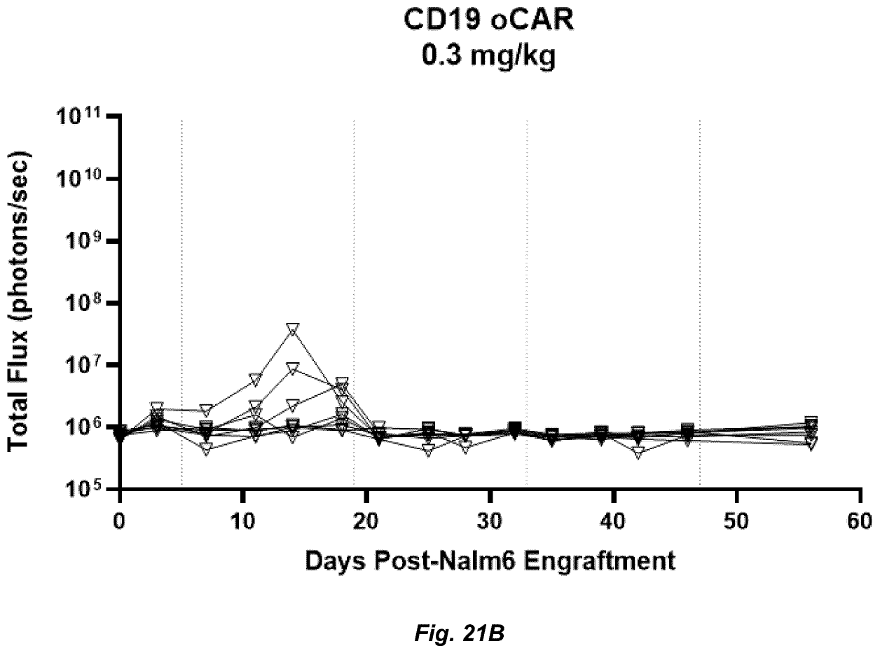

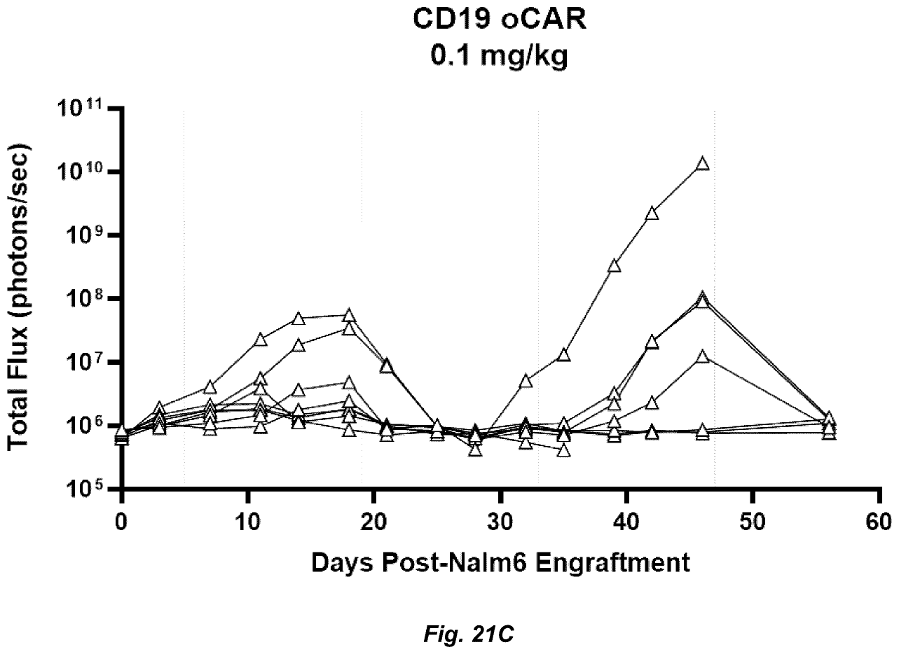

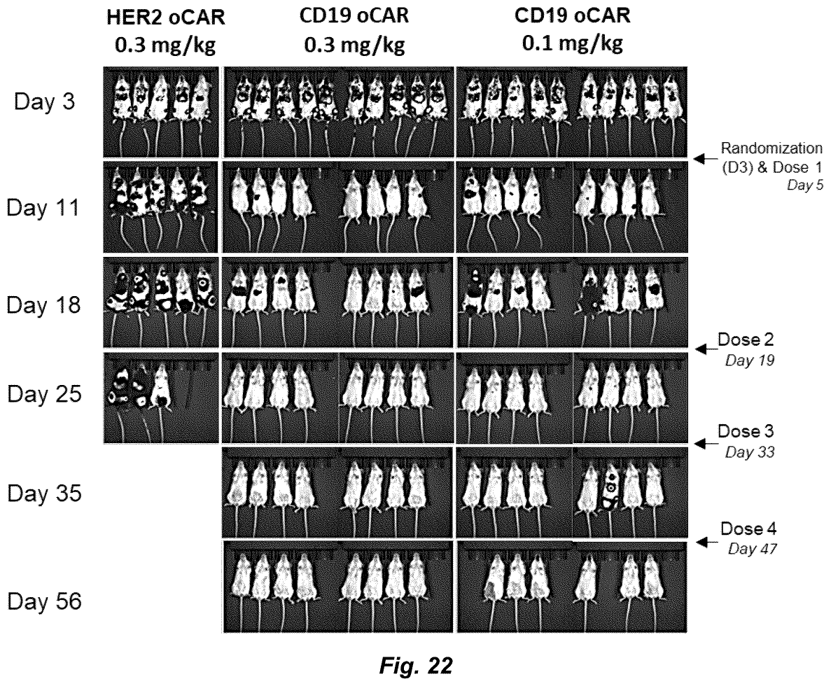

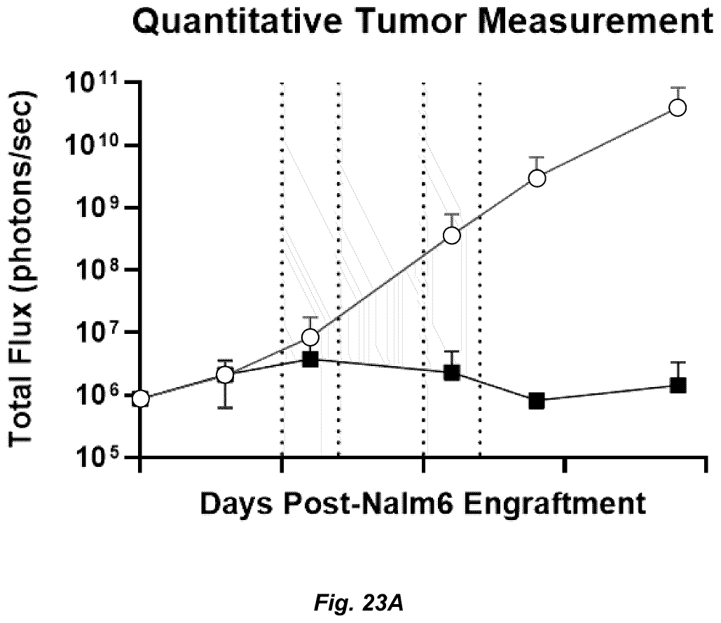

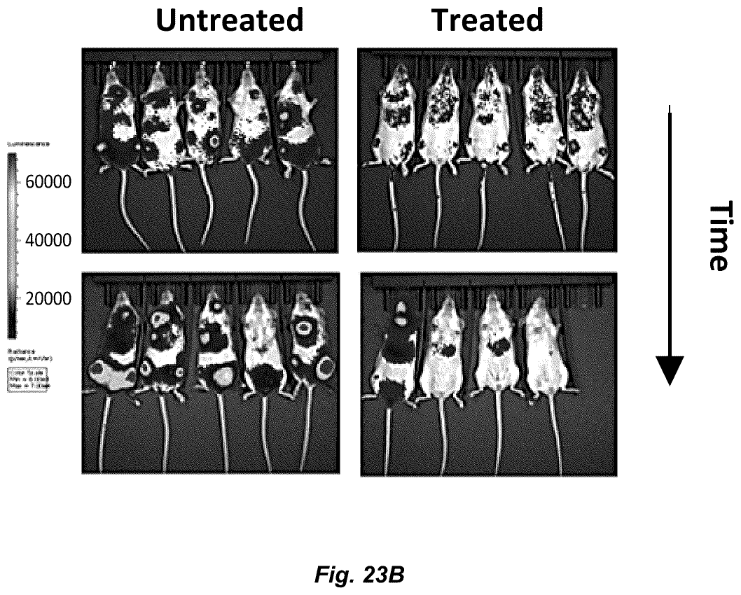

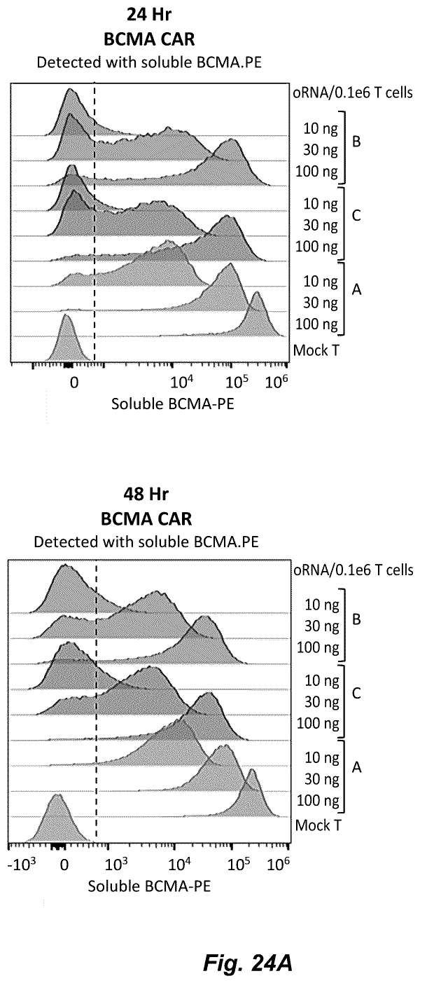

The CD19 oRNA-LNP treated animals showed tumor control and improved survival compared to controls treated with the HER2 oRNA construct described herein. Tumor control for weekly dosing is shown in FIG. 20. Tumor control for every-other-week dosing is shown in FIGS. 21A-C and FIG. 22. Tumor control was observed for up to 56 days in animals treated with every-other-week dosing. FIG. 23A and FIG. 23B show tumor control for CD19 oRNA-LNP as compared to a control.

### Example 14: Expression of Circular RNAs Encoding Anti-BCMA CARS

### Expression of Circular RNAs Encoding Anti-BCMA CAR In Vitro

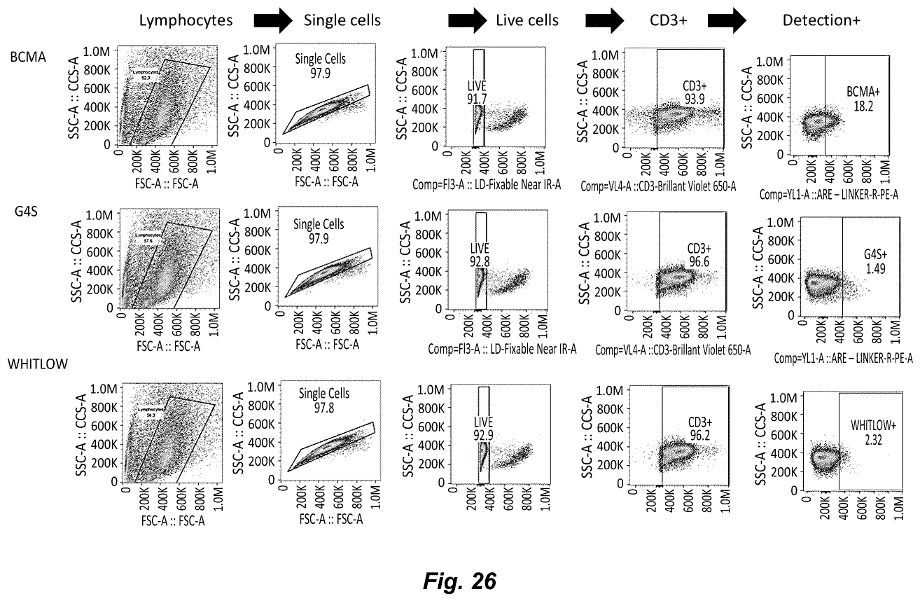

T cells from a single donor were activated for 3 days with anti-CD3 and CD28 beads and allowed to rest for 24 hours. Concurrently, engineered circular RNA constructs were designed to encode a BCMA-41BBζ chimeric antigen receptor (CAR). For comparison purposes, “mock” or control circular RNAs encoded a HER2 or CD19 CAR. The circular RNAs were developed from an in vitro translation (IVT) reaction of DNA comprising a T7 polymerase promoter, permuted Anabaena intron exon segments, internal ribosome entry site (IRES) from Caprine Kobuvirus, Hunnivirus, Apodemus Picornavirus, or Picornavirales internal spacers, optionally internal homology arms, and a Xlab restriction site. DNA templates comprised of sequences from the table below. The donor T cells were electroporated with the circular RNAs at either 10 ng, 30 ng, or 100 ng per 0.1×10⁶ T cell to form CAR-T cells. As a control, “Mock” T cells not electroporated with circular RNAs were analyzed. T cells were then allowed to rest for 24 hours after electroporation. 24 hours after electroporation, the CAR-T cells were counted and assessed for BCMA CAR expression. Resulting circular RNA with anti-BCMA CAR encoding regions were given a commercially available soluble BCMA detection reagent containing R-phycoerythrin (PE) fluorophore (e.g., from AcroBiosystems, Delaware), anti-Whitlow PE linker antibody detection reagent, or an anti-G4S linker PE detection reagent (e.g., G4S-AF647). Anti-BCMA, CD19, or HER2 expression was assessed using fluorescence activated cell sorting (FACS) and gating flow cytometry methods known in the art (e.g., illustrated in FIG. 26 for T cells electroporated with circular RNAs derived from DNA Template B and dosed at 10 ng per 0.1×10⁶ T cells) at one or more timepoints from 24 to 72 hours post electroporation.

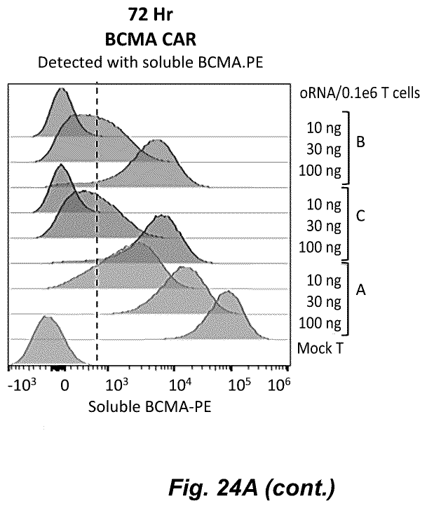

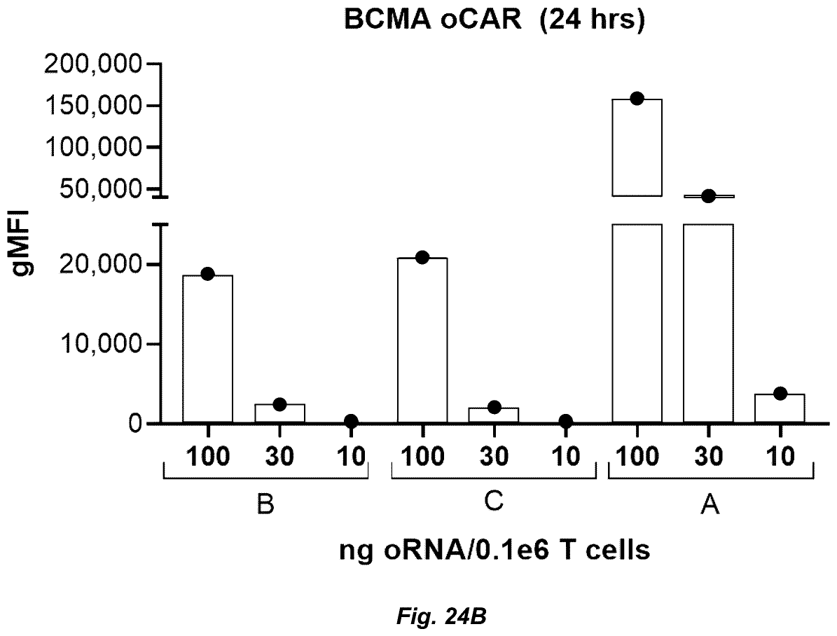

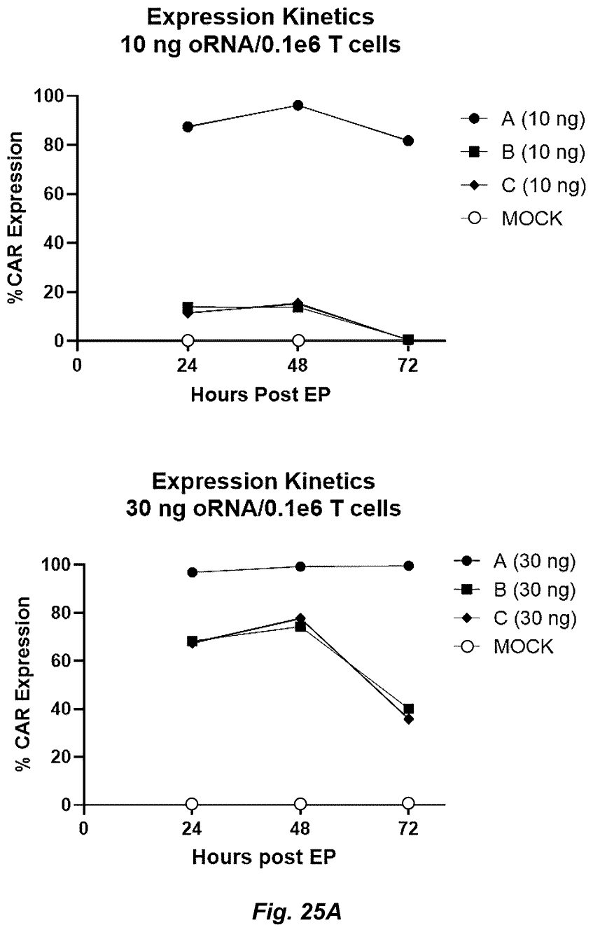

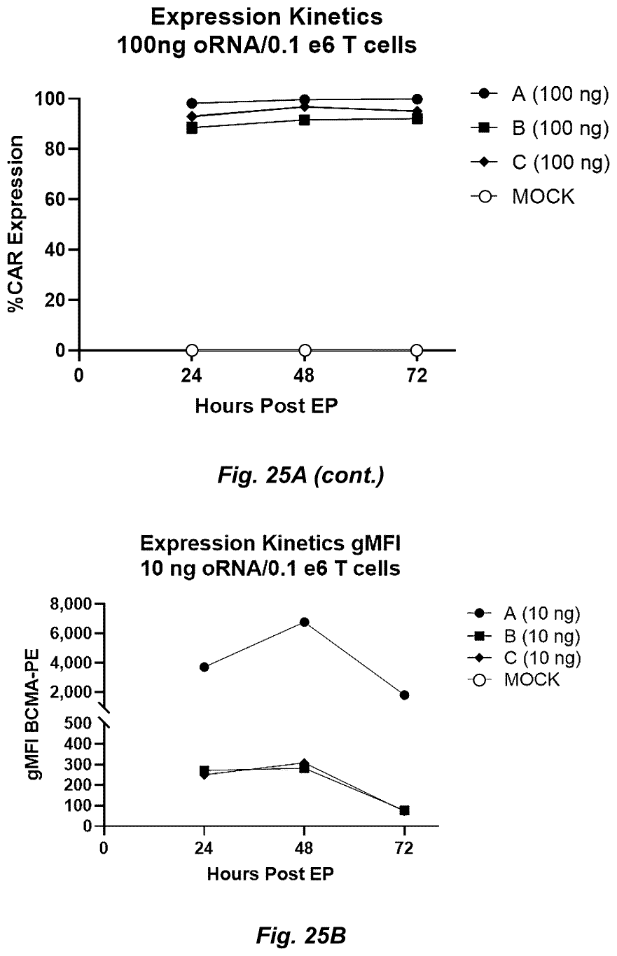

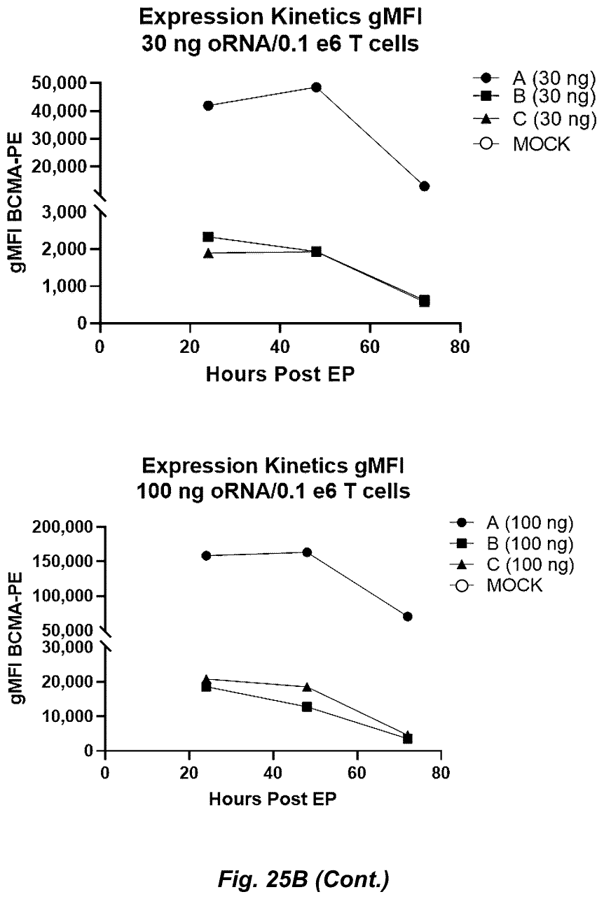

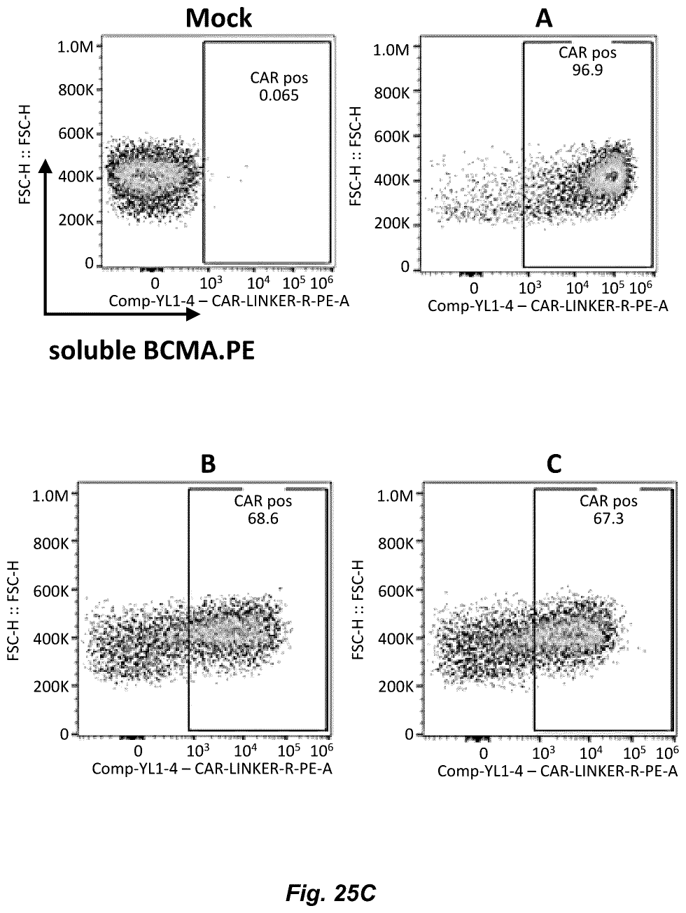

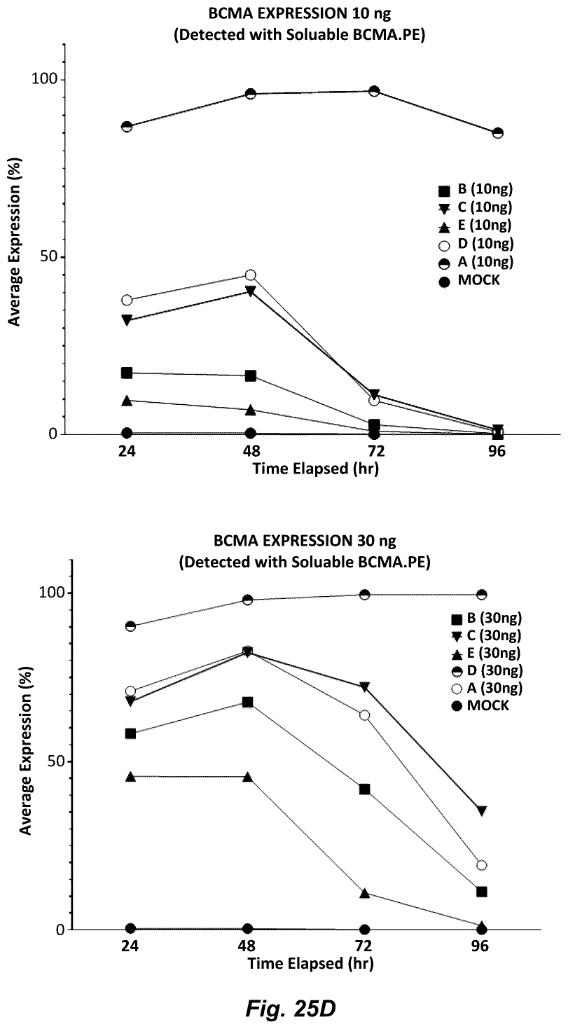

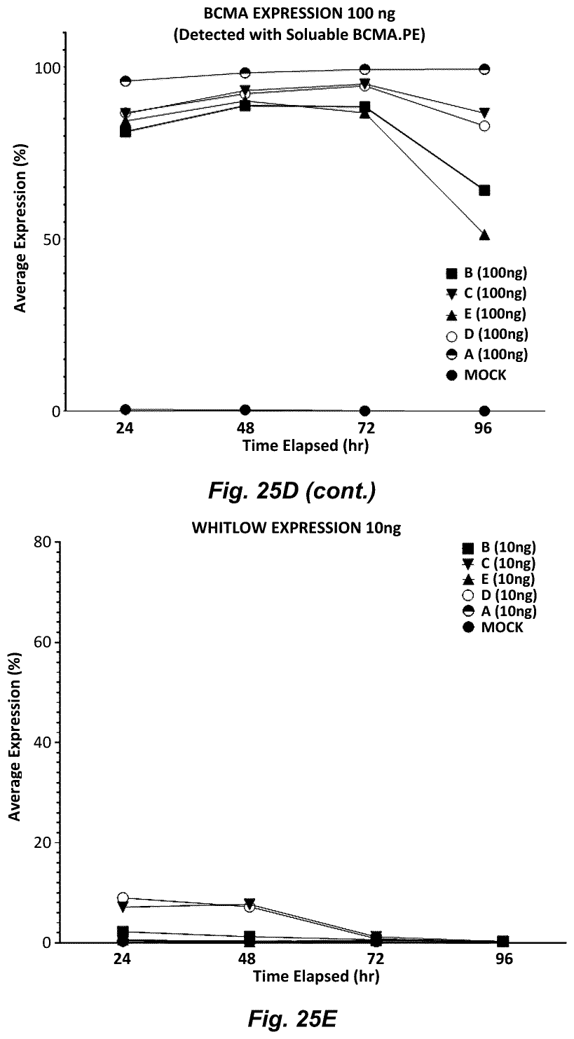

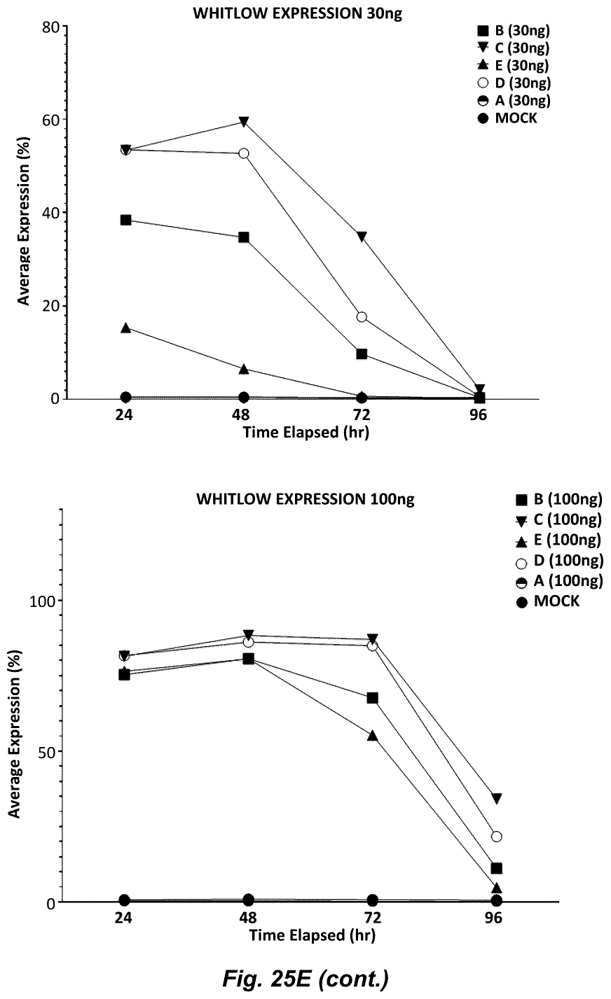

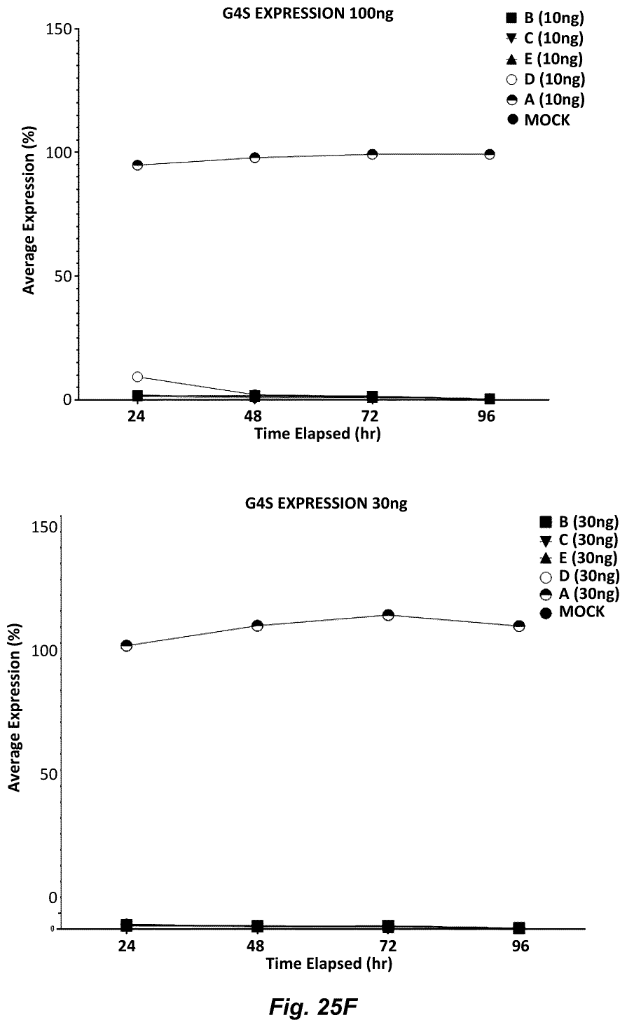

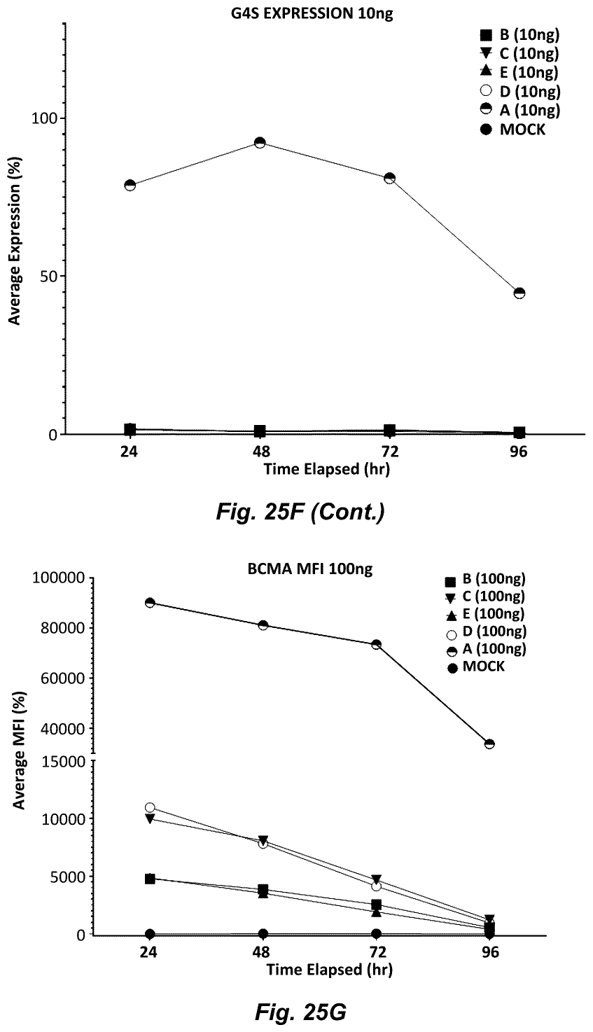

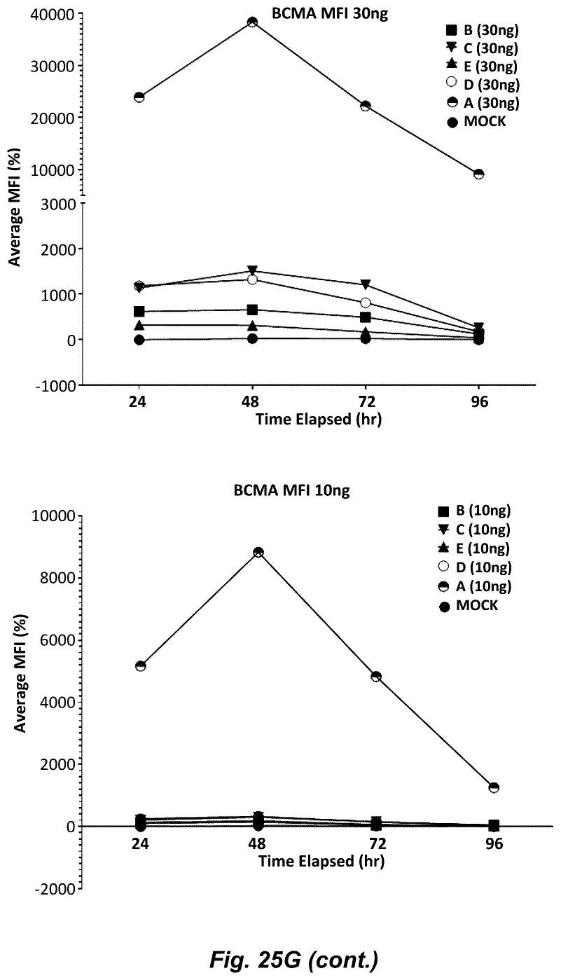

FIG. 24A provides the flow cytometry histogram, for each of the circular RNA constructs at the three different dosages collected at 24 hours, 48 hours and 72 hours. FIG. 24B shows the gMFI expression of each of the circular RNA constructs at the three different dosages collected at 24 hours. FIGS. 25A, 25D-25F depict percent CAR expression detected by either soluble BCMA PE, anti-Whitlow PE or anti-G4S linker PE detection reagent. FIGS. 25B and 25G gMFI and average MFI % respectively over the span of 24 hours to 96 hours post electroporation for each of the three dosages. FIG. 25C illustrates FACS imaging of the BCMA CARs as detected by the soluble BCMA detection reagent at 24 hours post electroporation.

TABLE α1

| | Internal Ribosome Entry | | CAR |
|---|---|---|---|
| ID | Site (IRES) Sequence | CAR Sequence | Sequence |
| DNA | CCCCCCTCCCCCCCTTCCCT | ATGGCTCTCCCCGTGACCGCTCTGCT | MALPVTALLL |
| Template A | TCCCTTTGCAACGCAACAAT | GCTCCCTCTGGCCCTCCTTCTGCACG | PLALLLHAAR |
| (Construct | TGTAAGTGCCCTCACCTGTC | CAGCCAGACCACAGGTCAAGCTGGAG | PQVKLEESGG |
| A) | AATTGGGACCACCACTTTCA | GAGTCTGGTGGCGGTCTGGTGCAGGC | GLVQAGRSLR |
| | GTGACCCCATGCGAAGTGCT | AGGGAGGAGCCTGAGGCTGAGCTGTG | LSCAASEHTE |
| | GAGAGAAAGGAAGCTTTCTT | CAGCTTCCGAGCACACATTCTCAAGC | SSHVMGWFRQ |
| | ACCCTTCATTTGTGAACCCA | CACGTCATGGGGTGGTTCAGACAGGC | APGKERESVA |
| | CTGGTCTAAGCCGCTTGGAA | TCCCGGTAAAGAGAGGGAGTCCGTCG | VIGWRDISTS |
| | TACGATGAGTGGAAAAGTTC | CCGTGATCGGATGGCGGGACATCTCC | YADSVKGRFT |
| | ATTCTTAATGGAGTGAAACA | ACCTCCTACGCCGACTCTGTGAAGGG | ISRDNAKKTL |
| | TGCTTAAATTTCCAGCTCGT | CCGGTTCACAATCTCACGCGATAATG | YLQMNSLKPE |
| | GCTGGTCTTTCCAGTACGGG | CCAAGAAGACACTGTATCTGCAGATG | DTAVYYCAAR |
| | GCGGCCCTGTCTGGCCGTAA | AATTCCTTGAAGCCCGAAGACACCGC | RIDAADEDSW |
| | TTCTTCAGAGTGTCACGCCA | CGTCTATTACTGTGCTGCTAGACGGA | GQGTQVTVSS |
| | CACTTGTGGATCTCACGTGC | TCGACGCTGCCGACTTCGACAGCTGG | GGGGSGGGGS |
| | CACATGACAGCGCTACAGCT | GGACAGGGTACCCAAGTGACCGTTTC | GGGGSEVQLV |
| | GGAACTGGGTGCTTGGTGCC | CTCCGGAGGCGGAGGTTCTGGAGGAG | ESGGGLVQAG |
| | CATGGAGTAACAGCGAAAAG | GTGGGTCAGGTGGAGGTGGCTCCGAG | GSLRLSCAAS |
| | TGTTAGATCAAGCCTTGCTT | GTGCAGCTGGTCGAGTCTGGCGGTGG | GRTFTMGWER |
| | GGGCTATGAGCCTGCGGAAC | CTTGGTCCAGGCTGGAGGCAGTCTCA | QAPGKEREFV |
| | AACAACTGGTAACAGTTGCC | GACTCTCCTGCGCTGCTTCAGGGCGG | AAISLSPTLA |
| | TCAGGGGCCGAAAGCCACGG | ACCTTCACCATGGGCTGGTTCAGGCA | YYAESVKGRE |
| | TGTTAACAGCACCCTCATAG | GGCCCCAGGTAAGGAGAGGGAGTTCG | TISRDNAKNT |
| | TTTGATCCACCTCAGGGTGG | TGGCCGCCATCTCCCTCTCCCCTACC | VVLQMNSLKP |
| | TGATGTTTAGCAGTTAGTAG | CTGGCATACTACGCTGAGTCCGTGAA | EDTALYYCAA |
| | TTGCCAATCTGTGTTCACTG | GGGACGGTTTACCATCTCCCGGGATA | DRKSVMSIRP |
| | AAATCTCGGCATACCGTGTA | ACGCAAAGAACACTGTGGTCCTCCAA | DYWGQGTQVT |
| | GTGTACAGGGGTGAAGGATG | ATGAACTCCCTCAAACCCGAGGACAC | VSSTSTTTPA |
| | CCCAGAAGGTACCCGTAGGT | CGCTCTCTACTATTGTGCCGCAGATC | PRPPTPAPTI |
| | AACCTTAAGAGACTATGGAT | GGAAGAGCGTCATGTCCATCCGGCCC | ASQPLSLRPE |
| | CTGATCTGGGGCCTTGTCCG | GATTACTGGGGCCAAGGCACACAGGT | ACRPAAGGAV |
| | GAGTGCTTTACACACGGCTC | GACTGTGTCCAGCACCTCCACCACCA | HTRGLDFACD |
| | AAGGTTAAAAAACGTCTAGC | CCCCAGCACCAAGGCCTCCAACCCCT | IYIWAPLAGT |
| | CCCACAGAGCCCGAGGGATT | GCACCAACCATCGCCTCCCAGCCACT | CGVLLLSLVI |
| | CGGGTTTTCCCTTTAAAAAC | GTCTTTGCGGCCAGAAGCATGCCGCC | TLYCKRGRKK |
| | CCGACTAGAGCTTATGGTGA | CAGCAGCAGGTGGAGCCGTGCATACA | LLYIFKQPFM |
| | CAATTATTGCTGTTCAGACG | AGAGGCCTGGACTTCGCCTGCGATAT | RPVQTTQEED |
| | AACAGTGTAATTGTTGTCTA | CTACATCTGGGCTCCTCTGGCCGGAA | GCSCRFPEEE |
| | TTCACAGCAGTTCTATCAGA | CATGCGGAGTCCTGCTCTTGTCCCTG | EGGCELRVKF |
| | GCTTTTCCCACAACGGATCT | GTGATCACCCTGTACTGCAAGCGGGG | SRSADAPAYQ |
| | TCTTGGCAAGCAAATACAGC | TCGGAAGAAGCTCCTCTACATCTTCA | QGQNQLYNEL |
| | AGGAGTCAAT (SEQ ID | AGCAGCCCTTCATGAGACCCGTCCAG | NLGRREEYDV |
| | NO: 8) | ACCACCCAGGAGGAGGACGGGTGCTC | LDKRRGRDPE |
| | | ATGCAGGTTCCCCGAAGAGGAGGAGG | MGGKPRRKNP |
| | | GTGGCTGTGAGCTGCGGGTGAAGTTC | QEGLYNELQK |
| | | AGCAGGTCAGCAGACGCCCCTGCCTA | DKMAEAYSEI |
| | | TCAGCAGGGCCAAAACCAGTIGTACA | GMKGERRRGK |
| | | ACGAGCTGAATCTGGGGAGACGGGAG | GHDGLYQGLS |
| | | GAGTACGATGTCCTTGACAAGAGAAG | TATKDTYDAL |
| | | GGGCCGGGATCCAGAGATGGGCGGGA | HMQALPPR |
| | | AGCCAAGACGGAAGAATCCTCAGGAG | (SEQ ID NO: |
| | | GGTCTGTATAACGAGCTGCAGAAGGA | 122) |
| | | CAAGATGGCCGAGGCCTACTCCGAGA | |
| | | TCGGCATGAAAGGGGAGCGCCGCAGA | |
| | | GGAAAAGGTCACGATGGTCTGTACCA | |
| | | GGGGTTGAGCACCGCTACCAAGGATA | |
| | | CTTACGACGCTCTGCACATGCAAGCT | |
| | | CTGCCACCCCGG (SEQ ID NO: | |
| | | 106) | |
| DNA | GTGGCCACGCCCGGGCCACC | ATGGCACTCCCGGTAACCGCCTTATT | MALPVTALLL |
| Template B | GATACTTCCCTTCACTCCTT | GCTTCCCCTTGCCCTCTTGCTCCACG | PLALLLHAAR |
| (Construct | CGGGACTGTTGGGGAGGAAC | CAGCACGCCCCGATATAGTCTTGACT | PDIVLTQSPP |
| B) | ACAACAGGGCTCCCCTGTTT | CAATCCCCACCCAGTTTGGCAATGTC | SLAMSLGKRA |
| | TCCCATTCCTTCCCCCTTTT | ATTAGGCAAACGAGCAACAATTTCAT | TISCRASESV |
| | CCCAACCCCAACCGCCGTAT | GTAGGGCATCCGAAAGTGTAACGATT | TILGSHLIHW |
| | CTGGTGGCGGCAAGACACAC | TTGGGGAGTCATTTAATTCATTGGTA | YQQKPGQPPT |
| | GGGTCTTTCCCTCTAAAGCA | CCAACAAAAGCCTGGACAACCCCCGA | LLIQLASNVQ |
| | CAATTGTGTGTGTGTCCCAG | CGCTCTTGATCCAATTAGCATCTAAC | TGVPARFSGS |
| | GTCCTCCTGCGTACGGTGCG | GTCCAAACCGGAGTCCCCGCACGATT | GSRTDFTLTI |
| | GGAGTGCTCCCACCCAACTG | CTCAGGATCCGGTTCCCGGACTGATT | DPVEEDDVAV |
| | TTGTAAGCCTGTCCAACGCG | TTACATTAACTATTGATCCGGTAGAG | YYCLQSRTIP |
| | TCGTCCTGGCAAGACTATGA | GAAGATGACGTCGCTGTCTATTATTG | RTFGGGTKLE |
| | CGTCGCATGTTCCGCTGCGG | TCTTCAAAGTAGGACGATTCCACGGA | IKGSTSGSGK |
| | ATGCCGACCGGGTAACCGGT | CATTCGGTGGCGGAACTAAATTGGAG | PGSGEGSTKG |
| | TCCCCAGTGTGTGTAGTGCG | ATTAAAGGTTCCACCTCTGGTAGTGG | QIQLVQSGPE |
| | ATCTTCCAGGTCCTCCTGGT | GAAACCCGGGTCCGGTGAAGGGTCCA | LKKPGETVKI |
| | TGGCGTTGTCCAGAAACTGC | CTAAAGGCCAAATTCAACTCGTTCAA | SCKASGYTFT |
| | TTCAGGTAAGTGGGGTGTGC | TCCGGACCAGAACTGAAGAAGCCAGG | DYSINWVKRA |
| | CCAATCCCTACAAAGGTTGA | AGAAACTGTCAAAATAAGCTGTAAAG | PGKGLKWMGW |
| | TTCTTTCACCACCTTAGGAA | CTTCCGGTTATACATTTACAGATTAT | INTETREPAY |
| | TGCTCCGGAGGTACCCCAGC | TCCATAAATTGGGTGAAAAGGGCGCC | AYDERGRFAF |
| | AACAGCTGGGATCTGACCGG | AGGAAAAGGGTTAAAGTGGATGGGTT | SLETSASTAY |
| | AGGCTAATTGTCTACGGGTG | GGATTAATACAGAGACTCGGGAACCT | LQINNLKYED |
| | GTGTTTCCTTTTTCTTTTCA | GCATATGCTTATGATTTTAGGGGAAG | TATYFCALDY |
| | CACAACTCTACTGCTGACAA | GTTTGCCTTTTCTCTGGAGACTTCCG | SYAMDYWGQG |
| | CTCACTGACTATCCACTTGC | CTTCAACTGCTTATCTCCAAATTAAT | TSVTVSSAAA |
| | TCTGTCACG (SEQ ID NO: | AATCTTAAATATGAGGACACAGCAAC | TTTPAPRPPT |
| | 17; | ATACTTCTGTGCTTTGGACTATAGTT | PAPTIASQPL |
| | | ATGCTATGGATTACTGGGGACAAGGA | SLRPEACRPA |
| | | ACCAGTGTCACTGTAAGTTCCGCTGC | AGGAVHTRGL |
| | | TGCGACGACCACTCCTGCACCGCGAC | DFACDIYIWA |
| | | CACCCACTCCTGCCCCTACTATTGCT | PLAGTCGVLL |
| | | AGTCAACCACTTAGCTTGCGACCTGA | LSLVITLYCK |
| | | GGCATGTCGGCCCGCGGCAGGTGGCG | RGRKKLLYIF |
| | | CAGTCCACACCAGGGGTTTAGACTTT | KQPFMRPVQT |
| | | GCTTGTGATATTTATATTTGGGCACC | TQEEDGCSCR |
| | | ACTCGCCGGGACTTGCGGTGTTCTTC | FPEEEEGGCE |
| | | TCTTGTCCCTTGTTATAACTCTTTAT | LRVKFSRSAD |
| | | TGTAAGCGCGGAAGGAAGAAATTGTT | APAYQQGQNQ |
| | | ATATATTTTCAAACAACCTTTTATGC | LYNELNLGRR |
| | | GACCCGTACAAACAACTCAGGAAGAG | EEYDVLDKRR |
| | | GACGGGTGTTCTTGTCGGTTTCCAGA | GRDPEMGGKP |
| | | AGAGGAAGAGGGTGGGTGTGAACTCC | RRKNPQEGLY |
| | | GGGTCAAATTTAGTAGGTCAGCAGAT | NELQKDKMAE |
| | | GCGCCGGCGTACCAACAAGGCCAAAA | AYSEIGMKGE |
| | | CCAACTGTATAATGAACTCAATCTCG | RRRGKGHDGL |
| | | GTAGGCGTGAGGAATATGATGTCCTT | YQGLSTATKD |
| | | GATAAAAGGCGCGGGAGAGATCCAGA | TYDALHMQAL |
| | | AATGGGCGGAAAACCACGGCGAAAGA | PPR (SEQ ID |
| | | ATCCGCAGGAAGGGTTATATAACGAA | NO: 119) |
| | | CTTCAAAAGGATAAAATGGCTGAAGC | |
| | | TTATTCCGAAATTGGCATGAAAGGAG | |
| | | AGCGACGTAGGGGCAAAGGGCATGAT | |
| | | GGCCTTTACCAAGGGCTCTCAACCGC | |
| | | TACAAAAGATACTTACGACGCTTTAC | |
| | | ATATGCAAGCACTTCCACCCAGG | |
| | | (SEQ ID NO: 103) | |
| DNA | CCCCCCTCCCCCCCTTCCCT | ATGGCACTCCCGGTAACCGCCTTATT | MALPVTALLL |
| Template C | TCCCTTTGCAACGCAACAAT | GCTTCCCCTTGCCCTCTTGCTCCACG | PLALLLHAAR |
| (Construct | TGTAAGTGCCCTCACCTGTC | CAGCACGCCCCGATATAGTCTTGACT | PDIVLTQSPP |
| C) | AATTGGGACCACCACTTTCA | CAATCCCCACCCAGTTTGGCAATGTC | SLAMSLGKRA |
| | GTGACCCCATGCGAAGTGCT | ATTAGGCAAACGAGCAACAATTTCAT | TISCRASESV |
| | GAGAGAAAGGAAGCTTTCTT | GTAGGGCATCCGAAAGTGTAACGATT | TILGSHLIHW |
| | ACCCTTCATTTGTGAACCCA | TTGGGGAGTCATTTAATTCATTGGTA | YQQKPGQPPT |
| | CTGGTCTAAGCCGCTTGGAA | CCAACAAAAGCCTGGACAACCCCCGA | LLIQLASNVQ |
| | TACGATGAGTGGAAAAGTTC | CGCTCTTGATCCAATTAGCATCTAAC | TGVPARFSGS |
| | ATTCTTAATGGAGTGAAACA | GTCCAAACCGGAGTCCCCGCACGATT | GSRTDFTLTI |
| | TGCTTAAATTTCCAGCTCGT | CTCAGGATCCGGTTCCCGGACTGATT | DPVEEDDVAV |
| | GCTGGTCTTTCCAGTACGGG | TTACATTAACTATTGATCCGGTAGAG | YYCLQSRTIP |
| | GCGGCCCTGTCTGGCCGTAA | GAAGATGACGTCGCTGTCTATTATTG | RTFGGGTKLE |
| | TTCTTCAGAGTGTCACGCCA | TCTTCAAAGTAGGACGATTCCACGGA | IKGSTSGSGK |
| | CACTTGTGGATCTCACGTGC | CATTCGGTGGCGGAACTAAATTGGAG | PGSGEGSTKG |
| | CACATGACAGCGCTACAGCT | ATTAAAGGTTCCACCTCTGGTAGTGG | QIQLVQSGPE |
| | GGAACTGGGTGCTTGGTGCC | GAAACCCGGGTCCGGTGAAGGGTCCA | LKKPGETVKI |
| | CATGGAGTAACAGCGAAAAG | CTAAAGGCCAAATTCAACTCGTTCAA | SCKASGYTFT |
| | TGTTAGATCAAGCCTTGCTT | TCCGGACCAGAACTGAAGAAGCCAGG | DYSINWVKRA |
| | GGGCTATGAGCCTGCGGAAC | AGAAACTGTCAAAATAAGCTGTAAAG | PGKGLKWMGW |
| | AACAACTGGTAACAGTTGCC | CTTCCGGTTATACATTTACAGATTAT | INTETREPAY |
| | TCAGGGGCCGAAAGCCACGG | TCCATAAATTGGGTGAAAAGGGCGCC | AYDERGRFAF |
| | TGTTAACAGCACCCTCATAG | AGGAAAAGGGTTAAAGTGGATGGGTT | SLETSASTAY |
| | TTTGATCCACCTCAGGGTGG | GGATTAATACAGAGACTCGGGAACCT | LQINNLKYED |
| | TGATGTTTAGCAGTTAGTAG | GCATATGCTTATGATTTTAGGGGAAG | TATYFCALDY |
| | TTGCCAATCTGTGTTCACTG | GTTTGCCTTTTCTCTGGAGACTTCCG | SYAMDYWGQG |
| | AAATCTCGGCATACCGTGTA | CTTCAACTGCTTATCTCCAAATTAAT | TSVTVSSAAA |
| | GTGTACAGGGGTGAAGGATG | AATCTTAAATATGAGGACACAGCAAC | TTTPAPRPPT |
| | CCCAGAAGGTACCCGTAGGT | ATACTTCTGTGCTTTGGACTATAGTT | PAPTIASQPL |
| | AACCTTAAGAGACTATGGAT | ATGCTATGGATTACTGGGGACAAGGA | SLRPEACRPA |
| | CTGATCTGGGGCCTTGTCCG | ACCAGTGTCACTGTAAGTTCCGCTGC | AGGAVHTRGL |
| | GAGTGCTTTACACACGGCTC | TGCGACGACCACTCCTGCACCGCGAC | DFACDIYIWA |
| | AAGGTTAAAAAACGTCTAGC | CACCCACTCCTGCCCCTACTATTGCT | PLAGTCGVLL |
| | CCCACAGAGCCCGAGGGATT | AGTCAACCACTTAGCTTGCGACCTGA | LSLVITLYCK |
| | CGGGTTTTCCCTTTAAAAAC | GGCATGTCGGCCCGCGGCAGGTGGCG | RGRKKLLYIF |
| | CCGACTAGAGCTTATGGTGA | CAGTCCACACCAGGGGTTTAGACTTT | KQPFMRPVQT |
| | CAATTATTGCTGTTCAGACG | GCTTGTGATATTTATATTTGGGCACC | TQEEDGCSCR |
| | AACAGTGTAATTGTTGTCTA | ACTCGCCGGGACTTGCGGTGTTCTTC | FPEEEEGGCE |
| | TTCACAGCAGTTCTATCAGA | TCTTGTCCCTTGTTATAACTCTTTAT | LRVKFSRSAD |
| | GCTTTTCCCACAACGGATCT | TGTAAGCGCGGAAGGAAGAAATTGTT | APAYQQGQNQ |
| | TCTTGGCAAGCAAATACAGC | ATATATTTTCAAACAACCTTTTATGC | LYNELNLGRR |
| | AGGAGTCAAT (SEQ ID | GACCCGTACAAACAACTCAGGAAGAG | EEYDVLDKRR |
| | NO: 8) | GACGGGTGTTCTTGTCGGTTTCCAGA | GRDPEMGGKP |
| | | AGAGGAAGAGGGTGGGTGTGAACTCC | RRKNPQEGLY |
| | | GGGTCAAATTTAGTAGGTCAGCAGAT | NELQKDKMAE |
| | | GCGCCGGCGTACCAACAAGGCCAAAA | AYSEIGMKGE |
| | | CCAACTGTATAATGAACTCAATCTCG | RRRGKGHDGL |
| | | GTAGGCGTGAGGAATATGATGTCCTT | YQGLSTATKD |
| | | GATAAAAGGCGCGGGAGAGATCCAGA | TYDALHMQAL |
| | | AATGGGCGGAAAACCACGGCGAAAGA | PPR (SEQ ID |
| | | ATCCGCAGGAAGGGTTATATAACGAA | NO: 119) |
| | | CTTCAAAAGGATAAAATGGCTGAAGC | |
| | | TTATTCCGAAATTGGCATGAAAGGAG | |
| | | AGCGACGTAGGGGCAAAGGGCATGAT | |
| | | GGCCTTTACCAAGGGCTCTCAACCGC | |
| | | TACAAAAGATACTTACGACGCTTTAC | |
| | | ATATGCAAGCACTTCCACCCAGG | |
| | | (SEQ ID NO: 103) | |
| DNA | TTTGCTCAGCGTAACTTCTC | ATGGCACTCCCGGTAACCGCCTTATT | MALPVTALLL |
| Template D | CGGGTTACGTGGAGACCAAA | GCTTCCCCTTGCCCTCTTGCTCCACG | PLALLLHAAR |
| (Construct | AGGCTACGGAGACTCGGGCT | CAGCACGCCCCGATATAGTCTTGACT | PDIVLTQSPP |
| D) | ACGGCCCTGGAGCACCTAGG | CAATCCCCACCCAGTTTGGCAATGTC | SLAMSLGKRA |
| | TGCTCCTAAAGACGTTAGAA | ATTAGGCAAACGAGCAACAATTTCAT | TISCRASESV |
| | GTTGTACAAACTCGCCCAAT | GTAGGGCATCCGAAAGTGTAACGATT | TILGSHLIHW |
| | AGGGCCCCCCAACCAGGGGG | TTGGGGAGTCATTTAATTCATTGGTA | YQQKPGQPPT |
| | GTAGCGGGCAAGCACTTCTG | CCAACAAAAGCCTGGACAACCCCCGA | LLIQLASNVQ |
| | TTTCCCCGGTATGATCTCAT | CGCTCTTGATCCAATTAGCATCTAAC | TGVPARFSGS |
| | AGGCTGTACCCACGGCTGAA | GTCCAAACCGGAGTCCCCGCACGATT | GSRTDETLTI |
| | AGAGAGATTATCGTTACCCG | CTCAGGATCCGGTTCCCGGACTGATT | DPVEEDDVAV |
| | CCTCACTACTTCGAGAAGCC | TTACATTAACTATTGATCCGGTAGAG | YYCLQSRTIP |
| | CAGTAATGGTTCATGAAGTT | GAAGATGACGTCGCTGTCTATTATTG | RTFGGGTKLE |
| | GATCTCGTTGACCCGGTGTT | TCTTCAAAGTAGGACGATTCCACGGA | IKGSTSGSGK |
| | TCCCCCACACCAGAAACCTG | CATTCGGTGGCGGAACTAAATTGGAG | PGSGEGSTKG |
| | TGATGGGGGTGGTCATCCCG | ATTAAAGGTTCCACCTCTGGTAGTGG | QIQLVQSGPE |
| | GTCATGGCGACATGACGGAC | GAAACCCGGGTCCGGTGAAGGGTCCA | LKKPGETVKI |
| | CTCCCCGCGCCGGCACAGGG | CTAAAGGCCAAATTCAACTCGTTCAA | SCKASGYTFT |
| | CCTCTTCGGAGGACGAGTGA | TCCGGACCAGAACTGAAGAAGCCAGG | DYSINWVKRA |
| | CATGGATTCAACCGTGAAGA | AGAAACTGTCAAAATAAGCTGTAAAG | PGKGLKWMGW |
| | GCCTATTGAGCTAGTGTTGA | CTTCCGGTTATACATTTACAGATTAT | INTETREPAY |
| | TTCCTCCGCCCCCGTGAATG | TCCATAAATTGGGTGAAAAGGGCGCC | AYDERGRFAF |
| | CGGCTAATCCCAACTCCGGA | AGGAAAAGGGTTAAAGTGGATGGGTT | SLETSASTAY |
| | GCAGGCGGGCCCAAACCAGG | GGATTAATACAGAGACTCGGGAACCT | LQINNLKYED |
| | GTCTGGCCTGTCGTAACGCG | GCATATGCTTATGATTTTAGGGGAAG | TATYFCALDY |
| | AAAGTCTGGAGCGGAACCGA | GTTTGCCTTTTCTCTGGAGACTTCCG | SYAMDYWGQG |
| | CTACTTTCGGGAAGGCGTGT | CTTCAACTGCTTATCTCCAAATTAAT | TSVTVSSAAA |
| | TTCCTTTTGTTCCTTTTATC | AATCTTAAATATGAGGACACAGCAAC | TTTPAPRPPT |
| | AAGTTTTATGGTGACAACTC | ATACTTCTGTGCTTTGGACTATAGTT | PAPTIASQPL |
| | CTGGTAGACGTTTTATTGCG | ATGCTATGGATTACTGGGGACAAGGA | SLRPEACRPA |
| | TTTATTGAGAGATTTCCAAC | ACCAGTGTCACTGTAAGTTCCGCTGC | AGGAVHTRGL |
| | AATTGAACAGACTAGAACCA | TGCGACGACCACTCCTGCACCGCGAC | DFACDIYIWA |
| | CTTGTTTTATCAAACCCTCA | CACCCACTCCTGCCCCTACTATTGCT | PLAGTCGVLL |
| | CAGAATAAGATAACA (SEQ | AGTCAACCACTTAGCTTGCGACCTGA | LSLVITLYCK |
| | ID NO: 18) | GGCATGTCGGCCCGCGGCAGGTGGCG | RGRKKLLYIF |
| | | CAGTCCACACCAGGGGTTTAGACTTT | KQPFMRPVQT |
| | | GCTTGTGATATTTATATTTGGGCACC | TQEEDGCSCR |
| | | ACTCGCCGGGACTTGCGGTGTTCTTC | FPEEEEGGCE |
| | | TCTTGTCCCTTGTTATAACTCTTTAT | LRVKFSRSAD |
| | | TGTAAGCGCGGAAGGAAGAAATTGTT | APAYQQGQNQ |
| | | ATATATTTTCAAACAACCTTTTATGC | LYNELNLGRR |
| | | GACCCGTACAAACAACTCAGGAAGAG | EEYDVLDKRR |
| | | GACGGGTGTTCTTGTCGGTTTCCAGA | GRDPEMGGKP |
| | | AGAGGAAGAGGGTGGGTGTGAACTCC | RRKNPQEGLY |
| | | GGGTCAAATTTAGTAGGTCAGCAGAT | NELQKDKMAE |
| | | GCGCCGGCGTACCAACAAGGCCAAAA | AYSEIGMKGE |
| | | CCAACTGTATAATGAACTCAATCTCG | RRRGKGHDGL |
| | | GTAGGCGTGAGGAATATGATGTCCTT | YQGLSTATKD |
| | | GATAAAAGGCGCGGGAGAGATCCAGA | TYDALHMQAL |
| | | AATGGGCGGAAAACCACGGCGAAAGA | PPR (SEQ ID |
| | | ATCCGCAGGAAGGGTTATATAACGAA | NO: 119) |
| | | CTTCAAAAGGATAAAATGGCTGAAGC | |
| | | TTATTCCGAAATTGGCATGAAAGGAG | |
| | | AGCGACGTAGGGGCAAAGGGCATGAT | |
| | | GGCCTTTACCAAGGGCTCTCAACCGC | |
| | | TACAAAAGATACTTACGACGCTTTAC | |
| | | ATATGCAAGCACTTCCACCCAGG | |
| | | (SEQ ID NO: 103) | |
| DNA | ATTCTCGGGCTACGGCCCTG | ATGGCACTCCCGGTAACCGCCTTATT | MALPVTALLL |
| Template E | GAGCCACTCCGGCTCCTAAA | GCTTCCCCTTGCCCTCTTGCTCCACG | PLALLLHAAR |
| (Construct | GATTTAGAAGTTTGAGCACA | CAGCACGCCCCGATATAGTCTTGACT | PDIVLTQSPP |
| E) | CCCGCCCACTAGGGCCCCCC | CAATCCCCACCCAGTTTGGCAATGTC | SLAMSLGKRA |
| | ATCCAGGGGGGCAACGGGCA | ATTAGGCAAACGAGCAACAATTTCAT | TISCRASESV |
| | AGCACTTCTGTTTCCCCGGT | GTAGGGCATCCGAAAGTGTAACGATT | TILGSHLIHW |
| | ATGATCTGATAGGCTGTAAC | TTGGGGAGTCATTTAATTCATTGGTA | YQQKPGQPPT |
| | CACGGCTGAAACAGAGATTA | CCAACAAAAGCCTGGACAACCCCCGA | LLIQLASNVQ |
| | TCGTTATCCGCTTCACTACT | CGCTCTTGATCCAATTAGCATCTAAC | TGVPARFSGS |
| | TCGAGAAGCCTAGTAATGAT | GTCCAAACCGGAGTCCCCGCACGATT | GSRTDFTLTI |
| | GGGTGAAATTGAATCCGTTG | CTCAGGATCCGGTTCCCGGACTGATT | DPVEEDDVAV |
| | ATCCGGTGTCTCCCCCACAC | TTACATTAACTATTGATCCGGTAGAG | YYCLQSRTIP |
| | CAGAAACTCATGATGAGGGT | GAAGATGACGTCGCTGTCTATTATTG | RTFGGGTKLE |
| | TGCCATCCCGGCTACGGCGA | TCTTCAAAGTAGGACGATTCCACGGA | IKGSTSGSGK |
| | CGTAGCGGGCATCCCTGCGC | CATTCGGTGGCGGAACTAAATTGGAG | PGSGEGSTKG |
| | TGGCATGAGGCCTCTTAGGA | ATTAAAGGTTCCACCTCTGGTAGTGG | QIQLVQSGPE |
| | GGACGGATGATATGGATCTT | GAAACCCGGGTCCGGTGAAGGGTCCA | LKKPGETVKI |
| | GTCGTGAAGAGCCTATTGAG | CTAAAGGCCAAATTCAACTCGTTCAA | SCKASGYTFT |
| | CTAGTGTCGACTCCTCCGCC | TCCGGACCAGAACTGAAGAAGCCAGG | DYSINWVKRA |
| | CCCGTGAATGCGGCTAATCC | AGAAACTGTCAAAATAAGCTGTAAAG | PGKGLKWMGW |
| | TAACCCCGGAGCAGGTGGGT | CTTCCGGTTATACATTTACAGATTAT | INTETREPAY |
| | CCAATCCAGGGCCTGGCCTG | TCCATAAATTGGGTGAAAAGGGCGCC | AYDERGRFAF |
| | TCGTAATGCGTAAGTCTGGG | AGGAAAAGGGTTAAAGTGGATGGGTT | SLETSASTAY |
| | ACGGAACCGACTACTTTCGG | GGATTAATACAGAGACTCGGGAACCT | LQINNLKYED |
| | GAAGGCGTGTTTCCATTTGT | GCATATGCTTATGATTTTAGGGGAAG | TATYFCALDY |
| | TCATTATTTGTGTGTTTATG | GTTTGCCTTTTCTCTGGAGACTTCCG | SYAMDYWGQG |
| | GTGACAACTCTGGGTAAACG | CTTCAACTGCTTATCTCCAAATTAAT | TSVTVSSAAA |
| | TTCTATTGCGTTTATTGAGA | AATCTTAAATATGAGGACACAGCAAC | TTTPAPRPPT |
| | GATTCCCAACAATTGAACAA | ATACTTCTGTGCTTTGGACTATAGTT | PAPTIASQPL |
| | ACGAGAACTACCTGTTTTAT | ATGCTATGGATTACTGGGGACAAGGA | SLRPEACRPA |
| | TAAATTTACACAGAGAAGAA | ACCAGTGTCACTGTAAGTTCCGCTGC | AGGAVHTRGL |
| | TTACA (SEQ ID NO: | TGCGACGACCACTCCTGCACCGCGAC | DFACDIYIWA |
| | 16) | CACCCACTCCTGCCCCTACTATTGCT | PLAGTCGVLL |
| | | AGTCAACCACTTAGCTTGCGACCTGA | LSLVITLYCK |
| | | GGCATGTCGGCCCGCGGCAGGTGGCG | RGRKKLLYIF |
| | | CAGTCCACACCAGGGGTTTAGACTTT | KQPFMRPVQT |
| | | GCTTGTGATATTTATATTTGGGCACC | TQEEDGCSCR |
| | | ACTCGCCGGGACTTGCGGTGTTCTTC | FPEEEEGGCE |
| | | TCTTGTCCCTTGTTATAACTCTTTAT | LRVKFSRSAD |
| | | TGTAAGCGCGGAAGGAAGAAATTGTT | APAYQQGQNQ |
| | | ATATATTTTCAAACAACCTTTTATGC | LYNELNLGRR |
| | | GACCCGTACAAACAACTCAGGAAGAG | EEYDVLDKRR |
| | | GACGGGTGTTCTTGTCGGTTTCCAGA | GRDPEMGGKP |
| | | AGAGGAAGAGGGTGGGTGTGAACTCC | RRKNPQEGLY |
| | | GGGTCAAATTTAGTAGGTCAGCAGAT | NELQKDKMAE |
| | | GCGCCGGCGTACCAACAAGGCCAAAA | AYSEIGMKGE |
| | | CCAACTGTATAATGAACTCAATCTCG | RRRGKGHDGL |
| | | GTAGGCGTGAGGAATATGATGTCCTT | YQGLSTATKD |
| | | GATAAAAGGCGCGGGAGAGATCCAGA | TYDALHMQAL |
| | | AATGGGCGGAAAACCACGGCGAAAGA | PPR (SEQ ID |
| | | ATCCGCAGGAAGGGTTATATAACGAA | NO: 119) |
| | | CTTCAAAAGGATAAAATGGCTGAAGC | |
| | | TTATTCCGAAATTGGCATGAAAGGAG | |
| | | AGCGACGTAGGGGCAAAGGGCATGAT | |
| | | GGCCTTTACCAAGGGCTCTCAACCGC | |
| | | TACAAAAGATACTTACGACGCTTTAC | |
| | | ATATGCAAGCACTTCCACCCAGG | |
| | | (SEQ ID NO: 103) | |

Expression of circular RNAs encoding anti-BCMA CAR C0-Cultured with MMIS

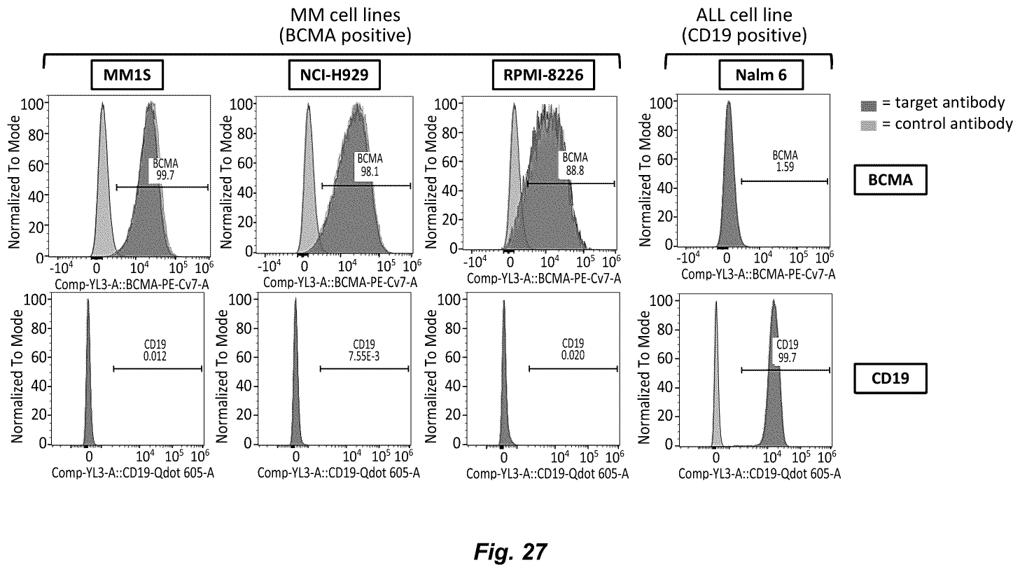

As a preliminary testing of various cell lines used, target protein expression on multiple myeloma positive cells (e.g., MMIS, NCI-H929, and RPMI-8226) and negative target cell line (e.g., Nalm6 target cell line) was analyzed using methods known in the art. Results of the preliminary testing were shown in FIG. 27.

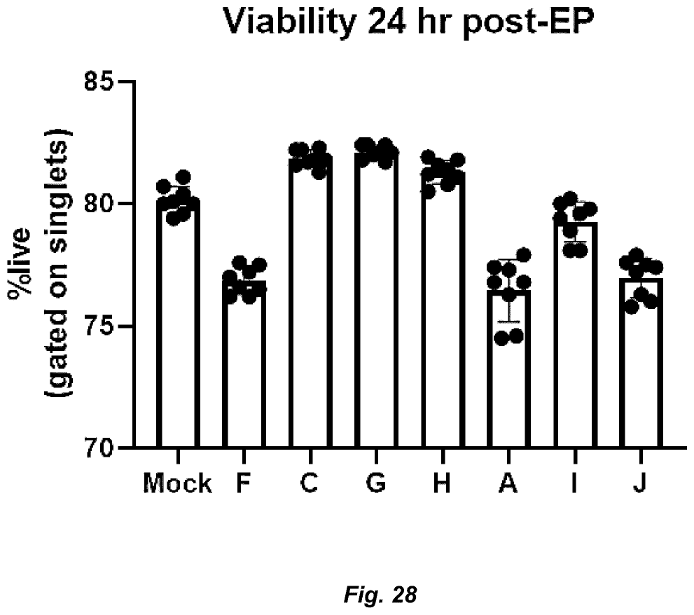

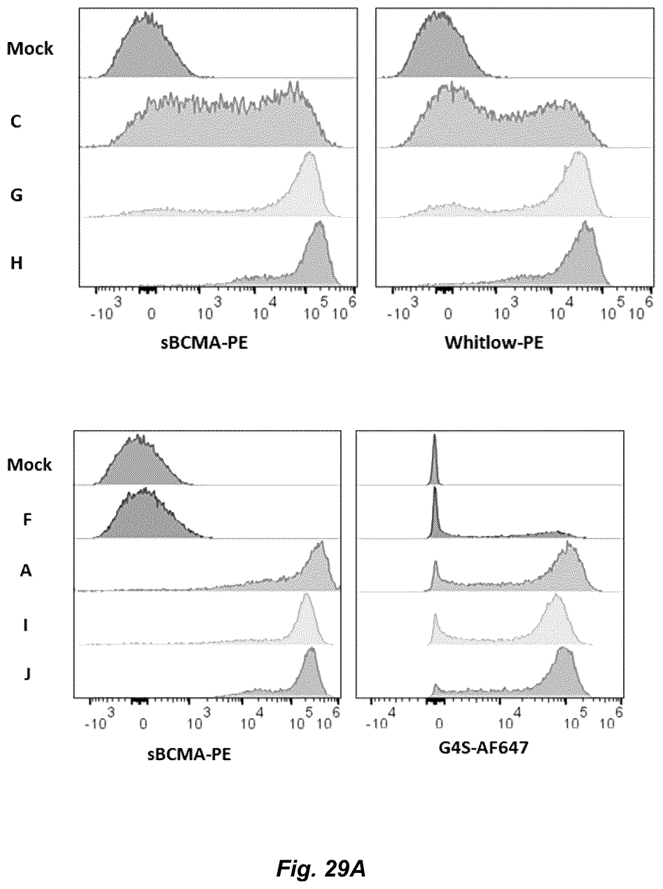

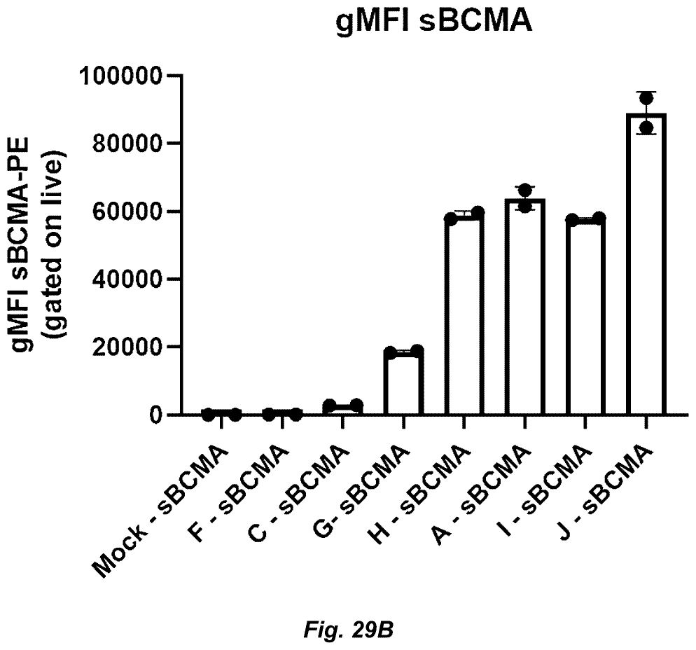

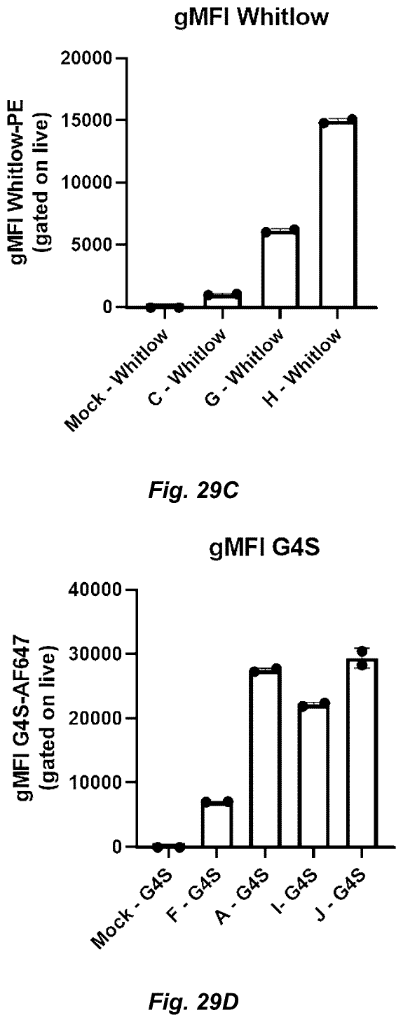

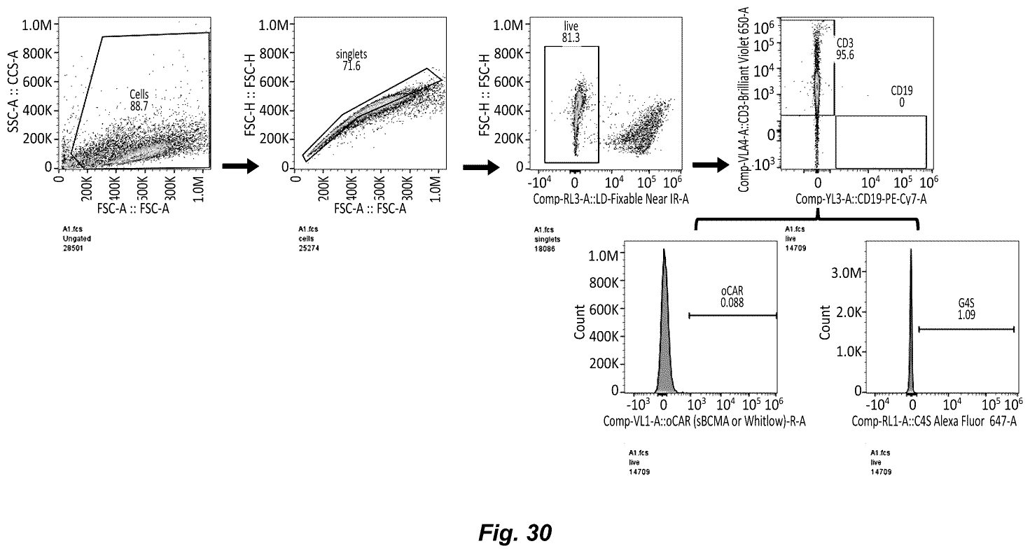

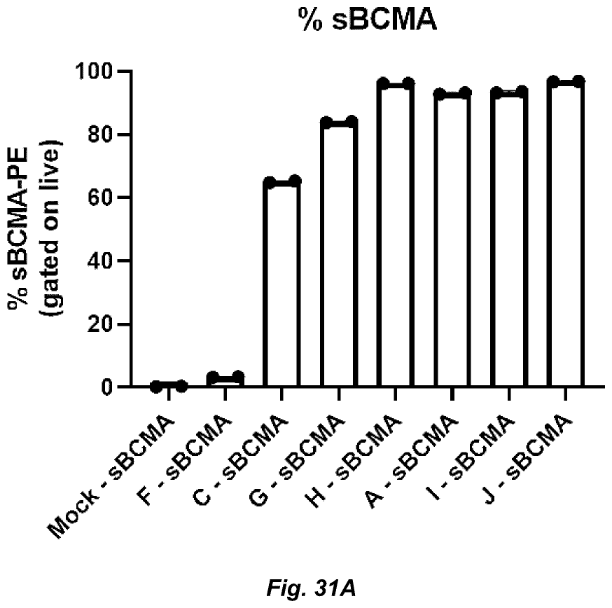

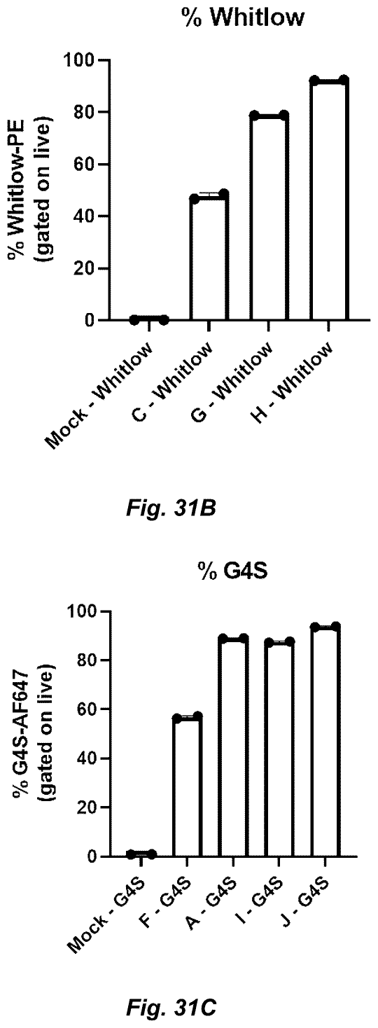

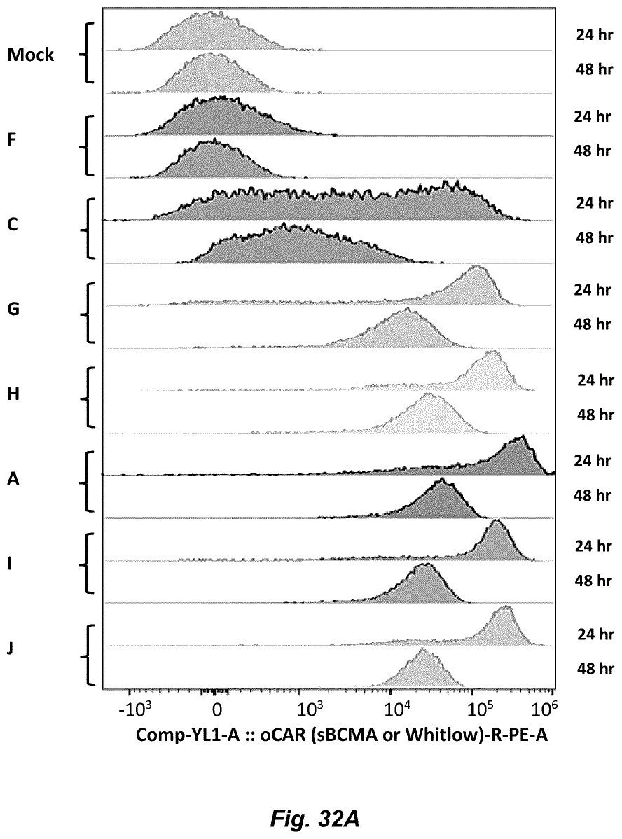

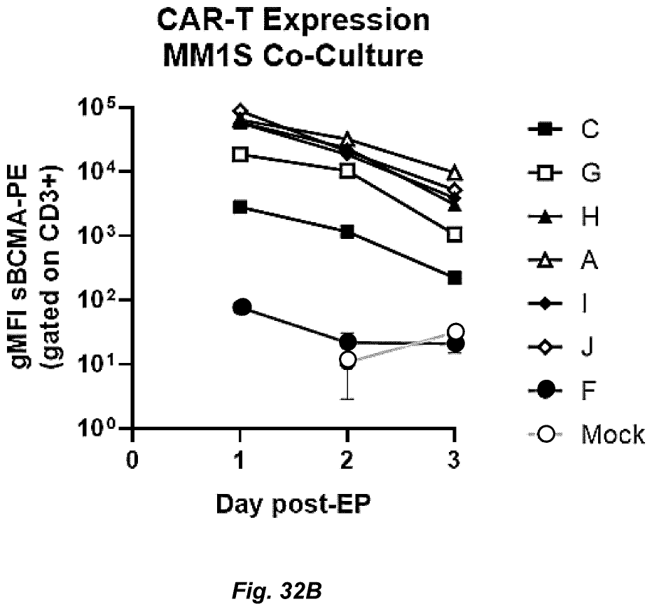

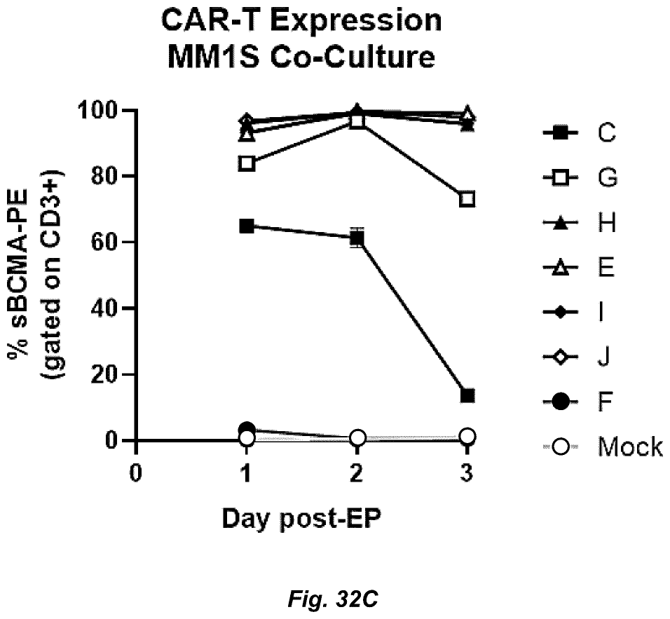

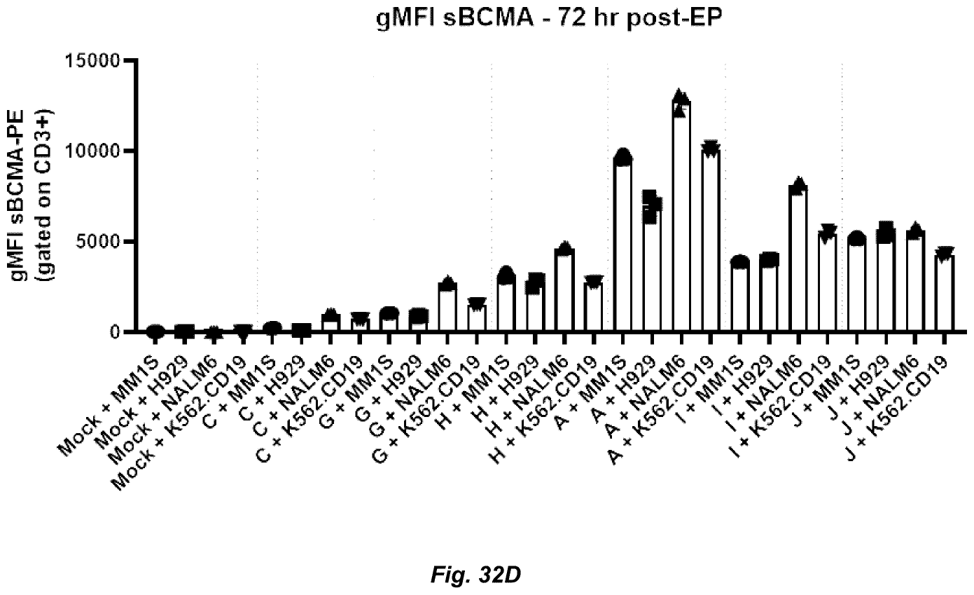

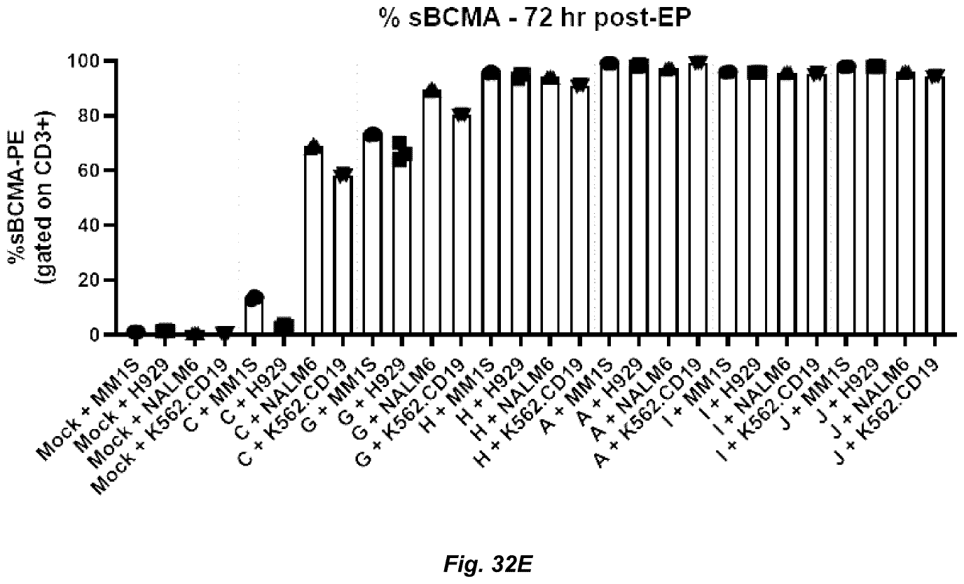

Donor T cells were thawed and activated with anti-CD3/CD28 solutions. Three days after activation, the cells were washed. On the following day, the cells were electroporated with a range of 50 ng dosages of circular RNA encoding a BCMA-41BBζ or BCMA-CD28ζ CAR or CD19-CD28ζ CAR per 0.1×10⁶ T cells to form engineered CAR-T cells (oCAR-T cells). Selected oCAR-T cells were analyzed for percent live cells for 6 circular RNA constructs encoding BCMA CARs and compared to the circular RNA construct comprising a CD19 280 CAR and a control comprising an EP Buffer only (results provided in FIG. 28). Circular RNAs were formed from DNA templates present in Table al and/or B. The cells were given a commercially available soluble BCMA detection reagent containing R-phycoerythrin (PE) fluorophore (e.g., from AcroBiosystems, Delaware), anti-Whitlow PE detection reagent, or anti-G4S linker detection reagent (e.g., G4S-AF647). 24 hours post electroporation of the circular RNA into the T cells, oCAR-T cells were sub-gated on live T cells based on FACS results (e.g., illustrated in FIG. 30) and amount of detectable reagent was collected. Results are provided in FIG. 29A-29D and the table below. From the collected detectable reagent, percent expression was calculated (e.g., percent soluble BCMA-PE, percent anti-Whitlow.PE, and percent G4S-AF647) and provided in FIG. 31A-31C.

The selected oCAR-T cells were co-cultured with targeted multiple myeloma cells (e.g., MMIS), NCI-H929, Nalm6 or K562.CD19 cells at an ET ratio of 1:1 on the day following electroporation of the circular RNAs to the donor T cells. The oCAR-T cells were given soluble BCMA-PE or anti-Whitlow.PE detection reagent. BMCA expression via gMFI was collected (32A-32B and 32D) and percent soluble BCMA-PE was calculated (32C and 32E) at 24-72 hours.

TABLE α2

| ID | Soluble BCMA-PE | | Whitlow-PE | | G4S-AF647 | |
|---|---|---|---|---|---|---|
| Mock | −11.1 | −12.4 | −54.3 | −66.5 | −74.2 | −88.4 |
| DNA Template F | 71.7 | 81.5 | −54.6 | −54.2 | 6973 | 7037 |
| DNA Template G | 18239 | 18887 | 6221 | 6000 | −80.1 | −94.6 |
| DNA Template H | 57811 | 59733 | 14787 | 15078 | −95 | −87.8 |
| DNA Template A | 61524 | 66292 | −73.3 | −85.4 | 27263 | 27709 |
| DNA Template I | 57409 | 58003 | −71.3 | −73.7 | 21881 | 22384 |
| DNA Template J | 84711 | 93417 | −66 | −73.1 | 30438 | 28273 |

TABLE β

| | Internal Ribosome Entry | | |
|---|---|---|---|
| ID | Site (IRES) Sequence | CAR Sequence | CAR Sequence |
| DNA | GTGGCCACGCCCGGGCCA | ATGGCTCTGCCTGTGACAGCTCTGCTGCTGCCTCT | MALPVTALLLP |
| Template F | CCGATACTTCCCTTCACT | GGCTCTGCTTCTGCATGCCGCCAGACCTGACATCC | LALLLHAARPD |
| (Construct | CCTTCGGGACTGTTGGGG | AGATGACCCAGACAACCAGCAGCCTGTCTGCCAGC | IQMTQTTSSLS |
| F) | AGGAACACAACAGGGCTC | CTGGGCGATAGAGTGACCATCAGCTGTAGAGCCAG | ASLGDRVTISC |
| | CCCTGTTTTCCCATTCCT | CCAGGACATCAGCAAGTACCTGAACTGGTATCAGC | RASQDISKYLN |
| | TCCCCCTTTTCCCAACCC | AGAAACCCGACGGCACCGTGAAGCTGCTGATCTAC | WYQQKPDGTVK |
| | CAACCGCCGTATCTGGTG | CACACCAGCAGACTGCACAGCGGCGTGCCAAGCAG | LLIYHTSRLHS |
| | GCGGCAAGACACACGGGT | ATTTTCTGGCAGCGGCTCTGGCACCGACTACAGCC | GVPSRFSGSGS |
| | CTTTCCCTCTAAAGCACA | TGACAATCAGCAACCTGGAACAAGAGGATATCGCT | GTDYSLTISNL |
| | ATTGTGTGTGTGTCCCAG | ACCTACTTCTGCCAGCAAGGCAACACCCTGCCTTA | EQEDIATYFCQ |
| | GTCCTCCTGCGTACGGTG | CACCTTTGGCGGAGGCACCAAGCTGGAAATCACAG | QGNTLPYTFGG |
| | CGGGAGTGCTCCCACCCA | GCGGCGGAGGAAGCGGAGGCGGAGGATCTGGTGGT | GTKLEITGGGG |
| | ACTGTTGTAAGCCTGTCC | GGTGGATCTGAAGTGAAACTGCAAGAGTCTGGCCC | SGGGGSGGGGS |
| | AACGCGTCGTCCTGGCAA | TGGCCTGGTGGCCCCATCTCAATCTCTGAGCGTGA | EVKLQESGPGL |
| | GACTATGACGTCGCATGT | CCTGTACCGTCAGCGGAGTGTCCCTGCCTGATTAT | VAPSQSLSVTC |
| | TCCGCTGCGGATGCCGAC | GGCGTGTCCTGGATCCGGCAGCCTCCTAGAAAAGG | TVSGVSLPDYG |
| | CGGGTAACCGGTTCCCCA | CCTGGAATGGCTGGGCGTGATCTGGGGCAGCGAGA | VSWIRQPPRKG |
| | GTGTGTGTAGTGCGATCT | CAACCTACTACAACAGCGCCCTGAAGTCCCGGCTG | LEWLGVIWGSE |
| | TCCAGGTCCTCCTGGTTG | ACCATCATCAAGGACAACTCCAAGAGCCAGGTGTT | TTYYNSALKSR |
| | GCGTTGTCCAGAAACTGC | CCTGAAGATGAACAGCCTGCAGACCGACGACACCG | LTIIKDNSKSQ |
| | TTCAGGTAAGTGGGGTGT | CCATCTACTATTGCGCCAAGCACTACTACTACGGC | VFLKMNSLQTD |
| | GCCCAATCCCTACAAAGG | GGCAGCTACGCCATGGATTATTGGGGCCAGGGCAC | DTAIYYCAKHY |
| | TTGATTCTTTCACCACCT | CAGCGTGACCGTGTCTAGCATCGAAGTGATGTACC | YYGGSYAMDYW |
| | TAGGAATGCTCCGGAGGT | CTCCACCTTACCTGGACAACGAGAAGTCCAACGGC | GQGTSVTVSSI |
| | ACCCCAGCAACAGCTGGG | ACCATCATCCACGTGAAGGGCAAGCACCTGTGTCC | EVMYPPPYLDN |
| | ATCTGACCGGAGGCTAAT | TTCTCCACTGTTCCCCGGACCTAGCAAGCCTTTCT | EKSNGTIIHVK |
| | TGTCTACGGGTGGTGTTT | GGGTGCTCGTTGTTGTTGGCGGCGTGCTGGCCTGT | GKHLCPSPLFP |
| | CCTTTTTCTTTTCACACA | TACTCTCTGCTGGTTACCGTGGCCTTCATCATCTT | GPSKPFWVLVV |
| | ACTCTACTGCTGACAACT | TTGGGTCCGAAGCAAGCGGAGCCGGCTGCTGCACT | VGGVLACYSLL |
| | CACTGACTATCCACTTGC | CCGACTACATGAACATGACCCCTAGACGGCCCGGA | VTVAFIIFWVR |
| | TCTGTCACG (SEQ ID | CCAACCAGAAAGCACTACCAGCCTTACGCTCCTCC | SKRSRLLHSDY |
| | NO: 17) | TAGAGACTTCGCCGCCTACCGGTCCAGAGTGAAGT | MNMTPRRPGPT |
| | | TCAGCAGATCCGCCGATGCTCCCGCCTATCAGCAG | RKHYQPYAPPR |
| | | GGCCAAAACCAGCTGTACAACGAGCTGAACCTGGG | DFAAYRSRVKF |
| | | GAGAAGAGAAGAGTACGACGTGCTGGACAAGCGGA | SRSADAPAYQQ |
| | | GAGGCAGAGATCCTGAAATGGGCGGCAAGCCCAGA | GQNQLYNELNL |
| | | CGGAAGAATCCTCAAGAGGGCCTGTATAATGAGCT | GRREEYDVLDK |
| | | GCAGAAAGACAAGATGGCCGAGGCCTACAGCGAGA | RRGRDPEMGGK |
| | | TCGGAATGAAGGGCGAGCGCAGAAGAGGCAAGGGA | PRRKNPQEGLY |
| | | CACGATGGACTGTACCAGGGCCTGAGCACCGCCAC | NELQKDKMAEA |
| | | CAAGGATACCTATGATGCCCTGCACATGCAGGCCC | YSEIGMKGERR |
| | | TGCCTCCAAGA (SEQ ID NO: 21) | RGKGHDGLYQG |
| | | | LSTATKDTYDA |
| | | | LHMQALPPR |
| | | | (SEQ ID NO: |
| | | | 29) |
| DNA | CCCCCCTCCCCCCCTTCC | ATGGCCCTCCCCGTCACAGCTCTCCTGCTCCCACT | MALPVTALLLP |
| Template G | CTTCCCTTTGCAACGCAA | GGCCCTTCTTTTGCACGCTGCTCGCCCCGATATCG | LALLLHAARPD |
| (Construct | CAATTGTAAGTGCCCTCA | TGCTCACCCAGTCACCTCCAAGCCTTGCCATGAGC | IVLTQSPPSLA |
| G) | CCTGTCAATTGGGACCAC | CTCGGGAAACGGGCTACCATCTCCTGCCGGGCTTC | MSLGKRATISC |
| | CACTTTCAGTGACCCCAT | AGAGTCCGTCACCATCCTCGGGTCACACCTCATCC | RASESVTILGS |
| | GCGAAGTGCTGAGAGAAA | ACTGGTACCAACAGAAACCAGGGCAGCCTCCTACC | HLIHWYQQKPG |
| | GGAAGCTTTCTTACCCTT | CTCTTGATCCAGTTGGCCTCCAACGTGCAAACTGG | QPPTLLIQLAS |
| | CATTTGTGAACCCACTGG | GGTTCCCGCCAGGTTCAGTGGCTCCGGATCCCGGA | NVQTGVPARFS |
| | TCTAAGCCGCTTGGAATA | CAGATTTCACACTTACCATCGATCCTGTGGAGGAG | GSGSRTDFTLT |
| | CGATGAGTGGAAAAGTTC | GACGATGTGGCCGTCTATTACTGCCTGCAGTCTCG | IDPVEEDDVAV |
| | ATTCTTAATGGAGTGAAA | CACCATCCCTCGGACCTTCGGTGGAGGCACCAAGC | YYCLQSRTIPR |
| | CATGCTTAAATTTCCAGC | TCGAGATCAAGGGTAGCACCTCCGGCTCTGGAAAG | TFGGGTKLEIK |
| | TCGTGCTGGTCTTTCCAG | CCAGGCTCTGGTGAGGGTTCTACCAAGGGCCAAAT | GSTSGSGKPGS |
| | TACGGGGCGGCCCTGTCT | CCAGCTGGTCCAGTCTGGGCCCGAGCTGAAGAAAC | GEGSTKGQIQL |
| | GGCCGTAATTCTTCAGAG | CCGGGGAGACCGTGAAGATCTCCTGCAAGGCCTCC | VQSGPELKKPG |
| | TGTCACGCCACACTTGTG | GGTTATACCTTCACCGACTACTCCATCAACTGGGT | ETVKISCKASG |
| | GATCTCACGTGCCACATG | CAAGCGCGCTCCTGGAAAGGGCCTCAAGTGGATGG | YTFTDYSINWV |
| | ACAGCGCTACAGCTGGAA | GCTGGATCAACACCGAAACCCGCGAGCCTGCCTAT | KRAPGKGLKWM |
| | CTGGGTGCTTGGTGCCCA | GCTTACGACTTCAGGGGCCGGTTCGCTTTCTCACT | GWINTETREPA |
| | TGGAGTAACAGCGAAAAG | GGAGACCTCCGCTTCCACAGCCTACCTCCAGATCA | YAYDFRGRFAF |
| | TGTTAGATCAAGCCTTGC | ACAACCTCAAGTACGAAGACACCGCCACCTATTTC | SLETSASTAYL |
| | TTGGGCTATGAGCCTGCG | TGCGCTCTCGACTATTCCTACGCTATGGACTACTG | QINNLKYEDTA |
| | GAACAACAACTGGTAACA | GGGTCAGGGCACCTCTGTGACCGTCTCTAGCGCAG | TYFCALDYSYA |
| | GTTGCCTCAGGGGCCGAA | CCGCCACCACAACACCAGCCCCACGGCCACCTACT | MDYWGQGTSVT |
| | AGCCACGGTGTTAACAGC | CCCGCACCCACCATCGCATCCCAACCACTCAGTCT | VSSAAATTTPA |
| | ACCCTCATAGTTTGATCC | GAGGCCCGAGGCCTGTAGACCTGCTGCTGGAGGCG | PRPPTPAPTIA |
| | ACCTCAGGGTGGTGATGT | CAGTGCATACCCGCGGTCTCGACTTCGCCTGCGAC | SQPLSLRPEAC |
| | TTAGCAGTTAGTAGTTGC | ATCTATATCTGGGCCCCATTGGCAGGTACCTGTGG | RPAAGGAVHTR |
| | CAATCTGTGTTCACTGAA | CGTGCTGCTGCTGTCACTCGTCATCACCCTGTACT | GLDFACDIYIW |
| | ATCTCGGCATACCGTGTA | GCCGGAGTAAGCGCTCTAGGCTGTTGCACAGCGAC | APLAGTCGVLL |
| | GTGTACAGGGGTGAAGGA | TACATGAACATGACCCCAAGAAGACCAGGGCCTAC | LSLVITLYCRS |
| | TGCCCAGAAGGTACCCGT | CCGGAAGCACTACCAGCCATACGCACCTCCCCGGG | KRSRLLHSDYM |
| | AGGTAACCTTAAGAGACT | ACTTTGCCGCCTATCGGTCTCGGGTGAAGTTCTCA | NMTPRRPGPTR |
| | ATGGATCTGATCTGGGGC | CGCTCCGCTGATGCCCCAGCATACCAGCAGGGGCA | KHYQPYAPPRD |
| | CTTGTCCGGAGTGCTTTA | GAACCAGCTGTACAATGAGCTCAACCTCGGTCGCC | FAAYRSRVKFS |
| | CACACGGCTCAAGGTTAA | GCGAAGAGTACGACGTGCTCGACAAGAGAAGGGGC | RSADAPAYQQG |
| | AAAACGTCTAGCCCCACA | AGGGACCCTGAGATGGGAGGCAAGCCCCGCAGAAA | QNQLYNELNLG |
| | GAGCCCGAGGGATTCGGG | GAATCCCCAGGAAGGTCTGTACAACGAGCTGCAAA | RREEYDVLDKR |
| | TTTTCCCTTTAAAAACCC | AGGATAAGATGGCTGAGGCCTACAGCGAGATCGGC | RGRDPEMGGKP |
| | GACTAGAGCTTATGGTGA | ATGAAGGGCGAAAGGAGACGGGGAAAGGGCCACGA | RRKNPQEGLYN |
| | CAATTATTGCTGTTCAGA | CGGGCTCTACCAGGGACTCTCCACCGCCACCAAGG | ELQKDKMAEAY |
| | CGAACAGTGTAATTGTTG | ACACCTACGACGCCCTCCACATGCAGGCTCTGCCA | SEIGMKGERRR |
| | TCTATTCACAGCAGTTCT | CCCAGG (SEQ ID NO: 104) | GKGHDGLYQGL |
| | ATCAGAGCTTTTCCCACA | | STATKDTYDAL |
| | ACGGATCTTCTTGGCAAG | | HMQALPPR |
| | CAAATACAGCAGGAGTCA | | (SEQ ID NO: |
| | AT (SEQ ID NO: 8) | | 120) |
| DNA | CCCCCCTCCCCCCCTTCC | ATGGCACTTCCCGTCACCGCTCTCCTGCTGCCCCT | MALPVTALLLP |
| Template H | CTTCCCTTTGCAACGCAA | CGCACTGCTGCTCCATGCAGCCCGCCCAGACATCG | LALLLHAARPD |
| (Construct | CAATTGTAAGTGCCCTCA | TCCTGACCCAGTCCCCTCCCTCCCTCGCAATGTCC | IVLTQSPPSLA |
| H) | CCTGTCAATTGGGACCAC | CTCGGGAAACGGGCCACCATCAGCTGCCGGGCCTC | MSLGKRATISC |
| | CACTTTCAGTGACCCCAT | TGAGTCAGTGACAATCCTCGGAAGCCATCTGATCC | RASESVTILGS |
| | GCGAAGTGCTGAGAGAAA | ATTGGTACCAGCAGAAACCCGGTCAGCCTCCAACC | HLIHWYQQKPG |
| | GGAAGCTTTCTTACCCTT | CTCCTCATCCAGCTGGCCTCCAACGTGCAGACAGG | QPPTLLIQLAS |
| | CATTTGTGAACCCACTGG | AGTCCCCGCTCGGTTCTCAGGCAGCGGTTCCAGGA | NVQTGVPARES |
| | TCTAAGCCGCTTGGAATA | CCGACTTCACCCTGACCATCGACCCCGTGGAAGAG | GSGSRTDFTLT |
| | CGATGAGTGGAAAAGTTC | GACGATGTGGCTGTGTACTACTGCCTCCAGTCCCG | IDPVEEDDVAV |
| | ATTCTTAATGGAGTGAAA | GACCATCCCACGGACCTTCGGAGGTGGGACAAAGC | YYCLQSRTIPR |
| | CATGCTTAAATTTCCAGC | TGGAGATCAAAGGCAGCACCAGCGGTTCTGGCAAG | TFGGGTKLEIK |
| | TCGTGCTGGTCTTTCCAG | CCAGGGTCAGGTGAGGGGAGCACAAAGGGTCAGAT | GSTSGSGKPGS |
| | TACGGGGCGGCCCTGTCT | CCAGCTGGTGCAGAGCGGTCCCGAGCTGAAGAAGC | GEGSTKGQIQL |
| | GGCCGTAATTCTTCAGAG | CCGGGGAGACCGTTAAGATCTCCTGCAAGGCTAGC | VQSGPELKKPG |
| | TGTCACGCCACACTTGTG | GGGTACACCTTCACCGACTATAGTATCAACTGGGT | ETVKISCKASG |
| | GATCTCACGTGCCACATG | CAAGCGCGCTCCTGGCAAGGGGCTCAAGTGGATGG | YTFTDYSINWV |
| | ACAGCGCTACAGCTGGAA | GGTGGATCAACACCGAAACCAGGGAGCCCGCATAC | KRAPGKGLKWM |
| | CTGGGTGCTTGGTGCCCA | GCTTATGACTTTCGGGGCCGGTTCGCCTTTTCCCT | GWINTETREPA |
| | TGGAGTAACAGCGAAAAG | GGAGACCAGCGCCTCTACCGCCTACCTCCAGATCA | YAYDERGRFAF |
| | TGTTAGATCAAGCCTTGC | ACAACCTGAAGTACGAGGACACCGCCACCTACTTC | SLETSASTAYL |
| | TTGGGCTATGAGCCTGCG | TGCGCACTCGACTACTCCTACGCTATGGACTACTG | QINNLKYEDTA |
| | GAACAACAACTGGTAACA | GGGTCAGGGTACCTCCGTCACCGTCTCCAGCATCG | TYFCALDYSYA |
| | GTTGCCTCAGGGGCCGAA | AGGTCATGTACCCTCCTCCCTACCTGGACAACGAG | MDYWGQGTSVT |
| | AGCCACGGTGTTAACAGC | AAGTCCAACGGCACCATCATCCATGTGAAGGGCAA | VSSIEVMYPPP |
| | ACCCTCATAGTTTGATCC | GCATCTCTGCCCCAGCCCACTGTTCCCCGGACCCT | YLDNEKSNGTI |
| | ACCTCAGGGTGGTGATGT | CTAAGCCCTTCTGGGTCCTGGTCGTCGTCGGCGGT | IHVKGKHLCPS |
| | TTAGCAGTTAGTAGTTGC | GTTCTGGCTTGCTACAGCTTGCTGGTCACCGTCGC | PLFPGPSKPFW |
| | CAATCTGTGTTCACTGAA | CTTCATCATCTTCTGGGTGCGCTCCAAGAGGAGCC | VLVVVGGVLAC |
| | ATCTCGGCATACCGTGTA | GGCTGCTGCATAGCGACTACATGAACATGACCCCT | YSLLVTVAFII |
| | GTGTACAGGGGTGAAGGA | AGAAGGCCTGGTCCAACCCGCAAGCACTACCAGCC | FWVRSKRSRLL |
| | TGCCCAGAAGGTACCCGT | TTACGCCCCTCCACGGGACTTCGCAGCCTACCGGT | HSDYMNMTPRR |
| | AGGTAACCTTAAGAGACT | CACGGGTGAAGTTCTCTCGGAGCGCAGATGCCCCA | PGPTRKHYQPY |
| | ATGGATCTGATCTGGGGC | GCATACCAGCAGGGCCAGAACCAGCTGTACAACGA | APPRDFAAYRS |
| | CTTGTCCGGAGTGCTTTA | ACTTAACCTTGGTCGGGGGGAGGAATACGATGTGC | RVKFSRSADAP |
| | CACACGGCTCAAGGTTAA | TGGACAAGCGCAGGGGTCGGGATCCTGAAATGGGC | AYQQGQNQLYN |
| | AAAACGTCTAGCCCCACA | GGGAAACCACGCCGGAAGAACCCACAGGAGGGGCT | ELNLGRREEYD |
| | GAGCCCGAGGGATTCGGG | CTATAACGAGCTCCAAAAGGATAAGATGGCTGAGG | VLDKRRGRDPE |
| | TTTTCCCTTTAAAAACCC | CTTACAGCGAGATTGGAATGAAGGGAGAAAGAAGA | MGGKPRRKNPQ |
| | GACTAGAGCTTATGGTGA | CGGGGCAAGGGTCACGACGGGTTGTACCAGGGTCT | EGLYNELQKDK |
| | CAATTATTGCTGTTCAGA | GAGCACCGCCACCAAGGACACCTACGACGCCCTCC | MAEAYSEIGMK |
| | CGAACAGTGTAATTGTTG | ACATGCAAGCCCTTCCACCCCGC (SEQ ID NO: | GERRRGKGHDG |
| | TCTATTCACAGCAGTTCT | 105) | LYQGLSTATKD |
| | ATCAGAGCTTTTCCCACA | | TYDALHMQALP |
| | ACGGATCTTCTTGGCAAG | | PR (SEQ ID |
| | CAAATACAGCAGGAGTCA | | NO: 121) |
| | AT (SEQ ID NO: 8) | | |
| DNA | CCCCCCTCCCCCCCTTCC | ATGGCACTCCCCGTTACCGCCCTTCTGCTGCCTCT | MALPVTALLLP |
| Template I | CTTCCCTTTGCAACGCAA | CGCCTTGCTGCTGCACGCAGCCAGACCACAGGTCA | LALLLHAARPQ |
| (Construct | CAATTGTAAGTGCCCTCA | AGCTGGAGGAGTCTGGTGGCGGGCTCGTTCAAGCA | VKLEESGGGLV |
| | CCTGTCAATTGGGACCAC | GGTCGGAGTCTCCGCCTGTCTTGCGCAGCATCAGA | QAGRSLRLSCA |
| | CACTTTCAGTGACCCCAT | GCATACCTTCTCCTCACACGTGATGGGGTGGTTCA | ASEHTFSSHVM |
| | GCGAAGTGCTGAGAGAAA | GGCAAGCTCCCGGTAAGGAGAGGGAGTCCGTGGCC | GWFRQAPGKER |
| | GGAAGCTTTCTTACCCTT | GTTATCGGCTGGCGCGATATCAGCACCTCCTACGC | ESVAVIGWRDI |
| | CATTTGTGAACCCACTGG | AGACAGCGTTAAGGGCCGGTTCACTATCTCCAGGG | STSYADSVKGR |
| | TCTAAGCCGCTTGGAATA | ACAACGCTAAGAAGACACTCTACCTCCAGATGAAC | FTISRDNAKKT |
| | CGATGAGTGGAAAAGTTC | AGTCTGAAGCCCGAGGACACCGCAGTGTACTATTG | LYLQMNSLKPE |
| | ATTCTTAATGGAGTGAAA | CGCTGCTCGGCGGATCGATGCTGCCGACTTCGACA | DTAVYYCAARR |
| | CATGCTTAAATTTCCAGC | GCTGGGGTCAAGGGACCCAGGTCACCGTTTCCAGC | IDAADFDSWGQ |
| | TCGTGCTGGTCTTTCCAG | GGAGGTGGCGGAAGTGGTGGCGGAGGATCAGGTGG | GTQVTVSSGGG |
| | TACGGGGCGGCCCTGTCT | TGGAGGCTCCGAGGTCCAGCTGGTGGAATCAGGAG | GSGGGGSGGGG |
| | GGCCGTAATTCTTCAGAG | GCGGCTTGGTGCAGGCTGGTGGGTCTTTGCGGTTG | SEVQLVESGGG |
| | TGTCACGCCACACTTGTG | TCCTGCGCAGCTTCCGGCAGGACCTTCACCATGGG | LVQAGGSLRLS |
| | GATCTCACGTGCCACATG | ATGGTTCAGACAAGCCCCAGGTAAGGAGCGGGAGT | CAASGRTFTMG |
| | ACAGCGCTACAGCTGGAA | TTGTGGCCGCAATCTCACTGTCTCCCACCCTCGCT | WFRQAPGKERE |
| | CTGGGTGCTTGGTGCCCA | TACTACGCCGAGAGTGTGAAGGGGCGCTTCACAAT | FVAAISLSPTL |
| | TGGAGTAACAGCGAAAAG | CAGTCGCGACAACGCAAAGAACACCGTCGTCCTGC | AYYAESVKGRE |
| | TGTTAGATCAAGCCTTGC | AAATGAACTCCCTGAAGCCTGAGGATACCGCACTC | TISRDNAKNTV |
| | TTGGGCTATGAGCCTGCG | TATTACTGCGCCGCCGATCGGAAGAGCGTCATGTC | VLQMNSLKPED |
| | GAACAACAACTGGTAACA | CATCCGGCCCGACTATTGGGGCCAAGGCACCCAAG | TALYYCAADRK |
| | GTTGCCTCAGGGGCCGAA | TGACCGTCAGCTCCACCTCCACAACCACTCCCGCC | SVMSIRPDYWG |
| | AGCCACGGTGTTAACAGC | CCAAGACCACCTACCCCAGCCCCAACAATCGCATC | QGTQVTVSSTS |
| | ACCCTCATAGTTTGATCC | CCAGCCTCTGTCCCTTCGGCCCGAAGCTTGTCGCC | TTTPAPRPPTP |
| | ACCTCAGGGTGGTGATGT | CTGCAGCAGGTGGAGCAGTGCACACCCGGGGACTG | APTIASQPLSL |
| | TTAGCAGTTAGTAGTTGC | GACTTCGCCTGCGACATCTACATCTGGGCACCCCT | RPEACRPAAGG |
| | CAATCTGTGTTCACTGAA | GGCTGGAACCTGCGGCGTGTTGCTGCTGAGCCTGG | AVHTRGLDFAC |
| | ATCTCGGCATACCGTGTA | TGATCACCCTCTACTGCCGCTCTAAGAGAAGCCGG | DIYIWAPLAGT |
| | GTGTACAGGGGTGAAGGA | CTGCTGCATAGCGACTACATGAACATGACCCCTAG | CGVLLLSLVIT |
| | TGCCCAGAAGGTACCCGT | GAGACCAGGACCCACCCGGAAGCACTACCAGCCTT | LYCRSKRSRLL |
| | AGGTAACCTTAAGAGACT | ACGCTCCTCCACGGGATTTCGCTGCTTACCGCAGC | HSDYMNMTPRR |
| | ATGGATCTGATCTGGGGC | CGGGTGAAGTTTTCCAGGTCAGCTGACGCCCCTGC | PGPTRKHYQPY |
| | CTTGTCCGGAGTGCTTTA | CTACCAGCAGGGCCAGAACCAATTGTACAACGAAC | APPRDFAAYRS |
| | CACACGGCTCAAGGTTAA | TGAATCTGGGACGGCGCGAGGAATACGACGTCCTG | RVKFSRSADAP |
| | AAAACGTCTAGCCCCACA | GACAAGAGGCGGGGTAGAGATCCCGAGATGGGCGG | AYQQGQNQLYN |
| | GAGCCCGAGGGATTCGGG | GAAACCTCGGCGGAAGAACCCTCAGGAGGGGCTCT | ELNLGRREEYD |
| | TTTTCCCTTTAAAAACCC | ACAACGAGCTGCAGAAGGATAAGATGGCCGAAGCC | VLDKRRGRDPE |
| | GACTAGAGCTTATGGTGA | TACTCCGAGATCGGGATGAAGGGTGAACGGAGGAG | MGGKPRRKNPQ |
| | CAATTATTGCTGTTCAGA | GGGCAAGGGACACGACGGCCTGTATCAGGGCCTCA | EGLYNELQKDK |
| | CGAACAGTGTAATTGTTG | GCACCGCTACCAAGGACACCTACGACGCCCTGCAC | MAEAYSEIGMK |
| | TCTATTCACAGCAGTTCT | ATGCAGGCTCTCCCACCACGG (SEQ ID NO: | GERRRGKGHDG |
| | ATCAGAGCTTTTCCCACA | 107) | LYQGLSTATKD |
| | ACGGATCTTCTTGGCAAG | | TYDALHMQALP |
| | CAAATACAGCAGGAGTCA | | PR (SEQ ID |
| | AT (SEQ ID NO: 8) | | NO: 123) |
| DNA | CCCCCCTCCCCCCCTTCC | ATGGCTCTTCCCGTCACCGCTTTGCTGCTGCCCCT | MALPVTALLLP |
| Template J | CTTCCCTTTGCAACGCAA | GGCACTCCTCCTCCATGCTGCTCGGCCTCAGGTGA | LALLLHAARPQ |
| (Construct | CAATTGTAAGTGCCCTCA | AGCTGGAGGAGAGTGGTGGCGGTCTGGTGCAAGCT | VKLEESGGGLV |
| J | CCTGTCAATTGGGACCAC | GGCAGATCTCTGCGCCTGTCTTGCGCAGCCAGCGA | QAGRSLRISCA |
| | CACTTTCAGTGACCCCAT | ACACACCTTCTCCTCCCACGTGATGGGGTGGTTTC | ASEHTESSHVM |
| | GCGAAGTGCTGAGAGAAA | GGCAGGCACCCGGGAAAGAGCGCGAGTCCGTCGCA | GWFRQAPGKER |
| | GGAAGCTTTCTTACCCTT | GTCATCGGGTGGCGGGACATCTCTACCAGCTACGC | ESVAVIGWRDI |
| | CATTTGTGAACCCACTGG | AGATTCCGTCAAGGGCCGGTTCACCATTTCCCGGG | STSYADSVKGR |
| | TCTAAGCCGCTTGGAATA | ATAACGCTAAGAAGACCCTCTACCTGCAAATGAAC | FTISRDNAKKT |
| | CGATGAGTGGAAAAGTTC | TCTCTGAAGCCCGAAGACACCGCCGTCTACTATTG | LYLQMNSLKPE |
| | ATTCTTAATGGAGTGAAA | CGCAGCAAGGCGCATCGACGCTGCCGACTTCGACT | DTAVYYCAARR |
| | CATGCTTAAATTTCCAGC | CTTGGGGCCAAGGAACCCAGGTCACCGTGTCTTCC | IDAADFDSWGQ |
| | TCGTGCTGGTCTTTCCAG | GGAGGAGGAGGCTCCGGTGGTGGAGGTTCTGGAGG | GTQVTVSSGGG |
| | TACGGGGCGGCCCTGTCT | TGGCGGCTCAGAGGTGCAGCTCGTGGAGAGCGGTG | GSGGGGSGGGG |
| | GGCCGTAATTCTTCAGAG | GTGGACTCGTTCAGGCAGGCGGCAGTTTGCGGCTG | SEVQLVESGGG |
| | TGTCACGCCACACTTGTG | TCCTGTGCAGCCTCCGGTCGCACTTTCACTATGGG | LVQAGGSLRLS |
| | GATCTCACGTGCCACATG | ATGGTTCCGCCAGGCTCCTGGTAAAGAAAGGGAGT | CAASGRTFTMG |
| | ACAGCGCTACAGCTGGAA | TCGTGGCCGCCATCAGTCTGAGCCCCACCCTCGCA | WFRQAPGKERE |
| | CTGGGTGCTTGGTGCCCA | TACTACGCCGAGAGCGTGAAGGGTAGGTTCACTAT | FVAAISLSPTL |
| | TGGAGTAACAGCGAAAAG | CAGCCGGGACAACGCCAAGAACACCGTGGTGCTCC | AYYAESVKGRE |
| | TGTTAGATCAAGCCTTGC | AGATGAATTCCCTGAAGCCTGAGGATACCGCCCTC | TISRDNAKNTV |
| | TTGGGCTATGAGCCTGCG | TACTACTGCGCTGCCGACCGCAAGAGCGTGATGAG | VLQMNSLKPED |
| | GAACAACAACTGGTAACA | CATCCGGCCTGACTATTGGGGTCAGGGGACACAGG | TALYYCAADRK |
| | GTTGCCTCAGGGGCCGAA | TGACCGTCAGCAGCATCGAGGTGATGTATCCACCA | SVMSIRPDYWG |
| | AGCCACGGTGTTAACAGC | CCCTACCTCGACAACGAGAAGTCCAACGGCACCAT | QGTQVTVSSIE |
| | ACCCTCATAGTTTGATCC | CATCCACGTCAAGGGGAAGCACCTCTGCCCTTCCC | VMYPPPYLDNE |
| | ACCTCAGGGTGGTGATGT | CTCTGTTCCCTGGCCCCTCAAAGCCCTTCTGGGTC | KSNGTIIHVKG |
| | TTAGCAGTTAGTAGTTGC | CTGGTGGTGGTTGGTGGGGTGCTGGCTTGCTACTC | KHLCPSPLFPG |
| | CAATCTGTGTTCACTGAA | CCTGCTCGTGACCGTGGCTTTCATCATCTTCTGGG | PSKPFWVLVVV |
| | ATCTCGGCATACCGTGTA | TTCGGAGCAAACGGTCCAGACTGCTGCACTCCGAC | GGVLACYSLLV |
| | GTGTACAGGGGTGAAGGA | TACATGAACATGACCCCAAGAAGACCTGGGCCCAC | TVAFIIFWVRS |
| | TGCCCAGAAGGTACCCGT | ACGGAAGCATTACCAACCCTATGCACCACCTCGGG | KRSRLLHSDYM |
| | AGGTAACCTTAAGAGACT | ATTTCGCCGCCTACAGATCCCGGGTCAAGTTCTCC | NMTPRRPGPTR |
| | ATGGATCTGATCTGGGGC | AGGTCCGCCGATGCACCAGCCTATCAGCAGGGGCA | KHYQPYAPPRD |
| | CTTGTCCGGAGTGCTTTA | AAACCAGCTGTATAATGAGCTGAACCTTGGACGGC | FAAYRSRVKES |
| | CACACGGCTCAAGGTTAA | GCGAGGAGTACGACGTGCTCGACAAAAGACGCGGT | RSADAPAYQQG |
| | AAAACGTCTAGCCCCACA | CGCGACCCAGAGATGGGCGGCAAGCCTAGACGCAA | QNQLYNELNLG |
| | GAGCCCGAGGGATTCGGG | GAATCCCCAGGAGGGGCTCTATAACGAGTTGCAGA | RREEYDVLDKR |
| | TTTTCCCTTTAAAAACCC | AGGATAAGATGGCCGAGGCCTACAGCGAGATCGGG | RGRDPEMGGKP |
| | GACTAGAGCTTATGGTGA | ATGAAAGGCGAAAGACGGCGCGGAAAGGGTCACGA | RRKNPQEGLYN |
| | CAATTATTGCTGTTCAGA | CGGACTCTACCAGGGCCTGAGCACAGCCACCAAAG | ELQKDKMAEAY |
| | CGAACAGTGTAATTGTTG | ACACCTACGACGCTCTGCATATGCAAGCACTGCCT | SEIGMKGERRR |
| | TCTATTCACAGCAGTTCT | CCCCGG (SEQ ID NO: 108) | GKGHDGLYQGL |
| | ATCAGAGCTTTTCCCACA | | STATKDTYDAL |
| | ACGGATCTTCTTGGCAAG | | HMQALPPR |
| | CAAATACAGCAGGAGTCA | | (SEQ ID NO: |
| | AT (SEQ ID NO: 8) | | 124) |

### Example 15: Cytotoxicity Analysis of CAR Expression

BCMA CAR Expression and CD19 CAR Expression on Multiple Myeloma Positive and Negative Target Cell Lines

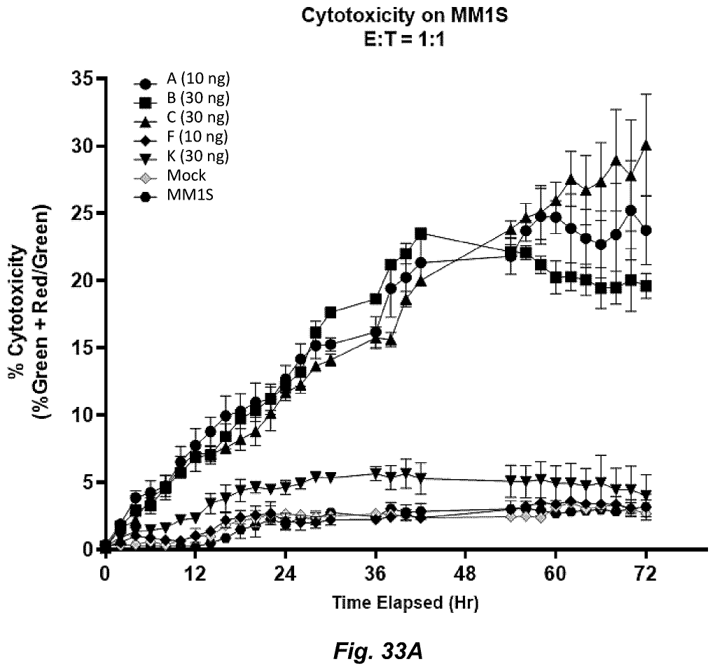

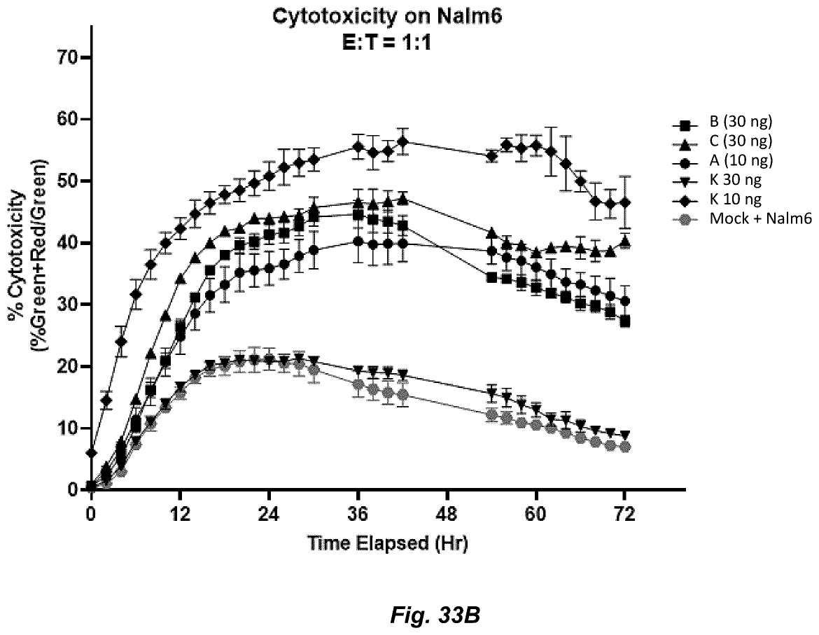

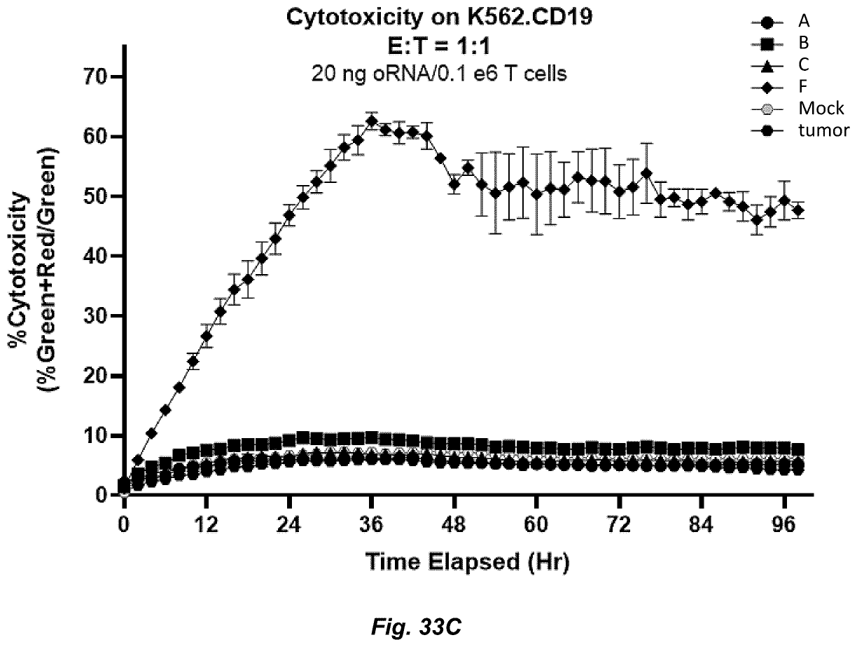

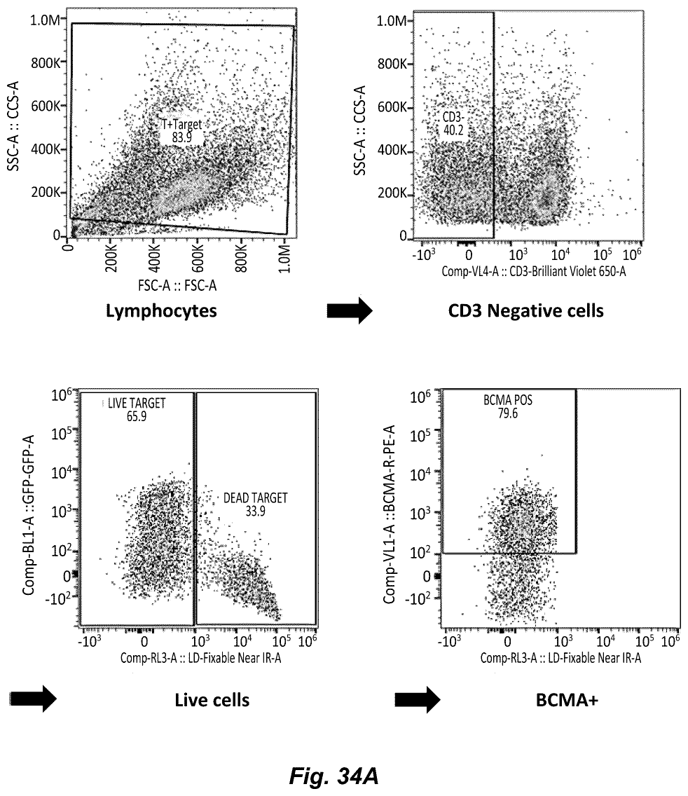

Donor T cells were thawed and activated with anti-CD3/CD28 solutions. Three days after activation, the cells were washed. On the following day, the cells were electroporated with a range of 10-30 ng dosages of circular RNA encoding a BCMA-41BBζ CAR or CD19CD28ζ CAR per 0.1×10⁶ T cells to form engineered CAR-T cells (oCAR-T cells). Circular RNAs comprised in the following order: a ‘3 Anabaena exon, an internal ribosome entry site (IRES), a coding region encoding a BCMA CAR or CD19 CAR, and a 5’ Anabaena exon. Circular RNAs were formed from an IVT reaction of DNA templates from Table al, Table B and Table Y1. As a control, Mock T cells were used, wherein the Mock T cells were not electroporated with any circular RNAs. A day following electroporation of the circular RNAs to the T cells, the oCAR-T cells were co-cultured with target multiple myeloma (MM) positive or negative T cells at a 1:1 E:T ratio. MM positive T cells comprised of MMIS, NCI-H929 or RPM1-8226. MM negative T cells comprised of a CD19 positive T cell line (i.e., Nalm6 cell line). As a control, the target tumor cell without co-culturing with the T cells was analyzed for comparison purposes. The circular RNAs were given a commercially available soluble BCMA detection reagent containing R-phycoerythrin (PE) fluorophore or soluble CD19 detection reagent containing quantum dot (qdot) fluorophore (e.g., from AcroBiosystems, Delaware). The oCAR-T cells were also placed into a commercially available live-cell analysis portfolio system (e.g., an IncuCyte) and analyzed 0-72 or 0-96 hours post co-culture for cytotoxicity. The cytotoxicity results can be seen in FIGS. 33A-33C for each of the circular RNAs encoding BCMA, CD19, or HER2 CARs, respectively. Cross-reactivity for the various circular RNAs encoding the various CARs was observed on MMIS, Nalm6 and CD19 positive T cell line in FIGS. 33A, 33B, and 33C respectively.

### Cytotoxicity of BCMA Targeted Killing on MMIS Via FACS

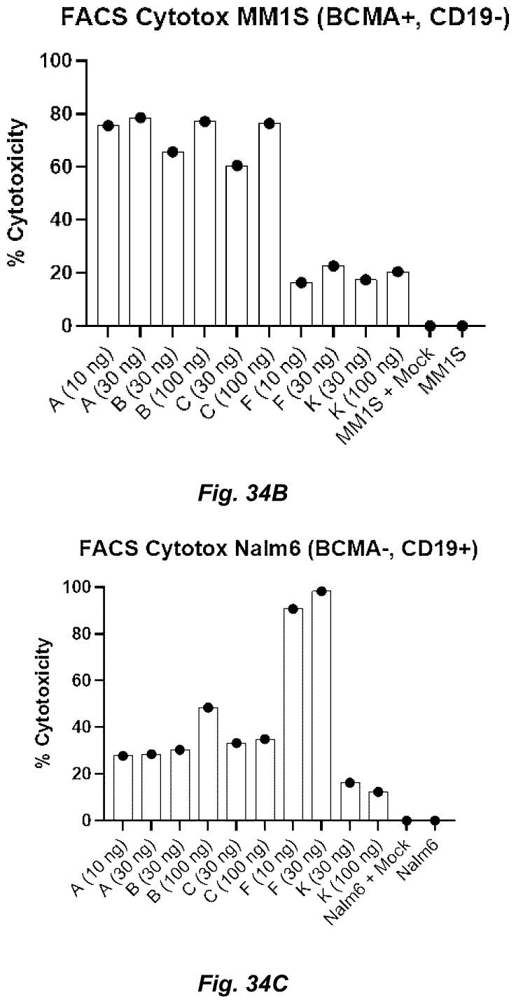

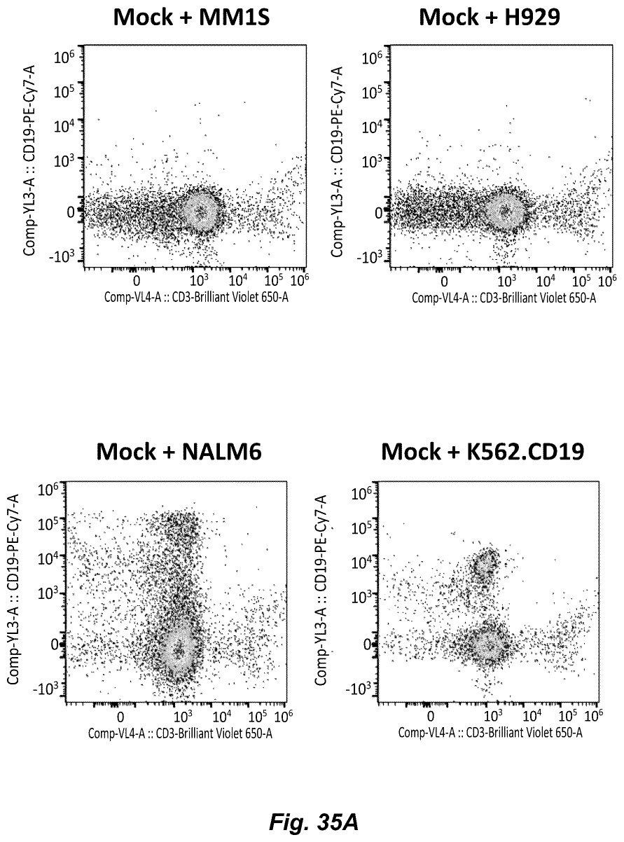

BMCA positive cells were acquired and prepped. The cells were electroporated with 30 ng per 0.1×10⁶ cells of circular RNA comprising a 3′ Anabaena exon, an internal ribosome entry site (IRES), a BCMA-41BB (CAR, and a 5′ Anabaena exon. Circular RNAs were formed from an IVT reaction of DNA templates from Table α1, Table β and Table γ1. On the following day, the cells were then co-cultured with MMIS cells and dyed with FSC-A, CD3-Brilliant Violet 650-A, LD-fixable Near IR-A for fluorescence activated cell sorting analysis (FACS). Resulting FACS imaging for cell post 24 hours after co-culturing with MMIS can be seen in FIG. 34A. From the FACS imaging, percent cell lysis was calculated (e.g., % cell lysis=(1−((% of live Target Cells in Test Sample)/(% of live Target Cells in Control Sample)))×100).

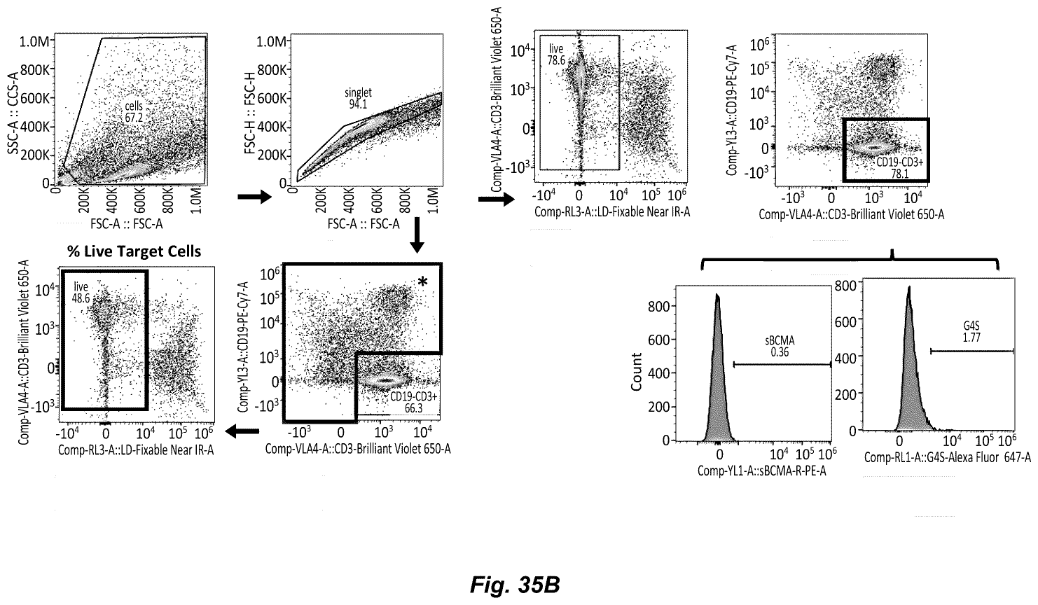

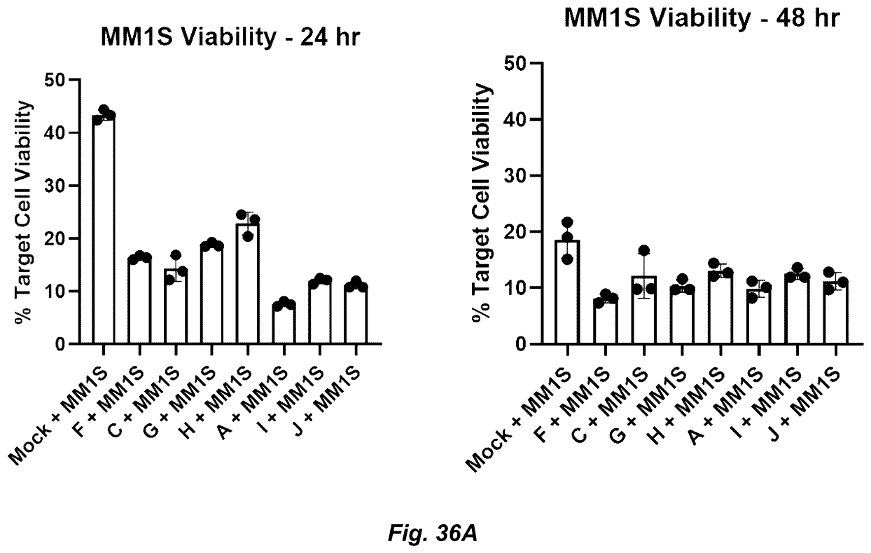

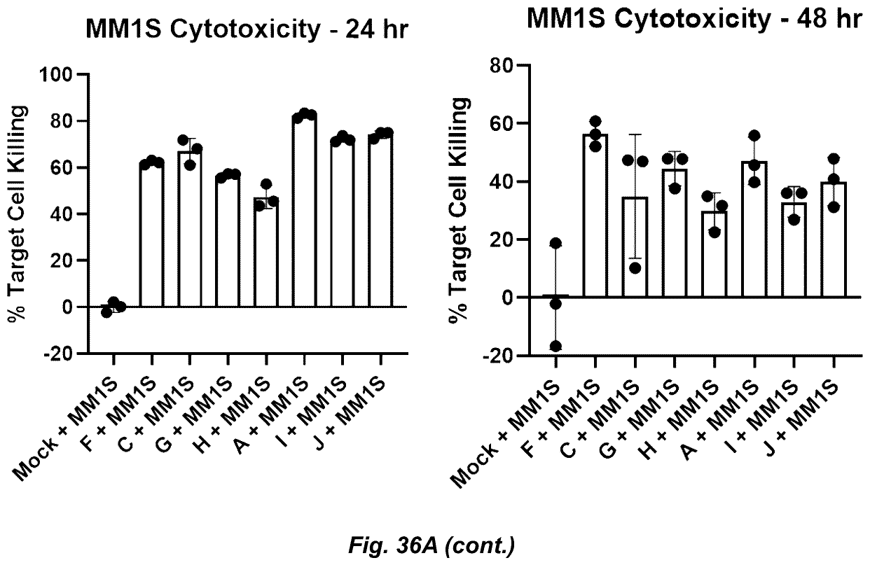

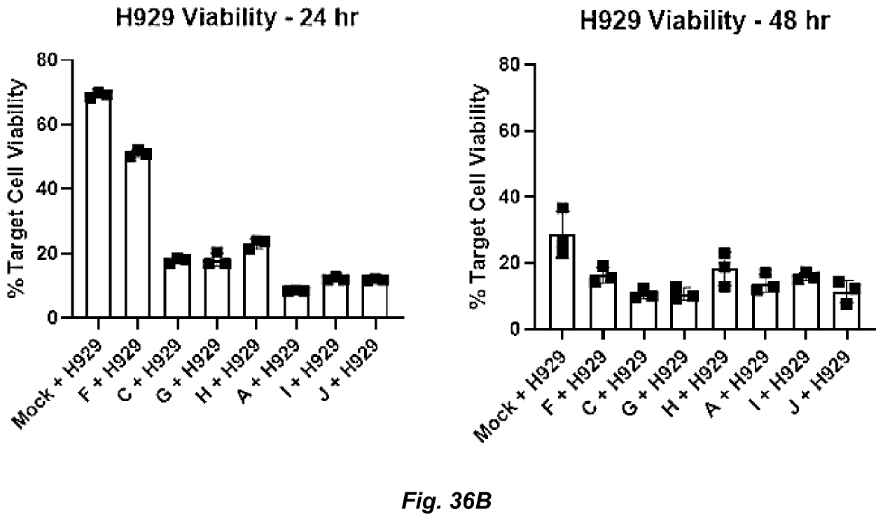

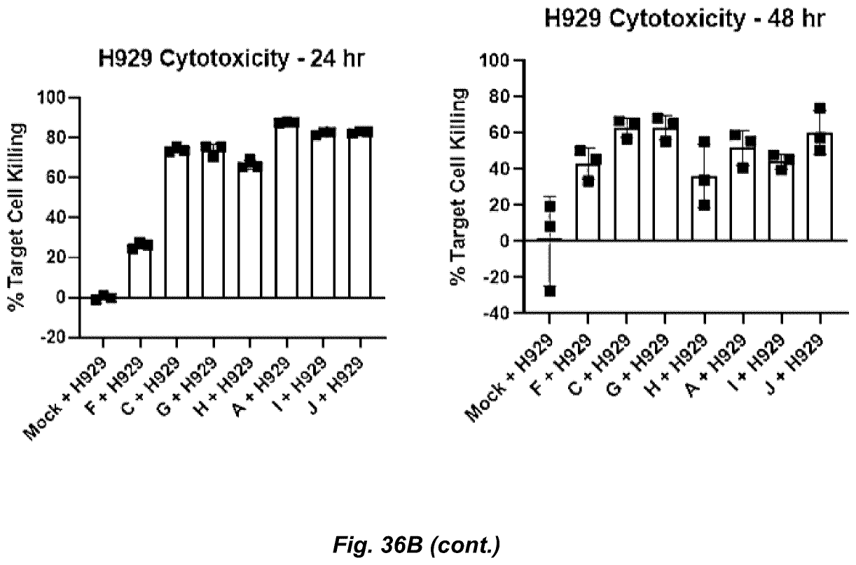

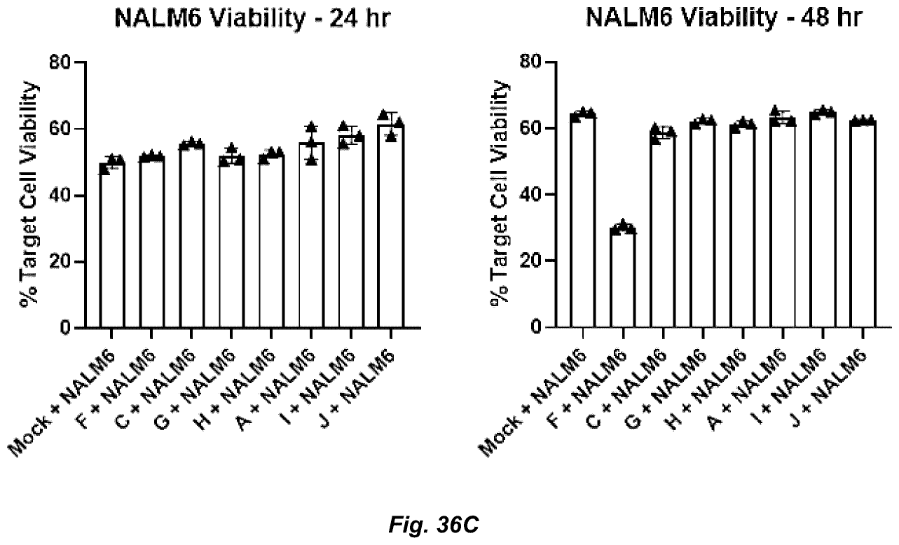

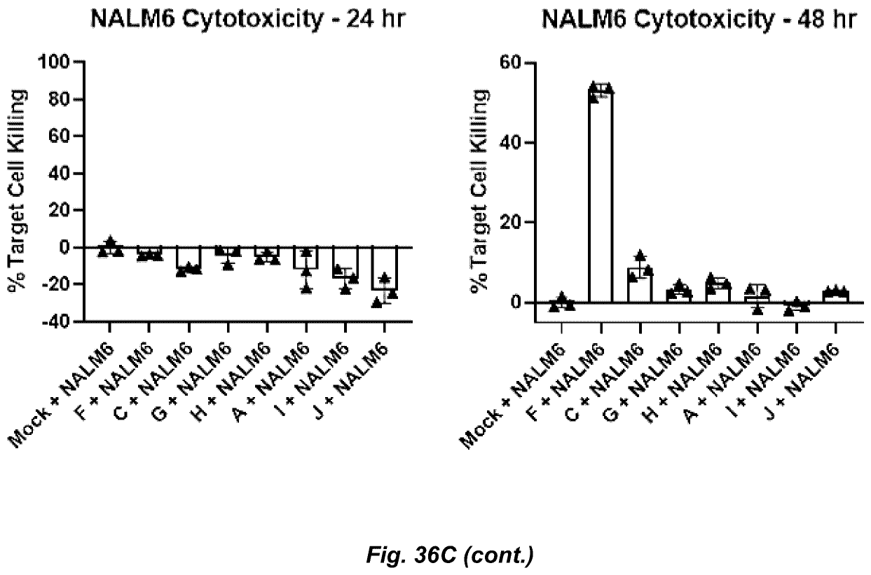

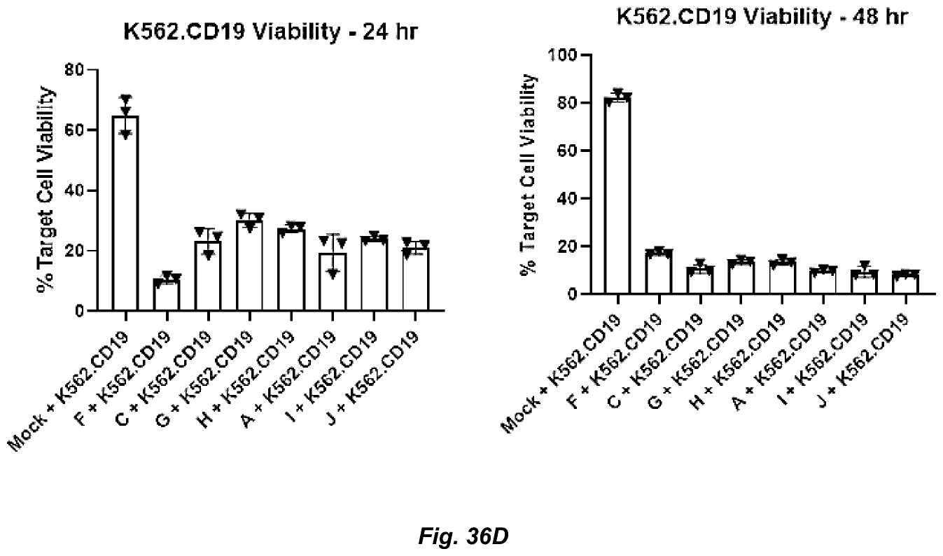

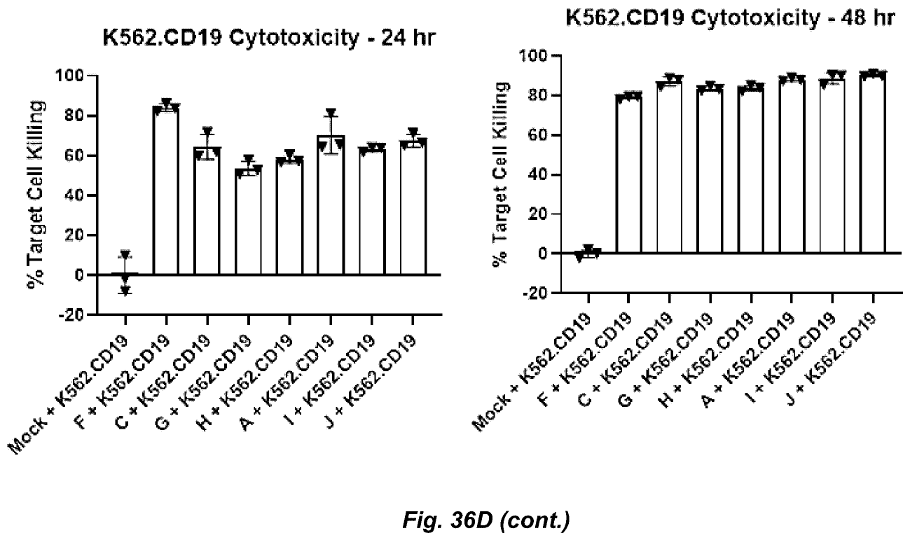

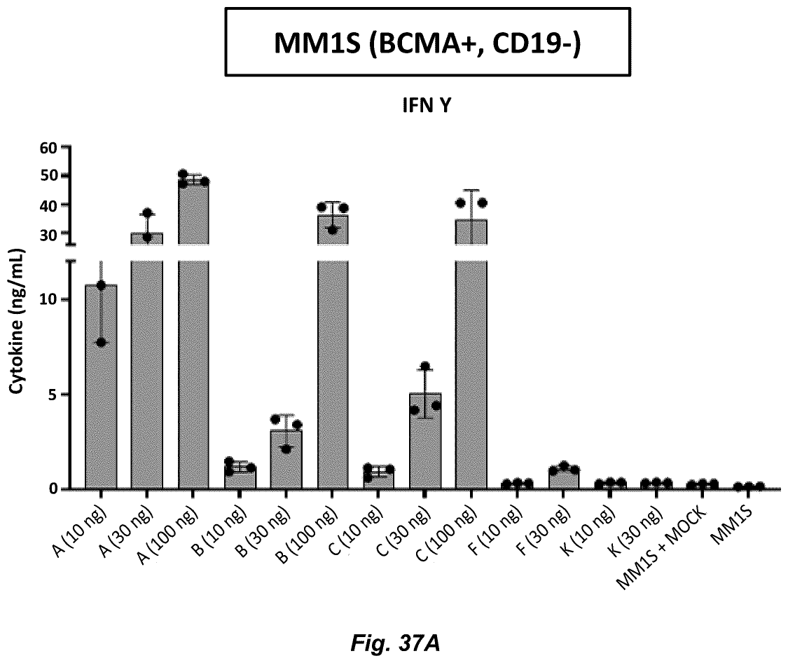

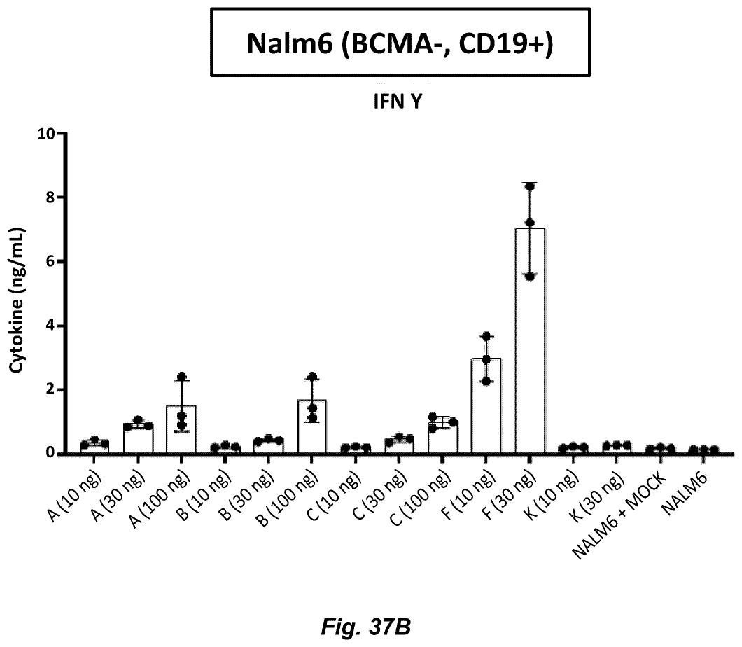

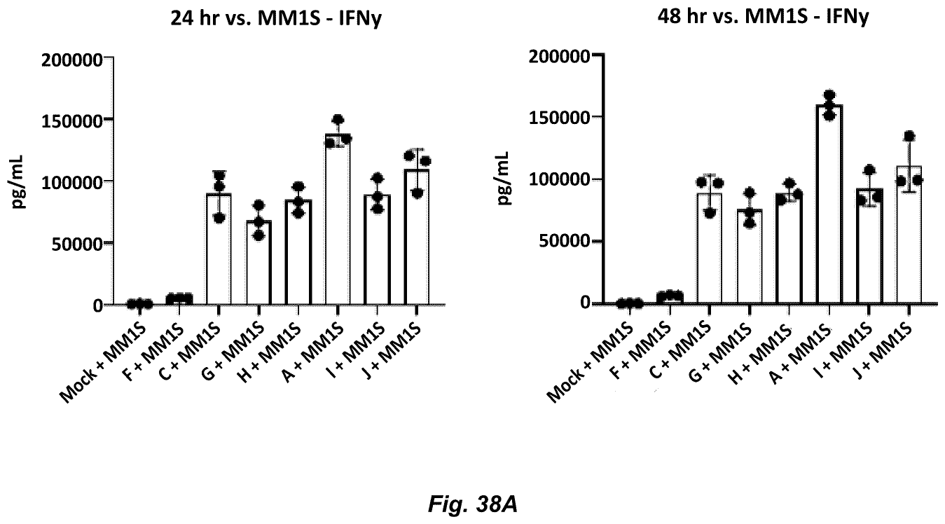

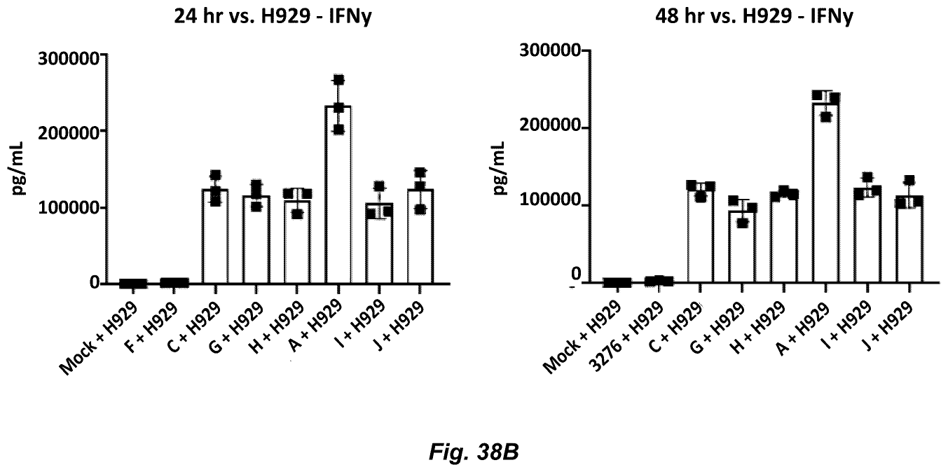

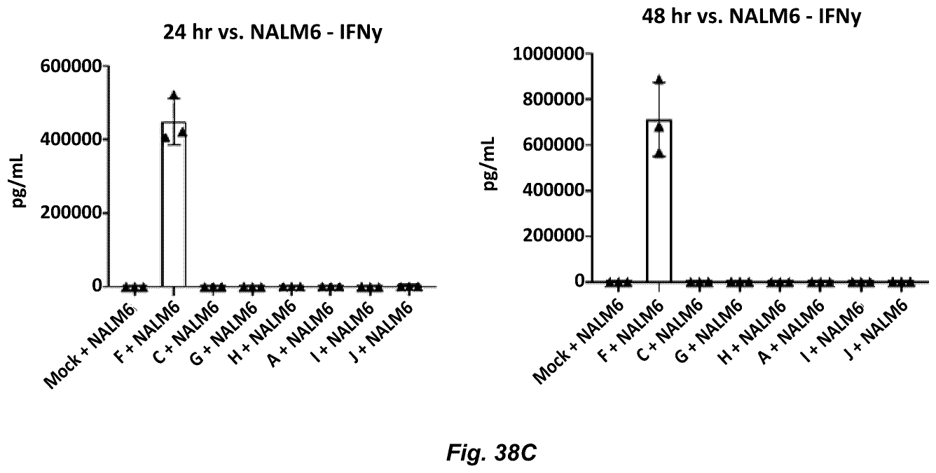

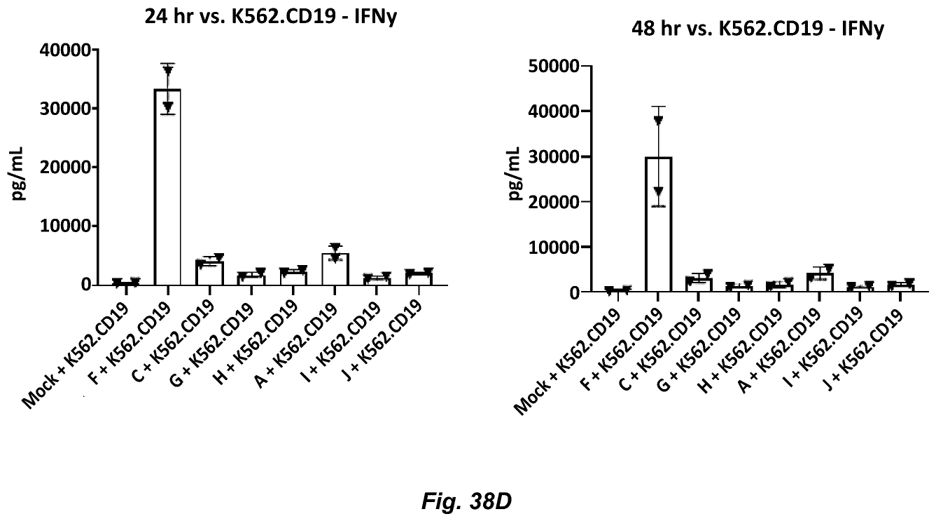

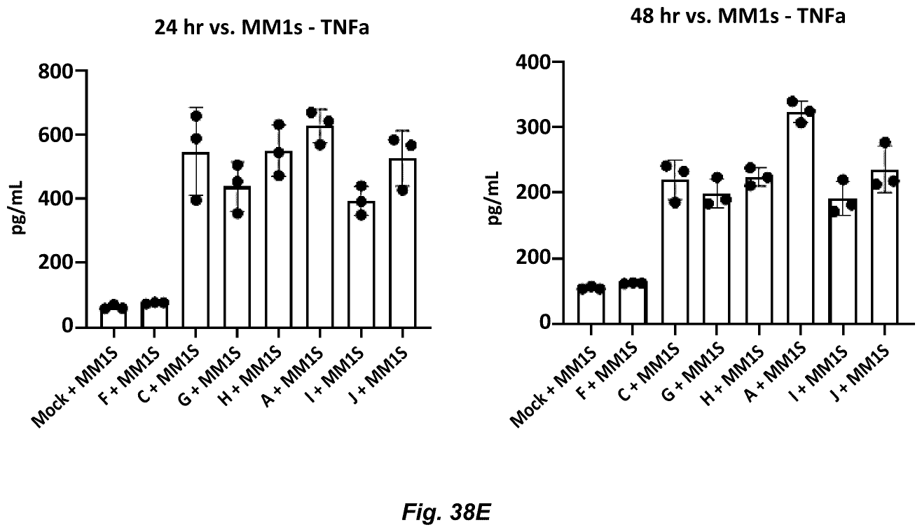

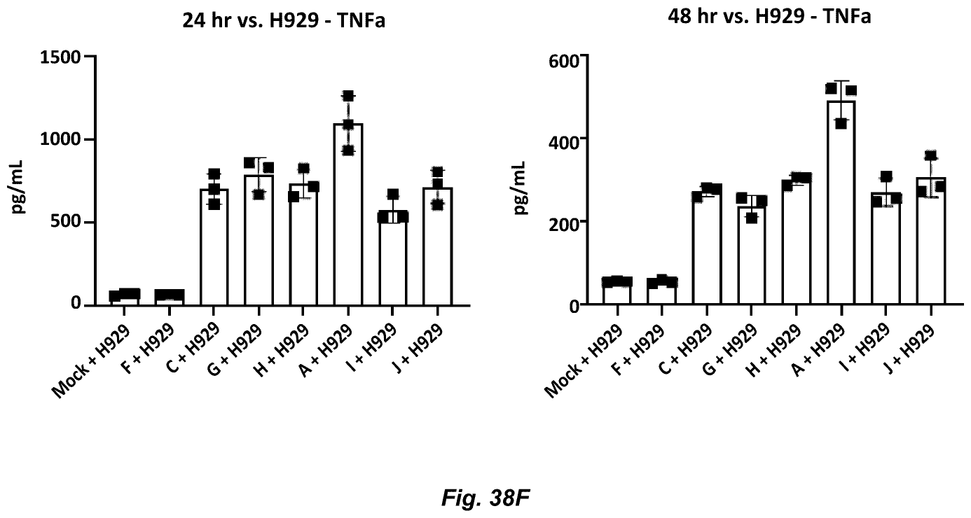

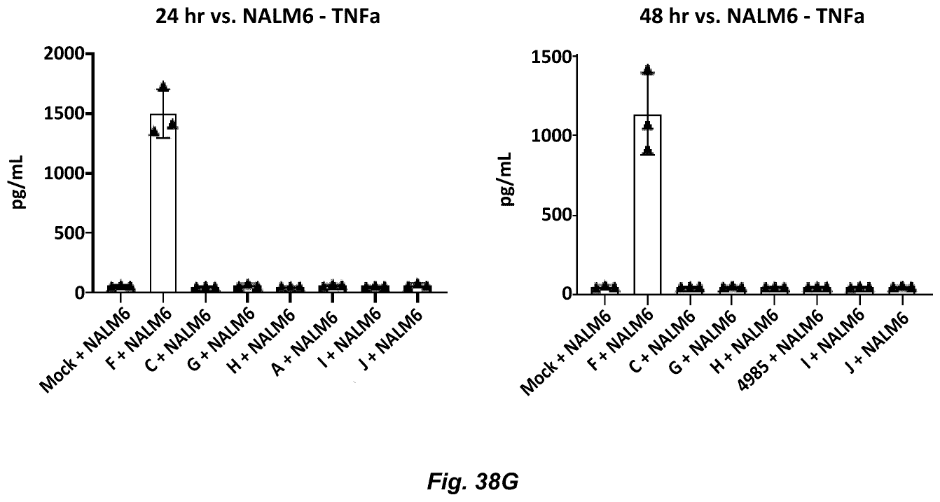

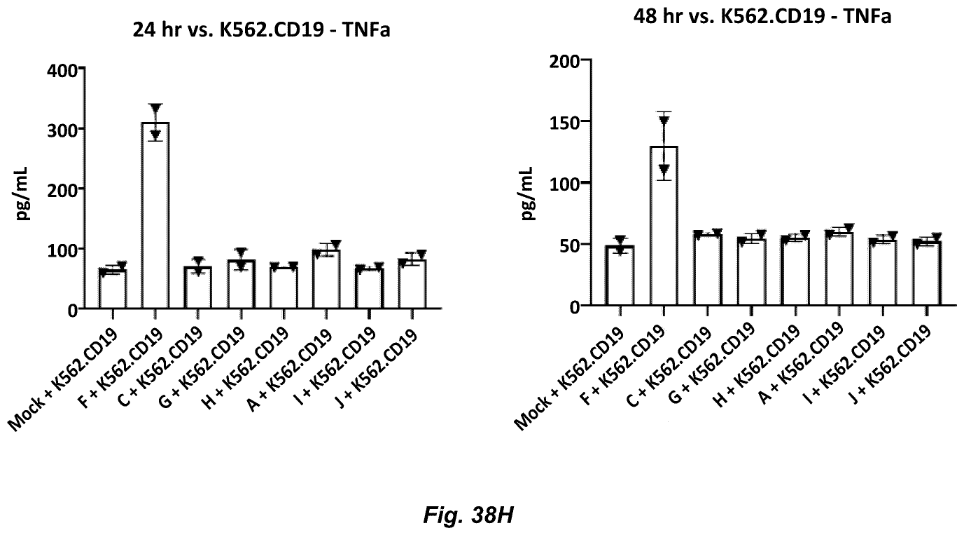

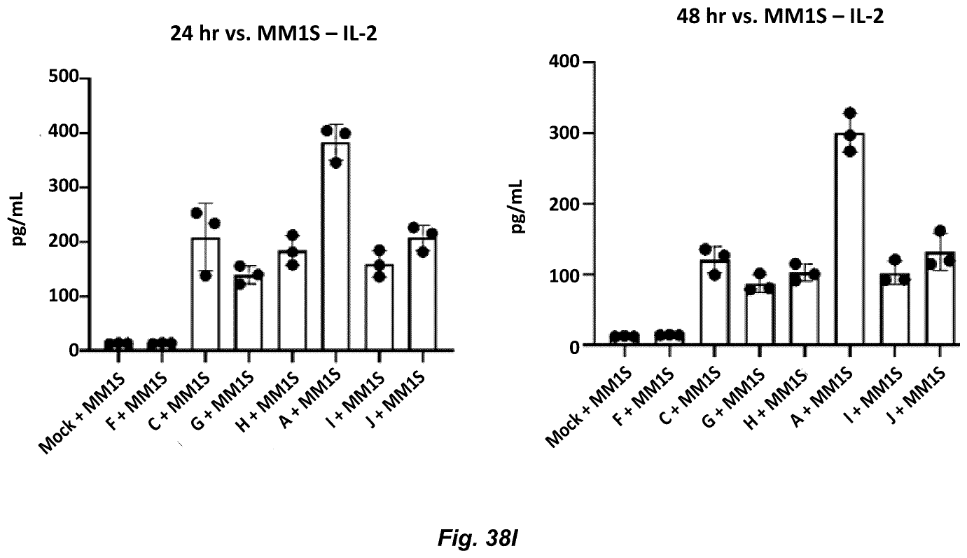

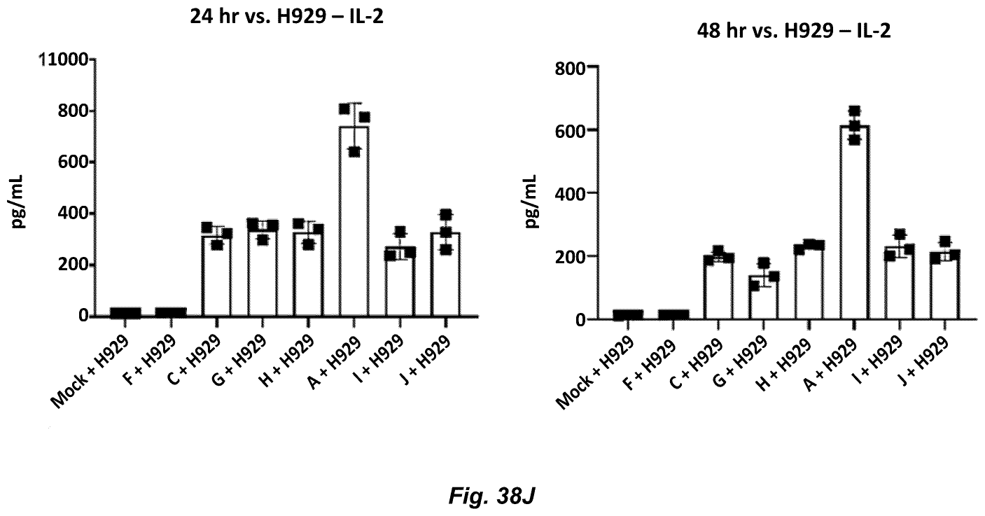

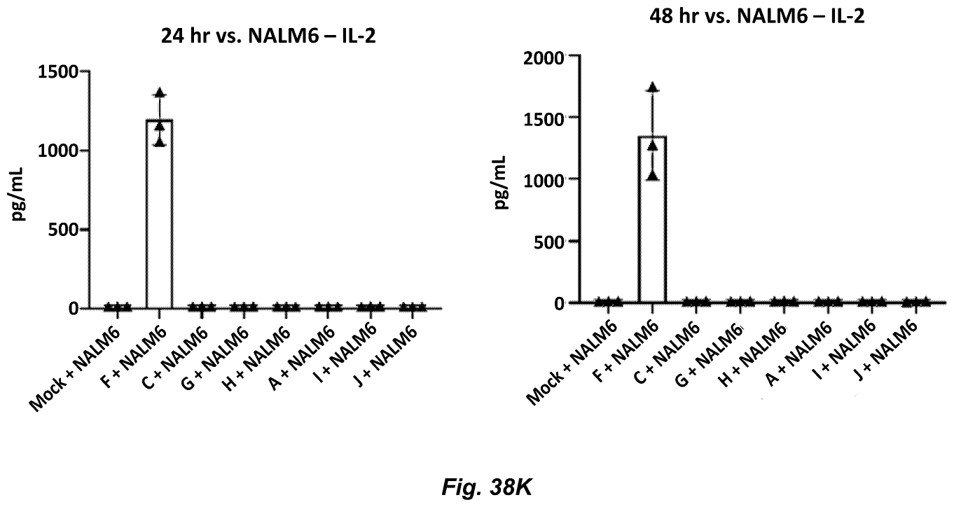

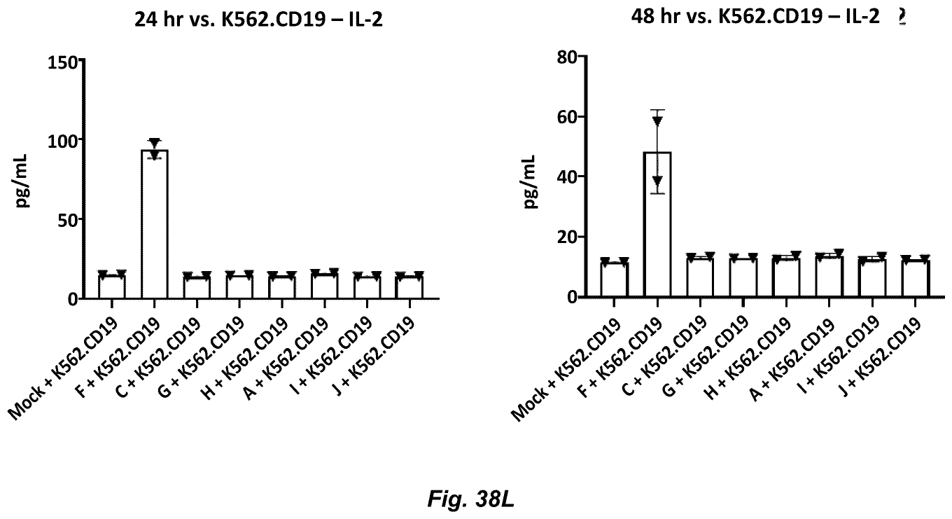

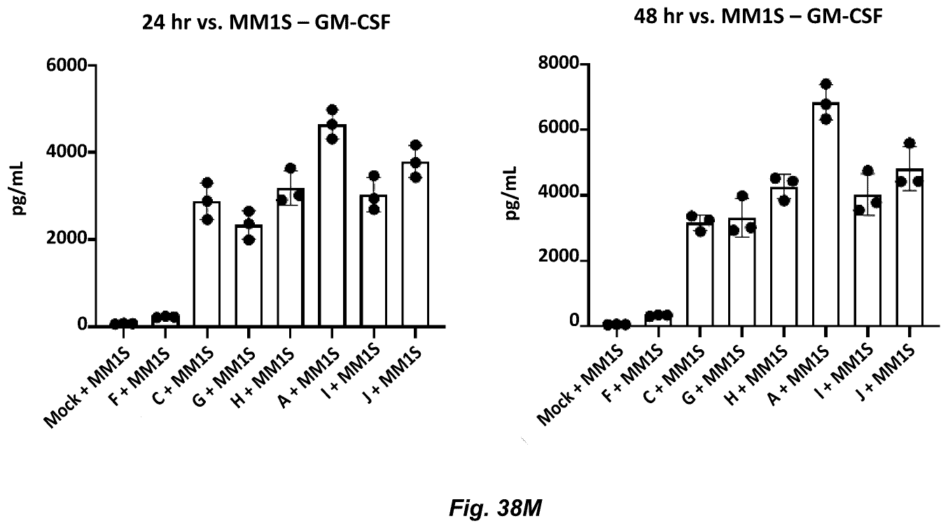

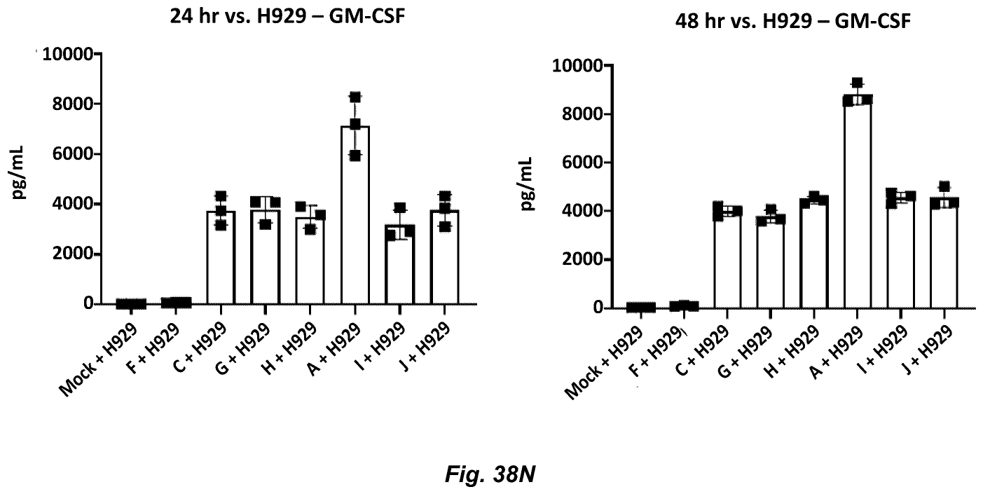

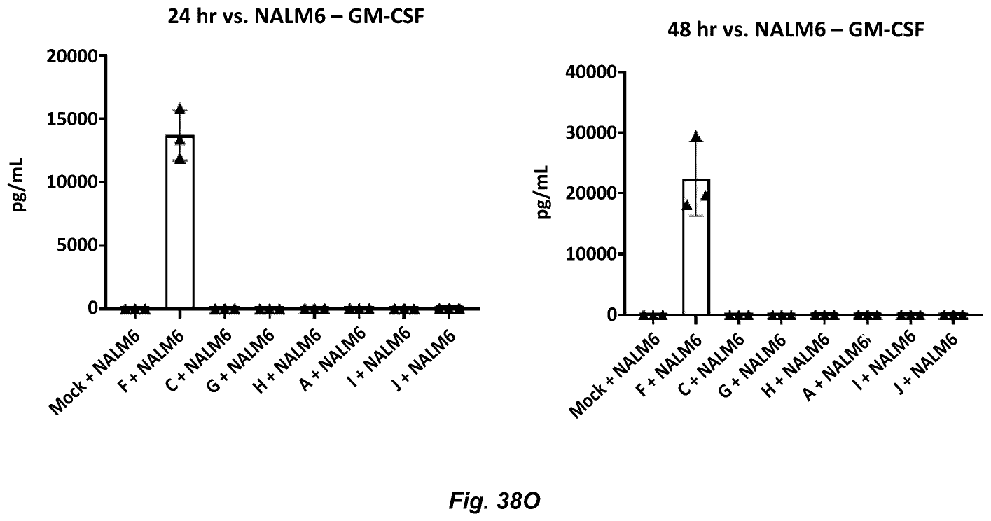

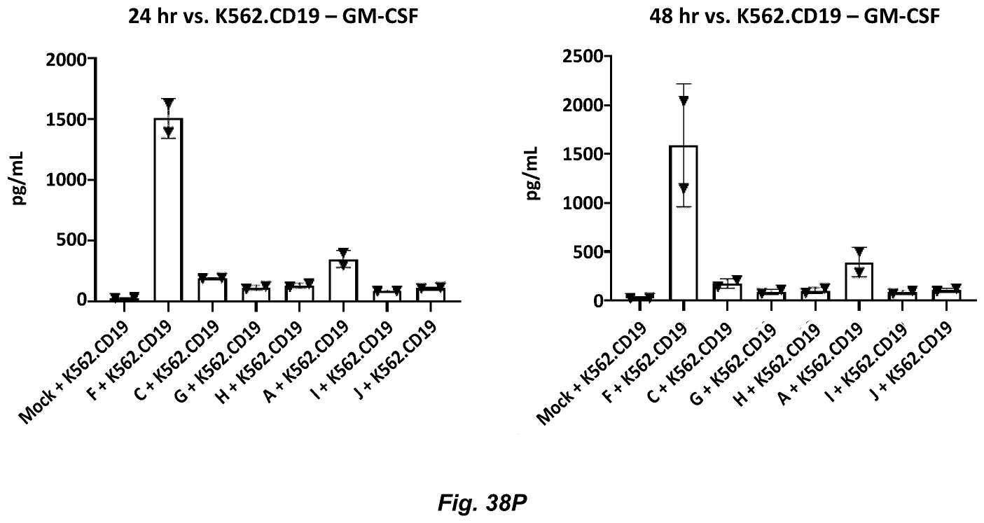

As a follow up, FACS cytotoxicity assays were performed for circular RNAs encoding BCMA-41BBζ, CD19-CD285 or HER2-CD28° C. CAR and an IRES at either 10, 30, 50 or 100 ng per 0.1×10⁶ T cells (FACS imaging for the T cell electroporated with circular RNAs along with their mock counterpart (T cells lacking circular RNAs) was shown in FIGS. 35A and 35B). Circular RNAs were formed from an IVT reaction of DNA templates from Table α1, Table β and Table γ1. The T cells containing circular RNAs (oCAR-T) were co-cultured with one of four target cell-either MMIS (a BCMA positive cell line), NCI-H929 (a BCMA positive cell line) Nalm6 cells (a CD19 positive cell line), a CD19 T stable cell line (e.g., K562.CD19)—at an E:T ratio of 1:1. 24 hours post co-culture of the oCAR-T cells with the MMIS, Nalm6, NCI-H929, or CD19 T stable cells. MMIS+Mock (i.e., MMIS tumor cells cocultured with T cells not electroporated with circular RNAs), MMIS (i.e., MMIS tumor cells not cocultured with T cells), Nalm6+Mock (i.e., Nalm6 tumor cells cocultured with T cells not electroporated with circular RNAs), Nalm6 (i.e., Nalm6 tumor cells not co-cultured with T cells), Mock+NCI-H929 (i.e., NCI-H929 tumor cells cocultured with T cells not electroporated with circular RNAs), and Mock+K562.CD19 (i.e., K562.CD19 tumor cells cocultured with T cells not electroporated with circular RNAs) were used as controls. Resulting FACS imaging for each of the cell types post 24 hours after co-culturing the T cells with the target cells was calculated. From the FACS imaging, percent cell lysis was calculated (e.g., % cell lysis=(1−((% of live Target Cells in Test Sample)/(% of live Target Cells in Control Sample)))×100) and provided in FIG. 34B (FACS cytotoxicity in MMIS) and 34C (FACS cytotoxicity in Nalm6). Percent target killing for MMIS, NCI-H929, Nalm6 and K562.CD19 cocultured with the T cells along with their viabilities were provided in FIG. 36A-36D. INγ (shown in FIGS. 37A and 37B, 38A-38P), IL2, Granzyme and B, GM-CSF, IL2 and TNFα production was evaluated by a commercially available cytokine secretion kit (e.g., MSD) for cytokine secretion levels at either 24 or 48 hours post co-culture of target cell to the T cell.

TABLE γ1

| | Internal Ribosome Entry | | CAR |
|---|---|---|---|
| ID | Site (IRES) Sequence | CAR Sequence | Sequence |
| DNA | GTGGCCACGCCCGGGCCACCGAT | ATGGCTCTGCCTGTGACAGCTCTGC | MALPVTALLL |
| Template K | ACTTCCTTCACTCCTTCGGGAC | TGCTGCCTCTGGCTCTGCTTCTGCA | PLALLLHAAR |
| (Construct | TGTTGGGGAGGAACACAACAGGG | TGCCGCCAGACCTGACATCCAGATG | PDIQMTQSPS |
| K | CTCCCCTGTTTTCCCATTCCTTC | ACTCAGAGCCCCAGCAGCCTGTCTG | SLSASVGDRV |
| | CCCCTTTTCCCAACCCCAACCGC | CCTCTGTGGGAGACAGAGTGACAAT | TITCRASQDV |
| | CGTATCTGGTGGCGGCAAGACAC | TACCTGCCGGGCCAGCCAGGATGTG | NTAVAWYQQK |
| | ACGGGTCTTTCCCTCTAAAGCAC | AATACTGCTGTCGCCTGGTATCAAC | PGKAPKLLIY |
| | AATTGTGTGTGTGTCCCAGGTCC | AAAAGCCTGGCAAGGCCCCTAAGCT | SASFLYSGVP |
| | TCCTGCGTACGGTGCGGGAGTGC | CCTGATCTACAGCGCCAGCTTTCTG | SRFSGSRSGT |
| | TCCCACCCAACTGTTGTAAGCCT | TACAGCGGCGTGCCCAGCAGATTCT | DFTLTISSLQ |
| | GTCCAACGCGTCGTCCTGGCAAG | CCGGAAGCAGAAGCGGCACAGATTT | PEDFATYYCQ |
| | ACTATGACGTCGCATGTTCCGCT | CACACTGACCATAAGCAGCCTGCAG | QHYTTPPTFG |
| | GCGGATGCCGACCGGGTAACCGG | CCAGAGGATTTCGCCACCTACTATT | QGTKVEIKRT |
| | TTCCCCAGTGTGTGTAGTGCGAT | GCCAGCAGCACTACACCACACCTCC | GSTSGSGKPG |
| | CTTCCAGGTCCTCCTGGTTGGCG | AACCTTTGGCCAGGGCACCAAGGTC | SGEGSEVQLV |
| | TTGTCCAGAAACTGCTTCAGGTA | GAGATTAAGAGAACAGGCAGCACAT | ESGGGLVQPG |
| | AGTGGGGTGTGCCCAATCCCTAC | CTGGCTCTGGCAAACCTGGATCTGG | GSLRLSCAAS |
| | AAAGGTTGATTCTTTCACCACCT | CGAGGGCTCTGAAGTCCAGCTGGTG | GFNIKDTYIH |
| | TAGGAATGCTCCGGAGGTACCCC | GAATCTGGCGGAGGACTGGTTCAAC | WVRQAPGKGL |
| | AGCAACAGCTGGGATCTGACCGG | CTGGCGGCTCTCTGAGACTGTCTTG | EWVARIYPTN |
| | AGGCTAATTGTCTACGGGTGGTG | TGCCGCCTCCGGCTTCAACATCAAG | GYTRYADSVK |
| | TTTCCTTTTTCTTTTCACACAAC | GACACCTACATCCACTGGGTCCGAC | GRFTISADTS |
| | TCTACTGCTGACAACTCACTGAC | AAGCCCCAGGCAAAGGACTTGAGTG | KNTAYLQMNS |
| | TATCCACTTGCTCTGTCACG | GGTCGCCAGGATCTACCCCACCAAC | LRAEDTAVYY |
| | (SEQ ID NO: 17) | GGCTACACCAGATACGCCGACTCTG | CSRWGGDGFY |
| | | TGAAGGGCAGATTCACCATCTCTGC | AMDVWGQGTL |
| | | CGACACCAGCAAGAATACCGCCTAC | VTVSSIEVMY |
| | | CTGCAGATGAACTCCCTGAGAGCCG | PPPYLDNEKS |
| | | AAGATACCGCTGTGTATTACTGTTC | NGTIIHVKGK |
| | | CAGATGGGGAGGCGACGGCTTCTAC | HLCPSPLFPG |
| | | GCCATGGATGTTTGGGGCCAAGGCA | PSKPFWVLVV |
| | | CCCTCGTGACCGTTTCTTCTATCGA | VGGVLACYSL |
| | | AGTGATGTACCCTCCACCTTACCTG | LVTVAFIIFW |
| | | GACAACGAGAAGTCCAACGGCACCA | VRSKRSRLLH |
| | | TCATCCACGTGAAGGGCAAGCACCT | SDYMNMTPRR |
| | | GTGTCCTTCTCCACTGTTCCCCGGA | PGPTRKHYQP |
| | | CCTAGCAAGCCTTTCTGGGTGCTCG | YAPPRDFAAY |
| | | TTGTTGTTGGCGGCGTGCTGGCCTG | RSRVKFSRSA |
| | | TTACTCTCTGCTGGTTACCGTGGCC | DAPAYQQGQN |
| | | TTCATCATCTTTTGGGTCCGAAGCA | QLYNELNLGR |
| | | AGCGGAGCCGGCTGCTGCACTCCGA | REEYDVLDKR |
| | | CTACATGAACATGACCCCTAGACGG | RGRDPEMGGK |
| | | CCCGGACCAACCAGAAAGCACTACC | PRRKNPQEGL |
| | | AGCCTTACGCTCCTCCTAGAGACTT | YNELQKDKMA |
| | | CGCCGCCTACCGGTCCAGAGTGAAG | EAYSEIGMKG |
| | | TTCAGCAGATCCGCCGATGCTCCCG | ERRRGKGHDG |
| | | CCTATCAGCAGGGCCAAAACCAGCT | LYQGLSTATK |
| | | GTACAACGAGCTGAACCTGGGGAGA | DTYDALHMQA |
| | | AGAGAAGAGTACGACGTGCTGGACA | LPPR (SEQ |
| | | AGCGGAGAGGCAGAGATCCTGAAAT | ID NO: |
| | | GGGCGGCAAGCCCAGACGGAAGAAT | 117) |
| | | CCTCAAGAGGGCCTGTATAATGAGC | |
| | | TGCAGAAAGACAAGATGGCCGAGGC | |
| | | CTACAGCGAGATCGGAATGAAGGGC | |
| | | GAGCGCAGAAGAGGCAAGGGACACG | |
| | | ATGGACTGTACCAGGGCCTGAGCAC | |
| | | CGCCACCAAGGATACCTATGATGCC | |
| | | CTGCACATGCAGGCCCTGCCTCCAA | |
| | | GA (SEQ ID NO: 101) | |

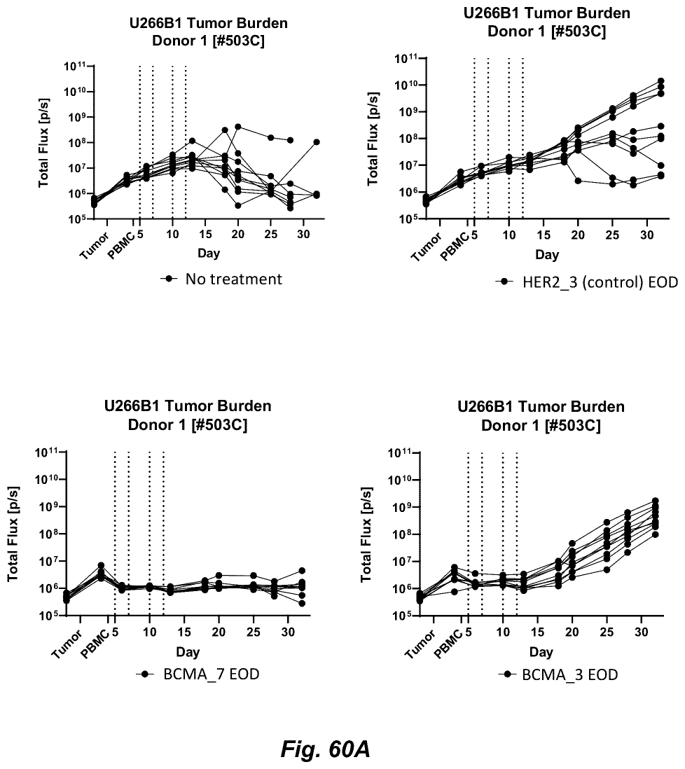

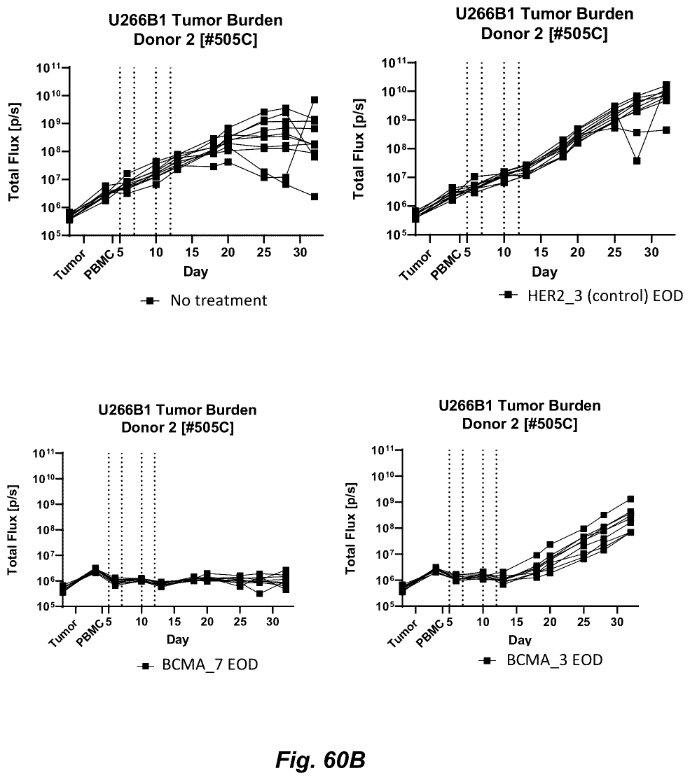

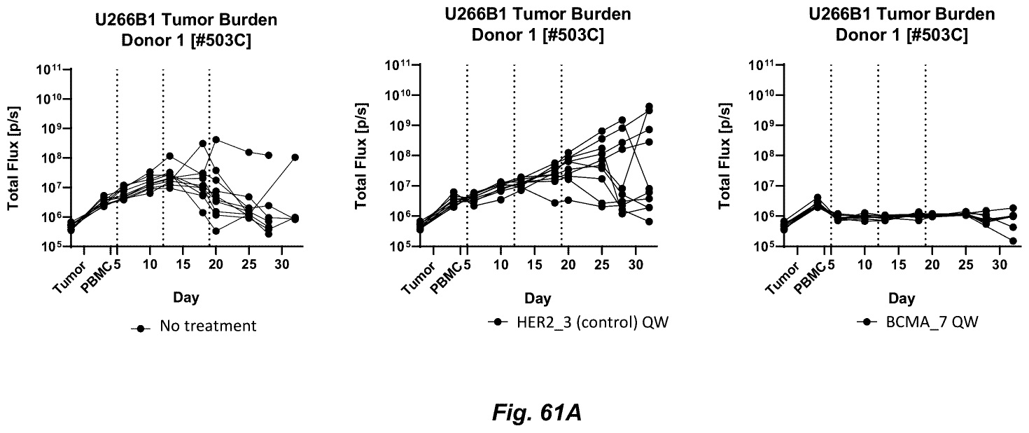

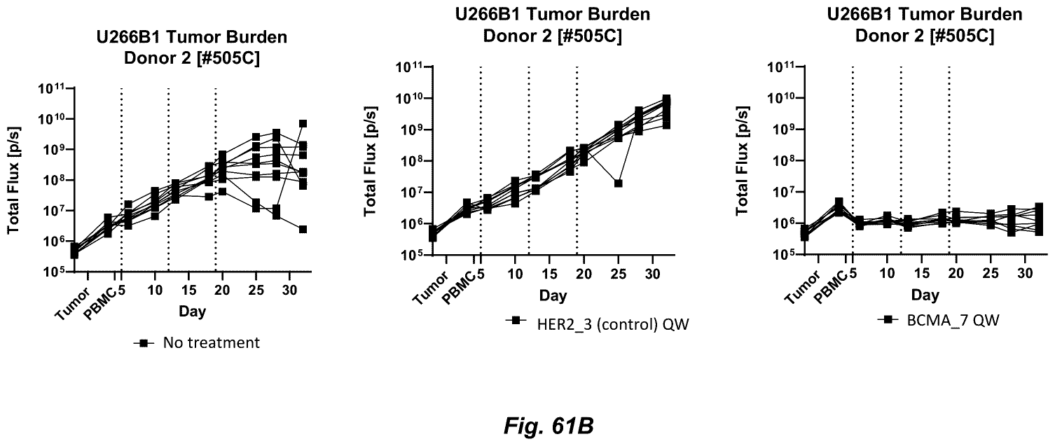

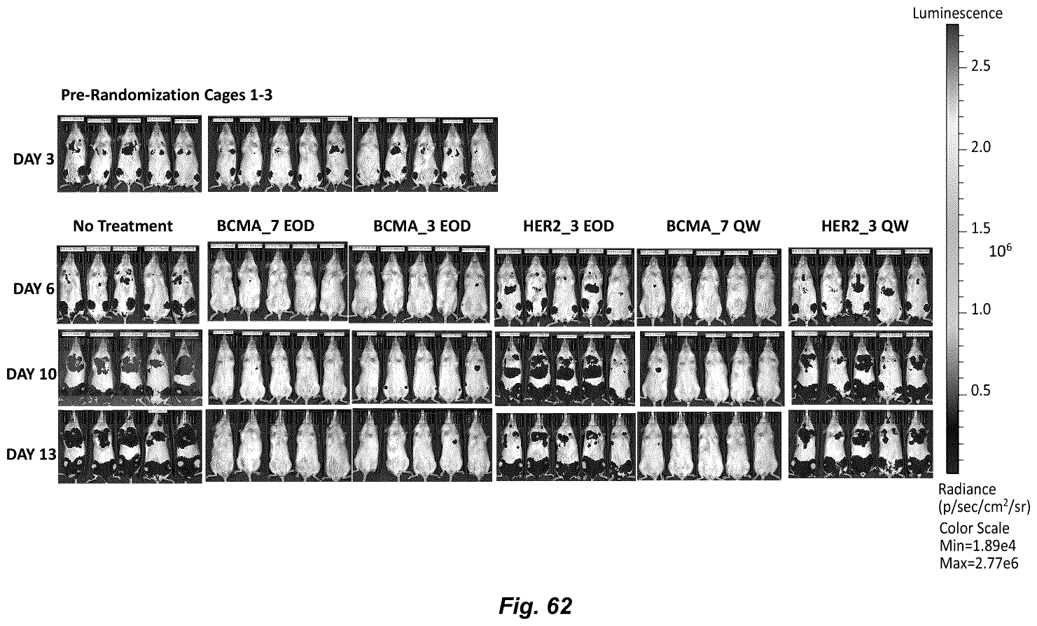

The circular RNA constructs encoding BCMA CAR were tested in vivo. NSG mice were prepared at Day-1. 5M U266 cells were injected at Day 0. 10M PBMC cells (n=2 donors) were injected at Day 4. The circular RNA constructs were injected either at EOD (2 mpk), i.e., Day 8, Day 10, Day 13, and Day 15, or QW (2 mpk), i.e., Day 8, Day 15, and Day 22. Controls were HER2 oCAR-treated mice and untreated mice. Twice weekly, tumor burden was quantified via IVIS as described herein. EOD dosing demonstrated BCMA oCAR-dependent tumor control (FIG. 60). QW dosing demonstrated BCMA oCAR-dependent tumor control (FIG. 61). Exemplary IVIS images show tumor control by BCMA oCAR as compared to control (FIG. 62).

### Example 16: Circular RNAs Encoding HER2 CARs Induction of Cytotoxicity In Vitro

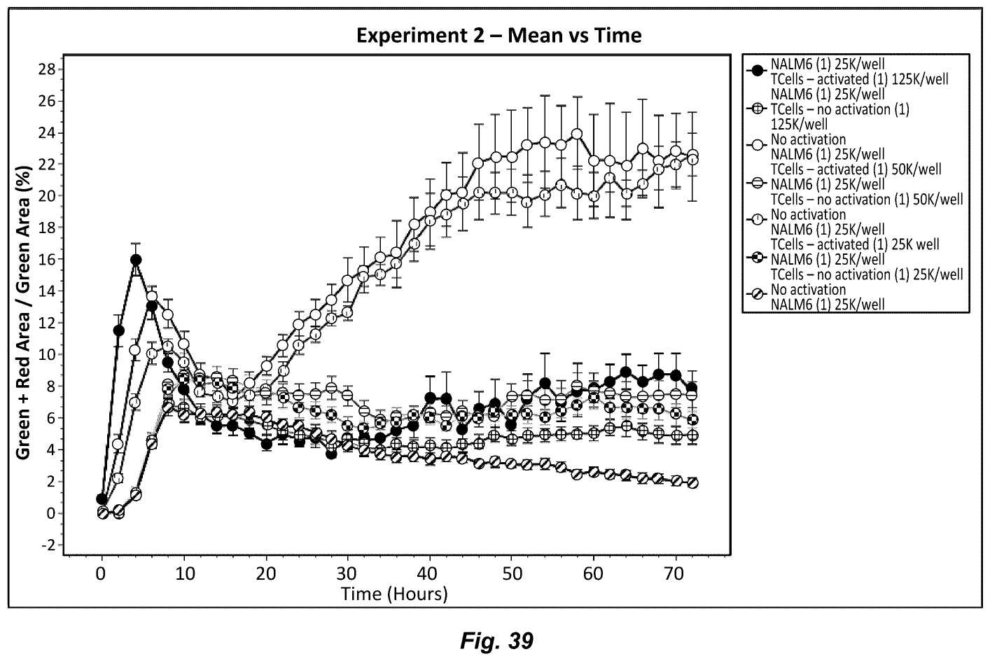

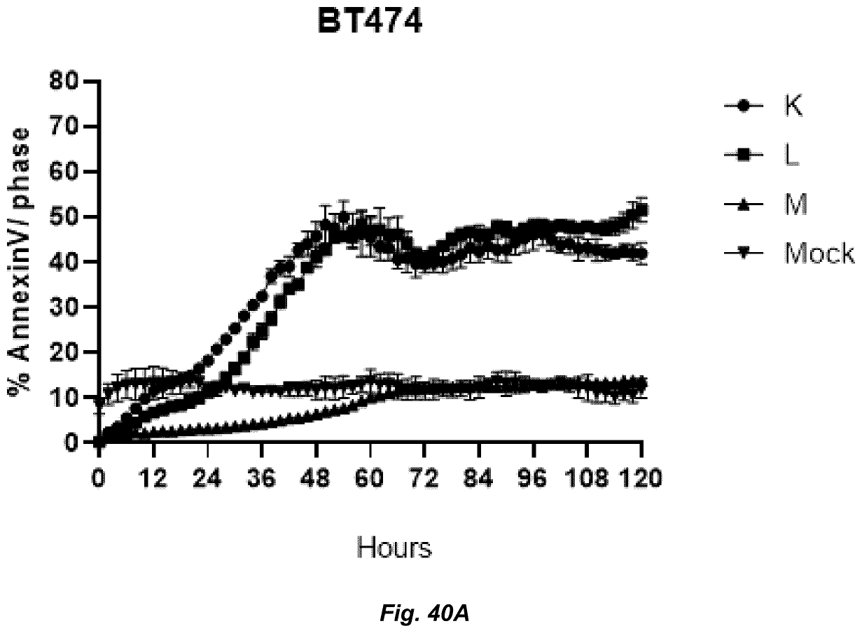

Plates were coated with 0.01% poly-L-omithine solution or 5 μL/mL fibronectin diluted in 0.1% BSA. The dilute apoptosis reagent (e.g., Annexin V) was prepared in a medium and cell treatments are prepared. Cell treatments comprised of circular RNAs comprising a 3′ exon segment, a Caprine Kobuvirus or a Hunnivirus internal ribosome entry site (IRES), a coding region encoding HER2, and a 5′ exon segment with an E:T ratio of 1:1 or 1:2. Circular RNAs were derived from DNA Templates from Table Y1 or Table & that underwent IVT reactions 100 μL/well of 25,000-50,000 of HER2 positive BT474 or SKBR2 target cells were placed into the coated 96-well plates and allowed to adhere overnight. After about 24 hours, the BT474/SKBR3 cells were adhered and the cell treatment at 0 and 100 ng dosages containing the circular RNAs and Annexin V were added to the BT474 or SKBR2 cells. As a control, some BT474/SKBR3 cells were not given any circular RNAs but were given Annexin V. All the plates containing the target cells were analyzed in a live-cell analysis portfolio system (e.g., an IncuCyte) which captured images every 2-3 hours. In the imaging alive Nalm6 cells retained a green fluorescence, and Annexin V apoptotic cells had a red luminescence. % apoptotic cells were then calculated by measuring the amount of ((green area+red area)/green area). Resulting % apoptotic cells was shown in FIG. 39.

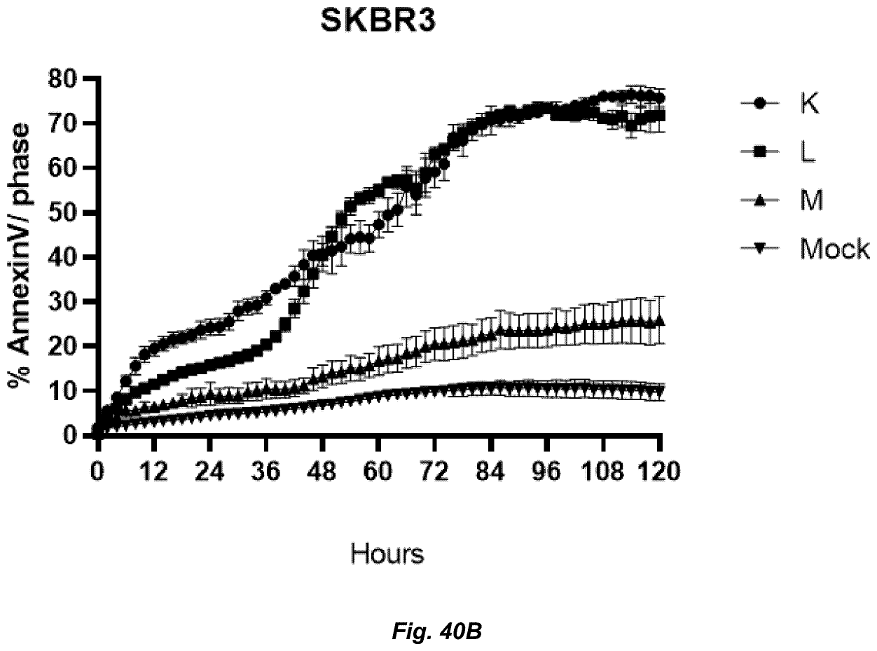

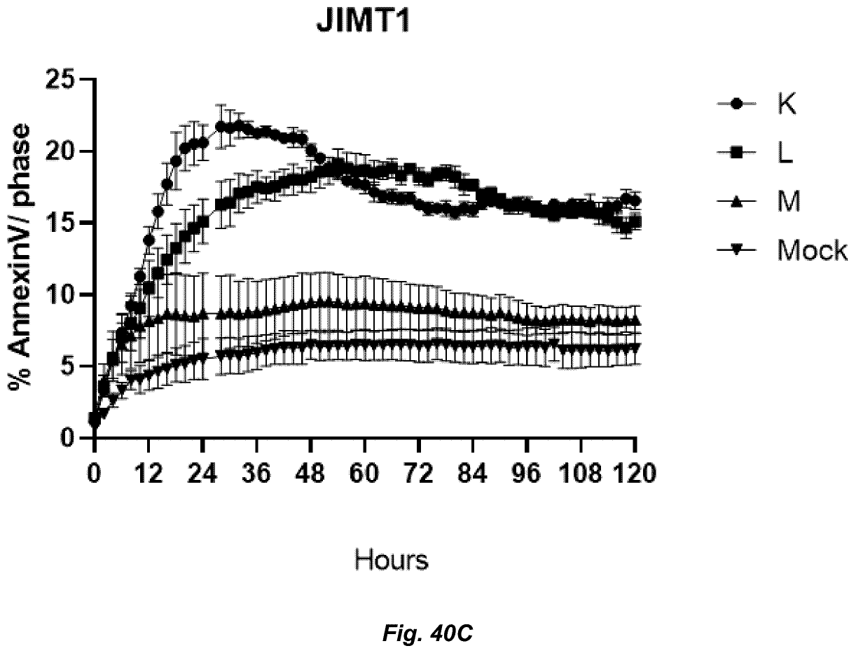

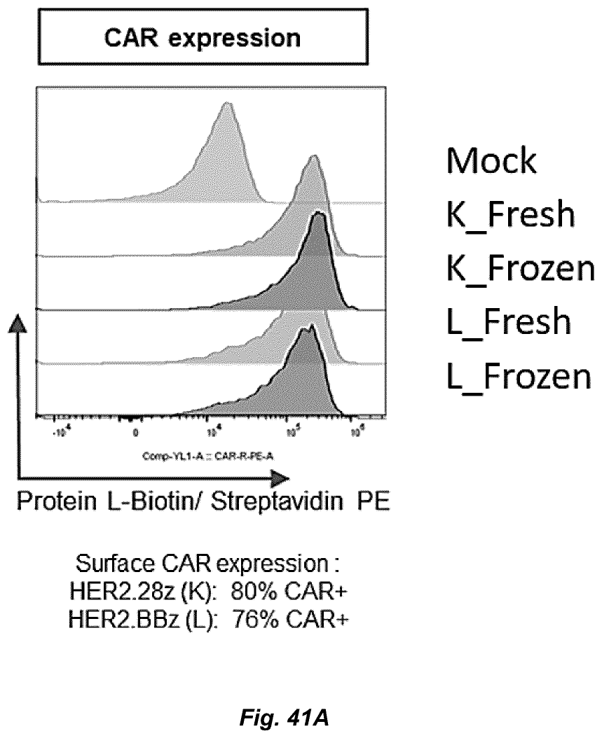

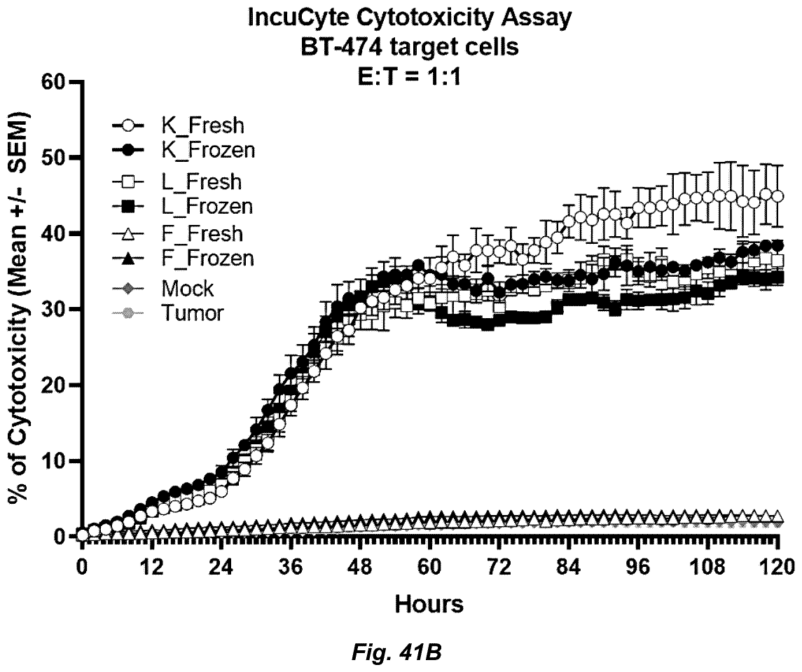

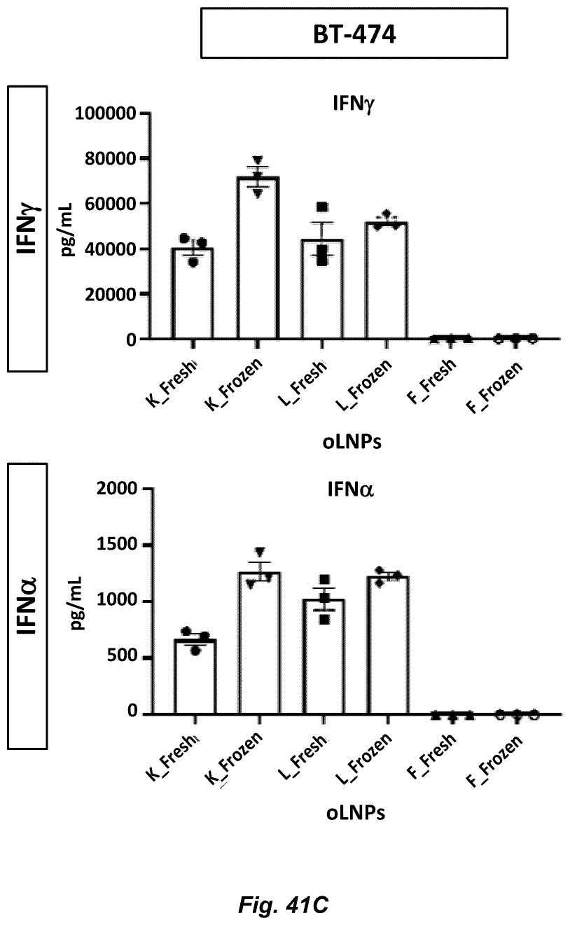

Further, activated PBMC T cells from a single donor were prepared. Separately, circular RNAs encoding HER2-CD28ζ, HER2-41BBζ or CD19-CD28ζ CAR prepared from an IVT reaction of DNA templates comprising a 3′ intron segment, a 3′ exon segment, an internal ribosome entry site (IRES), a coding region encoding either a HER2.28ζ, HER2.BBζ or CD19-28ζ CAR, a 5′ exon segment, and a 5′ intron segment. The circular RNAs were transfected onto the activated PBMC T cells using frozen or fresh lipid nanoparticles comprising an ionizable lipid from Table 3 at a concentration listed in the table below (Table γ2). For comparison purposes, activated PBMC T cells were given no circular RNAs as a control. On the day of post-delivery of the circular RNAs to activated PBMC T cells, the circular RNA-activated PBMC T cells were co-cultured with one of three HER2 positive cell lines (i.e., BT474, SKBR3, JIMT1). The resulting co-cultured cells given Annexin V and were analyzed in live-cell analysis portfolio system (e.g., an IncuCyte). FIGS. 40A-40C depict the results of the live-cell analysis portfolio system as calculated by % Annexin V/phase. FIG. 41A-41B illustrates fresh and frozen LNP delivery of the circular RNAs and FIG. 41C depicts cytokine release (e.g., INFγ and/or TNFα) of the cocultured cells.

TABLE γ2

| | | RNA | | | |
|---|---|---|---|---|---|
| Coding Region of | DNA | Concentration | EE | Diameter | |
| Circular RNA | Template | (μg/mL) | (%) | (nm) | PDI |
| HER2-CD28ζ | K | 494 | 93 | 82 | 0.06 |
| CAR | | | | | |
| HER2-41BBζ | L | 463 | 93 | 80 | 0.06 |
| CAR | | | | | |
| CD19-CD28ζ | M | 498 | 93 | 85 | 0.00 |
| CAR | | | | | |

TABLE 8

| | Internal Ribosome Entry | | CAR |
|---|---|---|---|
| ID | Site (IRES) Sequence | CAR Sequence | Sequence |
| DNA | GTGGCCACGCCCGGGCCACCGAT | ATGGCTCTGCCTGTGACAGCTCTGCT | MALPVTAL |
| Template L | ACTTCCCTTCACTCCTTCGGGAC | GCTGCCTCTGGCTCTGCTTCTGCATG | LLPLALLL |
| (Construct | TGTTGGGGAGGAACACAACAGGG | CCGCCAGACCTGACATCCAGATGACT | HAARPDIQ |
| L) | CTCCCCTGTTTTCCCATTCCTTC | CAGAGCCCCAGCAGCCTGTCTGCCTC | MTQSPSSL |
| | CCCCTTTTCCCAACCCCAACCGC | TGTGGGAGACAGAGTGACAATTACCT | SASVGDRV |
| | CGTATCTGGTGGCGGCAAGACAC | GCCGGGCCAGCCAGGATGTGAATACT | TITCRASQ |
| | ACGGGTCTTTCCCTCTAAAGCAC | GCTGTCGCCTGGTATCAACAAAAGCC | DVNTAVAW |
| | AATTGTGTGTGTGTCCCAGGTCC | TGGCAAGGCCCCTAAGCTCCTGATCT | YQQKPGKA |
| | TCCTGCGTACGGTGCGGGAGTGC | ACAGCGCCAGCTTTCTGTACAGCGGC | PKLLIYSA |
| | TCCCACCCAACTGTTGTAAGCCT | GTGCCCAGCAGATTCTCCGGAAGCAG | SFLYSGVP |
| | GTCCAACGCGTCGTCCTGGCAAG | AAGCGGCACAGATTTCACACTGACCA | SRFSGSRS |
| | ACTATGACGTCGCATGTTCCGCT | TAAGCAGCCTGCAGCCAGAGGATTTC | GTDFTLTI |
| | GCGGATGCCGACCGGGTAACCGG | GCCACCTACTATTGCCAGCAGCACTA | SSLQPEDF |
| | TTCCCCAGTGTGTGTAGTGCGAT | CACCACACCTCCAACCTTTGGCCAGG | ATYYCQQH |
| | CTTCCAGGTCCTCCTGGTTGGCG | GCACCAAGGTCGAGATTAAGAGAACA | YTTPPTFG |
| | TTGTCCAGAAACTGCTTCAGGTA | GGCAGCACATCTGGCTCTGGCAAACC | QGTKVEIK |
| | AGTGGGGTGTGCCCAATCCCTAC | TGGATCTGGCGAGGGCTCTGAAGTCC | RTGSTSGS |
| | AAAGGTTGATTCTTTCACCACCT | AGCTGGTGGAATCTGGCGGAGGACTG | GKPGSGEG |
| | TAGGAATGCTCCGGAGGTACCCC | GTTCAACCTGGCGGCTCTCTGAGACT | SEVQLVES |
| | AGCAACAGCTGGGATCTGACCGG | GTCTTGTGCCGCCTCCGGCTTCAACA | GGGLVQPG |
| | AGGCTAATTGTCTACGGGTGGTG | TCAAGGACACCTACATCCACTGGGTC | GSLRISCA |
| | TTTCCTTTTTCTTTTCACACAAC | CGACAAGCCCCAGGCAAAGGACTTGA | ASGENIKD |
| | TCTACTGCTGACAACTCACTGAC | GTGGGTCGCCAGGATCTACCCCACCA | TYIHWVRQ |
| | TATCCACTTGCTCTGTCACG | ACGGCTACACCAGATACGCCGACTCT | APGKGLEW |
| | (SEQ ID NO: 17) | GTGAAGGGCAGATTCACCATCTCTGC | VARIYPTN |
| | | CGACACCAGCAAGAATACCGCCTACC | GYTRYADS |
| | | TGCAGATGAACTCCCTGAGAGCCGAA | VKGRFTIS |
| | | GATACCGCTGTGTATTACTGTTCCAG | ADTSKNTA |
| | | ATGGGGAGGCGACGGCTTCTACGCCA | YLQMNSLR |
| | | TGGATGTTTGGGGCCAAGGCACCCTC | AEDTAVYY |
| | | GTGACCGTTTCTTCTACCACCACACC | CSRWGGDG |
| | | AGCTCCTCGGCCTCCAACTCCTGCTC | FYAMDVWG |
| | | CTACAATTGCCAGCCAGCCTCTGTCT | QGTLVTVS |
| | | CTGAGGCCCGAAGCTTGTAGACCTGC | STTTPAPR |
| | | TGCTGGCGGAGCCGTGCATACAAGAG | PPTPAPTI |
| | | GACTGGATTTCGCCTGCGACATCTAC | ASQPLSLR |
| | | ATCTGGGCTCCTCTGGCCGGAACATG | PEACRPAA |
| | | TGGCGTTCTGCTGCTGAGCCTGGTCA | GGAVHTRG |
| | | TCACCCTGTACTGTAAGCGGGGCAGA | LDFACDIY |
| | | AAGAAGCTGCTGTACATCTTCAAGCA | IWAPLAGT |
| | | GCCCTTCATGCGGCCCGTGCAGACCA | CGVLLLSL |
| | | CACAAGAGGAAGATGGCTGCTCCTGC | VITLYCKR |
| | | AGATTCCCCGAGGAAGAAGAAGGCGG | GRKKLLYI |
| | | CTGCGAGCTGAGAGTGAAGTTCAGCA | FKQPFMRP |
| | | GATCCGCCGATGCTCCCGCCTATCAG | VQTTQEED |
| | | CAGGGCCAAAACCAGCTGTACAACGA | GCSCREPE |
| | | GCTGAACCTGGGGAGAAGAGAAGAGT | EEEGGCEL |
| | | ACGACGTGCTGGACAAGCGGAGAGGC | RVKFSRSA |
| | | AGAGATCCTGAAATGGGGGGCAAGCC | DAPAYQQG |
| | | CAGACGGAAGAATCCTCAAGAGGGCC | QNQLYNEL |
| | | TGTATAATGAGCTGCAGAAAGACAAG | NLGRREEY |
| | | ATGGCCGAGGCCTACAGCGAGATCGG | DVLDKRRG |
| | | AATGAAGGGCGAGCGCAGAAGAGGCA | RDPEMGGK |
| | | AGGGACACGATGGACTGTACCAGGGC | PRRKNPQE |
| | | CTGAGCACCGCCACCAAGGATACCTA | GLYNELQK |
| | | TGATGCCCTGCACATGCAGGCCCTGC | DKMAEAYS |
| | | CTCCAAGA (SEQ ID NO: 102) | EIGMKGER |
| | | | RRGKGHDG |
| | | | LYQGLSTA |
| | | | TKDTYDAL |
| | | | HMQALPPR |
| | | | (SEQ ID |
| | | | NO: 118) |
| DNA | CCCCCCTCCCCCCCTTCCCTTCC | ATGGCACTGCCCGTCACCGCACTCCT | MALPVTAL |
| Template M | CTTTGCAACGCAACAATTGTAAG | GCTCCCACTGGCACTGCTGCTCCATG | LLPLALLL |
| (Construct | TGCCCTCACCTGTCAATTGGGAC | CAGCTCGCCCCGATATCCAGATGACC | HAARPDIQ |
| M) | CACCACTTTCAGTGACCCCATGC | CAGACCACCTCTAGCCTCAGCGCCTC | MTQTTSSL |
| | GAAGTGCTGAGAGAAAGGAAGCT | TCTGGGTGACCGCGTCACCATCTCTT | SASLGDRV |
| | TTCTTACCCTTCATTTGTGAACC | GCCGGGCCAGCCAAGACATCTCTAAG | TISCRASQ |
| | CACTGGTCTAAGCCGCTTGGAAT | TACCTGAACTGGTACCAGCAGAAACC | DISKYLNW |
| | ACGATGAGTGGAAAAGTTCATTC | TGACGGAACCGTGAAGCTGCTGATCT | YQQKPDGT |
| | TTAATGGAGTGAAACATGCTTAA | ACCACACCAGTCGGCTGCATTCCGGG | VKLLIYHT |
| | ATTTCCAGCTCGTGCTGGTCTTT | GTGCCTTCCAGGTTCAGCGGTTCCGG | SRLHSGVP |
| | CCAGTACGGGGCGGCCCTGTCTG | CTCTGGGACCGATTATAGTCTCACCA | SRFSGSGS |
| | GCCGTAATTCTTCAGAGTGTCAC | TCTCCAACCTCGAGCAGGAGGACATC | GTDYSLTI |
| | GCCACACTTGTGGATCTCACGTG | GCAACCTACTTCTGCCAGCAGGGGAA | SNLEQEDI |
| | CCACATGACAGCGCTACAGCTGG | CACCCTGCCCTACACCTTCGGTGGCG | ATYFCQQG |
| | AACTGGGTGCTTGGTGCCCATGG | GGACCAAGCTGGAGATCACTGGAGGT | NTLPYTFG |
| | AGTAACAGCGAAAAGTGTTAGAT | GGTGGCAGCGGAGGTGGAGGATCAGG | GGTKLEIT |
| | CAAGCCTTGCTTGGGCTATGAGC | TGGAGGCGGTAGCGAGGTGAAGCTGC | GGGGSGGG |
| | CTGCGGAACAACAACTGGTAACA | AGGAGTCCGGACCTGGTCTGGTGGCC | GSGGGGSE |
| | GTTGCCTCAGGGGCCGAAAGCCA | CCAAGCCAGTCCCTCAGCGTCACCTG | VKLQESGP |
| | CGGTGTTAACAGCACCCTCATAG | CACAGTGTCCGGGGTGTCCCTGCCTG | GLVAPSQS |
| | TTTGATCCACCTCAGGGTGGTGA | ACTACGGTGTCTCCTGGATCAGGCAA | LSVTCTVS |
| | TGTTTAGCAGTTAGTAGTTGCCA | CCACCCCGGAAGGGTCTCGAGTGGCT | GVSLPDYG |
| | ATCTGTGTTCACTGAAATCTCGG | GGGCGTCATCTGGGGCTCCGAGACCA | VSWIRQPP |
| | CATACCGTGTAGTGTACAGGGGT | CCTACTACAACAGCGCTCTGAAGTCC | RKGLEWLG |
| | GAAGGATGCCCAGAAGGTACCCG | CGGCTGACCATCATCAAAGACAACTC | VIWGSETT |
| | TAGGTAACCTTAAGAGACTATGG | CAAGAGCCAGGTGTTCTTGAAGATGA | YYNSALKS |
| | ATCTGATCTGGGGCCTTGTCCGG | ACTCCCTGCAAACCGATGACACCGCC | RLTIIKDN |
| | AGTGCTTTACACACGGCTCAAGG | ATCTACTACTGCGCCAAGCACTACTA | SKSQVELK |
| | TTAAAAAACGTCTAGCCCCACAG | CTATGGCGGTAGCTACGCCATGGATT | MNSLQTDD |
| | AGCCCGAGGGATTCGGGTTTTCC | ATTGGGGTCAGGGCACCAGTGTCACC | TAIYYCAK |
| | CTTTAAAAACCCGACTAGAGCTT | GTCTCCTCCATCGAGGTGATGTACCC | HYYYGGSY |
| | ATGGTGACAATTATTGCTGTTCA | TCCACCCTATCTGGACAACGAGAAGT | AMDYWGQG |
| | GACGAACAGTGTAATTGTTGTCT | CCAACGGCACCATCATCCACGTGAAG | TSVTVSSI |
| | ATTCACAGCAGTTCTATCAGAGC | GGCAAGCACCTGTGCCCTAGCCCTCT | EVMYPPPY |
| | TTTTCCCACAACGGATCTTCTTG | GTTCCCAGGACCCTCCAAGCCCTTCT | LDNEKSNG |
| | GCAAGCAAATACAGCAGGAGTCA | GGGTGCTGGTCGTGGTGGGAGGAGTC | TIIHVKGK |
| | AT (SEQ ID NO: 8) | CTGGCCTGCTATTCCCTCCTCGTCAC | HLCPSPLF |
| | | CGTGGCATTTATCATCTTCTGGGTCC | PGPSKPFW |
| | | GGAGCAAGCGGTCACGCCTGCTCCAC | VLVVVGGV |
| | | TCCGACTACATGAACATGACTCCTCG | LACYSLLV |
| | | CAGACCTGGACCCACCCGGAAGCACT | TVAFIIFW |
| | | ACCAGCCTTATGCCCCACCCCGCGAC | VRSKRSRL |
| | | TTTGCCGCTTACCGCTCTCGGGTCAA | LHSDYMNM |
| | | GTTCTCTCGGTCAGCAGACGCCCCTG | TPRRPGPT |
| | | CATACCAGCAGGGCCAGAACCAGCTG | RKHYQPYA |
| | | TATAACGAGCTGAACCTCGGCAGACG | PPRDFAAY |
| | | GGAGGAGTACGATGTGCTGGACAAGA | RSRVKFSR |
| | | GGAGAGGCAGAGACCCCGAGATGGGT | SADAPAYQ |
| | | GGTAAGCCACGGCGCAAGAACCCACA | QGQNQLYN |
| | | GGAGGGCTTGTACAACGAACTGCAGA | ELNLGRRE |
| | | AGGACAAGATGGCCGAGGCCTACAGC | EYDVLDKR |
| | | GAGATCGGCATGAAGGGAGAGAGGCG | RGRDPEMG |
| | | CAGGGGCAAGGGTCACGACGGCCTGT | GKPRRKNP |
| | | ACCAAGGGCTGTCCACCGCAACCAAG | QEGLYNEL |
| | | GACACCTACGATGCCCTGCACATGCA | QKDKMAEA |
| | | GGCCCTCCCACCAAGG (SEQ ID | YSEIGMKG |
| | | NO: 19) | ERRRGKGH |
| | | | DGLYQGLS |
| | | | TATKDTYD |
| | | | ALHMQALP |
| | | | PR (SEQ |
| | | | ID NO: |
| | | | 29) |
| DNA | ATTCTCGGGCTACGGCCCTGGAG | ATGGCTCTGCCTGTGACAGCTCTGCT | MALPVTAL |
| Template N | CCACTCCGGCTCCTAAAGATTTA | GCTGCCTCTGGCTCTGCTTCTGCATG | LLPLALLL |
| (Construct | GAAGTTTGAGCACACCCGCCCAC | CCGCCAGACCTGACATCCAGATGACT | HAARPDIQ |
| N) | TAGGGCCCCCCATCCAGGGGGGC | CAGAGCCCCAGCAGCCTGTCTGCCTC | MTQSPSSL |
| | AACGGGCAAGCACTTCTGTTTCC | TGTGGGAGACAGAGTGACAATTACCT | SASVGDRV |
| | CCGGTATGATCTGATAGGCTGTA | GCCGGGCCAGCCAGGATGTGAATACT | TITCRASQ |
| | ACCACGGCTGAAACAGAGATTAT | GCTGTCGCCTGGTATCAACAAAAGCC | DVNTAVAW |
| | CGTTATCCGCTTCACTACTTCGA | TGGCAAGGCCCCTAAGCTCCTGATCT | YQQKPGKA |
| | GAAGCCTAGTAATGATGGGTGAA | ACAGCGCCAGCTTTCTGTACAGCGGC | PKLLIYSA |
| | ATTGAATCCGTTGATCCGGTGTC | GTGCCCAGCAGATTCTCCGGAAGCAG | SFLYSGVP |
| | TCCCCCACACCAGAAACTCATGA | AAGCGGCACAGATTTCACACTGACCA | SRFSGSRS |
| | TGAGGGTTGCCATCCCGGCTACG | TAAGCAGCCTGCAGCCAGAGGATTTC | GTDFTLTI |
| | GCGACGTAGCGGGCATCCCTGCG | GCCACCTACTATTGCCAGCAGCACTA | SSLQPEDE |
| | CTGGCATGAGGCCTCTTAGGAGG | CACCACACCTCCAACCTTTGGCCAGG | ATYYCQQH |
| | ACGGATGATATGGATCTTGTCGT | GCACCAAGGTCGAGATTAAGAGAACA | YTTPPTFG |
| | GAAGAGCCTATTGAGCTAGTGTC | GGCAGCACATCTGGCTCTGGCAAACC | QGTKVEIK |
| | GACTCCTCCGCCCCCGTGAATGC | TGGATCTGGCGAGGGCTCTGAAGTCC | RTGSTSGS |
| | GGCTAATCCTAACCCCGGAGCAG | AGCTGGTGGAATCTGGCGGAGGACTG | GKPGSGEG |
| | GTGGGTCCAATCCAGGGCCTGGC | GTTCAACCTGGCGGCTCTCTGAGACT | SEVQLVES |
| | CTGTCGTAATGCGTAAGTCTGGG | GTCTTGTGCCGCCTCCGGCTTCAACA | GGGLVQPG |
| | ACGGAACCGACTACTTTCGGGAA | TCAAGGACACCTACATCCACTGGGTC | GSLRLSCA |
| | GGCGTGTTTCCATTTGTTCATTA | CGACAAGCCCCAGGCAAAGGACTTGA | ASGENIKD |
| | TTTGTGTGTTTATGGTGACAACT | GTGGGTCGCCAGGATCTACCCCACCA | TYIHWVRQ |
| | CTGGGTAAACGTTCTATTGCGTT | ACGGCTACACCAGATACGCCGACTCT | APGKGLEW |
| | TATTGAGAGATTCCCAACAATTG | GTGAAGGGCAGATTCACCATCTCTGC | VARIYPTN |
| | AACAAACGAGAACTACCTGTTTT | CGACACCAGCAAGAATACCGCCTACC | GYTRYADS |
| | ATTAAATTTACACAGAGAAGAAT | TGCAGATGAACTCCCTGAGAGCCGAA | VKGRFTIS |
| | TACA (SEQ ID NO: 16) | GATACCGCTGTGTATTACTGTTCCAG | ADTSKNTA |
| | | ATGGGGAGGCGACGGCTTCTACGCCA | YLQMNSLR |
| | | TGGATGTTTGGGGCCAAGGCACCCTC | AEDTAVYY |
| | | GTGACCGTTTCTTCTATCGAAGTGAT | CSRWGGDG |
| | | GTACCCTCCACCTTACCTGGACAACG | FYAMDVWG |
| | | AGAAGTCCAACGGCACCATCATCCAC | QGTLVTVS |
| | | GTGAAGGGCAAGCACCTGTGTCCTTC | SIEVMYPP |
| | | TCCACTGTTCCCCGGACCTAGCAAGC | PYLDNEKS |
| | | CTTTCTGGGTGCTCGTTGTTGTTGGC | NGTIIHVK |
| | | GGCGTGCTGGCCTGTTACTCTCTGCT | GKHLCPSP |
| | | GGTTACCGTGGCCTTCATCATCTTTT | LFPGPSKP |
| | | GGGTCCGAAGCAAGCGGAGCCGGCTG | FWVLVVVG |
| | | CTGCACTCCGACTACATGAACATGAC | GVLACYSL |
| | | CCCTAGACGGCCCGGACCAACCAGAA | LVTVAFII |
| | | AGCACTACCAGCCTTACGCTCCTCCT | FWVRSKRS |
| | | AGAGACTTCGCCGCCTACCGGTCCAG | RLLHSDYM |
| | | AGTGAAGTTCAGCAGATCCGCCGATG | NMTPRRPG |
| | | CTCCCGCCTATCAGCAGGGCCAAAAC | PTRKHYQP |
| | | CAGCTGTACAACGAGCTGAACCTGGG | YAPPRDFA |
| | | GAGAAGAGAAGAGTACGACGTGCTGG | AYRSRVKF |
| | | ACAAGCGGAGAGGCAGAGATCCTGAA | SRSADAPA |
| | | ATGGGGGGCAAGCCCAGACGGAAGAA | YQQGQNQL |
| | | TCCTCAAGAGGGCCTGTATAATGAGC | YNELNLGR |
| | | TGCAGAAAGACAAGATGGCCGAGGCC | REEYDVLD |
| | | TACAGCGAGATCGGAATGAAGGGCGA | KRRGRDPE |
| | | GCGCAGAAGAGGCAAGGGACACGATG | MGGKPRRK |
| | | GACTGTACCAGGGCCTGAGCACCGCC | NPQEGLYN |
| | | ACCAAGGATACCTATGATGCCCTGCA | ELQKDKMA |
| | | CATGCAGGCCCTGCCTCCAAGA (SEQ | EAYSEIGM |
| | | ID NO: 101) | KGERRRGK |
| | | | GHDGLYQG |
| | | | LSTATKDT |
| | | | YDALHMQA |
| | | | LPPR |
| | | | (SEQ ID |
| | | | NO: 117) |
| DNA | CCCCCCTCCCCCCCTTCCCTTCC | ATGGCTCTGCCTGTGACAGCTCTGCT | MALPVTAL |
| Template O | CTTTGCAACGCAACAATTGTAAG | GCTGCCTCTGGCTCTGCTTCTGCATG | LLPLALLL |
| (Construct | TGCCCTCACCTGTCAATTGGGAC | CCGCCAGACCTGACATCCAGATGACT | HAARPDIQ |
| O) | CACCACTTTCAGTGACCCCATGC | CAGAGCCCCAGCAGCCTGTCTGCCTC | MTQSPSSL |
| | GAAGTGCTGAGAGAAAGGAAGCT | TGTGGGAGACAGAGTGACAATTACCT | SASVGDRV |
| | TTCTTACCCTTCATTTGTGAACC | GCCGGGCCAGCCAGGATGTGAATACT | TITCRASQ |
| | CACTGGTCTAAGCCGCTTGGAAT | GCTGTCGCCTGGTATCAACAAAAGCC | DVNTAVAW |
| | ACGATGAGTGGAAAAGTTCATTC | TGGCAAGGCCCCTAAGCTCCTGATCT | YQQKPGKA |
| | TTAATGGAGTGAAACATGCTTAA | ACAGCGCCAGCTTTCTGTACAGCGGC | PKLLIYSA |
| | ATTTCCAGCTCGTGCTGGTCTTT | GTGCCCAGCAGATTCTCCGGAAGCAG | SFLYSGVP |
| | CCAGTACGGGGCGGCCCTGTCTG | AAGCGGCACAGATTTCACACTGACCA | SRFSGSRS |
| | GCCGTAATTCTTCAGAGTGTCAC | TAAGCAGCCTGCAGCCAGAGGATTTC | GTDFTLTI |
| | GCCACACTTGTGGATCTCACGTG | GCCACCTACTATTGCCAGCAGCACTA | SSLQPEDF |
| | CCACATGACAGCGCTACAGCTGG | CACCACACCTCCAACCTTTGGCCAGG | ATYYCQQH |
| | AACTGGGTGCTTGGTGCCCATGG | GCACCAAGGTCGAGATTAAGAGAACA | YTTPPTFG |
| | AGTAACAGCGAAAAGTGTTAGAT | GGCAGCACATCTGGCTCTGGCAAACC | QGTKVEIK |
| | CAAGCCTTGCTTGGGCTATGAGC | TGGATCTGGCGAGGGCTCTGAAGTCC | RTGSTSGS |
| | CTGCGGAACAACAACTGGTAACA | AGCTGGTGGAATCTGGCGGAGGACTG | GKPGSGEG |
| | GTTGCCTCAGGGGCCGAAAGCCA | GTTCAACCTGGCGGCTCTCTGAGACT | SEVQLVES |
| | CGGTGTTAACAGCACCCTCATAG | GTCTTGTGCCGCCTCCGGCTTCAACA | GGGLVQPG |
| | TTTGATCCACCTCAGGGTGGTGA | TCAAGGACACCIACATCCACTGGGTC | GSLRISCA |
| | TGTTTAGCAGTTAGTAGTTGCCA | CGACAAGCCCCAGGCAAAGGACTTGA | ASGFNIKD |
| | ATCTGTGTTCACTGAAATCTCGG | GTGGGTCGCCAGGATCTACCCCACCA | TYIHWVRQ |
| | CATACCGTGTAGTGTACAGGGGT | ACGGCTACACCAGATACGCCGACTCT | APGKGLEW |
| | GAAGGATGCCCAGAAGGTACCCG | GTGAAGGGCAGATTCACCATCTCTGC | VARIYPTN |
| | TAGGTAACCTTAAGAGACTATGG | CGACACCAGCAAGAATACCGCCTACC | GYTRYADS |
| | ATCTGATCTGGGGCCTTGTCCGG | TGCAGATGAACTCCCTGAGAGCCGAA | VKGRFTIS |
| | AGTGCTTTACACACGGCTCAAGG | GATACCGCTGTGTATTACTGTTCCAG | ADTSKNTA |
| | TTAAAAAACGTCTAGCCCCACAG | ATGGGGAGGCGACGGCTTCTACGCCA | YLQMNSLR |
| | AGCCCGAGGGATTCGGGTTTTCC | TGGATGTTTGGGGCCAAGGCACCCTC | AEDTAVYY |
| | CTTTAAAAACCCGACTAGAGCTT | GTGACCGTTTCTTCTATCGAAGTGAT | CSRWGGDG |
| | ATGGTGACAATTATTGCTGTTCA | GTACCCTCCACCTTACCTGGACAACG | FYAMDVWG |
| | GACGAACAGTGTAATTGTTGTCT | AGAAGTCCAACGGCACCATCATCCAC | QGTLVTVS |
| | ATTCACAGCAGTTCTATCAGAGC | GTGAAGGGCAAGCACCTGTGTCCTTC | SIEVMYPP |
| | TTTTCCCACAACGGATCTTCTTG | TCCACTGTTCCCCGGACCTAGCAAGC | PYLDNEKS |
| | GCAAGCAAATACAGCAGGAGTCA | CTTTCTGGGTGCTCGTTGTTGTTGGC | NGTIIHVK |
| | AT (SEQ ID NO: 8) | GGCGTGCTGGCCTGTTACTCTCTGCT | GKHLCPSP |
| | | GGTTACCGTGGCCTTCATCATCTTTT | LFPGPSKP |
| | | GGGTCCGAAGCAAGCGGAGCCGGCTG | FWVLVVVG |
| | | CTGCACTCCGACTACATGAACATGAC | GVLACYSL |
| | | CCCTAGACGGCCCGGACCAACCAGAA | LVTVAFII |
| | | AGCACTACCAGCCTTACGCTCCTCCT | FWVRSKRS |
| | | AGAGACTTCGCCGCCTACCGGTCCAG | RLLHSDYM |
| | | AGTGAAGTTCAGCAGATCCGCCGATG | NMTPRRPG |
| | | CTCCCGCCTATCAGCAGGGCCAAAAC | PTRKHYQP |
| | | CAGCTGTACAACGAGCTGAACCTGGG | YAPPRDFA |
| | | GAGAAGAGAAGAGTACGACGTGCTGG | AYRSRVKF |
| | | ACAAGCGGAGAGGCAGAGATCCTGAA | SRSADAPA |
| | | ATGGGGGGCAAGCCCAGACGGAAGAA | YQQGQNQL |
| | | TCCTCAAGAGGGCCTGTATAATGAGC | YNELNLGR |
| | | TGCAGAAAGACAAGATGGCCGAGGCC | REEYDVLD |
| | | TACAGCGAGATCGGAATGAAGGGCGA | KRRGRDPE |
| | | GCGCAGAAGAGGCAAGGGACACGATG | MGGKPRRK |
| | | GACTGTACCAGGGCCTGAGCACCGCC | NPQEGLYN |
| | | ACCAAGGATACCTATGATGCCCTGCA | ELQKDKMA |
| | | CATGCAGGCCCTGCCTCCAAGA (SEQ | EAYSEIGM |
| | | ID NO: 101) | KGERRRGK |
| | | | GHDGLYQG |
| | | | LSTATKDT |
| | | | YDALHMQA |
| | | | LPPR |
| | | | (SEQ ID |
| | | | NO: 117) |
| DNA | TTTGCTCAGCGTAACTTCTCCGG | ATGGCTCTGCCTGTGACAGCTCTGCT | MALPVTAL |
| Template P | GTTACGTGGAGACCAAAAGGCTA | GCTGCCTCTGGCTCTGCTTCTGCATG | LLPLALLL |
| (Construct | CGGAGACTCGGGCTACGGCCCTG | CCGCCAGACCTGACATCCAGATGACT | HAARPDIQ |
| P) | GAGCACCTAGGTGCTCCTAAAGA | CAGAGCCCCAGCAGCCTGTCTGCCTC | MTQSPSSL |
| | CGTTAGAAGTTGTACAAACTCGC | TGTGGGAGACAGAGTGACAATTACCT | SASVGDRV |
| | CCAATAGGGCCCCCCAACCAGGG | GCCGGGCCAGCCAGGATGTGAATACT | TITCRASQ |
| | GGGTAGCGGGCAAGCACTTCTGT | GCTGTCGCCTGGTATCAACAAAAGCC | DVNTAVAW |
| | TTCCCCGGTATGATCTCATAGGC | TGGCAAGGCCCCTAAGCTCCTGATCT | YQQKPGKA |
| | TGTACCCACGGCTGAAAGAGAGA | ACAGCGCCAGCTTTCTGTACAGCGGC | PKLLIYSA |
| | TTATCGTTACCCGCCTCACTACT | GTGCCCAGCAGATTCTCCGGAAGCAG | SFLYSGVP |
| | TCGAGAAGCCCAGTAATGGTTCA | AAGCGGCACAGATTTCACACTGACCA | SRFSGSRS |
| | TGAAGTTGATCTCGTTGACCCGG | TAAGCAGCCTGCAGCCAGAGGATTTC | GTDFTLTI |
| | TGTTTCCCCCACACCAGAAACCT | GCCACCTACTATTGCCAGCAGCACTA | SSLQPEDE |
| | GTGATGGGGGTGGTCATCCCGGT | CACCACACCTCCAACCTTTGGCCAGG | ATYYCQQH |
| | CATGGCGACATGACGGACCTCCC | GCACCAAGGTCGAGATTAAGAGAACA | YTTPPTFG |
| | CGCGCCGGCACAGGGCCTCTTCG | GGCAGCACATCTGGCTCTGGCAAACC | QGTKVEIK |
| | GAGGACGAGTGACATGGATTCAA | TGGATCTGGCGAGGGCTCTGAAGTCC | RTGSTSGS |
| | CCGTGAAGAGCCTATTGAGCTAG | AGCTGGTGGAATCTGGCGGAGGACTG | GKPGSGEG |
| | TGTTGATTCCTCCGCCCCCGTGA | GTTCAACCTGGCGGCTCTCTGAGACT | SEVQLVES |
| | ATGCGGCTAATCCCAACTCCGGA | GTCTTGTGCCGCCTCCGGCTTCAACA | GGGLVQPG |
| | GCAGGCGGGCCCAAACCAGGGTC | TCAAGGACACCTACATCCACTGGGTC | GSLRLSCA |
| | TGGCCTGTCGTAACGCGAAAGTC | CGACAAGCCCCAGGCAAAGGACTTGA | ASGFNIKD |
| | TGGAGCGGAACCGACTACTTTCG | GTGGGTCGCCAGGATCTACCCCACCA | TYIHWVRQ |
| | GGAAGGCGTGTTTCCTTTTGTTC | ACGGCTACACCAGATACGCCGACTCT | APGKGLEW |
| | CTTTTATCAAGTTTTATGGTGAC | GTGAAGGGCAGATTCACCATCTCTGC | VARIYPTN |
| | AACTCCTGGTAGACGTTTTATTG | CGACACCAGCAAGAATACCGCCTACC | GYTRYADS |
| | CGTTTATTGAGAGATTTCCAACA | TGCAGATGAACTCCCTGAGAGCCGAA | VKGRFTIS |
| | ATTGAACAGACTAGAACCACTTG | GATACCGCTGTGTATTACTGTTCCAG | ADTSKNTA |
| | TTTTATCAAACCCTCACAGAATA | ATGGGGAGGCGACGGCTTCTACGCCA | YLQMNSLR |
| | AGATAACA (SEQ ID NO: 18) | TGGATGTTTGGGGCCAAGGCACCCTC | AEDTAVYY |
| | | GTGACCGTTTCTTCTATCGAAGTGAT | CSRWGGDG |
| | | GTACCCTCCACCTTACCTGGACAACG | FYAMDVWG |
| | | AGAAGTCCAACGGCACCATCATCCAC | QGTLVTVS |
| | | GTGAAGGGCAAGCACCTGTGTCCTTC | SIEVMYPP |
| | | TCCACTGTTCCCCGGACCTAGCAAGC | PYLDNEKS |
| | | CTTTCTGGGTGCTCGTTGTTGTTGGC | NGTIIHVK |
| | | GGCGTGCTGGCCTGTTACTCTCTGCT | GKHLCPSP |
| | | GGTTACCGTGGCCTTCATCATCTTTT | LFPGPSKP |
| | | GGGTCCGAAGCAAGCGGAGCCGGCTG | FWVLVVVG |
| | | CTGCACTCCGACTACATGAACATGAC | GVLACYSL |
| | | CCCTAGACGGCCCGGACCAACCAGAA | LVTVAFII |
| | | AGCACTACCAGCCTTACGCTCCTCCT | FWVRSKRS |
| | | AGAGACTTCGCCGCCTACCGGTCCAG | RLLHSDYM |
| | | AGTGAAGTTCAGCAGATCCGCCGATG | NMTPRRPG |
| | | CTCCCGCCTATCAGCAGGGCCAAAAC | PTRKHYQP |
| | | CAGCTGTACAACGAGCTGAACCTGGG | YAPPRDFA |
| | | GAGAAGAGAAGAGTACGACGTGCTGG | AYRSRVKF |
| | | ACAAGCGGAGAGGCAGAGATCCTGAA | SRSADAPA |
| | | ATGGGCGGCAAGCCCAGACGGAAGAA | YQQGQNQL |
| | | TCCTCAAGAGGGCCTGTATAATGAGC | YNELNLGR |
| | | TGCAGAAAGACAAGATGGCCGAGGCC | REEYDVLD |
| | | TACAGCGAGATCGGAATGAAGGGCGA | KRRGRDPE |
| | | GCGCAGAAGAGGCAAGGGACACGATG | MGGKPRRK |
| | | GACTGTACCAGGGCCTGAGCACCGCC | NPQEGLYN |
| | | ACCAAGGATACCTATGATGCCCTGCA | ELQKDKMA |
| | | CATGCAGGCCCTGCCTCCAAGA (SEQ | EAYSEIGM |
| | | ID NO: 101) | KGERRRGK |
| | | | GHDGLYQG |
| | | | LSTATKDT |
| | | | YDALHMQA |
| | | | LPPR |
| | | | (SEQ ID |
| | | | NO: 117) |

### Example 17: Circular RNAs Encoding HER2 CARs Tumor Killing Activity In Vivo

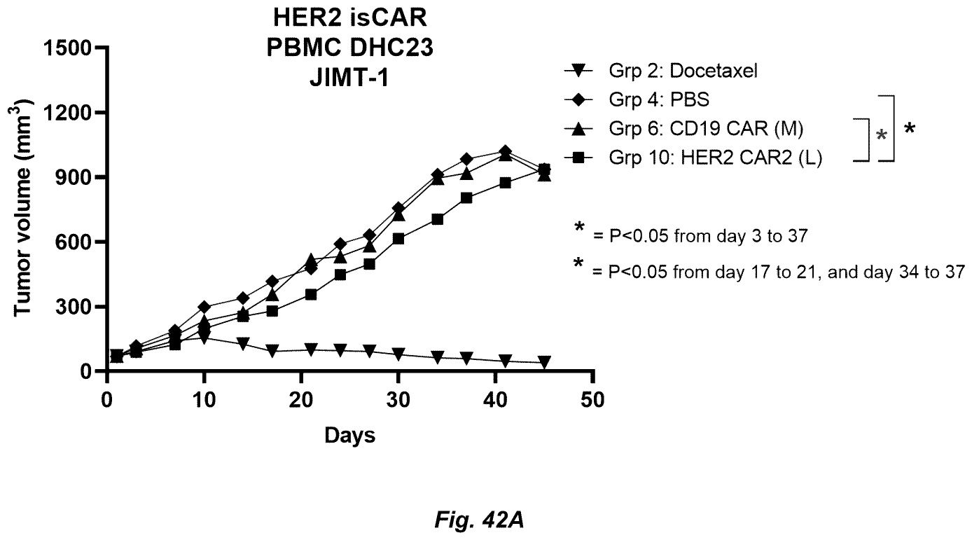

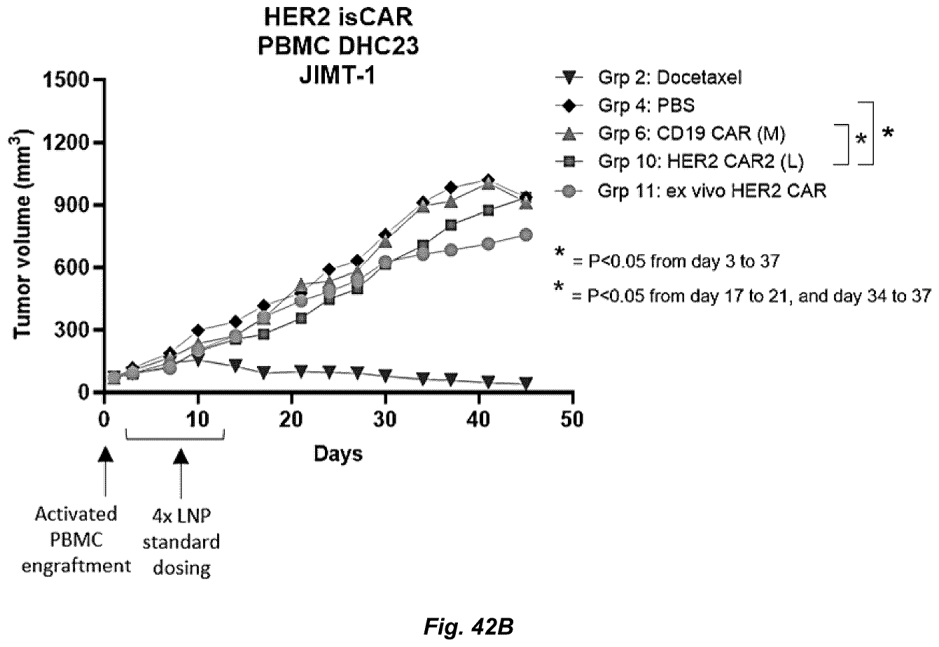

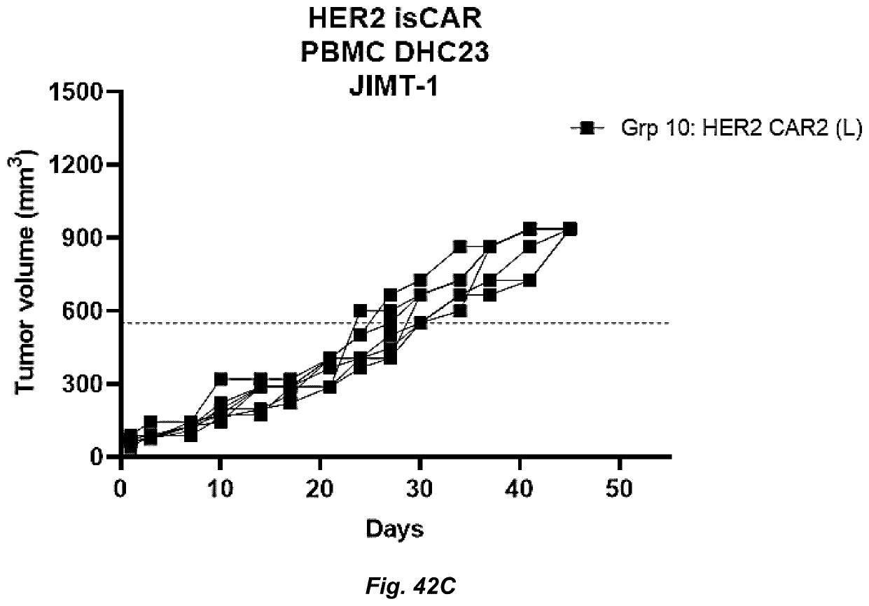

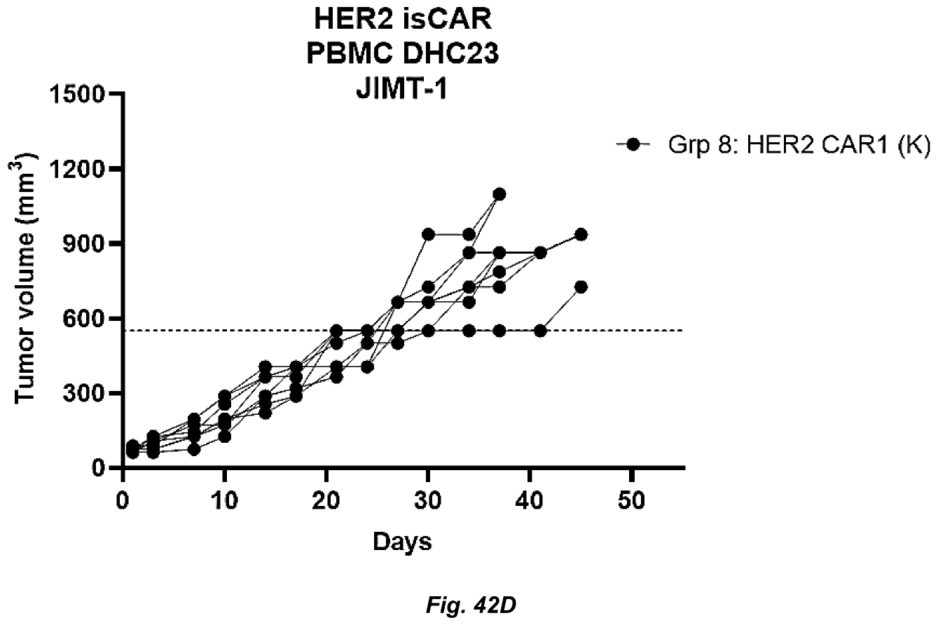

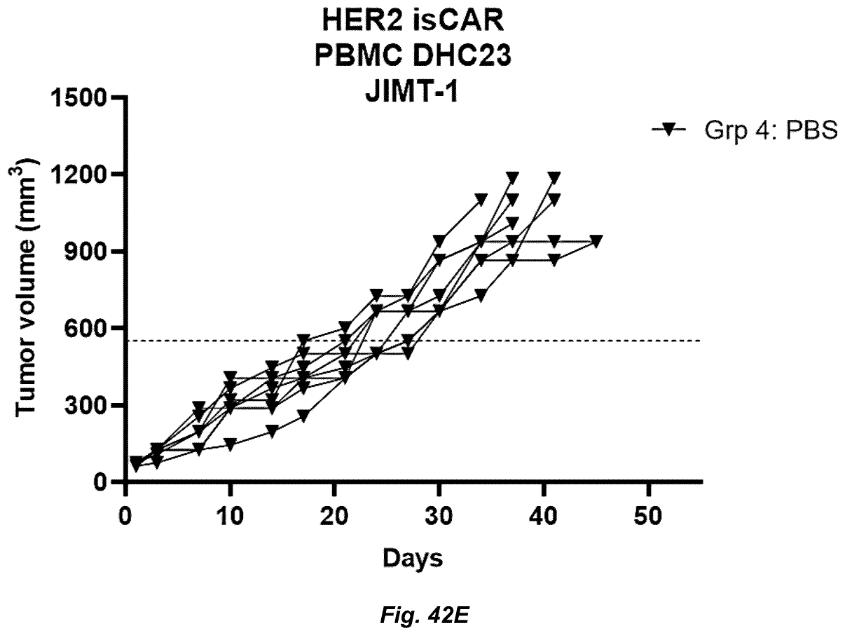

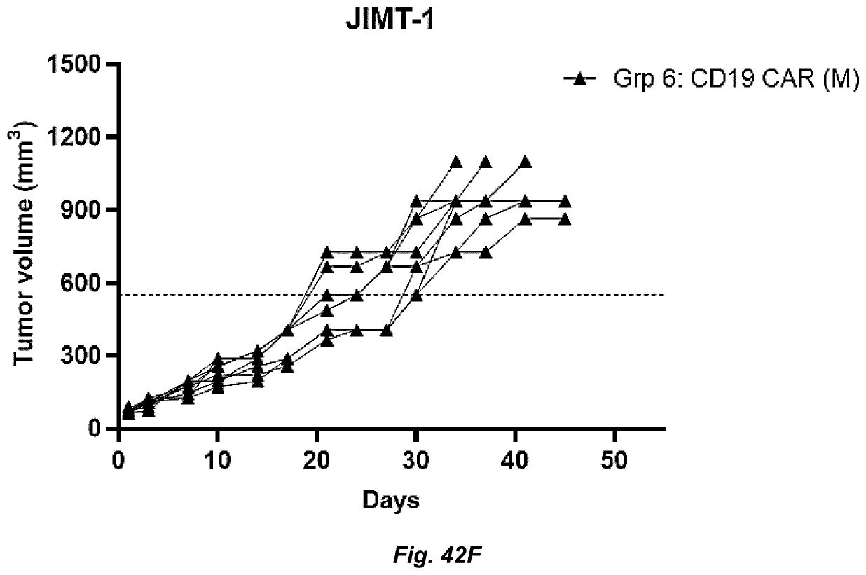

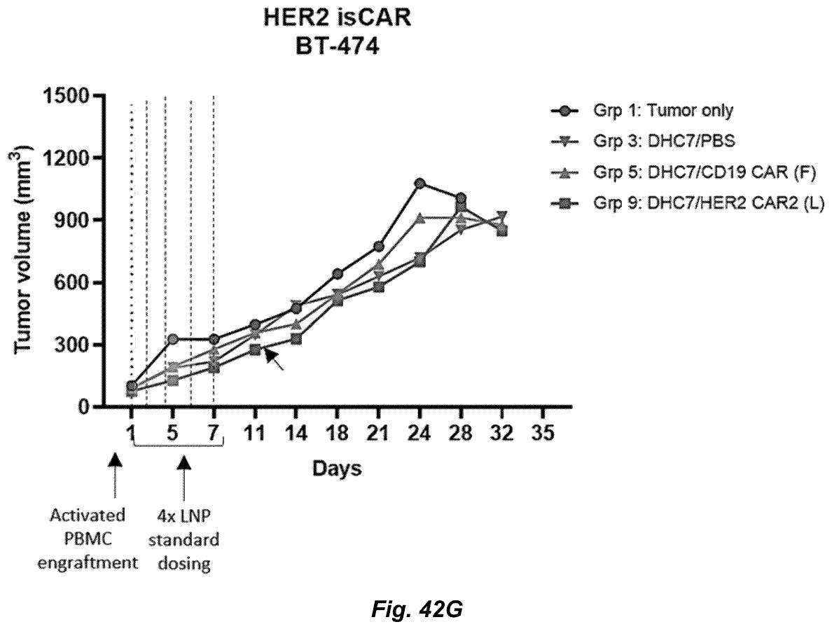

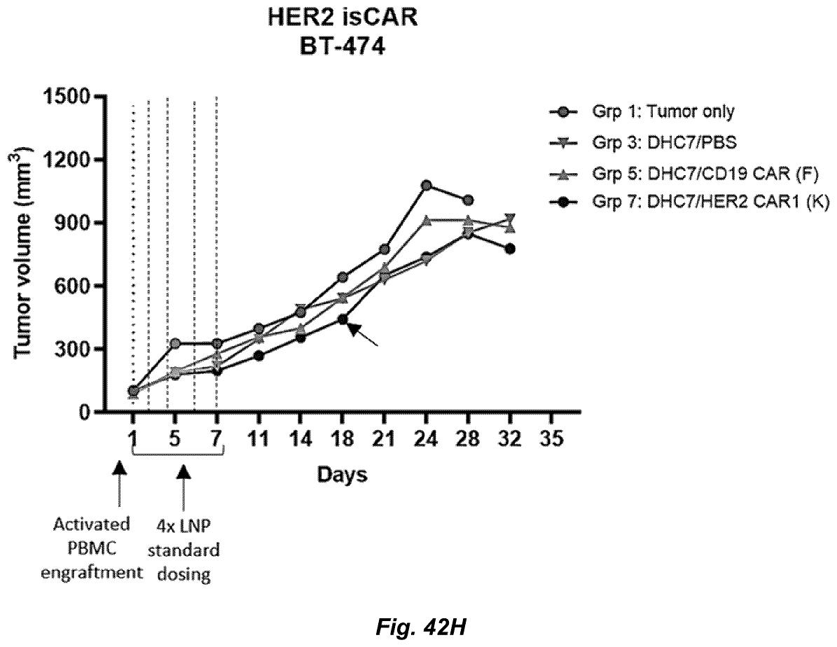

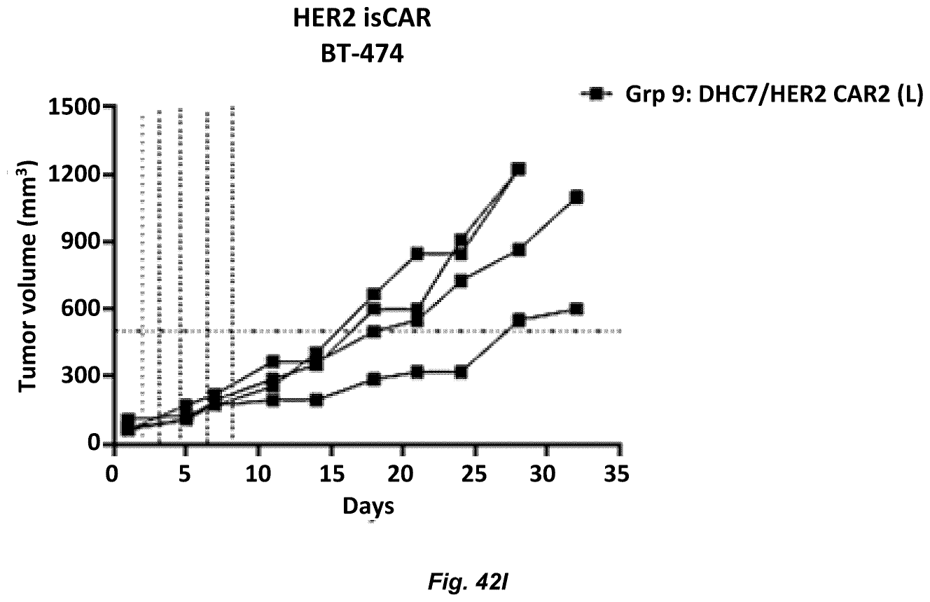

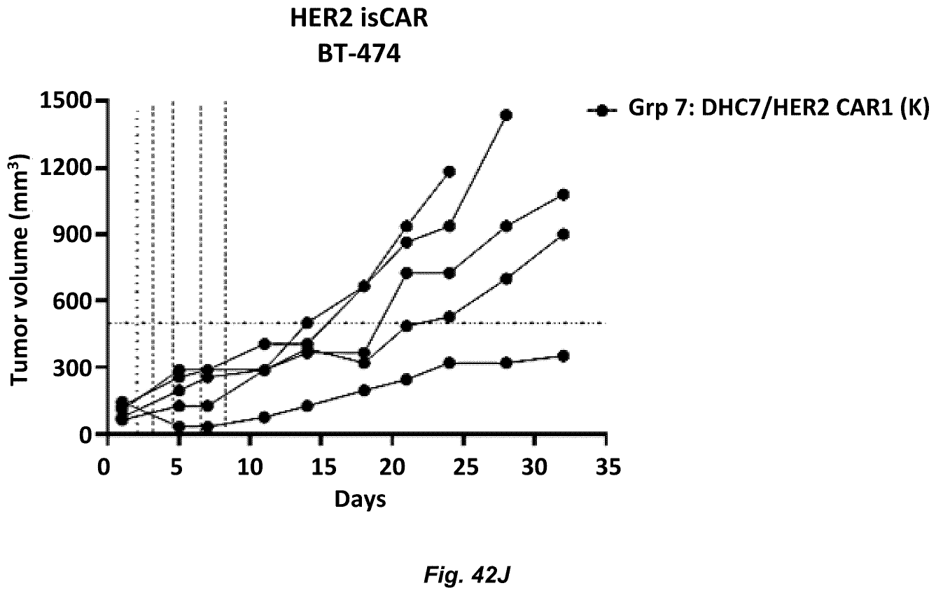

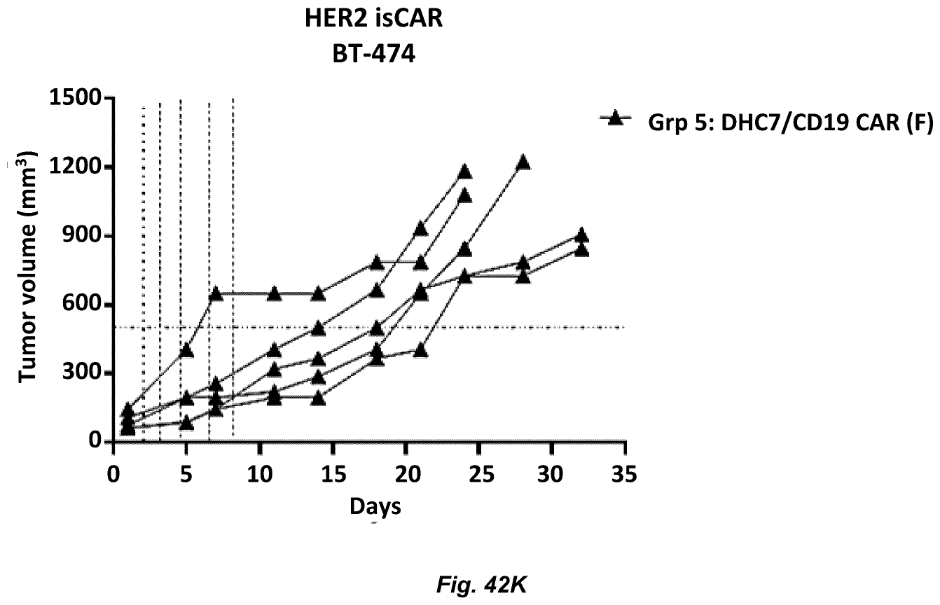

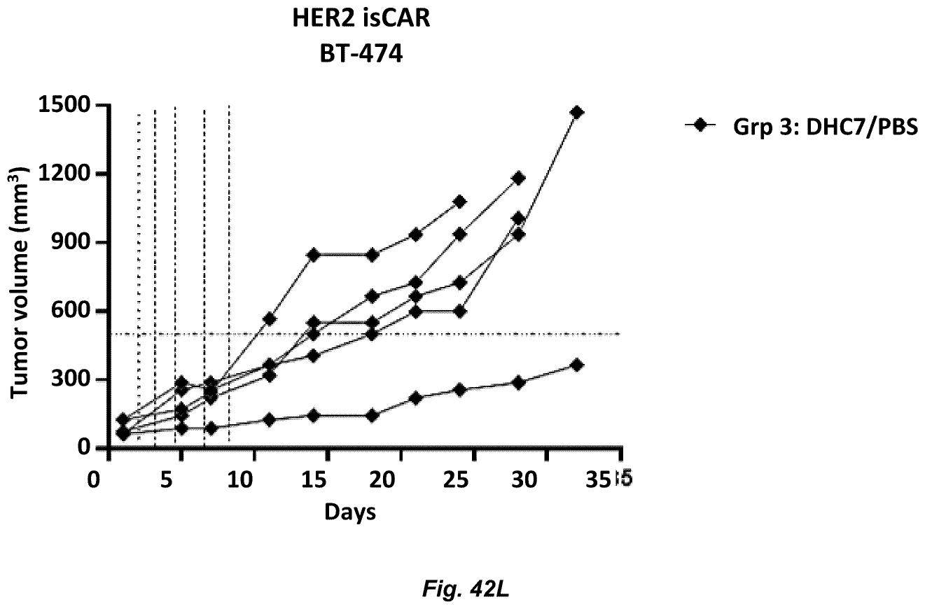

Female NSG immune deficient mice (aged 8 to 12 weeks) with 1×10⁷ JIMT-1 or BT-474 tumor cells (in subcutaneous flank engraftment) were prepared to have an average tumor size of 50-100 mm³. 1×10⁷ human PBMC T cells from two different donors were activated with anti-CD3 and anti-CD8 for 72 hours, then activation was removed and cells were prepared in PBS for injection. On the fifth day after human PBMC thawed, 200 μL of 50×10⁶/mL of the stimulated human PBMC T cells were injected into the tail vein of mice intravenously. A day after the activated human PBMC T cells were injected, the mice were dosed with either docetaxel (control, no PBMC) at 10 mL/kg, PBS (control), or LNP comprising circular RNAs encoding HER2.28° C., HER2.BBC or CD19.28 CAR at 3 mg/kg intravenously. Circular RNAs further comprised internal ribosome entry sites (IRESes) derived from Caprine Kobuvirus or Hunnivirus. LNPs comprised the ionizable referenced in the preceding example. The mice were dosed every other day (within a four-hour timeframe) for a total of 4 dosages. FIGS. 42A-42G provides total tumor volume accumulated over the span of 50 days for mice comprising the JIMT-1 (FIGS. 42A-42F) and BT-474 (FIGS. 42F-42L) tumor cells.

### Example 18: Circular RNA Encoding HER2 CAR

Frozen human T cells were thawed and activated with Stemcell CD3/CD28 antibody cocktail for 72 hours. Post activation T cells were electroporated with circular RNA as described herein comprising different IRES sequences and the HER2 CAR sequences as shown in Construct N and Construct O above, with either 28z or BBz domains. T cells that were electroporated (EP) but had no circular RNA (mock) or had a circular RNA encoding CD19 CAR served as controls. After EP, cells were allowed to recover for 24 hours, following which they were assessed for expression by FACS at 24, 48 and 72 hours post transfection. HER2 CAR was detected using soluble HER2 fluorokines (soluble proteins conjugated to fluorophores).

Incucyte assays were performed. 24 hours after EP, cells expressing the circular RNA encoding HER2 CAR were plated on Incucyte plates at an E: T of 1:1 with HER2 positive, GFP positive BT474 target cells in T cell growth media without IL2. Annexin V, which stains dead cells, was added to the co-culture to track the accumulation of dead cells. The plate was read on the Incucyte instrument for 5 days. Target cell death was assessed by analyzing the percent of green target cells that were stained with Annexin V and comparing to the total GFP positive population in the well as well as to the mock control.

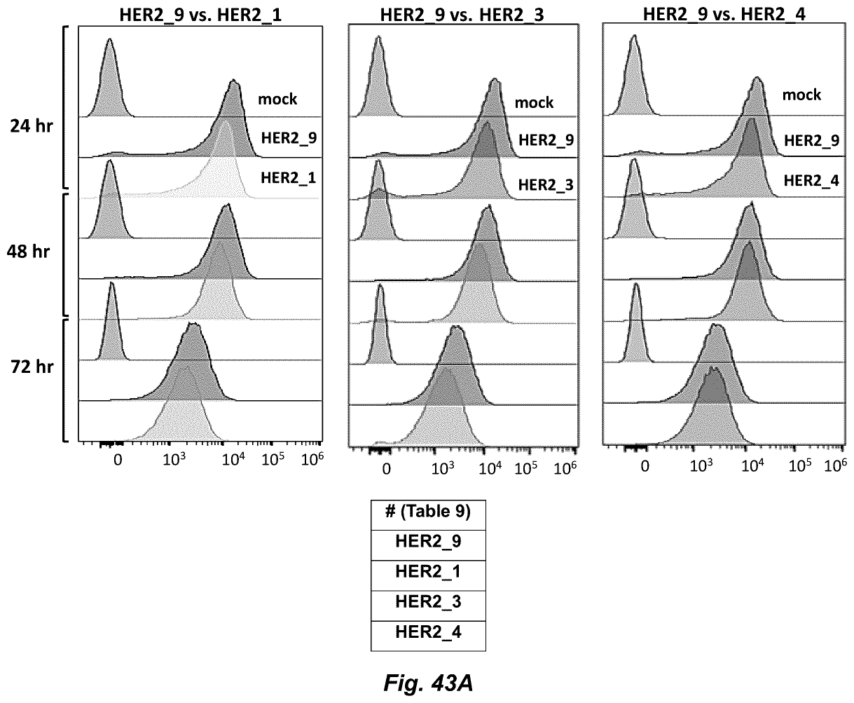

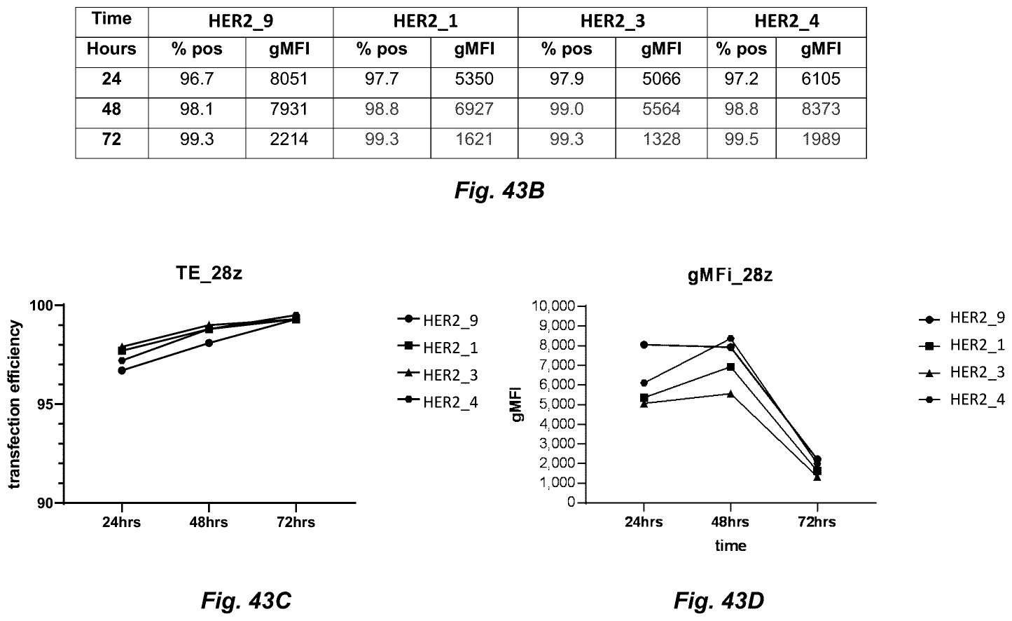

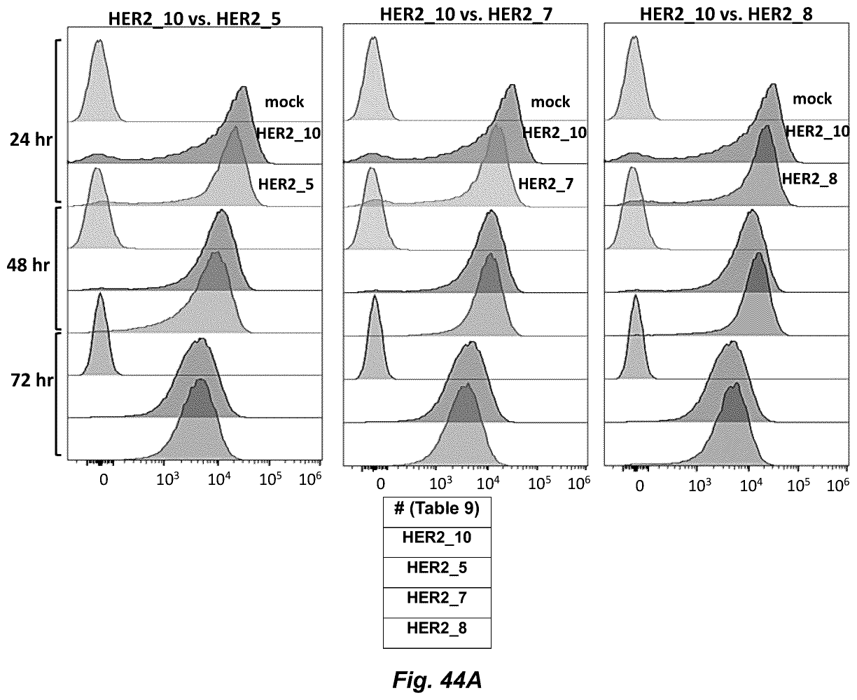

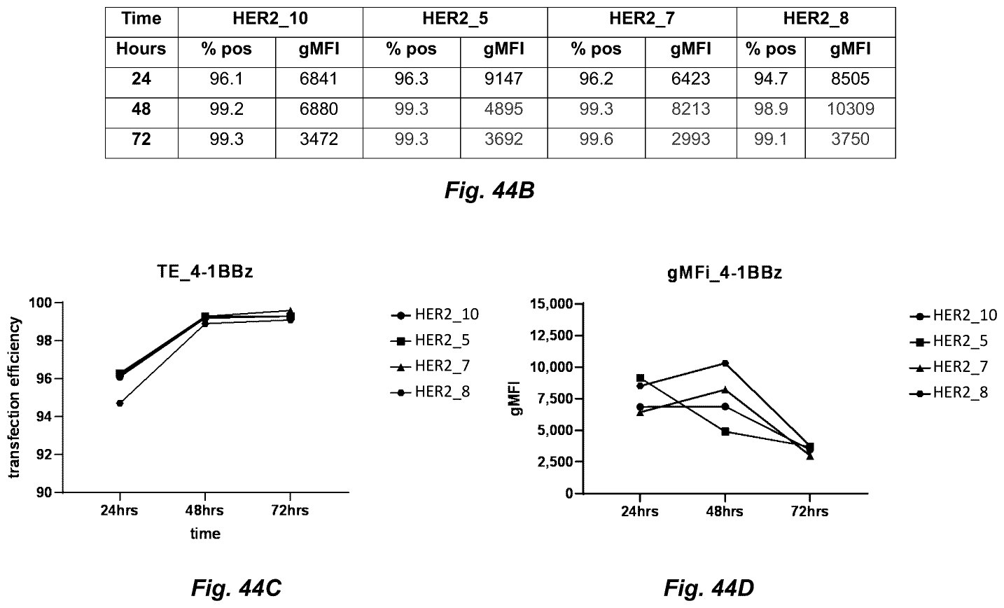

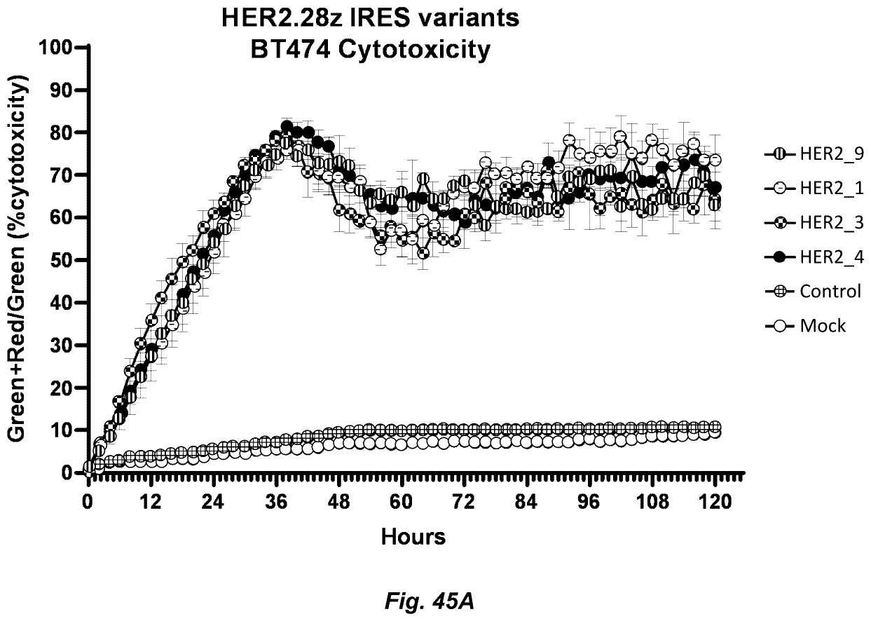

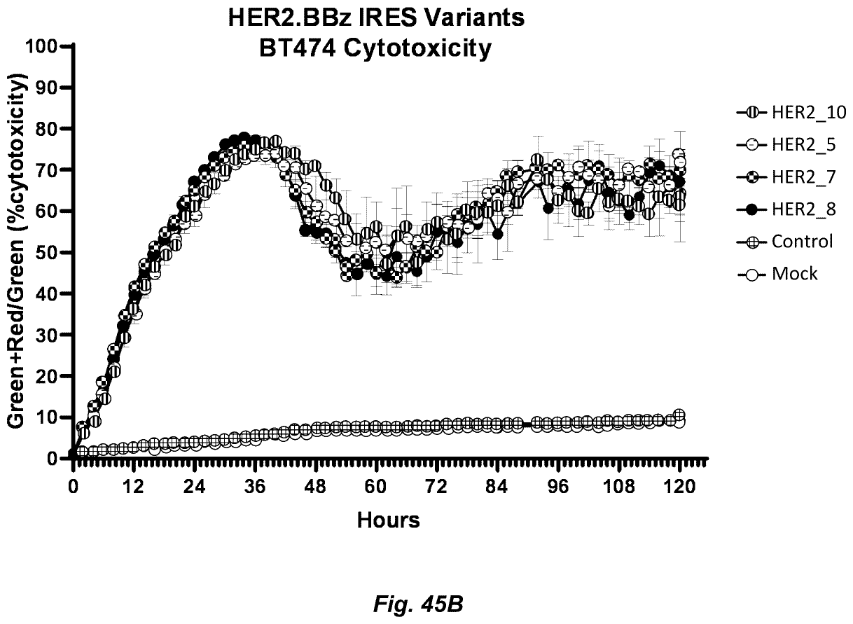

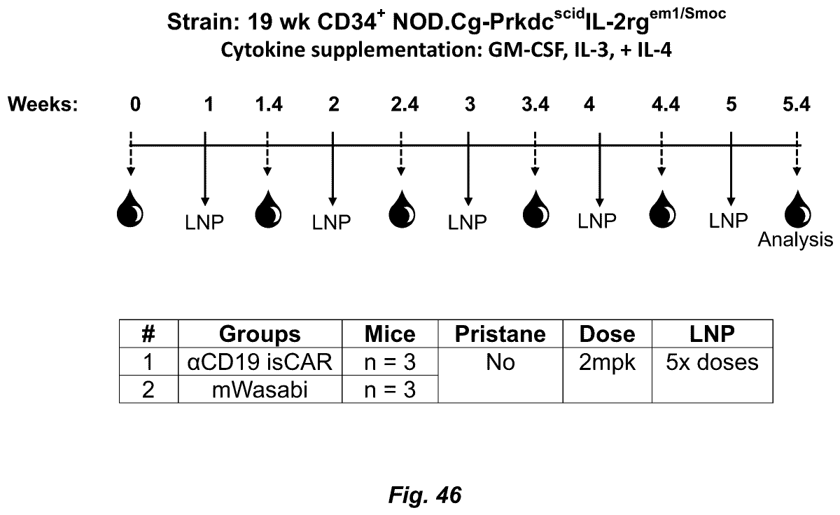

Expression kinetics were assessed for circular RNAs encoding HER2 CAR and comprising different IRES sequences over 72 hours. Cytotoxicity function of BT474 target cells by Incucyte was assessed for circular RNAs encoding HER2 CAR and comprising different IRES sequences. See, e.g., FIGS. 43A-D, FIGS. 44A-D, FIGS. 45A-B, and FIG. 46.

### Example 19: Use of circular RNA encoding CD19 CAR in autoimmune disease

The ability of circular RNA encoding CD19 CAR to deplete human B cells in a CD34+engrafted humanize mouse model was assessed using a CD34+NOD.Cg-PrkdcscidIL-2rgem 1/Smoc strain at 19 weeks. The CD34+Humanized Mice (HiMice) were generated by Invivocue. Five- to six-week-old, female NOD-PrkdescidIL2rgem l/Smoc mice, at approximately 16-22g, were sub-lethally irradiated and engrafted with human CD34+ hematopoietic stem cells via i.v. During the humanization process, mice were monitored over the course of 12 to 14 weeks post engraftment to ensure immune lineage differentiation & maturation. HiMice with minimal 10% of human CD45+reconstitution at week 12- or 14 were used in this experiment. HiMice were also supplemented with cytokines GM-CSF, IL-3, and IL-4 to enhance myeloid lineage, B cell differentiation, and preserve T cell development. Once engraftment was confirmed, a total of 5 doses of either mWasabi oRNA encapsulated in an LNP disclosed herein or CD19 CAR oRNA encapsulated in the same LNP were administered i.v. weekly. Peripheral blood was collected post 3 days of each LNP-ORNA injection and analyzed for immune subsets. Spleens were harvested and processed 3 days after the 5th LNP-ORNA injection for immune profiling.

Cells from blood or spleen were first stained with 50 μl of live/dead solution (1:400 dilution in PBS) for 10 minutes at room temperature. The cells were then pelleted by centrifugation and resuspended with mouse and human Fc receptor blocking reagent (in 25 μl of FACS buffer) for 10 minutes at room temperature to prevent non-specific binding. Subsequently, fluorescent-labeled surface markers (in 25 μl of FACS buffer) were added to the cells. After approximately 30 minutes of incubation in the dark, the cells were washed before acquiring the FACS data using a Fortessa™ X-20 flow cytometer (BD Biosciences) with FACSDiva software. The FACS data were then analyzed with FlowJo software (Tree Star Inc).

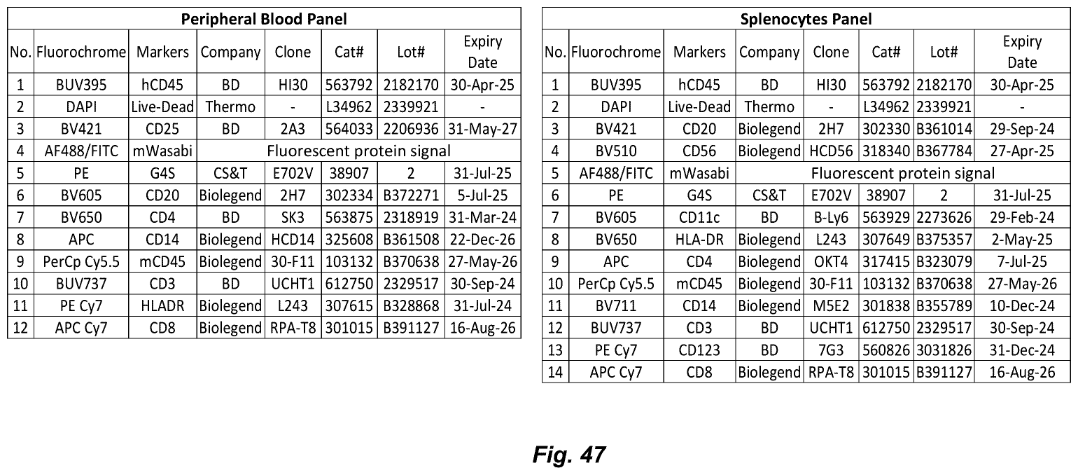

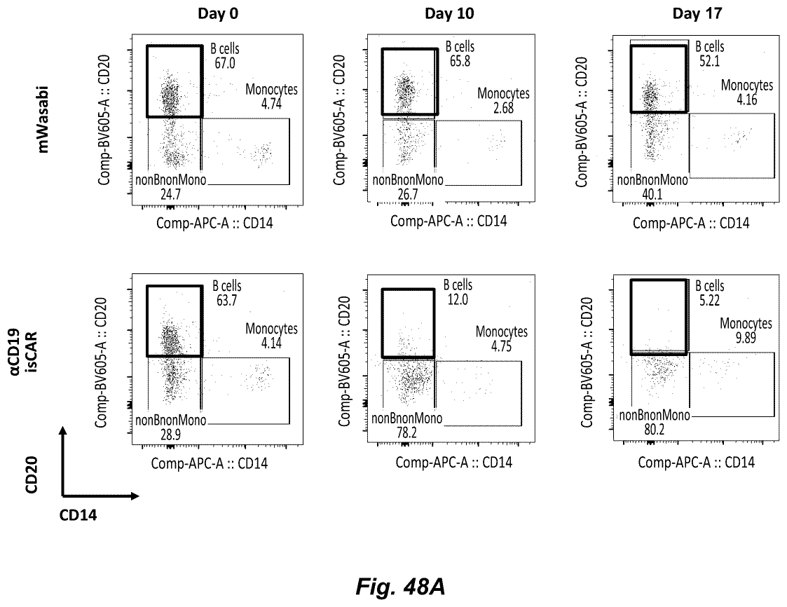

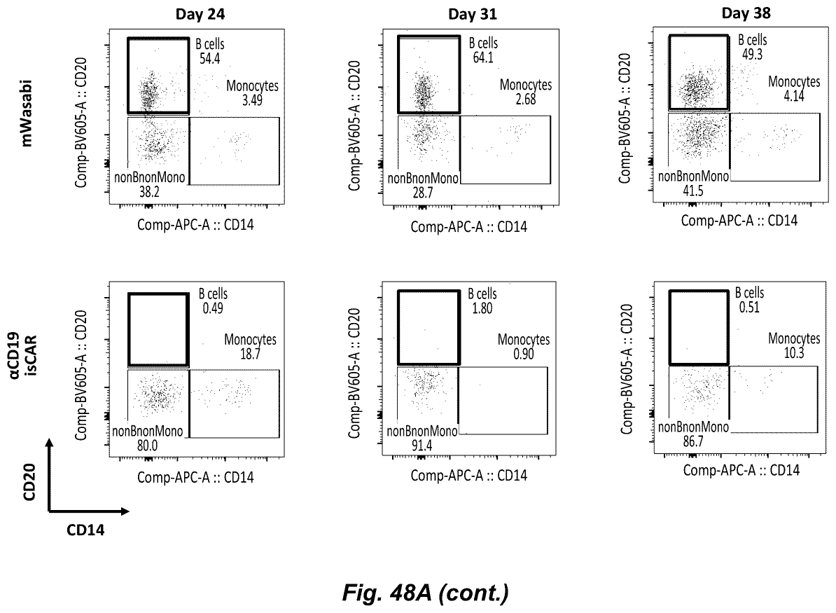

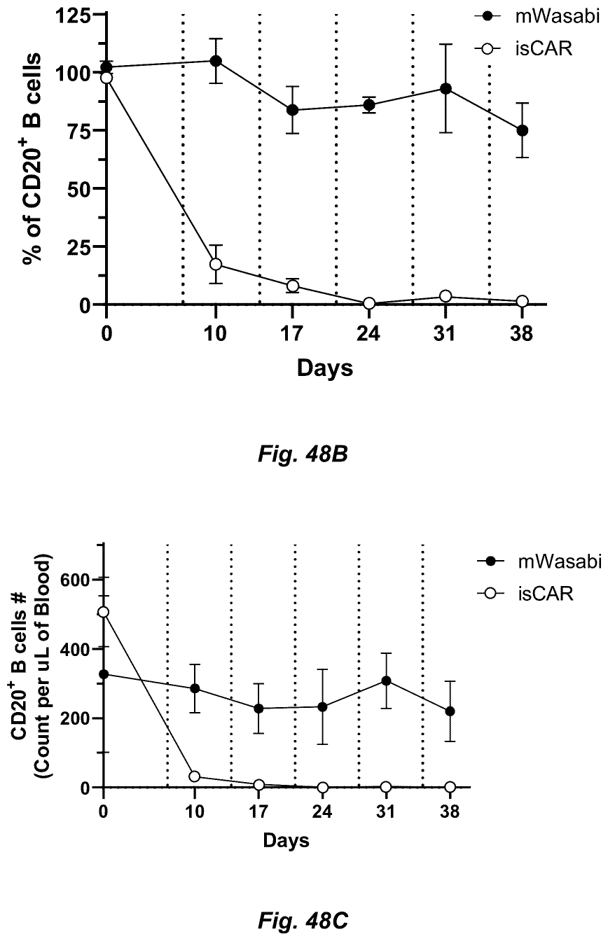

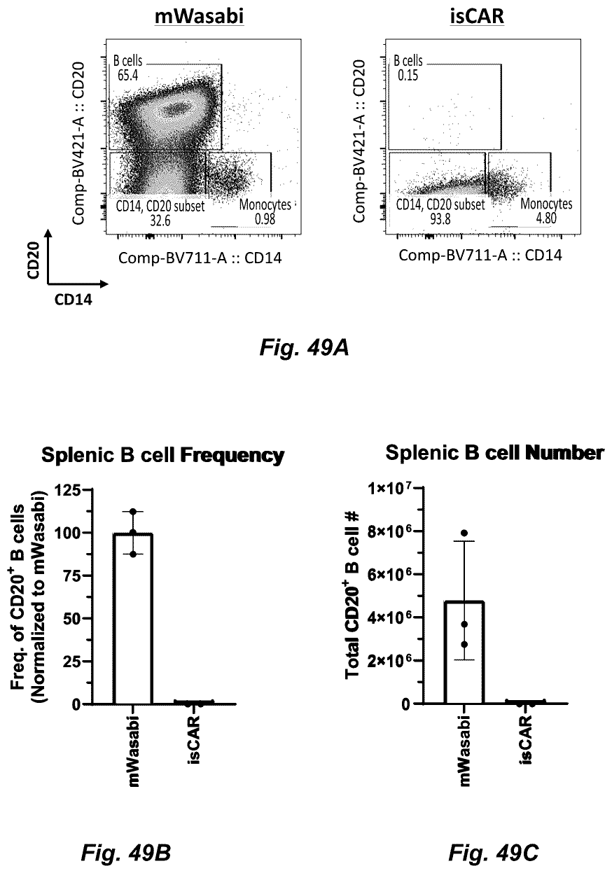

The circular RNA encoding CD19 CAR mediated B-cell specific depletion in CD34+humanized mice. B cell-mediated killing in groups treated with CD19 CAR ORNA encapsulated in LNP was maintained upon 5 doses within the peripheral blood and spleen. Minimal LNP effect was observed on total CD20+B cell frequency and count in the peripheral blood. See, e.g., FIG. 47, FIGS. 48A-C, FIGS. 49A-C.

### Example 20: Circular RNA Encoding CD19 CAR in NK Cells

RAJI control in natural killer (NK) cells using circular RNA encoding CD19 CAR in NOG-IL15 mice was assess. See, e.g., Katano et al., Long-term maintenance of peripheral blood derived human NK cells in a novel human IL-15-transgenic NOG mouse, Sci Rep (2017), the contents of which are hereby incorporated by reference herein (“mouse strain expressing transgenic human interleukin-15 (IL-15) using the severe immunodeficient NOD/Shi-scid-IL-2Rγnull (NOG) mouse genetic background (NOG-IL-15 Tg). Human natural killer (NK) cells, purified from the peripheral blood (hu-PB—NK) of normal healthy donors, proliferated when transferred into NOG-IL-15 Tg mice”).

NOG-IL15 mice were engrafted with a CD19+Raji-luciferase cell line at Day 0. On Day 3, primary human NK cells purified from peripheral blood were engrafted into recipient animals. On Day 8, mice were left untreated, or treated i.v. with vehicle, circular RNA encoding mOX40L CAR encapsulated in LNP (1 mg/kg), or circular RNA encoding CD19 CAR encapsulated in LNP (1 mg/kg). Mice were treated every two days for 10 doses. Tumor burden was assessed using IVIS imaging as described herein.

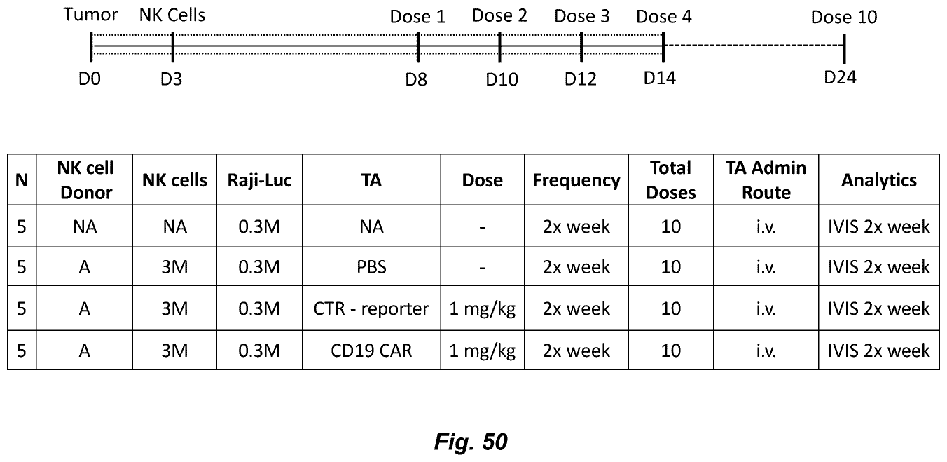

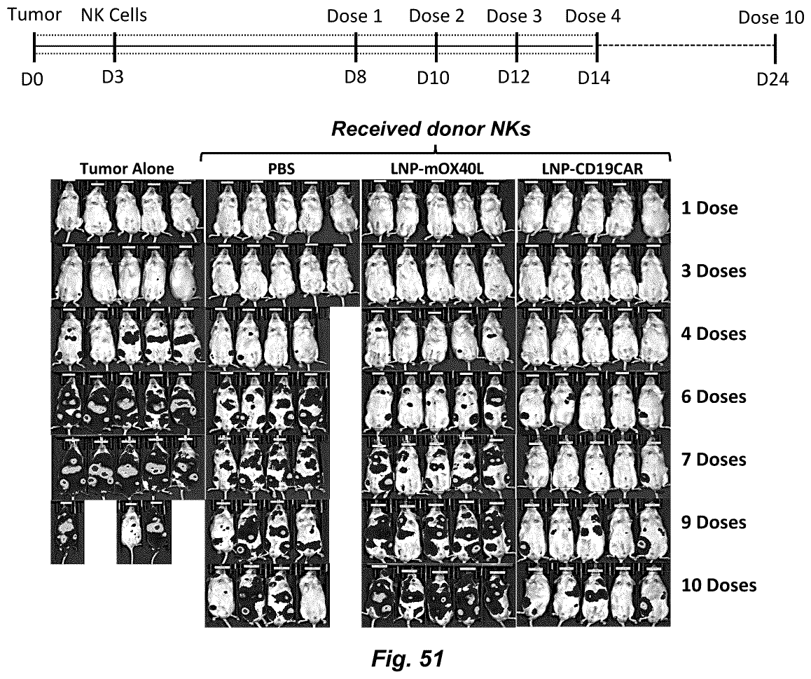

Tumor clearance was observed in this mouse model mouse model. Mice treated with circular RNA encoding CD19 CAR encapsulated in LNP exhibited tumor control until the study endpoint at Day 24. See, e.g., FIG. 50 and FIG. 51.

### Example 21: Circular RNA encoding CD19 CAR in macrophages

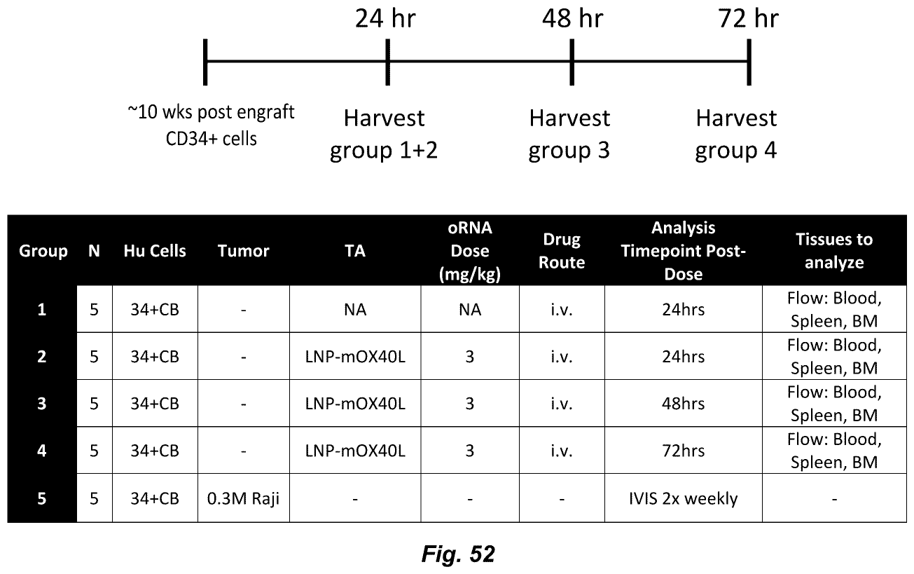

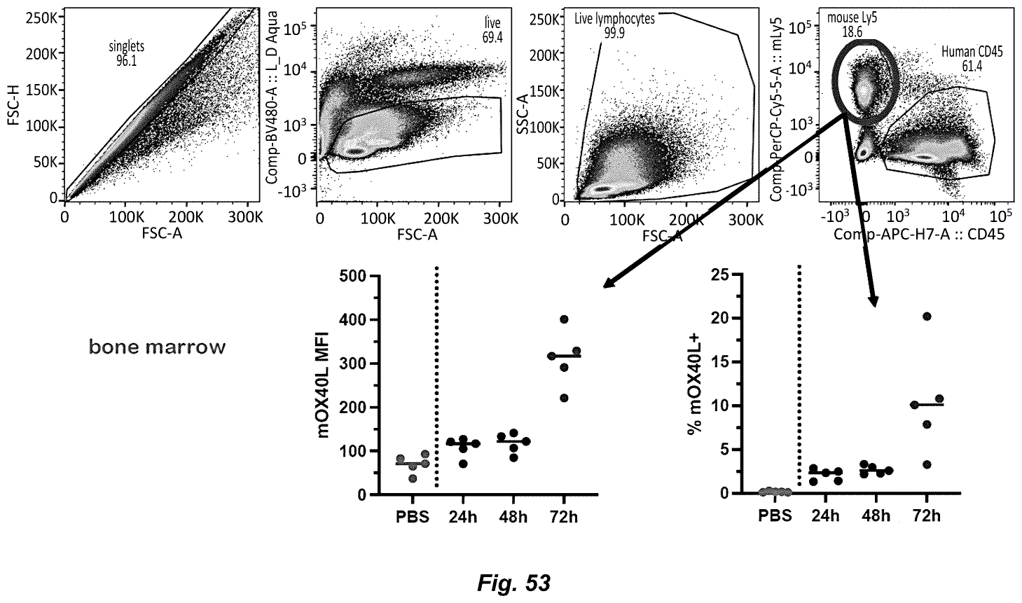

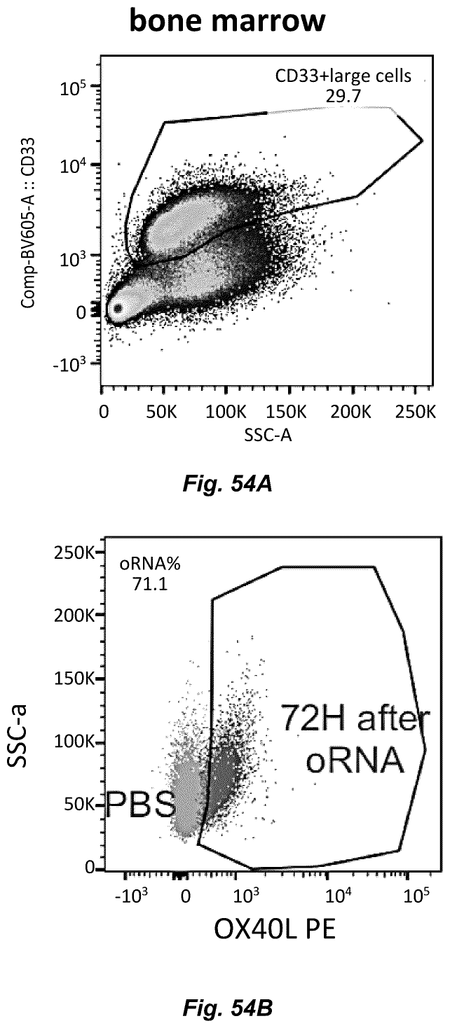

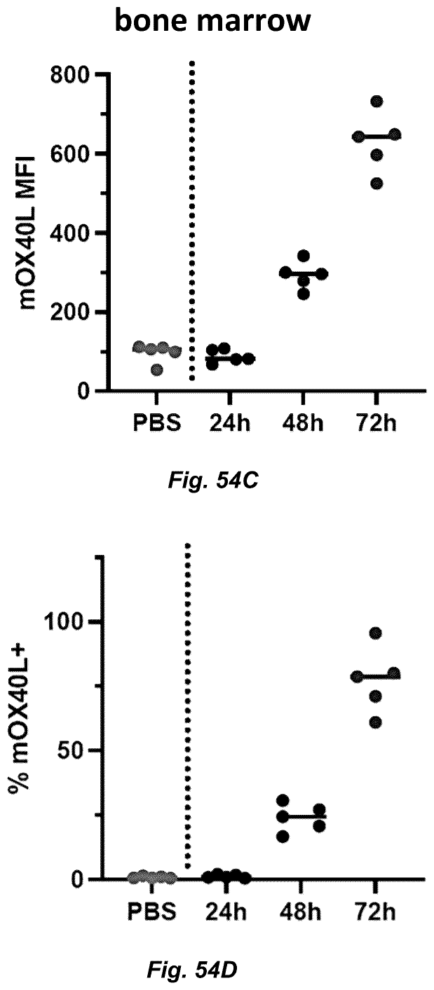

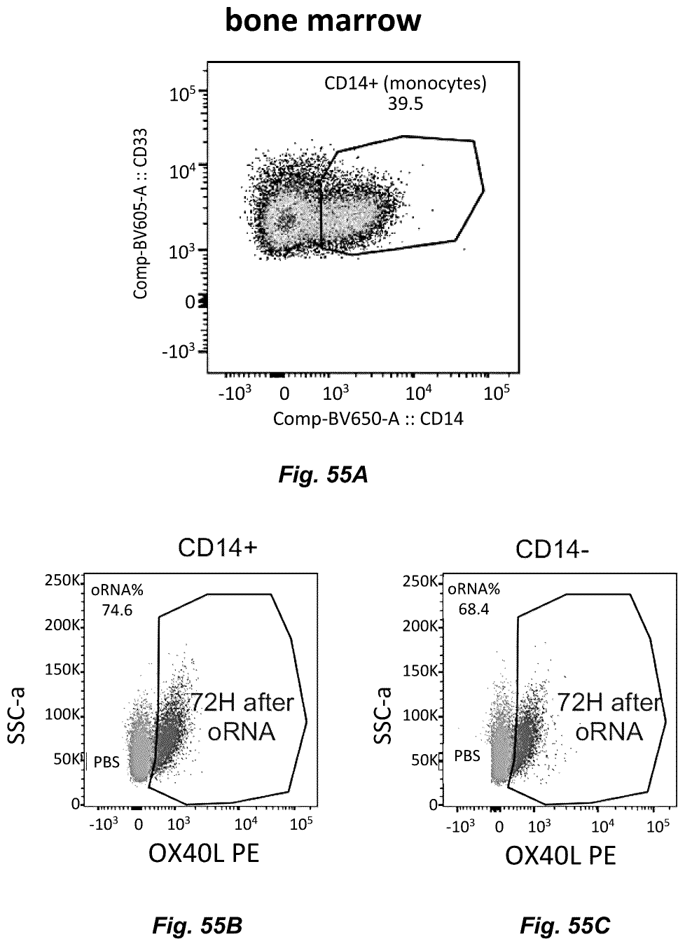

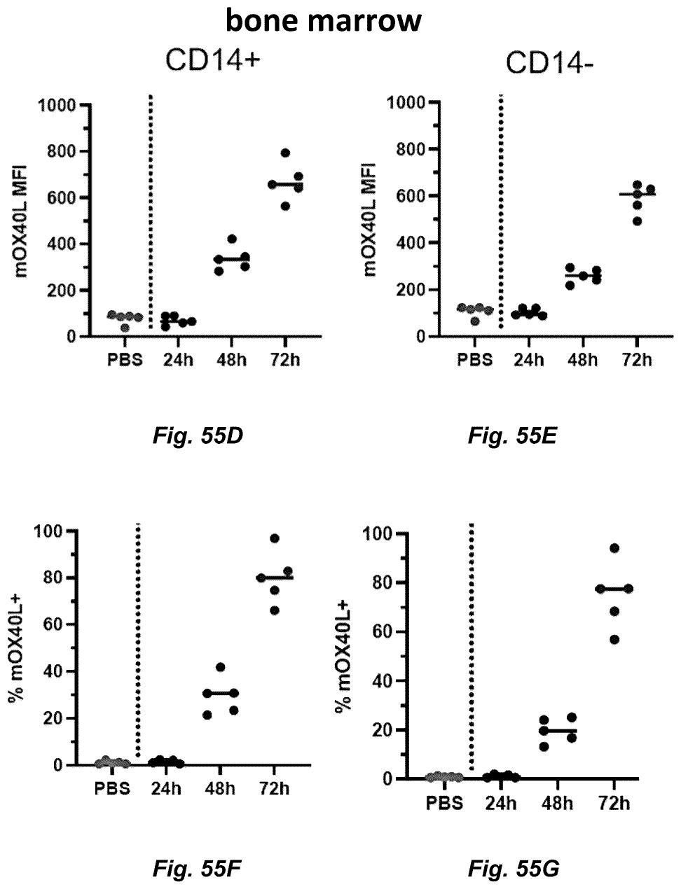

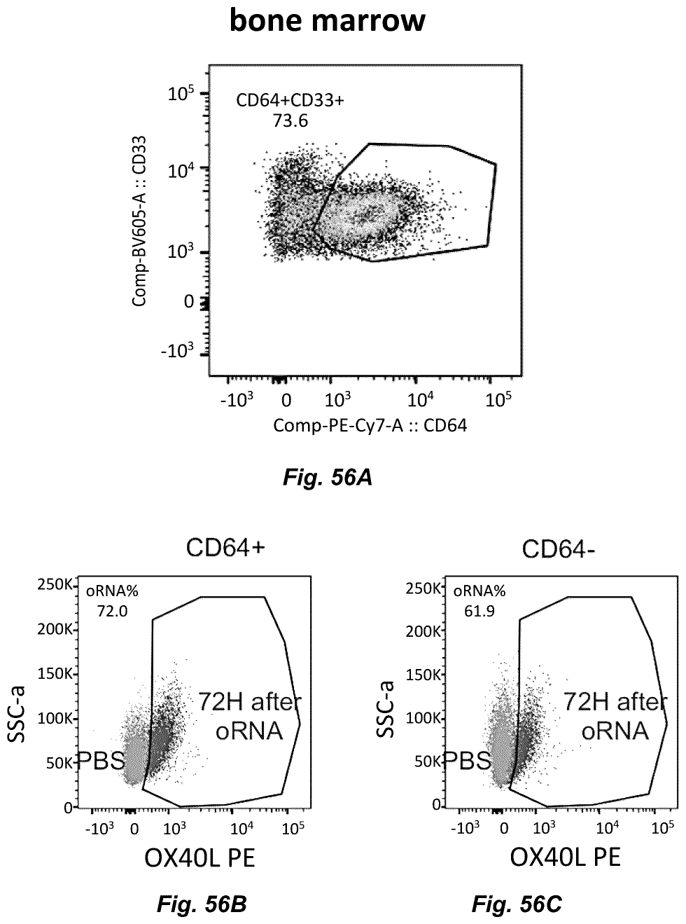

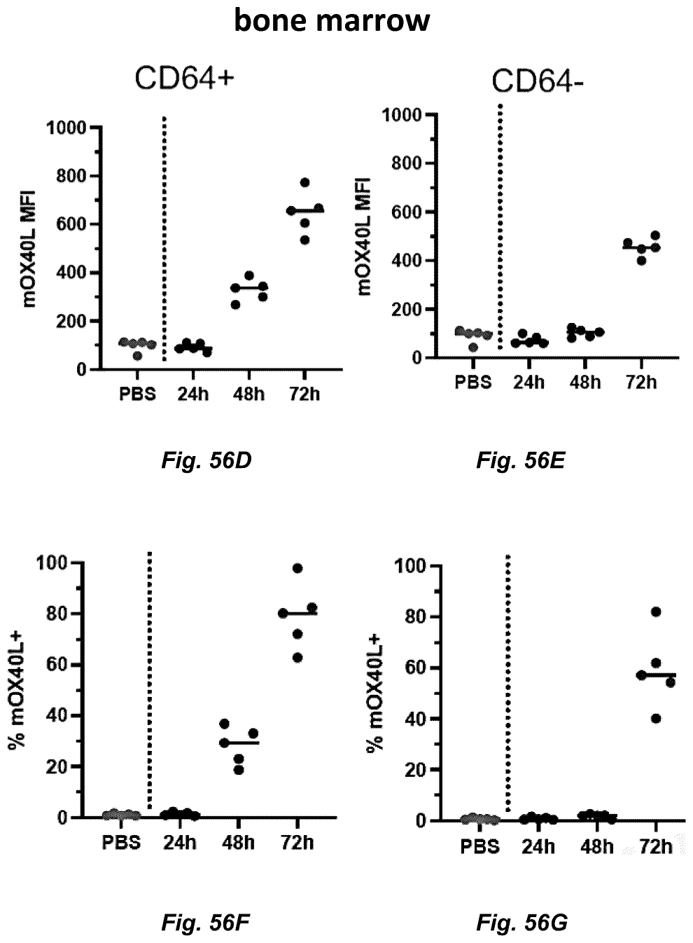

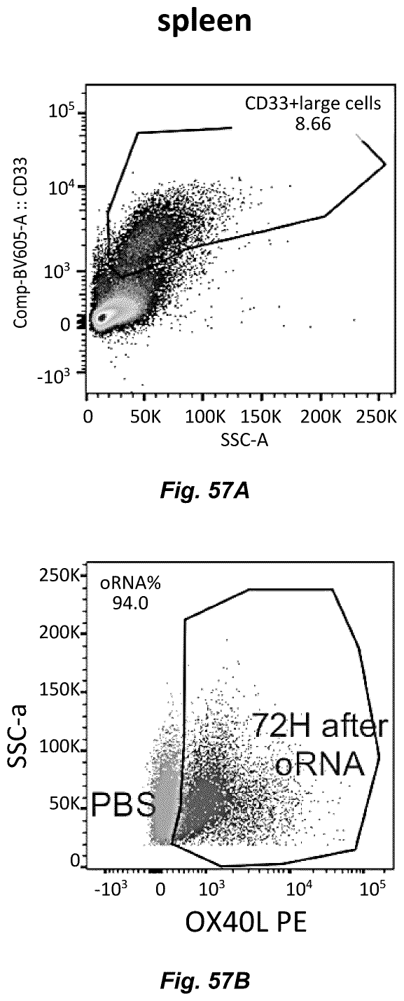

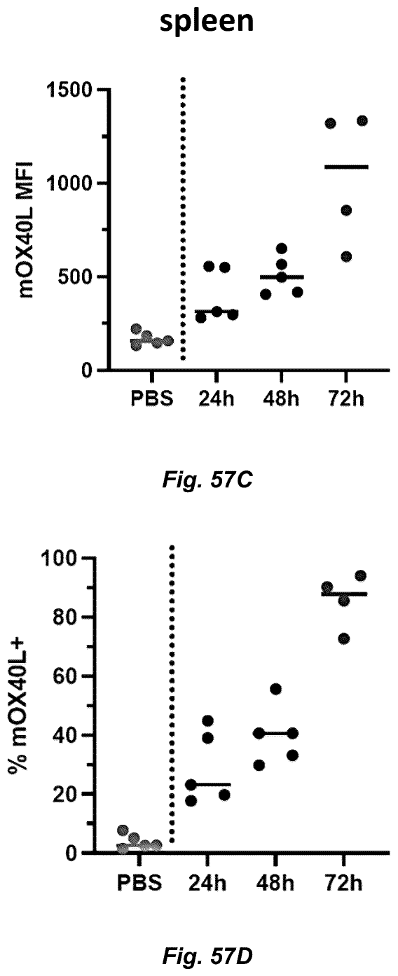

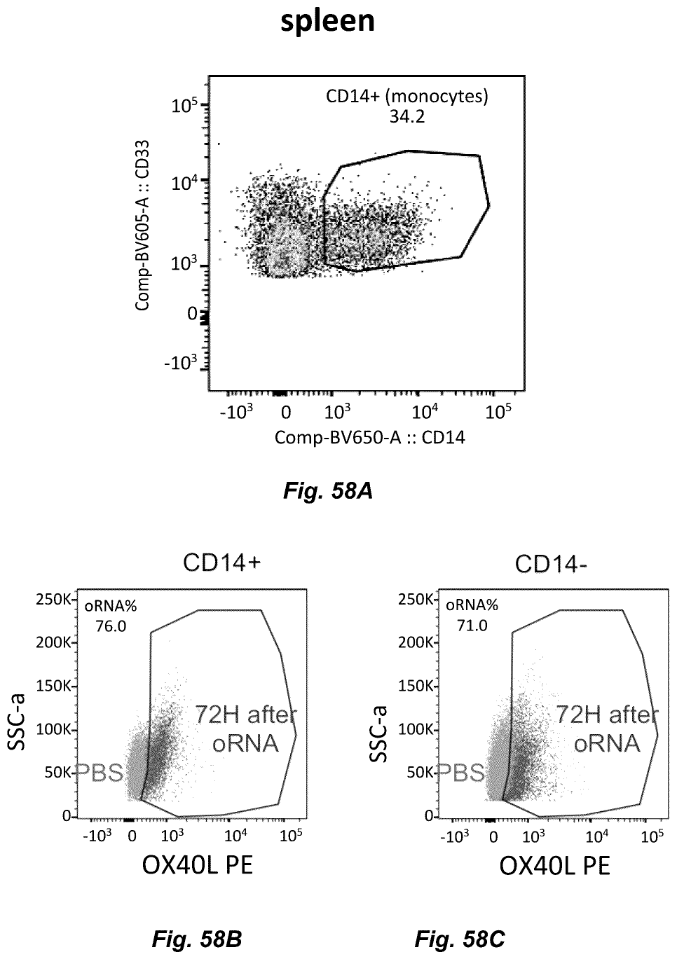

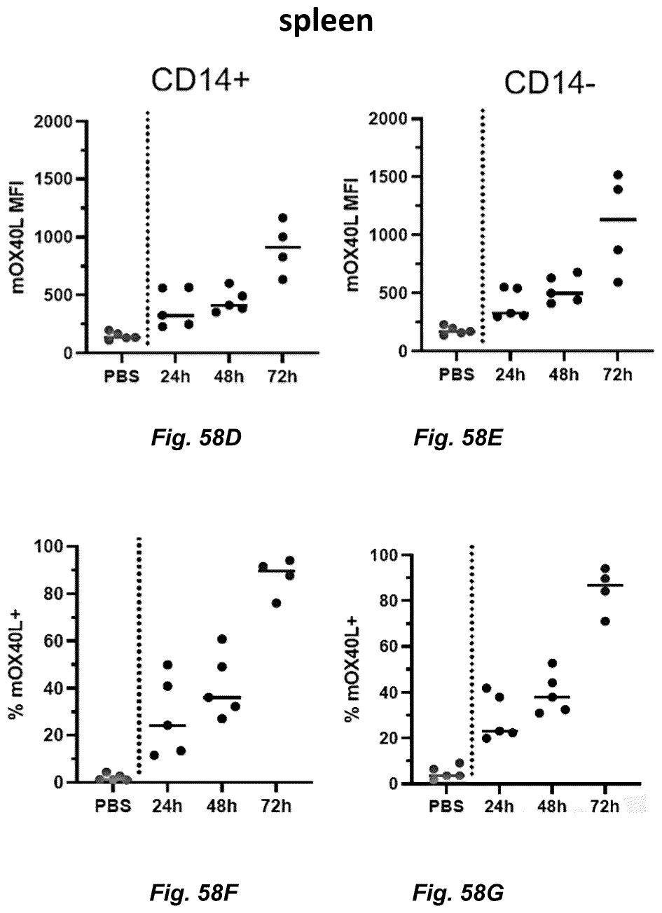

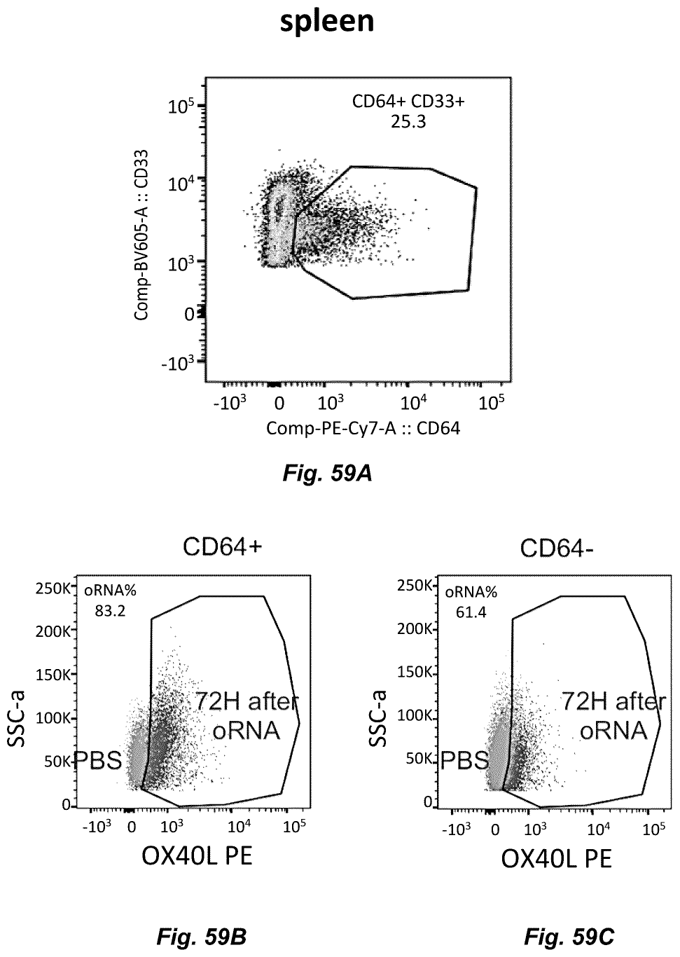

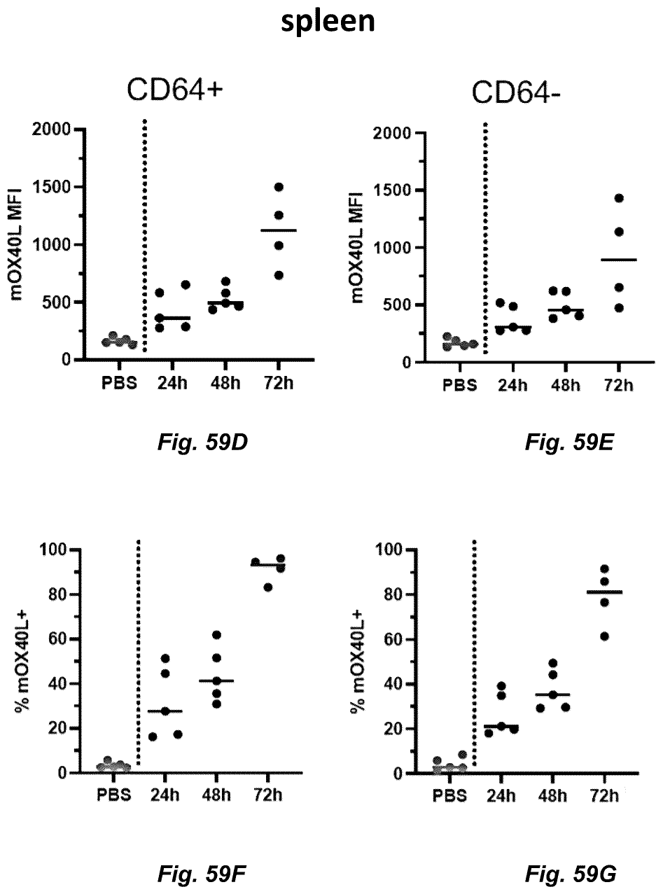

NSG-QUAD mice were engrafted with human CD34+ cord blood. At 10 weeks post-engraftment, animals were left untreated (control) or treated with circular RNA comprising a sequence for mOX40L encapsulated in LNP (see FIG. 52). Animals were sacrificed after 24, 48, or 72 hours. mOX40L expression was analyzed by flow cytometry on CD33+, CD33+CD14+ or CD33+CD64+myeloid cells in the blood, bone marrow and spleen. mOX40L was detected on the surface of CD33+myeloid cells in the bone marrow and spleen at each timepoint, with peak expression observed at 72 hours LNP-circRNA treatment. See, e.g., FIG. 53, FIGS. 54A-D, FIGS. 55A-G, FIGS. 56A-G, FIGS. 57A-D, FIGS. 58A-G, and FIGS. 59A-G.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

1-108. (canceled)

109. A circular RNA construct comprising:
(A) a translation initiation element comprising a sequence that is at least 80% identical to any one of SEQ ID NO: 8, SEQ ID NOs: 1-7, SEQ ID NOs: 9-18, or fragment thereof, and
(B) at least one expression sequence that encodes:
a. a heavy variable region that is at least 95% identical to a heavy variable region of SEQ ID NO: 29 and a light variable region that is at least 95% identical to a light variable region of SEQ ID NO: 29, or binding fragments thereof;
b. a heavy variable region that is at least 95% identical to a heavy variable region of SEQ ID NO: 122 and a heavy variable region that is at least 95% identical to a heavy variable region of SEQ ID NO: 122, or binding fragments thereof;
c. a heavy variable region that is at least 95% identical to a heavy variable region of SEQ ID NO: 121 and a light variable region that is at least 95% identical to a light variable region of SEQ ID NO: 121, or binding fragments thereof; and/or
d. a heavy variable region that is at least 95% identical to a heavy variable region of SEQ ID NO: 117 and a light variable region that is at least 95% identical to a light variable region of SEQ ID NO: 117, or binding fragments thereof.

110. The circular RNA construct of claim 109, wherein the translation initiation element comprises a sequence that is at least 90%, at least 95%, or at least 100% identical to SEQ ID NO: 8 or fragment thereof.

111. The circular RNA construct of claim 109, wherein the circular RNA construct comprises an expression sequence that encodes a heavy variable region that is at least 95% identical to a heavy variable region of SEQ ID NO: 29 and a light variable region that is at least 95% identical to a light variable region of SEQ ID NO: 29, or binding fragments thereof.

112. The circular RNA construct of claim 109, wherein the binding fragments thereof are complementarity determining regions (CDRs).

113. The circular RNA construct of claim 109, wherein the expression sequence encodes a single chain antibody fragment (scFv).

114. The circular RNA construct of claim 113, wherein the variable regions are linked by a linker.

115. The circular RNA construct of claim 109, wherein the circular RNA further comprises a polyA region, at least one miRNA binding site, and/or at least one miR-122 binding site.

116. The circular RNA construct of claim 109, wherein the expression sequence is codon optimized.

117. The circular RNA construct of claim 109, wherein the expression sequence further encodes at least one of a signal peptide, a hinge domain, a transmembrane domain, a costimulatory domain, and a signaling domain.

118. The circular RNA construct of claim 117, wherein:
(a) the hinge domain is derived from a ErbB2, glycophorin A (GpA), CD2, CD3 delta, CD3 epsilon, CD3 gamma, CD4, CD7, CD8a, CD8 [T CD1 1a (IT GAL), CD1 1b (IT GAM), CD1 1c (ITGAX), CD1 1d (IT GAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (0X40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRT AM), CD357 (TNFRSF18), inducible T cell co-stimulator (ICOS), LFA-1 (CD1 1a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), or PAG1/CBP;
(b) the transmembrane domain is derived from ErbB2, glycophorin A (GpA), 4-1BB/CD137, activating NK cell receptors, an immunoglobulin protein, B7-H3, BAFFR, BFAME (SEAMF8), BTEA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD1 1a, CD1 1b, CD1 1c, CD1 1d, CDS, CEACAM1, CRT AM, cytokine receptor, DAP-10, DNAMI (CD226), Fc gamma receptor, GADS, GITR, HVEM (EIGHTR), IA4, ICAM-1, ICAM-1, Ig alpha (CD79a), IE-2R beta, IE-2R gamma, IE-7R alpha, inducible T cell costimulator (ICOS), integrins, ITGA4, ITGA4, ITGA6, IT GAD, ITGAE, ITGAE, IT GAM, ITGAX, ITGB2, ITGB7, ITGB1, KIRDS2, EAT, LFA-1, LFA-1, a ligand that specifically binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6;
(c) the costimulatory domain is selected from a CD137 costimulatory domain or CD28 costimulatory domain; and
(d) the signaling domain is derived from B7-H3, BAFFR, BLAME (SLAMF8), BTLA, CD100 (SEMA4D), CD103, CD160 (BY55), CD18, CD19, CD 19a, CD2, CD247, CD27, CD276 (B7-H3), CD28, CD29, CD3 delta, CD3 epsilon, CD3 gamma, CD3 zeta, CD30, CD4, CD40, CD49a, CD49D, CD49f, CD69, CD7, CD84, CD8alpha, CD8beta, CD96 (Tactile), CD1 1a, CD1 1b, CD1 1c, CD1 1d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, LFA-1, ligand that specifically binds with CD83, LIGHT, LTBR, Ly9 (CD229), Ly108, lymphocyte function-associated antigen-1 (LFA-1; CD1-la/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A), SLAMF7, SLP-76, TNF receptor proteins, TNFR2, TNFSF14, a Toll ligand receptor, TRANCE/RANKL, VLA1, or VLA-6.

119. The circular RNA construct of any one of claim 109, wherein the expression sequence encodes a chimeric antigen receptor.

120. A pharmaceutical composition comprising the circular RNA construct of claim 109; and a transfer vehicle.

121. The pharmaceutical composition of claim 120, wherein the transfer vehicle comprises: (i) an ionizable lipid of Formula (I) 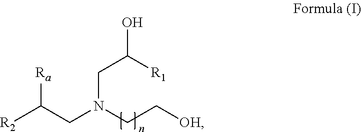
wherein n is an integer between 1 and 4;
Rₐ is hydrogen or hydroxyl; and
R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; or
(ii) an ionizable lipid of Formula (II) 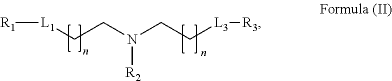
wherein each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

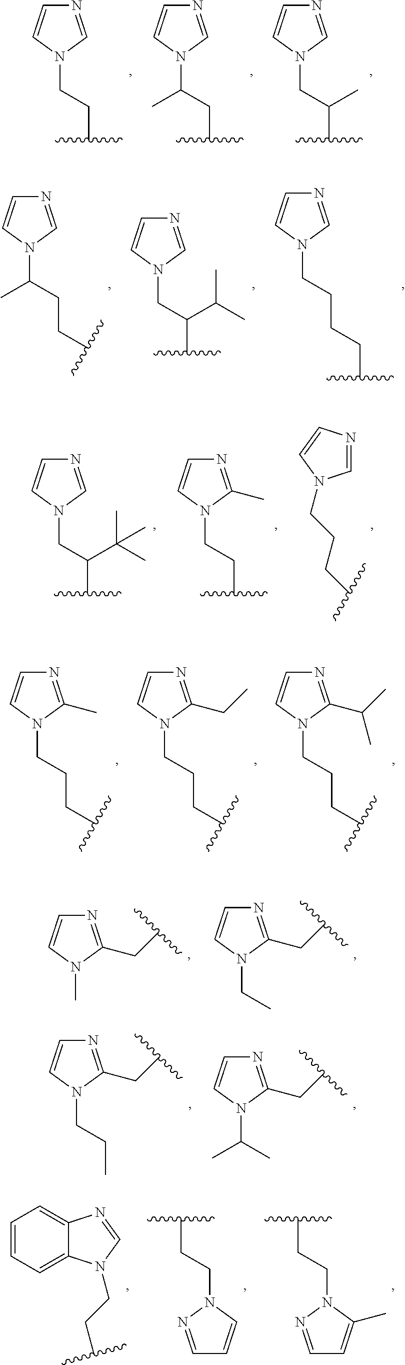

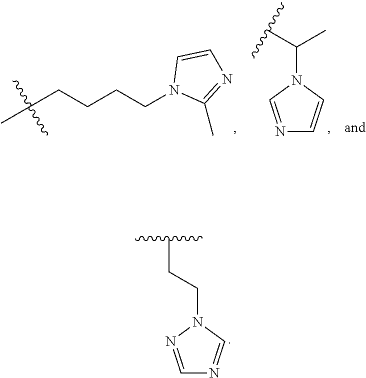

122. The pharmaceutical composition of claim 120, wherein the transfer vehicle has a lipid molar ratio formulation as described in Table 4b.

123. The pharmaceutical composition of claim 120, wherein the transfer vehicle comprises an ionizable lipid selected from:

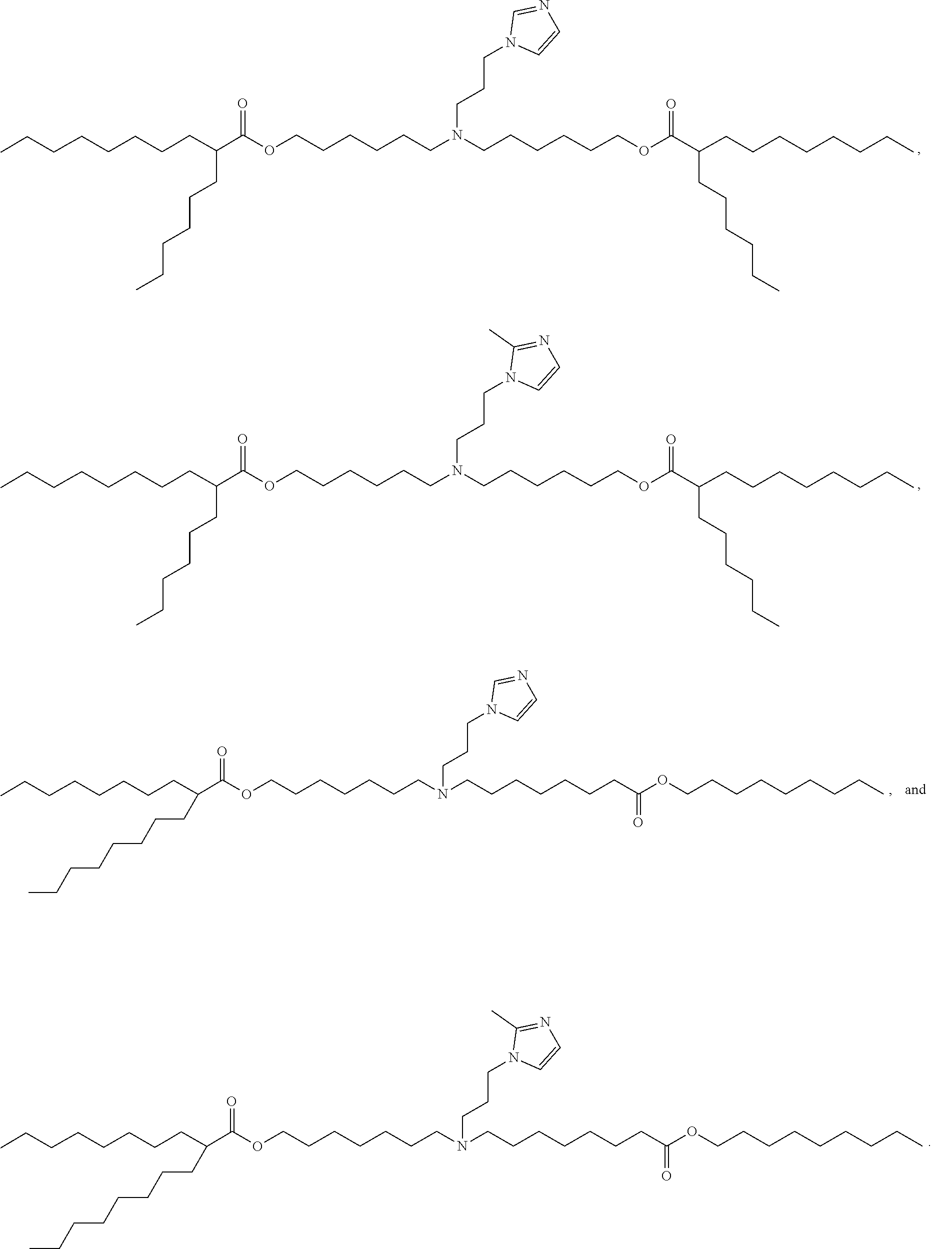

124. The pharmaceutical composition of claim 120, wherein the transfer vehicle further comprises (a) a helper lipid, a structural lipid, and/or a PEG-lipid; and (b) a pharmaceutical salt, buffer, or diluent, or combination thereof.

125. The pharmaceutical composition of claim 124, wherein the transfer vehicle comprises PEG-DSPC.

126. The pharmaceutical composition of claim 120, wherein the transfer vehicle is a lipid nanoparticle.

127. The pharmaceutical composition of claim 120, wherein the transfer vehicle further comprises a targeting moiety selected from a small molecule, scFv, nanobody, peptide, cyclic peptide, di or tri cyclic peptide, minibody, polynucleotide aptamer, engineered scaffold protein, heavy chain variable region, light chain variable region, or a fragment thereof.

128. A method of treating cancer or an autoimmune disorder in a subject by administering an effective amount of a composition comprising the circular RNA construct of claim 109 or a pharmaceutical composition thereof, thereby treating the cancer or autoimmune disorder.

129. A linear precursor RNA polynucleotide comprising:
(A) a translation initiation element comprising a sequence that is at least 80% identical to any one of SEQ ID NO: 8, SEQ ID NOs: 1-7, SEQ ID NOs: 9-18, or fragment thereof, and
(B) at least one expression sequence that encodes:
a. a heavy variable region that is at least 95% identical to a heavy variable region of SEQ ID NO: 29 and a light variable region that is at least 95% identical to a light variable region of SEQ ID NO: 29, or binding fragments thereof;
b. a heavy variable region that is at least 95% identical to a heavy variable region of SEQ ID NO: 122 and a heavy variable region that is at least 95% identical to a heavy variable region of SEQ ID NO: 122, or binding fragments thereof;
c. a heavy variable region that is at least 95% identical to a heavy variable region of SEQ ID NO: 121 and a light variable region that is at least 95% identical to a light variable region of SEQ ID NO: 121, or binding fragments thereof; and/or
d. a heavy variable region that is at least 95% identical to a heavy variable region of SEQ ID NO: 117 and a light variable region that is at least 95% identical to a light variable region of SEQ ID NO: 117, or binding fragments thereof.

130. A DNA vector encoding the RNA polynucleotide of claim 129.

131. A method of preparing a circular RNA construct, the method comprising incubating the linear RNA polynucleotide of claim 129 under suitable conditions for circularization.
